SEQUENCE LISTING (12 sequences)

SEQ ID NO: 1 - DNA, 22 bp, Artificial Sequence
  Description of Artificial Sequence Synthetic primer
caccatggtccgcttcggggac

SEQ ID NO: 2 - DNA, 19 bp, Artificial Sequence
  Description of Artificial Sequence Synthetic primer
ccgttcagtggcctcctcc

SEQ ID NO: 3 - DNA, 23 bp, Artificial Sequence
  Description of Artificial Sequence Synthetic primer
ctagcaccagtgatcctggtctg

SEQ ID NO: 4 - DNA, 20 bp, Artificial Sequence
  Description of Artificial Sequence Synthetic primer
agtgcgttgtgagcgcagta

SEQ ID NO: 5 - DNA, 21 bp, Artificial Sequence
  Description of Artificial Sequence Synthetic primer
caccatggtccagaagagcgg

SEQ ID NO: 6 - DNA, 21 bp, Artificial Sequence
  Description of Artificial Sequence Synthetic primer
tctcagcggatgtagacgcct

SEQ ID NO: 7 - DNA, 22 bp, Artificial Sequence
  Description of Artificial Sequence Synthetic primer
caccatgtatgacgactcctac

SEQ ID NO: 8 - DNA, 23 bp, Artificial Sequence
  Description of Artificial Sequence Synthetic primer
ggtggtcagtagctgtccttagg

SEQ ID NO: 9 - DNA, 21 bp, Artificial Sequence
  Description of Artificial Sequence Synthetic primer
caccatggctgctggctgcct

SEQ ID NO: 10 - DNA, 25 bp, Artificial Sequence
  Description of Artificial Sequence Synthetic primer
agagggtcaccatagatagtgtctg

SEQ ID NO: 11 - DNA, 22 bp, Artificial Sequence
  Description of Artificial Sequence Synthetic primer
caccatgattcgggccttcgct

SEQ ID NO: 12 - DNA, 25 bp, Artificial Sequence
  Description of Artificial Sequence Synthetic primer
agcctgcggactacaggttgctgac

(19) United States
(12) Patent Application Publication
(10) Pub. No.: US 2011/0190300 A1
(43) Pub. Date: Aug. 4, 2011

(54) AMIDE COMPOUNDS AND THE USE THEREOF

(76) Inventors: Akira Matsumura, Osaka (JP); Hidenori Mikamiyama, Osaka (JP); Jiangchao Yao, Princeton, NJ (US)

(21) Appl. No.: 12/602,392

(22) PCT Filed: May 30, 2008
(86) PCT No.: PCT/US08/06855

§ 371 Date: Jun. 25, 2010

Related U.S. Application Data
(60) Provisional application No. 60/924,798, filed on May 31, 2007.

(51) Int. Cl. A61K 31/5377; C07D 211/96; C07D 417/12; C07D 401/12; C07D 413/12; A61K 31/4468; A61K 31/454; A61K 31/4545; A61K 31/4709; G01N 33/53; A61P 9/00; A61P 25/00; A61P 25/08; A61P 29/00; A61P 25/06; A61P 25/18; A61P 25/24; A61P 25/22; A61P 9/12; A61P 9/06

(52) U.S. Cl. 514/237.2; 546/223; 546/209; 546/194; 546/165; 546/201; 544/130; 546/189; 514/329; 514/326; 514/318; 514/314; 514/323; 514/316; 436/501

(57) ABSTRACT

The invention relates to amide compounds of Formula I: (I) and pharmaceutically acceptable salts, prodrugs and solvates thereof, wherein: Y is CO or SOm; Z is each optionally substituted lower alkyl, lower alkenyl, cycloalkyl, aryl, heterocyclyl, etc.; R¹ and R² are each independently hydrogen, halogen, cyano, optionally substituted lower alky, optionally substituted cycloalkyl, optionally substituted aryl or optionally substituted heterocyclyl etc. R³ and R⁴ are hydrogen, each optionally substituted lower alkyl, cycloalkyl, aryl or heterocyclyl etc.; X is ═O, optionally substituted lower alkyl, halogen, cyano, nitro etc., n is 0-5, m is 1 or 2 and p is 0-2. The invention is also directed to the use compounds of Formula I to treat, prevent or ameliorate a disorder responsive to the blockade of calcium channels, and particularly N-type calcium channels. Compounds of the present invention are especially useful for treating pain.

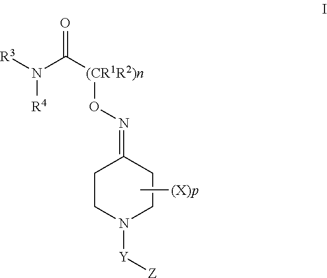

### PRIORITY INFORMATION

This application claims the benefit of U.S. Provisional Patent Application No. 60/924,798, filed May 31, 2007, which is incorporated by reference herein.

### BACKGROUND OF THE INVENTION

1. Field of the Invention

This invention is in the field of medicinal chemistry. The invention relates to amide compounds and the discovery that these compounds act as blockers of calcium (Ca²⁺) channels.

2. Background Art

Calcium ions play fundamental roles in the regulation of many cellular processes. It is therefore essential that their intracellular levels be maintained under strict, yet dynamic control (Davila, H. M., Annals of the New York Academy of Sciences, pp. 102-117 (1999)). Voltage-gated calcium channels (VGCC) serve as one of the important mechanisms for fast calcium influx into the cell. Calcium channels are hetero-oligomeric proteins consisting of a pore-forming subunit (α1), which is able to form functional channels on its own in heterologous expression systems, and a set of auxiliary or regulatory subunits. Calcium channels have been classified based on their pharmacological and/or electrophysiological properties. The classification of voltage-gated calcium channels divides them into three groups: (i) high voltage-activated (HVA) channels, which include L-, N-, P-, and Q-types; (ii) intermediate (IVA) voltage-activated R-type channels; and (iii) low voltage-activated (LVA) T-type channels (Davila, supra). Voltage-gated calcium channels (VGCC) are also known as voltage-dependent calcium channels (VDCC) or voltage-sensitive calcium channels (VSCC).

Voltage-sensitive calcium channels (VSCC) regulate intracellular calcium concentration, which affects various important neuronal functions such as cellular excitability, neurotransmitter release, hormone secretion, intracellular metabolism, neurosecretory activity and gene expression (Hu et al., Bioorganic & Medicinal Chemistry 8:1203-1212 (2000)). N-type channels are found mainly in central and peripheral neurons, being primarily located on presynaptic nerve terminals. These channels regulate the calcium flux required for depolarization-evoked release of a transmitter from synaptic endings. The transmission of pain signals from the periphery to the central nervous system (CNS) is mediated by N-type calcium channels located in the spinal cord (Song et al., J. Med. Chem. 43:3474-3477 (2000)).

The six types of calcium channels (i.e., L, N, P, Q, R, and T) are expressed throughout the nervous system (Wallace, M. S., The Clinical Journal of Pain 16:580-585 (2000)). Voltage-sensitive calcium channels of the N-type exist in the superficial laminae of the dorsal horn and are thought to modulate nociceptive processing by a central mechanism. Blockade of the N-type calcium channel in the superficial dorsal horn modulates membrane excitability and inhibits neurotransmitter release, resulting in pain relief. Wallace (supra) suggests that based on animal models, N-type calcium channel antagonists have a greater analgesic potency than sodium channel antagonists.

N-type calcium channel blockers have usefulness for neuroprotection and analgesia. Ziconotide, which is a selective N-type calcium channel blocker, has been found to have analgesic activity in animal models and neuroprotective activity in focal and global ischemia models (Song et al., supra). Examples of known calcium channel blockers include flunarizine, fluspirilene, cilnipide, PD 157767, SB-201823, SB-206284, NNC09-0026, and PD 151307 (Hu et al., supra).

Blockade of N-type channels can prevent and/or attenuate subjective pain as well as primary and/or secondary hyperalgesia and allodynia in a variety of experimental and clinical conditions (Vanegas, H. et al., Pain 85:9-18 (2000)). N-type voltage-gated calcium channels (VGCC) play a major role in the release of synaptic mediators such as glutamate, acetylcholine, dopamine, norepinephrine, gamma-aminobutyric acid (GABA) and calcitonin gene-related peptide (CGRP).

Inhibition of voltage-gated L-type calcium channels has been shown to be beneficial for neuroprotection (Song et al., supra). However, inhibition of cardiac L-type calcium channels can lead to hypotension. It is believed that a rapid and profound lowering of arterial pressure tends to counteract the neuroprotective effects of L-type calcium channel blockers. A need exists for antagonists that are selective for N-type calcium channels over L-type calcium channels to avoid potential hypotensive effects.

Similar compounds to those of the present application are described in WO98/39325 and GB 1446980, but the structures of these compounds are different from those of the present application.

### BRIEF SUMMARY OF THE INVENTION

The present invention is related to the use of amide compounds represented by Formula I, I′, or I″, below, and the pharmaceutically acceptable salts, prodrugs and solvates thereof, as blockers of calcium (Ca²⁺) channels. Certain compounds of Formula I, I′, or I″ show selectivity as N-type calcium channel blockers.

The invention is also related to treating, preventing or ameliorating a disorder responsive to the blockade of calcium channels in a mammal suffering from excess activity of said channels by administering an effective amount of a compound of Formula I, I′, or I″, or a pharmaceutically acceptable salt, prodrug or solvate thereof, as described herein. Specifically, the invention is related to treating, preventing or ameliorating a disorder responsive to the blockade of N-type calcium channels in a mammal suffering from excess activity of said channels by administering an effective amount of a compound of Formula I, I′, or I″, or a pharmaceutically acceptable salt, prodrug or solvate thereof, as described herein.

One aspect of the present invention is directed to novel compounds of Formula I, or a pharmaceutically acceptable salt, prodrug or solvate thereof.

Another aspect of the present invention is directed to the use of the novel compounds of Formula I, compounds of Formula I′ or compounds of Formula I″, or a pharmaceutically acceptable salt, prodrug or solvate thereof, as blockers of N-type calcium channels.

A further aspect of the present invention is to provide a method for treating, preventing or ameliorating stroke, neuronal damage resulting from head trauma, epilepsy, pain (e.g., acute pain, chronic pain, which includes but is not limited to neuropathic pain and inflammatory pain, or surgical pain), migraine, a mood disorder, schizophrenia, a neurodegenerative disorder (e.g., Alzheimer's disease, amyotrophic lateral sclerosis (ALS), or Parkinson's disease), depression, anxiety, a psychosis, hypertension, or cardiac arrhythmia, by administering an effective amount of a compound of Formula I, I′, or I″, or a pharmaceutically acceptable salt, prodrug or solvate thereof, to a mammal in need of such treatment, prevention or amelioration.

A further aspect of the present invention is to provide a pharmaceutical composition useful for treating, preventing or ameliorating a disorder responsive to the blockade of calcium ion channels, especially N-type calcium ion channels, said pharmaceutical composition containing an effective amount of a compound of Formula I, I′, or I″, or a pharmaceutically acceptable salt, prodrug or solvate thereof, in a mixture with one or more pharmaceutically acceptable carriers.

Also, an aspect of the invention is to provide a method of modulating calcium channels, especially N-type calcium channels, in a mammal, wherein said method comprises administering to the mammal an effective amount of at least one compound of Formula I, I′, or I″, or a pharmaceutically acceptable salt, prodrug or solvate thereof.

A further aspect of the present invention is to provide radiolabeled compounds of Formula I, I′, or I″ and the use of such compounds, or their pharmaceutically acceptable salts, prodrugs or solvates, as radioligands for their binding site on the calcium channel.

A further aspect of the invention is to provide a method for screening a candidate compound for the ability to bind to a receptor using a ³H, ¹¹C or ¹⁴C radiolabeled compound of Formula I, I′, or I″, or a pharmaceutically acceptable salt, prodrug or solvate thereof. This method comprises a) introducing a fixed concentration of the radiolabeled compound to the receptor to form a mixture; b) titrating the mixture with a candidate compound; and c) determining the binding of the candidate compound to said receptor.

A further aspect of the invention is to provide the use of a compound of Formula I, I′, or I″, or a pharmaceutically acceptable salt, prodrug or solvate thereof, in the manufacture of a medicament for treating, preventing or ameliorating stroke, neuronal damage resulting from head trauma, epilepsy, pain, migraine, a mood disorder, schizophrenia, a neurodegenerative disorder, depression, anxiety, a psychosis, hypertension, or cardiac arrhythmia in a mammal. In a preferred embodiment, the invention provides the use of a compound of Formula I, I′, or I″, or a pharmaceutically acceptable salt, prodrug or solvate thereof, in the manufacture of a medicament for treating, preventing or ameliorating acute pain, chronic pain, or surgical pain.

Additional embodiments and advantages of the invention will be set forth in part in the description that follows, and will flow from the description, or may be learned by practice of the invention. The embodiments and advantages of the invention will be realized and attained by means of the elements and combinations particularly pointed out in the appended claims.

It is to be understood that both the foregoing summary and the following detailed description are exemplary and explanatory only and are not restrictive of the invention, as claimed.

### DETAILED DESCRIPTION OF THE INVENTION

One aspect of the present invention is based upon the use of compounds of Formula I, I′, or I″, and the pharmaceutically acceptable salts, prodrugs and solvates thereof, as blockers of Ca²⁺ channels. In view of this property, compounds of Formula I, and the pharmaceutically acceptable salts, prodrugs and solvates thereof, are useful for treating disorders responsive to the blockade of calcium ion channels. In one aspect, compounds of Formula I, I′, or I″, and the pharmaceutically acceptable salts, prodrugs and solvates thereof, selectively block N-type calcium ion channels and, thus, are useful for treating disorders responsive to the selective blockade of N-type calcium ion channels.

The present invention provides

1) A compound having Formula I:

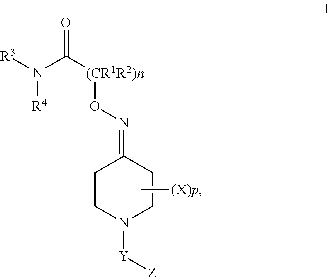

or a pharmaceutically acceptable salt, a prodrug or a solvate thereof, wherein:

Y is CO or SOₘ;

Z is hydrogen, optionally substituted lower alkyl, optionally substituted lower alkenyl, optionally substituted lower alkynyl, optionally substituted cycloalkyl, optionally substituted bicycloalkyl, optionally substituted cycloalkenyl, optionally substituted aryl, optionally substituted heterocyclyl, NR⁵R⁶, OR⁵, SR⁵, COR⁵ or CONR⁵R⁶;

R¹ and R² are each independently hydrogen, halogen, cyano, optionally substituted lower alkyl, optionally substituted lower alkenyl, optionally substituted lower alkynyl, optionally substituted cycloalkyl, optionally substituted cycloalkenyl, optionally substituted aryl or optionally substituted heterocyclyl, or

R¹ and R² taken together, with the carbon atom to which they are attached, form optionally substituted cycloalkane, optionally substituted cycloalkene, optionally substituted bicycloalkane, or optionally substituted heterocycle;

R³ and R⁴ are each independently hydrogen, cyano, optionally substituted lower alkyl, optionally substituted lower alkenyl, optionally substituted lower alkynyl, optionally substituted cycloalkyl, optionally substituted bicycloalkyl, optionally substituted cycloalkenyl, optionally substituted aryl, optionally substituted heterocyclyl, NR⁵R⁶ or OR⁵; or

R³ and R⁴ taken together, with the nitrogen atom to which they are attached, form optionally substituted heterocycle;

R⁵ and R⁶ are each independently hydrogen, optionally substituted lower alkyl, optionally substituted lower alkenyl, optionally substituted lower alkynyl, optionally substituted cycloalkyl, optionally substituted bicycloalkyl, optionally substituted cycloalkenyl, optionally substituted aryl or optionally substituted heterocyclyl,

each X is independently ═O, optionally substituted lower alkyl, optionally substituted lower alkenyl, optionally substituted lower alkynyl, halogen, cyano, nitro, NR⁵R⁶, OR⁵, SR⁵, COR⁵, COOR⁵, CONR⁵R⁶, NR⁵COR⁵, OCOR⁵, SOR⁵, SO₂R⁵, SO₃R⁵, SONR⁵R⁶, SO₂NR⁵R⁶, NRSOR⁵, or NRSO₂R⁵;

n is 0, 1, 2, 3, 4 or 5;

m is 1 or 2; and

p is 0, 1 or 2;

provided that
when n is 1, R³ is NH₂, and R¹, R² and R⁴ are each H, then Y—Z is not COOEt, or
when Y is CO, Z is not optionally substituted (3,4-dihydro-1H-benzo[b]azepin-5(2H)-ylidene)methyl, or

when n is 0, Y is SOm.

2) The compound of the above 1), wherein n is 1.

3) The compound of the above 1) or 2), wherein Y is SO₂.

4) The compound of any one of the above 1) to 3), wherein Z is optionally substituted aryl or optionally substituted heterocyclyl.

5) The compound of any one of the above 1) to 4), wherein R³ is cyano, optionally substituted lower alkyl, optionally substituted cycloalkyl, optionally substituted bicycloalkyl, optionally substituted cycloalkenyl, optionally substituted aryl, optionally substituted heterocyclyl, NR⁵R⁶ or OR⁵, R⁴ is hydrogen, or R³ and R⁴ taken together, with the nitrogen atom to which they are attached, may form heterocyclyl, and R⁵ and R⁶ are each independently hydrogen or optionally substituted lower alkyl.

6) A pharmaceutical composition, comprising the compound of any one of the above 1)-5) and a pharmaceutically acceptable carrier.

7) A method of treating, preventing or ameliorating a disorder responsive to the blockade of calcium channels in a mammal suffering from said disorder, comprising administering to a mammal in need of such treatment, prevention or amelioration an effective amount of a compound of any one of the above 1)-5).

8) The method of the above 7), wherein a disorder responsive to the blockade of N-type calcium channels is treated, prevented or ameliorated.

9) A method for treating, preventing or ameliorating stroke, neuronal damage resulting from head trauma, epilepsy, pain, migraine, a mood disorder, schizophrenia, a neurodegenerative disorder, depression, anxiety, a psychosis, hypertension or cardiac arrhythmia in a mammal, comprising administering an effective amount of a compound of any one of the above 1) -5).

10) The method of the above 9), wherein the method is for treating, preventing or ameliorating pain selected from the group consisting of chronic pain, acute pain, and surgical pain.

11) A method of modulating calcium channels in a mammal, comprising administering to the mammal at least one compound of any one of the above 1) -5).

12) The method of the above 11), wherein the N-type calcium channel is modulated.

13) A compound having Formula I of any of the above 1)-5), wherein the compound is ³H, ¹¹C, or ¹⁴C radiolabeled.

14) A method of screening a candidate compound for the ability to bind to a receptor using a radiolabeled compound of the above 13), comprising a) introducing a fixed concentration of the radiolabeled compound to the receptor to form a mixture; b) titrating the mixture with a candidate compound; and c) determining the binding of the candidate compound to said receptor.

15) Use of a compound of Formula I as claimed in any one of the above 1)-5) in the manufacture of a medicament for the treating, preventing or ameliorating stroke, neuronal damage resulting from head trauma, epilepsy, pain, migraine, a mood disorder, schizophrenia, a neurodegenerative disorder, depression, anxiety, a psychosis, hypertension or cardiac arrhythmia in a mammal.

16) Use of a compound of Formula I as claimed in any one of the above 1)-5) in the manufacture of a medicament for the treating, preventing or ameliorating pain selected from the group consisting of chronic pain, acute pain, and surgical pain.

17) A pharmaceutical composition for modulating calcium channels in a mammal, comprising the compound having Formula I′:

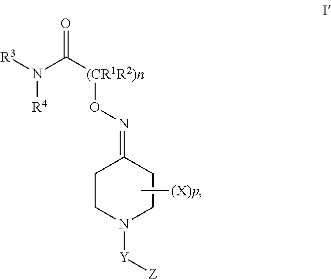

or a pharmaceutically acceptable salt, a prodrug or a solvate thereof, wherein:

Y is CO or SOₘ;

Z is hydrogen, optionally substituted lower alkyl, optionally substituted lower alkenyl, optionally substituted lower alkynyl, optionally substituted cycloalkyl, optionally substituted bicycloalkyl, optionally substituted cycloalkenyl, optionally substituted aryl, optionally substituted heterocyclyl, NR⁵R⁶, OR⁵, SR⁵, COR⁵ or CONR⁵R⁶;

R¹ and R² are each independently hydrogen, halogen, cyano, optionally substituted lower alkyl, optionally substituted lower alkenyl, optionally substituted lower alkynyl, optionally substituted cycloalkyl, optionally substituted cycloalkenyl, optionally substituted aryl or optionally substituted heterocyclyl, or

R¹ and R² taken together, with the carbon atom to which they are attached, form optionally substituted cycloalkane, optionally substituted cycloalkene, optionally substituted bicycloalkane, or optionally substituted heterocycle;

R³ and R⁴ are each independently hydrogen, cyano, optionally substituted lower alkyl, optionally substituted lower alkenyl, optionally substituted lower alkynyl, optionally substituted cycloalkyl, optionally substituted bicycloalkyl, optionally substituted cycloalkenyl, optionally substituted aryl, optionally substituted heterocyclyl, NR⁵R⁶ or OR⁵; or

R³ and R⁴ taken together, with the nitrogen atom to which they are attached, form optionally substituted heterocycle;

R⁵ and R⁶ are each independently hydrogen, optionally substituted lower alkyl, optionally substituted lower alkenyl, optionally substituted lower alkynyl, optionally substituted cycloalkyl, optionally substituted bicycloalkyl, optionally substituted cycloalkenyl, optionally substituted aryl or optionally substituted heterocyclyl;

each X is independently ═O, optionally substituted lower alkyl, optionally substituted lower alkenyl, optionally substituted lower alkynyl, halogen, cyano, nitro, NR⁵R⁶, OR⁵, SR⁵, COR⁵, COOR⁵, CONR⁵R⁶, NR⁵COR⁵, OCOR⁵, SOR⁵, SO₂R⁵, SO₃R⁵, SONR⁵R⁶, SO₂NR⁵R⁶, NRSOR⁵, or NRSO₂R⁵;

n is 0, 1, 2, 3, 4 or 5;

m is 1 or 2; and

p is 0, 1 or 2;

and a pharmaceutically acceptable carrier.

18) A method of treating, preventing or ameliorating a disorder responsive to the blockade of calcium channels in a mammal suffering from said disorder, comprising administering to a mammal in need of such treatment, prevention or amelioration an effective amount of a compound of the above 17).

19) The method of the above 18), wherein a disorder responsive to the blockade of N-type calcium channels is treated, prevented or ameliorated.

20) A method for treating, preventing or ameliorating stroke, neuronal damage resulting from head trauma, epilepsy, pain, migraine, a mood disorder, schizophrenia, a neurodegenerative disorder, depression, anxiety, a psychosis, hypertension or cardiac arrhythmia in a mammal, comprising administering an effective amount of a compound of the above 17).

21) The method of the above 20), wherein the method is for treating, preventing or ameliorating pain selected from the group consisting of chronic pain, acute pain, and surgical pain.

22) A method of modulating calcium channels in a mammal, comprising administering to the mammal at least one compound the above 17).

23) The method of the above 22), wherein the N-type calcium channel is modulated.

24) A method of screening a candidate compound for the ability to bind to a receptor using a radiolabeled compound of the above 17), comprising a) introducing a fixed concentration of the radiolabeled compound to the receptor to form a mixture; b) titrating the mixture with a candidate compound; and c) determining the binding of the candidate compound to said receptor.

25) Use of a compound of the above 17) in the manufacture of a medicament for the treating, preventing or ameliorating stroke, neuronal damage resulting from head trauma, epilepsy, pain, migraine, a mood disorder, schizophrenia, a neurodegenerative disorder, depression, anxiety, a psychosis, hypertension or cardiac arrhythmia in a mammal.

26) Use of a compound of the above 17) in the manufacture of a medicament for the treating, preventing or ameliorating pain selected from the group consisting of chronic pain,acute pain, and surgical pain.

27) A kit comprising a container containing an effective amount of the compound or a pharmaceutically acceptable derivative of the compound of the above 1).

In the present specification, the term “halogen” includes fluorine, chlorine, bromine and iodine. Fluorine or chlorine is preferable. The halogen parts of “halo(lower)alkyl”, “halo(lower)alkoxy” and “haloacyl” are the same as the above “halogen”.

The term “lower alkyl” includes straight or branched chain alkyl having 1 to 10 carbon atoms, preferably 1 to 6 carbon atoms and most preferably 1 to 3 carbon atoms. For example, included are methyl, ethyl, n-propyl, isopropyl, n-butyl, isobutyl, sec-butyl, tert-butyl, n-pentyl, isopentyl, neopentyl, hexyl, isohexyl, n-heptyl, isoheptyl, n-octyl, isooctyl, n-nonyl, n-decyl and the like.

The optional substituents in “optionally substituted lower alkyl”, include
1) halogen,
2) hydroxy,
3) carboxy,
4) mercapto,
5) cyano,
5) lower alkoxy optionally substituted with at least one substituent selected from the group consisting of Group A and Group C,
6) acyl optionally substituted with at least one substituent selected from the group consisting of Group A, Group B and Group C,
7) acyloxy optionally substituted with at least one substituent selected from the group consisting of Group A, Group B and Group C
8) lower alkoxycarbonyl optionally substituted with at least one substituent selected from the group consisting of Group A and Group C,
9) aryloxycarbonyl optionally substituted with at least one substituent selected from the group consisting of Group A, Group B and Group C,
10) lower alkylthio optionally substituted with at least one substituent selected from the group consisting of Group A and Group C,
11) lower alkylsulfonyl optionally substituted with at least one substituent selected from the group consisting of Group A and Group C,
12) amino optionally substituted with at least one substituent selected from the group consisting of Group A, Group B and Group C
13) imino optionally substituted with at least one substituent selected from the group consisting of Group A, Group B and Group C,
14) carbamoyl optionally substituted with at least one substituent selected from the group consisting of Group B and Group C,
15) carbamoyloxy optionally substituted with at least one substituent selected from the group consisting of Group B and Group C,
16) thiocarbamoyl optionally substituted with at least one substituent selected from the group consisting of Group B and Group C,
17) cycloalkyl optionally substituted with at least one substituent selected from the group consisting of Group A, Group B and Group C,
18) cycloalkenyl optionally substituted with at least one substituent selected from the group consisting of Group A, Group B and Group C,
19) aryl optionally substituted with at least one substituent selected from the group consisting of Group A, Group B and Group C,
20) heterocyclyl optionally substituted with at least one substituent selected from the group consisting of Group A, Group B, Group C and oxo,
21) aryloxy optionally substituted with at least one substituent selected from the group consisting of Group A, Group B and Group C,
22) arylthio optionally substituted with at least one substituent selected from the group consisting of Group A, Group B and Group C,
23) cycloalkylsulfonyl optionally substituted with at least one substituent selected from the group consisting of Group A, Group B and Group C,
24) arylsulfonyl optionally substituted with at least one substituent selected from the group consisting of Group A, Group B and Group C
25) heterocyclylsulfonyl optionally substituted with at least one substituent selected from the group consisting of Group A, Group B, Group C, and oxo
   and the like.

Group A includes hydroxy, halogen, lower alkoxy, halo(lower)alkoxy, hydroxy(lower)alkoxy, aryl(lower)alkoxy, acyl, haloacyl, aminoacyl, acyloxy, carboxy, lower alkoxycarbonyl, carbamoyl, lower alkylcarbamoyl, and optionally substituted amino, wherein the substituents are selected from the group consisting of halogen, hydroxy, lower alkyl, hydroxy(lower)alkyl, lower alkoxy(lower)alkyl, acyl, cycloalkyl, aryl and heterocyclyl.

Group B includes lower alkyl, halo(lower)alkyl, hydroxy(lower)alkyl, lower alkoxy(lower)alkyl, amino(lower)alkyl, lower alkylamino(lower)alkyl, aryl(lower)alkyl and heterocyclyl(lower)alkyl.

Group C includes optionally substituted cycloalkyl, optionally substituted cycloalkenyl, optionally substituted aryl and optionally substituted heterocyclyl, wherein the substituents are selected from the group consisting of Group A, Group B and oxo.

The term “optionally substituted lower alkyl” refers to a lower alkyl that can be substituted with one or more of the above-mentioned substituents at any possible positions.

The lower alkyl parts of “lower alkoxy”, “lower alkoxycarbonyl”, “lower alkylsulfonyl”, “lower alkylthio”, “halo(lower)alkyl”, “hydroxy(lower)alkyl”, “amino(lower)alkyl”, “lower alkylamino”, “lower alkylamino(lower)alkyl”, “aryl(lower)alkyl”, “halo(lower)alkoxy”, “hydroxy(lower)alkoxy”, “lower alkoxy(lower)alkyl”, “aryl(lower)alkoxy”, “lower alkylcarbamoyl”, “heterocyclyl(lower)alkyl”, and “lower alkylenedioxy” are as defined for “lower alkyl”.

The optional substituents in “optionally substituted lower alkoxy” include those defined for “optionally substituted lower alkyl”.

The term “lower alkenyl” refers to straight or branched chain alkenyl of 2 to 10 carbon atoms, preferably 2 to 8 carbon atoms, more preferably 3 to 6 carbon atoms having at least one double bond at any possible positions. For example, useful lower alkenyl groups include vinyl, propenyl, isopropenyl, butenyl, isobutenyl, prenyl, butadienyl, pentenyl, isopentenyl, pentadienyl, hexenyl, isohexenyl, hexadienyl, heptenyl, octenyl, nonenyl, decenyl and the like.

The optional substituents in “optionally substituted lower alkenyl” and “optionally substituted lower alkynyl” include those defined for “optionally substituted lower alkyl”.

The term “lower alkynyl” refers to straight or branched chain alkenyl of 2 to 10 carbon atoms, preferably 2 to 8 carbon atoms, more preferably 3 to 6 carbon atoms having at least one triple bond at any possible positions. Furthermore, “lower alkynyl” can have at least one double bond at any possible positions. Useful lower alkynyl groups are, for example, ethynyl, propynyl, butynyl, pentynyl, hexynyl, heptynyl, octynyl, nonynyl, decynyl and the like.

Substituents for “optionally substituted lower alkynyl” are those defined for “optionally substituted lower alkyl”.

The term “acyl” refers to straight or branched chain aliphatic acyl having 1 to 10 carbon atoms, preferably 1 to 6 carbon atoms, more preferably 1 to 4 carbon atoms, cyclic aliphatic acyl having 4 to 9 carbon atoms, preferably 4 to 7 carbon atoms, aroyl and heterocyclylcarbonyl. Useful acyl groups include, for example, formyl, acetyl, propionyl, butyryl, isobutyryl, valeryl, pivaloyl, hexanoyl, acryloyl, propioloyl, methacryloyl, crotonoyl, cyclopropylcarbonyl, cyclohexylcarbonyl, cyclooctylcarbonyl, benzoyl, pyridinecarbonyl, pyrimidinecarbonyl, piperidincarbonyl, piperazinocarbonyl, morphorinocarbonyl and the like.

The acyl part in “acyloxy”, “haloacyl” and “aminoacyl” is that defined for “acyl”.

The term “cycloalkyl” refers to a carbocycle having 3 to 8 carbon atoms, and includes cyclopropyl, cyclobutyl, cyclopentyl, cyclohexyl, cycloheptyl, cyclooctyl and the like.

Examples of substituents for “optionally substituted cycloalkyl” are
1) lower alkyl optionally substituted with at least one substituent selected from the group consisting of Group A and Group C, and
2) the same as those defined for the above “optionally substituted lower alkyl”.

The term “optionally substituted cycloalkyl” refers to a cycloalkyl defined above that can be substituted with one or more of these substituents.

The cycloalkyl part of “cycloalkylsulfonyl” is as defined for “cycloalkyl”.

The term “bicycloalkyl” refers to a bicyclic hydrocarbon ring system having from 6 to 14 carbon atoms and at least one saturated cyclic alkyl ring. Examples of bicycloalkyl groups are indanyl, norbornyl, 1,2,3,4-tetrahydronaphthyl, 5,6,7,8-tetrahydronaphthyl, perhydronaphthyl, and the like.

Examples of substituents for “optionally substituted bicycloalkyl” are the same as those defined for the above “optionally substituted cycloalkyl.”

The term “cycloalkenyl” refers to a group having at least one double bond at any possible positions in the above “cycloalkyl”. Examples are cyclopropenyl, cyclobutenyl, cyclopentenyl, cyclohexenyl and cyclohexadienyl. Substituents for “optionally substituted cycloalkenyl” are those defined for “optionally substituted cycloalkyl.”

The optional substituents in “optionally substituted amino” include
1) lower alkyl optionally substituted with at least one substituent selected from the group consisting of Group A and Group C, and
2) the same as those defined for the above “optionally substituted lower alkyl”.
   The term “lower alkylamino” includes mono-alkylamino and di-alkylamino.

The term “aryl” includes phenyl, naphthyl, anthryl, phenanthryl, indenyl and the like. Phenyl is preferable.

The aryl part in “aryloxy”, “aryloxycarbonyl”, “arylthio”, “arylsulfonyl”, “aryl(lower)alkyl”, and “aryl(lower)alkoxy” is that defined for “aryl”.

The terms “heterocyclyl” or “heterocycle” refers to a heterocyclic group containing at least one heteroatom arbitrarily selected from the group consisting of O, S and N. Useful heterocyclyl groups are, for example, 5- or 6-membered heteroaryl such as pyrrolyl, imidazolyl, pyrazolyl, pyridyl, pyridazinyl, pyrimidinyl, pyrazinyl, isoxazolyl, oxazolyl, oxadiazolyl, isothiazolyl, thiazolyl, thiadiazolyl, furyl and thienyl; fused heterocyclyl groups having two rings, such as indolyl, isoindolyl, indazolyl, indolizinyl, indolinyl, isoindolinyl, quinolyl, isoquinolyl, cinnolinyl, phthalazinyl, quinazolinyl, naphthyridinyl, quinoxalinyl, purinyl, pteridinyl, benzopyranyl, benzimidazolyl, benzisoxazolyl, benzoxazolyl, benzoxadiazolyl, benzisothiazolyl, benzothiazolyl, benzothiadiazolyl, benzofuryl, isobenzofuryl, benzothienyl, benzotriazolyl, imidazopyridyl, triazoropyridyl, imidazothiazolyl, pyrazinopyridazinyl, quinazolinyl, quinolyl, isoquinolyl, naphthyridinyl, dihydropyridyl, tetrahydroquinolyl and tetrahydrobenzothienyl; fused heterocyclyl groups having three rings, such as carbazolyl, acridinyl, xanthenyl, phenothiazinyl, phenoxathiinyl, phenoxazinyl and dibenzofuryl; and non-aromatic heterocyclyl such as dioxanyl, thiiranyl, oxiranyl, oxathiolanyl, azetidinyl, thianyl, pyrrolidinyl, pyrrolinyl, imidazolidinyl, imidazolinyl, pyrazolidinyl, pyrazolinyl, piperidyl, piperazinyl, morpholinyl, morpholino, thiomorpholinyl, thiomorpholino, dihydropyridyl, tetrahydrofuryl, tetrahydropyranyl, tetrahydrothiazolyl and tetrahydroisothiazolyl.

The heterocyclyl in “heterocyclyl(lower)alkyl” and “heterocyclylsulfonyl” is that defined for “heterocyclyl”.

Examples of the substituents in “optionally substituted aryl” and “optionally substituted heterocyclyl” include
1) the same as those defined for the above “optionally substituted lower alkyl”,
2) lower alkyl optionally substituted with at least one substituent selected from the group consisting of Group A and Group C,
3) oxo and
4) lower alkylenedioxy.
   These substituents can attach to one or more of any possible positions.

The phrase “R¹ and R² taken together, with the carbon atom to which they are attached, form optionally substituted cycloalkane, optionally substituted cycloalkene, optionally substituted bicycloalkane, or optionally substituted heterocycle” means

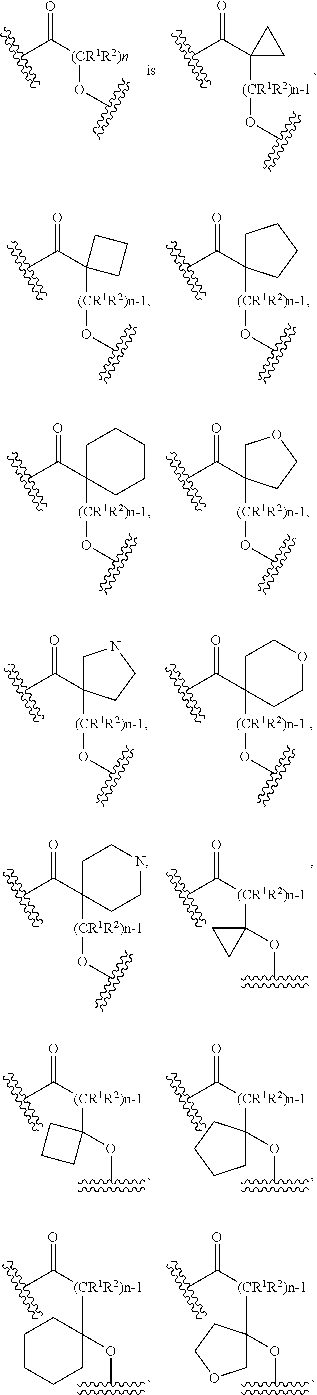

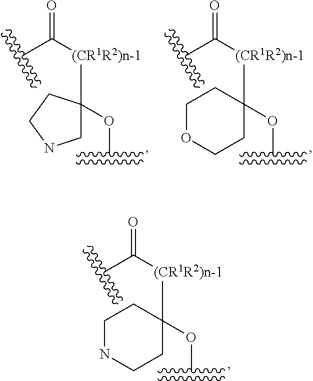

or the like. These cycloalkane, cycloalkene, bicycloalkane, and heterocycle can be substituted with the substituents described above for “optionally substituted cycloalkyl”, “optionally substituted cycloalkenyl”, “optionally substituted bicycloalkyl”, and “optionally substituted heterocyclyl”, respectively.

The phrase “R³ and R⁴ taken together, with the nitrogen atom to which they are attached, form optionally substituted heterocycle” means

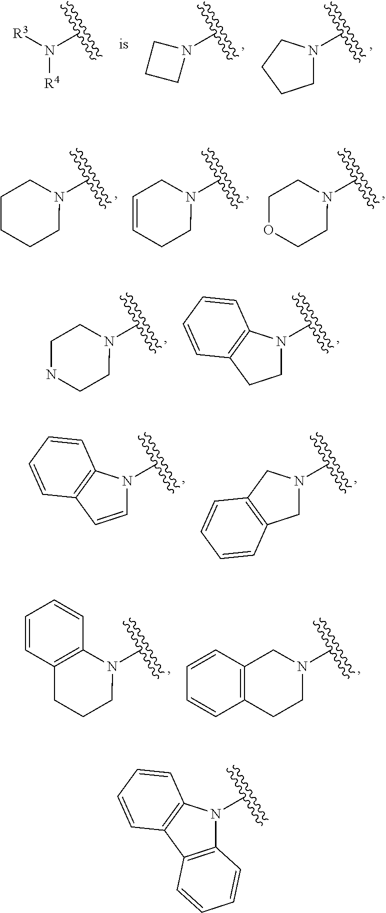

or the like. These heterocycle groups can be substituted with the substituents described above for “optionally substituted heterocyclyl”.

When p is 2, each X can be the same or different.

The term “optionally substituted (3,4-dihydro-1H-benzo[b]azepin-5(2H)-ylidene)methyl” includes

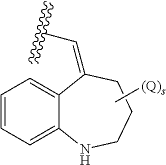

wherein Q is a substituent and s is an integer of 0 to 7. Examples are

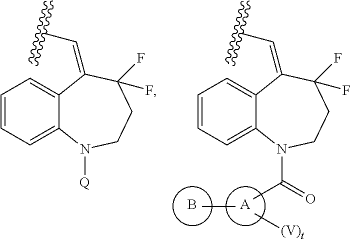

and the like,

wherein Q is a substituent, A is 5-membered heteroarylene, preferably thiazolyl, B is an optionally substituted aryl or optionally substituted 5 or 6-membered heteroaryl, preferably phenyl, V is lower alkyl, lower alkoxy, lower alkylthio or lower alkylcarbonyl, each of which can be substituted with halogen, and t is 0, 1 or 2.

In one embodiment, preferable Amide Compounds are the compounds of the following Formula I″:

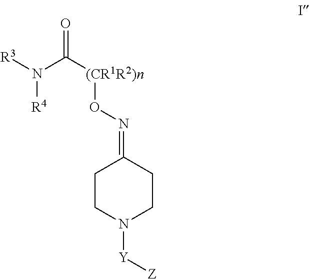

wherein —NR³R⁴ is selected from the group consisting of

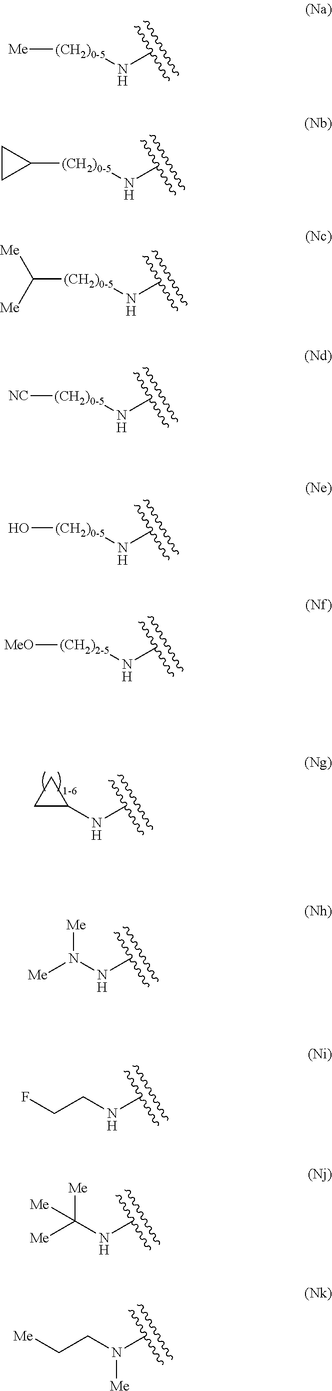

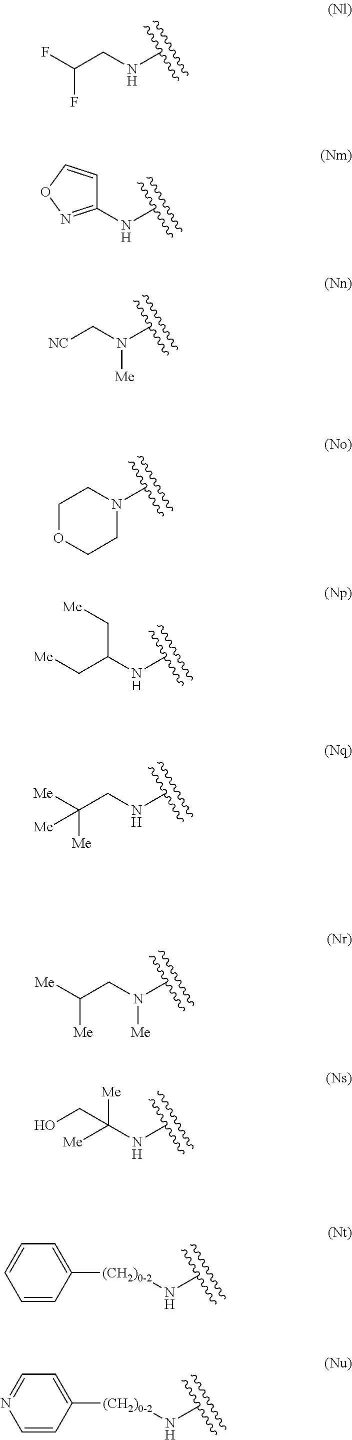

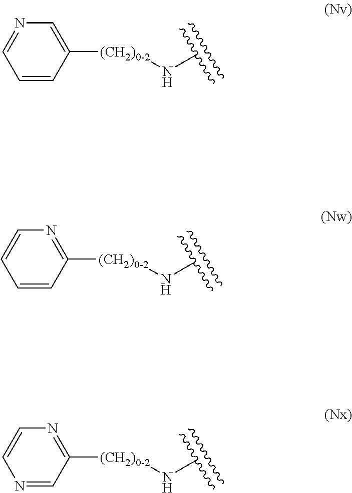

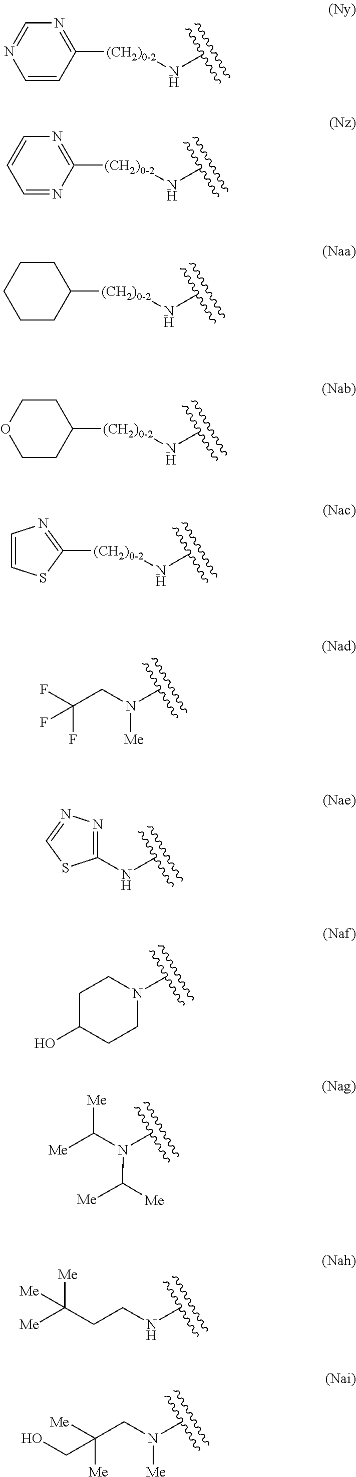

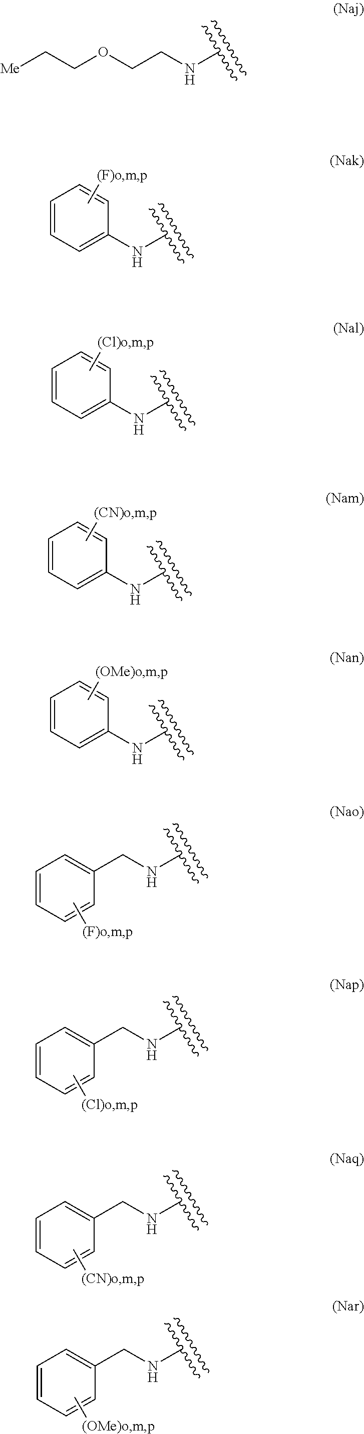

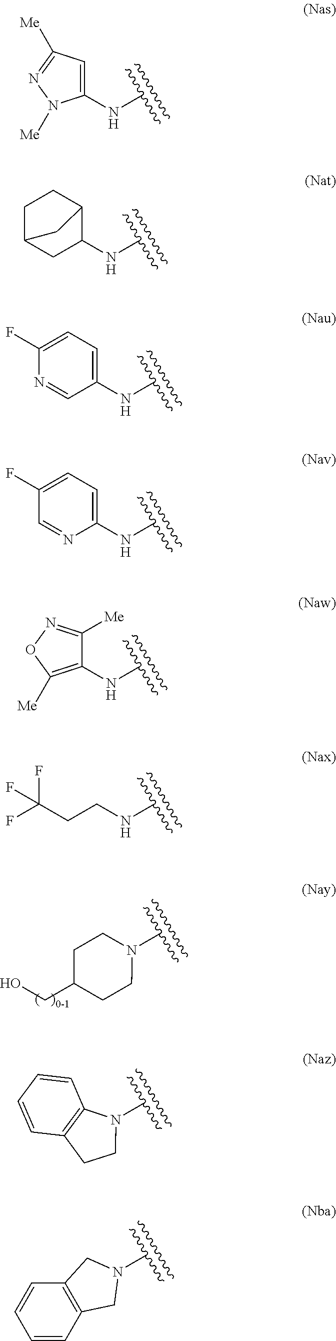

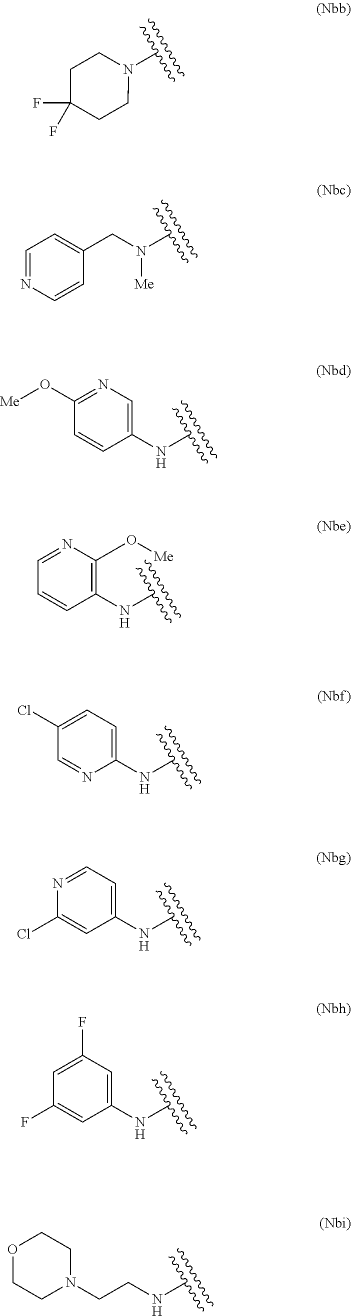

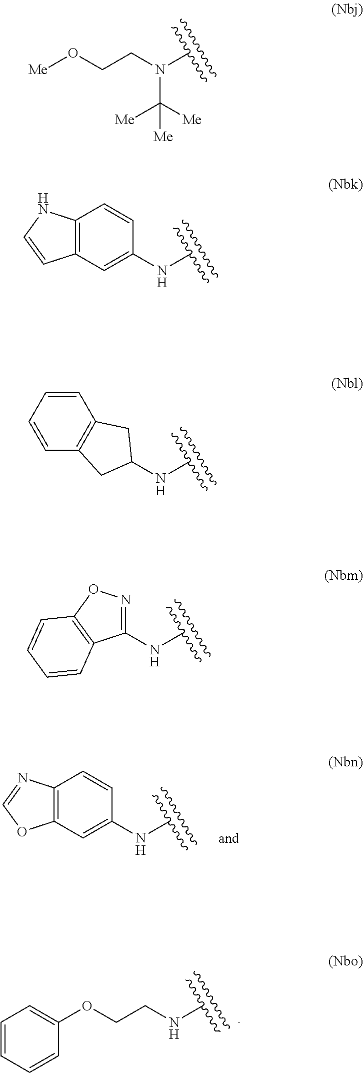

In another embodiment, useful compounds of Formula I″ include those wherein —(CR¹R²)n- is selected from the group consisting of

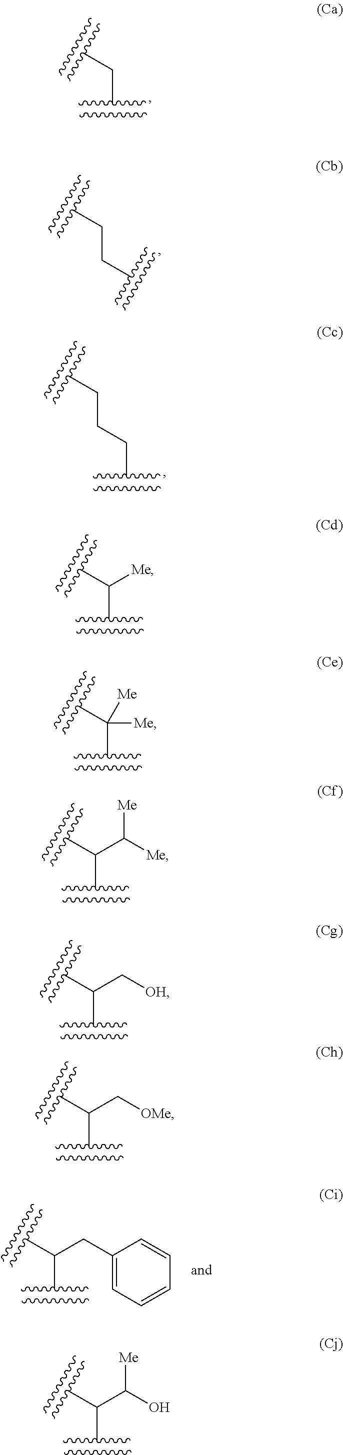

In another embodiment, useful compounds of Formula I″ include those wherein Y—Z is selected from the group consisting of:

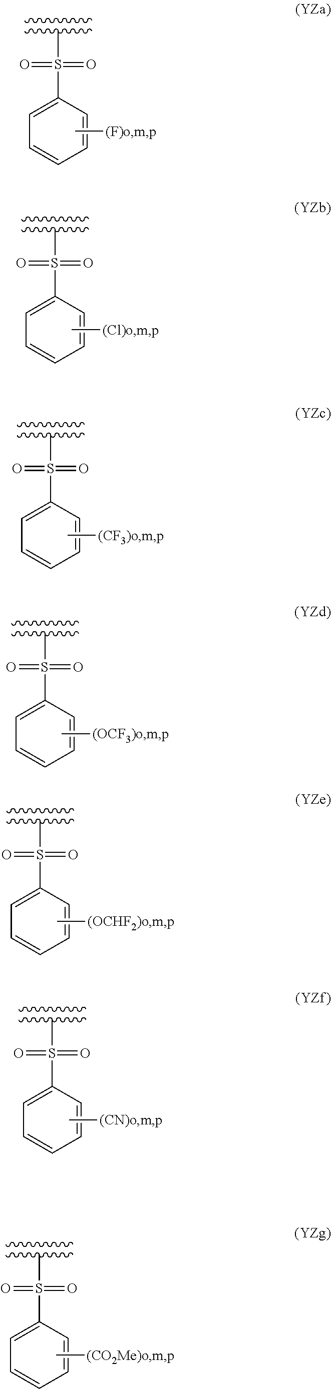

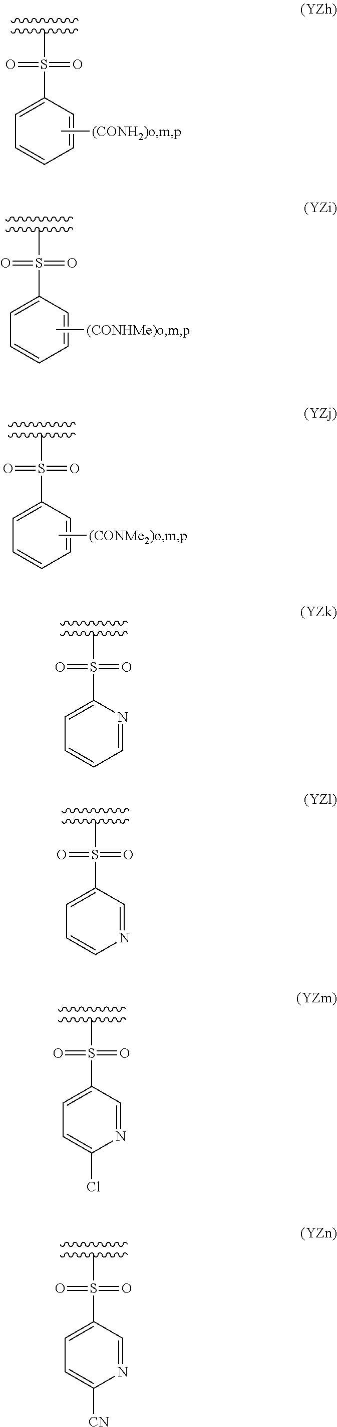

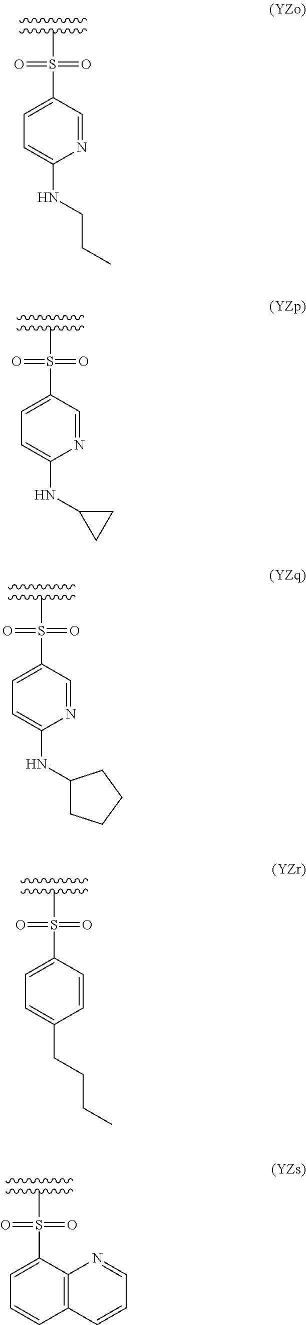

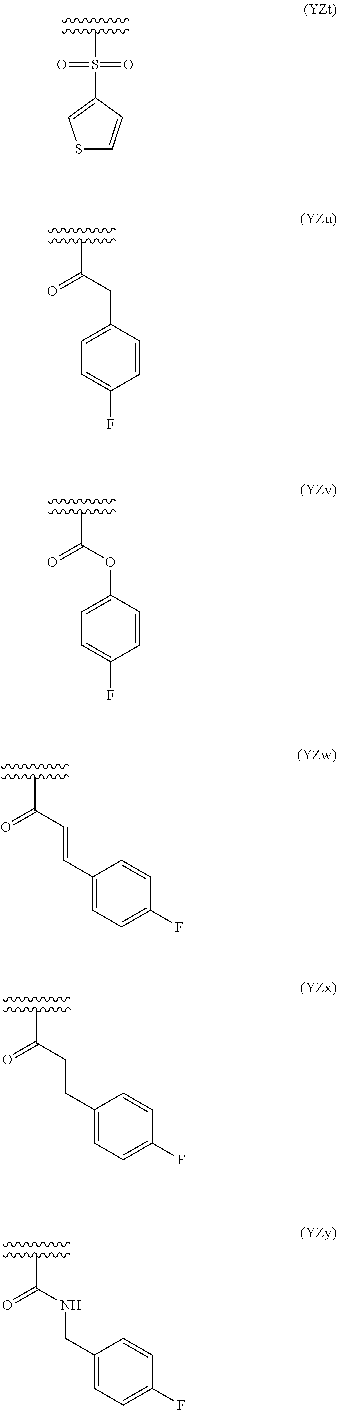

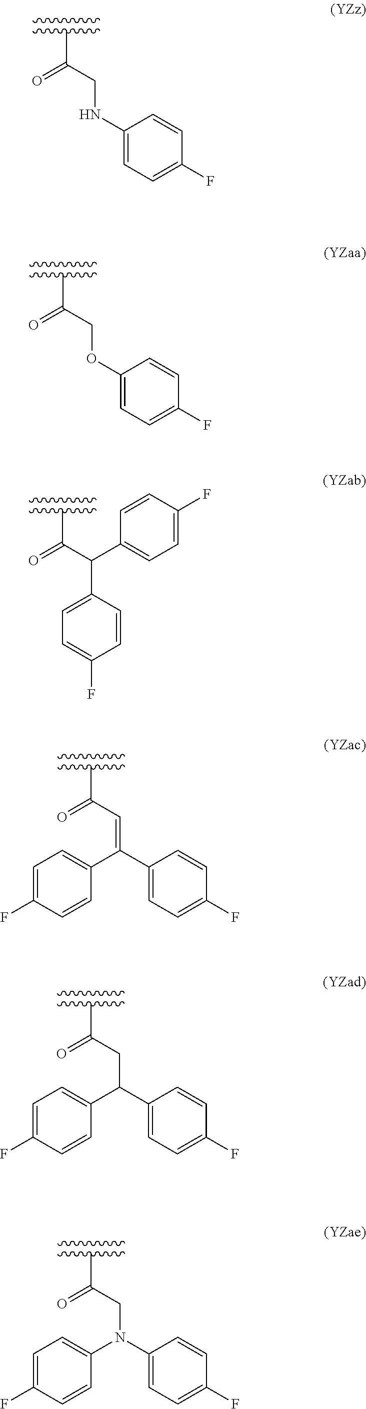

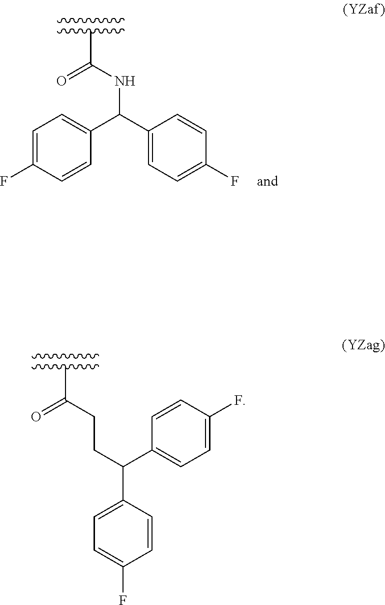

In another embodiment, useful compounds of Formula I″ include those wherein the combination of —NR³R⁴, —(CR¹R²)n- and —Y—Z(—NR³R⁴, —(CR¹R²)n-, —Y—Z) is selected from the group consisting of:

(Na,Ca,YZa),(Na,Ca,YZb),(Na,Ca,YZc),(Na,Ca,YZd),(Na,Ca,YZe),(Na,Ca,YZf),(Na,Ca,YZ g),(Na,Ca,YZh),(Na,Ca,YZi),(Na,Ca,YZj),(Na,Ca,YZk),(Na,Ca,YZl),(Na,Ca,YZm),(Na,Ca, YZn),(Na,Ca,YZo),(Na,Ca,YZp),(Na,Ca,YZq),(Na,Ca,YZr),(Na,Ca,YZs),(Na,Ca,YZt),(Na,C a,YZu),(Na,Ca,YZv),(Na,Ca,YZw),(Na,Ca,YZx),(Na,Ca,YZy),(Na,Ca,YZz),(Na,Ca,YZaa),( Na,Ca,YZab),(Na,Ca,YZac),(Na,Ca,YZad),(Na,Ca,YZae),(Na,Ca,YZaf),(Na,Ca,YZag),(Na, Cb,YZa),(Na,Cb,YZb),(Na,Cb,YZc),(Na,Cb,YZd),(Na,Cb,YZe),(Na,Cb,YZf),(Na,Cb,YZg),( Na,Cb,YZh),(Na,Cb,YZi),(Na,Cb,YZj),(Na,Cb,YZk),(Na,Cb,YZl),(Na,Cb,YZm),(Na,Cb,YZ n),(Na,Cb,YZo),(Na,Cb,YZp),(Na,Cb,YZq),(Na,Cb,YZr),(Na,Cb,YZs),(Na,Cb,YZt),(Na,Cb, YZu),(Na,Cb,YZv),(Na,Cb,YZw),(Na,Cb,YZx),(Na,Cb,YZy),(Na,Cb,YZz),(Na,Cb,YZaa),(N a,Cb,YZab),(Na,Cb,YZac),(Na,Cb,YZad),(Na,Cb,YZae),(Na,Cb,YZaf),(Na,Cb,YZag),(Na,C c,YZa),(Na,Cc,YZb),(Na,Cc,YZc),(Na,Cc,YZd),(Na,Cc,YZe),(Na,Cc,YZf),(Na,Cc,YZg),(Na ,Cc,YZh),(Na,Cc,YZi),(Na,Cc,YZj),(Na,Cc,YZk),(Na,Cc,YZl),(Na,Cc,YZm),(Na,Cc,YZn),( Na,Cc,YZo),(Na,Cc,YZp),(Na,Cc,YZq),(Na,Cc,YZr),(Na,Cc,YZs),(Na,Cc,YZt),(Na,Cc,YZu) ,(Na,Cc,YZv),(Na,Cc,YZw),(Na,Cc,YZx),(Na,Cc,YZy),(Na,Cc,YZz),(Na,Cc,YZaa),(Na,Cc, YZab),(Na,Cc,YZac),(Na,Cc,YZad),(Na,Cc,YZae),(Na,Cc,YZaf),(Na,Cc,YZag),(Na,Cd,YZa) ,(Na,Cd,YZb),(Na,Cd,YZc),(Na,Cd,YZd),(Na,Cd,YZe),(Na,Cd,YZf),(Na,Cd,YZg),(Na,Cd, YZh),(Na,Cd,YZi),(Na,Cd,YZj),(Na,Cd,YZk),(Na,Cd,YZl),(Na,Cd,YZm),(Na,Cd,YZn),(Na, Cd,YZo),(Na,Cd,YZp),(Na,Cd,YZq),(Na,Cd,YZr),(Na,Cd,YZs),(Na,Cd,YZt),(Na,Cd,YZu),( Na,Cd,YZv),(Na,Cd,YZw),(Na,Cd,YZx),(Na,Cd,YZy),(Na,Cd,YZz),(Na,Cd,YZaa),(Na,Cd,Y Zab),(Na,Cd,YZac),(Na,Cd,YZad),(Na,Cd,YZae),(Na,Cd,YZaf),(Na,Cd,YZag),(Na,Ce,YZa), (Na,Ce,YZb),(Na,Ce,YZc),(Na,Ce,YZd),(Na,Ce,YZe),(Na,Ce,YZf),(Na,Ce,YZg),(Na,Ce,YZ h),(Na,Ce,YZi),(Na,Ce,YZj),(Na,Ce,YZk),(Na,Ce,YZl),(Na,Ce,YZm),(Na,Ce,YZn),(Na,Ce, YZo),(Na,Ce,YZp),(Na,Ce,YZq),(Na,Ce,YZr),(Na,Ce,YZs),(Na,Ce,YZt),(Na,Ce,YZu),(Na,C e,YZv),(Na,Ce,YZw),(Na,Ce,YZx),(Na,Ce,YZy),(Na,Ce,YZz),(Na,Ce,YZaa),(Na,Ce,YZab),( Na,Ce,YZac),(Na,Ce,YZad),(Na,Ce,YZae),(Na,Ce,YZaf),(Na,Ce,YZag),(Na,Cf,YZa),(Na,Cf, YZb),(Na,Cf,YZc),(Na,Cf,YZd),(Na,Cf,YZe),(Na,Cf,YZf),(Na,Cf,YZg),(Na,Cf,YZh),(Na,Cf ,YZi),(Na,Cf,YZj),(Na,Cf,YZk),(Na,Cf,YZl),(Na,Cf,YZm),(Na,Cf,YZn),(Na,Cf,YZo),(Na,Cf ,YZp),(Na,Cf,YZq),(Na,Cf,YZr),(Na,Cf,YZs),(Na,Cf,YZt),(Na,Cf,YZu),(Na,Cf,YZv),(Na,Cf ,YZw),(Na,Cf,YZx),(Na,Cf,YZy),(Na,Cf,YZz),(Na,Cf,YZaa),(Na,Cf,YZab),(Na,Cf,YZac),(N a,Cf,YZad),(Na,Cf,YZae),(Na,Cf,YZaf),(Na,Cf,YZag),(Na,Cg,YZa),(Na,Cg,YZb),(Na,Cg,Y Zc),(Na,Cg,YZd),(Na,Cg,YZe),(Na,Cg,YZf),(Na,Cg,YZg),(Na,Cg,YZh),(Na,Cg,YZi),(Na,Cg ,YZj),(Na,Cg,YZk),(Na,Cg,YZl),(Na,Cg,YZm),(Na,Cg,YZn),(Na,Cg,YZo),(Na,Cg,YZp),(Na ,Cg,YZq),(Na,Cg,YZr),(Na,Cg,YZs),(Na,Cg,YZt),(Na,Cg,YZu),(Na,Cg,YZv),(Na,Cg,YZw), (Na,Cg,YZx),(Na,Cg,YZy),(Na,Cg,YZz),(Na,Cg,YZaa),(Na,Cg,YZab),(Na,Cg,YZac),(Na,Cg ,YZad),(Na,Cg,YZae),(Na,Cg,YZaf),(Na,Cg,YZag),(Na,Ch,YZa),(Na,Ch,YZb),(Na,Ch,YZc), (Na,Ch,YZd),(Na,Ch,YZe),(Na,Ch,YZf),(Na,Ch,YZg),(Na,Ch,YZh),(Na,Ch,YZi),(Na,Ch,YZ j),(Na,Ch,YZk),(Na,Ch,YZl),(Na,Ch,YZm),(Na,Ch,YZn),(Na,Ch,YZo),(Na,Ch,YZp),(Na,Ch, YZq),(Na,Ch,YZr),(Na,Ch,YZs),(Na,Ch,YZt),(Na,Ch,YZu),(Na,Ch,YZv),(Na,Ch,YZw),(Na, Ch,YZx),(Na,Ch,YZy),(Na,Ch,YZz),(Na,Ch,YZaa),(Na,Ch,YZab),(Na,Ch,YZac),(Na,Ch,YZ ad),(Na,Ch,YZae),(Na,Ch,YZaf),(Na,Ch,Yzag),(Na,Ci,YZa),(Na,Ci,YZb),(Na,Ci,YZc),(Na,C i,YZd),(Na,Ci,YZe),(Na,Ci,YZf),(Na,Ci,YZg),(Na,Ci,YZh),(Na,Ci,YZi),(Na,Ci,YZj),(Na,Ci, YZk),(Na,Ci,YZl),(Na,Ci,YZm),(Na,Ci,YZn),(Na,Ci,YZo),(Na,Ci,YZp),(Na,Ci,YZq),(Na,Ci, YZr),(Na,Ci,YZs),(Na,Ci,YZt),(Na,Ci,YZu),(Na,Ci,YZv),(Na,Ci,YZw),(Na,Ci,YZx),(Na,Ci, YZy),(Na,Ci,YZz),(Na,Ci,YZaa),(Na,Ci,YZab),(Na,Ci,YZac),(Na,Ci,YZad),(Na,Ci,YZae),( Na,Ci,YZaf),(Na,Ci,Yzag),(Na,Cj,YZa),(Na,Cj,YZb),(Na,Cj,YZc),(Na,Cj,YZd),(Na,Cj,YZe), (Na,Cj,Ya),(Na,Cj,YZg),(Na,Cj,YZh),(Na,Cj,YZi),(Na,Cj,YZj),(Na,Cj,YZk),(Na,Cj,YZl),( Na,Cj,YZm),(Na,Cj,YZn),(Na,Cj,YZo),(Na,Cj,YZp),(Na,Cj,YZq),(Na,Cj,YZr),(Na,Cj,YZs),( Na,Cj,YZt),(Na,Cj,YZu),(Na,Cj,YZv),(Na,Cj,YZw),(Na,Cj,YZx),(Na,Cj,YZy),(Na,Cj,YZz),( Na,Cj,YZaa),(Na,Cj,YZab),(Na,Cj,YZac),(Na,Cj,YZad),(Na,q,YZae),(Na,Cj,YZaf),(Na,Cj, Yzag),(Nb,Ca,YZa),(Nb,Ca,YZb),(Nb,Ca,YZc),(Nb,Ca,YZd),(Nb,Ca,YZe),(Nb,Ca,YZf),(Nb ,Ca,YZg),(Nb,Ca,YZh),(Nb,Ca,YZi),(Nb,Ca,YZj),(Nb,Ca,YZk),(Nb,Ca,YZl),(Nb,Ca,YZm),( Nb,Ca,YZn),(Nb,Ca,YZo),(Nb,Ca,YZp),(Nb,Ca,YZq),(Nb,Ca,YZr),(Nb,Ca,YZs),(Nb,Ca,YZ t),(Nb,Ca,YZu),(Nb,Ca,YZv),(Nb,Ca,YZw),(Nb,Ca,YZx),(Nb,Ca,YZy),(Nb,Ca,YZz),(Nb,Ca ,YZaa),(Nb,Ca,YZab),(Nb,Ca,YZac),(Nb,Ca,YZad),(Nb,Ca,YZae),(Nb,Ca,YZaf),(Nb,Ca,YZ ag),(Nb,Cb,YZa),(Nb,Cb,YZb),(Nb,Cb,YZc),(Nb,Cb,YZd),(Nb,Cb,YZe),(Nb,Cb,YZf),(Nb,C b,YZg),(Nb,Cb,YZh),(Nb,Cb,YZi),(Nb,Cb,YZj),(Nb,Cb,YZk),(Nb,Cb,YZl),(Nb,Cb,YZm),( Nb,Cb,YZn),(Nb,Cb,YZo),(Nb,Cb,YZp),(Nb,Cb,YZq),(Nb,Cb,YZr),(Nb,Cb,YZs),(Nb,Cb,Y Zt),(Nb,Cb,YZu),(Nb,Cb,YZv),(Nb,Cb,YZw),(Nb,Cb,YZx),(Nb,Cb,YZy),(Nb,Cb,YZz),(Nb, Cb,YZaa),(Nb,Cb,YZab),(Nb,Cb,YZac),(Nb,Cb,YZad),(Nb,Cb,YZae),(Nb,Cb,YZaf),(Nb,Cb, YZag),(Nb,Cc,YZa),(Nb,Cc,YZb),(Nb,Cc,YZc),(Nb,Cc,YZd),(Nb,Cc,YZe),(Nb,Cc,YZf),(Nb ,Cc,YZg),(Nb,Cc,YZh),(Nb,Cc,YZi),(Nb,Cc,YZj),(Nb,Cc,YZk),(Nb,Cc,YZl),(Nb,Cc,YZm),( Nb,Cc,YZn),(Nb,Cc,YZo),(Nb,Cc,YZp),(Nb,Cc,YZq),(Nb,Cc,YZr),(Nb,Cc,YZs),(Nb,Cc,YZ t),(Nb,Cc,YZu),(Nb,Cc,YZv),(Nb,Cc,YZw),(Nb,Cc,YZx),(Nb,Cc,YZy),(Nb,Cc,YZz),(Nb,Cc ,YZaa),(Nb,Cc,YZab),(Nb,Cc,YZac),(Nb,Cc,YZad),(Nb,Cc,YZae),(Nb,Cc,YZaf),(Nb,Cc,YZ ag),(Nb,Cd,YZa),(Nb,Cd,YZb),(Nb,Cd,YZc),(Nb,Cd,YZd),(Nb,Cd,YZe),(Nb,Cd,YZf),(Nb,C d,YZg),(Nb,Cd,YZh),(Nb,Cd,YZi),(Nb,Cd,YZj),(Nb,Cd,YZk),(Nb,Cd,YZl),(Nb,Cd,YZm),( Nb,Cd,YZn),(Nb,Cd,YZo),(Nb,Cd,YZp),(Nb,Cd,YZq),(Nb,Cd,YZr),(Nb,Cd,YZs),(Nb,Cd,Y Zt),(Nb,Cd,YZu),(Nb,Cd,YZv),(Nb,Cd,YZw),(Nb,Cd,YZx),(Nb,Cd,YZy),(Nb,Cd,YZz),(Nb, Cd,YZaa),(Nb,Cd,YZab),(Nb,Cd,YZac),(Nb,Cd,YZad),(Nb,Cd,YZae),(Nb,Cd,YZaf),(Nb,Cd, YZag),(Nb,Ce,YZa),(Nb,Ce,YZb),(Nb,Ce,YZc),(Nb,Ce,YZd),(Nb,Ce,YZe),(Nb,Ce,YZf),(Nb ,Ce,YZg),(Nb,Ce,YZh),(Nb,Ce,YZi),(Nb,Ce,YZj),(Nb,Ce,YZk),(Nb,Ce,YZl),(Nb,Ce,YZm),( Nb,Ce,YZn),(Nb,Ce,YZo),(Nb,Ce,YZp),(Nb,Ce,YZq),(Nb,Ce,YZr),(Nb,Ce,YZs),(Nb,Ce,YZ t),(Nb,Ce,YZu),(Nb,Ce,YZv),(Nb,Ce,YZw),(Nb,Ce,YZx),(Nb,Ce,YZy),(Nb,Ce,YZz),(Nb,Ce ,YZaa),(Nb,Ce,YZab),(Nb,Ce,YZac),(Nb,Ce,YZad),(Nb,Ce,YZae),(Nb,Ce,YZaf),(Nb,Ce,YZ ag),(Nb,Cf,YZa),(Nb,Cf,YZb),(Nb,Cf,YZc),(Nb,Cf,YZd),(Nb,Cf,YZe),(Nb,Cf,YZf),(Nb,Cf, YZg),(Nb,Cf,YZh),(Nb,Cf,YZi),(Nb,Cf,YZj),(Nb,Cf,YZk),(Nb,Cf,YZl),(Nb,Cf,YZm),(Nb,C f,YZn),(Nb,Cf,YZo),(Nb,Cf,YZp),(Nb,Cf,YZq),(Nb,Cf,YZr),(Nb,Cf,YZs),(Nb,Cf,YZt),(Nb, Cf,YZu),(Nb,Cf,YZv),(Nb,Cf,YZw),(Nb,Cf,YZx),(Nb,Cf,YZy),(Nb,Cf,YZz),(Nb,Cf,YZaa),( Nb,Cf,YZab),(Nb,Cf,YZac),(Nb,Cf,YZad),(Nb,Cf,YZae),(Nb,Cf,YZaf),(Nb,Cf,YZag),(Nb,C g,YZa),(Nb,Cg,YZb),(Nb,Cg,YZc),(Nb,Cg,YZd),(Nb,Cg,YZe),(Nb,Cg,YZf),(Nb,Cg,YZg),( Nb,Cg,YZh),(Nb,Cg,YZi),(Nb,Cg,YZj),(Nb,Cg,YZk),(Nb,Cg,YZl),(Nb,Cg,YZm),(Nb,Cg,Y Zn),(Nb,Cg,YZo),(Nb,Cg,YZp),(Nb,Cg,YZq),(Nb,Cg,YZr),(Nb,Cg,YZs),(Nb,Cg,YZt),(Nb,C g,YZu),(Nb,Cg,YZv),(Nb,Cg,YZw),(Nb,Cg,YZx),(Nb,Cg,YZy),(Nb,Cg,YZz),(Nb,Cg,YZaa), (Nb,Cg,YZab),(Nb,Cg,YZac),(Nb,Cg,YZad),(Nb,Cg,YZae),(Nb,Cg,YZaf),(Nb,Cg,YZag),(N b,Ch,YZa),(Nb,Ch,YZb),(Nb,Ch,YZc),(Nb,Ch,YZd),(Nb,Ch,YZe),(Nb,Ch,YZf),(Nb,Ch,YZg) ,(Nb,Ch,YZh),(Nb,Ch,YZi),(Nb,Ch,YZj),(Nb,Ch,YZk),(Nb,Ch,YZl),(Nb,Ch,YZm),(Nb,Ch, YZn),(Nb,Ch,YZo),(Nb,Ch,YZp),(Nb,Ch,YZq),(Nb,Ch,YZr),(Nb,Ch,YZs),(Nb,Ch,YZt),(Nb, Ch,YZu),(Nb,Ch,YZv),(Nb,Ch,YZw),(Nb,Ch,YZx),(Nb,Ch,YZy),(Nb,Ch,YZz),(Nb,Ch,YZa a),(Nb,Ch,YZab),(Nb,Ch,YZac),(Nb,Ch,YZad),(Nb,Ch,YZae),(Nb,Ch,YZaf),(Nb,Ch,YZag),( Nb,Ci,YZa),(Nb,Ci,YZb),(Nb,Ci,YZc),(Nb,Ci,YZd),(Nb,Ci,YZe),(Nb,Ci,YZf),(Nb,Ci,YZg),( Nb,Ci,YZh),(Nb,Ci,YZi),(Nb,Ci,YZj),(Nb,Ci,YZk),(Nb,Ci,YZl),(Nb,Ci,YZm),(Nb,Ci,YZn),( Nb,Ci,YZo),(Nb,Ci,YZp),(Nb,Ci,YZq),(Nb,Ci,YZr),(Nb,Ci,YZs),(Nb,Ci,YZt),(Nb,Ci,YZu),( Nb,Ci,YZv),(Nb,Ci,YZw),(Nb,Ci,YZx),(Nb,Ci,YZy),(Nb,Ci,YZz),(Nb,Ci,YZaa),(Nb,Ci,YZa b),(Nb,Ci,YZac),(Nb,Ci,YZad),(Nb,Ci,YZae),(Nb,Ci,YZaf),(Nb,Ci,YZag),(Nb,Cj,YZa),(Nb, Cj,YZb),(Nb,Cj,YZc),(Nb,Cj,YZd),(Nb,Cj,YZe),(Nb,Cj,YZf),(Nb,Cj,YZg),(Nb,Cj,YZh),(Nb ,Cj,YZi),(Nb,Cj,YZj),(Nb,Cj,YZk),(Nb,Cj,YZl),(Nb,Cj,YZm),(Nb,Cj,YZn),(Nb,Cj,YZo),(Nb ,Cj,YZp),(Nb,Cj,YZq),(Nb,Cj,YZr),(Nb,Cj,YZs),(Nb,Cj,YZt),(Nb,Cj,YZu),(Nb,Cj,YZv),(Nb ,Cj,YZw),(Nb,Cj,YZx),(Nb,Cj,YZy),(Nb,Cj,YZz),(Nb,Cj,YZaa),(Nb,Cj,YZab),(Nb,Cj,YZac) ,(Nb,Cj,YZad),(Nb,Cj,YZae),(Nb,Cj,YZaf),(Nb,Cj,YZag),(Nc,Ca,YZa),(Nc,Ca,YZb),(Nc,Ca, YZc),(Nc,Ca,YZd),(Nc,Ca,YZe),(Nc,Ca,YZf),(Nc,Ca,YZg),(Nc,Ca,YZh),(Nc,Ca,YZi),(Nc,C a,YZj),(Nc,Ca,YZk),(Nc,Ca,YZl),(Nc,Ca,YZm),(Nc,Ca,YZn),(Nc,Ca,YZo),(Nc,Ca,YZp),(Nc ,Ca,YZq),(Nc,Ca,YZr),(Nc,Ca,YZs),(Nc,Ca,YZt),(Nc,Ca,YZu),(Nc,Ca,YZv),(Nc,Ca,YZw),( Nc,Ca,YZx),(Nc,Ca,YZy),(Nc,Ca,YZz),(Nc,Ca,YZaa),(Nc,Ca,YZab),(Nc,Ca,YZac),(Nc,Ca, YZad),(Nc,Ca,YZae),(Nc,Ca,YZaf),(Nc,Ca,YZag),(Nc,Cb,YZa),(Nc,Cb,YZb),(Nc,Cb,YZc),( Nc,Cb,YZd),(Nc,Cb,YZe),(Nc,Cb,YZf),(Nc,Cb,YZg),(Nc,Cb,YZh),(Nc,Cb,YZi),(Nc,Cb,YZj) ,(Nc,Cb,YZk),(Nc,Cb,YZl),(Nc,Cb,YZm),(Nc,Cb,YZn),(Nc,Cb,YZo),(Nc,Cb,YZp),(Nc,Cb, YZq),(Nc,Cb,YZr),(Nc,Cb,YZs),(Nc,Cb,YZt),(Nc,Cb,YZu),(Nc,Cb,YZv),(Nc,Cb,YZw),(Nc, Cb,YZx),(Nc,Cb,YZy),(Nc,Cb,YZz),(Nc,Cb,YZaa),(Nc,Cb,YZab),(Nc,Cb,YZac),(Nc,Cb,YZ ad),(Nc,Cb,YZae),(Nc,Cb,YZaf),(Nc,Cb,YZag),(Nc,Cc,YZa),(Nc,Cc,YZb),(Nc,Cc,YZc),(Nc, Cc,YZd),(Nc,Cc,YZe),(Nc,Cc,YZf),(Nc,Cc,YZg),(Nc,Cc,YZh),(Nc,Cc,YZi),(Nc,Cc,YZj),(N c,Cc,YZk),(Nc,Cc,YZl),(Nc,Cc,YZm),(Nc,Cc,YZn),(Nc,Cc,YZo),(Nc,Cc,YZp),(Nc,Cc,YZq) ,(Nc,Cc,YZr),(Nc,Cc,YZs),(Nc,Cc,YZt),(Nc,Cc,YZu),(Nc,Cc,YZv),(Nc,Cc,YZw),(Nc,Cc,YZ x),(Nc,Cc,YZy),(Nc,Cc,YZz),(Nc,Cc,YZaa),(Nc,Cc,YZab),(Nc,Cc,YZac),(Nc,Cc,YZad),(Nc, Cc,YZae),(Nc,Cc,YZaf),(Nc,Cc,YZag),(Nc,Cd,YZa),(Nc,Cd,YZb),(Nc,Cd,YZc),(Nc,Cd,YZd) ,(Nc,Cd,YZe),(Nc,Cd,YZf),(Nc,Cd,YZg),(Nc,Cd,YZh),(Nc,Cd,YZi),(Nc,Cd,YZj),(Nc,Cd,Y Zk),(Nc,Cd,YZl),(Nc,Cd,YZm),(Nc,Cd,YZn),(Nc,Cd,YZo),(Nc,Cd,YZp),(Nc,Cd,YZq),(Nc, Cd,YZr),(Nc,Cd,YZs),(Nc,Cd,YZt),(Nc,Cd,YZu),(Nc,Cd,YZv),(Nc,Cd,YZw),(Nc,Cd,YZx),( Nc,Cd,YZy),(Nc,Cd,YZz),(Nc,Cd,YZaa),(Nc,Cd,YZab),(Nc,Cd,YZac),(Nc,Cd,YZad),(Nc,C d,YZae),(Nc,Cd,YZaf),(Nc,Cd,YZag),(Nc,Ce,YZa),(Nc,Ce,YZb),(Nc,Ce,YZc),(Nc,Ce,YZd), (Nc,Ce,YZe),(Nc,Ce,YZf),(Nc,Ce,YZg),(Nc,Ce,YZh),(Nc,Ce,YZi),(Nc,Ce,YZj),(Nc,Ce,YZk) ,(Nc,Ce,YZl),(Nc,Ce,YZm),(Nc,Ce,YZn),(Nc,Ce,YZo),(Nc,Ce,YZp),(Nc,Ce,YZq),(Nc,Ce, YZr),(Nc,Ce,YZs),(Nc,Ce,YZt),(Nc,Ce,YZu),(Nc,Ce,YZv),(Nc,Ce,YZw),(Nc,Ce,YZx),(Nc, Ce,YZy),(Nc,Ce,YZz),(Nc,Ce,YZaa),(Nc,Ce,YZab),(Nc,Ce,YZac),(Nc,Ce,YZad),(Nc,Ce,YZ ae),(Nc,Ce,YZaf),(Nc,Ce,YZag),(Nc,Cf,YZa),(Nc,Cf,YZb),(Nc,Cf,YZc),(Nc,Cf,YZd),(Nc,Cf ,YZe),(Nc,Cf,YZf),(Nc,Cf,YZg),(Nc,Cf,YZh),(Nc,Cf,YZi),(Nc,Cf,YZj),(Nc,Cf,YZk),(Nc,Cf, YZl),(Nc,Cf,YZm),(Nc,Cf,YZn),(Nc,Cf,YZo),(Nc,Cf,YZp),(Nc,Cf,YZq),(Nc,Cf,YZr),(Nc,Cf ,YZs),(Nc,Cf,YZt),(Nc,Cf,YZu),(Nc,Cf,YZv),(Nc,Cf,YZw),(Nc,Cf,YZx),(Nc,Cf,YZy),(Nc,C f,YZz),(Nc,Cf,YZaa),(Nc,Cf,YZab),(Nc,Cf,YZac),(Nc,Cf,YZad),(Nc,Cf,YZae),(Nc,Cf,YZaf) ,(Nc,Cf,YZag),(Nc,Cg,YZa),(Nc,Cg,YZb),(Nc,Cg,YZc),(Nc,Cg,YZd),(Nc,Cg,YZe),(Nc,Cg, YZf),(Nc,Cg,YZg),(Nc,Cg,YZh),(Nc,Cg,YZi),(Nc,Cg,YZj),(Nc,Cg,YZk),(Nc,Cg,YZl),(Nc,C g,YZm),(Nc,Cg,YZn),(Nc,Cg,YZo),(Nc,Cg,YZp),(Nc,Cg,YZq),(Nc,Cg,YZr),(Nc,Cg,YZs),( Nc,Cg,YZt),(Nc,Cg,YZu),(Nc,Cg,YZv),(Nc,Cg,YZw),(Nc,Cg,YZx),(Nc,Cg,YZy),(Nc,Cg,Y Zz),(Nc,Cg,YZaa),(Nc,Cg,YZab),(Nc,Cg,YZac),(Nc,Cg,YZad),(Nc,Cg,YZae),(Nc,Cg,YZaf), (Nc,Cg,YZag),(Nc,Ch,YZa),(Nc,Ch,YZb),(Nc,Ch,YZc),(Nc,Ch,YZd),(Nc,Ch,YZe),(Nc,Ch, Ya),(Nc,Ch,YZg),(Nc,Ch,YZh),(Nc,Ch,YZi),(Nc,Ch,YZj),(Nc,Ch,YZk),(Nc,Ch,YZl),(Nc,C h,YZm),(Nc,Ch,YZn),(Nc,Ch,YZo),(Nc,Ch,YZp),(Nc,Ch,YZq),(Nc,Ch,YZr),(Nc,Ch,YZs),( Nc,Ch,YZt),(Nc,Ch,YZu),(Nc,Ch,YZv),(Nc,Ch,YZw),(Nc,Ch,YZx),(Nc,Ch,YZy),(Nc,Ch,Y Zz),(Nc,Ch,YZaa),(Nc,Ch,YZab),(Nc,Ch,YZac),(Nc,Ch,YZad),(Nc,Ch,YZae),(Nc,Ch,YZaf), (Nc,Ch,YZag),(Nc,Ci,Yza),(Nc,Ci,YZb),(Nc,Ci,YZc),(Nc,Ci,YZd),(Nc,Ci,YZe),(Nc,Ci,YZf) ,(Nc,Ci,YZg),(Nc,Ci,YZh),(Nc,Ci,YZi),(Nc,Ci,YZj),(Nc,Ci,YZk),(Nc,Ci,YZl),(Nc,Ci,YZm), (Nc,Ci,YZn),(Nc,Ci,YZo),(Nc,Ci,YZp),(Nc,Ci,YZq),(Nc,Ci,YZr),(Nc,Ci,YZs),(Nc,Ci,YZt),( Nc,Ci,YZu),(Nc,Ci,YZv),(Nc,Ci,YZw),(Nc,Ci,YZx),(Nc,Ci,YZy),(Nc,Ci,YZz),(Nc,Ci,YZaa) ,(Nc,Ci,YZab),(Nc,Ci,YZac),(Nc,Ci,YZad),(Nc,Ci,YZae),(Nc,Ci,YZaf),(Nc,Ci,YZag),(Nc,Cj ,Yza),(Nc,Cj,YZb),(Nc,Cj,YZc),(Nc,Cj,YZd),(Nc,Cj,YZe),(Nc,Cj,YZf),(Nc,Cj,YZg),(Nc,Cj, YZh),(Nc,Cj,YZi),(Nc,Cj,YZj),(Nc,Cj,YZk),(Nc,Cj,YZl),(Nc,Cj,YZm),(Nc,Cj,YZn),(Nc,Cj, YZo),(Nc,Cj,YZp),(Nc,Cj,YZq),(Nc,Cj,YZr),(Nc,Cj,YZs),(Nc,Cj,YZt),(Nc,Cj,YZu),(Nc,Cj, YZv),(Nc,Cj,YZw),(Nc,Cj,YZx),(Nc,Cj,YZy),(Nc,Cj,YZz),(Nc,Cj,YZaa),(Nc,Cj,YZab),(Nc, Cj,YZac),(Nc,Cj,YZad),(Nc,Cj,YZae),(Nc,Cj,YZaf),(Nc,Cj,YZag),(Nd,Ca,YZa),(Nd,Ca,YZb) ,(Nd,Ca,YZc),(Nd,Ca,YZd),(Nd,Ca,YZe),(Nd,Ca,YZf),(Nd,Ca,YZg),(Nd,Ca,YZh),(Nd,Ca, YZi),(Nd,Ca,YZj),(Nd,Ca,YZk),(Nd,Ca,YZl),(Nd,Ca,YZm),(Nd,Ca,YZn),(Nd,Ca,YZo),(Nd, Ca,YZp),(Nd,Ca,YZq),(Nd,Ca,YZr),(Nd,Ca,YZs),(Nd,Ca,YZt),(Nd,Ca,YZu),(Nd,Ca,YZv),( Nd,Ca,YZw),(Nd,Ca,YZx),(Nd,Ca,YZy),(Nd,Ca,YZz),(Nd,Ca,YZaa),(Nd,Ca,YZab),(Nd,Ca, YZac),(Nd,Ca,YZad),(Nd,Ca,YZae),(Nd,Ca,YZaf),(Nd,Ca,YZag),(Nd,Cb,YZa),(Nd,Cb,YZb) ,(Nd,Cb,YZc),(Nd,Cb,YZd),(Nd,Cb,YZe),(Nd,Cb,YZf),(Nd,Cb,YZg),(Nd,Cb,YZh),(Nd,Cb, YZi),(Nd,Cb,YZj),(Nd,Cb,YZk),(Nd,Cb,YZl),(Nd,Cb,YZm),(Nd,Cb,YZn),(Nd,Cb,YZo),(Nd ,Cb,YZp),(Nd,Cb,YZq),(Nd,Cb,YZr),(Nd,Cb,YZs),(Nd,Cb,YZt),(Nd,Cb,YZu),(Nd,Cb,YZv), (Nd,Cb,YZw),(Nd,Cb,YZx),(Nd,Cb,YZy),(Nd,Cb,YZz),(Nd,Cb,YZaa),(Nd,Cb,YZab),(Nd,C b,YZac),(Nd,Cb,YZad),(Nd,Cb,YZae),(Nd,Cb,YZaf),(Nd,Cb,YZag),(Nd,Cc,YZa),(Nd,Cc,Y Zb),(Nd,Cc,YZc),(Nd,Cc,YZd),(Nd,Cc,YZe),(Nd,Cc,YZf),(Nd,Cc,YZg),(Nd,Cc,YZh),(Nd,C c,YZi),(Nd,Cc,YZj),(Nd,Cc,YZk),(Nd,Cc,YZl),(Nd,Cc,YZm),(Nd,Cc,YZn),(Nd,Cc,YZo),(N d,Cc,YZp),(Nd,Cc,YZq),(Nd,Cc,YZr),(Nd,Cc,YZs),(Nd,Cc,YZt),(Nd,Cc,YZu),(Nd,Cc,YZv), (Nd,Cc,YZw),(Nd,Cc,YZx),(Nd,Cc,YZy),(Nd,Cc,YZz),(Nd,Cc,YZaa),(Nd,Cc,YZab),(Nd,Cc ,YZac),(Nd,Cc,YZad),(Nd,Cc,YZae),(Nd,Cc,YZaf),(Nd,Cc,YZag),(Nd,Cd,YZa),(Nd,Cd,YZb) ,(Nd,Cd,YZc),(Nd,Cd,YZd),(Nd,Cd,YZe),(Nd,Cd,YZf),(Nd,Cd,YZg),(Nd,Cd,YZh),(Nd,Cd, YZi),(Nd,Cd,YZj),(Nd,Cd,YZk),(Nd,Cd,YZl),(Nd,Cd,YZm),(Nd,Cd,YZn),(Nd,Cd,YZo),(Nd ,Cd,YZp),(Nd,Cd,YZq),(Nd,Cd,YZr),(Nd,Cd,YZs),(Nd,Cd,YZt),(Nd,Cd,YZu),(Nd,Cd,YZv), (Nd,Cd,YZw),(Nd,Cd,YZx),(Nd,Cd,YZy),(Nd,Cd,YZz),(Nd,Cd,YZaa),(Nd,Cd,YZab),(Nd,C d,YZac),(Nd,Cd,YZad),(Nd,Cd,YZae),(Nd,Cd,YZaf),(Nd,Cd,YZag),(Nd,Ce,YZa),(Nd,Ce,Y Zb),(Nd,Ce,YZc),(Nd,Ce,YZd),(Nd,Ce,YZe),(Nd,Ce,YZf),(Nd,Ce,YZg),(Nd,Ce,YZh),(Nd,C e,YZi),(Nd,Ce,YZj),(Nd,Ce,YZl),(Nd,Ce,YZl),(Nd,Ce,YZm),(Nd,Ce,YZn),(Nd,Ce,YZo),(N d,Ce,YZp),(Nd,Ce,YZq),(Nd,Ce,YZr),(Nd,Ce,YZs),(Nd,Ce,YZt),(Nd,Ce,YZu),(Nd,Ce,YZv), (Nd,Ce,YZw),(Nd,Ce,YZx),(Nd,Ce,YZy),(Nd,Ce,YZz),(Nd,Ce,YZaa),(Nd,Ce,YZab),(Nd,Ce ,YZac),(Nd,Ce,YZad),(Nd,Ce,YZae),(Nd,Ce,YZaf),(Nd,Ce,YZag),(Nd,Cf,YZa),(Nd,Cf,YZb) ,(Nd,Cf,YZc),(Nd,Cf,YZd),(Nd,Cf,YZe),(Nd,Cf,YZf),(Nd,Cf,YZg),(Nd,Cf,Yh),(Nd,Cf,YZi) ,(Nd,Cf,YZj),(Nd,Cf,YZk),(Nd,Cf,YZl),(Nd,Cf,YZm),(Nd,Cf,YZn),(Nd,Cf,YZo),(Nd,Cf,Y Zp),(Nd,Cf,YZq),(Nd,Cf,YZr),(Nd,Cf,YZs),(Nd,Cf,YZt),(Nd,Cf,YZu),(Nd,Cf,YZv),(Nd,Cf, YZw),(Nd,Cf,YZx),(Nd,Cf,YZy),(Nd,Cf,YZz),(Nd,Cf,YZaa),(Nd,Cf,YZab),(Nd,Cf,YZac),( Nd,Cf,YZad),(Nd,Cf,YZae),(Nd,Cf,YZaf),(Nd,Cf,YZag),(Nd,Cg,YZa),(Nd,Cg,YZb),(Nd,Cg, YZc),(Nd,Cg,YZd),(Nd,Cg,YZe),(Nd,Cg,YZf),(Nd,Cg,YZg),(Nd,Cg,YZh),(Nd,Cg,YZi),(Nd, Cg,YZj),(Nd,Cg,YZk),(Nd,Cg,YZl),(Nd,Cg,YZm),(Nd,Cg,YZn),(Nd,Cg,YZo),(Nd,Cg,YZp), (Nd,Cg,YZq),(Nd,Cg,YZr),(Nd,Cg,YZs),(Nd,Cg,YZt),(Nd,Cg,YZu),(Nd,Cg,YZv),(Nd,Cg,Y Zw),(Nd,Cg,YZx),(Nd,Cg,YZy),(Nd,Cg,YZz),(Nd,Cg,YZaa),(Nd,Cg,YZab),(Nd,Cg,YZac),( Nd,Cg,YZad),(Nd,Cg,YZae),(Nd,Cg,YZaf),(Nd,Cg,YZag),(Nd,Ch,YZa),(Nd,Ch,YZb),(Nd,C h,YZc),(Nd,Ch,YZd),(Nd,Ch,YZe),(Nd,Ch,YZf),(Nd,Ch,YZg),(Nd,Ch,YZh),(Nd,Ch,YZi),(N d,Ch,YZj),(Nd,Ch,YZk),(Nd,Ch,YZl),(Nd,Ch,YZm),(Nd,Ch,YZn),(Nd,Ch,YZo),(Nd,Ch,YZ p),(Nd,Ch,YZq),(Nd,Ch,YZr),(Nd,Ch,YZs),(Nd,Ch,YZt),(Nd,Ch,YZu),(Nd,Ch,YZv),(Nd,Ch ,YZw),(Nd,Ch,YZx),(Nd,Ch,YZy),(Nd,Ch,YZz),(Nd,Ch,YZaa),(Nd,Ch,YZab),(Nd,Ch,YZac) ,(Nd,Ch,YZad),(Nd,Ch,YZae),(Nd,Ch,YZaf),(Nd,Ch,YZag),(Nd,Ci,YZa),(Nd,Ci,YZb),(Nd, Ci,YZc),(Nd,Ci,YZd),(Nd,Ci,YZe),(Nd,Ci,YZf),(Nd,Ci,YZg),(Nd,Ci,YZh),(Nd,Ci,YZi),(Nd, Ci,YZj),(Nd,Ci,YZk),(Nd,Ci,YZl),(Nd,Ci,YZm),(Nd,Ci,YZn),(Nd,Ci,YZo),(Nd,Ci,YZp),(Nd ,Ci,YZq),(Nd,Ci,YZr),(Nd,Ci,YZs),(Nd,Ci,YZt),(Nd,Ci,YZu),(Nd,Ci,YZv),(Nd,Ci,YZw),(N d,Ci,YZx),(Nd,Ci,YZy),(Nd,Ci,YZz),(Nd,Ci,YZaa),(Nd,Ci,YZab),(Nd,Ci,YZac),(Nd,Ci,YZa d),(Nd,Ci,YZae),(Nd,Ci,YZaf),(Nd,Ci,YZag),(Nd,Cj,YZa),(Nd,Cj,YZb),(Nd,Cj,YZc),(Nd,Cj, YZd),(Nd,Cj,YZe),(Nd,Cj,YZf),(Nd,Cj,YZg),(Nd,Cj,YZh),(Nd,Cj,YZi),(Nd,Cj,YZj),(Nd,Cj, YZk),(Nd,Cj,YZl),(Nd,Cj,YZm),(Nd,Cj,YZn),(Nd,Cj,YZo),(Nd,Cj,YZp),(Nd,Cj,YZq),(Nd,C j,YZr),(Nd,Cj,YZs),(Nd,Cj,YZt),(Nd,Cj,YZu),(Nd,Cj,YZv),(Nd,Cj,YZw),(Nd,Cj,YZx),(Nd, Cj,YZy),(Nd,Cj,YZz),(Nd,Cj,YZaa),(Nd,Cj,YZab),(Nd,Cj,YZac),(Nd,Cj,YZad),(Nd,Cj,YZae) ,(Nd,Cj,YZaf),(Nd,Cj,YZag),(Ne,Ca,YZa),(Ne,Ca,YZb),(Ne,Ca,YZc),(Ne,Ca,YZd),(Ne,Ca, YZe),(Ne,Ca,YZf),(Ne,Ca,YZg),(Ne,Ca,YZh),(Ne,Ca,YZi),(Ne,Ca,YZj),(Ne,Ca,YZk),(Ne,C a,YZl),(Ne,Ca,YZm),(Ne,Ca,YZn),(Ne,Ca,YZo),(Ne,Ca,YZp),(Ne,Ca,YZq),(Ne,Ca,YZr),(N e,Ca,YZs),(Ne,Ca,YZt),(Ne,Ca,YZu),(Ne,Ca,YZv),(Ne,Ca,YZw),(Ne,Ca,YZx),(Ne,Ca,YZy), (Ne,Ca,YZz),(Ne,Ca,YZaa),(Ne,Ca,YZab),(Ne,Ca,YZac),(Ne,Ca,YZad),(Ne,Ca,YZae),(Ne,C a,YZaf),(Ne,Ca,YZag),(Ne,Cb,YZa),(Ne,Cb,YZb),(Ne,Cb,YZc),(Ne,Cb,YZd),(Ne,Cb,YZe),( Ne,Cb,YZf),(Ne,Cb,YZg),(Ne,Cb,YZh),(Ne,Cb,YZi),(Ne,Cb,YZj),(Ne,Cb,YZk),(Ne,Cb,YZl) ,(Ne,Cb,YZm),(Ne,Cb,YZn),(Ne,Cb,YZo),(Ne,Cb,YZp),(Ne,Cb,YZq),(Ne,Cb,YZr),(Ne,Cb, YZs),(Ne,Cb,YZt),(Ne,Cb,YZu),(Ne,Cb,YZv),(Ne,Cb,YZw),(Ne,Cb,YZx),(Ne,Cb,YZy),(Ne, Cb,YZz),(Ne,Cb,YZaa),(Ne,Cb,YZab),(Ne,Cb,YZac),(Ne,Cb,YZad),(Ne,Cb,YZae),(Ne,Cb, YZaf),(Ne,Cb,YZag),(Ne,Cc,YZa),(Ne,Cc,YZb),(Ne,Cc,YZc),(Ne,Cc,YZd),(Ne,Cc,YZe),(N e,Cc,YZf),(Ne,Cc,YZg),(Ne,Cc,YZh),(Ne,Cc,YZi),(Ne,Cc,YZj),(Ne,Cc,YZk),(Ne,Cc,YZl),( Ne,Cc,YZm),(Ne,Cc,YZn),(Ne,Cc,YZo),(Ne,Cc,YZp),(Ne,Cc,YZq),(Ne,Cc,YZr),(Ne,Cc,YZ s),(Ne,Cc,YZt),(Ne,Cc,YZu),(Ne,Cc,YZv),(Ne,Cc,YZw),(Ne,Cc,YZx),(Ne,Cc,YZy),(Ne,Cc, YZz),(Ne,Cc,YZaa),(Ne,Cc,YZab),(Ne,Cc,YZac),(Ne,Cc,YZad),(Ne,Cc,YZae),(Ne,Cc,YZaf) ,(Ne,Cc,YZag),(Ne,Cd,YZa),(Ne,Cd,YZb),(Ne,Cd,YZc),(Ne,Cd,YZd),(Ne,Cd,YZe),(Ne,Cd, YZf),(Ne,Cd,YZg),(Ne,Cd,YZh),(Ne,Cd,YZi),(Ne,Cd,YZj),(Ne,Cd,YZk),(Ne,Cd,YZl),(Ne,C d,YZm),(Ne,Cd,YZn),(Ne,Cd,YZo),(Ne,Cd,YZp),(Ne,Cd,YZq),(Ne,Cd,YZr),(Ne,Cd,YZs),( Ne,Cd,YZt),(Ne,Cd,YZu),(Ne,Cd,YZv),(Ne,Cd,YZw),(Ne,Cd,YZx),(Ne,Cd,YZy),(Ne,Cd,Y Zz),(Ne,Cd,YZaa),(Ne,Cd,YZab),(Ne,Cd,YZac),(Ne,Cd,YZad),(Ne,Cd,YZae),(Ne,Cd,YZaf), (Ne,Cd,YZag),(Ne,Ce,YZa),(Ne,Ce,YZb),(Ne,Ce,YZc),(Ne,Ce,YZd),(Ne,Ce,YZe),(Ne,Ce,Y Zf),(Ne,Ce,YZg),(Ne,Ce,YZh),(Ne,Ce,YZi),(Ne,Ce,YZj),(Ne,Ce,YZk),(Ne,Ce,YZl),(Ne,Ce, YZm),(Ne,Ce,YZn),(Ne,Ce,YZo),(Ne,Ce,YZp),(Ne,Ce,YZq),(Ne,Ce,YZr),(Ne,Ce,YZs),(Ne, Ce,YZt),(Ne,Ce,YZu),(Ne,Ce,YZv),(Ne,Ce,YZw),(Ne,Ce,YZx),(Ne,Ce,YZy),(Ne,Ce,YZz),( Ne,Ce,YZaa),(Ne,Ce,YZab),(Ne,Ce,YZac),(Ne,Ce,YZad),(Ne,Ce,YZae),(Ne,Ce,YZaf),(Ne,C e,YZag),(Ne,Cf,YZa),(Ne,Cf,YZb),(Ne,Cf,YZc),(Ne,Cf,YZd),(Ne,Cf,YZe),(Ne,Cf,YZf),(Ne, Cf,YZg),(Ne,Cf,YZh),(Ne,Cf,YZi),(Ne,Cf,YZj),(Ne,Cf,YZk),(Ne,Cf,YZl),(Ne,Cf,YZm),(Ne, Cf,YZn),(Ne,Cf,YZo),(Ne,Cf,YZp),(Ne,Cf,YZq),(Ne,Cf,YZr),(Ne,Cf,YZs),(Ne,Cf,YZt),(Ne, Cf,YZu),(Ne,Cf,YZv),(Ne,Cf,YZw),(Ne,Cf,YZx),(Ne,Cf,YZy),(Ne,Cf,YZz),(Ne,Cf,YZaa),( Ne,Cf,YZab),(Ne,Cf,YZac),(Ne,Cf,YZad),(Ne,Cf,YZae),(Ne,Cf,YZaf),(Ne,Cf,YZag),(Ne,Cg ,YZa),(Ne,Cg,YZb),(Ne,Cg,YZc),(Ne,Cg,YZd),(Ne,Cg,YZe),(Ne,Cg,YZf),(Ne,Cg,YZg),(Ne, Cg,YZh),(Ne,Cg,YZi),(Ne,Cg,YZj),(Ne,Cg,YZk),(Ne,Cg,YZl),(Ne,Cg,YZm),(Ne,Cg,YZn),( Ne,Cg,YZo),(Ne,Cg,YZp),(Ne,Cg,YZq),(Ne,Cg,YZr),(Ne,Cg,YZs),(Ne,Cg,YZt),(Ne,Cg,YZ u),(Ne,Cg,YZv),(Ne,Cg,YZw),(Ne,Cg,YZx),(Ne,Cg,YZy),(Ne,Cg,YZz),(Ne,Cg,YZaa),(Ne,C g,YZab),(Ne,Cg,YZac),(Ne,Cg,YZad),(Ne,Cg,YZae),(Ne,Cg,YZaf),(Ne,Cg,YZag),(Ne,Ch,Y Za),(Ne,Ch,YZb),(Ne,Ch,YZc),(Ne,Ch,YZd),(Ne,Ch,YZe),(Ne,Ch,YZf),(Ne,Ch,YZg),(Ne,C h,YZh),(Ne,Ch,YZi),(Ne,Ch,YZj),(Ne,Ch,YZk),(Ne,Ch,YZl),(Ne,Ch,YZm),(Ne,Ch,YZn),(N e,Ch,YZo),(Ne,Ch,YZp),(Ne,Ch,YZq),(Ne,Ch,YZr),(Ne,Ch,YZs),(Ne,Ch,YZt),(Ne,Ch,YZu), (Ne,Ch,YZv),(Ne,Ch,YZw),(Ne,Ch,YZx),(Ne,Ch,YZy),(Ne,Ch,YZz),(Ne,Ch,YZaa),(Ne,Ch, YZab),(Ne,Ch,YZac),(Ne,Ch,YZad),(Ne,Ch,YZae),(Ne,Ch,YZaf),(Ne,Ch,YZag),(Ne,Ci,YZa) ,(Ne,Ci,YZb),(Ne,Ci,YZc),(Ne,Ci,YZd),(Ne,Ci,YZe),(Ne,Ci,YZf),(Ne,Ci,YZg),(Ne,Ci,YZh) ,(Ne,Ci,YZi),(Ne,Ci,YZj),(Ne,Ci,YZk),(Ne,Ci,YZl),(Ne,Ci,YZm),(Ne,Ci,YZn),(Ne,Ci,YZo), (Ne,Ci,YZp),(Ne,Ci,YZq),(Ne,Ci,YZr),(Ne,Ci,YZs),(Ne,Ci,YZt),(Ne,Ci,YZu),(Ne,Ci,YZv),( Ne,Ci,YZw),(Ne,Ci,YZx),(Ne,Ci,YZy),(Ne,Ci,YZz),(Ne,Ci,YZaa),(Ne,Ci,YZab),(Ne,Ci,YZa c),(Ne,Ci,YZad),(Ne,Ci,YZae),(Ne,Ci,YZaf),(Ne,Ci,YZag),(Ne,Cj,YZa),(Ne,Cj,YZb),(Ne,Cj ,YZc),(Ne,Cj,YZd),(Ne,Cj,YZe),(Ne,Cj,YZf),(Ne,Cj,YZg),(Ne,Cj,YZh),(Ne,Cj,YZi),(Ne,Cj, YZj),(Ne,Cj,Ya),(Ne,Cj,YZl),(Ne,Cj,YZm),(Ne,Cj,YZn),(Ne,Cj,YZo),(Ne,Cj,YZp),(Ne,Cj, YZq),(Ne,Cj,YZr),(Ne,Cj,YZs),(Ne,Cj,YZt),(Ne,Cj,YZu),(Ne,Cj,YZv),(Ne,Cj,YZw),(Ne,Cj, YZx),(Ne,Cj,YZy),(Ne,Cj,YZz),(Ne,Cj,YZaa),(Ne,Cj,YZab),(Ne,Cj,YZac),(Ne,Cj,YZad),(N e,Cj,YZae),(Ne,Cj,YZaf),(Ne,Cj,YZag),(Nf,Ca,YZa),(Nf,Ca,YZb),(Nf,Ca,YZc),(Nf,Ca,YZd) ,(Nf,Ca,YZe),(Nf,Ca,YZf),(Nf,Ca,YZg),(Nf,Ca,YZh),(Nf,Ca,YZi),(Nf,Ca,YZj),(Nf,Ca,YZk), (Nf,Ca,YZl),(Nf,Ca,YZm),(Nf,Ca,YZn),(Nf,Ca,YZo),(Nf,Ca,YZp),(Nf,Ca,YZq),(Nf,Ca,YZr) ,(Nf,Ca,YZs),(Nf,Ca,YZt),(Nf,Ca,YZu),(Nf,Ca,YZv),(Nf,Ca,YZw),(Nf,Ca,YZx),(Nf,Ca,YZy) ,(Nf,Ca,YZz),(Nf,Ca,YZaa),(Nf,Ca,YZab),(Nf,Ca,YZac),(Nf,Ca,YZad),(Nf,Ca,YZae),(Nf,C a,YZaf),(Nf,Ca,YZag),(Nf,Cb,YZa),(Nf,Cb,YZb),(Nf,Cb,YZc),(Nf,Cb,YZd),(Nf,Cb,YZe),(N f,Cb,YZf),(Nf,Cb,YZg),(Nf,Cb,YZh),(Nf,Cb,YZi),(Nf,Cb,YZj),(Nf,Cb,YZk),(Nf,Cb,YZl),(N f,Cb,YZm),(Nf,Cb,YZn),(Nf,Cb,YZo),(Nf,Cb,YZp),(Nf,Cb,YZq),(Nf,Cb,YZr),(Nf,Cb,YZs),( Nf,Cb,YZt),(Nf,Cb,YZu),(Nf,Cb,YZv),(Nf,Cb,YZw),(Nf,Cb,YZx),(Nf,Cb,YZy),(Nf,Cb,YZz) ,(Nf,Cb,YZaa),(Nf,Cb,YZab),(Nf,Cb,YZac),(Nf,Cb,YZad),(Nf,Cb,YZae),(Nf,Cb,YZaf),(Nf, Cb,YZag),(Nf,Cc,YZa),(Nf,Cc,YZb),(Nf,Cc,YZc),(Nf,Cc,YZd),(Nf,Cc,YZe),(Nf,Cc,YZf),(N f,Cc,YZg),(Nf,Cc,YZh),(Nf,Cc,YZi),(Nf,Cc,YZj),(Nf,Cc,YZk),(Nf,Cc,YZl),(Nf,Cc,YZm),(N f,Cc,YZn),(Nf,Cc,YZo),(Nf,Cc,YZp),(Nf,Cc,YZq),(Nf,Cc,YZr),(Nf,Cc,YZs),(Nf,Cc,YZt),(N f,Cc,YZu),(Nf,Cc,YZv),(Nf,Cc,YZw),(Nf,Cc,YZx),(Nf,Cc,YZy),(Nf,Cc,YZz),(Nf,Cc,YZaa), (Nf,Cc,YZab),(Nf,Cc,YZac),(Nf,Cc,YZad),(Nf,Cc,YZae),(Nf,Cc,YZaf),(Nf,Cc,YZag),(Nf,C d,YZa),(Nf,Cd,YZb),(Nf,Cd,YZc),(Nf,Cd,YZd),(Nf,Cd,YZe),(Nf,Cd,YZf),(Nf,Cd,YZg),(Nf, Cd,YZh),(Nf,Cd,YZi),(Nf,Cd,YZj),(Nf,Cd,YZk),(Nf,Cd,YZl),(Nf,Cd,YZm),(Nf,Cd,YZn),(N f,Cd,YZo),(Nf,Cd,YZp),(Nf,Cd,YZq),(Nf,Cd,YZr),(Nf,Cd,YZs),(Nf,Cd,YZt),(Nf,Cd,YZu),( Nf,Cd,YZv),(Nf,Cd,YZw),(Nf,Cd,YZx),(Nf,Cd,YZy),(Nf,Cd,YZz),(Nf,Cd,YZaa),(Nf,Cd,YZ ab),(Nf,Cd,YZac),(Nf,Cd,YZad),(Nf,Cd,YZae),(Nf,Cd,YZaf),(Nf,Cd,YZag),(Nf,Ce,YZa),(Nf ,Ce,YZb),(Nf,Ce,YZc),(Nf,Ce,YZd),(Nf,Ce,YZe),(Nf,Ce,YZf),(Nf,Ce,YZg),(Nf,Ce,YZh),(N f,Ce,YZi),(Nf,Ce,YZj),(Nf,Ce,YZk),(Nf,Ce,YZl),(Nf,Ce,YZm),(Nf,Ce,YZn),(Nf,Ce,YZo),(N f,Ce,YZp),(Nf,Ce,YZq),(Nf,Ce,YZr),(Nf,Ce,YZs),(Nf,Ce,YZt),(Nf,Ce,YZu),(Nf,Ce,YZv),(N f,Ce,YZw),(Nf,Ce,YZx),(Nf,Ce,YZy),(Nf,Ce,YZz),(Nf,Ce,YZaa),(Nf,Ce,YZab),(Nf,Ce,YZa c),(Nf,Ce,YZad),(Nf,Ce,YZae),(Nf,Ce,YZaf),(Nf,Ce,YZag),(Nf,Cf,YZa),(Nf,Cf,YZb),(Nf,Cf ,YZc),(Nf,Cf,YZd),(Nf,Cf,YZe),(Nf,Cf,YZf),(Nf,Cf,YZg),(Nf,Cf,YZh),(Nf,Cf,YZi),(Nf,Cf,Y Zj),(Nf,Cf,YZk),(Nf,Cf,YZl),(Nf,Cf,YZm),(Nf,Cf,YZn),(Nf,Cf,YZo),(Nf,Cf,YZp),(Nf,Cf,YZ q),(Nf,Cf,YZr),(Nf,Cf,YZs),(Nf,Cf,YZt),(Nf,Cf,YZu),(Nf,Cf,YZv),(Nf,Cf,YZw),(Nf,Cf,YZx) ,(Nf,Cf,YZy),(Nf,Cf,YZz),(Nf,Cf,YZaa),(Nf,Cf,YZab),(Nf,Cf,YZac),(Nf,Cf,YZad),(Nf,Cf, YZae),(Nf,Cf,YZaf),(Nf,Cf,YZag),(Nf,Cg,YZa),(Nf,Cg,YZb),(Nf,Cg,YZc),(Nf,Cg,YZd),(Nf, Cg,YZe),(Nf,Cg,YZf),(Nf,Cg,YZg),(Nf,Cg,YZl),(Nf,Cg,YZi),(Nf,Cg,YZj),(Nf,Cg,YZk),(Nf ,Cg,YZl),(Nf,Cg,YZm),(Nf,Cg,YZn),(Nf,Cg,YZo),(Nf,Cg,YZp),(Nf,Cg,YZq),(Nf,Cg,YZr),( Nf,Cg,YZs),(Nf,Cg,YZt),(Nf,Cg,YZu),(Nf,Cg,YZv),(Nf,Cg,YZw),(Nf,Cg,YZx),(Nf,Cg,YZy) ,(Nf,Cg,YZz),(Nf,Cg,YZaa),(Nf,Cg,YZab),(Nf,Cg,YZac),(Nf,Cg,YZad),(Nf,Cg,YZae),(Nf,C g,YZaf),(Nf,Cg,YZag),(Nf,Ch,YZa),(Nf,Ch,YZb),(Nf,Ch,YZc),(Nf,Ch,YZd),(Nf,Ch,YZe),(N f,Ch,YZf),(Nf,Ch,YZg),(Nf,Ch,YZh),(Nf,Ch,YZi),(Nf,Ch,YZj),(Nf,Ch,YZk),(Nf,Ch,YZl),(N f,Ch,YZm),(Nf,Ch,YZn),(Nf,Ch,YZo),(Nf,Ch,YZp),(Nf,Ch,YZq),(Nf,Ch,YZr),(Nf,Ch,YZs),( Nf,Ch,YZt),(Nf,Ch,YZu),(Nf,Ch,YZv),(Nf,Ch,YZw),(Nf,Ch,YZx),(Nf,Ch,YZy),(Nf,Ch,YZz) ,(Nf,Ch,YZaa),(Nf,Ch,YZab),(NtCh,YZac),(Nf,Ch,YZad),(Nf,Ch,YZae),(Nf,Ch,YZaf),(Nf, Ch,YZag),(Nf,Ci,YZa),(Nf,Ci,YZb),(Nf,Ci,YZc),(Nf,Ci,YZd),(Nf,Ci,YZe),(Nf,Ci,YZf),(Nf, Ci,YZg),(Nf,Ci,YZh),(Nf,Ci,YZi),(Nf,Ci,YZj),(Nf,Ci,YZk),(Nf,Ci,YZl),(Nf,Ci,YZm),(Nf,Ci, YZn),(Nf,Ci,YZo),(Nf,Ci,YZp),(Nf,Ci,YZq),(Nf,Ci,YZr),(Nf,Ci,YZs),(Nf,Ci,YZt),(Nf,Ci,YZ u),(Nf,Ci,YZv),(Nf,Ci,YZw),(Nf,Ci,YZx),(Nf,Ci,YZy),(Nf,Ci,YZz),(Nf,Ci,YZaa),(Nf,Ci,YZ ab),(Nf,Ci,YZac),(Nf,Ci,YZad),(Nf,Ci,YZae),(Nf,Ci,YZaf),(Nf,Ci,YZag),(Nf,Cj,YZa),(Nf,Cj ,YZb),(Nf,Cj,YZc),(Nf,Cj,YZd),(Nf,Cj,YZe),(Nf,Cj,YZf),(Nf,Cj,YZg),(Nf,Cj,YZh),(Nf,Cj,Y Zi),(Nf,Cj,YZj),(Nf,Cj,YZk),(Nf,Cj,YZl),(Nf,Cj,YZm),(Nf,Cj,YZn),(Nf,Cj,YZo),(Nf,Cj,YZp) ,(Nf,Cj,YZq),(Nf,Cj,YZr),(Nf,Cj,YZs),(Nf,Cj,YZt),(Nf,Cj,YZu),(Nf,Cj,YZv),(Nf,Cj,YZw),( Nf,Cj,YZx),(Nf,Cj,YZy),(Nf,Cj,YZz),(Nf,Cj,YZaa),(Nf,Cj,YZab),(Nf,Cj,YZac),(Nf,Cj,YZad) ,(Nf,Cj,YZae),(Nf,Cj,YZaf),(Nf,Cj,YZag),(Ng,Ca,YZa),(Ng,Ca,YZb),(Ng,Ca,YZc),(Ng,Ca, YZd),(Ng,Ca,YZe),(Ng,Ca,YZf),(Ng,Ca,YZg),(Ng,Ca,YZh),(Ng,Ca,YZi),(Ng,Ca,YZj),(Ng, Ca,YZk),(Ng,Ca,YZl),(Ng,Ca,YZm),(Ng,Ca,YZn),(Ng,Ca,YZo),(Ng,Ca,YZp),(Ng,Ca,YZq), (Ng,Ca,YZr),(Ng,Ca,YZs),(Ng,Ca,YZt),(Ng,Ca,YZu),(Ng,Ca,YZv),(Ng,Ca,YZw),(Ng,Ca,Y Zx),(Ng,Ca,YZy),(Ng,Ca,YZz),(Ng,Ca,YZaa),(Ng,Ca,YZab),(Ng,Ca,YZac),(Ng,Ca,YZad),( Ng,Ca,YZae),(Ng,Ca,YZaf),(Ng,Ca,YZag),(Ng,Cb,YZa),(Ng,Cb,YZb),(Ng,Cb,YZc),(Ng,Cb, YZd),(Ng,Cb,YZe),(Ng,Cb,YZf),(Ng,Cb,YZg),(Ng,Cb,YZh),(Ng,Cb,YZi),(Ng,Cb,YZj),(Ng, Cb,YZk),(Ng,Cb,YZl),(Ng,Cb,YZm),(Ng,Cb,YZn),(Ng,Cb,YZo),(Ng,Cb,YZp),(Ng,Cb,YZq) ,(Ng,Cb,YZr),(Ng,Cb,YZs),(Ng,Cb,YZt),(Ng,Cb,YZu),(Ng,Cb,YZv),(Ng,Cb,YZw),(Ng,Cb, YZx),(Ng,Cb,YZy),(Ng,Cb,YZz),(Ng,Cb,YZaa),(Ng,Cb,YZab),(Ng,Cb,YZac),(Ng,Cb,YZad) ,(Ng,Cb,YZae),(Ng,Cb,YZaf),(Ng,Cb,YZag),(Ng,Cc,YZa),(Ng,Cc,YZb),(Ng,Cc,YZc),(Ng,C c,YZd),(Ng,Cc,YZe),(Ng,Cc,YZf),(Ng,Cc,YZg),(Ng,Cc,YZh),(Ng,Cc,YZi),(Ng,Cc,YZj),(Ng ,Cc,YZk),(Ng,Cc,YZl),(Ng,Cc,YZm),(Ng,Cc,YZn),(Ng,Cc,YZo),(Ng,Cc,YZp),(Ng,Cc,YZq), (Ng,Cc,YZr),(Ng,Cc,YZs),(Ng,Cc,YZt),(Ng,Cc,YZu),(Ng,Cc,YZv),(Ng,Cc,YZw),(Ng,Cc,Y Zx),(Ng,Cc,YZy),(Ng,Cc,YZz),(Ng,Cc,YZaa),(Ng,Cc,YZab),(Ng,Cc,YZac),(Ng,Cc,YZad),( Ng,Cc,YZae),(Ng,Cc,YZaf),(Ng,Cc,YZag),(Ng,Cd,YZa),(Ng,Cd,YZb),(Ng,Cd,YZc),(Ng,Cd, YZd),(Ng,Cd,YZe),(Ng,Cd,YZf),(Ng,Cd,YZg),(Ng,Cd,YZh),(Ng,Cd,YZi),(Ng,Cd,YZj),(Ng, Cd,YZk),(Ng,Cd,YZl),(Ng,Cd,YZm),(Ng,Cd,YZn),(Ng,Cd,YZo),(Ng,Cd,YZp),(Ng,Cd,YZq) ,(Ng,Cd,YZr),(Ng,Cd,YZs),(Ng,Cd,YZt),(Ng,Cd,YZu),(Ng,Cd,YZv),(Ng,Cd,YZw),(Ng,Cd, YZx),(Ng,Cd,YZy),(Ng,Cd,YZz),(Ng,Cd,YZaa),(Ng,Cd,YZab),(Ng,Cd,YZac),(Ng,Cd,YZad) ,(Ng,Cd,YZae),(Ng,Cd,YZaf),(Ng,Cd,YZag),(Ng,Ce,YZa),(Ng,Ce,YZb),(Ng,Ce,YZc),(Ng,C e,YZd),(Ng,Ce,YZe),(Ng,Ce,YZf),(Ng,Ce,YZg),(Ng,Ce,YZh),(Ng,Ce,YZi),(Ng,Ce,YZj),(Ng ,Ce,YZk),(Ng,Ce,YZl),(Ng,Ce,YZm),(Ng,Ce,YZn),(Ng,Ce,YZo),(Ng,Ce,YZp),(Ng,Ce,YZq), (Ng,Ce,YZr),(Ng,Ce,YZs),(Ng,Ce,YZt),(Ng,Ce,YZu),(Ng,Ce,YZv),(Ng,Ce,YZw),(Ng,Ce,Y Zx),(Ng,Ce,YZy),(Ng,Ce,YZz),(Ng,Ce,YZaa),(Ng,Ce,YZab),(Ng,Ce,YZac),(Ng,Ce,YZad),( Ng,Ce,YZae),(Ng,Ce,YZaf),(Ng,Ce,YZag),(Ng,Cf,YZa),(Ng,Cf,YZb),(Ng,Cf,YZc),(Ng,Cf,Y Zd),(Ng,Cf,YZe),(Ng,Cf,YZf),(Ng,Cf,YZg),(Ng,Cf,YZh),(Ng,Cf,YZi),(Ng,Cf,YZj),(Ng,Cf, YZk),(Ng,Cf,YZl),(Ng,Cf,YZm),(Ng,Cf,YZn),(Ng,Cf,YZo),(Ng,Cf,YZp),(Ng,Cf,YZq),(Ng, Cf,YZr),(Ng,Cf,YZs),(Ng,Cf,YZt),(Ng,Cf,YZu),(Ng,Cf,YZv),(Ng,Cf,YZw),(Ng,Cf,YZx),(N g,Cf,YZy),(Ng,Cf,YZz),(Ng,Cf,YZaa),(Ng,Cf,YZab),(Ng,Cf,YZac),(Ng,Cf,YZad),(Ng,Cf,Y Zae),(Ng,Cf,YZaf),(Ng,Cf,YZag),(Ng,Cg,YZa),(Ng,Cg,YZb),(Ng,Cg,YZc),(Ng,Cg,YZd),(N g,Cg,YZe),(Ng,Cg,YZf),(Ng,Cg,YZg),(Ng,Cg,YZh),(Ng,Cg,YZi),(Ng,Cg,YZj),(Ng,Cg,Ya) ,(Ng,Cg,YZl),(Ng,Cg,YZm),(Ng,Cg,YZn),(Ng,Cg,YZo),(Ng,Cg,YZp),(Ng,Cg,YZq),(Ng,Cg, YZr),(Ng,Cg,YZs),(Ng,Cg,YZt),(Ng,Cg,YZu),(Ng,Cg,YZv),(Ng,Cg,YZw),(Ng,Cg,YZx),(Ng ,Cg,YZy),(Ng,Cg,YZz),(Ng,Cg,YZaa),(Ng,Cg,YZab),(Ng,Cg,YZac),(Ng,Cg,YZad),(Ng,Cg, YZae),(Ng,Cg,YZaf),(Ng,Cg,YZag),(Ng,Ch,YZa),(Ng,Ch,YZb),(Ng,Ch,YZc),(Ng,Ch,YZd),( Ng,Ch,YZe),(Ng,Ch,YZf),(Ng,Ch,YZg),(Ng,Ch,Ya),(Ng,Ch,YZi),(Ng,Ch,YZj),(Ng,Ch,YZ k),(Ng,Ch,YZl),(Ng,Ch,YZm),(Ng,Ch,YZn),(Ng,Ch,YZo),(Ng,Ch,YZp),(Ng,Ch,YZq),(Ng,C h,YZr),(Ng,Ch,YZs),(Ng,Ch,YZt),(Ng,Ch,YZu),(Ng,Ch,YZv),(Ng,Ch,YZw),(Ng,Ch,YZx),( Ng,Ch,YZy),(Ng,Ch,YZz),(Ng,Ch,YZaa),(Ng,Ch,YZab),(Ng,Ch,YZac),(Ng,Ch,YZad),(Ng,C h,YZae),(Ng,Ch,YZaf),(Ng,Ch,YZag),(Ng,Ci,YZa),(Ng,Ci,YZb),(Ng,Ci,YZc),(Ng,Ci,YZd),( Ng,Ci,YZe),(Ng,Ci,YZf),(Ng,Ci,YZg),(Ng,Ci,YZh),(Ng,Ci,YZi),(Ng,Ci,YZj),(Ng,Ci,YZk),( Ng,Ci,YZl),(Ng,Ci,YZm),(Ng,Ci,YZn),(Ng,Ci,YZo),(Ng,Ci,YZp),(Ng,Ci,YZq),(Ng,Ci,YZr), (Ng,Ci,YZs),(Ng,Ci,YZt),(Ng,Ci,YZu),(Ng,Ci,YZv),(Ng,Ci,YZw),(Ng,Ci,YZx),(Ng,Ci,YZy) ,(Ng,Ci,YZz),(Ng,Ci,YZaa),(Ng,Ci,YZab),(Ng,Ci,YZac),(Ng,Ci,YZad),(Ng,Ci,YZae),(Ng,Ci ,YZaf),(Ng,Ci,YZag),(Ng,Cj,YZa),(Ng,Cj,YZb),(Ng,Cj,YZc),(Ng,Cj,YZd),(Ng,Cj,YZe),(Ng, Cj,YZf),(Ng,Cj,YZg),(Ng,Cj,YZh),(Ng,Cj,YZi),(Ng,Cj,YZj),(Ng,Cj,Ya),(Ng,Cj,YZl),(Ng, Cj,YZm),(Ng,Cj,YZn),(Ng,Cj,YZo),(Ng,Cj,YZp),(Ng,Cj,YZq),(Ng,Cj,YZr),(Ng,Cj,YZs),(N g,Cj,YZt),(Ng,Cj,YZu),(Ng,Cj,YZv),(Ng,Cj,YZw),(Ng,Cj,YZx),(Ng,Cj,YZy),(Ng,Cj,YZz),( Ng,Cj,YZaa),(Ng,Cj,YZab),(Ng,Cj,YZac),(Ng,Cj,YZad),(Ng,Cj,YZae),(Ng,Cj,YZaf),(Ng,Cj, YZag),(Nh,Ca,YZa),(Nh,Ca,YZb),(Nh,Ca,YZc),(Nh,Ca,YZd),(Nh,Ca,YZe),(Nh,Ca,YZf),(Nh ,Ca,YZg),(Nh,Ca,YZh),(Nh,Ca,YZi),(Nh,Ca,YZj),(Nh,Ca,YZk),(Nh,Ca,YZl),(Nh,Ca,YZm),( Nh,Ca,YZn),(Nh,Ca,YZo),(Nh,Ca,YZp),(Nh,Ca,YZq),(Nh,Ca,YZr),(Nh,Ca,YZs),(Nh,Ca,YZ t),(Nh,Ca,YZu),(Nh,Ca,YZv),(Nh,Ca,YZw),(Nh,Ca,YZx),(Nh,Ca,YZy),(Nh,Ca,YZz),(Nh,Ca ,YZaa),(Nh,Ca,YZab),(Nh,Ca,YZac),(Nh,Ca,YZad),(Nh,Ca,YZae),(Nh,Ca,YZaf),(Nh,Ca,YZ ag),(Nh,Cb,YZa),(Nh,Cb,YZb),(Nh,Cb,YZc),(Nh,Cb,YZd),(Nh,Cb,YZe),(Nh,Cb,YZf),(Nh,C b,YZg),(Nh,Cb,YZh),(Nh,Cb,YZi),(Nh,Cb,YZj),(Nh,Cb,Ya),(Nh,Cb,YZl),(Nh,Cb,YZm),( Nh,Cb,YZn),(Nh,Cb,YZo),(Nh,Cb,YZp),(Nh,Cb,YZq),(Nh,Cb,YZr),(Nh,Cb,YZs),(Nh,Cb,Y Zt),(Nh,Cb,YZu),(Nh,Cb,YZv),(Nh,Cb,YZw),(Nh,Cb,YZx),(Nh,Cb,YZy),(Nh,Cb,YZz),(Nh, Cb,YZaa),(Nh,Cb,YZab),(Nh,Cb,YZac),(Nh,Cb,YZad),(Nh,Cb,YZae),(Nh,Cb,YZaf),(Nh,Cb, YZag),(Nh,Cc,YZa),(Nh,Cc,YZb),(Nh,Cc,YZc),(Nh,Cc,YZd),(Nh,Cc,YZe),(Nh,Cc,YZf),(Nh ,Cc,YZg),(Nh,Cc,YZh),(Nh,Cc,YZi),(Nh,Cc,YZj),(Nh,Cc,YZk),(Nh,Cc,YZl),(Nh,Cc,YZm),( Nh,Cc,YZn),(Nh,Cc,YZo),(Nh,Cc,YZp),(Nh,Cc,YZq),(Nh,Cc,YZr),(Nh,Cc,YZs),(Nh,Cc,YZ t),(Nh,Cc,YZu),(Nh,Cc,YZv),(Nh,Cc,YZw),(Nh,Cc,YZx),(Nh,Cc,YZy),(Nh,Cc,YZz),(Nh,Cc ,YZaa),(Nh,Cc,YZab),(Nh,Cc,YZac),(Nh,Cc,YZad),(Nh,Cc,YZae),(Nh,Cc,YZaf),(Nh,Cc,YZ ag),(Nh,Cd,YZa),(Nh,Cd,YZb),(Nh,Cd,YZc),(Nh,Cd,YZd),(Nh,Cd,YZe),(Nh,Cd,YZf),(Nh,C d,YZg),(Nh,Cd,YZh),(Nh,Cd,YZi),(Nh,Cd,YZj),(Nh,Cd,YZk),(Nh,Cd,YZl),(Nh,Cd,YZm),( Nh,Cd,YZn),(Nh,Cd,YZo),(Nh,Cd,YZp),(Nh,Cd,YZq),(Nh,Cd,YZr),(Nh,Cd,YZs),(Nh,Cd,Y Zt),(Nh,Cd,YZu),(Nh,Cd,YZv),(Nh,Cd,YZw),(Nh,Cd,YZx),(Nh,Cd,YZy),(Nh,Cd,YZz),(Nh, Cd,YZaa),(Nh,Cd,YZab),(Nh,Cd,YZac),(Nh,Cd,YZad),(Nh,Cd,YZae),(Nh,Cd,YZaf),(Nh,Cd, YZag),(Nh,Ce,YZa),(Nh,Ce,YZb),(Nh,Ce,YZc),(Nh,Ce,YZd),(Nh,Ce,YZe),(Nh,Ce,YZf),(Nh ,Ce,YZg),(Nh,Ce,YZh),(Nh,Ce,YZi),(Nh,Ce,YZj),(Nh,Ce,YZk),(Nh,Ce,YZl),(Nh,Ce,YZm),( Nh,Ce,YZn),(Nh,Ce,YZo),(Nh,Ce,YZp),(Nh,Ce,YZq),(Nh,Ce,YZr),(Nh,Ce,YZs),(Nh,Ce,YZ t),(Nh,Ce,YZu),(Nh,Ce,YZv),(Nh,Ce,YZw),(Nh,Ce,YZx),(Nh,Ce,YZy),(Nh,Ce,YZz),(Nh,Ce ,YZaa),(Nh,Ce,YZab),(Nh,Ce,YZac),(Nh,Ce,YZad),(Nh,Ce,YZae),(Nh,Ce,YZaf),(Nh,Ce,YZ ag),(Nh,Cf,YZa),(Nh,Cf,YZb),(Nh,Cf,YZc),(Nh,Cf,YZd),(Nh,Cf,YZe),(Nh,Cf,YZf),(Nh,Cf, YZg),(Nh,Cf,YZh),(Nh,Cf,YZi),(Nh,Cf,YZj),(Nh,Cf,YZk),(Nh,Cf,YZl),(Nh,Cf,YZm),(Nh,C f,YZn),(Nh,Cf,YZo),(Nh,Cf,YZp),(Nh,Cf,YZq),(Nh,Cf,YZr),(Nh,Cf,YZs),(Nh,Cf,YZt),(Nh, Cf,YZu),(Nh,Cf,YZv),(Nh,Cf,YZw),(Nh,Cf,YZx),(Nh,Cf,YZy),(Nh,Cf,YZz),(Nh,Cf,YZaa),( Nh,Cf,YZab),(Nh,Cf,YZac),(Nh,Cf,YZad),(Nh,Cf,YZae),(Nh,Cf,YZaf),(Nh,Cf,YZag),(Nh,C g,YZa),(Nh,Cg,YZb),(Nh,Cg,YZc),(Nh,Cg,YZd),(Nh,Cg,YZe),(Nh,Cg,YZf),(Nh,Cg,YZg),( Nh,Cg,YZh),(Nh,Cg,YZi),(Nh,Cg,YZj),(Nh,Cg,YZk),(Nh,Cg,YZl),(Nh,Cg,YZm),(Nh,Cg,Y Zn),(Nh,Cg,YZo),(Nh,Cg,YZp),(Nh,Cg,YZq),(Nh,Cg,YZr),(Nh,Cg,YZs),(Nh,Cg,YZt),(Nh,C g,YZu),(Nh,Cg,YZv),(Nh,Cg,YZw),(Nh,Cg,YZx),(Nh,Cg,YZy),(Nh,Cg,YZz),(Nh,Cg,YZaa), (Nh,Cg,YZab),(Nh,Cg,YZac),(Nh,Cg,YZad),(Nh,Cg,YZae),(Nh,Cg,YZaf),(Nh,Cg,YZag),(N h,Ch,YZa),(Nh,Ch,YZb),(Nh,Ch,YZc),(Nh,Ch,YZd),(Nh,Ch,YZe),(Nh,Ch,YZf),(Nh,Ch,YZg) ,(Nh,Ch,YZh),(Nh,Ch,YZi),(Nh,Ch,YZj),(Nh,Ch,YZl),(Nh,Ch,YZl),(Nh,Ch,YZm),(Nh,Ch, YZn),(Nh,Ch,YZo),(Nh,Ch,YZp),(Nh,Ch,YZq),(Nh,Ch,YZr),(Nh,Ch,YZs),(Nh,Ch,YZt),(Nh, Ch,YZu),(Nh,Ch,YZv),(Nh,Ch,YZw),(Nh,Ch,YZx),(Nh,Ch,YZy),(Nh,Ch,YZz),(Nh,Ch,YZa a),(Nh,Ch,YZab),(Nh,Ch,YZac),(Nh,Ch,YZad),(Nh,Ch,YZae),(Nh,Ch,YZaf),(Nh,Ch,YZag),( Nh,Ci,YZa),(Nh,Ci,YZb),(Nh,Ci,YZc),(Nh,Ci,YZd),(Nh,Ci,YZe),(Nh,Ci,YZf),(Nh,Ci,YZg),( Nh,Ci,YZh),(Nh,Ci,YZi),(Nh,Ci,YZj),(Nh,Ci,YZk),(Nh,Ci,YZl),(Nh,Ci,YZm),(Nh,Ci,YZn),( Nh,Ci,YZo),(Nh,Ci,YZp),(Nh,Ci,YZq),(Nh,Ci,YZr),(Nh,Ci,YZs),(Nh,Ci,YZt),(Nh,Ci,YZu),( Nh,Ci,YZv),(Nh,Ci,YZw),(Nh,Ci,YZx),(Nh,Ci,YZy),(Nh,Ci,YZz),(Nh,Ci,YZaa),(Nh,Ci,YZa b),(Nh,Ci,YZac),(Nh,Ci,YZad),(Nh,Ci,YZae),(Nh,Ci,YZaf),(Nh,Ci,YZag),(Nh,Cj,YZa),(Nh, Cj,YZb),(Nh,Cj,YZc),(Nh,Cj,YZd),(Nh,Cj,YZe),(Nh,Cj,YZf),(Nh,Cj,YZg),(Nh,Cj,YZh),(Nh ,Cj,YZi),(Nh,Cj,YZj),(Nh,Cj,YZk),(Nh,Cj,YZl),(Nh,Cj,YZm),(Nh,Cj,YZn),(Nh,Cj,YZo),(Nh ,Cj,YZp),(Nh,Cj,YZq),(Nh,Cj,YZr),(Nh,Cj,YZs),(Nh,Cj,YZt),(Nh,Cj,YZu),(Nh,Cj,YZv),(Nh ,Cj,YZw),(Nh,Cj,YZx),(Nh,Cj,YZy),(Nh,Cj,YZz),(Nh,Cj,YZaa),(Nh,Cj,YZab),(Nh,Cj,YZac) ,(Nh,Cj,YZad),(Nh,Cj,YZae),(Nh,Cj,YZaf),(Nh,Cj,YZag),(Ni,Ca,YZa),(Ni,Ca,YZb),(Ni,Ca, YZc),(Ni,Ca,YZd),(Ni,Ca,YZe),(Ni,Ca,YZf),(Ni,Ca,YZg),(Ni,Ca,YZh),(Ni,Ca,YZi),(Ni,Ca, YZj),(Ni,Ca,YZk),(Ni,Ca,YZl),(Ni,Ca,YZm),(Ni,Ca,YZn),(Ni,Ca,YZo),(Ni,Ca,YZp),(Ni,Ca, YZq),(Ni,Ca,YZr),(Ni,Ca,YZs),(Ni,Ca,YZt),(Ni,Ca,YZu),(Ni,Ca,YZv),(Ni,Ca,YZw),(Ni,Ca, YZx),(Ni,Ca,YZy),(Ni,Ca,YZz),(Ni,Ca,YZaa),(Ni,Ca,YZab),(Ni,Ca,YZac),(Ni,Ca,YZad),(Ni ,Ca,YZae),(Ni,Ca,YZaf),(Ni,Ca,YZag),(Ni,Cb,YZa),(Ni,Cb,YZb),(Ni,Cb,YZc),(Ni,Cb,YZd), (Ni,Cb,YZe),(Ni,Cb,YZf),(Ni,Cb,YZg),(Ni,Cb,YZl),(Ni,Cb,YZi),(Ni,Cb,YZj),(Ni,Cb,YZk),( Ni,Cb,YZl),(Ni,Cb,YZm),(Ni,Cb,YZn),(Ni,Cb,YZo),(Ni,Cb,YZp),(Ni,Cb,YZq),(Ni,Cb,YZr), (Ni,Cb,YZs),(Ni,Cb,YZt),(Ni,Cb,YZu),(Ni,Cb,YZv),(Ni,Cb,YZw),(Ni,Cb,YZx),(Ni,Cb,YZy) ,(Ni,Cb,YZz),(Ni,Cb,YZaa),(Ni,Cb,YZab),(Ni,Cb,YZac),(Ni,Cb,YZad),(Ni,Cb,YZae),(Ni,Cb ,YZaf),(Ni,Cb,YZag),(Ni,Cc,YZa),(Ni,Cc,YZb),(Ni,Cc,YZc),(Ni,Cc,YZd),(Ni,Cc,YZe),(Ni, Cc,YZf),(Ni,Cc,YZg),(Ni,Cc,YZh),(Ni,Cc,YZi),(Ni,Cc,YZj),(Ni,Cc,Ya),(Ni,Cc,YZl),(Ni,C c,YZm),(Ni,Cc,YZn),(Ni,Cc,YZo),(Ni,Cc,YZp),(Ni,Cc,YZq),(Ni,Cc,YZr),(Ni,Cc,YZs),(Ni,C c,YZt),(Ni,Cc,YZu),(Ni,Cc,YZv),(Ni,Cc,YZw),(Ni,Cc,YZx),(Ni,Cc,YZy),(Ni,Cc,YZz),(Ni,C c,YZaa),(Ni,Cc,YZab),(Ni,Cc,YZac),(Ni,Cc,YZad),(Ni,Cc,YZae),(Ni,Cc,YZaf),(Ni,Cc,YZag) ,(Ni,Cd,YZa),(Ni,Cd,YZb),(Ni,Cd,YZc),(Ni,Cd,YZd),(Ni,Cd,YZe),(Ni,Cd,YZf),(Ni,Cd,YZg) ,(Ni,Cd,YZh),(Ni,Cd,YZi),(Ni,Cd,YZj),(Ni,Cd,YZk),(Ni,Cd,YZl),(Ni,Cd,YZm),(Ni,Cd,YZn) ,(Ni,Cd,YZo),(Ni,Cd,YZp),(Ni,Cd,YZq),(Ni,Cd,YZr),(Ni,Cd,YZs),(Ni,Cd,YZt),(Ni,Cd,YZu) ,(Ni,Cd,YZv),(Ni,Cd,YZw),(Ni,Cd,YZx),(Ni,Cd,YZy),(Ni,Cd,YZz),(Ni,Cd,YZaa),(Ni,Cd,Y Zab),(Ni,Cd,YZac),(Ni,Cd,YZad),(Ni,Cd,YZae),(Ni,Cd,YZaf),(Ni,Cd,YZag),(Ni,Ce,YZa),(N i,Ce,YZb),(Ni,Ce,YZc),(Ni,Ce,YZd),(Ni,Ce,YZe),(Ni,Ce,YZf),(Ni,Ce,YZg),(Ni,Ce,YZh),(Ni ,Ce,YZi),(Ni,Ce,YZj),(Ni,Ce,YZk),(Ni,Ce,YZl),(Ni,Ce,YZm),(Ni,Ce,YZn),(Ni,Ce,YZo),(Ni, Ce,YZp),(Ni,Ce,YZq),(Ni,Ce,YZr),(Ni,Ce,YZs),(Ni,Ce,YZt),(Ni,Ce,YZu),(Ni,Ce,YZv),(Ni, Ce,YZw),(Ni,Ce,YZx),(Ni,Ce,YZy),(Ni,Ce,YZz),(Ni,Ce,YZaa),(Ni,Ce,YZab),(Ni,Ce,YZac), (Ni,Ce,YZad),(Ni,Ce,YZae),(Ni,Ce,YZaf),(Ni,Ce,YZag),(Ni,Cf,YZa),(Ni,Cf,YZb),(Ni,Cf,YZ c),(Ni,Cf,YZd),(Ni,Cf,YZe),(Ni,Cf,YZf),(Ni,Cf,YZg),(Ni,Cf,YZh),(Ni,Cf,YZi),(Ni,Cf,YZj),( Ni,Cf,YZk),(Ni,Cf,YZl),(Ni,Cf,YZm),(Ni,Cf,YZn),(Ni,Cf,YZo),(Ni,Cf,YZp),(Ni,Cf,YZq),(N i,Cf,YZr),(Ni,Cf,YZs),(Ni,Cf,YZt),(Ni,Cf,YZu),(Ni,Cf,YZv),(Ni,Cf,YZw),(Ni,Cf,YZx),(Ni, Cf,YZy),(Ni,Cf,YZz),(Ni,Cf,YZaa),(Ni,Cf,YZab),(Ni,Cf,YZac),(Ni,Cf,YZad),(Ni,Cf,YZae),( Ni,Cf,YZaf),(Ni,Cf,YZag),(Ni,Cg,YZa),(Ni,Cg,YZb),(Ni,Cg,YZc),(Ni,Cg,YZd),(Ni,Cg,YZe) ,(Ni,Cg,YZf),(Ni,Cg,YZg),(Ni,Cg,YZh),(Ni,Cg,YZi),(Ni,Cg,YZj),(Ni,Cg,YZk),(Ni,Cg,YZl), (Ni,Cg,YZm),(Ni,Cg,YZn),(Ni,Cg,YZo),(Ni,Cg,YZp),(Ni,Cg,YZq),(Ni,Cg,YZr),(Ni,Cg,YZs) ,(Ni,Cg,YZt),(Ni,Cg,YZu),(Ni,Cg,YZv),(Ni,Cg,YZw),(Ni,Cg,YZx),(Ni,Cg,YZy),(Ni,Cg,YZ z),(Ni,Cg,YZaa),(Ni,Cg,YZab),(Ni,Cg,YZac),(Ni,Cg,YZad),(Ni,Cg,YZae),(Ni,Cg,YZaf),(Ni, Cg,YZag),(Ni,Ch,YZa),(Ni,Ch,YZb),(Ni,Ch,YZc),(Ni,Ch,YZd),(Ni,Ch,YZe),(Ni,Ch,YZf),(N i,Ch,YZg),(Ni,Ch,YZh),(Ni,Ch,YZi),(Ni,Ch,YZj),(Ni,Ch,YZk),(Ni,Ch,YZl),(Ni,Ch,YZm),(N i,Ch,YZn),(Ni,Ch,YZo),(Ni,Ch,YZp),(Ni,Ch,YZq),(Ni,Ch,YZr),(Ni,Ch,YZs),(Ni,Ch,YZt),(N i,Ch,YZu),(Ni,Ch,YZv),(Ni,Ch,YZw),(Ni,Ch,YZx),(Ni,Ch,YZy),(Ni,Ch,YZz),(Ni,Ch,YZaa), (Ni,Ch,YZab),(Ni,Ch,YZac),(Ni,Ch,YZad),(Ni,Ch,YZae),(Ni,Ch,YZaf),(Ni,Ch,YZag),(Ni,Ci ,YZa),(Ni,Ci,YZb),(Ni,Ci,YZc),(Ni,Ci,YZd),(Ni,Ci,YZe),(Ni,Ci,YZf),(Ni,Ci,YZg),(Ni,Ci,YZ h),(Ni,Ci,YZi),(Ni,Ci,YZj),(Ni,Ci,YZk),(Ni,Ci,YZl),(Ni,Ci,YZm),(Ni,Ci,YZn),(Ni,Ci,YZo),( Ni,Ci,YZp),(Ni,Ci,YZq),(Ni,Ci,YZr),(Ni,Ci,YZs),(Ni,Ci,YZt),(Ni,Ci,YZu),(Ni,Ci,YZv),(Ni, Ci,YZw),(Ni,Ci,YZx),(Ni,Ci,YZy),(Ni,Ci,YZz),(Ni,Ci,YZaa),(Ni,Ci,YZab),(Ni,Ci,YZac),(Ni ,Ci,YZad),(Ni,Ci,YZae),(Ni,Ci,YZaf),(Ni,Ci,YZag),(Ni,Cj,YZa),(Ni,Cj,YZb),(Ni,Cj,YZc),(N i,Cj,YZd),(Ni,Cj,YZe),(Ni,Cj,YZf),(Ni,Cj,YZg),(Ni,Cj,YZh),(Ni,Cj,YZi),(Ni,Cj,YZj),(Ni,Cj, YZk),(Ni,Cj,YZl),(Ni,Cj,YZm),(Ni,Cj,YZn),(Ni,Cj,YZo),(Ni,Cj,YZp),(Ni,Cj,YZq),(Ni,Cj,Y Zr),(Ni,Cj,YZs),(Ni,Cj,YZt),(Ni,Cj,YZu),(Ni,Cj,YZv),(Ni,Cj,YZw),(Ni,Cj,YZx),(Ni,Cj,YZy) ,(Ni,Cj,YZz),(Ni,Cj,YZaa),(Ni,Cj,YZab),(Ni,Cj,YZac),(Ni,Cj,YZad),(Ni,Cj,YZae),(Ni,Cj,YZ af),(Ni,Cj,YZag),(Nj,Ca,YZa),(Nj,Ca,YZb),(Nj,Ca,YZc),(Nj,Ca,YZd),(Nj,Ca,YZe),(Nj,Ca,Y Zf),(Nj,Ca,YZg),(Nj,Ca,YZh),(Nj,Ca,YZi),(Nj,Ca,YZj),(Nj,Ca,YZk),(Nj,Ca,YZl),(Nj,Ca,YZ m),(Nj,Ca,YZn),(Nj,Ca,YZo),(Nj,Ca,YZp),(Nj,Ca,YZq),(Nj,Ca,YZr),(Nj,Ca,YZs),(Nj,Ca,YZ t),(Nj,Ca,YZu),(Nj,Ca,YZv),(Nj,Ca,YZw),(Nj,Ca,YZx),(Nj,Ca,YZy),(Nj,Ca,YZz),(Nj,Ca,YZ aa),(Nj,Ca,YZab),(Nj,Ca,YZac),(Nj,Ca,YZad),(Nj,Ca,YZae),(Nj,Ca,YZaf),(Nj,Ca,YZag),(Nj, Cb,YZa),(Nj,Cb,YZb),(Nj,Cb,YZc),(Nj,Cb,YZd),(Nj,Cb,YZe),(Nj,Cb,YZf),(Nj,Cb,YZg),(Nj, Cb,YZh),(Nj,Cb,YZi),(Nj,Cb,YZj),(Nj,Cb,YZk),(Nj,Cb,YZl),(Nj,Cb,YZm),(Nj,Cb,YZn),(Nj, Cb,YZo),(Nj,Cb,YZp),(Nj,Cb,YZq),(Nj,Cb,YZr),(Nj,Cb,YZs),(Nj,Cb,YZt),(Nj,Cb,YZu),(Nj, Cb,YZv),(Nj,Cb,YZw),(Nj,Cb,YZx),(Nj,Cb,YZy),(Nj,Cb,YZz),(Nj,Cb,YZaa),(Nj,Cb,YZab), (Nj,Cb,YZac),(Nj,Cb,YZad),(Nj,Cb,YZae),(Nj,Cb,YZaf),(Nj,Cb,YZag),(Nj,Cc,YZa),(Nj,Cc, YZb),(Nj,Cc,YZc),(Nj,Cc,YZd),(Nj,Cc,YZe),(Nj,Cc,YZf),(Nj,Cc,YZg),(Nj,Cc,YZh),(Nj,Cc, YZi),(Nj,Cc,YZj),(Nj,Cc,YZk),(Nj,Cc,YZl),(Nj,Cc,YZm),(Nj,Cc,YZn),(Nj,Cc,YZo),(Nj,Cc, YZp),(Nj,Cc,YZq),(Nj,Cc,YZr),(Nj,Cc,YZs),(Nj,Cc,YZt),(Nj,Cc,YZu),(Nj,Cc,YZv),(Nj,Cc, YZw),(Nj,Cc,YZx),(Nj,Cc,YZy),(Nj,Cc,YZz),(Nj,Cc,YZaa),(Nj,Cc,YZab),(Nj,Cc,YZac),(Nj, Cc,YZad),(Nj,Cc,YZae),(Nj,Cc,YZaf),(Nj,Cc,YZag),(Nj,Cd,YZa),(Nj,Cd,YZb),(Nj,Cd,YZc), (Nj,Cd,YZd),(Nj,Cd,YZe),(Nj,Cd,YZf),(Nj,Cd,YZg),(Nj,Cd,YZh),(Nj,Cd,YZi),(Nj,Cd,YZj),( Nj,Cd,YZk),(Nj,Cd,YZl),(Nj,Cd,YZm)_{;}(Nj,Cd,YZn),(Nj,Cd,YZo),(Nj,Cd,YZp),(Nj,Cd,YZq) ,(Nj,Cd,YZr),(Nj,Cd,YZs),(Nj,Cd,YZt),(Nj,Cd,YZu),(Nj,Cd,YZv),(Nj,Cd,YZw),(Nj,Cd,YZx) ,(Nj,Cd,YZy),(Nj,Cd,YZz),(Nj,Cd,YZaa),(Nj,Cd,YZab),(Nj,Cd,YZac),(Nj,Cd,YZad),(Nj,Cd ,YZae),(Nj,Cd,YZaf),(Nj,Cd,YZag),(Nj,Ce,YZa),(Nj,Ce,YZb),(Nj,Ce,YZc),(Nj,Ce,YZd),(Nj, Ce,YZe),(Nj,Ce,YZf),(Nj,Ce,YZg),(Nj,Ce,YZh),(Nj,Ce,YZi),(Nj,Ce,YZj),(Nj,Ce,YZk),(Nj,C e,YZl),(Nj,Ce,YZm),(Nj,Ce,YZn),(Nj,Ce,YZo),(Nj,Ce,YZp),(Nj,Ce,YZq),(Nj,Ce,YZr),(Nj,C e,YZs),(Nj,Ce,YZt),(Nj,Ce,YZu),(Nj,Ce,YZv),(Nj,Ce,YZw),(Nj,Ce,YZx),(Nj,Ce,YZy),(Nj,C e,YZz),(Nj,Ce,YZaa),(Nj,Ce,YZab),(Nj,Ce,YZac),(Nj,Ce,YZad),(Nj,Ce,YZae),(Nj,Ce,YZaf), (Nj,Ce,YZag),(Nj,Cf,YZa),(Nj,Cf,YZb),(Nj,Cf,YZc),(Nj,Cf,YZd),(Nj,Cf,YZe),(Nj,Cf,YZf),( Nj,Cf,YZg),(Nj,Cf,YZh),(Nj,Cf,YZi),(Nj,Cf,YZj),(Nj,Cf,YZk),(Nj,Cf,YZl),(Nj,Cf,YZm),(Nj, Cf,YZn),(Nj,Cf,YZo),(Nj,Cf,YZp),(Nj,Cf,YZq),(Nj,Cf,YZr),(Nj,Cf,YZs),(Nj,Cf,YZt),(Nj,Cf, YZu),(Nj,Cf,YZv),(Nj,Cf,YZw),(Nj,Cf,YZx),(Nj,Cf,YZy),(Nj,Cf,YZz),(Nj,Cf,YZaa),(Nj,Cf, YZab),(Nj,Cf,YZac),(Nj,Cf,YZad),(Nj,Cf,YZae),(Nj,Cf,YZaf),(Nj,Cf,YZag),(Nj,Cg,YZa),(N j,Cg,YZb),(Nj,Cg,YZc),(Nj,Cg,YZd),(Nj,Cg,YZe),(Nj,Cg,YZf),(Nj,Cg,YZg),(Nj,Cg,YZh),( Nj,Cg,YZi),(Nj,Cg,YZj),(Nj,Cg,YZk),(Nj,Cg,YZl),(Nj,Cg,YZm),(Nj,Cg,YZn),(Nj,Cg,YZo),( Nj,Cg,YZp),(Nj,Cg,YZq),(Nj,Cg,YZr),(Nj,Cg,YZs),(Nj,Cg,YZt),(Nj,Cg,YZu),(Nj,Cg,YZv),( Nj,Cg,YZw),(Nj,Cg,YZx),(Nj,Cg,YZy),(Nj,Cg,YZz),(Nj,Cg,YZaa),(Nj,Cg,YZab),(Nj,Cg,YZ ac),(Nj,Cg,YZad),(Nj,Cg,YZae),(Nj,Cg,YZaf),(Nj,Cg,YZag),(Nj,Ch,YZa),(Nj,Ch,YZb),(Nj, Ch,YZc),(Nj,Ch,YZd),(Nj,Ch,YZe),(Nj,Ch,YZf),(Nj,Ch,YZg),(Nj,Ch,YZh),(Nj,Ch,YZi),(Nj, Ch,YZj),(Nj,Ch,YZk),(Nj,Ch,YZl),(Nj,Ch,YZm),(Nj,Ch,YZn),(Nj,Ch,YZo),(Nj,Ch,YZp),(Nj ,Ch,YZq),(Nj,Ch,YZr),(Nj,Ch,YZs),(Nj,Ch,YZt),(Nj,Ch,YZu),(Nj,Ch,YZv),(Nj,Ch,YZw),(N j,Ch,YZx),(Nj,Ch,YZy),(Nj,Ch,YZz),(Nj,Ch,YZaa),(Nj,Ch,YZab),(Nj,Ch,YZac),(Nj,Ch,YZa d),(Nj,Ch,YZae),(Nj,Ch,YZaf),(Nj,Ch,YZag),(Nj,Ci,YZa),(Nj,Ci,YZb),(Nj,Ci,YZc),(Nj,Ci,Y Zd),(Nj,Ci,YZe),(Nj,Ci,YZf),(Nj,Ci,YZg),(Nj,Ci,YZh),(Nj,Ci,YZi),(Nj,Ci,YZj),(Nj,Ci,YZk), (Nj,Ci,YZl),(Nj,Ci,YZm),(Nj,Ci,YZn),(Nj,Ci,YZo),(Nj,Ci,YZp),(Nj,Ci,YZq),(Nj,Ci,YZr),(Nj ,Ci,YZs),(Nj,Ci,YZt),(Nj,Ci,YZu),(Nj,Ci,YZv),(Nj,Ci,YZw),(Nj,Ci,YZx),(Nj,Ci,YZy),(Nj,Ci ,YZz),(Nj,Ci,YZaa),(Nj,Ci,YZab),(Nj,Ci,YZac),(Nj,Ci,YZad),(Nj,Ci,YZae),(Nj,Ci,YZaf),(Nj ,Ci,YZag),(Nj,Cj,YZa),(Nj,Cj,YZb),(Nj,Cj,YZc),(Nj,Cj,YZd),(Nj,Cj,YZe),(Nj,Cj,YZf),(Nj,C j,YZg),(Nj,Cj,YZh),(Nj,Cj,YZi),(Nj,Cj,YZj),(Nj,Cj,YZk),(Nj,Cj,YZl),(Nj,Cj,YZm),(Nj,Cj,Y Zn),(Nj,Cj,YZo),(Nj,Cj,YZp),(Nj,Cj,YZq),(Nj,Cj,YZr),(Nj,Cj,YZs),(Nj,Cj,YZt),(Nj,Cj,YZu), (Nj,Cj,YZv),(Nj,Cj,YZw),(Nj,Cj,YZx),(Nj,Cj,YZy),(Nj,Cj,YZz),(Nj,Cj,YZaa),(Nj,Cj,YZab), (Nj,Cj,YZac),(Nj,Cj,YZad),(Nj,Cj,YZae),(Nj,Cj,YZaf),(Nj,Cj,YZag),(Nk,Ca,YZa),(Nk,Ca,Y Zb),(Nk,Ca,YZc),(Nk,Ca,YZd),(Nk,Ca,YZe),(Nk,Ca,YZf),(Nk,Ca,YZg),(Nk,Ca,YZh),(Nk,C a,YZi),(Nk,Ca,YZj),(Nk,Ca,YZk),(Nk,Ca,YZl),(Nk,Ca,YZm),(Nk,Ca,YZn),(Nk,Ca,YZo),(N k,Ca,YZp),(Nk,Ca,YZq),(Nk,Ca,YZr),(Nk,Ca,YZs),(Nk,Ca,YZt),(Nk,Ca,YZu),(Nk,Ca,YZv), (Nk,Ca,YZw),(Nk,Ca,YZx),(Nk,Ca,YZy),(Nk,Ca,YZz),(Nk,Ca,YZaa),(Nk,Ca,YZab),(Nk,Ca ,YZac),(Nk,Ca,YZad),(Nk,Ca,YZae),(Nk,Ca,YZaf),(Nk,Ca,YZag),(Nk,Cb,YZa),(Nk,Cb,YZb) ,(Nk,Cb,YZc),(Nk,Cb,YZd),(Nk,Cb,YZe),(Nk,Cb,YZf),(Nk,Cb,YZg),(Nk,Cb,YZh),(Nk,Cb, YZi),(Nk,Cb,YZj),(Nk,Cb,YZk),(Nk,Cb,YZl),(Nk,Cb,YZm),(Nk,Cb,YZn),(Nk,Cb,YZo),(Nk ,Cb,YZp),(Nk,Cb,YZq),(Nk,Cb,YZr),(Nk,Cb,YZs),(Nk,Cb,YZt),(Nk,Cb,YZu),(Nk,Cb,YZv), (Nk,Cb,YZw),(Nk,Cb,YZx),(Nk,Cb,YZy),(Nk,Cb,YZz),(Nk,Cb,YZaa),(Nk,Cb,YZab),(Nk,C b,YZac),(Nk,Cb,YZad),(Nk,Cb,YZae),(Nk,Cb,YZaf),(Nk,Cb,YZag),(Nk,Cc,YZa),(Nk,Cc,Y Zb),(Nk,Cc,YZc),(Nk,Cc,YZd),(Nk,Cc,YZe),(Nk,Cc,YZf),(Nk,Cc,YZg),(Nk,Cc,YZh),(Nk,C c,YZi),(Nk,Cc,YZj),(Nk,Cc,YZk),(Nk,Cc,YZl),(Nk,Cc,YZm),(Nk,Cc,YZn),(Nk,Cc,YZo),(N k,Cc,YZp),(Nk,Cc,YZq),(Nk,Cc,YZr),(Nk,Cc,YZs),(Nk,Cc,YZt),(Nk,Cc,YZu),(Nk,Cc,YZv), (Nk,Cc,YZw),(Nk,Cc,YZx),(Nk,Cc,YZy),(Nk,Cc,YZz),(Nk,Cc,YZaa),(Nk,Cc,YZab),(Nk,Cc ,YZac),(Nk,Cc,YZad),(Nk,Cc,YZae),(Nk,Cc,YZaf),(Nk,Cc,YZag),(Nk,Cd,YZa),(Nk,Cd,YZb) ,(Nk,Cd,YZc),(Nk,Cd,YZd),(Nk,Cd,YZe),(Nk,Cd,YZf),(Nk,Cd,YZg),(Nk,Cd,YZh),(Nk,Cd, YZi),(Nk,Cd,YZj),(Nk,Cd,Ya),(Nk,Cd,YZl),(Nk,Cd,YZm),(Nk,Cd,YZn),(Nk,Cd,YZo),(Nk ,Cd,YZp),(Nk,Cd,YZq),(Nk,Cd,YZr),(Nk,Cd,YZs),(Nk,Cd,YZt),(Nk,Cd,YZu),(Nk,Cd,YZv), (Nk,Cd,YZw),(Nk,Cd,YZx),(Nk,Cd,YZy),(Nk,Cd,YZz),(Nk,Cd,YZaa),(Nk,Cd,YZab),(Nk,C d,YZac),(Nk,Cd,YZad),(Nk,Cd,YZae),(Nk,Cd,YZaf),(Nk,Cd,YZag),(Nk,Ce,YZa),(Nk,Ce,Y Zb),(Nk,Ce,YZc),(Nk,Ce,YZd),(Nk,Ce,YZe),(Nk,Ce,YZf),(Nk,Ce,YZg),(Nk,Ce,YZh),(Nk,C e,YZi),(Nk,Ce,YZj),(Nk,Ce,YZk),(Nk,Ce,YZl),(Nk,Ce,YZm),(Nk,Ce,YZn),(Nk,Ce,YZo),(N k,Ce,YZp),(Nk,Ce,YZq),(Nk,Ce,YZr),(Nk,Ce,YZs),(Nk,Ce,YZt),(Nk,Ce,YZu),(Nk,Ce,YZv), (Nk,Ce,YZw),(Nk,Ce,YZx),(Nk,Ce,YZy),(Nk,Ce,YZz),(Nk,Ce,YZaa),(Nk,Ce,YZab),(Nk,Ce ,YZac),(Nk,Ce,YZad),(Nk,Ce,YZae),(Nk,Ce,YZaf),(Nk,Ce,YZag),(Nk,Cf,YZa),(Nk,Cf,YZb) ,(Nk,Cf,YZc),(Nk,Cf,YZd),(Nk,Cf,YZe),(Nk,Cf,YZf),(Nk,Cf,YZg),(Nk,Cf,YZh),(Nk,Cf,YZi) ,(Nk,Cf,YZj),(Nk,Cf,YZk),(Nk,Cf,YZl),(Nk,Cf,YZm),(Nk,Cf,YZn),(Nk,Cf,YZo),(Nk,Cf,Y Zp),(Nk,Cf,YZq),(Nk,Cf,YZr),(Nk,Cf,YZs),(Nk,Cf,YZt),(Nk,Cf,YZu),(Nk,Cf,YZv),(Nk,Cf, YZw),(Nk,Cf,YZx),(Nk,Cf,YZy),(Nk,Cf,YZz),(Nk,Cf,YZaa),(Nk,Cf,YZab),(Nk,Cf,YZac),( Nk,Cf,YZad),(Nk,Cf,YZae),(Nk,Cf,YZaf),(Nk,Cf,YZag),(Nk,Cg,YZa),(Nk,Cg,YZb),(Nk,Cg, YZc),(Nk,Cg,YZd),(Nk,Cg,YZe),(Nk,Cg,YZf),(Nk,Cg,YZg),(Nk,Cg,YZh),(Nk,Cg,YZi),(Nk, Cg,YZj),(Nk,Cg,YZk),(Nk,Cg,YZl),(Nk,Cg,YZm),(Nk,Cg,YZn),(Nk,Cg,YZo),(Nk,Cg,YZp), (Nk,Cg,YZq),(Nk,Cg,YZr),(Nk,Cg,YZs),(Nk,Cg,YZt),(Nk,Cg,YZu),(Nk,Cg,YZv),(Nk,Cg,Y Zw),(Nk,Cg,YZx),(Nk,Cg,YZy),(Nk,Cg,YZz),(Nk,Cg,YZaa),(Nk,Cg,YZab),(Nk,Cg,YZac),( Nk,Cg,YZad),(Nk,Cg,YZae),(Nk,Cg,YZaf),(Nk,Cg,YZag),(Nk,Ch,YZa),(Nk,Ch,YZb),(Nk,C h,YZc),(Nk,Ch,YZd),(Nk,Ch,YZe),(Nk,Ch,YZf),(Nk,Ch,YZg),(Nk,Ch,YZh),(Nk,Ch,YZi),(N k,Ch,YZj),(Nk,Ch,YZk),(Nk,Ch,YZl),(Nk,Ch,YZm),(Nk,Ch,YZn),(Nk,Ch,YZo),(Nk,Ch,YZ p),(Nk,Ch,YZq),(Nk,Ch,YZr),(Nk,Ch,YZs),(Nk,Ch,YZt),(Nk,Ch,YZu),(Nk,Ch,YZv),(Nk,Ch ,YZw),(Nk,Ch,YZx),(Nk,Ch,YZy),(Nk,Ch,YZz),(Nk,Ch,YZaa),(Nk,Ch,YZab),(Nk,Ch,YZac) ,(Nk,Ch,YZad),(Nk,Ch,YZae),(Nk,Ch,YZaf),(Nk,Ch,YZag),(Nk,Ci,YZa),(Nk,Ci,YZb),(Nk, Ci,YZc),(Nk,Ci,YZd),(Nk,Ci,YZe),(Nk,Ci,YZf),(Nk,Ci,YZg),(Nk,Ci,YZh),(Nk,Ci,YZi),(Nk, Ci,YZj),(Nk,Ci,YZk),(Nk,Ci,YZl),(Nk,Ci,YZm),(Nk,Ci,YZn),(Nk,Ci,YZo),(Nk,Ci,YZp),(Nk ,Ci,YZq),(Nk,Ci,YZr),(Nk,Ci,YZs),(Nk,Ci,YZt),(Nk,Ci,YZu),(Nk,Ci,YZv),(Nk,Ci,YZw),(N k,Ci,YZx),(Nk,Ci,YZy),(Nk,Ci,YZz),(Nk,Ci,YZaa),(Nk,Ci,YZab),(Nk,Ci,YZac),(Nk,Ci,YZa d),(Nk,Ci,YZae),(Nk,Ci,YZaf),(Nk,Ci,YZag),(Nk,Cj,YZa),(Nk,Cj,YZb),(Nk,Cj,YZc),(Nk,Cj, YZd),(Nk,Cj,YZe),(Nk,Cj,YZf),(Nk,Cj,YZg),(Nk,Cj,YZh),(Nk,Cj,YZi),(Nk,Cj,YZj),(Nk,Cj, YZk),(Nk,Cj,YZl),(Nk,Cj,YZm),(Nk,Cj,YZn),(Nk,Cj,YZo),(Nk,Cj,YZp),(Nk,Cj,YZq),(Nk,C j,YZr),(Nk,Cj,YZs),(Nk,Cj,YZt),(Nk,Cj,YZu),(Nk,Cj,YZv),(Nk,Cj,YZw),(Nk,Cj,YZx),(Nk, Cj,YZy),(Nk,Cj,YZz),(Nk,Cj,YZaa),(Nk,Cj,YZab),(Nk,Cj,YZac),(Nk,Cj,YZad),(Nk,Cj,YZae) ,(Nk,Cj,YZaf),(Nk,Cj,YZag),(Nl,Ca,YZa),(Nl,Ca,YZb),(Nl,Ca,YZc),(Nl,Ca,YZd),(Nl,Ca,Y Ze),(Nl,Ca,YZf),(Nl,Ca,YZg),(Nl,Ca,YZh),(Nl,Ca,YZi),(Nl,Ca,YZj),(Nl,Ca,YZk),(Nl,Ca,YZ l),(Nl,Ca,YZm),(Nl,Ca,YZn),(Nl,Ca,YZo),(Nl,Ca,YZp),(Nl,Ca,YZq),(Nl,Ca,YZr),(Nl,Ca,YZ s),(Nl,Ca,YZt),(Nl,Ca,YZu),(Nl,Ca,YZv),(Nl,Ca,YZw),(Nl,Ca,YZx),(Nl,Ca,YZy),(Nl,Ca,YZ z),(Nl,Ca,YZaa),(Nl,Ca,YZab),(Nl,Ca,YZac),(Nl,Ca,YZad),(Nl,Ca,YZae),(Nl,Ca,YZaf),(Nl, Ca,YZag),(Nl,Cb,YZa),(Nl,Cb,YZb),(Nl,Cb,YZc),(Nl,Cb,YZd),(Nl,Cb,YZe),(Nl,Cb,YZf),(N l,Cb,YZg),(Nl,Cb,YZh),(Nl,Cb,YZi),(Nl,Cb,YZj),(Nl,Cb,YZl),(Nl,Cb,YZl),(Nl,Cb,YZm),(N l,Cb,YZn),(Nl,Cb,YZo),(Nl,Cb,YZp),(Nl,Cb,YZq),(Nl,Cb,YZr),(Nl,Cb,YZs),(Nl,Cb,YZt),(N l,Cb,YZu),(Nl,Cb,YZv),(Nl,Cb,YZw),(Nl,Cb,YZx),(Nl,Cb,YZy),(Nl,Cb,YZz),(Nl,Cb,YZaa), (Nl,Cb,YZab),(Nl,Cb,YZac),(Nl,Cb,YZad),(Nl,Cb,YZae),(Nl,Cb,YZaf),(Nl,Cb,YZag),(Nl,Cc ,YZa),(Nl,Cc,YZb),(Nl,Cc,YZc),(Nl,Cc,YZd),(Nl,Cc,YZe),(Nl,Cc,YZf),(Nl,Cc,YZg),(Nl,Cc, YZh),(Nl,Cc,YZi),(Nl,Cc,YZj),(Nl,Cc,YZk),(Nl,Cc,YZl),(Nl,Cc,YZm),(Nl,Cc,YZn),(Nl,Cc, YZo),(Nl,Cc,YZp),(Nl,Cc,YZq),(Nl,Cc,YZr),(Nl,Cc,YZs),(Nl,Cc,YZt),(Nl,Cc,YZu),(Nl,Cc, YZv),(Nl,Cc,YZw),(Nl,Cc,YZx),(Nl,Cc,YZy),(Nl,Cc,YZz),(Nl,Cc,YZaa),(Nl,Cc,YZab),(Nl, Cc,YZac),(Nl,Cc,YZad),(Nl,Cc,YZae),(Nl,Cc,YZaf),(Nl,Cc,YZag),(Nl,Cd,YZa),(Nl,Cd,YZb) ,(Nl,Cd,YZc),(Nl,Cd,YZd),(Nl,Cd,YZe),(Nl,Cd,YZf),(Nl,Cd,YZg),(Nl,Cd,YZh),(Nl,Cd,YZi) ,(Nl,Cd,YZj),(Nl,Cd,YZk),(Nl,Cd,YZl),(Nl,Cd,YZm),(Nl,Cd,YZn),(Nl,Cd,YZo),(Nl,Cd,YZ p),(Nl,Cd,YZq),(Nl,Cd,YZr),(Nl,Cd,YZs),(Nl,Cd,YZt),(Nl,Cd,YZu),(Nl,Cd,YZv),(Nl,Cd,YZ w),(Nl,Cd,YZx),(Nl,Cd,YZy),(Nl,Cd,YZz),(Nl,Cd,YZaa),(Nl,Cd,YZab),(Nl,Cd,YZac),(Nl,C d,YZad),(Nl,Cd,YZae),(Nl,Cd,YZaf),(Nl,Cd,YZag),(Nl,Ce,YZa),(Nl,Ce,YZb),(Nl,Ce,YZc),( Nl,Ce,YZd),(Nl,Ce,YZe),(Nl,Ce,YZf),(Nl,Ce,YZg),(Nl,Ce,YZh),(Nl,Ce,YZi),(Nl,Ce,YZj),(N l,Ce,YZk),(Nl,Ce,YZl),(Nl,Ce,YZm),(Nl,Ce,YZn),(Nl,Ce,YZo),(Nl,Ce,YZp),(Nl,Ce,YZq),(N l,Ce,YZr),(Nl,Ce,YZs),(Nl,Ce,YZt),(Nl,Ce,YZu),(Nl,Ce,YZv),(Nl,Ce,YZw),(Nl,Ce,YZx),(Nl ,Ce,YZy),(Nl,Ce,YZz),(Nl,Ce,YZaa),(Nl,Ce,YZab),(Nl,Ce,YZac),(Nl,Ce,YZad),(Nl,Ce,YZae) ,(Nl,Ce,YZaf),(Nl,Ce,YZag),(Nl,Cf,YZa),(Nl,Cf,YZb),(Nl,Cf,YZc),(Nl,Cf,YZd),(Nl,Cf,YZe) ,(Nl,Cf,YZf),(Nl,Cf,YZg),(Nl,Cf,YZh),(Nl,Cf,YZi),(Nl,Cf,YZj),(Nl,Cf,YZk),(Nl,Cf,YZl),(N l,Cf,YZm),(Nl,Cf,YZn),(Nl,Cf,YZo),(Nl,Cf,YZp),(Nl,Cf,YZq),(Nl,Cf,YZr),(Nl,Cf,YZs),(Nl, Cf,YZt),(Nl,Cf,YZu),(Nl,Cf,YZv),(Nl,Cf,YZw),(Nl,Cf,YZx),(Nl,Cf,YZy),(Nl,Cf,YZz),(Nl,C f,YZaa),(Nl,Cf,YZab),(Nl,Cf,YZac),(Nl,Cf,YZad),(Nl,Cf,YZae),(Nl,Cf,YZaf),(Nl,Cf,YZag),( Nl,Cg,YZa),(Nl,Cg,YZb),(Nl,Cg,YZc),(Nl,Cg,YZd),(Nl,Cg,YZe),(Nl,Cg,YZf),(Nl,Cg,YZg),( Nl,Cg,YZh),(Nl,Cg,YZi),(Nl,Cg,YZj),(Nl,Cg,YZk),(Nl,Cg,YZl),(Nl,Cg,YZm),(Nl,Cg,YZn),( Nl,Cg,YZo),(Nl,Cg,YZp),(Nl,Cg,YZq),(Nl,Cg,YZr),(Nl,Cg,YZs),(Nl,Cg,YZt),(Nl,Cg,YZu),( Nl,Cg,YZv),(Nl,Cg,YZw),(Nl,Cg,YZx),(Nl,Cg,YZy),(Nl,Cg,YZz),(Nl,Cg,YZaa),(Nl,Cg,YZa b),(Nl,Cg,YZac),(Nl,Cg,YZad),(Nl,Cg,YZae),(Nl,Cg,YZaf),(Nl,Cg,YZag),(Nl,Ch,YZa),(Nl, Ch,YZb),(Nl,Ch,YZc),(Nl,Ch,YZd),(Nl,Ch,YZe),(Nl,Ch,YZf),(Nl,Ch,YZg),(Nl,Ch,YZh),(Nl ,Ch,YZi),(Nl,Ch,YZj),(Nl,Ch,YZk),(Nl,Ch,YZl),(Nl,Ch,YZm),(Nl,Ch,YZn),(Nl,Ch,YZo),(Nl ,Ch,YZp),(Nl,Ch,YZq),(Nl,Ch,YZr),(Nl,Ch,YZs),(Nl,Ch,YZt),(Nl,Ch,YZu),(Nl,Ch,YZv),(Nl ,Ch,YZw),(Nl,Ch,YZx),(Nl,Ch,YZy),(Nl,Ch,YZz),(Nl,Ch,YZaa),(Nl,Ch,YZab),(Nl,Ch,YZac) ,(Nl,Ch,YZad),(Nl,Ch,YZae),(Nl,Ch,YZaf),(Nl,Ch,YZag),(Nl,Ci,YZa),(Nl,Ci,YZb),(Nl,Ci, YZc),(Nl,Ci,YZd),(Nl,Ci,YZe),(Nl,Ci,YZf),(Nl,Ci,YZg),(Nl,Ci,YZh),(Nl,Ci,YZi),(Nl,Ci,YZj) ,(Nl,Ci,YZk),(Nl,Ci,YZl),(Nl,Ci,YZm),(Nl,Ci,YZn),(Nl,Ci,YZo),(Nl,Ci,YZp),(Nl,Ci,YZq),( Nl,Ci,YZr),(Nl,Ci,YZs),(Nl,Ci,YZt),(Nl,Ci,YZu),(Nl,Ci,YZv),(Nl,Ci,YZw),(Nl,Ci,YZx),(Nl, Ci,YZy),(Nl,Ci,YZz),(Nl,Ci,YZaa),(Nl,Ci,YZab),(Nl,Ci,YZac),(Nl,Ci,YZad),(Nl,Ci,YZae),( Nl,Ci,YZaf),(Nl,Ci,YZag),(Nl,Cj,YZa),(Nl,Cj,YZb),(Nl,Cj,YZc),(Nl,Cj,YZd),(Nl,Cj,YZe),( Nl,Cj,YZf),(Nl,Cj,YZg),(Nl,Cj,YZh),(Nl,Cj,YZi),(Nl,Cj,YZj),(Nl,Cj,YZk),(Nl,Cj,YZl),(Nl,C j,YZm),(Nl,Cj,YZn),(Nl,Cj,YZo),(Nl,Cj,YZp),(Nl,Cj,YZq),(Nl,Cj,YZr),(Nl,Cj,YZs),(Nl,Cj,Y Zt),(Nl,Cj,YZu),(Nl,Cj,YZv),(Nl,Cj,YZw),(Nl,Cj,YZx),(Nl,Cj,YZy),(Nl,Cj,YZz),(Nl,Cj,YZa a),(Nl,Cj,YZab),(Nl,Cj,YZac),(Nl,Cj,YZad),(Nl,Cj,YZae),(Nl,Cj,YZaf),(Nl,Cj,YZag),(Nm,C a,YZa),(Nm,Ca,YZb),(Nm,Ca,YZc),(Nm,Ca,YZd),(Nm,Ca,YZe),(Nm,Ca,YZf),(Nm,Ca,YZg) ,(Nm,Ca,YZh),(Nm,Ca,YZi),(Nm,Ca,YZj),(Nm,Ca,YZk),(Nm,Ca,YZl),(Nm,Ca,YZm),(Nm, Ca,YZn),(Nm,Ca,YZo),(Nm,Ca,YZp),(Nm,Ca,YZq),(Nm,Ca,YZr),(Nm,Ca,YZs),(Nm,Ca,YZ t),(Nm,Ca,YZu),(Nm,Ca,YZv),(Nm,Ca,YZw),(Nm,Ca,YZx),(Nm,Ca,YZy),(Nm,Ca,YZz),(N m,Ca,YZaa),(Nm,Ca,YZab),(Nm,Ca,YZac),(Nm,Ca,YZad),(Nm,Ca,YZae),(Nm,Ca,YZaf),(N m,Ca,YZag),(Nm,Cb,YZa),(Nm,Cb,YZb),(Nm,Cb,YZc),(Nm,Cb,YZd),(Nm,Cb,YZe),(Nm,C b,YZf),(Nm,Cb,YZg),(Nm,Cb,YZh),(Nm,Cb,YZi),(Nm,Cb,YZj),(Nm,Cb,YZk),(Nm,Cb,YZl) ,(Nm,Cb,YZm),(Nm,Cb,YZn),(Nm,Cb,YZo),(Nm,Cb,YZp),(Nm,Cb,YZq),(Nm,Cb,YZr),(N m,Cb,YZs),(Nm,Cb,YZt),(Nm,Cb,YZu),(Nm,Cb,YZv),(Nm,Cb,YZw),(Nm,Cb,YZx),(Nm,Cb ,YZy),(Nm,Cb,YZz),(Nm,Cb,YZaa),(Nm,Cb,YZab),(Nm,Cb,YZac),(Nm,Cb,YZad),(Nm,Cb, YZae),(Nm,Cb,YZaf),(Nm,Cb,YZag),(Nm,Cc,YZa),(Nm,Cc,YZb),(Nm,Cc,YZc),(Nm,Cc,YZ d),(Nm,Cc,YZe),(Nm,Cc,YZf),(Nm,Cc,YZg),(Nm,Cc,YZl),(Nm,Cc,YZi),(Nm,Cc,YZj),(Nm ,Cc,YZk),(Nm,Cc,YZl),(Nm,Cc,YZm),(Nm,Cc,YZn),(Nm,Cc,YZo),(Nm,Cc,YZp),(Nm,Cc,Y Zq),(Nm,Cc,YZr),(Nm,Cc,YZs),(Nm,Cc,YZt),(Nm,Cc,YZu),(Nm,Cc,YZv),(Nm,Cc,YZw),( Nm,Cc,YZx),(Nm,Cc,YZy),(Nm,Cc,YZz),(Nm,Cc,YZaa),(Nm,Cc,YZab),(Nm,Cc,YZac),(N m,Cc,YZad),(Nm,Cc,YZae),(Nm,Cc,YZaf),(Nm,Cc,YZag),(Nm,Cd,YZa),(Nm,Cd,YZb),(Nm ,Cd,YZc),(Nm,Cd,YZd),(Nm,Cd,YZe),(Nm,Cd,YZf),(Nm,Cd,YZg),(Nm,Cd,YZh),(Nm,Cd,Y Zi),(Nm,Cd,YZj),(Nm,Cd,YZk),(Nm,Cd,YZl),(Nm,Cd,YZm),(Nm,Cd,YZn),(Nm,Cd,YZo),( Nm,Cd,YZp),(Nm,Cd,YZq),(Nm,Cd,YZr),(Nm,Cd,YZs),(Nm,Cd,YZt),(Nm,Cd,YZu),(Nm,C d,YZv),(Nm,Cd,YZw),(Nm,Cd,YZx),(Nm,Cd,YZy),(Nm,Cd,YZz),(Nm,Cd,YZaa),(Nm,Cd,Y Zab),(Nm,Cd,YZac),(Nm,Cd,YZad),(Nm,Cd,YZae),(Nm,Cd,YZaf),(Nm,Cd,YZag),(Nm,Ce, YZa),(Nm,Ce,YZb),(Nm,Ce,YZc),(Nm,Ce,YZd),(Nm,Ce,YZe),(Nm,Ce,YZf),(Nm,Ce,YZg),( Nm,Ce,YZh),(Nm,Ce,YZi),(Nm,Ce,YZj),(Nm,Ce,YZk),(Nm,Ce,YZl),(Nm,Ce,YZm),(Nm,C e,YZn),(Nm,Ce,YZo),(Nm,Ce,YZp),(Nm,Ce,YZq),(Nm,Ce,YZr),(Nm,Ce,YZs),(Nm,Ce,YZt) ,(Nm,Ce,YZu),(Nm,Ce,YZv),(Nm,Ce,YZw),(Nm,Ce,YZx),(Nm,Ce,YZy),(Nm,Ce,YZz),(Nm ,Ce,YZaa),(Nm,Ce,YZab),(Nm,Ce,YZac),(Nm,Ce,YZad),(Nm,Ce,YZae),(Nm,Ce,YZaf),(Nm ,Ce,YZag),(Nm,Cf,YZa),(Nm,Cf,YZb),(Nm,Cf,YZc),(Nm,Cf,YZd),(Nm,Cf,YZe),(Nm,Cf,Y Zf),(Nm,Cf,YZg),(Nm,Cf,YZh),(Nm,Cf,YZi),(Nm,Cf,YZj),(Nm,Cf,YZk),(Nm,Cf,YZl),(Nm, Cf,YZm),(Nm,Cf,YZn),(Nm,Cf,YZo),(Nm,Cf,YZp),(Nm,Cf,YZq),(Nm,Cf,YZr),(Nm,Cf,YZs) ,(Nm,Cf,YZt),(Nm,Cf,YZu),(Nm,Cf,YZv),(Nm,Cf,YZw),(Nm,Cf,YZx),(Nm,Cf,YZy),(Nm, Cf,YZz),(Nm,Cf,YZaa),(Nm,Cf,YZab),(Nm,Cf,YZac),(Nm,Cf,YZad),(Nm,Cf,YZae),(Nm,Cf ,YZaf),(Nm,Cf,YZag),(Nm,Cg,YZa),(Nm,Cg,YZb),(Nm,Cg,YZc),(Nm,Cg,YZd),(Nm,Cg,YZ e),(Nm,Cg,YZf),(Nm,Cg,YZg),(Nm,Cg,YZh),(Nm,Cg,YZi),(Nm,Cg,YZj),(Nm,Cg,YZk),(N m,Cg,YZl),(Nm,Cg,YZm),(Nm,Cg,YZn),(Nm,Cg,YZo),(Nm,Cg,YZp),(Nm,Cg,YZq),(Nm,C g,YZr),(Nm,Cg,YZs),(Nm,Cg,YZt),(Nm,Cg,YZu),(Nm,Cg,YZv),(Nm,Cg,YZw),(Nm,Cg,YZ x),(Nm,Cg,YZy),(Nm,Cg,YZz),(Nm,Cg,YZaa),(Nm,Cg,YZab),(Nm,Cg,YZac),(Nm,Cg,YZad) ,(Nm,Cg,YZae),(Nm,Cg,YZaf),(Nm,Cg,YZag),(Nm,Ch,YZa),(Nm,Ch,YZb),(Nm,Ch,YZc),( Nm,Ch,YZd),(Nm,Ch,YZe),(Nm,Ch,YZf),(Nm,Ch,YZg),(Nm,Ch,YZh),(Nm,Ch,YZi),(Nm,C h,YZj),(Nm,Ch,YZk),(Nm,Ch,YZl),(Nm,Ch,YZm),(Nm,Ch,YZn),(Nm,Ch,YZo),(Nm,Ch,YZ p),(Nm,Ch,YZq),(Nm,Ch,YZr),(Nm,Ch,YZs),(Nm,Ch,YZt),(Nm,Ch,YZu),(Nm,Ch,YZv),(N m,Ch,YZw),(Nm,Ch,YZx),(Nm,Ch,YZy),(Nm,Ch,YZz),(Nm,Ch,YZaa),(Nm,Ch,YZab),(Nm, Ch,YZac),(Nm,Ch,YZad),(Nm,Ch,YZae),(Nm,Ch,YZaf),(Nm,Ch,YZag),(Nm,Ci,YZa),(Nm, Ci,YZb),(Nm,Ci,YZc),(Nm,Ci,YZd),(Nm,Ci,YZe),(Nm,Ci,YZf),(Nm,Ci,YZg),(Nm,Ci,YZh), (Nm,Ci,YZi),(Nm,Ci,YZj),(Nm,Ci,YZk),(Nm,Ci,YZl),(Nm,Ci,YZm),(Nm,Ci,YZn),(Nm,Ci, YZo),(Nm,Ci,YZp),(Nm,Ci,YZq),(Nm,Ci,YZr),(Nm,Ci,YZs),(Nm,Ci,YZt),(Nm,Ci,YZu),(N m,Ci,YZv),(Nm,Ci,YZw),(Nm,Ci,YZx),(Nm,Ci,YZy),(Nm,Ci,YZz),(Nm,Ci,YZaa),(Nm,Ci, YZab),(Nm,Ci,YZac),(Nm,Ci,YZad),(Nm,Ci,YZae),(Nm,Ci,YZaf),(Nm,Ci,YZag),(Nm,Cj,Y Za),(Nm,Cj,YZb),(Nm,Cj,YZc),(Nm,Cj,YZd),(Nm,Cj,YZe),(Nm,Cj,YZf),(Nm,Cj,YZg),(Nm, Cj,YZh),(Nm,Cj,YZi),(Nm,Cj,YZj),(Nm,Cj,YZk),(Nm,Cj,YZl),(Nm,Cj,YZm),(Nm,Cj,YZn), (Nm,Cj,YZo),(Nm,Cj,YZp),(Nm,Cj,YZq),(Nm,Cj,YZr),(Nm,Cj,YZs),(Nm,Cj,YZt),(Nm,Cj, YZu),(Nm,Cj,YZv),(Nm,Cj,YZw),(Nm,Cj,YZx),(Nm,Cj,YZy),(Nm,Cj,YZz),(Nm,Cj,YZaa),( Nm,Cj,YZab),(Nm,Cj,YZac),(Nm,Cj,YZad),(Nm,Cj,YZae),(Nm,Cj,YZaf),(Nm,Cj,YZag),(N n,Ca,YZa),(Nn,Ca,YZb),(Nn,Ca,YZc),(Nn,Ca,YZd),(Nn,Ca,YZe),(Nn,Ca,YZf),(Nn,Ca,YZg) ,(Nn,Ca,YZh),(Nn,Ca,YZi),(Nn,Ca,YZj),(Nn,Ca,YZk),(Nn,Ca,YZl),(Nn,Ca,YZm),(Nn,Ca,Y Zn),(Nn,Ca,YZo),(Nn,Ca,YZp),(Nn,Ca,YZq),(Nn,Ca,YZr),(Nn,Ca,YZs),(Nn,Ca,YZt),(Nn,Ca ,YZu),(Nn,Ca,YZv),(Nn,Ca,YZw),(Nn,Ca,YZx),(Nn,Ca,YZy),(Nn,Ca,YZz),(Nn,Ca,YZaa),( Nn,Ca,YZab),(Nn,Ca,YZac),(Nn,Ca,YZad),(Nn,Ca,YZae),(Nn,Ca,YZaf),(Nn,Ca,YZag),(Nn, Cb,YZa),(Nn,Cb,YZb),(Nn,Cb,YZc),(Nn,Cb,YZd),(Nn,Cb,YZe),(Nn,Cb,YZf),(Nn,Cb,YZg), (Nn,Cb,YZh),(Nn,Cb,YZi),(Nn,Cb,YZj),(Nn,Cb,YZk),(Nn,Cb,YZl),(Nn,Cb,YZm),(Nn,Cb,Y Zn),(Nn,Cb,YZo),(Nn,Cb,YZp),(Nn,Cb,YZq),(Nn,Cb,YZr),(Nn,Cb,YZs),(Nn,Cb,YZt),(Nn,C b,YZu),(Nn,Cb,YZv),(Nn,Cb,YZw),(Nn,Cb,YZx),(Nn,Cb,YZy),(Nn,Cb,YZz),(Nn,Cb,YZaa), (Nn,Cb,YZab),(Nn,Cb,YZac),(Nn,Cb,YZad),(Nn,Cb,YZae),(Nn,Cb,YZaf),(Nn,Cb,YZag),(N n,Cc,YZa),(Nn,Cc,YZb),(Nn,Cc,YZc),(Nn,Cc,YZd),(Nn,Cc,YZe),(Nn,Cc,YZf),(Nn,Cc,YZg) ,(Nn,Cc,YZh),(Nn,Cc,YZi),(Nn,Cc,YZj),(Nn,Cc,YZk),(Nn,Cc,YZl),(Nn,Cc,YZm),(Nn,Cc,Y Zn),(Nn,Cc,YZo),(Nn,Cc,YZp),(Nn,Cc,YZq),(Nn,Cc,YZr),(Nn,Cc,YZs),(Nn,Cc,YZt),(Nn,Cc ,YZu),(Nn,Cc,YZv),(Nn,Cc,YZw),(Nn,Cc,YZx),(Nn,Cc,YZy),(Nn,Cc,YZz),(Nn,Cc,YZaa),( Nn,Cc,YZab),(Nn,Cc,YZac),(Nn,Cc,YZad),(Nn,Cc,YZae),(Nn,Cc,YZaf),(Nn,Cc,YZag),(Nn, Cd,YZa),(Nn,Cd,YZb),(Nn,Cd,YZc),(Nn,Cd,YZd),(Nn,Cd,YZe),(Nn,Cd,YZf),(Nn,Cd,YZg), (Nn,Cd,YZh),(Nn,Cd,YZi),(Nn,Cd,YZj),(Nn,Cd,YZk),(Nn,Cd,YZl),(Nn,Cd,YZm),(Nn,Cd,Y Zn),(Nn,Cd,YZo),(Nn,Cd,YZp),(Nn,Cd,YZq),(Nn,Cd,YZr),(Nn,Cd,YZs),(Nn,Cd,YZt),(Nn,C d,YZu),(Nn,Cd,YZv),(Nn,Cd,YZw),(Nn,Cd,YZx),(Nn,Cd,YZy),(Nn,Cd,YZz),(Nn,Cd,YZaa), (Nn,Cd,YZab),(Nn,Cd,YZac),(Nn,Cd,YZad),(Nn,Cd,YZae),(Nn,Cd,YZaf),(Nn,Cd,YZag),(N n,Ce,YZa),(Nn,Ce,YZb),(Nn,Ce,YZc),(Nn,Ce,YZd),(Nn,Ce,YZe),(Nn,Ce,YZf),(Nn,Ce,YZg) ,(Nn,Ce,YZh),(Nn,Ce,YZi),(Nn,Ce,YZj),(Nn,Ce,YZk),(Nn,Ce,YZl),(Nn,Ce,YZm),(Nn,Ce,Y Zn),(Nn,Ce,YZo),(Nn,Ce,YZp),(Nn,Ce,YZq),(Nn,Ce,YZr),(Nn,Ce,YZs),(Nn,Ce,YZt),(Nn,Ce ,YZu),(Nn,Ce,YZv),(Nn,Ce,YZw),(Nn,Ce,YZx),(Nn,Ce,YZy),(Nn,Ce,YZz),(Nn,Ce,YZaa),( Nn,Ce,YZab),(Nn,Ce,YZac),(Nn,Ce,YZad),(Nn,Ce,YZae),(Nn,Ce,YZaf),(Nn,Ce,YZag),(Nn, Cf,YZa),(Nn,Cf,YZb),(Nn,Cf,YZc),(Nn,Cf,YZd),(Nn,Cf,YZe),(Nn,Cf,YZf),(Nn,Cf,YZg),(N n,Cf,YZh),(Nn,Cf,YZi),(Nn,Cf,YZj),(Nn,Cf,YZk),(Nn,Cf,YZl),(Nn,Cf,YZm),(Nn,Cf,YZn),( Nn,Cf,YZo),(Nn,Cf,YZp),(Nn,Cf,YZq),(Nn,Cf,YZr),(Nn,Cf,YZs),(Nn,Cf,YZt),(Nn,Cf,YZu), (Nn,Cf,YZv),(Nn,Cf,YZw),(Nn,Cf,YZx),(Nn,Cf,YZy),(Nn,Cf,YZz),(Nn,Cf,YZaa),(Nn,Cf,Y Zab),(Nn,Cf,YZac),(Nn,Cf,YZad),(Nn,Cf,YZae),(Nn,Cf,YZaf),(Nn,Cf,YZag),(Nn,Cg,YZa),( Nn,Cg,YZb),(Nn,Cg,YZc),(Nn,Cg,YZd),(Nn,Cg,YZe),(Nn,Cg,YZf),(Nn,Cg,YZg),(Nn,Cg,Y Zh),(Nn,Cg,YZi),(Nn,Cg,YZj),(Nn,Cg,YZk),(Nn,Cg,YZl),(Nn,Cg,YZm),(Nn,Cg,YZn),(Nn,C g,YZo),(Nn,Cg,YZp),(Nn,Cg,YZq),(Nn,Cg,YZr),(Nn,Cg,YZs),(Nn,Cg,YZt),(Nn,Cg,YZu),(N n,Cg,YZv),(Nn,Cg,YZw),(Nn,Cg,YZx),(Nn,Cg,YZy),(Nn,Cg,YZz),(Nn,Cg,YZaa),(Nn,Cg,Y Zab),(Nn,Cg,YZac),(Nn,Cg,YZad),(Nn,Cg,YZae),(Nn,Cg,YZaf),(Nn,Cg,YZag),(Nn,Ch,YZa) ,(Nn,Ch,YZb),(Nn,Ch,YZc),(Nn,Ch,YZd),(Nn,Ch,YZe),(Nn,Ch,YZf),(Nn,Ch,YZg),(Nn,Ch, YZh),(Nn,Ch,YZi),(Nn,Ch,YZj),(Nn,Ch,YZk),(Nn,Ch,YZl),(Nn,Ch,YZm),(Nn,Ch,YZn),(Nn ,Ch,YZo),(Nn,Ch,YZp),(Nn,Ch,YZq),(Nn,Ch,YZr),(Nn,Ch,YZs),(Nn,Ch,YZt),(Nn,Ch,YZu), (Nn,Ch,YZv),(Nn,Ch,YZw),(Nn,Ch,YZx),(Nn,Ch,YZy),(Nn,Ch,YZz),(Nn,Ch,YZaa),(Nn,Ch, YZab),(Nn,Ch,YZac),(Nn,Ch,YZad),(Nn,Ch,YZae),(Nn,Ch,YZaf),(Nn,Ch,YZag),(Nn,Ci,YZ a),(Nn,C i,YZb),(Nn,Ci,YZc),(Nn,Ci,YZd),(Nn,Ci,YZe),(Nn,Ci,YZf),(Nn,Ci,YZg),(Nn,Ci,YZ h),(Nn,Ci,YZi),(Nn,Ci,YZj),(Nn,Ci,YZk),(Nn,Ci,YZl),(Nn,Ci,YZm),(Nn,Ci,YZn),(Nn,Ci,YZ o),(Nn,Ci,YZp),(Nn,Ci,YZq),(Nn,Ci,YZr),(Nn,Ci,YZs),(Nn,Ci,YZt),(Nn,Ci,YZu),(Nn,Ci,YZ v),(Nn,Ci,YZw),(Nn,Ci,YZx),(Nn,Ci,YZy),(Nn,Ci,YZz),(Nn,Ci,YZaa),(Nn,Ci,YZab),(Nn,Ci, YZac),(Nn,Ci,YZad),(Nn,Ci,YZae),(Nn,Ci,YZaf),(Nn,Ci,YZag),(Nn,Cj,YZa),(Nn,Cj,YZb),( Nn,Cj,YZc),(Nn,Cj,YZd),(Nn,Cj,YZe),(Nn,Cj,YZf),(Nn,Cj,YZg),(Nn,Cj,YZh),(Nn,Cj,YZi),( Nn,Cj,YZj),(Nn,Cj,YZk),(Nn,Cj,YZl),(Nn,Cj,YZm),(Nn,Cj,YZn),(Nn,Cj,YZo),(Nn,Cj,YZp), (Nn,Cj,YZq),(Nn,Cj,YZr),(Nn,Cj,YZs),(Nn,Cj,YZt),(Nn,Cj,YZu),(Nn,Cj,YZv),(Nn,Cj,YZw) ,(Nn,Cj,YZx),(Nn,Cj,YZy),(Nn,Cj,YZz),(Nn,Cj,YZaa),(Nn,Cj,YZab),(Nn,Cj,YZac),(Nn,Cj, YZad),(Nn,Cj,YZae),(Nn,Cj,YZaf),(Nn,Cj,YZag),(No,Ca,YZa),(No,Ca,YZb),(No,Ca,YZc),( No,Ca,YZd),(No,Ca,YZe),(No,Ca,YZf),(No,Ca,YZg),(No,Ca,YZh),(No,Ca,YZi),(No,Ca,YZj) ,(No,Ca,YZk),(No,Ca,YZl),(No,Ca,YZm),(No,Ca,YZn),(No,Ca,YZo),(No,Ca,YZp),(No,Ca, YZq),(No,Ca,YZr),(No,Ca,YZs),(No,Ca,YZt),(No,Ca,YZu),(No,Ca,YZv),(No,Ca,YZw),(No, Ca,YZx),(No,Ca,YZy),(No,Ca,YZz),(No,Ca,YZaa),(No,Ca,YZab),(No,Ca,YZac),(No,Ca,YZ ad),(No,Ca,YZae),(No,Ca,YZaf),(No,Ca,YZag),(No,Cb,YZa),(No,Cb,YZb),(No,Cb,YZc),(N o,Cb,YZd),(No,Cb,YZe),(No,Cb,YZf),(No,Cb,YZg),(No,Cb,YZh),(No,Cb,YZi),(No,Cb,YZj) ,(No,Cb,YZk),(No,Cb,YZl),(No,Cb,YZm),(No,Cb,YZn),(No,Cb,YZo),(No,Cb,YZp),(No,Cb, YZq),(No,Cb,YZr),(No,Cb,YZs),(No,Cb,YZt),(No,Cb,YZu),(No,Cb,YZv),(No,Cb,YZw),(No ,Cb,YZx),(No,Cb,YZy),(No,Cb,YZz),(No,Cb,YZaa),(No,Cb,YZab),(No,Cb,YZac),(No,Cb,Y Zad),(No,Cb,YZae),(No,Cb,YZaf),(No,Cb,YZag),(No,Cc,YZa),(No,Cc,YZb),(No,Cc,YZc),( No,Cc,YZd),(No,Cc,YZe),(No,Cc,YZf),(No,Cc,YZg),(No,Cc,YZh),(No,Cc,YZi),(No,Cc,YZj) ,(No,Cc,YZk),(No,Cc,YZl),(No,Cc,YZm),(No,Cc,YZn),(No,Cc,YZo),(No,Cc,YZp),(No,Cc, YZq),(No,Cc,YZr),(No,Cc,YZs),(No,Cc,YZt),(No,Cc,YZu),(No,Cc,YZv),(No,Cc,YZw),(No, Cc,YZx),(No,Cc,YZy),(No,Cc,YZz),(No,Cc,YZaa),(No,Cc,YZab),(No,Cc,YZac),(No,Cc,YZ ad),(No,Cc,YZae),(No,Cc,YZaf),(No,Cc,YZag),(No,Cd,YZa),(No,Cd,YZb),(No,Cd,YZc),(N o,Cd,YZd),(No,Cd,YZe),(No,Cd,YZf),(No,Cd,YZg),(No,Cd,YZh),(No,Cd,YZi),(No,Cd,YZj) ,(No,Cd,YZk),(No,Cd,YZl),(No,Cd,YZm),(No,Cd,YZn),(No,Cd,YZo),(No,Cd,YZp),(No,Cd, YZq),(No,Cd,YZr),(No,Cd,YZs),(No,Cd,YZt),(No,Cd,YZu),(No,Cd,YZv),(No,Cd,YZw),(No ,Cd,YZx),(No,Cd,YZy),(No,Cd,YZz),(No,Cd,YZaa),(No,Cd,YZab),(No,Cd,YZac),(No,Cd,Y Zad),(No,Cd,YZae),(No,Cd,YZaf),(No,Cd,YZag),(No,Ce,YZa),(No,Ce,YZb),(No,Ce,YZc),( No,Ce,YZd),(No,Ce,YZe),(No,Ce,YZf),(No,Ce,YZg),(No,Ce,YZh),(No,Ce,YZi),(No,Ce,YZj) ,(No,Ce,YZk),(No,Ce,YZl),(No,Ce,YZm),(No,Ce,YZn),(No,Ce,YZo),(No,Ce,YZp),(No,Ce, YZq),(No,Ce,YZr),(No,Ce,YZs),(No,Ce,YZt),(No,Ce,YZu),(No,Ce,YZv),(No,Ce,YZw),(No, Ce,YZx),(No,Ce,YZy),(No,Ce,YZz),(No,Ce,YZaa),(No,Ce,YZab),(No,Ce,YZac),(No,Ce,YZ ad),(No,Ce,YZae),(No,Ce,YZaf),(No,Ce,YZag),(No,Cf,YZa),(No,Cf,YZb),(No,Cf,YZc),(No, Cf,YZd),(No,Cf,YZe),(No,Cf,YZf),(No,Cf,YZg),(No,Cf,YZh),(No,Cf,YZi),(No,Cf,YZj),(No ,Cf,YZk),(No,Cf,YZl),(No,Cf,YZm),(No,Cf,YZn),(No,Cf,YZo),(No,Cf,YZp),(No,Cf,YZq),( No,Cf,YZr),(No,Cf,YZs),(No,Cf,YZt),(No,Cf,YZu),(No,Cf,YZv),(No,Cf,YZw),(No,Cf,YZx) ,(No,Cf,YZy),(No,Cf,YZz),(No,Cf,YZaa),(No,Cf,YZab),(No,Cf,YZac),(No,Cf,YZad),(No,Cf ,YZae),(No,Cf,YZaf),(No,Cf,YZag),(No,Cg,YZa),(No,Cg,YZb),(No,Cg,YZc),(No,Cg,YZd),( No,Cg,YZe),(No,Cg,YZf),(No,Cg,YZg),(No,Cg,YZh),(No,Cg,YZi),(No,Cg,YZj),(No,Cg,YZ k),(No,Cg,YZl),(No,Cg,YZm),(No,Cg,YZn),(No,Cg,YZo),(No,Cg,YZp),(No,Cg,YZq),(No,C g,YZr),(No,Cg,YZs),(No,Cg,YZt),(No,Cg,YZu),(No,Cg,YZv),(No,Cg,YZw),(No,Cg,YZx),( No,Cg,YZy),(No,Cg,YZz),(No,Cg,YZaa),(No,Cg,YZab),(No,Cg,YZac),(No,Cg,YZad),(No,C g,YZae),(No,Cg,YZaf),(No,Cg,YZag),(No,Ch,YZa),(No,Ch,YZb),(No,Ch,YZc),(No,Ch,YZd) ,(No,Ch,YZe),(No,Ch,YZf),(No,Ch,YZg),(No,Ch,YZh),(No,Ch,YZi),(No,Ch,YZj),(No,Ch, YZk),(No,Ch,YZl),(No,Ch,YZm),(No,Ch,YZn),(No,Ch,YZo),(No,Ch,YZp),(No,Ch,YZq),(N o,Ch,YZr),(No,Ch,YZs),(No,Ch,YZt),(No,Ch,YZu),(No,Ch,YZv),(No,Ch,YZw),(No,Ch,YZx) ,(No,Ch,YZy),(No,Ch,YZz),(No,Ch,YZaa),(No,Ch,YZab),(No,Ch,YZac),(No,Ch,YZad),(No ,Ch,YZae),(No,Ch,YZaf),(No,Ch,YZag),(No,Ci,YZa),(No,Ci,YZb),(No,Ci,YZc),(No,Ci,YZd, ),(No,Ci,YZe),(No,Ci,YZf),(No,Ci,YZg),(No,Ci,YZh),(No,Ci,YZi),(No,Ci,YZj),(No,Ci,YZk) ,(No,Ci,YZl),(No,Ci,YZm),(No,Ci,YZn),(No,Ci,YZo),(No,Ci,YZp),(No,Ci,YZq),(No,Ci,YZr) ,(No,Ci,YZs),(No,Ci,YZt),(No,Ci,YZu),(No,Ci,YZy),(No,Ci,YZw),(No,Ci,YZx),(No,Ci,YZ y),(No,Ci,YZz),(No,Ci,YZaa),(No,Ci,YZab),(No,Ci,YZac),(No,Ci,YZad),(No,Ci,YZae),(No, Ci,YZaf),(No,Ci,YZag),(No,Cj,YZa),(No,Cj,YZb),(No,Cj,YZc),(No,Cj,YZd),(No,Cj,YZe),( No,Cj,YZf),(No,Cj,YZg),(No,Cj,YZh),(No,Cj,YZi),(No,Cj,YZj),(No,Cj,YZk),(No,Cj,YZl),( No,Cj,YZm),(No,Cj,YZn),(No,Cj,YZo),(No,Cj,YZp),(No,Cj,YZq),(No,Cj,YZr),(No,Cj,YZs), (No,Cj,YZt),(No,Cj,YZu),(No,Cj,YZy),(No,Cj,YZw),(No,Cj,YZx),(No,Cj,YZy),(No,Cj,YZz) ,(No,Cj,YZaa),(No,Cj,YZab),(No,Cj,YZac),(No,Cj,YZad),(No,Cj,YZae),(No,Cj,YZaf),(No,C j,YZag),(Np,Ca,YZa),(Np,Ca,YZb),(Np,Ca,YZc),(Np,Ca,YZd),(Np,Ca,YZe),(Np,Ca,YZf),(N p,Ca,YZg),(Np,Ca,YZh),(Np,Ca,YZi),(Np,Ca,YZj),(Np,Ca,YZk),(Np,Ca,YZl),(Np,Ca,YZm) ,(Np,Ca,YZn),(Np,Ca,YZo),(Np,Ca,YZp),(Np,Ca,YZq),(Np,Ca,YZr),(Np,Ca,YZs),(Np,Ca,Y Zt),(Np,Ca,YZu),(Np,Ca,YZy),(Np,Ca,YZw),(Np,Ca,YZx),(Np,Ca,YZy),(Np,Ca,YZz),(Np,C a,YZaa),(Np,Ca,YZab),(Np,Ca,YZac),(Np,Ca,YZad),(Np,Ca,YZae),(Np,Ca,YZaf),(Np,Ca,Y Zag),(Np,Cb,YZa),(Np,Cb,YZb),(Np,Cb,YZc),(Np,Cb,YZd),(Np,Cb,YZe),(Np,Cb,YZf),(Np, Cb,YZg),(Np,Cb,YZh),(Np,Cb,YZi),(Np,Cb,YZj),(Np,Cb,YZk),(Np,Cb,YZl),(Np,Cb,YZm),( Np,Cb,YZn),(Np,Cb,YZo),(Np,Cb,YZp),(Np,Cb,YZq),(Np,Cb,YZr),(Np,Cb,YZs),(Np,Cb,Y Zt),(Np,Cb,YZu),(Np,Cb,YZv),(Np,Cb,YZw),(Np,Cb,YZx),(Np,Cb,YZy),(Np,Cb,YZz),(Np, Cb,YZaa),(Np,Cb,YZab),(Np,Cb,YZac),(Np,Cb,YZad),(Np,Cb,YZae),(Np,Cb,YZaf),(Np,Cb, YZag),(Np,Cc,YZa),(Np,Cc,YZb),(Np,Cc,YZc),(Np,Cc,YZd),(Np,Cc,YZe),(Np,Cc,YZf),(Np ,Cc,YZg),(Np,Cc,YZh),(Np,Cc,YZi),(Np,Cc,YZj),(Np,Cc,YZk),(Np,Cc,YZl),(Np,Cc,YZm),( Np,Cc,YZn),(Np,Cc,YZo),(Np,Cc,YZp),(Np,Cc,YZq),(Np,Cc,YZr),(Np,Cc,YZs),(Np,Cc,YZ t),(Np,Cc,YZu),(Np,Cc,YZv),(Np,Cc,YZw),(Np,Cc,YZx),(Np,Cc,YZy),(Np,Cc,YZz),(Np,Cc ,YZaa),(Np,Cc,YZab),(Np,Cc,YZac),(Np,Cc,YZad),(Np,Cc,YZae),(Np,Cc,YZaf),(Np,Cc,YZ ag),(Np,Cd,YZa),(Np,Cd,YZb),(Np,Cd,YZc),(Np,Cd,YZd),(Np,Cd,YZe),(Np,Cd,YZf),(Np,C d,YZg),(Np,Cd,YZh),(Np,Cd,YZi),(Np,Cd,YZj),(Np,Cd,YZk),(Np,Cd,YZl),(Np,Cd,YZm),( Np,Cd,YZn),(Np,Cd,YZo),(Np,Cd,YZp),(Np,Cd,YZq),(Np,Cd,YZr),(Np,Cd,YZs),(Np,Cd,Y Zt),(Np,Cd,YZu),(Np,Cd,YZy),(Np,Cd,YZw),(Np,Cd,YZx),(Np,Cd,YZy),(Np,Cd,YZz),(Np, Cd,YZaa),(Np,Cd,YZab),(Np,Cd,YZac),(Np,Cd,YZad),(Np,Cd,YZae),(Np,Cd,YZaf),(Np,Cd, YZag),(Np,Ce,YZa),(Np,Ce,YZb),(Np,Ce,YZc),(Np,Ce,YZd),(Np,Ce,YZe),(Np,Ce,YZf),(Np ,Ce,YZg),(Np,Ce,YZh),(Np,Ce,YZi),(Np,Ce,YZj),(Np,Ce,YZk),(Np,Ce,YZl),(Np,Ce,YZm),( Np,Ce,YZn),(Np,Ce,YZo),(Np,Ce,YZp),(Np,Ce,YZq),(Np,Ce,YZr),(Np,Ce,YZs),(Np,Ce,YZ t),(Np,Ce,YZu),(Np,Ce,YZv),(Np,Ce,YZw),(Np,Ce,YZx),(Np,Ce,YZy),(Np,Ce,YZz),(Np,Ce ,YZaa),(Np,Ce,YZab),(Np,Ce,YZac),(Np,Ce,YZad),(Np,Ce,YZae),(Np,Ce,YZaf),(Np,Ce,YZ ag),(Np,Cf,YZa),(Np,Cf,YZb),(Np,Cf,YZc),(Np,Cf,YZd),(Np,Cf,YZe),(Np,Cf,YZf),(Np,Cf, YZg),(Np,Cf,YZh),(Np,Cf,YZi),(Np,Cf,YZj),(Np,Cf,YZk),(Np,Cf,YZl),(Np,Cf,YZm),(Np,C f,YZn),(Np,Cf,YZo),(Np,Cf,YZp),(Np,Cf,YZq),(Np,Cf,YZr),(Np,Cf,YZs),(Np,Cf,YZt),(Np, Cf,YZu),(Np,Cf,YZy),(Np,Cf,YZw),(Np,Cf,YZx),(Np,Cf,YZy),(Np,Cf,YZz),(Np,Cf,YZaa),( Np,Cf,YZab),(Np,Cf,YZac),(Np,Cf,YZad),(Np,Cf,YZae),(Np,Cf,YZaf),(Np,Cf,YZag),(Np,C g,YZa),(Np,Cg,YZb),(Np,Cg,YZc),(Np,Cg,YZd),(Np,Cg,YZe),(Np,Cg,YZf),(Np,Cg,YZg),( Np,Cg,YZh),(Np,Cg,YZi),(Np,Cg,YZj),(Np,Cg,YZk),(Np,Cg,YZl),(Np,Cg,YZm),(Np,Cg,Y Zn),(Np,Cg,YZo),(Np,Cg,YZp),(Np,Cg,YZq),(Np,Cg,YZr),(Np,Cg,YZs),(Np,Cg,YZt),(Np,C g,YZu),(Np,Cg,YZy),(Np,Cg,YZw),(Np,Cg,YZx),(Np,Cg,YZy),(Np,Cg,YZz),(Np,Cg,YZaa), (Np,Cg,YZab),(Np,Cg,YZac),(Np,Cg,YZad),(Np,Cg,YZae),(Np,Cg,YZaf),(Np,Cg,YZag),(N p,Ch,YZa),(Np,Ch,YZb),(Np,Ch,YZc),(Np,Ch,YZd),(Np,Ch,YZe),(Np,Ch,YZf),(Np,Ch,YZg) ,(Np,Ch,YZh),(Np,Ch,YZi),(Np,Ch,YZj),(Np,Ch,Ya),(Np,Ch,YZl),(Np,Ch,YZm),(Np,Ch, YZn),(Np,Ch,YZo),(Np,Ch,YZp),(Np,Ch,YZq),(Np,Ch,YZr),(Np,Ch,YZs),(Np,Ch,YZt),(Np, Ch,YZu),(Np,Ch,YZy),(Np,Ch,YZw),(Np,Ch,YZx),(Np,Ch,YZy),(Np,Ch,YZz),(Np,Ch,YZa a),(Np,Ch,YZab),(Np,Ch,YZac),(Np,Ch,YZad),(Np,Ch,YZae),(Np,Ch,YZaf),(Np,Ch,YZag),( Np,Ci,YZa),(Np,Ci,YZb),(Np,Ci,YZc),(Np,Ci,YZd),(Np,Ci,YZe),(Np,Ci,YZf),(Np,Ci,YZg),( Np,Ci,YZh),(Np,Ci,YZi),(Np,Ci,YZj),(Np,Ci,YZk),(Np,Ci,YZl),(Np,Ci,YZm),(Np,Ci,YZn),( Np,Ci,YZo),(Np,Ci,YZp),(Np,Ci,YZq),(Np,Ci,YZr),(Np,Ci,YZs),(Np,Ci,YZt),(Np,Ci,YZu),( Np,Ci,YZy),(Np,Ci,YZw),(Np,Ci,YZx),(Np,Ci,YZy),(Np,Ci,YZz),(Np,Ci,YZaa),(Np,Ci,YZa b),(Np,Ci,YZac),(Np,Ci,YZad),(Np,Ci,YZae),(Np,Ci,YZaf),(Np,Ci,YZag),(Np,Cj,YZa),(Np, Cj,YZb),(Np,Cj,YZc),(Np,Cj,YZd),(Np,Cj,YZe),(Np,Cj,YZf),(Np,Cj,YZg),(Np,Cj,YZh),(Np ,Cj,YZi),(Np,Cj,YZj),(Np,Cj,YZk),(Np,Cj,YZl),(Np,Cj,YZm),(Np,Cj,YZn),(Np,Cj,YZo),(Np ,Cj,YZp),(Np,Cj,YZq),(Np,Cj,YZr),(Np,Cj,YZs),(Np,Cj,YZt),(Np,Cj,YZu),(Np,Cj,YZy),(Np ,Cj,YZw),(Np,Cj,YZx),(Np,Cj,YZy),(Np,Cj,YZz),(Np,Cj,YZaa),(Np,Cj,YZab),(Np,Cj,YZac) ,(Np,Cj,YZad),(Np,Cj,YZae),(Np,Cj,YZaf),(Np,Cj,YZag),(Nq,Ca,YZa),(Nq,Ca,YZb),(Nq,Ca ,YZc),(Nq,Ca,YZd),(Nq,Ca,YZe),(Nq,Ca,YZf),(Nq,Ca,YZg),(Nq,Ca,YZh),(Nq,Ca,YZi),(Nq, Ca,YZj),(Nq,Ca,YZk),(Nq,Ca,YZl),(Nq,Ca,YZm),(Nq,Ca,YZn),(Nq,Ca,YZo),(Nq,Ca,YZp),( Nq,Ca,YZq),(Nq,Ca,YZr),(Nq,Ca,YZs),(Nq,Ca,YZt),(Nq,Ca,YZu),(Nq,Ca,YZy),(Nq,Ca,YZ w),(Nq,Ca,YZx),(Nq,Ca,YZy),(Nq,Ca,YZz),(Nq,Ca,YZaa),(Nq,Ca,YZab),(Nq,Ca,YZac),(Nq ,Ca,YZad),(Nq,Ca,YZae),(Nq,Ca,YZaf),(Nq,Ca,YZag),(Nq,Cb,YZa),(Nq,Cb,YZb),(Nq,Cb,Y Zc),(Nq,Cb,YZd),(Nq,Cb,YZe),(Nq,Cb,YZf),(Nq,Cb,YZg),(Nq,Cb,YZh),(Nq,Cb,YZi),(Nq,C b,YZj),(Nq,Cb,YZk),(Nq,Cb,YZl),(Nq,Cb,YZm),(Nq,Cb,YZn),(Nq,Cb,YZo),(Nq,Cb,YZp),( Nq,Cb,YZq),(Nq,Cb,YZr),(Nq,Cb,YZs),(Nq,Cb,YZt),(Nq,Cb,YZu),(Nq,Cb,YZy),(Nq,Cb,YZ w),(Nq,Cb,YZx),(Nq,Cb,YZy),(Nq,Cb,YZz),(Nq,Cb,YZaa),(Nq,Cb,YZab),(Nq,Cb,YZac),(N q,Cb,YZad),(Nq,Cb,YZae),(Nq,Cb,YZaf),(Nq,Cb,YZag),(Nq,Cc,YZa),(Nq,Cc,YZb),(Nq,Cc, YZc),(Nq,Cc,YZd),(Nq,Cc,YZe),(Nq,Cc,YZf),(Nq,Cc,YZg),(Nq,Cc,YZh),(Nq,Cc,YZi),(Nq, Cc,YZj),(Nq,Cc,YZk),(Nq,Cc,YZl),(Nq,Cc,YZm),(Nq,Cc,YZn),(Nq,Cc,YZo),(Nq,Cc,YZp),( Nq,Cc,YZq),(Nq,Cc,YZr),(Nq,Cc,YZs),(Nq,Cc,YZt),(Nq,Cc,YZu),(Nq,Cc,YZy),(Nq,Cc,YZ w),(Nq,Cc,YZx),(Nq,Cc,YZy),(Nq,Cc,YZz),(Nq,Cc,YZaa),(Nq,Cc,YZab),(Nq,Cc,YZac),(Nq ,Cc,YZad),(Nq,Cc,YZae),(Nq,Cc,YZaf),(Nq,Cc,YZag),(Nq,Cd,YZa),(Nq,Cd,YZb),(Nq,Cd,Y Zc),(Nq,Cd,YZd),(Nq,Cd,YZe),(Nq,Cd,YZf),(Nq,Cd,YZg),(Nq,Cd,YZh),(Nq,Cd,YZi),(Nq,C d,YZj),(Nq,Cd,YZk),(Nq,Cd,YZl),(Nq,Cd,YZm),(Nq,Cd,YZn),(Nq,Cd,YZo),(Nq,Cd,YZp),( Nq,Cd,YZq),(Nq,Cd,YZr),(Nq,Cd,YZs),(Nq,Cd,YZt),(Nq,Cd,YZu),(Nq,Cd,YZy),(Nq,Cd,YZ w),(Nq,Cd,YZx),(Nq,Cd,YZy),(Nq,Cd,YZz),(Nq,Cd,YZaa),(Nq,Cd,YZab),(Nq,Cd,YZac),(N q,Cd,YZad),(Nq,Cd,YZae),(Nq,Cd,YZaf),(Nq,Cd,YZag),(Nq,Ce,YZa),(Nq,Ce,YZb),(Nq,Ce, YZc),(Nq,Ce,YZd),(Nq,Ce,YZe),(Nq,Ce,YZf),(Nq,Ce,YZg),(Nq,Ce,YZh),(Nq,Ce,YZi),(Nq, Ce,YZj),(Nq,Ce,YZk),(Nq,Ce,YZl),(Nq,Ce,YZm),(Nq,Ce,YZn),(Nq,Ce,YZo),(Nq,Ce,YZp),( Nq,Ce,YZq),(Nq,Ce,YZr),(Nq,Ce,YZs),(Nq,Ce,YZt),(Nq,Ce,YZu),(Nq,Ce,YZy),(Nq,Ce,YZ w),(Nq,Ce,YZx),(Nq,Ce,YZy),(Nq,Ce,YZz),(Nq,Ce,YZaa),(Nq,Ce,YZab),(Nq,Ce,YZac),(Nq ,Ce,YZad),(Nq,Ce,YZae),(Nq,Ce,YZaf),(Nq,Ce,YZag),(Nq,Cf,YZa),(Nq,Cf,YZb),(Nq,Cf,YZ c),(Nq,Cf,YZd),(Nq,Cf,YZe),(Nq,Cf,YZf),(Nq,Cf,YZg),(Nq,Cf,YZh),(Nq,Cf,YZi),(Nq,Cf,Y Zj),(Nq,Cf,YZk),(Nq,Cf,YZl),(Nq,Cf,YZm),(Nq,Cf,YZn),(Nq,Cf,YZo),(Nq,Cf,YZp),(Nq,Cf, YZq),(Nq,Cf,YZr),(Nq,Cf,YZs),(Nq,Cf,YZt),(Nq,Cf,YZu),(Nq,Cf,YZv),(Nq,Cf,YZw),(Nq,C f,YZx),(Nq,Cf,YZy),(Nq,Cf,YZz),(Nq,Cf,YZaa),(Nq,Cf,YZab),(Nq,Cf,YZac),(Nq,Cf,YZad), (Nq,Cf,YZae),(Nq,Cf,YZaf),(Nq,Cf,YZag),(Nq,Cg,YZa),(Nq,Cg,YZb),(Nq,Cg,YZc),(Nq,Cg, YZd),(Nq,Cg,YZe),(Nq,Cg,YZf),(Nq,Cg,YZg),(Nq,Cg,YZh),(Nq,Cg,YZi),(Nq,Cg,YZj),(Nq, Cg,YZk),(Nq,Cg,YZl),(Nq,Cg,YZm),(Nq,Cg,YZn),(Nq,Cg,YZo),(Nq,Cg,YZp),(Nq,Cg,YZq) ,(Nq,Cg,YZr),(Nq,Cg,YZs),(Nq,Cg,YZt),(Nq,Cg,YZu),(Nq,Cg,YZu),(Nq,Cg,YZw),(Nq,Cg, YZx),(Nq,Cg,YZy),(Nq,Cg,YZz),(Nq,Cg,YZaa),(Nq,Cg,YZab),(Nq,Cg,YZac),(Nq,Cg,YZad) ,(Nq,Cg,YZae),(Nq,Cg,YZaf),(Nq,Cg,YZag),(Nq,Ch,YZa),(Nq,Ch,YZb),(Nq,Ch,YZc),(Nq,C h,YZd),(Nq,Ch,YZe),(Nq,Ch,YZf),(Nq,Ch,YZg),(Nq,Ch,YZh),(Nq,Ch,YZi),(Nq,Ch,YZj),(N q,Ch,YZk),(Nq,Ch,YZl),(Nq,Ch,YZm),(Nq,Ch,YZn),(Nq,Ch,YZo),(Nq,Ch,YZp),(Nq,Ch,YZ q),(Nq,Ch,YZr),(Nq,Ch,YZs),(Nq,Ch,YZt),(Nq,Ch,YZu),(Nq,Ch,YZy),(Nq,Ch,YZw),(Nq,Ch ,YZx),(Nq,Ch,YZy),(Nq,Ch,YZz),(Nq,Ch,YZaa),(Nq,Ch,YZab),(Nq,Ch,YZac),(Nq,Ch,YZad) ,(Nq,Ch,YZae),(Nq,Ch,YZaf),(Nq,Ch,YZag),(Nq,Ci,YZa),(Nq,Ci,YZb),(Nq,Ci,YZc),(Nq,Ci ,YZd),(Nq,Ci,YZe),(Nq,Ci,YZf),(Nq,Ci,YZg),(Nq,Ci,YZh),(Nq,Ci,YZi),(Nq,Ci,YZj),(Nq,Ci, YZk),(Nq,Ci,YZl),(Nq,Ci,YZm),(Nq,Ci,YZn),(Nq,Ci,YZo),(Nq,Ci,YZp),(Nq,Ci,YZq),(Nq,C i,YZr),(Nq,Ci,YZs),(Nq,Ci,YZt),(Nq,Ci,YZu),(Nq,Ci,YZy),(Nq,Ci,YZw),(Nq,Ci,YZx),(Nq, Ci,YZy),(Nq,Ci,YZz),(Nq,Ci,YZaa),(Nq,Ci,YZab),(Nq,Ci,YZac),(Nq,Ci,YZad),(Nq,Ci,YZae) ,(Nq,Ci,YZaf),(Nq,Ci,YZag),(Nq,Cj,YZa),(Nq,Cj,YZb),(Nq,Cj,YZc),(Nq,Cj,YZd),(Nq,Cj,Y Ze),(Nq,Cj,YZf),(Nq,Cj,YZg),(Nq,Cj,YZh),(Nq,Cj,YZi),(Nq,Cj,YZj),(Nq,Cj,YZk),(Nq,Cj,Y Zl),(Nq,Cj,YZm),(Nq,Cj,YZn),(Nq,Cj,YZo),(Nq,Cj,YZp),(Nq,Cj,YZq),(Nq,Cj,YZr),(Nq,Cj, YZs),(Nq,Cj,YZt),(Nq,Cj,YZu),(Nq,Cj,YZy),(Nq,Cj,YZw),(Nq,Cj,YZx),(Nq,Cj,YZy),(Nq,Cj ,YZz),(Nq,Cj,YZaa),(Nq,Cj,YZab),(Nq,Cj,YZac),(Nq,Cj,YZad),(Nq,Cj,YZae),(Nq,Cj,YZaf), (Nq,Cj,YZag),(Nr,Ca,YZa),(Nr,Ca,YZb),(Nr,Ca,YZc),(Nr,Ca,YZd),(Nr,Ca,YZe),(Nr,Ca,YZf) ,(Nr,Ca,YZg),(Nr,Ca,YZh),(Nr,Ca,YZi),(Nr,Ca,YZj),(Nr,Ca,YZk),(Nr,Ca,YZl),(Nr,Ca,YZm) ,(Nr,Ca,YZn),(Nr,Ca,YZo),(Nr,Ca,YZp),(Nr,Ca,YZq),(Nr,Ca,YZr),(Nr,Ca,YZs),(Nr,Ca,YZt) ,(Nr,Ca,YZu),(Nr,Ca,YZy),(Nr,Ca,YZw),(Nr,Ca,YZx),(Nr,Ca,YZy),(Nr,Ca,YZz),(Nr,Ca,YZ aa),(Nr,Ca,YZab),(Nr,Ca,YZac),(Nr,Ca,YZad),(Nr,Ca,YZae),(Nr,Ca,YZaf),(Nr,Ca,YZag),(N r,Cb,YZa),(Nr,Cb,YZb),(Nr,Cb,YZc),(Nr,Cb,YZd),(Nr,Cb,YZe),(Nr,Cb,YZf),(Nr,Cb,YZg),( Nr,Cb,YZh),(Nr,Cb,YZi),(Nr,Cb,YZj),(Nr,Cb,YZk),(Nr,Cb,YZl),(Nr,Cb,YZm),(Nr,Cb,YZn), (Nr,Cb,YZo),(Nr,Cb,YZp),(Nr,Cb,YZq),(Nr,Cb,YZr),(Nr,Cb,YZs),(Nr,Cb,YZt),(Nr,Cb,YZu) ,(Nr,Cb,YZy),(Nr,Cb,YZw),(Nr,Cb,YZx),(Nr,Cb,YZy),(Nr,Cb,YZz),(Nr,Cb,YZaa),(Nr,Cb,Y Zab),(Nr,Cb,YZac),(Nr,Cb,YZad),(Nr,Cb,YZae),(Nr,Cb,YZaf),(Nr,Cb,YZag),(Nr,Cc,YZa),( Nr,Cc,YZb),(Nr,Cc,YZc),(Nr,Cc,YZd),(Nr,Cc,YZe),(Nr,Cc,YZf),(Nr,Cc,YZg),(Nr,Cc,YZh), (Nr,Cc,YZi),(Nr,Cc,YZj),(Nr,Cc,YZk),(Nr,Cc,YZl),(Nr,Cc,YZm),(Nr,Cc,YZn),(Nr,Cc,YZo), (Nr,Cc,YZp),(Nr,Cc,YZq),(Nr,Cc,YZr),(Nr,Cc,YZs),(Nr,Cc,YZt),(Nr,Cc,YZu),(Nr,Cc,YZy), (Nr,Cc,YZw),(Nr,Cc,YZx),(Nr,Cc,YZy),(Nr,Cc,YZz),(Nr,Cc,YZaa),(Nr,Cc,YZab),(Nr,Cc,Y Zac),(Nr,Cc,YZad),(Nr,Cc,YZae),(Nr,Cc,YZaf),(Nr,Cc,YZag),(Nr,Cd,YZa),(Nr,Cd,YZb),(Nr ,Cd,YZc),(Nr,Cd,YZd),(Nr,Cd,YZe),(Nr,Cd,YZf),(Nr,Cd,YZg),(Nr,Cd,YZh),(Nr,Cd,YZi),(N r,Cd,YZj),(Nr,Cd,YZk),(Nr,Cd,YZl),(Nr,Cd,YZm),(Nr,Cd,YZn),(Nr,Cd,YZo),(Nr,Cd,YZp),( Nr,Cd,YZq),(Nr,Cd,YZr),(Nr,Cd,YZs),(Nr,Cd,YZt),(Nr,Cd,YZu),(Nr,Cd,YZy),(Nr,Cd,YZw) ,(Nr,Cd,YZx),(Nr,Cd,YZy),(Nr,Cd,YZz),(Nr,Cd,YZaa),(Nr,Cd,YZab),(Nr,Cd,YZac),(Nr,Cd, YZad),(Nr,Cd,YZae),(Nr,Cd,YZaf),(Nr,Cd,YZag),(Nr,Ce,YZa),(Nr,Ce,YZb),(Nr,Ce,YZc),(N r,Ce,YZd),(Nr,Ce,YZe),(Nr,Ce,YZf),(Nr,Ce,YZg),(Nr,Ce,YZh),(Nr,Ce,YZi),(Nr,Ce,YZj),(Nr ,Ce,YZk),(Nr,Ce,YZl),(Nr,Ce,YZm),(Nr,Ce,YZn),(Nr,Ce,YZo),(Nr,Ce,YZp),(Nr,Ce,YZq),( Nr,Ce,YZr),(Nr,Ce,YZs),(Nr,Ce,YZt),(Nr,Ce,YZu),(Nr,Ce,YZv),(Nr,Ce,YZw),(Nr,Ce,YZx), (Nr,Ce,YZy),(Nr,Ce,YZz),(Nr,Ce,YZaa),(Nr,Ce,YZab),(Nr,Ce,YZac),(Nr,Ce,YZad),(Nr,Ce, YZae),(Nr,Ce,YZaf),(Nr,Ce,YZag),(Nr,Cf,YZa),(Nr,Cf,YZb),(Nr,Cf,YZc),(Nr,Cf,YZd),(Nr, Cf,YZe),(Nr,Cf,YZf),(Nr,Cf,YZg),(Nr,Cf,YZh),(Nr,Cf,YZi),(Nr,Cf,YZj),(Nr,Cf,YZk),(Nr,Cf ,YZl),(Nr,Cf,YZm),(Nr,Cf,YZn),(Nr,Cf,YZo),(Nr,Cf,YZp),(Nr,Cf,YZq),(Nr,Cf,YZr),(Nr,Cf, YZs),(Nr,Cf,YZt),(Nr,Cf,YZu),(Nr,Cf,YZy),(Nr,Cf,YZw),(Nr,Cf,YZx),(Nr,Cf,YZy),(Nr,Cf, YZz),(Nr,Cf,YZaa),(Nr,Cf,YZab),(Nr,Cf,YZac),(Nr,Cf,YZad),(Nr,Cf,YZae),(Nr,Cf,YZaf),(N r,Cf,YZag),(Nr,Cg,YZa),(Nr,Cg,YZb),(Nr,Cg,YZc),(Nr,Cg,YZd),(Nr,Cg,YZe),(Nr,Cg,YZf),( Nr,Cg,YZg),(Nr,Cg,YZh),(Nr,Cg,YZi),(Nr,Cg,YZj),(Nr,Cg,YZk),(Nr,Cg,YZl),(Nr,Cg,YZm), (Nr,Cg,YZn),(Nr,Cg,YZo),(Nr,Cg,YZp),(Nr,Cg,YZq),(Nr,Cg,YZr),(Nr,Cg,YZs),(Nr,Cg,YZt) ,(Nr,Cg,YZu),(Nr,Cg,YZy),(Nr,Cg,YZw),(Nr,Cg,YZx),(Nr,Cg,YZy),(Nr,Cg,YZz),(Nr,Cg,Y Zaa),(Nr,Cg,YZab),(Nr,Cg,YZac),(Nr,Cg,YZad),(Nr,Cg,YZae),(Nr,Cg,YZaf),(Nr,Cg,YZag), (Nr,Ch,YZa),(Nr,Ch,YZb),(Nr,Ch,YZc),(Nr,Ch,YZd),(Nr,Ch,YZe),(Nr,Ch,YZf),(Nr,Ch,YZg) ,(Nr,Ch,YZh),(Nr,Ch,YZi),(Nr,Ch,YZj),(Nr,Ch,YZk),(Nr,Ch,YZl),(Nr,Ch,YZm),(Nr,Ch,YZ n),(Nr,Ch,YZo),(Nr,Ch,YZp),(Nr,Ch,YZq),(Nr,Ch,YZr),(Nr,Ch,YZs),(Nr,Ch,YZt),(Nr,Ch,Y Zu),(Nr,Ch,YZv),(Nr,Ch,YZw),(Nr,Ch,YZx),(Nr,Ch,YZy),(Nr,Ch,YZz),(Nr,Ch,YZaa),(Nr,C h,YZab),(Nr,Ch,YZac),(Nr,Ch,YZad),(Nr,Ch,YZae),(Nr,Ch,YZaf),(Nr,Ch,YZag),(Nr,Ci,YZa) ,(Nr,Ci,YZb),(Nr,Ci,YZc),(Nr,Ci,YZd),(Nr,Ci,YZe),(Nr,Ci,YZf),(Nr,Ci,YZg),(Nr,Ci,YZh),( Nr,Ci,YZi),(Nr,Ci,YZj),(Nr,Ci,YZk),(Nr,Ci,YZl),(Nr,Ci,YZm),(Nr,Ci,YZn),(Nr,Ci,YZo),(Nr ,Ci,YZp),(Nr,Ci,YZq),(Nr,Ci,YZr),(Nr,Ci,YZs),(Nr,Ci,YZt),(Nr,Ci,YZu),(Nr,Ci,YZy),(Nr,Ci ,YZw),(Nr,Ci,YZx),(Nr,Ci,YZy),(Nr,Ci,YZz),(Nr,Ci,YZaa),(Nr,Ci,YZab),(Nr,Ci,YZac),(Nr, Ci,YZad),(Nr,Ci,YZae),(Nr,Ci,YZaf),(Nr,Ci,YZag),(Nr,Cj,YZa),(Nr,Cj,YZb),(Nr,Cj,YZc),(N r,Cj,YZd),(Nr,Cj,YZe),(Nr,Cj,YZf),(Nr,Cj,YZg),(Nr,Cj,YZl),(Nr,Cj,YZi),(Nr,Cj,YZj),(Nr,Cj ,YZk),(Nr,Cj,YZl),(Nr,Cj,YZm),(Nr,Cj,YZn),(Nr,Cj,YZo),(Nr,Cj,YZp),(Nr,Cj,YZq),(Nr,Cj, YZr),(Nr,Cj,YZs),(Nr,Cj,YZt),(Nr,Cj,YZu),(Nr,Cj,YZy),(Nr,Cj,YZw),(Nr,Cj,YZx),(Nr,Cj,Y Zy),(Nr,Cj,YZz),(Nr,Cj,YZaa),(Nr,Cj,YZab),(Nr,Cj,YZac),(Nr,Cj,YZad),(Nr,Cj,YZae),(Nr,C j,YZaf),(Nr,Cj,YZag),(Ns,Ca,YZa),(Ns,Ca,YZb),(Ns,Ca,YZc),(Ns,Ca,YZd),(Ns,Ca,YZe),(Ns ,Ca,YZf),(Ns,Ca,YZg),(Ns,Ca,YZh),(Ns,Ca,YZi),(Ns,Ca,YZj),(Ns,Ca,YZk),(Ns,Ca,YZl),(Ns ,Ca,YZm),(Ns,Ca,YZn),(Ns,Ca,YZo),(Ns,Ca,YZp),(Ns,Ca,YZq),(Ns,Ca,YZr),(Ns,Ca,YZs),( Ns,Ca,YZt),(Ns,Ca,YZu),(Ns,Ca,YZy),(Ns,Ca,YZw),(Ns,Ca,YZx),(Ns,Ca,YZy),(Ns,Ca,YZz) ,(Ns,Ca,YZaa),(Ns,Ca,YZab),(Ns,Ca,YZac),(Ns,Ca,YZad),(Ns,Ca,YZae),(Ns,Ca,YZaf),(Ns, Ca,YZag),(Ns,Cb,YZa),(Ns,Cb,YZb),(Ns,Cb,YZc),(Ns,Cb,YZd),(Ns,Cb,YZe),(Ns,Cb,YZf),( Ns,Cb,YZg),(Ns,Cb,YZh),(Ns,Cb,YZi),(Ns,Cb,YZj),(Ns,Cb,YZk),(Ns,Cb,YZl),(Ns,Cb,YZm) ,(Ns,Cb,YZn),(Ns,Cb,YZo),(Ns,Cb,YZp),(Ns,Cb,YZq),(Ns,Cb,YZr),(Ns,Cb,YZs),(Ns,Cb,Y Zt),(Ns,Cb,YZu),(Ns,Cb,YZy),(Ns,Cb,YZw),(Ns,Cb,YZx),(Ns,Cb,YZy),(Ns,Cb,YZz),(Ns,C b,YZaa),(Ns,Cb,YZab),(Ns,Cb,YZac),(Ns,Cb,YZad),(Ns,Cb,YZae),(Ns,Cb,YZaf),(Ns,Cb,YZ ag),(Ns,Cc,YZa),(Ns,Cc,YZb),(Ns,Cc,YZc),(Ns,Cc,YZd),(Ns,Cc,YZe),(Ns,Cc,YZf),(Ns,Cc, YZg),(Ns,Cc,YZh),(Ns,Cc,YZi),(Ns,Cc,YZj),(Ns,Cc,YZk),(Ns,Cc,YZl),(Ns,Cc,YZm),(Ns,C c,YZn),(Ns,Cc,YZo),(Ns,Cc,YZp),(Ns,Cc,YZq),(Ns,Cc,YZr),(Ns,Cc,YZs),(Ns,Cc,YZt),(Ns, Cc,YZu),(Ns,Cc,YZy),(Ns,Cc,YZw),(Ns,Cc,YZx),(Ns,Cc,YZy),(Ns,Cc,YZz),(Ns,Cc,YZaa),( Ns,Cc,YZab),(Ns,Cc,YZac),(Ns,Cc,YZad),(Ns,Cc,YZae),(Ns,Cc,YZaf),(Ns,Cc,YZag),(Ns,C d,YZa),(Ns,Cd,YZb),(Ns,Cd,YZc),(Ns,Cd,YZd),(Ns,Cd,YZe),(Ns,Cd,YZf),(Ns,Cd,YZg),(Ns ,Cd,YZh),(Ns,Cd,YZi),(Ns,Cd,YZj),(Ns,Cd,YZlc),(Ns,Cd,YZl),(Ns,Cd,YZm),(Ns,Cd,YZn),( Ns,Cd,YZo),(Ns,Cd,YZp),(Ns,Cd,YZq),(Ns,Cd,YZr),(Ns,Cd,YZs),(Ns,Cd,YZt),(Ns,Cd,YZu) ,(Ns,Cd,YZy),(Ns,Cd,YZw),(Ns,Cd,YZx),(Ns,Cd,YZy),(Ns,Cd,YZz),(Ns,Cd,YZaa),(Ns,Cd, YZab),(Ns,Cd,YZac),(Ns,Cd,YZad),(Ns,Cd,YZae),(Ns,Cd,YZaf),(Ns,Cd,YZag),(Ns,Ce,YZa) ,(Ns,Ce,YZb),(Ns,Ce,YZc),(Ns,Ce,YZd),(Ns,Ce,YZe),(Ns,Ce,YZf),(Ns,Ce,YZg),(Ns,Ce,YZ h),(Ns,Ce,YZi),(Ns,Ce,YZj),(Ns,Ce,YZk),(Ns,Ce,YZl),(Ns,Ce,YZm),(Ns,Ce,YZn),(Ns,Ce,Y Zo),(Ns,Ce,YZp),(Ns,Ce,YZq),(Ns,Ce,YZr),(Ns,Ce,YZs),(Ns,Ce,YZt),(Ns,Ce,YZu),(Ns,Ce, YZv),(Ns,Ce,YZw),(Ns,Ce,YZx),(Ns,Ce,YZy),(Ns,Ce,YZz),(Ns,Ce,YZaa),(Ns,Ce,YZab),(N s,Ce,YZac),(Ns,Ce,YZad),(Ns,Ce,YZae),(Ns,Ce,YZaf),(Ns,Ce,YZag),(Ns,Cf,YZa),(Ns,Cf,Y Zb),(Ns,Cf,YZc),(Ns,Cf,YZd),(Ns,Cf,YZe),(Ns,Cf,YZf),(Ns,Cf,YZg),(Ns,Cf,YZh),(Ns,Cf,Y Zi),(Ns,Cf,YZj),(Ns,Cf,YZk),(Ns,Cf,YZl),(Ns,Cf,YZm),(Ns,Cf,YZn),(Ns,Cf,YZo),(Ns,Cf,Y Zp),(Ns,Cf,YZq),(Ns,Cf,YZr),(Ns,Cf,YZs),(Ns,Cf,YZf),(Ns,Cf,YZu),(Ns,Cf,YZy),(Ns,Cf,Y Zw),(Ns,Cf,YZx),(Ns,Cf,YZy),(Ns,Cf,YZz),(Ns,Cf,YZaa),(Ns,Cf,YZab),(Ns,Cf,YZac),(Ns,C f,YZad),(Ns,Cf,YZae),(Ns,Cf,YZaf),(Ns,Cf,YZag),(Ns,Cg,YZa),(Ns,Cg,YZb),(Ns,Cg,YZc),( Ns,Cg,YZd),(Ns,Cg,YZe),(Ns,Cg,YZf),(Ns,Cg,YZg),(Ns,Cg,YZh),(Ns,Cg,YZi),(Ns,Cg,YZj) ,(Ns,Cg,YZl),(Ns,Cg,YZl),(Ns,Cg,YZm),(Ns,Cg,YZn),(Ns,Cg,YZo),(Ns,Cg,YZp),(Ns,Cg,Y Zq),(Ns,Cg,YZr),(Ns,Cg,YZs),(Ns,Cg,YZt),(Ns,Cg,YZu),(Ns,Cg,YZy),(Ns,Cg,YZw),(Ns,Cg ,YZx),(Ns,Cg,YZy),(Ns,Cg,YZz),(Ns,Cg,YZaa),(Ns,Cg,YZab),(Ns,Cg,YZac),(Ns,Cg,YZad), (Ns,Cg,YZae),(Ns,Cg,YZaf),(Ns,Cg,YZag),(Ns,Ch,YZa),(Ns,Ch,YZb),(Ns,Ch,YZc),(Ns,Ch, YZd),(Ns,Ch,YZe),(Ns,Ch,YZf),(Ns,Ch,YZg),(Ns,Ch,YZh),(Ns,Ch,YZi),(Ns,Ch,YZj),(Ns,C h,YZk),(Ns,Ch,YZl),(Ns,Ch,YZm),(Ns,Ch,YZn),(Ns,Ch,YZo),(Ns,Ch,YZp),(Ns,Ch,YZq),(N s,Ch,YZr),(Ns,Ch,YZs),(Ns,Ch,YZt),(Ns,Ch,YZu),(Ns,Ch,YZy),(Ns,Ch,YZw),(Ns,Ch,YZx), (Ns,Ch,YZy),(Ns,Ch,YZz),(Ns,Ch,YZaa),(Ns,Ch,YZab),(Ns,Ch,YZac),(Ns,Ch,YZad),(Ns,C h,YZae),(Ns,Ch,YZaf),(Ns,Ch,YZag),(Ns,Ci,YZa),(Ns,Ci,YZb),(Ns,Ci,YZc),(Ns,Ci,YZd),(N s,Ci,YZe),(Ns,Ci,YZf),(Ns,Ci,YZg),(Ns,Ci,YZh),(Ns,Ci,YZi),(Ns,Ci,YZj),(Ns,Ci,YZk),(Ns, Ci,YZl),(Ns,Ci,YZm),(Ns,Ci,YZn),(Ns,Ci,YZo),(Ns,Ci,YZp),(Ns,Ci,YZq),(Ns,Ci,YZr),(Ns, Ci,YZs),(Ns,Ci,YZt),(Ns,Ci,YZu),(Ns,Ci,YZy),(Ns,Ci,YZw),(Ns,Ci,YZx),(Ns,Ci,YZy),(Ns, Ci,YZz),(Ns,Ci,YZaa),(Ns,Ci,YZab),(Ns,Ci,YZac),(Ns,Ci,YZad),(Ns,Ci,YZae),(Ns,Ci,YZaf) ,(Ns,Ci,YZag),(Ns,Ci,YZa),(Ns,Ci,YZb),(Ns,Ci,YZc),(Ns,Ci,YZd),(Ns,Ci,YZe),(Ns,Ci,YZD, (Ns,Ci,YZg),(Ns,Ci,YZh),(Ns,Ci,YZi),(Ns,Ci,YZj),(Ns,Ci,YZk),(Ns,Ci,YZl),(Ns,Ci,YZm),( Ns,Ci,YZn),(Ns,Ci,YZo),(Ns,Ci,YZp),(Ns,Ci,YZq),(Ns,Ci,YZr),(Ns,Ci,YZs),(Ns,Ci,YZt),(N s,Ci,YZu),(Ns,Ci,YZv),(Ns,Ci,YZw),(Ns,Ci,YZx),(Ns,Ci,YZy),(Ns,Ci,YZz),(Ns,Ci,YZaa),( Ns,Ci,YZab),(Ns,Ci,YZac),(Ns,Ci,YZad),(Ns,Ci,YZae),(Ns,Ci,YZaf),(Ns,Ci,YZag),(Nt,Ca,Y Za),(Nt,Ca,YZb),(Nt,Ca,YZc),(Nt,Ca,YZd),(Nt,Ca,YZe),(Nt,Ca,YZf),(Nt,Ca,YZg),(Nt,Ca,Y Zh),(Nt,Ca,YZi),(Nt,Ca,YZj),(Nt,Ca,YZk),(Nt,Ca,YZl),(Nt,Ca,YZm),(Nt,Ca,YZn),(Nt,Ca,Y Zo),(Nt,Ca,YZp),(Nt,Ca,YZq),(Nt,Ca,YZr),(Nt,Ca,YZs),(Nt,Ca,YZt),(Nt,Ca,YZu),(Nt,Ca,Y Zv),(Nt,Ca,YZw),(Nt,Ca,YZx),(Nt,Ca,YZy),(Nt,Ca,YZz),(Nt,Ca,YZaa),(Nt,Ca,YZab),(Nt,Ca ,YZac),(Nt,Ca,YZad),(Nt,Ca,YZae),(Nt,Ca,YZaf),(Nt,Ca,YZag),(Nt,Cb,YZa),(Nt,Cb,YZb),( Nt,Cb,YZc),(Nt,Cb,YZd),(Nt,Cb,YZe),(Nt,Cb,YZf),(Nt,Cb,YZg),(Nt,Cb,YZh),(Nt,Cb,YZi),( Nt,Cb,YZj),(Nt,Cb,YZk),(Nt,Cb,YZl),(Nt,Cb,YZm),(Nt,Cb,YZn),(Nt,Cb,YZo),(Nt,Cb,YZp), (Nt,Cb,YZq),(Nt,Cb,YZr),(Nt,Cb,YZs),(Nt,Cb,YZt),(Nt,Cb,YZu),(Nt,Cb,YZv),(Nt,Cb,YZw) ,(Nt,Cb,YZx),(Nt,Cb,YZy),(Nt,Cb,YZz),(Nt,Cb,YZaa),(Nt,Cb,YZab),(Nt,Cb,YZac),(Nt,Cb, YZad),(Nt,Cb,YZae),(Nt,Cb,YZaf),(Nt,Cb,YZag),(Nt,Cc,YZa),(Nt,Cc,YZb),(Nt,Cc,YZc),(Nt ,Cc,YZd),(Nt,Cc,YZe),(Nt,Cc,YZf),(Nt,Cc,YZg),(Nt,Cc,YZh),(Nt,Cc,YZi),(Nt,Cc,YZj),(Nt, Cc,YZk),(Nt,Cc,YZl),(Nt,Cc,YZm),(Nt,Cc,YZn),(Nt,Cc,YZo),(Nt,Cc,YZp),(Nt,Cc,YZq),(Nt, Cc,YZr),(Nt,Cc,YZs),(Nt,Cc,YZt),(Nt,Cc,YZu),(Nt,Cc,YZv),(Nt,Cc,YZw),(Nt,Cc,YZx),(Nt, Cc,YZy),(Nt,Cc,YZz),(Nt,Cc,YZaa),(Nt,Cc,YZab),(Nt,Cc,YZac),(Nt,Cc,YZad),(Nt,Cc,YZae) ,(Nt,Cc,YZaf),(Nt,Cc,YZag),(Nt,Cd,YZa),(Nt,Cd,YZb),(Nt,Cd,YZc),(Nt,Cd,Yal),(Nt,Cd,Y Ze),(Nt,Cd,YZf),(Nt,Cd,YZg),(Nt,Cd,YZh),(Nt,Cd,YZi),(Nt,Cd,YZj),(Nt,Cd,YZk),(Nt,Cd,Y Zl),(Nt,Cd,YZm),(Nt,Cd,YZn),(Nt,Cd,YZo),(Nt,Cd,YZp),(Nt,Cd,YZq),(Nt,Cd,YZr),(Nt,Cd, YZs),(Nt,Cd,YZt),(Nt,Cd,YZu),(Nt,Cd,YZv),(Nt,Cd,YZw),(Nt,Cd,YZx),(Nt,Cd,YZy),(Nt,Cd ,YZz),(Nt,Cd,YZaa),(Nt,Cd,YZab),(Nt,Cd,YZac),(Nt,Cd,YZad),(Nt,Cd,YZae),(Nt,Cd,YZaf), (Nt,Cd,YZag),(Nt,Ce,YZa),(Nt,Ce,YZb),(Nt,Ce,YZc),(Nt,Ce,YZd),(Nt,Ce,YZe),(Nt,Ce,YZf) ,(Nt,Ce,YZg),(Nt,Ce,YZh),(Nt,Ce,YZi),(Nt,Ce,YZj),(Nt,Ce,YZk),(Nt,Ce,YZl),(Nt,Ce,YZm), (Nt,Ce,YZn),(Nt,Ce,YZo),(Nt,Ce,YZp),(Nt,Ce,YZq),(Nt,Ce,YZr),(Nt,Ce,YZs),(Nt,Ce,YZt),( Nt,Ce,YZu),(Nt,Ce,YZv),(Nt,Ce,YZw),(Nt,Ce,YZx),(Nt,Ce,YZy),(Nt,Ce,YZz),(Nt,Ce,YZaa) ,(Nt,Ce,YZab),(Nt,Ce,YZac),(Nt,Ce,YZad),(Nt,Ce,YZae),(Nt,Ce,YZaf),(Nt,Ce,YZag),(Nt,Cf ,YZa),(Nt,Cf,YZb),(Nt,Cf,YZc),(Nt,Cf,YZd),(Nt,Cf,YZe),(Nt,Cf,YZf),(Nt,Cf,YZg),(Nt,Cf,Y Zh),(Nt,Cf,YZi),(Nt,Cf,YZj),(Nt,Cf,YZk),(Nt,Cf,YZl),(Nt,Cf,YZm),(Nt,Cf,YZn),(Nt,Cf,YZo) ,(Nt,Cf,YZp),(Nt,Cf,YZq),(Nt,Cf,YZr),(Nt,Cf,YZs),(Nt,Cf,YZt),(Nt,Cf,YZu),(Nt,Cf,YZv),( Nt,Cf,YZw),(Nt,Cf,YZx),(Nt,Cf,YZy),(Nt,Cf,YZz),(Nt,Cf,YZaa),(Nt,Cf,YZab),(Nt,Cf,YZac) ,(Nt,Cf,YZad),(Nt,Cf,YZae),(Nt,Cf,YZaf),(Nt,Cf,YZag),(Nt,Cg,YZa),(Nt,Cg,YZb),(Nt,Cg,Y Zc),(Nt,Cg,YZd),(Nt,Cg,YZe),(Nt,Cg,YZf),(Nt,Cg,YZg),(Nt,Cg,YZh),(Nt,Cg,YZi),(Nt,Cg,Y Zj),(Nt,Cg,YZk),(Nt,Cg,YZl),(Nt,Cg,YZm),(Nt,Cg,YZn),(Nt,Cg,YZo),(Nt,Cg,YZp),(Nt,Cg, YZq),(Nt,Cg,YZr),(Nt,Cg,YZs),(Nt,Cg,YZt),(Nt,Cg,YZu),(Nt,Cg,YZv),(Nt,Cg,YZw),(Nt,Cg ,YZx),(Nt,Cg,YZy),(Nt,Cg,YZz),(Nt,Cg,YZaa),(Nt,Cg,YZab),(Nt,Cg,YZac),(Nt,Cg,YZad),( Nt,Cg,YZae),(Nt,Cg,YZaf),(Nt,Cg,YZag),(Nt,Ch,YZa),(Nt,Ch,YZb),(Nt,Ch,YZc),(Nt,Ch,YZ d),(Nt,Ch,YZe),(Nt,Ch,YZf),(Nt,Ch,YZg),(Nt,Ch,YZh),(Nt,Ch,YZi),(Nt,Ch,YZj),(Nt,Ch,YZ k),(Nt,Ch,YZl),(Nt,Ch,YZm),(Nt,Ch,YZn),(Nt,Ch,YZo),(Nt,Ch,YZp),(Nt,Ch,YZq),(Nt,Ch,Y Zr),(Nt,Ch,YZs),(Nt,Ch,YZt),(Nt,Ch,YZu),(Nt,Ch,YZv),(Nt,Ch,YZw),(Nt,Ch,YZx),(Nt,Ch, YZy),(Nt,Ch,YZz),(Nt,Ch,YZaa),(Nt,Ch,YZab),(Nt,Ch,YZac),(Nt,Ch,YZad),(Nt,Ch,YZae),( Nt,Ch,YZaf),(Nt,Ch,YZag),(Nt,Ci,YZa),(Nt,Ci,YZb),(Nt,Ci,YZc),(Nt,Ci,YZd),(Nt,Ci,YZe),( Nt,Ci,YZf),(Nt,Ci,YZg),(Nt,Ci,YZh),(Nt,Ci,YZi),(Nt,Ci,YZj),(Nt,Ci,YZk),(Nt,Ci,YZl),(Nt,C i,YZm),(Nt,Ci,YZn),(Nt,Ci,YZo),(Nt,Ci,YZp),(Nt,Ci,YZq),(Nt,Ci,YZr),(Nt,Ci,YZs),(Nt,Ci,Y Zt),(Nt,Ci,YZu),(Nt,Ci,YZv),(Nt,Ci,YZw),(Nt,Ci,YZx),(Nt,Ci,YZy),(Nt,Ci,YZz),(Nt,Ci,YZa a),(Nt,Ci,YZab),(Nt,Ci,YZac),(Nt,Ci,YZad),(Nt,Ci,YZae),(Nt,Ci,YZaf),(Nt,Ci,YZag),(Nt,Cj, YZa),(Nt,Cj,YZb),(Nt,Cj,YZc),(Nt,Cj,YZd),(Nt,Cj,YZe),(Nt,Cj,YZf),(Nt,Cj,YZg),(Nt,Cj,YZ h),(Nt,Cj,YZi),(Nt,Cj,YZj),(Nt,Cj,YZk),(Nt,Cj,YZl),(Nt,Cj,YZm),(Nt,Cj,YZn),(Nt,Cj,YZo),( Nt,Cj,YZp),(Nt,Cj,YZq),(Nt,Cj,YZr),(Nt,Cj,YZs),(Nt,Cj,YZt),(Nt,Cj,YZu),(Nt,Cj,YZy),(Nt, Cj,YZw),(Nt,Cj,YZx),(Nt,Cj,YZy),(Nt,Cj,YZz),(Nt,Cj,YZaa),(Nt,Cj,YZab),(Nt,Cj,YZac),(Nt ,Cj,YZad),(Nt,Cj,YZae),(Nt,Cj,YZaf),(Nt,Cj,YZag),(Nu,Ca,YZa),(Nu,Ca,YZb),(Nu,Ca,YZc) ,(Nu,Ca,YZd),(Nu,Ca,YZe),(Nu,Ca,YZf),(Nu,Ca,YZg),(Nu,Ca,YZh),(Nu,Ca,YZi),(Nu,Ca,Y Zj),(Nu,Ca,YZk),(Nu,Ca,YZl),(Nu,Ca,YZm),(Nu,Ca,YZn),(Nu,Ca,YZo),(Nu,Ca,YZp),(Nu,C a,YZq),(Nu,Ca,YZr),(Nu,Ca,YZs),(Nu,Ca,YZt),(Nu,Ca,YZu),(Nu,Ca,YZy),(Nu,Ca,YZw),(N u,Ca,YZx),(Nu,Ca,YZy),(Nu,Ca,YZz),(Nu,Ca,YZaa),(Nu,Ca,YZab),(Nu,Ca,YZac),(Nu,Ca,Y Zad),(Nu,Ca,YZae),(Nu,Ca,YZaf),(Nu,Ca,YZag),(Nu,Cb,YZa),(Nu,Cb,YZb),(Nu,Cb,YZc),( Nu,Cb,YZd),(Nu,Cb,YZe),(Nu,Cb,YZf),(Nu,Cb,YZg),(Nu,Cb,YZh),(Nu,Cb,YZi),(Nu,Cb,YZ j),(Nu,Cb,YZk),(Nu,Cb,YZl),(Nu,Cb,YZm),(Nu,Cb,YZn),(Nu,Cb,YZo),(Nu,Cb,YZp),(Nu,C b,YZq),(Nu,Cb,YZr),(Nu,Cb,YZs),(Nu,Cb,YZt),(Nu,Cb,YZu),(Nu,Cb,YZy),(Nu,Cb,YZw),( Nu,Cb,YZx),(Nu,Cb,YZy),(Nu,Cb,YZz),(Nu,Cb,YZaa),(Nu,Cb,YZab),(Nu,Cb,YZac),(Nu,C b,YZad),(Nu,Cb,YZae),(Nu,Cb,YZaf),(Nu,Cb,YZag),(Nu,Cc,YZa),(Nu,Cc,YZb),(Nu,Cc,YZ c),(Nu,Cc,YZd),(Nu,Cc,YZe),(Nu,Cc,YZf),(Nu,Cc,YZg),(Nu,Cc,YZh),(Nu,Cc,YZi),(Nu,Cc, YZj),(Nu,Cc,YZk),(Nu,Cc,YZl),(Nu,Cc,YZm),(Nu,Cc,YZn),(Nu,Cc,YZo),(Nu,Cc,YZp),(Nu, Cc,YZq),(Nu,Cc,YZr),(Nu,Cc,YZs),(Nu,Cc,YZt),(Nu,Cc,YZu),(Nu,Cc,YZy),(Nu,Cc,YZw),( Nu,Cc,YZx),(Nu,Cc,YZy),(Nu,Cc,YZz),(Nu,Cc,YZaa),(Nu,Cc,YZab),(Nu,Cc,YZac),(Nu,Cc, YZad),(Nu,Cc,YZae),(Nu,Cc,YZaf),(Nu,Cc,YZag),(Nu,Cd,YZa),(Nu,Cd,YZb),(Nu,Cd,YZc), (Nu,Cd,YZd),(Nu,Cd,YZe),(Nu,Cd,YZf),(Nu,Cd,YZg),(Nu,Cd,YZh),(Nu,Cd,YZi),(Nu,Cd,Y Zj),(Nu,Cd,Ya),(Nu,Cd,YZl),(Nu,Cd,YZm),(Nu,Cd,YZn),(Nu,Cd,YZo),(Nu,Cd,YZp),(Nu, Cd,YZq),(Nu,Cd,YZr),(Nu,Cd,YZs),(Nu,Cd,YZt),(Nu,Cd,YZu),(Nu,Cd,YZy),(Nu,Cd,YZw), (Nu,Cd,YZx),(Nu,Cd,YZy),(Nu,Cd,YZz),(Nu,Cd,YZaa),(Nu,Cd,YZab),(Nu,Cd,YZac),(Nu,C d,YZad),(Nu,Cd,YZae),(Nu,Cd,YZaf),(Nu,Cd,YZag),(Nu,Ce,YZa),(Nu,Ce,YZb),(Nu,Ce,YZ c),(Nu,Ce,YZd),(Nu,Ce,YZe),(Nu,Ce,YZf),(Nu,Ce,YZg),(Nu,Ce,YZh),(Nu,Ce,YZi),(Nu,Ce, YZj),(Nu,Ce,YZk),(Nu,Ce,YZl),(Nu,Ce,YZm),(Nu,Ce,YZn),(Nu,Ce,YZo),(Nu,Ce,YZp),(Nu, Ce,YZq),(Nu,Ce,YZr),(Nu,Ce,YZs),(Nu,Ce,YZt),(Nu,Ce,YZu),(Nu,Ce,YZv),(Nu,Ce,YZw),( Nu,Ce,YZx),(Nu,Ce,YZy),(Nu,Ce,YZz),(Nu,Ce,YZaa),(Nu,Ce,YZab),(Nu,Ce,YZac),(Nu,Ce, YZad),(Nu,Ce,YZae),(Nu,Ce,YZaf),(Nu,Ce,YZag),(Nu,Cf,YZa),(Nu,Cf,YZb),(Nu,Cf,YZc),( Nu,Cf,YZd),(Nu,Cf,YZe),(Nu,Cf,YZf),(Nu,Cf,YZg),(Nu,Cf,YZh),(Nu,Cf,YZi),(Nu,Cf,YZj), (Nu,Cf,YZk),(Nu,Cf,YZl),(Nu,Cf,YZm),(Nu,Cf,YZn),(Nu,Cf,YZo),(Nu,Cf,YZp),(Nu,Cf,YZ q),(Nu,Cf,YZr),(Nu,Cf,YZs),(Nu,Cf,YZt),(Nu,Cf,YZu),(Nu,Cf,YZv),(Nu,Cf,YZw),(Nu,Cf,Y Zx),(Nu,Cf,YZy),(Nu,Cf,YZz),(Nu,Cf,YZaa),(Nu,Cf,YZab),(Nu,Cf,YZac),(Nu,Cf,YZad),(N u,Cf,YZae),(Nu,Cf,YZaf),(Nu,Cf,YZag),(Nu,Cg,YZa),(Nu,Cg,YZb),(Nu,Cg,YZc),(Nu,Cg,Y Zd),(Nu,Cg,YZe),(Nu,Cg,YZf),(Nu,Cg,YZg),(Nu,Cg,YZh),(Nu,Cg,YZi),(Nu,Cg,YZj),(Nu,C g,YZk),(Nu,Cg,YZl),(Nu,Cg,YZm),(Nu,Cg,YZn),(Nu,Cg,YZo),(Nu,Cg,YZp),(Nu,Cg,YZq),( Nu,Cg,YZr),(Nu,Cg,YZs),(Nu,Cg,YZt),(Nu,Cg,YZu),(Nu,Cg,YZv),(Nu,Cg,YZw),(Nu,Cg,Y Zx),(Nu,Cg,YZy),(Nu,Cg,YZz),(Nu,Cg,YZaa),(Nu,Cg,YZab),(Nu,Cg,YZac),(Nu,Cg,YZad),( Nu,Cg,YZae),(Nu,Cg,YZaf),(Nu,Cg,YZag),(Nu,Ch,YZa),(Nu,Ch,YZb),(Nu,Ch,YZc),(Nu,Ch ,YZd),(Nu,Ch,YZe),(Nu,Ch,YZf),(Nu,Ch,YZg),(Nu,Ch,YZh),(Nu,Ch,YZi),(Nu,Ch,YZj),(Nu ,Ch,YZk),(Nu,Ch,YZl),(Nu,Ch,YZm),(Nu,Ch,YZn),(Nu,Ch,YZo),(Nu,Ch,YZp),(Nu,Ch,YZq) ,(Nu,Ch,YZr),(Nu,Ch,YZs),(Nu,Ch,YZt),(Nu,Ch,YZu),(Nu,Ch,YZv),(Nu,Ch,YZw),(Nu,Ch, YZx),(Nu,Ch,YZy),(Nu,Ch,YZz),(Nu,Ch,YZaa),(Nu,Ch,YZab),(Nu,Ch,YZac),(Nu,Ch,YZad) ,(Nu,Ch,YZae),(Nu,Ch,YZaf),(Nu,Ch,YZag),(Nu,Ci,YZa),(Nu,Ci,YZb),(Nu,Ci,YZc),(Nu,Ci, YZd),(Nu,Ci,YZe),(Nu,Ci,YZf),(Nu,Ci,YZg),(Nu,Ci,YZh),(Nu,Ci,YZi),(Nu,Ci,YZj),(Nu,Ci, YZk),(Nu,Ci,YZl),(Nu,Ci,YZm),(Nu,Ci,YZn),(Nu,Ci,YZo),(Nu,Ci,YZp),(Nu,Ci,YZq),(Nu,C i,YZr),(Nu,Ci,YZs),(Nu,Ci,YZt),(Nu,Ci,YZu),(Nu,Ci,YZv),(Nu,Ci,YZw),(Nu,Ci,YZx),(Nu, Ci,YZy),(Nu,Ci,YZz),(Nu,Ci,YZaa),(Nu,Ci,YZab),(Nu,Ci,YZac),(Nu,Ci,YZad),(Nu,Ci,YZae) ,(Nu,Ci,YZaf),(Nu,Ci,YZag),(Nu,Cj,YZa),(Nu,Cj,YZb),(Nu,Cj,YZc),(Nu,Cj,YZd),(Nu,Cj,Y Ze),(Nu,Cj,YZf),(Nu,Cj,YZg),(Nu,Cj,YZh),(Nu,Cj,YZi),(Nu,Cj,YZj),(Nu,Cj,YZk),(Nu,Cj,Y Zl),(Nu,Cj,YZm),(Nu,Cj,YZn),(Nu,Cj,YZo),(Nu,Cj,YZp),(Nu,Cj,YZq),(Nu,Cj,YZr),(Nu,Cj, YZs),(Nu,Cj,YZt),(Nu,Cj,YZu),(Nu,Cj,YZv),(Nu,Cj,YZw),(Nu,Cj,YZx),(Nu,Cj,YZy),(Nu,Cj ,YZz),(Nu,Cj,YZaa),(Nu,Cj,YZab),(Nu,Cj,YZac),(Nu,Cj,YZad),(Nu,Cj,YZae),(Nu,Cj,YZaf), (Nu,Cj,YZag),(Nv,Ca,YZa),(Nv,Ca,YZb),(Nv,Ca,YZc),(Nv,Ca,YZd),(Nv,Ca,YZe),(Nv,Ca,Y Zf),(Nv,Ca,YZg),(Nv,Ca,YZh),(Nv,Ca,YZi),(Nv,Ca,YZj),(Nv,Ca,YZk),(Nv,Ca,YZl),(Nv,Ca, YZm),(Nv,Ca,YZn),(Nv,Ca,YZo),(Nv,Ca,YZp),(Nv,Ca,YZq),(Nv,Ca,YZr),(Nv,Ca,YZs),(Nv ,Ca,YZt),(Nv,Ca,YZu),(Nv,Ca,YZv),(Nv,Ca,YZw),(Nv,Ca,YZx),(Nv,Ca,YZy),(Nv,Ca,YZz), (Nv,Ca,YZaa),(Nv,Ca,YZab),(Nv,Ca,YZac),(Nv,Ca,YZad),(Nv,Ca,YZae),(Nv,Ca,YZaf),(Nv ,Ca,YZag),(Nv,Cb,YZa),(Nv,Cb,YZb),(Nv,Cb,YZc),(Nv,Cb,YZd),(Nv,Cb,YZe),(Nv,Cb,YZf) ,(Nv,Cb,YZg),(Nv,Cb,YZh),(Nv,Cb,YZi),(Nv,Cb,YZj),(Nv,Cb,YZk),(Nv,Cb,YZl),(Nv,Cb, YZm),(Nv,Cb,YZn),(Nv,Cb,YZo),(Nv,Cb,YZp),(Nv,Cb,YZq),(Nv,Cb,YZr),(Nv,Cb,YZs),(N v,Cb,YZt),(Nv,Cb,YZu),(Nv,Cb,YZv),(Nv,Cb,YZw),(Nv,Cb,YZx),(Nv,Cb,YZy),(Nv,Cb,YZ z),(Nv,Cb,YZaa),(Nv,Cb,YZab),(Nv,Cb,YZac),(Nv,Cb,YZad),(Nv,Cb,YZae),(Nv,Cb,YZaf),( Nv,Cb,YZag),(Nv,Cc,YZa),(Nv,Cc,YZb),(Nv,Cc,YZc),(Nv,Cc,YZd),(Nv,Cc,YZe),(Nv,Cc,Y Zf),(Nv,Cc,YZg),(Nv,Cc,YZh),(Nv,Cc,YZi),(Nv,Cc,YZj),(Nv,Cc,YZk),(Nv,Cc,YZl),(Nv,Cc, YZm),(Nv,Cc,YZn),(Nv,Cc,YZo),(Nv,Cc,YZp),(Nv,Cc,YZq),(Nv,Cc,YZr),(Nv,Cc,YZs),(Nv ,Cc,YZt),(Nv,Cc,YZu),(Nv,Cc,YZv),(Nv,Cc,YZw),(Nv,Cc,YZx),(Nv,Cc,YZy),(Nv,Cc,YZz), (Nv,Cc,YZaa),(Nv,Cc,YZab),(Nv,Cc,YZac),(Nv,Cc,YZad),(Nv,Cc,YZae),(Nv,Cc,YZaf),(Nv ,Cc,YZag),(Nv,Cd,YZa),(Nv,Cd,YZb),(Nv,Cd,YZc),(Nv,Cd,YZd),(Nv,Cd,YZe),(Nv,Cd,YZf) ,(Nv,Cd,YZg),(Nv,Cd,Ya),(Nv,Cd,YZi),(Nv,Cd,YZj),(Nv,Cd,YZk),(Nv,Cd,YZl),(Nv,Cd, YZm),(Nv,Cd,YZn),(Nv,Cd,YZo),(Nv,Cd,YZp),(Nv,Cd,YZq),(Nv,Cd,YZr),(Nv,Cd,YZs),(N v,Cd,YZt),(Nv,Cd,YZu),(Nv,Cd,YZv),(Nv,Cd,YZw),(Nv,Cd,YZx),(Nv,Cd,YZy),(Nv,Cd,YZ z),(Nv,Cd,YZaa),(Nv,Cd,YZab),(Nv,Cd,YZac),(Nv,Cd,YZad),(Nv,Cd,YZae),(Nv,Cd,YZaf),( Nv,Cd,YZag),(Nv,Ce,YZa),(Nv,Ce,YZb),(Nv,Ce,YZc),(Nv,Ce,YZd),(Nv,Ce,YZe),(Nv,Ce,Y Zf),(Nv,Ce,YZg),(Nv,Ce,YZh),(Nv,Ce,YZi),(Nv,Ce,YZj),(Nv,Ce,YZk),(Nv,Ce,YZl),(Nv,Ce, YZm),(Nv,Ce,YZn),(Nv,Ce,YZo),(Nv,Ce,YZp),(Nv,Ce,YZq),(Nv,Ce,YZr),(Nv,Ce,YZs),(Nv ,Ce,YZt),(Nv,Ce,YZu),(Nv,Ce,YZv),(Nv,Ce,YZw),(Nv,Ce,YZx),(Nv,Ce,YZy),(Nv,Ce,YZz), (Nv,Ce,YZaa),(Nv,Ce,YZab),(Nv,Ce,YZac),(Nv,Ce,YZad),(Nv,Ce,YZae),(Nv,Ce,YZaf),(Nv ,Ce,YZag),(Nv,Cf,YZa),(Nv,Cf,YZb),(Nv,Cf,YZc),(Nv,Cf,YZd),(Nv,Cf,YZe),(Nv,Cf,YZf),( Nv,Cf,YZg),(Nv,Cf,YZh),(Nv,Cf,YZi),(Nv,Cf,YZj),(Nv,Cf,YZk),(Nv,Cf,YZl),(Nv,Cf,YZm), (Nv,Cf,YZn),(Nv,Cf,YZo),(Nv,Cf,YZp),(Nv,Cf,YZq),(Nv,Cf,YZr),(Nv,Cf,YZs),(Nv,Cf,YZt) ,(Nv,Cf,YZu),(Nv,Cf,YZv),(Nv,Cf,YZw),(Nv,Cf,YZx),(Nv,Cf,YZy),(Nv,Cf,YZz),(Nv,Cf,Y Zaa),(Nv,Cf,YZab),(Nv,Cf,YZac),(Nv,Cf,YZad),(Nv,Cf,YZae),(Nv,Cf,YZaf),(Nv,Cf,YZag), (Nv,Cg,YZa),(Nv,Cg,YZb),(Nv,Cg,YZc),(Nv,Cg,YZd),(Nv,Cg,YZe),(Nv,Cg,YZf),(Nv,Cg,Y Zg),(Nv,Cg,YZh),(Nv,Cg,YZi),(Nv,Cg,YZj),(Nv,Cg,YZk),(Nv,Cg,YZl),(Nv,Cg,YZm),(Nv,C g,YZn),(Nv,Cg,YZo),(Nv,Cg,YZp),(Nv,Cg,YZq),(Nv,Cg,YZr),(Nv,Cg,YZs),(Nv,Cg,YZt),(N v,Cg,YZu),(Nv,Cg,YZv),(Nv,Cg,YZw),(Nv,Cg,YZx),(Nv,Cg,YZy),(Nv,Cg,YZz),(Nv,Cg,YZ aa),(Nv,Cg,YZab),(Nv,Cg,YZac),(Nv,Cg,YZad),(Nv,Cg,YZae),(Nv,Cg,YZaf),(Nv,Cg,YZag) ,(Nv,Ch,YZa),(Nv,Ch,YZb),(Nv,Ch,YZc),(Nv,Ch,YZd),(Nv,Ch,YZe),(Nv,Ch,YZf),(Nv,Ch, YZg),(Nv,Ch,YZh),(Nv,Ch,YZi),(Nv,Ch,YZj),(Nv,Ch,YZk),(Nv,Ch,YZl),(Nv,Ch,YZm),(Nv ,Ch,YZn),(Nv,Ch,YZo),(Nv,Ch,YZp),(Nv,Ch,YZq),(Nv,Ch,YZr),(Nv,Ch,YZs),(Nv,Ch,YZt), (Nv,Ch,YZu),(Nv,Ch,YZv),(Nv,Ch,YZw),(Nv,Ch,YZx),(Nv,Ch,YZy),(Nv,Ch,YZz),(Nv,Ch, YZaa),(Nv,Ch,YZab),(Nv,Ch,YZac),(Nv,Ch,YZad),(Nv,Ch,YZae),(Nv,Ch,YZaf),(Nv,Ch,YZ ag),(Nv,Ci,YZa),(Nv,Ci,YZb),(Nv,Ci,YZc),(Nv,Ci,YZd),(Nv,Ci,YZe),(Nv,Ci,YZf),(Nv,Ci,Y Zg),(Nv,Ci,YZh),(Nv,Ci,YZi),(Nv,Ci,YZj),(Nv,Ci,YZic),(Nv,Ci,YZl),(Nv,Ci,YZm),(Nv,Ci,Y Zn),(Nv,Ci,YZo),(Nv,Ci,YZp),(Nv,Ci,YZq),(Nv,Ci,YZr),(Nv,Ci,YZs),(Nv,Ci,YZt),(Nv,Ci,Y Zu),(Nv,Ci,YZv),(Nv,Ci,YZw),(Nv,Ci,YZx),(Nv,Ci,YZy),(Nv,Ci,YZz),(Nv,Ci,YZaa),(Nv,Ci ,YZab),(Nv,Ci,YZac),(Nv,Ci,YZad),(Nv,Ci,YZae),(Nv,Ci,YZaf),(Nv,Ci,YZag),(Nv,Cj,YZa), (Nv,Cj,YZb),(Nv,Cj,YZc),(Nv,Cj,YZd),(Nv,Cj,YZe),(Nv,Cj,YZf),(Nv,Cj,YZg),(Nv,Cj,YZh) ,(Nv,Cj,YZi),(Nv,Cj,YZj),(Nv,Cj,YZk),(Nv,Cj,YZl),(Nv,Cj,YZm),(Nv,Cj,YZn),(Nv,Cj,YZo) ,(Nv,Cj,YZp),(Nv,Cj,YZq),(Nv,Cj,YZr),(Nv,Cj,YZs),(Nv,Cj,YZt),(Nv,Cj,YZu),(Nv,Cj,YZv) ,(Nv,Cj,YZw),(Nv,Cj,YZx),(Nv,Cj,YZy),(Nv,Cj,YZz),(Nv,Cj,YZaa),(Nv,Cj,YZab),(Nv,Cj,Y Zac),(Nv,Cj,YZad),(Nv,Cj,YZae),(Nv,Cj,YZaf),(Nv,Cj,YZag),(Nw,Ca,YZa),(Nw,Ca,YZb),( Nw,Ca,YZc),(Nw,Ca,YZd),(Nw,Ca,YZe),(Nw,Ca,YZf),(Nw,Ca,YZg),(Nw,Ca,YZh),(Nw,Ca, YZi),(Nw,Ca,YZj),(Nw,Ca,YZk),(Nw,Ca,YZl),(Nw,Ca,YZm),(Nw,Ca,YZn),(Nw,Ca,YZo),( Nw,Ca,YZp),(Nw,Ca,YZq),(Nw,Ca,YZr),(Nw,Ca,YZs),(Nw,Ca,YZt),(Nw,Ca,YZu),(Nw,Ca, YZv),(Nw,Ca,YZw),(Nw,Ca,YZx),(Nw,Ca,YZy),(Nw,Ca,YZz),(Nw,Ca,YZaa),(Nw,Ca,YZa b),(Nw,Ca,YZac),(Nw,Ca,YZad),(Nw,Ca,YZae),(Nw,Ca,YZaf),(Nw,Ca,YZag),(Nw,Cb,YZa) ,(Nw,Cb,YZb),(Nw,Cb,YZc),(Nw,Cb,YZd),(Nw,Cb,YZe),(Nw,Cb,YZf),(Nw,Cb,YZg),(Nw, Cb,YZh),(Nw,Cb,YZi),(Nw,Cb,YZj),(Nw,Cb,YZk),(Nw,Cb,YZl),(Nw,Cb,YZm),(Nw,Cb,YZ n),(Nw,Cb,YZo),(Nw,Cb,YZp),(Nw,Cb,YZq),(Nw,Cb,YZr),(Nw,Cb,YZs),(Nw,Cb,YZt),(Nw ,Cb,YZu),(Nw,Cb,YZv),(Nw,Cb,YZw),(Nw,Cb,YZx),(Nw,Cb,YZy),(Nw,Cb,YZz),(Nw,Cb, YZaa),(Nw,Cb,YZab),(Nw,Cb,YZac),(Nw,Cb,YZad),(Nw,Cb,YZae),(Nw,Cb,YZaf),(Nw,Cb, YZag),(Nw,Cc,YZa),(Nw,Cc,YZb),(Nw,Cc,YZc),(Nw,Cc,YZd),(Nw,Cc,YZe),(Nw,Cc,YZf), (Nw,Cc,YZg),(Nw,Cc,YZh),(Nw,Cc,YZi),(Nw,Cc,YZj),(Nw,Cc,YZk),(Nw,Cc,YZl),(Nw,Cc, YZm),(Nw,Cc,YZn),(Nw,Cc,YZo),(Nw,Cc,YZp),(Nw,Cc,YZq),(Nw,Cc,YZr),(Nw,Cc,YZs),( Nw,Cc,YZt),(Nw,Cc,YZu),(Nw,Cc,YZv),(Nw,Cc,YZw),(Nw,Cc,YZx),(Nw,Cc,YZy),(Nw,C c,YZz),(Nw,Cc,YZaa),(Nw,Cc,YZab),(Nw,Cc,YZac),(Nw,Cc,YZad),(Nw,Cc,YZae),(Nw,Cc, YZaf),(Nw,Cc,YZag),(Nw,Cd,YZa),(Nw,Cd,YZb),(Nw,Cd,YZc),(Nw,Cd,YZd),(Nw,Cd,YZe) ,(Nw,Cd,Ya),(Nw,Cd,YZg),(Nw,Cd,Ya),(Nw,Cd,YZi),(Nw,Cd,YZj),(Nw,Cd,YZk),(Nw, Cd,YZl),(Nw,Cd,YZm),(Nw,Cd,YZn),(Nw,Cd,YZo),(Nw,Cd,YZp),(Nw,Cd,YZq),(Nw,Cd,Y Zr),(Nw,Cd,YZs),(Nw,Cd,YZt),(Nw,Cd,YZu),(Nw,Cd,YZv),(Nw,Cd,YZw),(Nw,Cd,YZx),( Nw,Cd,YZy),(Nw,Cd,YZz),(Nw,Cd,YZaa),(Nw,Cd,YZab),(Nw,Cd,YZac),(Nw,Cd,YZad),(N w,Cd,YZae),(Nw,Cd,YZaf),(Nw,Cd,YZag),(Nw,Ce,YZa),(Nw,Ce,YZb),(Nw,Ce,YZc),(Nw, Ce,YZd),(Nw,Ce,YZe),(Nw,Ce,YZf),(Nw,Ce,YZg),(Nw,Ce,YZh),(Nw,Ce,YZi),(Nw,Ce,YZj) ,(Nw,Ce,YZk),(Nw,Ce,YZl),(Nw,Ce,YZm),(Nw,Ce,YZn),(Nw,Ce,YZo),(Nw,Ce,YZp),(Nw, Ce,YZq),(Nw,Ce,YZr),(Nw,Ce,YZs),(Nw,Ce,YZt),(Nw,Ce,YZu),(Nw,Ce,YZv),(Nw,Ce,YZ w),(Nw,Ce,YZx),(Nw,Ce,YZy),(Nw,Ce,YZz),(Nw,Ce,YZaa),(Nw,Ce,YZab),(Nw,Ce,YZac), (Nw,Ce,YZad),(Nw,Ce,YZae),(Nw,Ce,YZaf),(Nw,Ce,YZag),(Nw,Cf,YZa),(Nw,Cf,YZb),(N w,Cf,YZc),(Nw,Cf,YZd),(Nw,Cf,YZe),(Nw,Cf,YZf),(Nw,Cf,YZg),(Nw,Cf,YZh),(Nw,Cf,YZ i),(Nw,Cf,YZj),(Nw,Cf,YZk),(Nw,Cf,YZl),(Nw,Cf,YZm),(Nw,Cf,YZn),(Nw,Cf,YZo),(Nw,C f,YZp),(Nw,Cf,YZq),(Nw,Cf,YZr),(Nw,Cf,YZs),(Nw,Cf,YZt),(Nw,Cf,YZu),(Nw,Cf,YZy),( Nw,Cf,YZw),(Nw,Cf,YZx),(Nw,Cf,YZy),(Nw,Cf,YZz),(Nw,Cf,YZaa),(Nw,Cf,YZab),(Nw,C f,YZac),(Nw,Cf,YZad),(Nw,Cf,YZae),(Nw,Cf,YZaf),(Nw,Cf,YZag),(Nw,Cg,YZa),(Nw,Cg, YZb),(Nw,Cg,YZc),(Nw,Cg,YZd),(Nw,Cg,YZe),(Nw,Cg,YZf),(Nw,Cg,YZg),(Nw,Cg,YZh), (Nw,Cg,YZi),(Nw,Cg,YZj),(Nw,Cg,YZk),(Nw,Cg,YZl),(Nw,Cg,YZm),(Nw,Cg,YZn),(Nw,C g,YZo),(Nw,Cg,YZp),(Nw,Cg,YZq),(Nw,Cg,YZr),(Nw,Cg,YZs),(Nw,Cg,YZt),(Nw,Cg,YZu) ,(Nw,Cg,YZy),(Nw,Cg,YZw),(Nw,Cg,YZx),(Nw,Cg,YZy),(Nw,Cg,YZz),(Nw,Cg,YZaa),(N w,Cg,YZab),(Nw,Cg,YZac),(Nw,Cg,YZad),(Nw,Cg,YZae),(Nw,Cg,YZaf),(Nw,Cg,YZag),(N w,Ch,YZa),(Nw,Ch,YZb),(Nw,Ch,YZc),(Nw,Ch,YZd),(Nw,Ch,YZe),(Nw,Ch,YZf),(Nw,Ch, YZg),(Nw,Ch,YZh),(Nw,Ch,YZi),(Nw,Ch,YZj),(Nw,Ch,Ya),(Nw,Ch,YZl),(Nw,Ch,YZm),( Nw,Ch,YZn),(Nw,Ch,YZo),(Nw,Ch,YZp),(Nw,Ch,YZq),(Nw,Ch,YZr),(Nw,Ch,YZs),(Nw,C h,YZt),(Nw,Ch,YZu),(Nw,Ch,YZy),(Nw,Ch,YZw),(Nw,Ch,YZx),(Nw,Ch,YZy),(Nw,Ch,YZ z),(Nw,Ch,YZaa),(Nw,Ch,YZab),(Nw,Ch,YZac),(Nw,Ch,YZad),(Nw,Ch,YZae),(Nw,Ch,YZa f),(Nw,Ch,YZag),(Nw,Ci,YZa),(Nw,Ci,YZb),(Nw,Ci,YZc),(Nw,Ci,Yal),(Nw,Ci,YZe),(Nw, Ci,YZf),(Nw,Ci,YZg),(Nw,Ci,YZh),(Nw,Ci,YZi),(Nw,Ci,YZj),(Nw,Ci,YZk),(Nw,Ci,YZl),( Nw,Ci,YZm),(Nw,Ci,YZn),(Nw,Ci,YZo),(Nw,Ci,YZp),(Nw,Ci,YZq),(Nw,Ci,YZr),(Nw,Ci,Y Zs),(Nw,Ci,YZt),(Nw,Ci,YZu),(Nw,Ci,YZy),(Nw,Ci,YZw),(Nw,Ci,YZx),(Nw,Ci,YZy),(Nw, Ci,YZz),(Nw,Ci,YZaa),(Nw,Ci,YZab),(Nw,Ci,YZac),(Nw,Ci,YZad),(Nw,Ci,YZae),(Nw,Ci, YZaf),(Nw,Ci,YZag),(Nw,Cj,YZa),(Nw,Cj,YZb),(Nw,Cj,YZc),(Nw,Cj,YZd),(Nw,Cj,YZe),( Nw,Cj,YZf),(Nw,Cj,YZg),(Nw,Cj,YZh),(Nw,Cj,YZi),(Nw,Cj,YZj),(Nw,Cj,YZk),(Nw,Cj,YZ l),(Nw,Cj,YZm),(Nw,Cj,YZn),(Nw,Cj,YZo),(Nw,Cj,YZp),(Nw,Cj,YZq),(Nw,Cj,YZr),(Nw,C j,YZs),(Nw,Cj,YZt),(Nw,Cj,YZu),(Nw,Cj,YZv),(Nw,Cj,YZw),(Nw,Cj,YZx),(Nw,Cj,YZy),( Nw,Cj,YZz),(Nw,Cj,YZaa),(Nw,Cj,YZab),(Nw,Cj,YZac),(Nw,Cj,YZad),(Nw,Cj,YZae),(Nw, Cj,YZaf),(Nw,Cj,YZag),(Nx,Ca,YZa),(Nx,Ca,YZb),(Nx,Ca,YZc),(Nx,Ca,YZd),(Nx,Ca,YZe) ,(Nx,Ca,YZf),(Nx,Ca,YZg),(Nx,Ca,YZh),(Nx,Ca,YZi),(Nx,Ca,YZj),(Nx,Ca,YZk),(Nx,Ca,Y Zl),(Nx,Ca,YZm),(Nx,Ca,YZn),(Nx,Ca,YZo),(Nx,Ca,YZp),(Nx,Ca,YZq),(Nx,Ca,YZr),(Nx,C a,YZs),(Nx,Ca,YZt),(Nx,Ca,YZu),(Nx,Ca,YZy),(Nx,Ca,YZw),(Nx,Ca,YZx),(Nx,Ca,YZy),(N x,Ca,YZz),(Nx,Ca,YZaa),(Nx,Ca,YZab),(Nx,Ca,YZac),(Nx,Ca,YZad),(Nx,Ca,YZae),(Nx,Ca, YZaf),(Nx,Ca,YZag),(Nx,Cb,YZa),(Nx,Cb,YZb),(Nx,Cb,YZc),(Nx,Cb,YZd),(Nx,Cb,YZe),( Nx,Cb,YZf),(Nx,Cb,YZg),(Nx,Cb,YZh),(Nx,Cb,YZi),(Nx,Cb,YZj),(Nx,Cb,YZk),(Nx,Cb,YZ l),(Nx,Cb,YZm),(Nx,Cb,YZn),(Nx,Cb,YZo),(Nx,Cb,YZp),(Nx,Cb,YZq),(Nx,Cb,YZr),(Nx,C b,YZs),(Nx,Cb,YZt),(Nx,Cb,YZu),(Nx,Cb,YZy),(Nx,Cb,YZw),(Nx,Cb,YZx),(Nx,Cb,YZy),( Nx,Cb,YZz),(Nx,Cb,YZaa),(Nx,Cb,YZab),(Nx,Cb,YZac),(Nx,Cb,YZad),(Nx,Cb,YZae),(Nx, Cb,YZaf),(Nx,Cb,YZag),(Nx,Cc,YZa),(Nx,Cc,YZb),(Nx,Cc,YZc),(Nx,Cc,YZd),(Nx,Cc,YZe) ,(Nx,Cc,YZf),(Nx,Cc,YZg),(Nx,Cc,YZh),(Nx,Cc,YZi),(Nx,Cc,YZj),(Nx,Cc,YZk),(Nx,Cc,Y Zl),(Nx,Cc,YZm),(Nx,Cc,YZn),(Nx,Cc,YZo),(Nx,Cc,YZp),(Nx,Cc,YZq),(Nx,Cc,YZr),(Nx,C c,YZs),(Nx,Cc,YZt),(Nx,Cc,YZu),(Nx,Cc,YZy),(Nx,Cc,YZw),(Nx,Cc,YZx),(Nx,Cc,YZy),(N x,Cc,YZz),(Nx,Cc,YZaa),(Nx,Cc,YZab),(Nx,Cc,YZac),(Nx,Cc,YZad),(Nx,Cc,YZae),(Nx,Cc, YZaf),(Nx,Cc,YZag),(Nx,Cd,YZa),(Nx,Cd,YZb),(Nx,Cd,YZc),(Nx,Cd,YZd),(Nx,Cd,YZe),( Nx,Cd,YZf),(Nx,Cd,YZg),(Nx,Cd,YZh),(Nx,Cd,YZi),(Nx,Cd,YZj),(Nx,Cd,YZk),(Nx,Cd,YZ l),(Nx,Cd,YZm),(Nx,Cd,YZn),(Nx,Cd,YZo),(Nx,Cd,YZp),(Nx,Cd,YZq),(Nx,Cd,YZr),(Nx,C d,YZs),(Nx,Cd,YZt),(Nx,Cd,YZu),(Nx,Cd,YZy),(Nx,Cd,YZw),(Nx,Cd,YZx),(Nx,Cd,YZy),( Nx,Cd,YZz),(Nx,Cd,YZaa),(Nx,Cd,YZab),(Nx,Cd,YZac),(Nx,Cd,YZad),(Nx,Cd,YZae),(Nx, Cd,YZaf),(Nx,Cd,YZag),(Nx,Ce,YZa),(Nx,Ce,YZb),(Nx,Ce,YZc),(Nx,Ce,YZd),(Nx,Ce,YZe) ,(Nx,Ce,YZf),(Nx,Ce,YZg),(Nx,Ce,YZh),(Nx,Ce,YZi),(Nx,Ce,YZj),(Nx,Ce,YZk),(Nx,Ce,Y Zl),(Nx,Ce,YZm),(Nx,Ce,YZn),(Nx,Ce,YZo),(Nx,Ce,YZp),(Nx,Ce,YZq),(Nx,Ce,YZr),(Nx,C e,YZs),(Nx,Ce,YZt),(Nx,Ce,YZu),(Nx,Ce,YZy),(Nx,Ce,YZw),(Nx,Ce,YZx),(Nx,Ce,YZy),(N x,Ce,YZz),(Nx,Ce,YZaa),(Nx,Ce,YZab),(Nx,Ce,YZac),(Nx,Ce,YZad),(Nx,Ce,YZae),(Nx,Ce, YZaf),(Nx,Ce,YZag),(Nx,Cf,YZa),(Nx,Cf,YZb),(Nx,Cf,YZc),(Nx,Cf,YZd),(Nx,Cf,YZe),(Nx ,Cf,YZf),(Nx,Cf,YZg),(Nx,Cf,YZh),(Nx,Cf,YZi),(Nx,Cf,YZj),(Nx,Cf,YZk),(Nx,Cf,YZl),(Nx ,Cf,YZm),(Nx,Cf,YZn),(Nx,Cf,YZo),(Nx,Cf,YZp),(Nx,Cf,YZq),(Nx,Cf,YZr),(Nx,Cf,YZs),( Nx,Cf,YZt),(Nx,Cf,YZu),(Nx,Cf,YZy),(Nx,Cf,YZw),(Nx,Cf,YZx),(Nx,Cf,YZy),(Nx,Cf,YZz) ,(Nx,Cf,YZaa),(Nx,Cf,YZab),(Nx,Cf,YZac),(Nx,Cf,YZad),(Nx,Cf,YZae),(Nx,Cf,YZaf),(Nx, Cf,YZag),(Nx,Cg,YZa),(Nx,Cg,YZb),(Nx,Cg,YZc),(Nx,Cg,YZd),(Nx,Cg,YZe),(Nx,Cg,YZf), (Nx,Cg,YZg),(Nx,Cg,YZh),(Nx,Cg,YZi),(Nx,Cg,YZj),(Nx,Cg,YZk),(Nx,Cg,YZl),(Nx,Cg,Y Zm),(Nx,Cg,YZn),(Nx,Cg,YZo),(Nx,Cg,YZp),(Nx,Cg,YZq),(Nx,Cg,YZr),(Nx,Cg,YZs),(Nx, Cg,YZt),(Nx,Cg,YZu),(Nx,Cg,YZy),(Nx,Cg,YZw),(Nx,Cg,YZx),(Nx,Cg,YZy),(Nx,Cg,YZz), (Nx,Cg,YZaa),(Nx,Cg,YZab),(Nx,Cg,YZac),(Nx,Cg,YZad),(Nx,Cg,YZae),(Nx,Cg,YZaf),(N x,Cg,YZag),(Nx,Ch,YZa),(Nx,Ch,YZb),(Nx,Ch,YZc),(Nx,Ch,YZd),(Nx,Ch,YZe),(Nx,Ch,YZ f),(Nx,Ch,YZg),(Nx,Ch,YZh),(Nx,Ch,YZi),(Nx,Ch,YZj),(Nx,Ch,YZk),(Nx,Ch,YZl),(Nx,Ch, YZm),(Nx,Ch,YZn),(Nx,Ch,YZo),(Nx,Ch,YZp),(Nx,Ch,YZq),(Nx,Ch,YZr),(Nx,Ch,YZs),(N x,Ch,YZt),(Nx,Ch,YZu),(Nx,Ch,YZv),(Nx,Ch,YZw),(Nx,Ch,YZx),(Nx,Ch,YZy),(Nx,Ch,YZ z),(Nx,Ch,YZaa),(Nx,Ch,YZab),(Nx,Ch,YZac),(Nx,Ch,YZad),(Nx,Ch,YZae),(Nx,Ch,YZaf),( Nx,Ch,YZag),(Nx,Ci,YZa),(Nx,Ci,YZb),(Nx,Ci,YZc),(Nx,Ci,YZd),(Nx,Ci,YZe),(Nx,Ci,YZf) ,(Nx,Ci,YZg),(Nx,Ci,YZh),(Nx,Ci,YZi),(Nx,Ci,YZj),(Nx,Ci,YZk),(Nx,Ci,YZl),(Nx,Ci,YZm) ,(Nx,Ci,YZn),(Nx,Ci,YZo),(Nx,Ci,YZp),(Nx,Ci,YZq),(Nx,Ci,YZr),(Nx,Ci,YZs),(Nx,Ci,YZt) ,(Nx,Ci,YZu),(Nx,Ci,YZv),(Nx,Ci,YZw),(Nx,Ci,YZx),(Nx,Ci,YZy),(Nx,Ci,YZz),(Nx,Ci,YZ aa),(Nx,Ci,YZab),(Nx,Ci,YZac),(Nx,Ci,YZad),(Nx,Ci,YZae),(Nx,Ci,YZaf),(Nx,Ci,YZag),(N x,Cj,YZa),(Nx,Cj,YZb),(Nx,Cj,YZc),(Nx,Cj,YZd),(Nx,Cj,YZe),(Nx,Cj,YZf),(Nx,Cj,YZg),(N x,Cj,YZh),(Nx,Cj,YZi),(Nx,Cj,YZj),(Nx,Cj,YZk),(Nx,Cj,YZl),(Nx,Cj,YZm),(Nx,Cj,YZn),(N x,Cj,YZo),(Nx,Cj,YZp),(Nx,Cj,YZq),(Nx,Cj,YZr),(Nx,Cj,YZs),(Nx,Cj,YZt),(Nx,Cj,YZu),(N x,Cj,YZv),(Nx,Cj,YZw),(Nx,Cj,YZx),(Nx,Cj,YZy),(Nx,Cj,YZz),(Nx,Cj,YZaa),(Nx,Cj,YZab) ,(Nx,Cj,YZac),(Nx,Cj,YZad),(Nx,Cj,YZae),(Nx,Cj,YZaf),(Nx,Cj,YZag),(Ny,Ca,YZa),(Ny,C a,YZb),(Ny,Ca,YZc),(Ny,Ca,YZd),(Ny,Ca,YZe),(Ny,Ca,YZf),(Ny,Ca,YZg),(Ny,Ca,YZh),(N y,Ca,YZi),(Ny,Ca,YZj),(Ny,Ca,YZk),(Ny,Ca,YZl),(Ny,Ca,YZm),(Ny,Ca,YZn),(Ny,Ca,YZo) ,(Ny,Ca,YZp),(Ny,Ca,YZq),(Ny,Ca,YZr),(Ny,Ca,YZs),(Ny,Ca,YZt),(Ny,Ca,YZu),(Ny,Ca,Y Zv),(Ny,Ca,YZw),(Ny,Ca,YZx),(Ny,Ca,YZy),(Ny,Ca,YZz),(Ny,Ca,YZaa),(Ny,Ca,YZab),(N y,Ca,YZac),(Ny,Ca,YZad),(Ny,Ca,YZae),(Ny,Ca,YZaf),(Ny,Ca,YZag),(Ny,Cb,YZa),(Ny,Cb, YZb),(Ny,Cb,YZc),(Ny,Cb,YZd),(Ny,Cb,YZe),(Ny,Cb,YZf),(Ny,Cb,YZg),(Ny,Cb,YZh),(Ny, ,Cb,YZi),(Ny,Cb,YZj),(Ny,Cb,YZk),(Ny,Cb,YZl),(Ny,Cb,YZm),(Ny,Cb,YZn),(Ny,Cb,YZo), (Ny,Cb,YZp),(Ny,Cb,YZq),(Ny,Cb,YZr),(Ny,Cb,YZs),(Ny,Cb,YZt),(Ny,Cb,YZu),(Ny,Cb,Y Zv),(Ny,Cb,YZw),(Ny,Cb,YZx),(Ny,Cb,YZy),(Ny,Cb,YZz),(Ny,Cb,YZaa),(Ny,Cb,YZab),(N y,Cb,YZac),(Ny,Cb,YZad),(Ny,Cb,YZae),(Ny,Cb,YZaf),(Ny,Cb,YZag),(Ny,Cc,YZa),(Ny,Cc ,YZb),(Ny,Cc,YZc),(Ny,Cc,YZd),(Ny,Cc,YZe),(Ny,Cc,YZf),(Ny,Cc,YZg),(Ny,Cc,YZh),(Ny ,Cc,YZi),(Ny,Cc,YZj),(Ny,Cc,YZk),(Ny,Cc,YZl),(Ny,Cc,YZm),(Ny,Cc,YZn),(Ny,Cc,YZo),( Ny,Cc,YZp),(Ny,Cc,YZq),(Ny,Cc,YZr),(Ny,Cc,YZs),(Ny,Cc,YZt),(Ny,Cc,YZu),(Ny,Cc,YZ v),(Ny,Cc,YZw),(Ny,Cc,YZx),(Ny,Cc,YZy),(Ny,Cc,YZz),(Ny,Cc,YZaa),(Ny,Cc,YZab),(Ny, Cc,YZac),(Ny,Cc,YZad),(Ny,Cc,YZae),(Ny,Cc,YZaf),(Ny,Cc,YZag),(Ny,Cd,YZa),(Ny,Cd,Y Zb),(Ny,Cd,YZc),(Ny,Cd,YZd),(Ny,Cd,YZe),(Ny,Cd,YZf),(Ny,Cd,YZg),(Ny,Cd,YZh),(Ny, Cd,YZi),(Ny,Cd,YZj),(Ny,Cd,YZl),(Ny,Cd,YZl),(Ny,Cd,YZm),(Ny,Cd,YZn),(Ny,Cd,YZo),( Ny,Cd,YZp),(Ny,Cd,YZq),(Ny,Cd,YZr),(Ny,Cd,YZs),(Ny,Cd,YZt),(Ny,Cd,YZu),(Ny,Cd,YZ v),(Ny,Cd,YZw),(Ny,Cd,YZx),(Ny,Cd,YZy),(Ny,Cd,YZz),(Ny,Cd,YZaa),(Ny,Cd,YZab),(Ny ,Cd,YZac),(Ny,Cd,YZad),(Ny,Cd,YZae),(Ny,Cd,YZaf),(Ny,Cd,YZag),(Ny,Ce,YZa),(Ny,Ce, YZb),(Ny,Ce,YZc),(Ny,Ce,YZd),(Ny,Ce,YZe),(Ny,Ce,YZf),(Ny,Ce,YZg),(Ny,Ce,YZh),(Ny, Ce,YZi),(Ny,Ce,YZj),(Ny,Ce,YZic),(Ny,Ce,YZl),(Ny,Ce,YZm),(Ny,Ce,YZn),(Ny,Ce,YZo),( Ny,Ce,YZp),(Ny,Ce,YZq),(Ny,Ce,YZr),(Ny,Ce,YZs),(Ny,Ce,YZt),(Ny,Ce,YZu),(Ny,Ce,YZ v),(Ny,Ce,YZw),(Ny,Ce,YZx),(Ny,Ce,YZy),(Ny,Ce,YZz),(Ny,Ce,YZaa),(Ny,Ce,YZab),(Ny, Ce,YZac),(Ny,Ce,YZad),(Ny,Ce,YZae),(Ny,Ce,YZaf),(Ny,Ce,YZag),(Ny,Cf,YZa),(Ny,Cf,Y Zb),(Ny,Cf,YZc),(Ny,Cf,YZd),(Ny,Cf,YZe),(Ny,Cf,YZf),(Ny,Cf,YZg),(Ny,Cf,YZh),(Ny,Cf, YZi),(Ny,Cf,YZj),(Ny,Cf,YZk),(Ny,Cf,YZl),(Ny,Cf,YZm),(Ny,Cf,YZn),(Ny,Cf,YZo),(Ny,C f,YZp),(Ny,Cf,YZq),(Ny,Cf,YZr),(Ny,Cf,YZs),(Ny,Cf,YZt),(Ny,Cf,YZu),(Ny,Cf,YZv),(Ny, Cf,YZw),(Ny,Cf,YZx),(Ny,Cf,YZy),(Ny,Cf,YZz),(Ny,Cf,YZaa),(Ny,Cf,YZab),(Ny,Cf,YZac) ,(Ny,Cf,YZad),(Ny,Cf,YZae),(Ny,Cf,YZaf),(Ny,Cf,YZag),(Ny,Cg,YZa),(Ny,Cg,YZb),(Ny, Cg,YZc),(Ny,Cg,YZd),(Ny,Cg,YZe),(Ny,Cg,YZf),(Ny,Cg,YZg),(Ny,Cg,YZh),(Ny,Cg,YZi),( Ny,Cg,YZj),(Ny,Cg,YZk),(Ny,Cg,YZl),(Ny,Cg,YZm),(Ny,Cg,YZn),(Ny,Cg,YZo),(Ny,Cg,Y Zp),(Ny,Cg,YZq),(Ny,Cg,YZr),(Ny,Cg,YZs),(Ny,Cg,YZt),(Ny,Cg,YZu),(Ny,Cg,YZv),(Ny,C g,YZw),(Ny,Cg,YZx),(Ny,Cg,YZy),(Ny,Cg,YZz),(Ny,Cg,YZaa),(Ny,Cg,YZab),(Ny,Cg,YZa c),(Ny,Cg,YZad),(Ny,Cg,YZae),(Ny,Cg,YZaf),(Ny,Cg,YZag),(Ny,Ch,YZa),(Ny,Ch,YZb),(N y,Ch,YZc),(Ny,Ch,YZd),(Ny,Ch,YZe),(Ny,Ch,YZf),(Ny,Ch,YZg),(Ny,Ch,YZh),(Ny,Ch,YZi) ,(Ny,Ch,YZj),(Ny,Ch,YZk),(Ny,Ch,YZl),(Ny,Ch,YZm),(Ny,Ch,YZn),(Ny,Ch,YZo),(Ny,Ch, YZp),(Ny,Ch,YZq),(Ny,Ch,YZr),(Ny,Ch,YZs),(Ny,Ch,YZt),(Ny,Ch,YZu),(Ny,Ch,YZv),(Ny, Ch,YZw),(Ny,Ch,YZx),(Ny,Ch,YZy),(Ny,Ch,YZz),(Ny,Ch,YZaa),(Ny,Ch,YZab),(Ny,Ch,YZ ac),(Ny,Ch,YZad),(Ny,Ch,YZae),(Ny,Ch,YZaf),(Ny,Ch,YZag),(Ny,Ci,YZa),(Ny,Ci,YZb),(N y,Ci,YZc),(Ny,Ci,YZd),(Ny,Ci,YZe),(Ny,Ci,YZf),(Ny,Ci,YZg),(Ny,Ci,YZh),(Ny,Ci,YZi),(N y,Ci,YZj),(Ny,Ci,YZk),(Ny,Ci,YZl),(Ny,Ci,YZm),(Ny,Ci,YZn),(Ny,Ci,YZo),(Ny,Ci,YZp),( Ny,Ci,YZq),(Ny,Ci,YZr),(Ny,Ci,YZs),(Ny,Ci,YZt),(Ny,Ci,YZu),(Ny,Ci,YZv),(Ny,Ci,YZw), (Ny,Ci,YZx),(Ny,Ci,YZy),(Ny,Ci,YZz),(Ny,Ci,YZaa),(Ny,Ci,YZab),(Ny,Ci,YZac),(Ny,Ci,Y Zad),(Ny,Ci,YZae),(Ny,Ci,YZaf),(Ny,Ci,YZag),(Ny,Cj,YZa),(Ny,Cj,YZb),(Ny,Cj,YZc),(Ny, Cj,YZd),(Ny,Cj,YZe),(Ny,Cj,YZf),(Ny,Cj,YZg),(Ny,Cj,YZh),(Ny,Cj,YZi),(Ny,Cj,YZj),(Ny, Cj,YZk),(Ny,Cj,YZl),(Ny,Cj,YZm),(Ny,Cj,YZn),(Ny,Cj,YZo),(Ny,Cj,YZp),(Ny,Cj,YZq),(N y,Cj,YZr),(Ny,Cj,YZs),(Ny,Cj,YZt),(Ny,Cj,YZu),(Ny,Cj,YZv),(Ny,Cj,YZw),(Ny,Cj,YZx),( Ny,Cj,YZy),(Ny,Cj,YZz),(Ny,Cj,YZaa),(Ny,Cj,YZab),(Ny,Cj,YZac),(Ny,Cj,YZad),(Ny,Cj,Y Zae),(Ny,Cj,YZaf),(Ny,Cj,YZag),(Nz,Ca,YZa),(Nz,Ca,YZb),(Nz,Ca,YZc),(Nz,Ca,YZd),(Nz, Ca,YZe),(Nz,Ca,YZf),(Nz,Ca,YZg),(Nz,Ca,YZh),(Nz,Ca,YZi),(Nz,Ca,YZj),(Nz,Ca,YZk),(N z,Ca,YZl),(Nz,Ca,YZm),(Nz,Ca,YZn),(Nz,Ca,YZo),(Nz,Ca,YZp),(Nz,Ca,YZq),(Nz,Ca,YZr), (Nz,Ca,YZs),(Nz,Ca,YZt),(Nz,Ca,YZu),(Nz,Ca,YZv),(Nz,Ca,YZw),(Nz,Ca,YZx),(Nz,Ca,YZ y),(Nz,Ca,YZz),(Nz,Ca,YZaa),(Nz,Ca,YZab),(Nz,Ca,YZac),(Nz,Ca,YZad),(Nz,Ca,YZae),(N z,Ca,YZaf),(Nz,Ca,YZag),(Nz,Cb,YZa),(Nz,Cb,YZb),(Nz,Cb,YZc),(Nz,Cb,YZd),(Nz,Cb,YZ e),(Nz,Cb,YZf),(Nz,Cb,YZg),(Nz,Cb,YZh),(Nz,Cb,YZi),(Nz,Cb,YZj),(Nz,Cb,YZk),(Nz,Cb, YZl),(Nz,Cb,YZm),(Nz,Cb,YZn),(Nz,Cb,YZo),(Nz,Cb,YZp),(Nz,Cb,YZq),(Nz,Cb,YZr),(Nz, Cb,YZs),(Nz,Cb,YZt),(Nz,Cb,YZu),(Nz,Cb,YZy),(Nz,Cb,YZw),(Nz,Cb,YZx),(Nz,Cb,YZy),( Nz,Cb,YZz),(Nz,Cb,YZaa),(Nz,Cb,YZab),(Nz,Cb,YZac),(Nz,Cb,YZad),(Nz,Cb,YZae),(Nz,C b,YZaf),(Nz,Cb,YZag),(Nz,Cc,YZa),(Nz,Cc,YZb),(Nz,Cc,YZc),(Nz,Cc,YZd),(Nz,Cc,YZe),( Nz,Cc,YZf),(Nz,Cc,YZg),(Nz,Cc,YZh),(Nz,Cc,YZi),(Nz,Cc,YZj),(Nz,Cc,YZk),(Nz,Cc,YZl), (Nz,Cc,YZm),(Nz,Cc,YZn),(Nz,Cc,YZo),(Nz,Cc,YZp),(Nz,Cc,YZq),(Nz,Cc,YZr),(Nz,Cc,Y Zs),(Nz,Cc,YZt),(Nz,Cc,YZu),(Nz,Cc,YZy),(Nz,Cc,YZw),(Nz,Cc,YZx),(Nz,Cc,YZy),(Nz,Cc ,YZz),(Nz,Cc,YZaa),(Nz,Cc,YZab),(Nz,Cc,YZac),(Nz,Cc,YZad),(Nz,Cc,YZae),(Nz,Cc,YZaf) ,(Nz,Cc,YZag),(Nz,Cd,YZa),(Nz,Cd,YZb),(Nz,Cd,YZc),(Nz,Cd,YZd),(Nz,Cd,YZe),(Nz,Cd, YZf),(Nz,Cd,YZg),(Nz,Cd,YZh),(Nz,Cd,YZi),(Nz,Cd,YZj),(Nz,Cd,YZic),(Nz,Cd,YZl),(Nz,C d,YZm),(Nz,Cd,YZn),(Nz,Cd,YZo),(Nz,Cd,YZp),(Nz,Cd,YZq),(Nz,Cd,YZr),(Nz,Cd,YZs),( Nz,Cd,YZt),(Nz,Cd,YZu),(Nz,Cd,YZy),(Nz,Cd,YZw),(Nz,Cd,YZx),(Nz,Cd,YZy),(Nz,Cd,Y Zz),(Nz,Cd,YZaa),(Nz,Cd,YZab),(Nz,Cd,YZac),(Nz,Cd,YZad),(Nz,Cd,YZae),(Nz,Cd,YZaf), (Nz,Cd,YZag),(Nz,Ce,YZa),(Nz,Ce,YZb),(Nz,Ce,YZc),(Nz,Ce,YZd),(Nz,Ce,YZe),(Nz,Ce,Y Zf),(Nz,Ce,YZg),(Nz,Ce,YZh),(Nz,Ce,YZi),(Nz,Ce,YZj),(Nz,Ce,YZk),(Nz,Ce,YZl),(Nz,Ce, YZm),(Nz,Ce,YZn),(Nz,Ce,YZo),(Nz,Ce,YZp),(Nz,Ce,YZq),(Nz,Ce,YZr),(Nz,Ce,YZs),(Nz, Ce,YZt),(Nz,Ce,YZu),(Nz,Ce,YZy),(Nz,Ce,YZw),(Nz,Ce,YZx),(Nz,Ce,YZy),(Nz,Ce,YZz),( Nz,Ce,YZaa),(Nz,Ce,YZab),(Nz,Ce,YZac),(Nz,Ce,YZad),(Nz,Ce,YZae),(Nz,Ce,YZaf),(Nz,C e,YZag),(Nz,Cf,YZa),(Nz,Cf,YZb),(Nz,Cf,YZc),(Nz,Cf,YZd),(Nz,Cf,YZe),(Nz,Cf,YZf),(Nz, Cf,YZg),(Nz,Cf,YZh),(Nz,Cf,YZi),(Nz,Cf,YZj),(Nz,Cf,YZk),(Nz,Cf,YZl),(Nz,Cf,YZm),(Nz, Cf,YZn),(Nz,Cf,YZo),(Nz,Cf,YZp),(Nz,Cf,YZq),(Nz,Cf,YZr),(Nz,Cf,YZs),(Nz,Cf,YZt),(Nz, Cf,YZu),(Nz,Cf,YZy),(Nz,Cf,YZw),(Nz,Cf,YZx),(Nz,Cf,YZy),(Nz,Cf,YZz),(Nz,Cf,YZaa),( Nz,Cf,YZab),(Nz,Cf,YZac),(Nz,Cf,YZad),(Nz,Cf,YZae),(Nz,Cf,YZaf),(Nz,Cf,YZag),(Nz,Cg ,YZa),(Nz,Cg,YZb),(Nz,Cg,YZc),(Nz,Cg,YZd),(Nz,Cg,YZe),(Nz,Cg,YZf),(Nz,Cg,YZg),(Nz, Cg,YZh),(Nz,Cg,YZi),(Nz,Cg,YZj),(Nz,Cg,YZk),(Nz,Cg,YZl),(Nz,Cg,YZm),(Nz,Cg,YZn),( Nz,Cg,YZo),(Nz,Cg,YZp),(Nz,Cg,YZq),(Nz,Cg,YZr),(Nz,Cg,YZs),(Nz,Cg,YZt),(Nz,Cg,YZ u),(Nz,Cg,YZv),(Nz,Cg,YZw),(Nz,Cg,YZx),(Nz,Cg,YZy),(Nz,Cg,YZz),(Nz,Cg,YZaa),(Nz,C g,YZab),(Nz,Cg,YZac),(Nz,Cg,YZad),(Nz,Cg,YZae),(Nz,Cg,YZaf),(Nz,Cg,YZag),(Nz,Ch,Y Za),(Nz,Ch,YZb),(Nz,Ch,YZc),(Nz,Ch,YZd),(Nz,Ch,YZe),(Nz,Ch,YZf),(Nz,Ch,YZg),(Nz,C h,YZh),(Nz,Ch,YZi),(Nz,Ch,YZj),(Nz,Ch,YZk),(Nz,Ch,YZl),(Nz,Ch,YZm),(Nz,Ch,YZn),(N z,Ch,YZo),(Nz,Ch,YZp),(Nz,Ch,YZq),(Nz,Ch,YZr),(Nz,Ch,YZs),(Nz,Ch,YZt),(Nz,Ch,YZu), (Nz,Ch,YZv),(Nz,Ch,YZw),(Nz,Ch,YZx),(Nz,Ch,YZy),(Nz,Ch,YZz),(Nz,Ch,YZaa),(Nz,Ch, Yzab),(Nz,Ch,YZac),(Nz,Ch,YZad),(Nz,Ch,YZae),(Nz,Ch,YZaf),(Nz,Ch,YZag),(Nz,Ci,YZa) ,(Nz,Ci,YZb),(Nz,Ci,YZc),(Nz,Ci,YZd),(Nz,Ci,YZe),(Nz,Ci,YZf),(Nz,Ci,YZg),(Nz,Ci,YZh), (Nz,Ci,YZi),(Nz,Ci,YZj),(Nz,Ci,YZk),(Nz,Ci,YZl),(Nz,Ci,YZm),(Nz,Ci,YZn),(Nz,Ci,YZo),( Nz,Ci,YZp),(Nz,Ci,YZq),(Nz,Ci,YZr),(Nz,Ci,YZs),(Nz,Ci,YZf),(Nz,Ci,YZu),(Nz,Ci,YZy),( Nz,Ci,YZw),(Nz,Ci,YZx),(Nz,Ci,YZy),(Nz,Ci,YZz),(Nz,Ci,YZaa),(Nz,Ci,YZab),(Nz,Ci,YZa c),(Nz,Ci,YZad),(Nz,Ci,YZae),(Nz,Ci,YZaf),(Nz,Ci,YZag),(Nz,Cj,YZa),(Nz,Cj,YZb),(Nz,Cj ,YZc),(Nz,Cj,YZd),(Nz,Cj,YZe),(Nz,Cj,YZf),(Nz,Cj,YZg),(Nz,Cj,YZh),(Nz,Cj,YZi),(Nz,Cj, YZj),(Nz,Cj,YZk),(Nz,Cj,YZl),(Nz,Cj,YZm),(Nz,Cj,YZn),(Nz,Cj,YZo),(Nz,Cj,YZp),(Nz,Cj, YZq),(Nz,Cj,YZr),(Nz,Cj,YZs),(Nz,Cj,YZt),(Nz,Cj,YZu),(Nz,Cj,YZy),(Nz,Cj,YZw),(Nz,Cj, YZx),(Nz,Cj,YZy),(Nz,Cj,YZz),(Nz,Cj,YZaa),(Nz,Cj,Yzab),(Nz,Cj,YZac),(Nz,Cj,YZad),(Nz ,Cj,YZae),(Nz,Cj,YZaf),(Nz,Cj,YZag),(Naa,Ca,YZa),(Naa,Ca,YZb),(Naa,Ca,YZc),(Naa,Ca, YZd),(Naa,Ca,YZe),(Naa,Ca,YZf),(Naa,Ca,YZg),(Naa,Ca,YZh),(Naa,Ca,YZi),(Naa,Ca,YZj), (Naa,Ca,YZk),(Naa,Ca,YZl),(Naa,Ca,YZm),(Naa,Ca,YZn),(Naa,Ca,YZo),(Naa,Ca,YZp),(Na a,Ca,YZq),(Naa,Ca,YZr),(Naa,Ca,YZs),(Naa,Ca,YZt),(Naa,Ca,YZu),(Naa,Ca,YZv),(Naa,Ca, YZw),(Naa,Ca,YZx),(Naa,Ca,YZy),(Naa,Ca,YZz),(Naa,Ca,YZaa),(Naa,Ca,YZab),(Naa,Ca,Y Zac),(Naa,Ca,YZad),(Naa,Ca,YZae),(Naa,Ca,YZaf),(Naa,Ca,YZag),(Naa,Cb,YZa),(Naa,Cb, YZb),(Naa,Cb,YZc),(Naa,Cb,YZd),(Naa,Cb,YZe),(Naa,Cb,YZf),(Naa,Cb,YZg),(Naa,Cb,YZ h),(Naa,Cb,YZi),(Naa,Cb,YZj),(Naa,Cb,YZk),(Naa,Cb,YZl),(Naa,Cb,YZm),(Naa,Cb,YZn),( Naa,Cb,YZo),(Naa,Cb,YZp),(Naa,Cb,YZq),(Naa,Cb,YZr),(Naa,Cb,YZs),(Naa,Cb,YZt),(Naa, Cb,YZu),(Naa,Cb,YZy),(Naa,Cb,YZw),(Naa,Cb,YZx),(Naa,Cb,YZy),(Naa,Cb,YZz),(Naa,Cb ,YZaa),(Naa,Cb,YZab),(Naa,Cb,YZac),(Naa,Cb,YZad),(Naa,Cb,YZae),(Naa,Cb,YZaf),(Naa, Cb,YZag),(Naa,Cc,YZa),(Naa,Cc,YZb),(Naa,Cc,YZc),(Naa,Cc,YZd),(Naa,Cc,YZe),(Naa,Cc, YZf),(Naa,Cc,YZg),(Naa,Cc,YZh),(Naa,Cc,YZi),(Naa,Cc,YZj),(Naa,Cc,YZk),(Naa,Cc,YZl), (Naa,Cc,YZm),(Naa,Cc,YZn),(Naa,Cc,YZo),(Naa,Cc,YZp),(Naa,Cc,YZq),(Naa,Cc,YZr),(Na a,Cc,YZs),(Naa,Cc,YZt),(Naa,Cc,YZu),(Naa,Cc,YZy),(Naa,Cc,YZw),(Naa,Cc,YZx),(Naa,Cc ,YZy),(Naa,Cc,YZz),(Naa,Cc,YZaa),(Naa,Cc,YZab),(Naa,Cc,YZac),(Naa,Cc,YZad),(Naa,Cc ,YZae),(Naa,Cc,YZaf),(Naa,Cc,YZag),(Naa,Cd,YZa),(Naa,Cd,YZb),(Naa,Cd,YZc),(Naa,Cd, YZd),(Naa,Cd,YZe),(Naa,Cd,YZf),(Naa,Cd,YZg),(Naa,Cd,YZh),(Naa,Cd,YZi),(Naa,Cd,YZj) ,(Naa,Cd,YZk),(Naa,Cd,YZl),(Naa,Cd,YZm),(Naa,Cd,YZn),(Naa,Cd,YZo),(Naa,Cd,YZp),( Naa,Cd,YZq),(Naa,Cd,YZr),(Naa,Cd,YZs),(Naa,Cd,Ya),(Naa,Cd,YZu),(Naa,Cd,YZy),(Naa, Cd,YZw),(Naa,Cd,YZx),(Naa,Cd,YZy),(Naa,Cd,YZz),(Naa,Cd,YZaa),(Naa,Cd,YZab),(Naa, Cd,YZac),(Naa,Cd,YZad),(Naa,Cd,YZae),(Naa,Cd,YZaf),(Naa,Cd,YZag),(Naa,Ce,YZa),(Na a,Ce,YZb),(Naa,Ce,YZc),(Naa,Ce,YZd),(Naa,Ce,YZe),(Naa,Ce,YZf),(Naa,Ce,YZg),(Naa,Ce, YZh),(Naa,Ce,YZi),(Naa,Ce,YZj),(Naa,Ce,YZk),(Naa,Ce,YZl),(Naa,Ce,YZm),(Naa,Ce,YZn) ,(Naa,Ce,YZo),(Naa,Ce,YZp),(Naa,Ce,YZq),(Naa,Ce,YZr),(Naa,Ce,YZs),(Naa,Ce,YZt),(Naa ,Ce,YZu),(Naa,Ce,YZy),(Naa,Ce,YZw),(Naa,Ce,YZx),(Naa,Ce,YZy),(Naa,Ce,YZz),(Naa,Ce, YZaa),(Naa,Ce,YZab),(Naa,Ce,YZac),(Naa,Ce,YZad),(Naa,Ce,YZae),(Naa,Ce,YZaf),(Naa,C e,YZag),(Naa,Cf,YZa),(Naa,Cf,YZb),(Naa,Cf,YZc),(Naa,Cf,YZcl),(Naa,Cf,YZe),(Naa,Cf,YZ f),(Naa,Cf,YZg),(Naa,Cf,YZh),(Naa,Cf,YZi),(Naa,Cf,YZj),(Naa,Cf,YZk),(Naa,Cf,YZl),(Naa, Cf,YZm),(Naa,Cf,YZn),(Naa,Cf,YZo),(Naa,Cf,YZp),(Naa,Cf,YZq),(Naa,Cf,YZr),(Naa,Cf,Y Zs),(Naa,Cf,YZt),(Naa,Cf,YZu),(Naa,Cf,YZy),(Naa,Cf,YZw),(Naa,Cf,YZx),(Naa,Cf,YZy),( Naa,Cf,YZz),(Naa,Cf,YZaa),(Naa,Cf,YZab),(Naa,Cf,YZac),(Naa,Cf,YZad),(Naa,Cf,YZae),( Naa,Cf,YZaf),(Naa,Cf,YZag),(Naa,Cg,YZa),(Naa,Cg,YZb),(Naa,Cg,YZc),(Naa,Cg,YZd),(Na a,Cg,YZe),(Naa,Cg,YZf),(Naa,Cg,YZg),(Naa,Cg,YZh),(Naa,Cg,YZi),(Naa,Cg,YZj),(Naa,Cg, YZk),(Naa,Cg,YZl),(Naa,Cg,YZm),(Naa,Cg,YZn),(Naa,Cg,YZo),(Naa,Cg,YZp),(Naa,Cg,YZ q),(Naa,Cg,YZr),(Naa,Cg,YZs),(Naa,Cg,YZt),(Naa,Cg,YZu),(Naa,Cg,YZA),(Naa,Cg,YZw),( Naa,Cg,YZx),(Naa,Cg,YZy),(Naa,Cg,YZz),(Naa,Cg,YZaa),(Naa,Cg,YZab),(Naa,Cg,YZac),( Naa,Cg,YZad),(Naa,Cg,YZae),(Naa,Cg,YZaf),(Naa,Cg,YZag),(Naa,Ch,YZa),(Naa,Ch,YZb), (Naa,Ch,YZc),(Naa,Ch,YZd),(Naa,Ch,YZe),(Naa,Ch,YZf),(Naa,Ch,YZg),(Naa,Ch,YZh),(Na a,Ch,YZi),(Naa,Ch,YZj),(Naa,Ch,YZk),(Naa,Ch,YZl),(Naa,Ch,YZm),(Naa,Ch,YZn),(Naa,C h,YZo),(Naa,Ch,YZp),(Naa,Ch,YZq),(Naa,Ch,YZr),(Naa,Ch,YZs),(Naa,Ch,YZt),(Naa,Ch,Y Zu),(Naa,Ch,YZNT),(Naa,Ch,YZw),(Naa,Ch,YZx),(Naa,Ch,YZy),(Naa,Ch,YZz),(Naa,Ch,YZa a),(Naa,Ch,YZab),(Naa,Ch,YZac),(Naa,Ch,YZad),(Naa,Ch,YZae),(Naa,Ch,YZaf),(Naa,Ch,Y Zag),(Naa,Ci,YZa),(Naa,Ci,YZb),(Naa,Ci,YZc),(Naa,Ci,YZd),(Naa,Ci,YZe),(Naa,Ci,YZf),( Naa,Ci,YZg),(Naa,Ci,YZh),(Naa,Ci,YZi),(Naa,Ci,YZj),(Naa,Ci,YZk),(Naa,Ci,YZl),(Naa,Ci, YZm),(Naa,Ci,YZn),(Naa,Ci,YZo),(Naa,Ci,YZp),(Naa,Ci,YZq),(Naa,Ci,YZr),(Naa,Ci,YZs),( Naa,Ci,YZf),(Naa,Ci,YZu),(Naa,Ci,YZy),(Naa,Ci,YZw),(Naa,Ci,YZx),(Naa,Ci,YZy),(Naa,C i,YZz),(Naa,Ci,YZaa),(Naa,Ci,YZab),(Naa,Ci,YZac),(Naa,Ci,YZad),(Naa,Ci,YZae),(Naa,Ci, YZaf),(Naa,Ci,YZag),(Naa,Cj,YZa),(Naa,Cj,YZb),(Naa,Cj,YZc),(Naa,Cj,YZd),(Naa,Cj,YZe) ,(Naa,Cj,YZf),(Naa,Cj,YZg),(Naa,Cj,YZh),(Naa,Cj,YZi),(Naa,Cj,YZj),(Naa,Cj,YZk),(Naa,Cj ,YZl),(Naa,Cj,YZm),(Naa,Cj,YZn),(Naa,Cj,YZo),(Naa,Cj,YZp),(Naa,Cj,YZq),(Naa,Cj,YZr), (Naa,Cj,YZs),(Naa,Cj,YZt),(Naa,Cj,YZu),(Naa,Cj,YZy),(Naa,Cj,YZw),(Naa,Cj,YZx),(Naa,C j,YZy),(Naa,Cj,YZz),(Naa,Cj,YZaa),(Naa,Cj,YZab),(Naa,Cj,YZac),(Naa,Cj,YZad),(Naa,Cj, YZae),(Naa,Cj,YZaf),(Naa,Cj,YZag),(Nab,Ca,YZa),(Nab,Ca,YZb),(Nab,Ca,YZc),(Nab,Ca,Y Zd),(Nab,Ca,YZe),(Nab,Ca,YZf),(Nab,Ca,YZg),(Nab,Ca,YZh),(Nab,Ca,YZi),(Nab,Ca,YZj),( Nab,Ca,Ya),(Nab,Ca,YZl),(Nab,Ca,YZm),(Nab,Ca,YZn),(Nab,Ca,YZo),(Nab,Ca,YZp),(Na b,Ca,YZq),(Nab,Ca,YZr),(Nab,Ca,YZs),(Nab,Ca,Ya),(Nab,Ca,YZu),(Nab,Ca,YZy),(Nab,Ca ,YZw),(Nab,Ca,YZx),(Nab,Ca,YZy),(Nab,Ca,YZz),(Nab,Ca,YZaa),(Nab,Ca,YZab),(Nab,Ca, YZac),(Nab,Ca,YZad),(Nab,Ca,YZae),(Nab,Ca,YZaf),(Nab,Ca,YZag),(Nab,Cb,YZa),(Nab,C b,YZb),(Nab,Cb,YZc),(Nab,Cb,Yal),(Nab,Cb,YZe),(Nab,Cb,YZf),(Nab,Cb,YZg),(Nab,Cb, YZh),(Nab,Cb,YZi),(Nab,Cb,YZj),(Nab,Cb,YZk),(Nab,Cb,YZl),(Nab,Cb,YZm),(Nab,Cb,YZ n),(Nab,Cb,YZo),(Nab,Cb,YZp),(Nab,Cb,YZq),(Nab,Cb,YZr),(Nab,Cb,YZs),(Nab,Cb,YZt),( Nab,Cb,YZu),(Nab,Cb,YZy),(Nab,Cb,YZw),(Nab,Cb,YZx),(Nab,Cb,YZy),(Nab,Cb,YZz),(N ab,Cb,YZaa),(Nab,Cb,YZab),(Nab,Cb,YZac),(Nab,Cb,YZad),(Nab,Cb,YZae),(Nab,Cb,YZaf) ,(Nab,Cb,YZag),(Nab,Cc,YZa),(Nab,Cc,YZb),(Nab,Cc,YZc),(Nab,Cc,YZd),(Nab,Cc,YZe),( Nab,Cc,YZf),(Nab,Cc,YZg),(Nab,Cc,YZh),(Nab,Cc,YZi),(Nab,Cc,YZj),(Nab,Cc,YZk),(Nab, Cc,YZl),(Nab,Cc,YZm),(Nab,Cc,YZn),(Nab,Cc,YZo),(Nab,Cc,YZp),(Nab,Cc,YZq),(Nab,Cc, YZr),(Nab,Cc,YZs),(Nab,Cc,YZt),(Nab,Cc,YZu),(Nab,Cc,YZy),(Nab,Cc,YZw),(Nab,Cc,YZ x),(Nab,Cc,YZy),(Nab,Cc,YZz),(Nab,Cc,YZaa),(Nab,Cc,YZab),(Nab,Cc,YZac),(Nab,Cc,YZ ad),(Nab,Cc,YZae),(Nab,Cc,YZaf),(Nab,Cc,YZag),(Nab,Cd,YZa),(Nab,Cd,YZb),(Nab,Cd,Y Zc),(Nab,Cd,YZd),(Nab,Cd,YZe),(Nab,Cd,YZf),(Nab,Cd,YZg),(Nab,Cd,YZh),(Nab,Cd,YZi) ,(Nab,Cd,YZj),(Nab,Cd,YZk),(Nab,Cd,YZl),(Nab,Cd,YZm),(Nab,Cd,YZn),(Nab,Cd,YZo),( Nab,Cd,YZp),(Nab,Cd,YZq),(Nab,Cd,YZr),(Nab,Cd,YZs),(Nab,Cd,YZt),(Nab,Cd,YZu),(Na b,Cd,YZy),(Nab,Cd,YZw),(Nab,Cd,YZx),(Nab,Cd,YZy),(Nab,Cd,YZz),(Nab,Cd,YZaa),(Nab ,Cd,YZab),(Nab,Cd,YZac),(Nab,Cd,YZad),(Nab,Cd,YZae),(Nab,Cd,YZaf),(Nab,Cd,YZag),( Nab,Ce,YZa),(Nab,Ce,YZb),(Nab,Ce,YZc),(Nab,Ce,YZd),(Nab,Ce,YZe),(Nab,Ce,Ya),(Nab ,Ce,YZg),(Nab,Ce,YZh),(Nab,Ce,YZi),(Nab,Ce,YZj),(Nab,Ce,YZk),(Nab,Ce,YZl),(Nab,Ce, YZm),(Nab,Ce,YZn),(Nab,Ce,YZo),(Nab,Ce,YZp),(Nab,Ce,YZq),(Nab,Ce,YZr),(Nab,Ce,YZ s),(Nab,Ce,YZf),(Nab,Ce,YZu),(Nab,Ce,YZy),(Nab,Ce,YZw),(Nab,Ce,YZx),(Nab,Ce,YZy),( Nab,Ce,YZz),(Nab,Ce,YZaa),(Nab,Ce,YZab),(Nab,Ce,YZac),(Nab,Ce,YZad),(Nab,Ce,YZae) ,(Nab,Ce,YZaf),(Nab,Ce,YZag),(Nab,Cf,YZa),(Nab,Cf,YZb),(Nab,Cf,YZc),(Nab,Cf,YZd),( Nab,Cf,YZe),(Nab,Cf,YZf),(Nab,Cf,YZg),(Nab,Cf,YZh),(Nab,Cf,YZi),(Nab,Cf,YZj),(Nab,C f,YZk),(Nab,Cf,YZl),(Nab,Cf,YZm),(Nab,Cf,YZn),(Nab,Cf,YZo),(Nab,Cf,YZp),(Nab,Cf,YZ q),(Nab,Cf,YZr),(Nab,Cf,YZs),(Nab,Cf,YZt),(Nab,Cf,YZu),(Nab,Cf,YZy),(Nab,Cf,YZw),(N ab,Cf,YZx),(Nab,Cf,YZy),(Nab,Cf,YZz),(Nab,Cf,YZaa),(Nab,Cf,YZab),(Nab,Cf,YZac),(Nab ,Cf,YZad),(Nab,Cf,YZae),(Nab,Cf,YZaf),(Nab,Cf,YZag),(Nab,Cg,YZa),(Nab,Cg,YZb),(Nab, Cg,YZc),(Nab,Cg,YZd),(Nab,Cg,YZe),(Nab,Cg,YZf),(Nab,Cg,YZg),(Nab,Cg,YZh),(Nab,Cg ,YZi),(Nab,Cg,YZj),(Nab,Cg,YZk),(Nab,Cg,YZl),(Nab,Cg,YZm),(Nab,Cg,YZn),(Nab,Cg,Y Zo),(Nab,Cg,YZp),(Nab,Cg,YZq),(Nab,Cg,YZr),(Nab,Cg,YZs),(Nab,Cg,YZt),(Nab,Cg,YZu) ,(Nab,Cg,YZy),(Nab,Cg,YZw),(Nab,Cg,YZx),(Nab,Cg,YZy),(Nab,Cg,YZz),(Nab,Cg,YZaa), (Nab,Cg,YZab),(Nab,Cg,YZac),(Nab,Cg,YZad),(Nab,Cg,YZae),(Nab,Cg,YZaf),(Nab,Cg,YZ ag),(Nab,Ch,YZa),(Nab,Ch,YZb),(Nab,Ch,YZc),(Nab,Ch,YZd),(Nab,Ch,YZe),(Nab,Ch,YZf) ,(Nab,Ch,YZg),(Nab,Ch,YZh),(Nab,Ch,YZi),(Nab,Ch,YZj),(Nab,Ch,YZk),(Nab,Ch,YZl),(Na b,Ch,YZm),(Nab,Ch,YZn),(Nab,Ch,YZo),(Nab,Ch,YZp),(Nab,Ch,YZq),(Nab,Ch,YZr),(Nab, Ch,YZs),(Nab,Ch,YZt),(Nab,Ch,YZu),(Nab,Ch,YZv),(Nab,Ch,YZw),(Nab,Ch,YZx),(Nab,Ch ,YZy),(Nab,Ch,YZz),(Nab,Ch,YZaa),(Nab,Ch,YZab),(Nab,Ch,YZac),(Nab,Ch,YZad),(Nab,C h,YZae),(Nab,Ch,YZaf),(Nab,Ch,YZag),(Nab,Ci,YZa),(Nab,Ci,YZb),(Nab,Ci,YZc),(Nab,Ci, YZd),(Nab,Ci,YZe),(Nab,Ci,YZf),(Nab,Ci,YZg),(Nab,Ci,YZh),(Nab,Ci,YZi),(Nab,Ci,YZj),( Nab,Ci,YZk),(Nab,Ci,YZl),(Nab,Ci,YZm),(Nab,Ci,YZn),(Nab,Ci,YZo),(Nab,Ci,YZp),(Nab, Ci,YZq),(Nab,Ci,YZr),(Nab,Ci,YZs),(Nab,Ci,YZt),(Nab,Ci,YZu),(Nab,Ci,YZv),(Nab,Ci,YZ w),(Nab,Ci,YZx),(Nab,Ci,YZy),(Nab,Ci,YZz),(Nab,Ci,YZaa),(Nab,Ci,YZab),(Nab,Ci,YZac) ,(Nab,Ci,YZad),(Nab,Ci,YZae),(Nab,Ci,YZaf),(Nab,Ci,YZag),(Nab,Cj,YZa),(Nab,Cj,YZb),( Nab,Cj,YZc),(Nab,Cj,YZd),(Nab,Cj,YZe),(Nab,Cj,YZf),(Nab,Cj,YZg),(Nab,Cj,YZh),(Nab,C j,YZi),(Nab,Cj,YZj),(Nab,Cj,YZk),(Nab,Cj,YZl),(Nab,Cj,YZm),(Nab,Cj,YZn),(Nab,Cj,YZo) ,(Nab,Cj,YZp),(Nab,Cj,YZq),(Nab,Cj,YZr),(Nab,Cj,YZs),(Nab,Cj,YZt),(Nab,Cj,YZu),(Nab, Cj,YZv),(Nab,Cj,YZw),(Nab,Cj,YZx),(Nab,Cj,YZy),(Nab,Cj,YZz),(Nab,Cj,YZaa),(Nab,Cj, YZab),(Nab,Cj,YZac),(Nab,Cj,YZad),(Nab,Cj,YZae),(Nab,Cj,YZaf),(Nab,Cj,YZag),(Nac,Ca ,YZa),(Nac,Ca,YZb),(Nac,Ca,YZc),(Nac,Ca,YZd),(Nac,Ca,YZe),(Nac,Ca,YZf),(Nac,Ca,YZg) ,(Nac,Ca,YZh),(Nac,Ca,YZi),(Nac,Ca,YZj),(Nac,Ca,YZk),(Nac,Ca,YZl),(Nac,Ca,YZm),(Na c,Ca,YZn),(Nac,Ca,YZo),(Nac,Ca,YZp),(Nac,Ca,YZq),(Nac,Ca,YZr),(Nac,Ca,YZs),(Nac,Ca, YZt),(Nac,Ca,YZu),(Nac,Ca,YZv),(Nac,Ca,YZw),(Nac,Ca,YZx),(Nac,Ca,YZy),(Nac,Ca,YZz) ,(Nac,Ca,YZaa),(Nac,Ca,YZab),(Nac,Ca,YZac),(Nac,Ca,YZad),(Nac,Ca,YZae),(Nac,Ca,YZ af),(Nac,Ca,YZag),(Nac,Cb,YZa),(Nac,Cb,YZb),(Nac,Cb,YZc),(Nac,Cb,YZd),(Nac,Cb,YZe) ,(Nac,Cb,YZf),(Nac,Cb,YZg),(Nac,Cb,YZh),(Nac,Cb,YZi),(Nac,Cb,YZj),(Nac,Cb,YZk),(Na c,Cb,YZl),(Nac,Cb,YZm),(Nac,Cb,YZn),(Nac,Cb,YZo),(Nac,Cb,YZp),(Nac,Cb,YZq),(Nac,C b,YZr),(Nac,Cb,YZs),(Nac,Cb,YZt),(Nac,Cb,YZu),(Nac,Cb,YZv),(Nac,Cb,YZw),(Nac,Cb,Y Zx),(Nac,Cb,YZy),(Nac,Cb,YZz),(Nac,Cb,YZaa),(Nac,Cb,YZab),(Nac,Cb,YZac),(Nac,Cb,Y Zad),(Nac,Cb,YZae),(Nac,Cb,YZaf),(Nac,Cb,YZag),(Nac,Cc,YZa),(Nac,Cc,YZb),(Nac,Cc,Y Zc),(Nac,Cc,YZd),(Nac,Cc,YZe),(Nac,Cc,Ya),(Nac,Cc,YZg),(Nac,Cc,YZh),(Nac,Cc,YZi),( Nac,Cc,YZj),(Nac,Cc,YZk),(Nac,Cc,YZl),(Nac,Cc,YZm),(Nac,Cc,YZn),(Nac,Cc,YZo),(Nac, Cc,YZp),(Nac,Cc,YZq),(Nac,Cc,YZr),(Nac,Cc,YZs),(Nac,Cc,YZt),(Nac,Cc,YZu),(Nac,Cc,Y Zv),(Nac,Cc,YZw),(Nac,Cc,YZx),(Nac,Cc,YZy),(Nac,Cc,YZz),(Nac,Cc,YZaa),(Nac,Cc,YZa b),(Nac,Cc,YZac),(Nac,Cc,YZad),(Nac,Cc,YZae),(Nac,Cc,YZaf),(Nac,Cc,YZag),(Nac,Cd,Y Za),(Nac,Cd,YZb),(Nac,Cd,YZc),(Nac,Cd,YZd),(Nac,Cd,YZe),(Nac,Cd,YZf),(Nac,Cd,YZg), (Nac,Cd,Ya),(Nac,Cd,YZi),(Nac,Cd,YZj),(Nac,Cd,YZk),(Nac,Cd,YZl),(Nac,Cd,YZm),(Na c,Cd,YZn),(Nac,Cd,YZo),(Nac,Cd,YZp),(Nac,Cd,YZq),(Nac,Cd,YZr),(Nac,Cd,YZs),(Nac,C d,YZf),(Nac,Cd,YZu),(Nac,Cd,YZv),(Nac,Cd,YZw),(Nac,Cd,YZx),(Nac,Cd,YZy),(Nac,Cd, YZz),(Nac,Cd,YZaa),(Nac,Cd,YZab),(Nac,Cd,YZac),(Nac,Cd,YZad),(Nac,Cd,YZae),(Nac,C d,YZaf),(Nac,Cd,YZag),(Nac,Ce,YZa),(Nac,Ce,YZb),(Nac,Ce,YZc),(Nac,Ce,YZd),(Nac,Ce, YZe),(Nac,Ce,YZl),(Nac,Ce,YZg),(Nac,Ce,YZh),(Nac,Ce,YZi),(Nac,Ce,YZj),(Nac,Ce,YZk), (Nac,Ce,YZl),(Nac,Ce,YZm),(Nac,Ce,YZn),(Nac,Ce,YZo),(Nac,Ce,YZp),(Nac,Ce,YZq),(Na c,Ce,YZr),(Nac,Ce,YZs),(Nac,Ce,YZt),(Nac,Ce,YZu),(Nac,Ce,YZy),(Nac,Ce,YZw),(Nac,Ce, YZx),(Nac,Ce,YZy),(Nac,Ce,YZz),(Nac,Ce,YZaa),(Nac,Ce,YZab),(Nac,Ce,YZac),(Nac,Ce, YZad),(Nac,Ce,YZae),(Nac,Ce,YZaf),(Nac,Ce,YZag),(Nac,Cf,YZa),(Nac,Cf,YZb),(Nac,Cf, YZc),(Nac,Cf,YZd),(Nac,Cf,YZe),(Nac,Cf,YZf),(Nac,Cf,YZg),(Nac,Cf,YZh),(Nac,Cf,YZi),( Nac,Cf,YZj),(Nac,Cf,YZk),(Nac,Cf,YZl),(Nac,Cf,YZm),(Nac,Cf,YZn),(Nac,Cf,YZo),(Nac,C f,YZp),(Nac,Cf,YZq),(Nac,Cf,YZr),(Nac,Cf,YZs),(Nac,Cf,YZt),(Nac,Cf,YZu),(Nac,Cf,YZy) ,(Nac,Cf,YZw),(Nac,Cf,YZx),(Nac,Cf,YZy),(Nac,Cf,YZz),(Nac,Cf,YZaa),(Nac,Cf,YZab),(N ac,Cf,YZac),(Nac,Cf,YZad),(Nac,Cf,YZae),(Nac,Cf,YZaf),(Nac,Cf,YZag),(Nac,Cg,YZa),(N ac,Cg,YZb),(Nac,Cg,YZc),(Nac,Cg,YZd),(Nac,Cg,YZe),(Nac,Cg,YZf),(Nac,Cg,YZg),(Nac, Cg,YZh),(Nac,Cg,YZi),(Nac,Cg,YZj),(Nac,Cg,YZk),(Nac,Cg,YZl),(Nac,Cg,YZm),(Nac,Cg, YZn),(Nac,Cg,YZo),(Nac,Cg,YZp),(Nac,Cg,YZq),(Nac,Cg,YZr),(Nac,Cg,YZs),(Nac,Cg,YZt) ,(Nac,Cg,YZu),(Nac,Cg,YZA/),(Nac,Cg,YZw),(Nac,Cg,YZx),(Nac,Cg,YZy),(Nac,Cg,YZz),( Nac,Cg,YZaa),(Nac,Cg,YZab),(Nac,Cg,YZac),(Nac,Cg,YZad),(Nac,Cg,YZae),(Nac,Cg,YZaf) ,(Nac,Cg,YZag),(Nac,Ch,YZa),(Nac,Ch,YZb),(Nac,Ch,YZc),(Nac,Ch,YZd),(Nac,Ch,YZe),( Nac,Ch,YZf),(Nac,Ch,YZg),(Nac,Ch,YZh),(Nac,Ch,YZi),(Nac,Ch,YZj),(Nac,Ch,YZk),(Nac, Ch,YZl),(Nac,Ch,YZm),(Nac,Ch,YZn),(Nac,Ch,YZo),(Nac,Ch,YZp),(Nac,Ch,YZq),(Nac,Ch ,YZr),(Nac,Ch,YZs),(Nac,Ch,YZt),(Nac,Ch,YZu),(Nac,Ch,YZy),(Nac,Ch,YZw),(Nac,Ch,YZ x),(Nac,Ch,YZy),(Nac,Ch,YZz),(Nac,Ch,YZaa),(Nac,Ch,YZab),(Nac,Ch,YZac),(Nac,Ch,YZ ad),(Nac,Ch,YZae),(Nac,Ch,YZaf),(Nac,Ch,YZag),(Nac,Ci,YZa),(Nac,Ci,YZb),(Nac,Ci,YZc) ,(Nac,Ci,YZd),(Nac,Ci,YZe),(Nac,Ci,YZf),(Nac,Ci,YZg),(Nac,Ci,YZh),(Nac,Ci,YZi),(Nac, Ci,YZj),(Nac,Ci,YZk),(Nac,Ci,YZl),(Nac,Ci,YZm),(Nac,Ci,YZn),(Nac,Ci,YZo),(Nac,Ci,YZ p),(Nac,Ci,YZq),(Nac,Ci,YZr),(Nac,Ci,YZs),(Nac,Ci,YZt),(Nac,Ci,YZu),(Nac,Ci,YZv),(Nac, Ci,YZw),(Nac,Ci,YZx),(Nac,Ci,YZy),(Nac,Ci,YZz),(Nac,Ci,YZaa),(Nac,Ci,YZab),(Nac,Ci, YZac),(Nac,Ci,YZad),(Nac,Ci,YZae),(Nac,Ci,YZaf),(Nac,Ci,YZag),(Nac,Cj,YZa),(Nac,Cj,Y Zb),(Nac,Cj,YZc),(Nac,Cj,YZd),(Nac,Cj,YZe),(Nac,Cj,YZf),(Nac,Cj,YZg),(Nac,Cj,YZh),(N ac,Cj,YZi),(Nac,Cj,YZj),(Nac,Cj,YZk),(Nac,Cj,YZl),(Nac,Cj,YZm),(Nac,Cj,YZn),(Nac,Cj,Y Zo),(Nac,Cj,YZp),(Nac,Cj,YZq),(Nac,Cj,YZr),(Nac,Cj,YZs),(Nac,Cj,YZt),(Nac,Cj,YZu),(Na c,Cj,YZy),(Nac,Cj,YZw),(Nac,Cj,YZx),(Nac,Cj,YZy),(Nac,Cj,YZz),(Nac,Cj,YZaa),(Nac,Cj, YZab),(Nac,Cj,YZac),(Nac,Cj,YZad),(Nac,Cj,YZae),(Nac,Cj,YZaf),(Nac,Cj,YZag),(Nad,Ca, YZa),(Nad,Ca,YZb),(Nad,Ca,YZc),(Nad,Ca,YZd),(Nad,Ca,YZe),(Nad,Ca,YZf),(Nad,Ca,YZ g),(Nad,Ca,YZh),(Nad,Ca,YZi),(Nad,Ca,YZj),(Nad,Ca,YZk),(Nad,Ca,YZl),(Nad,Ca,YZm),( Nad,Ca,YZn),(Nad,Ca,YZo),(Nad,Ca,YZp),(Nad,Ca,YZq),(Nad,Ca,YZr),(Nad,Ca,YZs),(Nad ,Ca,YZt),(Nad,Ca,YZu),(Nad,Ca,YZy),(Nad,Ca,YZw),(Nad,Ca,YZx),(Nad,Ca,YZy),(Nad,Ca ,YZz),(Nad,Ca,YZaa),(Nad,Ca,YZab),(Nad,Ca,YZac),(Nad,Ca,YZad),(Nad,Ca,YZae),(Nad, Ca,YZaf),(Nad,Ca,YZag),(Nad,Cb,YZa),(Nad,Cb,YZb),(Nad,Cb,YZc),(Nad,Cb,YZd),(Nad, Cb,YZe),(Nad,Cb,YZf),(Nad,Cb,YZg),(Nad,Cb,YZh),(Nad,Cb,YZi),(Nad,Cb,YZj),(Nad,Cb, YZk),(Nad,Cb,YZl),(Nad,Cb,YZm),(Nad,Cb,YZn),(Nad,Cb,YZo),(Nad,Cb,YZp),(Nad,Cb,Y Zq),(Nad,Cb,YZr),(Nad,Cb,YZs),(Nad,Cb,YZt),(Nad,Cb,YZu),(Nad,Cb,YZy),(Nad,Cb,YZw) ,(Nad,Cb,YZx),(Nad,Cb,YZy),(Nad,Cb,YZz),(Nad,Cb,YZaa),(Nad,Cb,YZab),(Nad,Cb,YZa c),(Nad,Cb,YZad),(Nad,Cb,YZae),(Nad,Cb,YZaf),(Nad,Cb,YZag),(Nad,Cc,YZa),(Nad,Cc,Y Zb),(Nad,Cc,YZc),(Nad,Cc,YZd),(Nad,Cc,YZe),(Nad,Cc,Ya),(Nad,Cc,YZg),(Nad,Cc,YZh), (Nad,Cc,YZi),(Nad,Cc,YZj),(Nad,Cc,YZk),(Nad,Cc,YZl),(Nad,Cc,YZm),(Nad,Cc,YZn),(Na d,Cc,YZo),(Nad,Cc,YZp),(Nad,Cc,YZq),(Nad,Cc,YZr),(Nad,Cc,YZs),(Nad,Cc,YZt),(Nad,Cc ,YZu),(Nad,Cc,YZv),(Nad,Cc,YZw),(Nad,Cc,YZx),(Nad,Cc,YZy),(Nad,Cc,YZz),(Nad,Cc,Y Zaa),(Nad,Cc,YZab),(Nad,Cc,YZac),(Nad,Cc,YZad),(Nad,Cc,YZae),(Nad,Cc,YZaf),(Nad,Cc ,YZag),(Nad,Cd,YZa),(Nad,Cd,YZb),(Nad,Cd,YZc),(Nad,Cd,YZd),(Nad,Cd,YZe),(Nad,Cd, Ya),(Nad,Cd,YZg),(Nad,Cd,YZh),(Nad,Cd,YZi),(Nad,Cd,YZj),(Nad,Cd,YZk),(Nad,Cd,YZl) ,(Nad,Cd,YZm),(Nad,Cd,YZn),(Nad,Cd,YZo),(Nad,Cd,YZp),(Nad,Cd,YZq),(Nad,Cd,YZr),( Nad,Cd,YZs),(Nad,Cd,YZt),(Nad,Cd,YZu),(Nad,Cd,YZv),(Nad,Cd,YZw),(Nad,Cd,YZx),(Na d,Cd,YZy),(Nad,Cd,YZz),(Nad,Cd,YZaa),(Nad,Cd,YZab),(Nad,Cd,YZac),(Nad,Cd,YZad),( Nad,Cd,YZae),(Nad,Cd,YZaf),(Nad,Cd,YZag),(Nad,Ce,YZa),(Nad,Ce,YZb),(Nad,Ce,YZc),( Nad,Ce,YZd),(Nad,Ce,YZe),(Nad,Ce,YZf),(Nad,Ce,YZg),(Nad,Ce,YZh),(Nad,Ce,YZi),(Nad ,Ce,YZj),(Nad,Ce,YZk),(Nad,Ce,YZl),(Nad,Ce,YZm),(Nad,Ce,YZn),(Nad,Ce,YZo),(Nad,Ce, YZp),(Nad,Ce,YZq),(Nad,Ce,YZr),(Nad,Ce,YZs),(Nad,Ce,YZf),(Nad,Ce,YZu),(Nad,Ce,YZy) ,(Nad,Ce,YZw),(Nad,Ce,YZx),(Nad,Ce,YZy),(Nad,Ce,YZz),(Nad,Ce,YZaa),(Nad,Ce,YZab) ,(Nad,Ce,YZac),(Nad,Ce,YZad),(Nad,Ce,YZae),(Nad,Ce,YZaf),(Nad,Ce,YZag),(Nad,Cf,YZa) ,(Nad,Cf,YZb),(Nad,Cf,YZc),(Nad,Cf,YZd),(Nad,Cf,YZe),(Nad,Cf,YZf),(Nad,Cf,YZg),(Na d,Cf,YZh),(Nad,Cf,YZi),(Nad,Cf,YZj),(Nad,Cf,YZk),(Nad,Cf,YZl),(Nad,Cf,YZm),(Nad,Cf, YZn),(Nad,Cf,YZo),(Nad,Cf,YZp),(Nad,Cf,YZq),(Nad,Cf,YZr),(Nad,Cf,YZs),(Nad,Cf,YZt), (Nad,Cf,YZu),(Nad,Cf,YZy),(Nad,Cf,YZw),(Nad,Cf,YZx),(Nad,Cf,YZy),(Nad,Cf,YZz),(Na d,Cf,YZaa),(Nad,Cf,YZab),(Nad,Cf,YZac),(Nad,Cf,YZad),(Nad,Cf,YZae),(Nad,Cf,YZaf),(N ad,Cf,YZag),(Nad,Cg,YZa),(Nad,Cg,YZb),(Nad,Cg,YZc),(Nad,Cg,YZd),(Nad,Cg,YZe),(Nad ,Cg,YZf),(Nad,Cg,YZg),(Nad,Cg,YZh),(Nad,Cg,YZi),(Nad,Cg,YZj),(Nad,Cg,YZk),(Nad,Cg, YZl),(Nad,Cg,YZm),(Nad,Cg,YZn),(Nad,Cg,YZo),(Nad,Cg,YZp),(Nad,Cg,YZq),(Nad,Cg,Y Zr),(Nad,Cg,YZs),(Nad,Cg,YZt),(Nad,Cg,YZu),(Nad,Cg,YZy),(Nad,Cg,YZw),(Nad,Cg,YZx ,(Nad,Cg,YZy),(Nad,Cg,YZz),(Nad,Cg,YZaa),(Nad,Cg,YZab),(Nad,Cg,YZac),(Nad,Cg,YZa d),(Nad,Cg,YZae),(Nad,Cg,YZaf),(Nad,Cg,YZag),(Nad,Ch,YZa),(Nad,Ch,YZb),(Nad,Ch,YZ c),(Nad,Ch,YZd),(Nad,Ch,YZe),(Nad,Ch,YZf),(Nad,Ch,YZg),(Nad,Ch,YZh),(Nad,Ch,YZi),( Nad,Ch,YZj),(Nad,Ch,YZk),(Nad,Ch,YZl),(Nad,Ch,YZm),(Nad,Ch,YZn),(Nad,Ch,YZo),(Na d,Ch,YZp),(Nad,Ch,YZq),(Nad,Ch,YZr),(Nad,Ch,YZs),(Nad,Ch,YZt),(Nad,Ch,YZu),(Nad,C h,YZy),(Nad,Ch,YZw),(Nad,Ch,YZx),(Nad,Ch,YZy),(Nad,Ch,YZz),(Nad,Ch,YZaa),(Nad,Ch ,YZab),(Nad,Ch,YZac),(Nad,Ch,YZad),(Nad,Ch,YZae),(Nad,Ch,YZaf),(Nad,Ch,YZag),(Nad ,Ci,YZa),(Nad,Ci,YZb),(Nad,Ci,YZc),(Nad,Ci,YZd),(Nad,Ci,YZe),(Nad,Ci,YZf),(Nad,Ci,YZ g),(Nad,Ci,YZh),(Nad,Ci,YZi),(Nad,Ci,YZj),(Nad,Ci,YZk),(Nad,Ci,YZl),(Nad,Ci,YZm),(Na d,Ci,YZn),(Nad,Ci,YZo),(Nad,Ci,YZp),(Nad,Ci,YZq),(Nad,Ci,YZr),(Nad,Ci,YZs),(Nad,Ci,Y Zt),(Nad,Ci,YZu),(Nad,Ci,YZy),(Nad,Ci,YZw),(Nad,Ci,YZx),(Nad,Ci,YZy),(Nad,Ci,YZz),( Nad,Ci,YZaa),(Nad,Ci,YZab),(Nad,Ci,YZac),(Nad,Ci,YZad),(Nad,Ci,YZae),(Nad,Ci,YZaf),( Nad,Ci,YZag),(Nad,Cj,YZa),(Nad,Cj,YZb),(Nad,Cj,YZc),(Nad,Cj,YZd),(Nad,Cj,YZe),(Nad, Cj,YZf),(Nad,Cj,YZg),(Nad,Cj,YZh),(Nad,Cj,YZi),(Nad,Cj,YZj),(Nad,Cj,YZk),(Nad,Cj,YZl) ,(Nad,Cj,YZm),(Nad,Cj,YZn),(Nad,Cj,YZo),(Nad,Cj,YZp),(Nad,Cj,YZq),(Nad,Cj,YZr),(Na d,Cj,YZs),(Nad,Cj,YZt),(Nad,Cj,YZu),(Nad,Cj,YZy),(Nad,Cj,YZw),(Nad,Cj,YZx),(Nad,Cj, YZy),(Nad,Cj,YZz),(Nad,Cj,YZaa),(Nad,Cj,YZab),(Nad,Cj,YZac),(Nad,Cj,YZad),(Nad,Cj,Y Zae),(Nad,Cj,YZaf),(Nad,Cj,YZag),(Nae,Ca,YZa),(Nae,Ca,YZb),(Nae,Ca,YZc),(Nae,Ca,YZ d),(Nae,Ca,YZe),(Nae,Ca,YZf),(Nae,Ca,YZg),(Nae,Ca,YEa),(Nae,Ca,YZi),(Nae,Ca,YZj),(N ae,Ca,YZk),(Nae,Ca,YZl),(Nae,Ca,YZm),(Nae,Ca,YZn),(Nae,Ca,YZo),(Nae,Ca,YZp),(Nae, Ca,YZq),(Nae,Ca,YZr),(Nae,Ca,YZs),(Nae,Ca,YZt),(Nae,Ca,YZu),(Nae,Ca,YZy),(Nae,Ca,Y Zw),(Nae,Ca,YZx),(Nae,Ca,YZy),(Nae,Ca,YZz),(Nae,Ca,YZaa),(Nae,Ca,YZab),(Nae,Ca,YZ ac),(Nae,Ca,YZad),(Nae,Ca,YZae),(Nae,Ca,YZaf),(Nae,Ca,YZag),(Nae,Cb,YZa),(Nae,Cb,Y Zb),(Nae,Cb,YZc),(Nae,Cb,YZd),(Nae,Cb,YZe),(Nae,Cb,YZf),(Nae,Cb,YZg),(Nae,Cb,YZh), (Nae,Cb,YZi),(Nae,Cb,YZj),(Nae,Cb,YZk),(Nae,Cb,YZl),(Nae,Cb,YZm),(Nae,Cb,YZn),(Na e,Cb,YZo),(Nae,Cb,YZp),(Nae,Cb,YZq),(Nae,Cb,YZr),(Nae,Cb,YZs),(Nae,Cb,YZt),(Nae,Cb ,YZu),(Nae,Cb,YZy),(Nae,Cb,YZw),(Nae,Cb,YZx),(Nae,Cb,YZy),(Nae,Cb,YZz),(Nae,Cb,Y Zaa),(Nae,Cb,YZab),(Nae,Cb,YZac),(Nae,Cb,YZad),(Nae,Cb,YZae),(Nae,Cb,YZaf),(Nae,Cb ,YZag),(Nae,Cc,YZa),(Nae,Cc,YZb),(Nae,Cc,YZc),(Nae,Cc,YZd),(Nae,Cc,YZe),(Nae,Cc,Y Zf),(Nae,Cc,YZg),(Nae,Cc,YZh),(Nae,Cc,YZi),(Nae,Cc,YZj),(Nae,Cc,YZk),(Nae,Cc,YZl),( Nae,Cc,YZm),(Nae,Cc,YZn),(Nae,Cc,YZo),(Nae,Cc,YZp),(Nae,Cc,YZq),(Nae,Cc,YZr),(Nae ,Cc,YZs),(Nae,Cc,Ya),(Nae,Cc,YZu),(Nae,Cc,YZy),(Nae,Cc,YZw),(Nae,Cc,YZx),(Nae,Cc, YZy),(Nae,Cc,YZz),(Nae,Cc,YZaa),(Nae,Cc,YZab),(Nae,Cc,YZac),(Nae,Cc,YZad),(Nae,Cc, YZae),(Nae,Cc,YZaf),(Nae,Cc,YZag),(Nae,Cd,YZa),(Nae,Cd,YZb),(Nae,Cd,YZc),(Nae,Cd, YZd),(Nae,Cd,YZe),(Nae,Cd,YZf),(Nae,Cd,YZg),(Nae,Cd,YZh),(Nae,Cd,YZi),(Nae,Cd,YZj) ,(Nae,Cd,YZk),(Nae,Cd,YZl),(Nae,Cd,YZm),(Nae,Cd,YZn),(Nae,Cd,YZo),(Nae,Cd,YZp),( Nae,Cd,YZq),(Nae,Cd,YZr),(Nae,Cd,YZs),(Nae,Cd,YZt),(Nae,Cd,YZu),(Nae,Cd,YZy),(Nae, Cd,YZw),(Nae,Cd,YZx),(Nae,Cd,YZy),(Nae,Cd,YZz),(Nae,Cd,YZaa),(Nae,Cd,YZab),(Nae, Cd,YZac),(Nae,Cd,YZad),(Nae,Cd,YZae),(Nae,Cd,YZaf),(Nae,Cd,YZag),(Nae,Ce,YZa),(Na e,Ce,YZb),(Nae,Ce,YZc),(Nae,Ce,YZd),(Nae,Ce,YZe),(Nae,Ce,YZf),(Nae,Ce,YZg),(Nae,Ce, YZh),(Nae,Ce,YZi),(Nae,Ce,YZj),(Nae,Ce,YZk),(Nae,Ce,YZl),(Nae,Ce,YZm),(Nae,Ce,YZn) ,(Nae,Ce,YZo),(Nae,Ce,YZp),(Nae,Ce,YZq),(Nae,Ce,YZr),(Nae,Ce,YZs),(Nae,Ce,YZt),(Nae ,Ce,YZu),(Nae,Ce,YZv),(Nae,Ce,YZw),(Nae,Ce,YZx),(Nae,Ce,YZy),(Nae,Ce,YZz),(Nae,Ce, YZaa),(Nae,Ce,YZab),(Nae,Ce,YZac),(Nae,Ce,YZad),(Nae,Ce,YZae),(Nae,Ce,YZaf),(Nae,C e,YZag),(Nae,Cf,YZa),(Nae,Cf,YZb),(Nae,Cf,YZc),(Nae,Cf,YZd),(Nae,Cf,YZe),(Nae,Cf,YZ f),(Nae,Cf,YZg),(Nae,Cf,YZh),(Nae,Cf,YZi),(Nae,Cf,YZj),(Nae,Cf,YZk),(Nae,Cf,YZl),(Nae, Cf,YZm),(Nae,Cf,YZn),(Nae,Cf,YZo),(Nae,Cf,YZp),(Nae,Cf,YZq),(Nae,Cf,YZr),(Nae,Cf,Y Zs),(Nae,Cf,YZt),(Nae,Cf,YZu),(Nae,Cf,YZy),(Nae,Cf,YZw),(Nae,Cf,YZx),(Nae,Cf,YZy),( Nae,Cf,YZz),(Nae,Cf,YZaa),(Nae,Cf,YZab),(Nae,Cf,YZac),(Nae,Cf,YZad),(Nae,Cf,YZae),( Nae,Cf,YZaf),(Nae,Cf,YZag),(Nae,Cg,YZa),(Nae,Cg,YZb),(Nae,Cg,YZc),(Nae,Cg,YZd),(Na e,Cg,YZe),(Nae,Cg,YZf),(Nae,Cg,YZg),(Nae,Cg,YZh),(Nae,Cg,YZi),(Nae,Cg,YZj),(Nae,Cg, YZk),(Nae,Cg,YZl),(Nae,Cg,YZm),(Nae,Cg,YZn),(Nae,Cg,YZo),(Nae,Cg,YZp),(Nae,Cg,YZ q),(Nae,Cg,YZr),(Nae,Cg,YZs),(Nae,Cg,YZt),(Nae,Cg,YZu),(Nae,Cg,YZv),(Nae,Cg,YZw),( Nae,Cg,YZx),(Nae,Cg,YZy),(Nae,Cg,YZz),(Nae,Cg,YZaa),(Nae,Cg,YZab),(Nae,Cg,YZac),( Nae,Cg,YZad),(Nae,Cg,YZae),(Nae,Cg,YZaf),(Nae,Cg,YZag),(Nae,Ch,YZa),(Nae,Ch,YZb), (Nae,Ch,YZc),(Nae,Ch,YZd),(Nae,Ch,YZe),(Nae,Ch,YZf),(Nae,Ch,YZg),(Nae,Ch,YZh),(Na e,Ch,YZi),(Nae,Ch,YZj),(Nae,Ch,YZk),(Nae,Ch,YZl),(Nae,Ch,YZm),(Nae,Ch,YZn),(Nae,C h,YZo),(Nae,Ch,YZp),(Nae,Ch,YZq),(Nae,Ch,YZr),(Nae,Ch,YZs),(Nae,Ch,YZt),(Nae,Ch,Y Zu),(Nae,Ch,YZy),(Nae,Ch,YZw),(Nae,Ch,YZx),(Nae,Ch,YZy),(Nae,Ch,YZz),(Nae,Ch,YZa a),(Nae,Ch,YZab),(Nae,Ch,YZac),(Nae,Ch,YZad),(Nae,Ch,YZae),(Nae,Ch,YZaf),(Nae,Ch,Y Zag),(Nae,Ci,YZa),(Nae,Ci,YZb),(Nae,Ci,YZc),(Nae,Ci,YZd),(Nae,Ci,YZe),(Nae,Ci,YZf),( Nae,Ci,YZg),(Nae,Ci,YZh),(Nae,Ci,YZi),(Nae,Ci,YZj),(Nae,Ci,YZk),(Nae,Ci,YZl),(Nae,Ci, YZm),(Nae,Ci,YZn),(Nae,Ci,YZo),(Nae,Ci,YZp),(Nae,Ci,YZq),(Nae,Ci,YZr),(Nae,Ci,YZs),( Nae,Ci,YZt),(Nae,Ci,YZu),(Nae,Ci,YZy),(Nae,Ci,YZw),(Nae,Ci,YZx),(Nae,Ci,YZy),(Nae,C i,YZz),(Nae,Ci,YZaa),(Nae,Ci,YZab),(Nae,Ci,YZac),(Nae,Ci,YZad),(Nae,Ci,YZae),(Nae,Ci, YZaf),(Nae,Ci,YZag),(Nae,Cj,YZa),(Nae,Cj,YZb),(Nae,Cj,YZc),(Nae,Cj,YZd),(Nae,Cj,YZe) ,(Nae,Cj,YZf),(Nae,Cj,YZg),(Nae,Cj,YZh),(Nae,Cj,YZi),(Nae,Cj,YZj),(Nae,Cj,YZk),(Nae,Cj ,YZl),(Nae,Cj,YZm),(Nae,Cj,YZn),(Nae,Cj,YZo),(Nae,Cj,YZp),(Nae,Cj,YZq),(Nae,Cj,YZr), (Nae,Cj,YZs),(Nae,Cj,YZt),(Nae,Cj,YZu),(Nae,Cj,YZy),(Nae,Cj,YZw),(Nae,Cj,YZx),(Nae,C j,YZy),(Nae,Cj,YZz),(Nae,Cj,YZaa),(Nae,Cj,YZab),(Nae,Cj,YZac),(Nae,Cj,YZad),(Nae,Cj, YZae),(Nae,Cj,YZaf),(Nae,Cj,YZag),(Naf,Ca,YZa),(Naf,Ca,YZb),(Naf,Ca,YZc),(Naf,Ca,YZ d),(Naf,Ca,YZe),(Naf,Ca,YZf),(Naf,Ca,YZg),(Naf,Ca,YZh),(Naf,Ca,YZi),(Naf,Ca,YZj),(Naf ,Ca,YZk),(Naf,Ca,YZl),(Naf,Ca,YZm),(Naf,Ca,YZn),(Naf,Ca,YZo),(Naf,Ca,YZp),(Naf,Ca,Y Zq),(Naf,Ca,YZr),(Naf,Ca,YZs),(Naf,Ca,YZt),(Naf,Ca,YZu),(Naf,Ca,YZy),(Naf,Ca,YZw),( Naf,Ca,YZx),(Naf,Ca,YZy),(Naf,Ca,YZz),(Naf,Ca,YZaa),(Naf,Ca,YZab),(Naf,Ca,YZac),(Na f,Ca,YZad),(Naf,Ca,YZae),(Naf,Ca,YZaf),(Naf,Ca,YZag),(Naf,Cb,YZa),(Naf,Cb,YZb),(Naf, Cb,YZc),(Naf,Cb,YZd),(Naf,Cb,YZe),(Naf,Cb,Ya),(Naf,Cb,YZg),(Naf,Cb,YZh),(Naf,Cb,Y Zi),(Naf,Cb,YZj),(Naf,Cb,YZk),(Naf,Cb,YZl),(Naf,Cb,YZm),(Naf,Cb,YZn),(Naf,Cb,YZo),( Naf,Cb,YZp),(Naf,Cb,YZq),(Naf,Cb,YZr),(Naf,Cb,YZs),(Naf,Cb,YZt),(Naf,Cb,YZu),(Naf,C b,YZv),(Naf,Cb,YZw),(Naf,Cb,YZx),(Naf,Cb,YZy),(Naf,Cb,YZz),(Naf,Cb,YZaa),(Naf,Cb,Y Zab),(Naf,Cb,YZac),(Naf,Cb,YZad),(Naf,Cb,YZae),(Naf,Cb,YZaf),(Naf,Cb,YZag),(Naf,Cc, YZa),(Naf,Cc,YZb),(Naf,Cc,YZc),(Naf,Cc,YZd),(Naf,Cc,YZe),(Naf,Cc,YZf),(Naf,Cc,YZg),( Naf,Cc,YZh),(Naf,Cc,YZi),(Naf,Cc,YZj),(Naf,Cc,YZk),(Naf,Cc,YZl),(Naf,Cc,YZm),(Naf,C c,YZn),(Naf,Cc,YZo),(Naf,Cc,YZp),(Naf,Cc,YZq),(Naf,Cc,YZr),(Naf,Cc,YZs),(Naf,Cc,YZt) ,(Naf,Cc,YZu),(Naf,Cc,YZy),(Naf,Cc,YZw),(Naf,Cc,YZx),(Naf,Cc,YZy),(Naf,Cc,YZz),(Naf ,Cc,YZaa),(Naf,Cc,YZab),(Naf,Cc,YZac),(Naf,Cc,YZad),(Naf,Cc,YZae),(Naf,Cc,YZaf),(Naf ,Cc,YZag),(Naf,Cd,YZa),(Naf,Cd,YZb),(Naf,Cd,YZc),(Naf,Cd,YZd),(Naf,Cd,YZe),(Naf,Cd, YZf),(Naf,Cd,YZg),(Naf,Cd,YZh),(Naf,Cd,YZi),(Naf,Cd,YZj),(Naf,Cd,YZk),(Naf,Cd,YZl),( Naf,Cd,YZm),(Naf,Cd,YZn),(Naf,Cd,YZo),(Naf,Cd,YZp),(Naf,Cd,YZq),(Naf,Cd,YZr),(Naf, Cd,YZs),(Naf,Cd,YZt),(Naf,Cd,YZu),(Naf,Cd,YZy),(Naf,Cd,YZw),(Naf,Cd,YZx),(Naf,Cd,Y Zy),(Naf,Cd,YZz),(Naf,Cd,YZaa),(Naf,Cd,YZab),(Naf,Cd,YZac),(Naf,Cd,YZad),(Naf,Cd,Y Zae),(Naf,Cd,YZaf),(Naf,Cd,YZag),(Naf,Ce,YZa),(Naf,Ce,YZb),(Naf,Ce,YZc),(Naf,Ce,Yal) ,(Naf,Ce,YZe),(Naf,Ce,YZf),(Naf,Ce,YZg),(Naf,Ce,YZh),(Naf,Ce,YZi),(Naf,Ce,YZj),(Naf, Ce,YZk),(Naf,Ce,YZl),(Naf,Ce,YZm),(Naf,Ce,YZn),(Naf,Ce,YZo),(Naf,Ce,YZp),(Naf,Ce,Y Zq),(Naf,Ce,YZr),(Naf,Ce,YZs),(Naf,Ce,YZf),(Naf,Ce,YZu),(Naf,Ce,YZy),(Naf,Ce,YZw),( Naf,Ce,YZx),(Naf,Ce,YZy),(Naf,Ce,YZz),(Naf,Ce,YZaa),(Naf,Ce,YZab),(Naf,Ce,YZac),(Na f,Ce,YZad),(Naf,Ce,YZae),(Naf,Ce,YZaf),(Naf,Ce,YZag),(Naf,Cf,YZa),(Naf,Cf,YZb),(Naf, Cf,YZc),(Naf,Cf,YZd),(Naf,Cf,YZe),(Naf,Cf,YZf),(Naf,Cf,YZg),(Naf,Cf,YZh),(Naf,Cf,YZi) ,(Naf,Cf,YZj),(Naf,Cf,YZk),(Naf,Cf,YZl),(Naf,Cf,YZm),(Naf,Cf,YZn),(Naf,Cf,YZo),(Naf,C f,YZp),(Naf,Cf,YZq),(Naf,Cf,YZr),(Naf,Cf,YZs),(Naf,Cf,YZt),(Naf,Cf,YZu),(Naf,Cf,YZy),( Naf,Cf,YZw),(Naf,Cf,YZx),(Naf,Cf,YZy),(Naf,Cf,YZz),(Naf,Cf,YZaa),(Naf,Cf,YZab),(Naf, Cf,YZac),(Naf,Cf,YZad),(Naf,Cf,YZae),(Naf,Cf,YZaf),(Naf,Cf,YZag),(Naf,Cg,YZa),(Naf,C g,YZb),(Naf,Cg,YZc),(Naf,Cg,YZd),(Naf,Cg,YZe),(Naf,Cg,YZf),(Naf,Cg,YZg),(Naf,Cg,YZ h),(Naf,Cg,YZi),(Naf,Cg,YZj),(Naf,Cg,YZk),(Naf,Cg,YZl),(Naf,Cg,YZm),(Naf,Cg,YZn),(N af,Cg,YZo),(Naf,Cg,YZp),(Naf,Cg,YZq),(Naf,Cg,YZr),(Naf,Cg,YZs),(Naf,Cg,YZt),(Naf,Cg, YZu),(Naf,Cg,YZy),(Naf,Cg,YZw),(Naf,Cg,YZx),(Naf,Cg,YZy),(Naf,Cg,YZz),(Naf,Cg,YZa a),(Naf,Cg,YZab),(Naf,Cg,YZac),(Naf,Cg,YZad),(Naf,Cg,YZae),(Naf,Cg,YZaf),(Naf,Cg,YZ ag),(Naf,Ch,YZa),(Naf,Ch,YZb),(Naf,Ch,YZc),(Naf,Ch,YZd),(Naf,Ch,YZe),(Naf,Ch,YZf),( Naf,Ch,YZg),(Naf,Ch,YZh),(Naf,Ch,YZi),(Naf,Ch,YZj),(Naf,Ch,YZk),(Naf,Ch,YZl),(Naf,C h,YZm),(Naf,Ch,YZn),(Naf,Ch,YZo),(Naf,Ch,YZp),(Naf,Ch,YZq),(Naf,Ch,YZr),(Naf,Ch,Y Zs),(Naf,Ch,YZf),(Naf,Ch,YZu),(Naf,Ch,YZv),(Naf,Ch,YZw),(Naf,Ch,YZx),(Naf,Ch,YZy),( Naf,Ch,YZz),(Naf,Ch,YZaa),(Naf,Ch,YZab),(Naf,Ch,YZac),(Naf,Ch,YZad),(Naf,Ch,YZae),( Nat,Ch,YZaf),(Naf,Ch,YZag),(Naf,Ci,YZa),(Naf,Ci,YZb),(Naf,Ci,YZc),(Naf,Ci,YZd),(Naf, Ci,YZe),(Naf,Ci,YZf),(Naf,Ci,YZg),(Naf,Ci,YZh),(Naf,Ci,YZi),(Naf,Ci,YZj),(Naf,Ci,YZk),( Naf,Ci,YZl),(Naf,Ci,YZm),(Naf,Ci,YZn),(Naf,Ci,YZo),(Naf,Ci,YZp),(Naf,Ci,YZq),(Naf,Ci, YZr),(Naf,Ci,YZs),(Naf,Ci,YZt),(Naf,Ci,YZu),(Naf,Ci,YZy),(Naf,Ci,YZw),(Naf,Ci,YZx),(N af,Ci,YZy),(Naf,Ci,YZz),(Naf,Ci,YZaa),(Naf,Ci,YZab),(Naf,Ci,YZac),(Naf,Ci,YZad),(Naf,C i,YZae),(Naf,Ci,YZaf),(Naf,Ci,YZag),(Naf,Cj,YZa),(Naf,Cj,YZb),(Naf,Cj,YZc),(Naf,Cj,Yal) ,(Naf,Cj,YZe),(Naf,Cj,YZf),(Naf,Cj,YZg),(Naf,Cj,YZh),(Naf,Cj,YZi),(Naf,Cj,YZj),(Naf,Cj, YZk),(Naf,Cj,YZl),(Naf,Cj,YZm),(Naf,Cj,YZn),(Naf,Cj,YZo),(Naf,Cj,YZp),(Naf,Cj,YZq),( Naf,Cj,YZr),(Naf,Cj,YZs),(Naf,Cj,YZt),(Naf,Cj,YZu),(Naf,Cj,YZy),(Naf,Cj,YZw),(Naf,Cj,Y Zx),(Naf,Cj,YZy),(Naf,Cj,YZz),(Naf,Cj,YZaa),(Naf,Cj,YZab),(Naf,Cj,YZac),(Naf,Cj,YZad), (Naf,Cj,YZae),(Naf,Cj,YZaf),(Naf,Cj,YZag),(Nag,Ca,YZa),(Nag,Ca,YZb),(Nag,Ca,YZc),(N ag,Ca,YZd),(Nag,Ca,YZe),(Nag,Ca,YZf),(Nag,Ca,YZg),(Nag,Ca,YZh),(Nag,Ca,YZi),(Nag,C a,YZj),(Nag,Ca,YZk),(Nag,Ca,YZl),(Nag,Ca,YZm),(Nag,Ca,YZn),(Nag,Ca,YZo),(Nag,Ca,Y Zp),(Nag,Ca,YZq),(Nag,Ca,YZr),(Nag,Ca,YZs),(Nag,Ca,YZt),(Nag,Ca,YZu),(Nag,Ca,YZy), (Nag,Ca,YZw),(Nag,Ca,YZx),(Nag,Ca,YZy),(Nag,Ca,YZz),(Nag,Ca,YZaa),(Nag,Ca,YZab),( Nag,Ca,YZac),(Nag,Ca,YZad),(Nag,Ca,YZae),(Nag,Ca,YZaf),(Nag,Ca,YZag),(Nag,Cb,YZa) ,(Nag,Cb,YZb),(Nag,Cb,YZc),(Nag,Cb,YZd),(Nag,Cb,YZe),(Nag,Cb,YZf),(Nag,Cb,YZg),(N ag,Cb,YZh),(Nag,Cb,YZi),(Nag,Cb,YZj),(Nag,Cb,YZk),(Nag,Cb,YZl),(Nag,Cb,YZm),(Nag, Cb,YZn),(Nag,Cb,YZo),(Nag,Cb,YZp),(Nag,Cb,YZq),(Nag,Cb,YZr),(Nag,Cb,YZs),(Nag,Cb ,YZt),(Nag,Cb,YZu),(Nag,Cb,YZy),(Nag,Cb,YZw),(Nag,Cb,YZx),(Nag,Cb,YZy),(Nag,Cb,Y Zz),(Nag,Cb,YZaa),(Nag,Cb,YZab),(Nag,Cb,YZac),(Nag,Cb,YZad),(Nag,Cb,YZae),(Nag,Cb ,YZaf),(Nag,Cb,YZag),(Nag,Cc,YZa),(Nag,Cc,YZb),(Nag,Cc,YZc),(Nag,Cc,YZd),(Nag,Cc, YZe),(Nag,Cc,YZf),(Nag,Cc,YZg),(Nag,Cc,Ya),(Nag,Cc,YZi),(Nag,Cc,YZj),(Nag,Cc,YZk) ,(Nag,Cc,YZl),(Nag,Cc,YZm),(Nag,Cc,YZn),(Nag,Cc,YZo),(Nag,Cc,YZp),(Nag,Cc,YZq),( Nag,Cc,YZr),(Nag,Cc,YZs),(Nag,Cc,YZf),(Nag,Cc,YZu),(Nag,Cc,YZy),(Nag,Cc,YZw),(Nag ,Cc,YZx),(Nag,Cc,YZy),(Nag,Cc,YZz),(Nag,Cc,YZaa),(Nag,Cc,YZab),(Nag,Cc,YZac),(Nag, Cc,YZad),(Nag,Cc,YZae),(Nag,Cc,YZaf),(Nag,Cc,YZag),(Nag,Cd,YZa),(Nag,Cd,YZb),(Nag ,Cd,YZc),(Nag,Cd,YZd),(Nag,Cd,YZe),(Nag,Cd,YZf),(Nag,Cd,YZg),(Nag,Cd,YZh),(Nag,C d,YZi),(Nag,Cd,YZj),(Nag,Cd,YZk),(Nag,Cd,YZl),(Nag,Cd,YZm),(Nag,Cd,YZn),(Nag,Cd,Y Zo),(Nag,Cd,YZp),(Nag,Cd,YZq),(Nag,Cd,YZr),(Nag,Cd,YZs),(Nag,Cd,YZt),(Nag,Cd,YZu) ,(Nag,Cd,YZv),(Nag,Cd,YZw),(Nag,Cd,YZx),(Nag,Cd,YZy),(Nag,Cd,YZz),(Nag,Cd,YZaa), (Nag,Cd,YZab),(Nag,Cd,YZac),(Nag,Cd,YZad),(Nag,Cd,YZae),(Nag,Cd,YZaf),(Nag,Cd,YZ ag),(Nag,Ce,YZa),(Nag,Ce,YZb),(Nag,Ce,YZc),(Nag,Ce,YZd),(Nag,Ce,YZe),(Nag,Ce,YZf), (Nag,Ce,YZg),(Nag,Ce,YZh),(Nag,Ce,YZi),(Nag,Ce,YZj),(Nag,Ce,YZk),(Nag,Ce,YZl),(Nag ,Ce,YZm),(Nag,Ce,YZn),(Nag,Ce,YZo),(Nag,Ce,YZp),(Nag,Ce,YZq),(Nag,Ce,YZr),(Nag,C e,YZs),(Nag,Ce,YZf),(Nag,Ce,YZu),(Nag,Ce,YZy),(Nag,Ce,YZw),(Nag,Ce,YZx),(Nag,Ce,Y Zy),(Nag,Ce,YZz),(Nag,Ce,YZaa),(Nag,Ce,YZab),(Nag,Ce,YZac),(Nag,Ce,YZad),(Nag,Ce, YZae),(Nag,Ce,YZaf),(Nag,Ce,YZag),(Nag,Cf,YZa),(Nag,Cf,YZb),(Nag,Cf,YZc),(Nag,Cf,Y Zd),(Nag,Cf,YZe),(Nag,Cf,YZf),(Nag,Cf,YZg),(Nag,Cf,YZh),(Nag,Cf,YZi),(Nag,Cf,YZj),(N ag,Cf,YZk),(Nag,Cf,YZl),(Nag,Cf,YZm),(Nag,Cf,YZn),(Nag,Cf,YZo),(Nag,Cf,YZp),(Nag,C f,YZq),(Nag,Cf,YZr),(Nag,Cf,YZs),(Nag,Cf,YZf),(Nag,Cf,YZu),(Nag,Cf,YZy),(Nag,Cf,YZ w),(Nag,Cf,YZx),(Nag,Cf,YZy),(Nag,Cf,YZz),(Nag,Cf,YZaa),(Nag,Cf,YZab),(Nag,Cf,YZac) ,(Nag,Cf,YZad),(Nag,Cf,YZae),(Nag,Cf,YZaf),(Nag,Cf,YZag),(Nag,Cg,YZa),(Nag,Cg,YZb) ,(Nag,Cg,YZc),(Nag,Cg,YZd),(Nag,Cg,YZe),(Nag,Cg,YZf),(Nag,Cg,YZg),(Nag,Cg,YZh),( Nag,Cg,YZi),(Nag,Cg,YZj),(Nag,Cg,YZk),(Nag,Cg,YZl),(Nag,Cg,YZm),(Nag,Cg,YZn),(Na g,Cg,YZo),(Nag,Cg,YZp),(Nag,Cg,YZq),(Nag,Cg,YZr),(Nag,Cg,YZs),(Nag,Cg,YZt),(Nag,C g,YZu),(Nag,Cg,YZy),(Nag,Cg,YZw),(Nag,Cg,YZx),(Nag,Cg,YZy),(Nag,Cg,YZz),(Nag,Cg, YZaa),(Nag,Cg,YZab),(Nag,Cg,YZac),(Nag,Cg,YZad),(Nag,Cg,YZae),(Nag,Cg,YZaf),(Nag, Cg,YZag),(Nag,Ch,YZa),(Nag,Ch,YZb),(Nag,Ch,YZc),(Nag,Ch,YZd),(Nag,Ch,YZe),(Nag,C h,YZf),(Nag,Ch,YZg),(Nag,Ch,YZh),(Nag,Ch,YZi),(Nag,Ch,YZj),(Nag,Ch,YZk),(Nag,Ch,Y Zl),(Nag,Ch,YZm),(Nag,Ch,YZn),(Nag,Ch,YZo),(Nag,Ch,YZp),(Nag,Ch,YZq),(Nag,Ch,YZr) ,(Nag,Ch,YZs),(Nag,Ch,YZt),(Nag,Ch,YZu),(Nag,Ch,YZy),(Nag,Ch,YZw),(Nag,Ch,YZx),( Nag,Ch,YZy),(Nag,Ch,YZz),(Nag,Ch,YZaa),(Nag,Ch,YZab),(Nag,Ch,YZac),(Nag,Ch,YZad) ,(Nag,Ch,YZae),(Nag,Ch,YZaf),(Nag,Ch,YZag),(Nag,Ci,YZa),(Nag,Ci,YZb),(Nag,Ci,YZc),( Nag,Ci,YZd),(Nag,Ci,YZe),(Nag,Ci,YZf),(Nag,Ci,YZg),(Nag,Ci,YZh),(Nag,Ci,YZi),(Nag,Ci ,YZj),(Nag,Ci,YZk),(Nag,Ci,YZl),(Nag,Ci,YZm),(Nag,Ci,YZn),(Nag,Ci,YZo),(Nag,Ci,YZp) ,(Nag,Ci,YZq),(Nag,Ci,YZr),(Nag,Ci,YZs),(Nag,Ci,YZt),(Nag,Ci,YZu),(Nag,Ci,YZv),(Nag, Ci,YZw),(Nag,Ci,YZx),(Nag,Ci,YZy),(Nag,Ci,YZz),(Nag,Ci,YZaa),(Nag,Ci,YZab),(Nag,Ci, YZac),(Nag,Ci,YZad),(Nag,Ci,YZae),(Nag,Ci,YZaf),(Nag,Ci,YZag),(Nag,Cj,YZa),(Nag,Cj, YZb),(Nag,Cj,YZc),(Nag,Cj,YZd),(Nag,Cj,YZe),(Nag,Cj,YZf),(Nag,Cj,YZg),(Nag,Cj,YZh), (Nag,Cj,YZi),(Nag,Cj,YZj),(Nag,Cj,YZk),(Nag,Cj,YZl),(Nag,Cj,YZm),(Nag,Cj,YZn),(Nag, Cj,YZo),(Nag,Cj,YZp),(Nag,Cj,YZq),(Nag,Cj,YZr),(Nag,Cj,YZs),(Nag,Cj,YZt),(Nag,Cj,YZ u),(Nag,Cj,YZv),(Nag,Cj,YZw),(Nag,Cj,YZx),(Nag,Cj,YZy),(Nag,Cj,YZz),(Nag,Cj,YZaa),( Nag,Cj,YZab),(Nag,Cj,YZac),(Nag,Cj,YZad),(Nag,Cj,YZae),(Nag,Cj,YZaf),(Nag,Cj,YZag),( Nah,Ca,YZa),(Nah,Ca,YZb),(Nah,Ca,YZc),(Nah,Ca,YZd),(Nah,Ca,YZe),(Nah,Ca,YZf),(Nah ,Ca,YZg),(Nah,Ca,YZh),(Nah,Ca,YZi),(Nah,Ca,YZj),(Nah,Ca,YZk),(Nah,Ca,YZl),(Nah,Ca, YZm),(Nah,Ca,YZn),(Nah,Ca,YZo),(Nah,Ca,YZp),(Nah,Ca,YZq),(Nah,Ca,YZr),(Nah,Ca,YZ s),(Nah,Ca,YZt),(Nah,Ca,YZu),(Nah,Ca,YZv),(Nah,Ca,YZw),(Nah,Ca,YZx),(Nah,Ca,YZy),( Nah,Ca,YZz),(Nah,Ca,YZaa),(Nah,Ca,YZab),(Nah,Ca,YZac),(Nah,Ca,YZad),(Nah,Ca,YZae) ,(Nah,Ca,YZaf),(Nah,Ca,YZag),(Nah,Cb,YZa),(Nah,Cb,YZb),(Nah,Cb,YZc),(Nah,Cb,YZd),( Nah,Cb,YZe),(Nah,Cb,YZf),(Nah,Cb,YZg),(Nah,Cb,YM),(Nah,Cb,YZi),(Nah,Cb,YZj),(Nah ,Cb,YZk),(Nah,Cb,YZl),(Nah,Cb,YZm),(Nah,Cb,YZn),(Nah,Cb,YZo),(Nah,Cb,YZp),(Nah,C b,YZq),(Nah,Cb,YZr),(Nah,Cb,YZs),(Nah,Cb,YZt),(Nah,Cb,YZu),(Nah,Cb,YZv),(Nah,Cb,Y Zw),(Nah,Cb,YZx),(Nah,Cb,YZy),(Nah,Cb,YZz),(Nah,Cb,YZaa),(Nah,Cb,YZab),(Nah,Cb,Y Zac),(Nah,Cb,YZad),(Nah,Cb,YZae),(Nah,Cb,YZaf),(Nah,Cb,YZag),(Nah,Cc,YZa),(Nah,Cc, YZb),(Nah,Cc,YZc),(Nah,Cc,YZd),(Nah,Cc,YZe),(Nah,Cc,YZf),(Nah,Cc,YZg),(Nah,Cc,YZ h),(Nah,Cc,YZi),(Nah,Cc,YZj),(Nah,Cc,YZk),(Nah,Cc,YZl),(Nah,Cc,YZm),(Nah,Cc,YZn),( Nah,Cc,YZo),(Nah,Cc,YZp),(Nah,Cc,YZq),(Nah,Cc,YZr),(Nah,Cc,YZs),(Nah,Cc,YZt),(Nah, Cc,YZu),(Nah,Cc,YZv),(Nah,Cc,YZw),(Nah,Cc,YZx),(Nah,Cc,YZy),(Nah,Cc,YZz),(Nah,Cc ,YZaa),(Nah,Cc,YZab),(Nah,Cc,YZac),(Nah,Cc,YZad),(Nah,Cc,YZae),(Nah,Cc,YZaf),(Nah, Cc,YZag),(Nah,Cd,YZa),(Nah,Cd,YZb),(Nah,Cd,YZc),(Nah,Cd,Yal),(Nah,Cd,YZe),(Nah,C d,YZf),(Nah,Cd,YZg),(Nah,Cd,YZh),(Nah,Cd,YZi),(Nah,Cd,YZj),(Nah,Cd,YZk),(Nah,Cd,Y Zl),(Nah,Cd,YZm),(Nah,Cd,YZn),(Nah,Cd,YZo),(Nah,Cd,YZp),(Nah,Cd,YZq),(Nah,Cd,YZr) ,(Nah,Cd,YZs),(Nah,Cd,YZt),(Nah,Cd,YZu),(Nah,Cd,YZv),(Nah,Cd,YZw),(Nah,Cd,YZx),( Nah,Cd,YZy),(Nah,Cd,YZz),(Nah,Cd,YZaa),(Nah,Cd,YZab),(Nah,Cd,YZac),(Nah,Cd,YZad) ,(Nah,Cd,YZae),(Nah,Cd,YZaf),(Nah,Cd,YZag),(Nah,Ce,YZa),(Nah,Ce,YZb),(Nah,Ce,YZc), (Nah,Ce,YZd),(Nah,Ce,YZe),(Nah,Ce,YZf),(Nah,Ce,YZg),(Nah,Ce,YZh),(Nah,Ce,YZi),(Na h,Ce,YZj),(Nah,Ce,YZk),(Nah,Ce,YZl),(Nah,Ce,YZm),(Nah,Ce,YZn),(Nah,Ce,YZo),(Nah,C e,YZp),(Nah,Ce,YZq),(Nah,Ce,YZr),(Nah,Ce,YZs),(Nah,Ce,YZt),(Nah,Ce,YZu),(Nah,Ce,Y Zv),(Nah,Ce,YZw),(Nah,Ce,YZx),(Nah,Ce,YZy),(Nah,Ce,YZz),(Nah,Ce,YZaa),(Nah,Ce,YZ ab),(Nah,Ce,YZac),(Nah,Ce,YZad),(Nah,Ce,YZae),(Nah,Ce,YZaf),(Nah,Ce,YZag),(Nah,Cf, YZa),(Nah,Cf,YZb),(Nah,Cf,YZc),(Nah,Cf,YZd),(Nah,Cf,YZe),(Nah,Cf,YZf),(Nah,Cf,YZg), (Nah,Cf,YZh),(Nah,Cf,YZi),(Nah,Cf,YZj),(Nah,Cf,YZk),(Nah,Cf,YZl),(Nah,Cf,YZm),(Nah, Cf,YZn),(Nah,Cf,YZo),(Nah,Cf,YZp),(Nah,Cf,YZq),(Nah,Cf,YZr),(Nah,Cf,YZs),(Nah,Cf,Y Zt),(Nah,Cf,YZu),(Nah,Cf,YZy),(Nah,Cf,YZw),(Nah,Cf,YZx),(Nah,Cf,YZy),(Nah,Cf,YZz),( Nah,Cf,YZaa),(Nah,Cf,YZab),(Nah,Cf,YZac),(Nah,Cf,YZad),(Nah,Cf,YZae),(Nah,Cf,YZaf), (Nah,Cf,YZag),(Nah,Cg,YZa),(Nah,Cg,YZb),(Nah,Cg,YZc),(Nah,Cg,YZd),(Nah,Cg,YZe),( Nah,Cg,YZf),(Nah,Cg,YZg),(Nah,Cg,YZh),(Nah,Cg,YZi),(Nah,Cg,YZj),(Nah,Cg,YZk),(Nah ,Cg,YZl),(Nah,Cg,YZm),(Nah,Cg,YZn),(Nah,Cg,YZo),(Nah,Cg,YZp),(Nah,Cg,YZq),(Nah,C g,YZr),(Nah,Cg,YZs),(Nah,Cg,YZf),(Nah,Cg,YZu),(Nah,Cg,YZy),(Nah,Cg,YZw),(Nah,Cg, YZx),(Nah,Cg,YZy),(Nah,Cg,YZz),(Nah,Cg,YZaa),(Nah,Cg,YZab),(Nah,Cg,YZac),(Nah,Cg ,YZad),(Nah,Cg,YZae),(Nah,Cg,YZaf),(Nah,Cg,YZag),(Nah,Ch,YZa),(Nah,Ch,YZb),(Nah,C h,YZc),(Nah,Ch,YZd),(Nah,Ch,YZe),(Nah,Ch,YZf),(Nah,Ch,YZg),(Nah,Ch,YZh),(Nah,Ch, YZi),(Nah,Ch,YZj),(Nah,Ch,YZk),(Nah,Ch,YZl),(Nah,Ch,YZm),(Nah,Ch,YZn),(Nah,Ch,YZ o),(Nah,Ch,YZp),(Nah,Ch,YZq),(Nah,Ch,YZr),(Nah,Ch,YZs),(Nah,Ch,YZt),(Nah,Ch,YZu),( Nah,Ch,YZy),(Nah,Ch,YZw),(Nah,Ch,YZx),(Nah,Ch,YZy),(Nah,Ch,YZz),(Nah,Ch,YZaa),( Nah,Ch,YZab),(Nah,Ch,YZac),(Nah,Ch,YZad),(Nah,Ch,YZae),(Nah,Ch,YZaf),(Nah,Ch,YZa g),(Nah,Ci,YZa),(Nah,Ci,YZb),(Nah,Ci,YZc),(Nah,Ci,YZd),(Nah,Ci,YZe),(Nah,Ci,YZf),(Na h,Ci,YZg),(Nah,Ci,YZh),(Nah,Ci,YZi),(Nah,Ci,YZj),(Nah,Ci,YZk),(Nah,Ci,YZl),(Nah,Ci,Y Zm),(Nah,Ci,YZn),(Nah,Ci,YZo),(Nah,Ci,YZp),(Nah,Ci,YZq),(Nah,Ci,YZr),(Nah,Ci,YZs),( Nah,Ci,YZf),(Nah,Ci,YZu),(Nah,Ci,YZy),(Nah,Ci,YZw),(Nah,Ci,YZx),(Nah,Ci,YZy),(Nah, Ci,YZz),(Nah,Ci,YZaa),(Nah,Ci,YZab),(Nah,Ci,YZac),(Nah,Ci,YZad),(Nah,Ci,YZae),(Nah, Ci,YZaf),(Nah,Ci,YZag),(Nah,Cj,YZa),(Nah,Cj,YZb),(Nah,Cj,YZc),(Nah,Cj,YZd),(Nah,Cj, YZe),(Nah,Cj,YZf),(Nah,Cj,YZg),(Nah,Cj,YZh),(Nah,Cj,YZi),(Nah,Cj,YZj),(Nah,Cj,YZk),( Nah,Cj,YZl),(Nah,Cj,YZm),(Nah,Cj,YZn),(Nah,Cj,YZo),(Nah,Cj,YZp),(Nah,Cj,YZq),(Nah, Cj,YZr),(Nah,Cj,YZs),(Nah,Cj,YZt),(Nah,Cj,YZu),(Nah,Cj,YZy),(Nah,Cj,YZw),(Nah,Cj,YZ x),(Nah,Cj,YZy),(Nah,Cj,YZz),(Nah,Cj,YZaa),(Nah,Cj,YZab),(Nah,Cj,YZac),(Nah,Cj,YZad) ,(Nah,Cj,YZae),(Nah,Cj,YZaf),(Nah,Cj,YZag),(Nai,Ca,YZa),(Nai,Ca,YZb),(Nai,Ca,YZc),(N ai,Ca,YZcl),(Nai,Ca,YZe),(Nai,Ca,YZf),(Nai,Ca,YZg),(Nai,Ca,Ya),(Nai,Ca,YZi),(Nai,Ca,Y Zj),(Nai,Ca,YZk),(Nai,Ca,YZl),(Nai,Ca,YZm),(Nai,Ca,YZn),(Nai,Ca,YZo),(Nai,Ca,YZp),(N ai,Ca,YZq),(Nai,Ca,YZr),(Nai,Ca,YZs),(Nai,Ca,YZf),(Nai,Ca,YZu),(Nai,Ca,YZy),(Nai,Ca,Y Zw),(Nai,Ca,YZx),(Nai,Ca,YZy),(Nai,Ca,YZz),(Nai,Ca,YZaa),(Nai,Ca,YZab),(Nai,Ca,YZac) ,(Nai,Ca,YZad),(Nai,Ca,YZae),(Nai,Ca,YZaf),(Nai,Ca,YZag),(Nai,Cb,YZa),(Nai,Cb,YZb),( Nai,Cb,YZc),(Nai,Cb,YZd),(Nai,Cb,YZe),(Nai,Cb,YZf),(Nai,Cb,YZg),(Nai,Cb,YZh),(Nai,C b,YZi),(Nai,Cb,YZj),(Nai,Cb,YZk),(Nai,Cb,YZl),(Nai,Cb,YZm),(Nai,Cb,YZn),(Nai,Cb,YZo) ,(Nai,Cb,YZp),(Nai,Cb,YZq),(Nai,Cb,YZr),(Nai,Cb,YZs),(Nai,Cb,YZt),(Nai,Cb,YZu),(Nai, Cb,YZy),(Nai,Cb,YZw),(Nai,Cb,YZx),(Nai,Cb,YZy),(Nai,Cb,YZz),(Nai,Cb,YZaa),(Nai,Cb, YZab),(Nai,Cb,YZac),(Nai,Cb,YZad),(Nai,Cb,YZae),(Nai,Cb,YZaf),(Nai,Cb,YZag),(Nai,Cc, YZa),(Nai,Cc,YZb),(Nai,Cc,YZc),(Nai,Cc,YZd),(Nai,Cc,YZe),(Nai,Cc,YZf),(Nai,Cc,YZg),( Nai,Cc,YZh),(Nai,Cc,YZi),(Nai,Cc,YZj),(Nai,Cc,YZk),(Nai,Cc,YZl),(Nai,Cc,YZm),(Nai,Cc, YZn),(Nai,Cc,YZo),(Nai,Cc,YZp),(Nai,Cc,YZq),(Nai,Cc,YZr),(Nai,Cc,YZs),(Nai,Cc,YZt),( Nai,Cc,YZu),(Nai,Cc,YZy),(Nai,Cc,YZw),(Nai,Cc,YZx),(Nai,Cc,YZy),(Nai,Cc,YZz),(Nai,C c,YZaa),(Nai,Cc,YZab),(Nai,Cc,YZac),(Nai,Cc,YZad),(Nai,Cc,YZae),(Nai,Cc,YZaf),(Nai,Cc ,YZag),(Nai,Cd,YZa),(Nai,Cd,YZb),(Nai,Cd,YZc),(Nai,Cd,YZd),(Nai,Cd,YZe),(Nai,Cd,YZf) ,(Nai,Cd,YZg),(Nai,Cd,YZh),(Nai,Cd,YZi),(Nai,Cd,YZj),(Nai,Cd,YZk),(Nai,Cd,YZl),(Nai, Cd,YZm),(Nai,Cd,YZn),(Nai,Cd,YZo),(Nai,Cd,YZp),(Nai,Cd,YZq),(Nai,Cd,YZr),(Nai,Cd,Y Zs),(Nai,Cd,YZt),(Nai,Cd,YZu),(Nai,Cd,YZy),(Nai,Cd,YZw),(Nai,Cd,YZx),(Nai,Cd,YZy),( Nai,Cd,YZz),(Nai,Cd,YZaa),(Nai,Cd,YZab),(Nai,Cd,YZac),(Nai,Cd,YZad),(Nai,Cd,YZae),( Nai,Cd,YZaf),(Nai,Cd,YZag),(Nai,Ce,YZa),(Nai,Ce,YZb),(Nai,Ce,YZc),(Nai,Ce,YZd),(Nai, Ce,YZe),(Nai,Ce,YZf),(Nai,Ce,YZg),(Nai,Ce,YZh),(Nai,Ce,YZi),(Nai,Ce,YZj),(Nai,Ce,YZk) ,(Nai,Ce,YZl),(Nai,Ce,YZm),(Nai,Ce,YZn),(Nai,Ce,YZo),(Nai,Ce,YZp),(Nai,Ce,YZq),(Nai, Ce,YZr),(Nai,Ce,YZs),(Nai,Ce,YZt),(Nai,Ce,YZu),(Nai,Ce,YZy),(Nai,Ce,YZw),(Nai,Ce,YZ x),(Nai,Ce,YZy),(Nai,Ce,YZz),(Nai,Ce,YZaa),(Nai,Ce,YZab),(Nai,Ce,YZac),(Nai,Ce,YZad), (Nai,Ce,YZae),(Nai,Ce,YZaf),(Nai,Ce,YZag),(Nai,Cf,YZa),(Nai,Cf,YZb),(Nai,Cf,YZc),(Nai, Cf,YZd),(Nai,Cf,YZe),(Nai,Cf,YZf),(Nai,Cf,YZg),(Nai,Cf,YZh),(Nai,Cf,YZi),(Nai,Cf,YZj),( Nai,Cf,YZk),(Nai,Cf,YZl),(Nai,Cf,YZm),(Nai,Cf,YZn),(Nai,Cf,YZo),(Nai,Cf,YZp),(Nai,Cf, YZq),(Nai,Cf,YZr),(Nai,Cf,YZs),(Nai,Cf,YZt),(Nai,Cf,YZu),(Nai,Cf,YZy),(Nai,Cf,YZw),(N ai,Cf,YZx),(Nai,Cf,YZy),(Nai,Cf,YZz),(Nai,Cf,YZaa),(Nai,Cf,YZab),(Nai,Cf,YZac),(Nai,Cf, YZad),(Nai,Cf,YZae),(Nai,Cf,YZaf),(Nai,Cf,YZag),(Nai,Cg,YZa),(Nai,Cg,YZb),(Nai,Cg,YZ c),(Nai,Cg,YZd),(Nai,Cg,YZe),(Nai,Cg,YZf),(Nai,Cg,YZg),(Nai,Cg,YZh),(Nai,Cg,YZi),(Nai ,Cg,YZj),(Nai,Cg,YZk),(Nai,Cg,YZl),(Nai,Cg,YZm),(Nai,Cg,YZn),(Nai,Cg,YZo),(Nai,Cg,Y Zp),(Nai,Cg,YZq),(Nai,Cg,YZr),(Nai,Cg,YZs),(Nai,Cg,YZt),(Nai,Cg,YZu),(Nai,Cg,YZy),(N ai,Cg,YZw),(Nai,Cg,YZx),(Nai,Cg,YZy),(Nai,Cg,YZz),(Nai,Cg,YZaa),(Nai,Cg,YZab),(Nai, Cg,YZac),(Nai,Cg,YZad),(Nai,Cg,YZae),(Nai,Cg,YZaf),(Nai,Cg,YZag),(Nai,Ch,YZa),(Nai, Ch,YZb),(Nai,Ch,YZc),(Nai,Ch,YZcl),(Nai,Ch,YZe),(Nai,Ch,YZf),(Nai,Ch,YZg),(Nai,Ch,Y Zh),(Nai,Ch,YZi),(Nai,Ch,YZj),(Nai,Ch,YZk),(Nai,Ch,YZl),(Nai,Ch,YZm),(Nai,Ch,YZn),(N ai,Ch,YZo),(Nai,Ch,YZp),(Nai,Ch,YZq),(Nai,Ch,YZr),(Nai,Ch,YZs),(Nai,Ch,YZt),(Nai,Ch, YZu),(Nai,Ch,YZy),(Nai,Ch,YZw),(Nai,Ch,YZx),(Nai,Ch,YZy),(Nai,Ch,YZz),(Nai,Ch,YZaa) ,(Nai,Ch,YZab),(Nai,Ch,YZac),(Nai,Ch,YZad),(Nai,Ch,YZae),(Nai,Ch,YZaf),(Nai,Ch,YZag) ,(Nai,Ci,YZa),(Nai,Ci,YZb),(Nai,Ci,YZc),(Nai,Ci,YZd),(Nai,Ci,YZe),(Nai,Ci,YZf),(Nai,Ci, YZg),(Nai,Ci,YZh),(Nai,Ci,YZi),(Nai,Ci,YZj),(Nai,Ci,YZk),(Nai,Ci,YZl),(Nai,Ci,YZm),(Na i,Ci,YZn),(Nai,Ci,YZo),(Nai,Ci,YZp),(Nai,Ci,YZq),(Nai,Ci,YZr),(Nai,Ci,YZs),(Nai,Ci,YZt), (Nai,Ci,YZu),(Nai,Ci,YZy),(Nai,Ci,YZw),(Nai,Ci,YZx),(Nai,Ci,YZy),(Nai,Ci,YZz),(Nai,Ci, YZaa),(Nai,Ci,YZab),(Nai,Ci,YZac),(Nai,Ci,YZad),(Nai,Ci,YZae),(Nai,Ci,YZaf),(Nai,Ci,YZ ag),(Nai,Cj,YZa),(Nai,Cj,YZb),(Nai,Cj,YZc),(Nai,Cj,YZcl),(Nai,Cj,YZe),(Nai,Cj,YZf),(Nai, Cj,YZg),(Nai,Cj,YZh),(Nai,Cj,YZi),(Nai,Cj,YZj),(Nai,Cj,YZk),(Nai,Cj,YZl),(Nai,Cj,YZm),( Nai,Cj,YZn),(Nai,Cj,YZo),(Nai,Cj,YZp),(Nai,Cj,YZq),(Nai,Cj,YZr),(Nai,Cj,YZs),(Nai,Cj,Y Zt),(Nai,Cj,YZu),(Nai,Cj,YZy),(Nai,Cj,YZw),(Nai,Cj,YZx),(Nai,Cj,YZy),(Nai,Cj,YZz),(Nai, Cj,YZaa),(Nai,Cj,YZab),(Nai,Cj,YZac),(Nai,Cj,YZad),(Nai,Cj,YZae),(Nai,Cj,YZaf),(Nai,Cj, YZag),(Naj,Ca,YZa),(Naj,Ca,YZb),(Naj,Ca,YZc),(Naj,Ca,YZd),(Naj,Ca,YZe),(Naj,Ca,YZf), (Naj,Ca,YZg),(Naj,Ca,YZh),(Naj,Ca,YZi),(Naj,Ca,YZj),(Naj,Ca,YZk),(Naj,Ca,YZl),(Naj,Ca, YZm),(Naj,Ca,YZn),(Naj,Ca,YZo),(Naj,Ca,YZp),(Naj,Ca,YZq),(Naj,Ca,YZr),(Naj,Ca,YZs),( Naj,Ca,YZf),(Naj,Ca,YZu),(Naj,Ca,YZy),(Naj,Ca,YZw),(Naj,Ca,YZx),(Naj,Ca,YZy),(Naj,C a,YZz),(Naj,Ca,YZaa),(Naj,Ca,YZab),(Naj,Ca,YZac),(Naj,Ca,YZad),(Naj,Ca,YZae),(Naj,Ca, YZaf),(Naj,Ca,YZag),(Naj,Cb,YZa),(Naj,Cb,YZb),(Naj,Cb,YZc),(Naj,Cb,YZd),(Naj,Cb,YZe) ,(Naj,Cb,YZf),(Naj,Cb,YZg),(Naj,Cb,YZh),(Naj,Cb,YZi),(Naj,Cb,YZj),(Naj,Cb,YZk),(Naj, Cb,YZl),(Naj,Cb,YZm),(Naj,Cb,YZn),(Naj,Cb,YZo),(Naj,Cb,YZp),(Naj,Cb,YZq),(Naj,Cb,Y Zr),(Naj,Cb,YZs),(Naj,Cb,YZt),(Naj,Cb,YZu),(Naj,Cb,YZv),(Naj,Cb,YZw),(Naj,Cb,YZx),( Naj,Cb,YZy),(Naj,Cb,YZz),(Naj,Cb,YZaa),(Naj,Cb,YZab),(Naj,Cb,YZac),(Naj,Cb,YZad),(N aj,Cb,YZae),(Naj,Cb,YZaf),(Naj,Cb,YZag),(Naj,Cc,YZa),(Naj,Cc,YZb),(Naj,Cc,YZc),(Naj, Cc,YZd),(Naj,Cc,YZe),(Naj,Cc,YZf),(Naj,Cc,YZg),(Naj,Cc,YZh),(Naj,Cc,YZi),(Naj,Cc,YZj) ,(Naj,Cc,YZk),(Naj,Cc,YZl),(Naj,Cc,YZm),(Naj,Cc,YZn),(Naj,Cc,YZo),(Naj,Cc,YZp),(Naj, Cc,YZq),(Naj,Cc,YZr),(Naj,Cc,YZs),(Naj,Cc,YZt),(Naj,Cc,YZu),(Naj,Cc,YZy),(Naj,Cc,YZ w),(Naj,Cc,YZx),(Naj,Cc,YZy),(Naj,Cc,YZz),(Naj,Cc,YZaa),(Naj,Cc,YZab),(Naj,Cc,YZac), (Naj,Cc,YZad),(Naj,Cc,YZae),(Naj,Cc,YZaf),(Naj,Cc,YZag),(Naj,Cd,YZa),(Naj,Cd,YZb),(N aj,Cd,YZc),(Naj,Cd,YZd),(Naj,Cd,YZe),(Naj,Cd,YZf),(Naj,Cd,YZg),(Naj,Cd,YZh),(Naj,Cd, YZi),(Naj,Cd,YZj),(Naj,Cd,YZk),(Naj,Cd,YZl),(Naj,Cd,YZm),(Naj,Cd,YZn),(Naj,Cd,YZo),( Naj,Cd,YZp),(Naj,Cd,YZq),(Naj,Cd,YZr),(Naj,Cd,YZs),(Naj,Cd,YZt),(Naj,Cd,YZu),(Naj,Cd ,YZNv),(Naj,Cd,YZw),(Naj,Cd,YZx),(Naj,Cd,YZy),(Naj,Cd,YZz),(Naj,Cd,YZaa),(Naj,Cd,YZ ab),(Naj,Cd,YZac),(Naj,Cd,YZad),(Naj,Cd,YZae),(Naj,Cd,YZaf),(Naj,Cd,YZag),(Naj,Ce,YZ a),(Naj,Ce,YZb),(Naj,Ce,YZc),(Naj,Ce,YZd),(Naj,Ce,YZe),(Naj,Ce,YZf),(Naj,Ce,YZg),(Naj, Ce,YZh),(Naj,Ce,YZi),(Naj,Ce,YZj),(Naj,Ce,YZk),(Naj,Ce,YZl),(Naj,Ce,YZm),(Naj,Ce,YZ n),(Naj,Ce,YZo),(Naj,Ce,YZp),(Naj,Ce,YZq),(Naj,Ce,YZr),(Naj,Ce,YZs),(Naj,Ce,YZt),(Naj, Ce,YZu),(Naj,Ce,YZy),(Naj,Ce,YZw),(Naj,Ce,YZx),(Naj,Ce,YZy),(Naj,Ce,YZz),(Naj,Ce,Y Zaa),(Naj,Ce,YZab),(Naj,Ce,YZac),(Naj,Ce,YZad),(Naj,Ce,YZae),(Naj,Ce,YZaf),(Naj,Ce,Y Zag),(Naj,Cf,YZa),(Naj,Cf,YZb),(Naj,Cf,YZc),(Naj,Cf,YZd),(Naj,Cf,YZe),(Naj,Cf,YZf),(Na j,Cf,YZg),(Naj,Cf,YZh),(Naj,Cf,YZi),(Naj,Cf,YZj),(Naj,Cf,YZk),(Naj,Cf,YZl),(Naj,Cf,YZm) ,(Naj,Cf,YZn),(Naj,Cf,YZo),(Naj,Cf,YZp),(Naj,Cf,YZq),(Naj,Cf,YZr),(Naj,Cf,YZs),(Naj,Cf ,YZt),(Naj,Cf,YZu),(Naj,Cf,YZy),(Naj,Cf,YZw),(Naj,Cf,YZx),(Naj,Cf,YZy),(Naj,Cf,YZz),( Naj,Cf,YZaa),(Naj,Cf,YZab),(Naj,Cf,YZac),(Naj,Cf,YZad),(Naj,Cf,YZae),(Naj,Cf,YZaf),(N aj,Cf,YZag),(Naj,Cg,YZa),(Naj,Cg,YZb),(Naj,Cg,YZc),(Naj,Cg,YZd),(Naj,Cg,YZe),(Naj,Cg ,YZf),(Naj,Cg,YZg),(Naj,Cg,YZh),(Naj,Cg,YZi),(Naj,Cg,YZj),(Naj,Cg,YZk),(Naj,Cg,YZl),( Naj,Cg,YZm),(Naj,Cg,YZn),(Naj,Cg,YZo),(Naj,Cg,YZp),(Naj,Cg,YZq),(Naj,Cg,YZr),(Naj, Cg,YZs),(Naj,Cg,YZt),(Naj,Cg,YZu),(Naj,Cg,YZAT),(Naj,Cg,YZw),(Naj,Cg,YZx),(Naj,Cg,Y Zy),(Naj,Cg,YZz),(Naj,Cg,YZaa),(Naj,Cg,YZab),(Naj,Cg,YZac),(Naj,Cg,YZad),(Naj,Cg,YZ ae),(Naj,Cg,YZaf),(Naj,Cg,YZag),(Naj,Ch,YZa),(Naj,Ch,YZb),(Naj,Ch,YZc),(Naj,Ch,YZd),( Naj,Ch,YZe),(Naj,Ch,YZf),(Naj,Ch,YZg),(Naj,Ch,YZh),(Naj,Ch,YZi),(Naj,Ch,YZj),(Naj,Ch, YZk),(Naj,Ch,YZl),(Naj,Ch,YZm),(Naj,Ch,YZn),(Naj,Ch,YZo),(Naj,Ch,YZp),(Naj,Ch,YZq) ,(Naj,Ch,YZr),(Naj,Ch,YZs),(Naj,Ch,YZt),(Naj,Ch,YZu),(Naj,Ch,YZy),(Naj,Ch,YZw),(Naj, Ch,YZx),(Naj,Ch,YZy),(Naj,Ch,YZz),(Naj,Ch,YZaa),(Naj,Ch,YZab),(Naj,Ch,YZac),(Naj,Ch ,YZad),(Naj,Ch,YZae),(Naj,Ch,YZaf),(Naj,Ch,YZag),(Naj,Ci,YZa),(Naj,Ci,YZb),(Naj,Ci,YZ c),(Naj,Ci,YZd),(Naj,Ci,YZe),(Naj,Ci,YZf),(Naj,Ci,YZg),(Naj,Ci,YZh),(Naj,Ci,YZi),(Naj,Ci ,YZj),(Naj,Ci,YZk),(Naj,Ci,YZl),(Naj,Ci,YZm),(Naj,Ci,YZn),(Naj,Ci,YZo),(Naj,Ci,YZp),(N aj,Ci,YZq),(Naj,Ci,YZr),(Naj,Ci,YZs),(Naj,Ci,YZt),(Naj,Ci,YZu),(Naj,Ci,YZy),(Naj,Ci,YZw) ,(Naj,Ci,YZx),(Naj,Ci,YZy),(Naj,Ci,YZz),(Naj,Ci,YZaa),(Naj,Ci,YZab),(Naj,Ci,YZac),(Naj, Ci,YZad),(Naj,Ci,YZae),(Naj,Ci,YZaf),(Naj,Ci,YZag),(Naj,Cj,YZa),(Naj,Cj,YZb),(Naj,Cj,Y Zc),(Naj,Cj,YZd),(Naj,Cj,YZe),(Naj,Cj,YZf),(Naj,Cj,YZg),(Naj,Cj,Ylh),(Naj,Cj,YZi),(Naj, Cj,YZj),(Naj,Cj,YZk),(Naj,Cj,YZl),(Naj,Cj,YZm),(Naj,Cj,YZn),(Naj,Cj,YZo),(Naj,Cj,YZp),( Naj,Cj,YZq),(Naj,Cj,YZr),(Naj,Cj,YZs),(Naj,Cj,YZt),(Naj,Cj,YZu),(Naj,Cj,YZv),(Naj,Cj,YZ w),(Naj,Cj,YZx),(Naj,Cj,YZy),(Naj,Cj,YZz),(Naj,Cj,YZaa),(Naj,Cj,YZab),(Naj,Cj,YZac),(N aj,Cj,YZad),(Naj,Cj,YZae),(Naj,Cj,YZaf),(Naj,Cj,YZag),(Nak,Ca,YZa),(Nak,Ca,YZb),(Nak, Ca,YZc),(Nak,Ca,YZd),(Nak,Ca,YZe),(Nak,Ca,YZf),(Nak,Ca,YZg),(Nak,Ca,YZh),(Nak,Ca, YZi),(Nak,Ca,YZj),(Nak,Ca,YZk),(Nak,Ca,YZl),(Nak,Ca,YZm),(Nak,Ca,YZn),(Nak,Ca,YZo) ,(Nak,Ca,YZp),(Nak,Ca,YZq),(Nak,Ca,YZr),(Nak,Ca,YZs),(Nak,Ca,YZt),(Nak,Ca,YZu),(N ak,Ca,YZy),(Nak,Ca,YZw),(Nak,Ca,YZx),(Nak,Ca,YZy),(Nak,Ca,YZz),(Nak,Ca,YZaa),(Na k,Ca,YZab),(Nak,Ca,YZac),(Nak,Ca,YZad),(Nak,Ca,YZae),(Nalc,Ca,YZaf),(Nak,Ca,YZag),( Nak,Cb,YZa),(Nak,Cb,YZb),(Nak,Cb,YZc),(Nak,Cb,YZd),(Nak,Cb,YZe),(Nak,Cb,YZf),(Na k,Cb,YZg),(Nak,Cb,YZh),(Nak,Cb,YZi),(Nak,Cb,YZj),(Nak,Cb,YZk),(Nak,Cb,YZl),(Nak,C b,YZm),(Nak,Cb,YZn),(Nak,Cb,YZo),(Nak,Cb,YZp),(Nak,Cb,YZq),(Nak,Cb,YZr),(Nak,Cb, YZs),(Nak,Cb,YZf),(Nak,Cb,YZu),(Nak,Cb,YZv),(Nak,Cb,YZw),(Nak,Cb,YZx),(Nak,Cb,Y Zy),(Nak,Cb,YZz),(Nak,Cb,YZaa),(Nak,Cb,YZab),(Nak,Cb,YZac),(Nak,Cb,YZad),(Nak,Cb, YZae),(Nak,Cb,YZaf),(Nak,Cb,YZag),(Nak,Cc,YZa),(Nak,Cc,YZb),(Nak,Cc,YZc),(Nak,Cc, YZd),(Nak,Cc,YZe),(Nak,Cc,YZf),(Nak,Cc,YZg),(Nalc,Cc,YZh),(Nak,Cc,YZi),(Nak,Cc,YZj) ,(Nak,Cc,YZk),(Nak,Cc,YZl),(Nak,Cc,YZm),(Nak,Cc,YZn),(Nak,Cc,YZo),(Nak,Cc,YZp),( Nak,Cc,YZq),(Nak,Cc,YZr),(Nak,Cc,YZs),(Nak,Cc,YZt),(Nak,Cc,YZu),(Nak,Cc,YZv),(Nak, Cc,YZw),(Nak,Cc,YZx),(Nak,Cc,YZy),(Nak,Cc,YZz),(Nak,Cc,YZaa),(Nak,Cc,YZab),(Nak, Cc,YZac),(Nak,Cc,YZad),(Nak,Cc,YZae),(Nak,Cc,YZaf),(Nak,Cc,YZag),(Nak,Cd,YZa),(Na k,Cd,YZb),(Nak,Cd,YZc),(Nalc,Cd,YZd),(Nak,Cd,YZe),(Nak,Cd,YZf),(Nak,Cd,YZg),(Nak, Cd,YZh),(Nak,Cd,YZi),(Nak,Cd,YZj),(Nak,Cd,YZk),(Nak,Cd,YZl),(Nak,Cd,YZm),(Nak,Cd, YZn),(Nak,Cd,YZo),(Nak,Cd,YZp),(Nak,Cd,YZq),(Nak,Cd,YZr),(Nak,Cd,YZs),(Nak,Cd,YZ t),(Nak,Cd,YZu),(Nak,Cd,YZv),(Nak,Cd,YZw),(Nak,Cd,YZx),(Nak,Cd,YZy),(Nak,Cd,YZz), (Nak,Cd,YZaa),(Nak,Cd,YZab),(Nak,Cd,YZac),(Nak,Cd,YZad),(Nak,Cd,YZae),(Nak,Cd,YZ af),(Nak,Cd,YZag),(Nak,Ce,YZa),(Nak,Ce,YZb),(Nak,Ce,YZc),(Nak,Ce,YZd),(Nak,Ce,YZe) ,(Nak,Ce,YZf),(Nak,Ce,YZg),(Nak,Ce,YZh),(Nak,Ce,YZi),(Nak,Ce,YZj),(Nak,Ce,YZk),(Na k,Ce,YZl),(Nak,Ce,YZm),(Nak,Ce,YZn),(Nak,Ce,YZo),(Nak,Ce,YZp),(Nak,Ce,YZq),(Nak, Ce,YZr),(Nak,Ce,YZs),(Nak,Ce,YZf),(Nak,Ce,YZu),(Nak,Ce,YZv),(Nak,Ce,YZw),(Nak,Ce, YZx),(Nak,Ce,YZy),(Nak,Ce,YZz),(Nak,Ce,YZaa),(Nak,Ce,YZab),(Nak,Ce,YZac),(Nak,Ce, YZad),(Nak,Ce,YZae),(Nak,Ce,YZaf),(Nak,Ce,YZag),(Nak,Cf,YZa),(Nak,Cf,YZb),(Nak,Cf, YZc),(Nak,Cf,YZd),(Nak,Cf,YZe),(Nak,Cf,YZf),(Nak,Cf,YZg),(Nak,Cf,YZh),(Nak,Cf,YZi), (Nak,Cf,YZj),(Nak,Cf,YZk),(Nak,Cf,YZl),(Nak,Cf,YZm),(Nak,Cf,YZn),(Nak,Cf,YZo),(Nak, Cf,YZp),(Nak,Cf,YZq),(Nak,Cf,YZr),(Nak,Cf,YZs),(Nak,Cf,YZt),(Nak,Cf,YZu),(Nak,Cf,YZ v),(Nak,Cf,YZw),(Nak,Cf,YZx),(Nak,Cf,YZy),(Nak,Cf,YZz),(Nak,Cf,YZaa),(Nak,Cf,YZab), (Nak,Cf,YZac),(Nak,Cf,YZad),(Nak,Cf,YZae),(Nak,Cf,YZaf),(Nak,Cf,YZag),(Nak,Cg,YZa), (Nak,Cg,YZb),(Nak,Cg,YZc),(Nak,Cg,YZd),(Nak,Cg,YZe),(Nak,Cg,YZf),(Nak,Cg,YZg),(N ak,Cg,YZh),(Nak,Cg,YZi),(Nak,Cg,YZj),(Nak,Cg,YZk),(Nak,Cg,YZl),(Nak,Cg,YZm),(Nak, Cg,YZn),(Nak,Cg,YZo),(Nak,Cg,YZp),(Nak,Cg,YZq),(Nak,Cg,YZr),(Nak,Cg,YZs),(Nak,Cg ,YZt),(Nak,Cg,YZu),(Nak,Cg,YZv),(Nak,Cg,YZw),(Nak,Cg,YZx),(Nak,Cg,YZy),(Nak,Cg,Y Zz),(Nak,Cg,YZaa),(Nak,Cg,YZab),(Nak,Cg,YZac),(Nak,Cg,YZad),(Nak,Cg,YZae),(Nak,Cg ,YZaf),(Nak,Cg,YZag),(Nak,Ch,YZa),(Nak,Ch,YZb),(Nak,Ch,YZc),(Nak,Ch,Yal),(Nak,Ch, YZe),(Nak,Ch,YZf),(Nak,Ch,YZg),(Nak,Ch,YZh),(Nak,Ch,YZi),(Nak,Ch,YZj),(Nak,Ch,YZ k),(Nak,Ch,YZl),(Nak,Ch,YZm),(Nak,Ch,YZn),(Nak,Ch,YZo),(Nak,Ch,YZp),(Nak,Ch,YZq) ,(Nak,Ch,YZr),(Nak,Ch,YZs),(Nak,Ch,YZt),(Nak,Ch,YZu),(Nak,Ch,YZv),(Nak,Ch,YZw),(N ak,Ch,YZx),(Nak,Ch,YZy),(Nak,Ch,YZz),(Nak,Ch,YZaa),(Nak,Ch,YZab),(Nak,Ch,YZac),( Nak,Ch,YZad),(Nak,Ch,YZae),(Nak,Ch,YZaf),(Nak,Ch,YZag),(Nak,Ci,YZa),(Nak,Ci,YZb),( Nak,Ci,YZc),(Nak,Ci,YZd),(Nak,Ci,YZe),(Nak,Ci,YZf),(Nak,Ci,YZg),(Nak,Ci,YZh),(Nak,C i,YZi),(Nak,Ci,YZj),(Nak,Ci,YZk),(Nak,Ci,YZl),(Nak,Ci,YZm),(Nak,Ci,YZn),(Nak,Ci,YZo) ,(Nak,Ci,YZp),(Nak,Ci,YZq),(Nak,Ci,YZr),(Nak,Ci,YZs),(Nak,Ci,YZt),(Nak,Ci,YZu),(Nak, Ci,YZy),(Nak,Ci,YZw),(Nak,Ci,YZx),(Nak,Ci,YZy),(Nak,Ci,YZz),(Nak,Ci,YZaa),(Nak,Ci, YZab),(Nak,Ci,YZac),(Nak,Ci,YZad),(Nak,Ci,YZae),(Nak,Ci,YZaf),(Nak,Ci,YZag),(Nak,Cj, YZa),(Nak,Cj,YZb),(Nak,Cj,YZc),(Nak,Cj,YZd),(Nak,Cj,YZe),(Nak,Cj,YZf),(Nak,Cj,YZg),( Nak,Cj,YZh),(Nak,Cj,YZi),(Nak,Cj,YZj),(Nak,Cj,YZk),(Nak,Cj,YZl),(Nak,Cj,YZm),(Nak,C j,YZn),(Nak,Cj,YZo),(Nak,Cj,YZp),(Nak,Cj,YZq),(Nak,Cj,YZr),(Nak,Cj,YZs),(Nak,Cj,YZt), (Nak,Cj,YZu),(Nak,Cj,YZy),(Nak,Cj,YZw),(Nak,Cj,YZx),(Nak,Cj,YZy),(Nak,Cj,YZz),(Nak, Cj,YZaa),(Nak,Cj,YZab),(Nak,Cj,YZac),(Nak,Cj,YZad),(Nak,Cj,YZae),(Nak,Cj,YZaf),(Nak, Cj,YZag),(Nal,Ca,YZa),(Nal,Ca,YZb),(Nal,Ca,YZc),(Nal,Ca,YZd),(Nal,Ca,YZe),(Nal,Ca,Y Zf),(Nal,Ca,YZg),(Nal,Ca,YZh),(Nal,Ca,YZi),(Nal,Ca,YZj),(Nal,Ca,YZk),(Nal,Ca,YZl),(Nal ,Ca,YZm),(Nal,Ca,YZn),(Nal,Ca,YZo),(Nal,Ca,YZp),(Nal,Ca,YZq),(Nal,Ca,YZr),(Nal,Ca,Y Zs),(Nal,Ca,YZf),(Nal,Ca,YZu),(Nal,Ca,YZy),(Nal,Ca,YZw),(Nal,Ca,YZx),(Nal,Ca,YZy),(N al,Ca,YZz),(Nal,Ca,YZaa),(Nal,Ca,YZab),(Nal,Ca,YZac),(Nal,Ca,YZad),(Nal,Ca,YZae),(Nal ,Ca,YZaf),(Nal,Ca,YZag),(Nal,Cb,YZa),(Nal,Cb,YZb),(Nal,Cb,YZc),(Nal,Cb,YZd),(Nal,Cb, YZe),(Nal,Cb,YZf),(Nal,Cb,YZg),(Nal,Cb,YZh),(Nal,Cb,YZi),(Nal,Cb,YZj),(Nal,Cb,YZk),( Nal,Cb,YZl),(Nal,Cb,YZm),(Nal,Cb,YZn),(Nal,Cb,YZo),(Nal,Cb,YZp),(Nal,Cb,YZq),(Nal,C b,YZr),(Nal,Cb,YZs),(Nal,Cb,YZt),(Nal,Cb,YZu),(Nal,Cb,YZy),(Nal,Cb,YZw),(Nal,Cb,YZx) ,(Nal,Cb,YZy),(Nal,Cb,YZz),(Nal,Cb,YZaa),(Nal,Cb,YZab),(Nal,Cb,YZac),(Nal,Cb,YZad), (Nal,Cb,YZae),(Nal,Cb,YZaf),(Nal,Cb,YZag),(Nal,Cc,YZa),(Nal,Cc,YZb),(Nal,Cc,YZc),(Na l1,Cc,YZd),(Nal,Cc,YZe),(Nal,Cc,YZf),(Nal,Cc,YZg),(Nal,Cc,YZh),(Nal,Cc,YZi),(Nal,Cc,YZ j),(Nal,Cc,YZk),(Nal,Cc,YZl),(Nal,Cc,YZm),(Nal,Cc,YZn),(Nal,Cc,YZo),(Nal,Cc,YZp),(Nal ,Cc,YZq),(Nal,Cc,YZr),(Nal,Cc,YZs),(Nal,Cc,YZt),(Nal,Cc,YZu),(Nal,Cc,YZy),(Nal,Cc,YZ w),(Nal,Cc,YZx),(Nal,Cc,YZy),(Nal,Cc,YZz),(Nal,Cc,YZaa),(Nal,Cc,YZab),(Nal,Cc,YZac), (Nal,Cc,YZad),(Nal,Cc,YZae),(Nal,Cc,YZaf),(Nal,Cc,YZag),(Nal,Cd,YZa),(Nal,Cd,YZb),(N al,Cd,YZc),(Nal,Cd,YZcl),(Nal,Cd,YZe),(Nal,Cd,YZf),(Nal,Cd,YZg),(Nal,Cd,Ya),(Nal,Cd, YZi),(Nal,Cd,YZj),(Nal,Cd,YZk),(Nal,Cd,YZl),(Nal,Cd,YZm),(Nal,Cd,YZn),(Nal,Cd,YZo),( Nal,Cd,YZp),(Nal,Cd,YZq),(Nal,Cd,YZr),(Nal,Cd,YZs),(Nal,Cd,YZt),(Nal,Cd,YZu),(Nal,Cd ,YZy),(Nal,Cd,YZw),(Nal,Cd,YZx),(Nal,Cd,YZy),(Nal,Cd,YZz),(Nal,Cd,YZaa),(Nal,Cd,YZ ab),(Nal,Cd,YZac),(Nal,Cd,YZad),(Nal,Cd,YZae),(Nal,Cd,YZaf),(Nal,Cd,YZag),(Nal,Ce,YZ a),(Nal,Ce,YZb),(Nal,Ce,YZc),(Nal,Ce,YZd),(Nal,Ce,YZe),(Nal,Ce,YZf),(Nal,Ce,YZg),(Nal, Ce,YZh),(Nal,Ce,YZi),(Nal,Ce,YZj),(Nal,Ce,YZk),(Nal,Ce,YZl),(Nal,Ce,YZm),(Nal,Ce,YZ n),(Nal,Ce,YZo),(Nal,Ce,YZp),(Nal,Ce,YZq),(Nal,Ce,YZr),(Nal,Ce,YZs),(Nal,Ce,YZt),(Nal, Ce,YZu),(Nal,Ce,Ylv),(Nal,Ce,YZw),(Nal,Ce,YZx),(Nal,Ce,YZy),(Nal,Ce,YZz),(Nal,Ce,Y Zaa),(Nal,Ce,YZab),(Nal,Ce,YZac),(Nal,Ce,YZad),(Nal,Ce,YZae),(Nal,Ce,YZaf),(Nal,Ce,Y Zag),(Nal,Cf,YZa),(Nal,Cf,YZb),(Nal,Cf,YZc),(Nal,Cf,YZd),(Nal,Cf,YZe),(Nal,Cf,YZf),(Na l,Cf,YZg),(Nal,Cf,YZh),(Nal,Cf,YZi),(Nal,Cf,YZj),(Nal,Cf,YZk),(Nal,Cf,YZl),(Nal,Cf,YZm) ,(Nal,Cf,YZn),(Nal,Cf,YZo),(Nal,Cf,YZp),(Nal,Cf,YZq),(Nal,Cf,YZr),(Nal,Cf,YZs),(Nal,Cf ,YZt),(Nal,Cf,YZu),(Nal,Cf,YZy),(Nal,Cf,YZw),(Nal,Cf,YZx),(Nal,Cf,YZy),(Nal,Cf,YZz),( Nal,Cf,YZaa),(Nal,Cf,YZab),(Nal,Cf,YZac),(Nal,Cf,YZad),(Nal,Cf,YZae),(Nal,Cf,YZaf),(N al,Cf,YZag),(Nal,Cg,YZa),(Nal,Cg,YZb),(Nal,Cg,YZc),(Nal,Cg,YZd),(Nal,Cg,YZe),(Nal,Cg ,YZf),(Nal,Cg,YZg),(Nal,Cg,YZh),(Nal,Cg,YZi),(Nal,Cg,YZj),(Nal,Cg,YZk),(Nal,Cg,YZl),( Nal,Cg,YZm),(Nal,Cg,YZn),(Nal,Cg,YZo),(Nal,Cg,YZp),(Nal,Cg,YZq),(Nal,Cg,YZr),(Nal, Cg,YZs),(Nal,Cg,YZt),(Nal,Cg,YZu),(Nal,Cg,YZy),(Nal,Cg,YZw),(Nal,Cg,YZx),(Nal,Cg,Y Zy),(Nal,Cg,YZz),(Nal,Cg,YZaa),(Nal,Cg,YZab),(Nal,Cg,YZac),(Nal,Cg,YZad),(Nal,Cg,YZ ae),(Nal,Cg,YZaf),(Nal,Cg,YZag),(Nal,Ch,YZa),(Nal,Ch,YZb),(Nal,Ch,YZc),(Nal,Ch,YZd),( Nal,Ch,YZe),(Nal,Ch,Ya),(Nal,Ch,YZg),(Nal,Ch,Ya),(Nal,Ch,YZi),(Nal,Ch,YZj),(Nal,Ch, YZk),(Nal,Ch,YZl),(Nal,Ch,YZm),(Nal,Ch,YZn),(Nal,Ch,YZo),(Nal,Ch,YZp),(Nal,Ch,YZq) ,(Nal,Ch,YZr),(Nal,Ch,YZs),(Nal,Ch,YZf),(Nal,Ch,YZu),(Nal,Ch,YZy),(Nal,Ch,YZw),(Nal, Ch,YZx),(Nal,Ch,YZy),(Nal,Ch,YZz),(Nal,Ch,YZaa),(Nal,Ch,YZab),(Nal,Ch,YZac),(Nal,Ch ,YZad),(Nal,Ch,YZae),(Nal,Ch,YZaf),(Nal,Ch,YZag),(Nal,Ci,YZa),(Nal,Ci,YZb),(Nal,Ci,YZ c),(Nal,Ci,YZd),(Nal,Ci,YZe),(Nal,Ci,YZf),(Nal,Ci,YZg),(Nal,Ci,YZh),(Nal,Ci,YZi),(Nal,Ci ,YZj),(Nal,Ci,YZk),(Nal,Ci,YZl),(Nal,Ci,YZm),(Nal,Ci,YZn),(Nal,Ci,YZo),(Nal,Ci,YZp),(N al,Ci,YZq),(Nal,Ci,YZr),(Nal,Ci,YZs),(Nal,Ci,YZt),(Nal,YZu),(Nal,Ci,YZv),(Nal,Ci,YZw ),(Nal,Ci,YZx),(Nal,Ci,YZy),(Nal,Ci,YZz),(Nal,Ci,YZaa),(Nal,Ci,YZab),(Nal,Ci,YZac),(Nal, Ci,YZad),(Nal,Ci,YZae),(Nal,Ci,YZaf),(Nal,Ci,YZag),(Nal,Cj,YZa),(Nal,Cj,YZb),(Nal,Cj,Y Zc),(Nal,Cj,YZd),(Nal,Cj,YZe),(Nal,Cj,YZf),(Nal,Cj,YZg),(Nal,Cj,YZh),(Nal,Cj,YZi),(Nal, Cj,YZj),(Nal,Cj,YZk),(Nal,Cj,YZl),(Nal,q,YZm),(Nal,Cj,YZn),(Nal,Cj,YZo),(Nal,Cj,YZp),( Nal,Cj,YZq),(Nal,Cj,YZr),(Nal,Cj,YZs),(Nal,Cj,YZt),(Nal,Cj,YZu),(Nal,Cj,YZy),(Nal,Cj,YZ w),(Nal,Cj,YZx),(Nal,Cj,YZy),(Nal,Cj,YZz),(Nal,Cj,YZaa),(Nal,Cj,YZab),(Nal,Cj,YZac),(N al,Cj,YZad),(Nal,Cj,YZae),(Nal,Cj,YZaf),(Nal,Cj,YZag),(Nam,Ca,YZa),(Nam,Ca,YZb),(Na m,Ca,YZc),(Nam,Ca,YZd),(Nam,Ca,YZe),(Nam,Ca,YZf),(Nam,Ca,YZg),(Nam,Ca,YZh),(Na m,Ca,YZi),(Nam,Ca,YZj),(Nam,Ca,YZk),(Nam,Ca,YZl),(Nam,Ca,YZm),(Nam,Ca,YZn),(Na m,Ca,YZo),(Nam,Ca,YZp),(Nam,Ca,YZq),(Nam,Ca,YZr),(Nam,Ca,YZs),(Nam,Ca,YZt),(Na m,Ca,YZu),(Nam,Ca,YZy),(Nam,Ca,YZw),(Nam,Ca,YZx),(Nam,Ca,YZy),(Nam,Ca,YZz),(N am,Ca,YZaa),(Nam,Ca,YZab),(Nam,Ca,YZac),(Nam,Ca,YZad),(Nam,Ca,YZae),(Nam,Ca,Y Zaf),(Nam,Ca,YZag),(Nam,Cb,YZa),(Nam,Cb,YZb),(Nam,Cb,YZc),(Nam,Cb,YZd),(Nam,C b,YZe),(Nam,Cb,YZf),(Nam,Cb,YZg),(Nam,Cb,YZh),(Nam,Cb,YZi),(Nam,Cb,YZj),(Nam,C b,YZk),(Nam,Cb,YZl),(Nam,Cb,YZm),(Nam,Cb,YZn),(Nam,Cb,YZo),(Nam,Cb,YZp),(Nam, Cb,YZq),(Nam,Cb,YZr),(Nam,Cb,YZs),(Nam,Cb,YZt),(Nam,Cb,YZu),(Nam,Cb,YZy),(Nam ,Cb,YZw),(Nam,Cb,YZx),(Nam,Cb,YZy),(Nam,Cb,YZz),(Nam,Cb,YZaa),(Nam,Cb,YZab),( Nam,Cb,YZac),(Nam,Cb,YZad),(Nam,Cb,YZae),(Nam,Cb,YZaf),(Nam,Cb,YZag),(Nam,Cc, YZa),(Nam,Cc,YZb),(Nam,Cc,YZc),(Nam,Cc,YZd),(Nam,Cc,YZe),(Nam,Cc,YZf),(Nam,Cc, YZg),(Nam,Cc,YZh),(Nam,Cc,YZi),(Nam,Cc,YZj),(Nam,Cc,YZk),(Nam,Cc,YZl),(Nam,Cc, YZm),(Nam,Cc,YZn),(Nam,Cc,YZo),(Nam,Cc,YZp),(Nam,Cc,YZq),(Nam,Cc,YZr),(Nam,C c,YZs),(Nam,Cc,YZt),(Nam,Cc,YZu),(Nam,Cc,YZy),(Nam,Cc,YZw),(Nam,Cc,YZx),(Nam, Cc,YZy),(Nam,Cc,YZz),(Nam,Cc,YZaa),(Nam,Cc,YZab),(Nam,Cc,YZac),(Nam,Cc,YZad),( Nam,Cc,YZae),(Nam,Cc,YZaf),(Nam,Cc,YZag),(Nam,Cd,YZa),(Nam,Cd,YZb),(Nam,Cd,Y Zc),(Nam,Cd,YZd),(Nam,Cd,YZe),(Nam,Cd,YZf),(Nam,Cd,YZg),(Nam,Cd,YZh),(Nam,Cd, YZi),(Nam,Cd,YZj),(Nam,Cd,YZk),(Nam,Cd,YZl),(Nam,Cd,YZm),(Nam,Cd,YZn),(Nam,Cd ,YZo),(Nam,Cd,YZp),(Nam,Cd,YZq),(Nam,Cd,YZr),(Nam,Cd,YZs),(Nam,Cd,YZt),(Nam,C d,YZu),(Nam,Cd,YZy),(Nam,Cd,YZw),(Nam,Cd,YZx),(Nam,Cd,YZy),(Nam,Cd,YZz),(Nam ,Cd,YZaa),(Nam,Cd,YZab),(Nam,Cd,YZac),(Nam,Cd,YZad),(Nam,Cd,YZae),(Nam,Cd,YZa f),(Nam,Cd,YZag),(Nam,Ce,YZa),(Nam,Ce,YZb),(Nam,Ce,YZc),(Nam,Ce,YZd),(Nam,Ce,Y Ze),(Nam,Ce,YZf),(Nam,Ce,YZg),(Nam,Ce,YZh),(Nam,Ce,YZi),(Nam,Ce,YZj),(Nam,Ce,Y Zk),(Nam,Ce,YZl),(Nam,Ce,YZm),(Nam,Ce,YZn),(Nam,Ce,YZo),(Nam,Ce,YZp),(Nam,Ce, YZq),(Nam,Ce,YZr),(Nam,Ce,YZs),(Nam,Ce,YZt),(Nam,Ce,YZu),(Nam,Ce,YZy),(Nam,Ce, YZw),(Nam,Ce,YZx),(Nam,Ce,YZy),(Nam,Ce,YZz),(Nam,Ce,YZaa),(Nam,Ce,YZab),(Nam, Ce,YZac),(Nam,Ce,YZad),(Nam,Ce,YZae),(Nam,Ce,YZaf),(Nam,Ce,YZag),(Nam,Cf,YZa),( Nam,Cf,YZb),(Nam,Cf,YZc),(Nam,Cf,YZd),(Nam,Cf,YZe),(Nam,Cf,YZf),(Nam,Cf,YZg),(N am,Cf,YZh),(Nam,Cf,YZi),(Nam,Cf,YZj),(Nam,Cf,YZk),(Nam,Cf,YZl),(Nam,Cf,YZm),(Na m,Cf,YZn),(Nam,Cf,YZo),(Nam,Cf,YZp),(Nam,Cf,YZq),(Nam,Cf,YZr),(Nam,Cf,YZs),(Na m,Cf,YZt),(Nam,Cf,YZu),(Nam,Cf,YZy),(Nam,Cf,YZw),(Nam,Cf,YZx),(Nam,Cf,YZy),(Na m,Cf,YZz),(Nam,Cf,YZaa),(Nam,Cf,YZab),(Nam,Cf,YZac),(Nam,Cf,YZad),(Nam,Cf,YZae) ,(Nam,Cf,YZaf),(Nam,Cf,YZag),(Nam,Cg,YZa),(Nam,Cg,YZb),(Nam,Cg,YZc),(Nam,Cg,YZ d),(Nam,Cg,YZe),(Nam,Cg,YZf),(Nam,Cg,YZg),(Nam,Cg,YZh),(Nam,Cg,YZi),(Nam,Cg,Y Zj),(Nam,Cg,YZk),(Nam,Cg,YZl),(Nam,Cg,YZm),(Nam,Cg,YZn),(Nam,Cg,YZo),(Nam,Cg, YZp),(Nam,Cg,YZq),(Nam,Cg,YZr),(Nam,Cg,YZs),(Nam,Cg,YZt),(Nam,Cg,YZu),(Nam,Cg ,YZv),(Nam,Cg,YZw),(Nam,Cg,YZx),(Nam,Cg,YZy),(Nam,Cg,YZz),(Nam,Cg,YZaa),(Nam, Cg,YZab),(Nam,Cg,YZac),(Nam,Cg,YZad),(Nam,Cg,YZae),(Nam,Cg,YZaf),(Nam,Cg,YZag) ,(Nam,Ch,YZa),(Nam,Ch,YZb),(Nam,Ch,YZc),(Nam,Ch,YZd),(Nam,Ch,YZe),(Nam,Ch,YZ f),(Nam,Ch,YZg),(Nam,Ch,YZh),(Nam,Ch,YZi),(Nam,Ch,YZj),(Nam,Ch,YZk),(Nam,Ch,YZ l),(Nam,Ch,YZm),(Nam,Ch,YZn),(Nam,Ch,YZo),(Nam,Ch,YZp),(Nam,Ch,YZq),(Nam,Ch,Y Zr),(Nam,Ch,YZs),(Nam,Ch,YZt),(Nam,Ch,YZu),(Nam,Ch,YZy),(Nam,Ch,YZw),(Nam,Ch, YZx),(Nam,Ch,YZy),(Nam,Ch,YZz),(Nam,Ch,YZaa),(Nam,Ch,YZab),(Nam,Ch,YZac),(Na m,Ch,YZad),(Nam,Ch,YZae),(Nam,Ch,YZaf),(Nam,Ch,YZag),(Nam,Ci,YZa),(Nam,Ci,YZb) ,(Nam,Ci,YZc),(Nam,Ci,YZd),(Nam,Ci,YZe),(Nam,Ci,YZf),(Nam,Ci,YZg),(Nam,Ci,YZh),( Nam,Ci,YZi),(Nam,Ci,YZj),(Nam,Ci,YZk),(Nam,Ci,YZl),(Nam,Ci,YZm),(Nam,Ci,YZn),(Na m,Ci,YZo),(Nam,Ci,YZp),(Nam,Ci,YZq),(Nam,Ci,YZr),(Nam,Ci,YZs),(Nam,Ci,YZt),(Nam, Ci,YZu),(Nam,Ci,YZy),(Nam,Ci,YZw),(Nam,Ci,YZx),(Nam,Ci,YZy),(Nam,Ci,YZz),(Nam, Ci,YZaa),(Nam,Ci,YZab),(Nam,Ci,YZac),(Nam,Ci,YZad),(Nam,Ci,YZae),(Nam,Ci,YZaf),( Nam,Ci,YZag),(Nam,Cj,YZa),(Nam,Cj,YZb),(Nam,Cj,YZc),(Nam,Cj,YZd),(Nam,Cj,YZe),( Nam,Cj,YZf),(Nam,Cj,YZg),(Nam,Cj,YZh),(Nam,Cj,YZi),(Nam,Cj,YZj),(Nam,Cj,YZk),(Na m,Cj,YZl),(Nam,Cj,YZm),(Nam,Cj,YZn),(Nam,Cj,YZo),(Nam,Cj,YZp),(Nam,Cj,YZq),(Na m,Cj,YZr),(Nam,Cj,YZs),(Nam,Cj,YZt),(Nam,Cj,YZu),(Nam,Cj,YZy),(Nam,Cj,YZw),(Nam, Cj,YZx),(Nam,Cj,YZy),(Nam,Cj,YZz),(Nam,Cj,YZaa),(Nam,Cj,YZab),(Nam,Cj,YZac),(Na m,Cj,YZad),(Nam,Cj,YZae),(Nam,Cj,YZaf),(Nam,Cj,YZag),(Nan,Ca,YZa),(Nan,Ca,YZb),( Nan,Ca,YZc),(Nan,Ca,YZd),(Nan,Ca,YZe),(Nan,Ca,YZf),(Nan,Ca,YZg),(Nan,Ca,YZh),(Nan ,Ca,YZi),(Nan,Ca,YZj),(Nan,Ca,YZk),(Nan,Ca,YZl),(Nan,Ca,YZm),(Nan,Ca,YZn),(Nan,Ca, YZo),(Nan,Ca,YZp),(Nan,Ca,YZq),(Nan,Ca,YZr),(Nan,Ca,YZs),(Nan,Ca,YZt),(Nan,Ca,YZu) ,(Nan,Ca,YZy),(Nan,Ca,YZw),(Nan,Ca,YZx),(Nan,Ca,YZy),(Nan,Ca,YZz),(Nan,Ca,YZaa), (Nan,Ca,YZab),(Nan,Ca,YZac),(Nan,Ca,YZad),(Nan,Ca,YZae),(Nan,Ca,YZaf),(Nan,Ca,YZa g),(Nan,Cb,YZa),(Nan,Cb,YZb),(Nan,Cb,YZc),(Nan,Cb,YZd),(Nan,Cb,YZe),(Nan,Cb,YZf),( Nan,Cb,YZg),(Nan,Cb,YZh),(Nan,Cb,YZi),(Nan,Cb,YZj),(Nan,Cb,YZk),(Nan,Cb,YZl),(Nan ,Cb,YZm),(Nan,Cb,YZn),(Nan,Cb,YZo),(Nan,Cb,YZp),(Nan,Cb,YZq),(Nan,Cb,YZr),(Nan,C b,YZs),(Nan,Cb,YZt),(Nan,Cb,YZu),(Nan,Cb,YZv),(Nan,Cb,YZw),(Nan,Cb,YZx),(Nan,Cb, YZy),(Nan,Cb,YZz),(Nan,Cb,YZaa),(Nan,Cb,YZab),(Nan,Cb,YZac),(Nan,Cb,YZad),(Nan,C b,YZae),(Nan,Cb,YZaf),(Nan,Cb,YZag),(Nan,Cc,YZa),(Nan,Cc,YZb),(Nan,Cc,YZc),(Nan,C c,YZd),(Nan,Cc,YZe),(Nan,Cc,YZf),(Nan,Cc,YZg),(Nan,Cc,YZh),(Nan,Cc,YZi),(Nan,Cc,Y Zj),(Nan,Cc,YZk),(Nan,Cc,YZl),(Nan,Cc,YZm),(Nan,Cc,YZn),(Nan,Cc,YZo),(Nan,Cc,YZp) ,(Nan,Cc,YZq),(Nan,Cc,YZr),(Nan,Cc,YZs),(Nan,Cc,YZt),(Nan,Cc,YZu),(Nan,Cc,YZy),(Na n,Cc,YZw),(Nan,Cc,YZx),(Nan,Cc,YZy),(Nan,Cc,YZz),(Nan,Cc,YZaa),(Nan,Cc,YZab),(Na n,Cc,YZac),(Nan,Cc,YZad),(Nan,Cc,YZae),(Nan,Cc,YZaf),(Nan,Cc,YZag),(Nan,Cd,YZa),( Nan,Cd,YZb),(Nan,Cd,YZc),(Nan,Cd,YZd),(Nan,Cd,YZe),(Nan,Cd,YZf),(Nan,Cd,YZg),(Na n,Cd,YZh),(Nan,Cd,YZi),(Nan,Cd,YZj),(Nan,Cd,YZk),(Nan,Cd,YZl),(Nan,Cd,YZm),(Nan,C d,YZn),(Nan,Cd,YZo),(Nan,Cd,YZp),(Nan,Cd,YZq),(Nan,Cd,YZr),(Nan,Cd,YZs),(Nan,Cd, YZf),(Nan,Cd,YZu),(Nan,Cd,YZy),(Nan,Cd,YZw),(Nan,Cd,YZx),(Nan,Cd,YZy),(Nan,Cd,Y Zz),(Nan,Cd,YZaa),(Nan,Cd,YZab),(Nan,Cd,YZac),(Nan,Cd,YZad),(Nan,Cd,YZae),(Nan,Cd ,YZaf),(Nan,Cd,YZag),(Nan,Ce,YZa),(Nan,Ce,YZb),(Nan,Ce,YZc),(Nan,Ce,YZd),(Nan,Ce, YZe),(Nan,Ce,YZf),(Nan,Ce,YZg),(Nan,Ce,YZh),(Nan,Ce,YZi),(Nan,Ce,YZj),(Nan,Ce,YZk) ,(Nan,Ce,YZl),(Nan,Ce,YZm),(Nan,Ce,YZn),(Nan,Ce,YZo),(Nan,Ce,YZp),(Nan,Ce,YZq),( Nan,Ce,YZr),(Nan,Ce,YZs),(Nan,Ce,YZt),(Nan,Ce,YZu),(Nan,Ce,YZy),(Nan,Ce,YZw),(Nan ,Ce,YZx),(Nan,Ce,YZy),(Nan,Ce,YZz),(Nan,Ce,YZaa),(Nan,Ce,YZab),(Nan,Ce,YZac),(Nan, Ce,YZad),(Nan,Ce,YZae),(Nan,Ce,YZaf),(Nan,Ce,YZag),(Nan,Cf,YZa),(Nan,Cf,YZb),(Nan, Cf,YZc),(Nan,Cf,YZd),(Nan,Cf,YZe),(Nan,Cf,YZf),(Nan,Cf,YZg),(Nan,Cf,YZh),(Nan,Cf,Y Zi),(Nan,Cf,YZj),(Nan,Cf,YZk),(Nan,Cf,YZl),(Nan,Cf,YZm),(Nan,Cf,YZn),(Nan,Cf,YZo),( Nan,Cf,YZp),(Nan,Cf,YZq),(Nan,Cf,YZr),(Nan,Cf,YZs),(Nan,Cf,YZf),(Nan,Cf,YZu),(Nan,C CYZNT),(Nan,Cf,YZw),(Nan,Cf,YZx),(Nan,Cf,YZy),(Nan,Cf,YZz),(Nan,Cf,YZaa),(Nan,Cf,Y Zab),(Nan,Cf,YZac),(Nan,Cf,YZad),(Nan,Cf,YZae),(Nan,Cf,YZaf),(Nan,Cf,YZag),(Nan,Cg, YZa),(Nan,Cg,YZb),(Nan,Cg,YZc),(Nan,Cg,YZd),(Nan,Cg,YZe),(Nan,Cg,YZf),(Nan,Cg,YZ g),(Nan,Cg,YZh),(Nan,Cg,YZi),(Nan,Cg,YZj),(Nan,Cg,YZk),(Nan,Cg,YZl),(Nan,Cg,YZm),( Nan,Cg,YZn),(Nan,Cg,YZo),(Nan,Cg,YZp),(Nan,Cg,YZq),(Nan,Cg,YZr),(Nan,Cg,YZs),(Na n,Cg,YZt),(Nan,Cg,YZu),(Nan,Cg,YZy),(Nan,Cg,YZw),(Nan,Cg,YZx),(Nan,Cg,YZy),(Nan, Cg,YZz),(Nan,Cg,YZaa),(Nan,Cg,YZab),(Nan,Cg,YZac),(Nan,Cg,YZad),(Nan,Cg,YZae),(N an,Cg,YZaf),(Nan,Cg,YZag),(Nan,Ch,YZa),(Nan,Ch,YZb),(Nan,Ch,YZc),(Nan,Ch,YZd),(Na n,Ch,YZe),(Nan,Ch,YZf),(Nan,Ch,YZg),(Nan,Ch,YZh),(Nan,Ch,YZi),(Nan,Ch,YZj),(Nan,C h,YZk),(Nan,Ch,YZl),(Nan,Ch,YZm),(Nan,Ch,YZn),(Nan,Ch,YZo),(Nan,Ch,YZp),(Nan,Ch, YZq),(Nan,Ch,YZr),(Nan,Ch,YZs),(Nan,Ch,YZt),(Nan,Ch,YZu),(Nan,Ch,YZy),(Nan,Ch,YZ w),(Nan,Ch,YZx),(Nan,Ch,YZy),(Nan,Ch,YZz),(Nan,Ch,YZaa),(Nan,Ch,YZab),(Nan,Ch,YZ ac),(Nan,Ch,YZad),(Nan,Ch,YZae),(Nan,Ch,YZaf),(Nan,Ch,YZag),(Nan,Ci,YZa),(Nan,Ci,Y Zb),(Nan,Ci,YZc),(Nan,Ci,YZd),(Nan,Ci,YZe),(Nan,Ci,YZf),(Nan,Ci,YZg),(Nan,Ci,YZh),( Nan,Ci,YZi),(Nan,Ci,YZj),(Nan,Ci,YZk),(Nan,Ci,YZl),(Nan,Ci,YZm),(Nan,Ci,YZn),(Nan,C i,YZo),(Nan,Ci,YZp),(Nan,Ci,YZq),(Nan,Ci,YZr),(Nan,Ci,YZs),(Nan,Ci,YZt),(Nan,Ci,YZu), (Nan,Ci,YZy),(Nan,Ci,YZw),(Nan,Ci,YZx),(Nan,Ci,YZy),(Nan,Ci,YZz),(Nan,Ci,YZaa),(Na n,Ci,YZab),(Nan,Ci,YZac),(Nan,Ci,YZad),(Nan,Ci,YZae),(Nan,Ci,YZaf),(Nan,Ci,YZag),(Na n,Cj,YZa),(Nan,Cj,YZb),(Nan,Cj,YZc),(Nan,Cj,YZd),(Nan,Cj,YZe),(Nan,Cj,YZf),(Nan,Cj,Y Zg),(Nan,Cj,YZh),(Nan,Cj,YZi),(Nan,Cj,YZj),(Nan,Cj,YZk),(Nan,Cj,YZl),(Nan,Cj,YZm),(N an,Cj,YZn),(Nan,Cj,YZo),(Nan,Cj,YZp),(Nan,Cj,YZq),(Nan,Cj,YZr),(Nan,Cj,YZs),(Nan,Cj, YZt),(Nan,Cj,YZu),(Nan,Cj,YZy),(Nan,Cj,YZw),(Nan,Cj,YZx),(Nan,Cj,YZy),(Nan,Cj,YZz), (Nan,Cj,YZaa),(Nan,Cj,YZab),(Nan,Cj,YZac),(Nan,Cj,YZad),(Nan,Cj,YZae),(Nan,q,YZaf), (Nan,Cj,YZag),(Nao,Ca,YZa),(Nao,Ca,YZb),(Nao,Ca,YZc),(Nao,Ca,YZd),(Nao,Ca,YZe),(N ao,Ca,Ya),(Nao,Ca,YZg),(Nao,Ca,YZh),(Nao,Ca,YZi),(Nao,Ca,YZj),(Nao,Ca,YZlc),(Nao,C a,YZl),(Nao,Ca,YZm),(Nao,Ca,YZn),(Nao,Ca,YZo),(Nao,Ca,YZp),(Nao,Ca,YZq),(Nao,Ca, YZr),(Nao,Ca,YZs),(Nao,Ca,YZt),(Nao,Ca,YZu),(Nao,Ca,YZy),(Nao,Ca,YZw),(Nao,Ca,YZ x),(Nao,Ca,YZy),(Nao,Ca,YZz),(Nao,Ca,YZaa),(Nao,Ca,YZab),(Nao,Ca,YZac),(Nao,Ca,YZ ad),(Nao,Ca,YZae),(Nao,Ca,YZaf),(Nao,Ca,YZag),(Nao,Cb,YZa),(Nao,Cb,YZb),(Nao,Cb,Y Zc),(Nao,Cb,YZd),(Nao,Cb,YZe),(Nao,Cb,YZf),(Nao,Cb,YZg),(Nao,Cb,Ya),(Nao,Cb,YZi) ,(Nao,Cb,YZj),(Nao,Cb,YZk),(Nao,Cb,YZl),(Nao,Cb,YZm),(Nao,Cb,YZn),(Nao,Cb,YZo),( Nao,Cb,YZp),(Nao,Cb,YZq),(Nao,Cb,YZr),(Nao,Cb,YZs),(Nao,Cb,YZt),(Nao,Cb,YZu),(Na o,Cb,YZy),(Nao,Cb,YZw),(Nao,Cb,YZx),(Nao,Cb,YZy),(Nao,Cb,YZz),(Nao,Cb,YZaa),(Nao ,Cb,YZab),(Nao,Cb,YZac),(Nao,Cb,YZad),(Nao,Cb,YZae),(Nao,Cb,YZaf),(Nao,Cb,YZag),( Nao,Cc,YZa),(Nao,Cc,YZb),(Nao,Cc,YZc),(Nao,Cc,YZd),(Nao,Cc,YZe),(Nao,Cc,YZf),(Nao ,Cc,YZg),(Nao,Cc,YZh),(Nao,Cc,YZi),(Nao,Cc,YZj),(Nao,Cc,YZk),(Nao,Cc,YZl),(Nao,Cc, YZm),(Nao,Cc,YZn),(Nao,Cc,YZo),(Nao,Cc,YZp),(Nao,Cc,YZq),(Nao,Cc,YZr),(Nao,Cc,YZ s),(Nao,Cc,YZt),(Nao,Cc,YZu),(Nao,Cc,YZy),(Nao,Cc,YZw),(Nao,Cc,YZx),(Nao,Cc,YZy),( Nao,Cc,YZz),(Nao,Cc,YZaa),(Nao,Cc,YZab),(Nao,Cc,YZac),(Nao,Cc,YZad),(Nao,Cc,YZae) ,(Nao,Cc,YZaf),(Nao,Cc,YZag),(Nao,Cd,YZa),(Nao,Cd,YZb),(Nao,Cd,YZc),(Nao,Cd,YZd),( Nao,Cd,YZe),(Nao,Cd,YZf),(Nao,Cd,YZg),(Nao,Cd,YZh),(Nao,Cd,YZi),(Nao,Cd,YZj),(Nao ,Cd,YZk),(Nao,Cd,YZl),(Nao,Cd,YZm),(Nao,Cd,YZn),(Nao,Cd,YZo),(Nao,Cd,YZp),(Nao,C d,YZq),(Nao,Cd,YZr),(Nao,Cd,YZs),(Nao,Cd,YZt),(Nao,Cd,YZu),(Nao,Cd,YZy),(Nao,Cd,Y Zw),(Nao,Cd,YZx),(Nao,Cd,YZy),(Nao,Cd,YZz),(Nao,Cd,YZaa),(Nao,Cd,YZab),(Nao,Cd,Y Zac),(Nao,Cd,YZad),(Nao,Cd,YZae),(Nao,Cd,YZaf),(Nao,Cd,YZag),(Nao,Ce,YZa),(Nao,Ce, YZb),(Nao,Ce,YZc),(Nao,Ce,YZd),(Nao,Ce,YZe),(Nao,Ce,YZf),(Nao,Ce,YZg),(Nao,Ce,YZ h),(Nao,Ce,YZi),(Nao,Ce,YZj),(Nao,Ce,YZk),(Nao,Ce,YZl),(Nao,Ce,YZm),(Nao,Ce,YZn),( Nao,Ce,YZo),(Nao,Ce,YZp),(Nao,Ce,YZq),(Nao,Ce,YZr),(Nao,Ce,YZs),(Nao,Ce,YZt),(Nao, Ce,YZu),(Nao,Ce,YZy),(Nao,Ce,YZw),(Nao,Ce,YZx),(Nao,Ce,YZy),(Nao,Ce,YZz),(Nao,Ce ,YZaa),(Nao,Ce,YZab),(Nao,Ce,YZac),(Nao,Ce,YZad),(Nao,Ce,YZae),(Nao,Ce,YZaf),(Nao, Ce,YZag),(Nao,Cf,YZa),(Nao,Cf,YZb),(Nao,Cf,YZc),(Nao,Cf,YZd),(Nao,Cf,YZe),(Nao,Cf, YZf),(Nao,Cf,YZg),(Nao,Cf,YZh),(Nao,Cf,YZi),(Nao,Cf,YZj),(Nao,Cf,YZk),(Nao,Cf,YZl),( Nao,Cf,YZm),(Nao,Cf,YZn),(Nao,Cf,YZo),(Nao,Cf,YZp),(Nao,Cf,YZq),(Nao,Cf,YZr),(Nao, Cf,YZs),(Nao,Cf,YZt),(Nao,Cf,YZu),(Nao,Cf,YZy),(Nao,Cf,YZw),(Nao,Cf,YZx),(Nao,Cf,Y Zy),(Nao,Cf,YZz),(Nao,Cf,YZaa),(Nao,Cf,YZab),(Nao,Cf,YZac),(Nao,Cf,YZad),(Nao,Cf,Y Zae),(Nao,Cf,YZaf),(Nao,Cf,YZag),(Nao,Cg,YZa),(Nao,Cg,YZb),(Nao,Cg,YZc),(Nao,Cg,Y Zd),(Nao,Cg,YZe),(Nao,Cg,YZf),(Nao,Cg,YZg),(Nao,Cg,YZh),(Nao,Cg,YZi),(Nao,Cg,YZj), (Nao,Cg,YZk),(Nao,Cg,YZl),(Nao,Cg,YZm),(Nao,Cg,YZn),(Nao,Cg,YZo),(Nao,Cg,YZp),(N ao,Cg,YZq),(Nao,Cg,YZr),(Nao,Cg,YZs),(Nao,Cg,YZt),(Nao,Cg,YZu),(Nao,Cg,YZy),(Nao, Cg,YZw),(Nao,Cg,YZx),(Nao,Cg,YZy),(Nao,Cg,YZz),(Nao,Cg,YZaa),(Nao,Cg,YZab),(Nao, Cg,YZac),(Nao,Cg,YZad),(Nao,Cg,YZae),(Nao,Cg,YZaf),(Nao,Cg,YZag),(Nao,Ch,YZa),(N ao,Ch,YZb),(Nao,Ch,YZc),(Nao,Ch,YZd),(Nao,Ch,YZe),(Nao,Ch,YZf),(Nao,Ch,YZg),(Nao, Ch,YZh),(Nao,Ch,YZi),(Nao,Ch,YZj),(Nao,Ch,YZk),(Nao,Ch,YZl),(Nao,Ch,YZm),(Nao,Ch, YZn),(Nao,Ch,YZo),(Nao,Ch,YZp),(Nao,Ch,YZq),(Nao,Ch,YZr),(Nao,Ch,YZs),(Nao,Ch,YZ t),(Nao,Ch,YZu),(Nao,Ch,YZy),(Nao,Ch,YZw),(Nao,Ch,YZx),(Nao,Ch,YZy),(Nao,Ch,YZz), (Nao,Ch,YZaa),(Nao,Ch,YZab),(Nao,Ch,YZac),(Nao,Ch,YZad),(Nao,Ch,YZae),(Nao,Ch,YZ af),(Nao,Ch,YZag),(Nao,Ci,YZa),(Nao,Ci,YZb),(Nao,Ci,YZc),(Nao,Ci,YZd),(Nao,Ci,YZe),( Nao,Ci,YZf),(Nao,Ci,YZg),(Nao,Ci,YZh),(Nao,Ci,YZi),(Nao,Ci,YZj),(Nao,Ci,YZk),(Nao,Ci ,YZl),(Nao,Ci,YZm),(Nao,Ci,YZn),(Nao,Ci,YZo),(Nao,Ci,YZp),(Nao,Ci,YZq),(Nao,Ci,YZr) ,(Nao,Ci,YZs),(Nao,Ci,YZt),(Nao,Ci,YZu),(Nao,Ci,YZv),(Nao,Ci,YZw),(Nao,Ci,YZx),(Nao, Ci,YZy),(Nao,Ci,YZz),(Nao,Ci,YZaa),(Nao,Ci,YZab),(Nao,Ci,YZac),(Nao,Ci,YZad),(Nao,C i,YZae),(Nao,Ci,YZaf),(Nao,Ci,YZag),(Nao,Cj,YZa),(Nao,Cj,YZb),(Nao,Cj,YZc),(Nao,Cj,Y Zd),(Nao,Cj,YZe),(Nao,Cj,YZf),(Nao,Cj,YZg),(Nao,Cj,YZh),(Nao,Cj,YZi),(Nao,Cj,YZj),(N ao,Cj,YZk),(Nao,Cj,YZl),(Nao,Cj,YZm),(Nao,Cj,YZn),(Nao,Cj,YZo),(Nao,Cj,YZp),(Nao,Cj, YZq),(Nao,Cj,YZr),(Nao,Cj,YZs),(Nao,Cj,YZt),(Nao,Cj,YZu),(Nao,Cj,YZy),(Nao,Cj,YZw), (Nao,Cj,YZx),(Nao,Cj,YZy),(Nao,Cj,YZz),(Nao,Cj,YZaa),(Nao,Cj,YZab),(Nao,Cj,YZac),(N ao,Cj,YZad),(Nao,Cj,YZae),(Nao,Cj,YZaf),(Nao,Cj,YZag),(Nap,Ca,YZa),(Nap,Ca,YZb),(Na p,Ca,YZc),(Nap,Ca,YZd),(Nap,Ca,YZe),(Nap,Ca,YZf),(Nap,Ca,YZg),(Nap,Ca,YZh),(Nap,C a,YZi),(Nap,Ca,YZj),(Nap,Ca,YZk),(Nap,Ca,YZl),(Nap,Ca,YZm),(Nap,Ca,YZn),(Nap,Ca,Y Zo),(Nap,Ca,YZp),(Nap,Ca,YZq),(Nap,Ca,YZr),(Nap,Ca,YZs),(Nap,Ca,YZt),(Nap,Ca,YZu), (Nap,Ca,YZy),(Nap,Ca,YZw),(Nap,Ca,YZx),(Nap,Ca,YZy),(Nap,Ca,YZz),(Nap,Ca,YZaa),( Nap,Ca,YZab),(Nap,Ca,YZac),(Nap,Ca,YZad),(Nap,Ca,YZae),(Nap,Ca,YZaf),(Nap,Ca,YZa g),(Nap,Cb,YZa),(Nap,Cb,YZb),(Nap,Cb,YZc),(Nap,Cb,YZd),(Nap,Cb,YZe),(Nap,Cb,YZf),( Nap,Cb,YZg),(Nap,Cb,YZh),(Nap,Cb,YZi),(Nap,Cb,YZj),(Nap,Cb,YZk),(Nap,Cb,YZl),(Nap ,Cb,YZm),(Nap,Cb,YZn),(Nap,Cb,YZo),(Nap,Cb,YZp),(Nap,Cb,YZq),(Nap,Cb,YZr),(Nap,C b,YZs),(Nap,Cb,YZt),(Nap,Cb,YZu),(Nap,Cb,YZy),(Nap,Cb,YZw),(Nap,Cb,YZx),(Nap,Cb, YZy),(Nap,Cb,YZz),(Nap,Cb,YZaa),(Nap,Cb,YZab),(Nap,Cb,YZac),(Nap,Cb,YZad),(Nap,C b,YZae),(Nap,Cb,YZaf),(Nap,Cb,YZag),(Nap,Cc,YZa),(Nap,Cc,YZb),(Nap,Cc,YZc),(Nap,C c,YZcl),(Nap,Cc,YZe),(Nap,Cc,YZf),(Nap,Cc,YZg),(Nap,Cc,YZh),(Nap,Cc,YZi),(Nap,Cc,Y Zj),(Nap,Cc,YZk),(Nap,Cc,YZl),(Nap,Cc,YZm),(Nap,Cc,YZn),(Nap,Cc,YZo),(Nap,Cc,YZp) ,(Nap,Cc,YZq),(Nap,Cc,YZr),(Nap,Cc,YZs),(Nap,Cc,YZf),(Nap,Cc,YZu),(Nap,Cc,YZy),(Na p,Cc,YZw),(Nap,Cc,YZx),(Nap,Cc,YZy),(Nap,Cc,YZz),(Nap,Cc,YZaa),(Nap,Cc,YZab),(Na p,Cc,YZac),(Nap,Cc,YZad),(Nap,Cc,YZae),(Nap,Cc,YZaf),(Nap,Cc,YZag),(Nap,Cd,YZa),( Nap,Cd,YZb),(Nap,Cd,YZc),(Nap,Cd,YZd),(Nap,Cd,YZe),(Nap,Cd,YZf),(Nap,Cd,YZg),(Na p,Cd,YZh),(Nap,Cd,YZi),(Nap,Cd,YZj),(Nap,Cd,YZk),(Nap,Cd,YZl),(Nap,Cd,YZm),(Nap,C d,YZn),(Nap,Cd,YZo),(Nap,Cd,YZp),(Nap,Cd,YZq),(Nap,Cd,YZr),(Nap,Cd,YZs),(Nap,Cd, YZt),(Nap,Cd,YZu),(Nap,Cd,YZy),(Nap,Cd,YZw),(Nap,Cd,YZx),(Nap,Cd,YZy),(Nap,Cd,Y Zz),(Nap,Cd,YZaa),(Nap,Cd,YZab),(Nap,Cd,YZac),(Nap,Cd,YZad),(Nap,Cd,YZae),(Nap,Cd ,YZaf),(Nap,Cd,YZag),(Nap,Ce,YZa),(Nap,Ce,YZb),(Nap,Ce,YZc),(Nap,Ce,YZd),(Nap,Ce, YZe),(Nap,Ce,YZf),(Nap,Ce,YZg),(Nap,Ce,YZh),(Nap,Ce,YZi),(Nap,Ce,YZj),(Nap,Ce,YZk) ,(Nap,Ce,YZl),(Nap,Ce,YZm),(Nap,Ce,YZn),(Nap,Ce,YZo),(Nap,Ce,YZp),(Nap,Ce,YZq),( Nap,Ce,YZr),(Nap,Ce,YZs),(Nap,Ce,YZf),(Nap,Ce,YZu),(Nap,Ce,YZy),(Nap,Ce,YZw),(Nap ,Ce,YZx),(Nap,Ce,YZy),(Nap,Ce,YZz),(Nap,Ce,YZaa),(Nap,Ce,YZab),(Nap,Ce,YZac),(Nap, Ce,YZad),(Nap,Ce,YZae),(Nap,Ce,YZaf),(Nap,Ce,YZag),(Nap,Cf,YZa),(Nap,Cf,YZb),(Nap, Cf,YZc),(Nap,Cf,YZd),(Nap,Cf,YZe),(Nap,Cf,YZf),(Nap,Cf,YZg),(Nap,Cf,YZh),(Nap,Cf,Y Zi),(Nap,Cf,YZj),(Nap,Cf,YZk),(Nap,Cf,YZl),(Nap,Cf,YZm),(Nap,Cf,YZn),(Nap,Cf,YZo),( Nap,Cf,YZp),(Nap,Cf,YZq),(Nap,Cf,YZr),(Nap,Cf,YZs),(Nap,Cf,YZf),(Nap,Cf,YZu),(Nap,C f,YZy),(Nap,Cf,YZw),(Nap,Cf,YZx),(Nap,Cf,YZy),(Nap,Cf,YZz),(Nap,Cf,YZaa),(Nap,Cf,Y Zab),(Nap,Cf,YZac),(Nap,Cf,YZad),(Nap,Cf,YZae),(Nap,Cf,YZaf),(Nap,Cf,YZag),(Nap,Cg, YZa),(Nap,Cg,YZb),(Nap,Cg,YZc),(Nap,Cg,YZd),(Nap,Cg,YZe),(Nap,Cg,YZf),(Nap,Cg,YZ g),(Nap,Cg,YZh),(Nap,Cg,YZi),(Nap,Cg,YZj),(Nap,Cg,YZk),(Nap,Cg,YZl),(Nap,Cg,YZm),( Nap,Cg,YZn),(Nap,Cg,YZo),(Nap,Cg,YZp),(Nap,Cg,YZq),(Nap,Cg,YZr),(Nap,Cg,YZs),(Na p,Cg,YZf),(Nap,Cg,YZu),(Nap,Cg,YZv),(Nap,Cg,YZw),(Nap,Cg,YZx),(Nap,Cg,YZy),(Nap, Cg,YZz),(Nap,Cg,YZaa),(Nap,Cg,YZab),(Nap,Cg,YZac),(Nap,Cg,YZad),(Nap,Cg,YZae),(N ap,Cg,YZaf),(Nap,Cg,YZag),(Nap,Ch,YZa),(Nap,Ch,YZb),(Nap,Ch,YZc),(Nap,Ch,YZd),(Na p,Ch,YZe),(Nap,Ch,YZf),(Nap,Ch,YZg),(Nap,Ch,YZh),(Nap,Ch,YZi),(Nap,Ch,YZj),(Nap,C h,YZk),(Nap,Ch,YZl),(Nap,Ch,YZm),(Nap,Ch,YZn),(Nap,Ch,YZo),(Nap,Ch,YZp),(Nap,Ch, YZq),(Nap,Ch,YZr),(Nap,Ch,YZs),(Nap,Ch,YZt),(Nap,Ch,YZu),(Nap,Ch,YZy),(Nap,Ch,YZ w),(Nap,Ch,YZx),(Nap,Ch,YZy),(Nap,Ch,YZz),(Nap,Ch,YZaa),(Nap,Ch,YZab),(Nap,Ch,YZ ac),(Nap,Ch,YZad),(Nap,Ch,YZae),(Nap,Ch,YZaf),(Nap,Ch,YZag),(Nap,Ci,YZa),(Nap,Ci,Y Zb),(Nap,Ci,YZc),(Nap,Ci,YZd),(Nap,Ci,YZe),(Nap,Ci,YZf),(Nap,Ci,YZg),(Nap,Ci,YZh),( Nap,Ci,YZi),(Nap,Ci,YZj),(Nap,Ci,YZk),(Nap,Ci,YZl),(Nap,Ci,YZm),(Nap,Ci,YZn),(Nap,C i,YZo),(Nap,Ci,YZp),(Nap,Ci,YZq),(Nap,Ci,YZr),(Nap,Ci,YZs),(Nap,Ci,YZt),(Nap,Ci,YZu), (Nap,Ci,YZy),(Nap,Ci,YZw),(Nap,Ci,YZx),(Nap,Ci,YZy),(Nap,Ci,YZz),(Nap,Ci,YZaa),(Na p,Ci,YZab),(Nap,Ci,YZac),(Nap,Ci,YZad),(Nap,Ci,YZae),(Nap,Ci,YZaf),(Nap,Ci,YZag),(Na p,Cj,YZa),(Nap,Cj,YZb),(Nap,Cj,YZc),(Nap,Cj,YZd),(Nap,Cj,YZe),(Nap,Cj,YZf),(Nap,Cj,Y Zg),(Nap,Cj,YZh),(Nap,Cj,YZi),(Nap,Cj,YZj),(Nap,Cj,YZk),(Nap,Cj,YZl),(Nap,Cj,YZm),(N ap,Cj,YZn),(Nap,Cj,YZo),(Nap,Cj,YZp),(Nap,Cj,YZq),(Nap,Cj,YZr),(Nap,Cj,YZs),(Nap,Cj, YZt),(Nap,Cj,YZu),(Nap,Cj,YZv),(Nap,Cj,YZw),(Nap,Cj,YZx),(Nap,Cj,YZy),(Nap,Cj,YZz), (Nap,Cj,YZaa),(Nap,Cj,YZab),(Nap,Cj,YZac),(Nap,Cj,YZad),(Nap,Cj,YZae),(Nap,Cj,YZaf), (Nap,Cj,YZag),(Naq,Ca,YZa),(Naq,Ca,YZb),(Naq,Ca,YZc),(Naq,Ca,YZd),(Naq,Ca,YZe),(N aq,Ca,YZf),(Naq,Ca,YZg),(Naq,Ca,YZh),(Naq,Ca,YZi),(Naq,Ca,YZj),(Naq,Ca,YZk),(Naq,C a,YZl),(Naq,Ca,YZm),(Naq,Ca,YZn),(Naq,Ca,YZo),(Naq,Ca,YZp),(Naq,Ca,YZq),(Naq,Ca, YZr),(Naq,Ca,YZs),(Naq,Ca,YZt),(Naq,Ca,YZu),(Naq,Ca,YZy),(Naq,Ca,YZw),(Naq,Ca,YZ x),(Naq,Ca,YZy),(Naq,Ca,YZz),(Naq,Ca,YZaa),(Naq,Ca,YZab),(Naq,Ca,YZac),(Naq,Ca,YZ ad),(Naq,Ca,YZae),(Naq,Ca,YZaf),(Naq,Ca,YZag),(Naq,Cb,YZa),(Naq,Cb,YZb),(Naq,Cb,Y Zc),(Naq,Cb,YZcl),(Naq,Cb,YZe),(Naq,Cb,YZf),(Naq,Cb,YZg),(Naq,Cb,Ya),(Naq,Cb,YZi) ,(Naq,Cb,YZj),(Naq,Cb,YZk),(Naq,Cb,YZl),(Naq,Cb,YZm),(Naq,Cb,YZn),(Naq,Cb,YZo),( Naq,Cb,YZp),(Naq,Cb,YZq),(Naq,Cb,YZr),(Naq,Cb,YZs),(Naq,Cb,YZt),(Naq,Cb,YZu),(Na q,Cb,YZy),(Naq,Cb,YZw),(Naq,Cb,YZx),(Naq,Cb,YZy),(Naq,Cb,YZz),(Naq,Cb,YZaa),(Naq ,Cb,YZab),(Naq,Cb,YZac),(Naq,Cb,YZad),(Naq,Cb,YZae),(Naq,Cb,YZaf),(Naq,Cb,YZag),( Naq,Cc,YZa),(Naq,Cc,YZb),(Naq,Cc,YZc),(Naq,Cc,YZd),(Naq,Cc,YZe),(Naq,Cc,YZf),(Naq ,Cc,YZg),(Naq,Cc,YZh),(Naq,Cc,YZi),(Naq,Cc,YZj),(Naq,Cc,YZk),(Naq,Cc,YZl),(Naq,Cc, YZm),(Naq,Cc,YZn),(Naq,Cc,YZo),(Naq,Cc,YZp),(Naq,Cc,YZq),(Naq,Cc,YZr),(Naq,Cc,YZ s),(Naq,Cc,Ya),(Naq,Cc,YZu),(Naq,Cc,YZy),(Naq,Cc,YZw),(Naq,Cc,YZx),(Naq,Cc,YZy),( Naq,Cc,YZz),(Naq,Cc,YZaa),(Naq,Cc,YZab),(Naq,Cc,YZac),(Naq,Cc,YZad),(Naq,Cc,YZae) ,(Naq,Cc,YZaf),(Naq,Cc,YZag),(Naq,Cd,YZa),(Naq,Cd,YZb),(Naq,Cd,YZc),(Naq,Cd,YZd),( Naq,Cd,YZe),(Naq,Cd,YZf),(Naq,Cd,YZg),(Naq,Cd,YZh),(Naq,Cd,YZi),(Naq,Cd,YZj),(Naq ,Cd,YZk),(Naq,Cd,YZl),(Naq,Cd,YZm),(Naq,Cd,YZn),(Naq,Cd,YZo),(Naq,Cd,YZp),(Naq,C d,YZq),(Naq,Cd,YZr),(Naq,Cd,YZs),(Naq,Cd,YZf),(Naq,Cd,YZu),(Naq,Cd,YZy),(Naq,Cd,Y Zw),(Naq,Cd,YZx),(Naq,Cd,YZy),(Naq,Cd,YZz),(Naq,Cd,YZaa),(Naq,Cd,YZab),(Naq,Cd,Y Zac),(Naq,Cd,YZad),(Naq,Cd,YZae),(Naq,Cd,YZaf),(Naq,Cd,YZag),(Naq,Ce,YZa),(Naq,Ce, YZb),(Naq,Ce,YZc),(Naq,Ce,YZd),(Naq,Ce,YZe),(Naq,Ce,YZf),(Naq,Ce,YZg),(Naq,Ce,YZ h),(Naq,Ce,YZi),(Naq,Ce,YZj),(Naq,Ce,YZk),(Naq,Ce,YZl),(Naq,Ce,YZm),(Naq,Ce,YZn),( Naq,Ce,YZo),(Naq,Ce,YZp),(Naq,Ce,YZq),(Naq,Ce,YZr),(Naq,Ce,YZs),(Naq,Ce,YZt),(Naq, Ce,YZu),(Naq,Ce,YZy),(Naq,Ce,YZw),(Naq,Ce,YZx),(Naq,Ce,YZy),(Naq,Ce,YZz),(Naq,Ce ,YZaa),(Naq,Ce,YZab),(Naq,Ce,YZac),(Naq,Ce,YZad),(Naq,Ce,YZae),(Naq,Ce,YZaf),(Naq, Ce,YZag),(Naq,Cf,YZa),(Naq,Cf,YZb),(Naq,Cf,YZc),(Naq,Cf,YZd),(Naq,Cf,YZe),(Naq,Cf, YZf),(Naq,Cf,YZg),(Naq,Cf,YZh),(Naq,Cf,YZi),(Naq,Cf,YZj),(Naq,Cf,YZk),(Naq,Cf,YZl),( Naq,Cf,YZm),(Naq,Cf,YZn),(Naq,Cf,YZo),(Naq,Cf,YZp),(Naq,Cf,YZq),(Naq,Cf,YZr),(Naq, Cf,YZs),(Naq,Cf,YZt),(Naq,Cf,YZu),(Naq,Cf,YZv),(Naq,Cf,YZw),(Naq,Cf,YZx),(Naq,Cf,Y Zy),(Naq,Cf,YZz),(Naq,Cf,YZaa),(Naq,Cf,YZab),(Naq,Cf,YZac),(Naq,Cf,YZad),(Naq,Cf,Y Zae),(Naq,Cf,YZaf),(Naq,Cf,YZag),(Naq,Cg,YZa),(Naq,Cg,YZb),(Naq,Cg,YZc),(Naq,Cg,Y Zd),(Naq,Cg,YZe),(Naq,Cg,YZf),(Naq,Cg,YZg),(Naq,Cg,YZh),(Naq,Cg,YZi),(Naq,Cg,YZj), (Naq,Cg,YZk),(Naq,Cg,YZl),(Naq,Cg,YZm),(Naq,Cg,YZn),(Naq,Cg,YZo),(Naq,Cg,YZp),(N aq,Cg,YZq),(Naq,Cg,YZr),(Naq,Cg,YZs),(Naq,Cg,YZt),(Naq,Cg,YZu),(Naq,Cg,YZy),(Naq, Cg,YZw),(Naq,Cg,YZx),(Naq,Cg,YZy),(Naq,Cg,YZz),(Naq,Cg,YZaa),(Naq,Cg,YZab),(Naq, Cg,YZac),(Naq,Cg,YZad),(Naq,Cg,YZae),(Naq,Cg,YZaf),(Naq,Cg,YZag),(Naq,Ch,YZa),(N aq,Ch,YZb),(Naq,Ch,YZc),(Naq,Ch,YZd),(Naq,Ch,YZe),(Naq,Ch,YZf),(Naq,Ch,YZg),(Naq, Ch,YZh),(Naq,Ch,YZi),(Naq,Ch,YZj),(Naq,Ch,YZk),(Naq,Ch,YZl),(Naq,Ch,YZm),(Naq,Ch, YZn),(Naq,Ch,YZo),(Naq,Ch,YZp),(Naq,Ch,YZq),(Naq,Ch,YZr),(Naq,Ch,YZs),(Naq,Ch,YZ t),(Naq,Ch,YZu),(Naq,Ch,YZy),(Naq,Ch,YZw),(Naq,Ch,YZx),(Naq,Ch,YZy),(Naq,Ch,YZz), (Naq,Ch,YZaa),(Naq,Ch,YZab),(Naq,Ch,YZac),(Naq,Ch,YZad),(Naq,Ch,YZae),(Naq,Ch,YZ af),(Naq,Ch,YZag),(Naq,Ci,YZa),(Naq,Ci,YZb),(Naq,Ci,YZc),(Naq,Ci,YZd),(Naq,Ci,YZe),( Naq,Ci,YZf),(Naq,Ci,YZg),(Naq,Ci,YZh),(Naq,Ci,YZi),(Naq,Ci,YZj),(Naq,Ci,YZk),(Naq,Ci ,YZl),(Naq,Ci,YZm),(Naq,Ci,YZn),(Naq,Ci,YZo),(Naq,Ci,YZp),(Naq,Ci,YZq),(Naq,Ci,YZr) ,(Naq,Ci,YZs),(Naq,Ci,YZt),(Naq,Ci,YZu),(Naq,Ci,YZNT),(Naq,Ci,YZw),(Naq,Ci,YZx),(Naq, Ci,YZy),(Naq,Ci,YZz),(Naq,Ci,YZaa),(Naq,Ci,YZab),(Naq,Ci,YZac),(Naq,Ci,YZad),(Naq,C i,YZae),(Naq,Ci,YZaf),(Naq,Ci,YZag),(Naq,Cj,YZa),(Naq,Cj,YZb),(Naq,Cj,YZc),(Naq,Cj,Y Zd),(Naq,Cj,YZe),(Naq,Cj,YZf),(Naq,Cj,YZg),(Naq,Cj,YZh),(Naq,Cj,YZi),(Naq,Cj,YZj),(N aq,Cj,YZk),(Naq,Cj,YZl),(Naq,Cj,YZm),(Naq,Cj,YZn),(Naq,Cj,YZo),(Naq,Cj,YZp),(Naq,Cj, YZq),(Naq,Cj,YZr),(Naq,Cj,YZs),(Naq,Cj,YZt),(Naq,Cj,YZu),(Naq,Cj,YZy),(Naq,Cj,YZw), (Naq,Cj,YZx),(Naq,Cj,YZy),(Naq,Cj,YZz),(Naq,Cj,YZaa),(Naq,Cj,YZab),(Naq,Cj,YZac),(N aq,Cj,YZad),(Naq,Cj,YZae),(Naq,Cj,YZaf),(Naq,Cj,YZag),(Nar,Ca,YZa),(Nar,Ca,YZb),(Nar ,Ca,YZc),(Nar,Ca,YZd),(Nar,Ca,YZe),(Nar,Ca,YZf),(Nar,Ca,YZg),(Nar,Ca,YZh),(Nar,Ca,Y Zi),(Nar,Ca,YZj),(Nar,Ca,YZk),(Nar,Ca,YZl),(Nar,Ca,YZm),(Nar,Ca,YZn),(Nar,Ca,YZo),(N ar,Ca,YZp),(Nar,Ca,YZq),(Nar,Ca,YZr),(Nar,Ca,YZs),(Nar,Ca,YZt),(Nar,Ca,YZu),(Nar,Ca, YZ-v),(Nar,Ca,YZw),(Nar,Ca,YZx),(Nar,Ca,YZy),(Nar,Ca,YZz),(Nar,Ca,YZaa),(Nar,Ca,YZa b),(Nar,Ca,YZac),(Nar,Ca,YZad),(Nar,Ca,YZae),(Nar,Ca,YZaf),(Nar,Ca,YZag),(Nar,Cb,YZa) ,(Nar,Cb,YZb),(Nar,Cb,YZc),(Nar,Cb,YZd),(Nar,Cb,YZe),(Nar,Cb,YZf),(Nar,Cb,YZg),(Na r,Cb,YZh),(Nar,Cb,YZi),(Nar,Cb,YZj),(Nar,Cb,YZk),(Nar,Cb,YZl),(Nar,Cb,YZm),(Nar,Cb, YZn),(Nar,Cb,YZo),(Nar,Cb,YZp),(Nar,Cb,YZq),(Nar,Cb,YZr),(Nar,Cb,YZs),(Nar,Cb,YZt), (Nar,Cb,YZu),(Nar,Cb,YZy),(Nar,Cb,YZw),(Nar,Cb,YZx),(Nar,Cb,YZy),(Nar,Cb,YZz),(Nar ,Cb,YZaa),(Nar,Cb,YZab),(Nar,Cb,YZac),(Nar,Cb,YZad),(Nar,Cb,YZae),(Nar,Cb,YZaf),(Na r,Cb,YZag),(Nar,Cc,YZa),(Nar,Cc,YZb),(Nar,Cc,YZc),(Nar,Cc,YZd),(Nar,Cc,YZe),(Nar,Cc, YZf),(Nar,Cc,YZg),(Nar,Cc,YZh),(Nar,Cc,YZi),(Nar,Cc,YZj),(Nar,Cc,YZk),(Nar,Cc,YZl),( Nar,Cc,YZm),(Nar,Cc,YZn),(Nar,Cc,YZo),(Nar,Cc,YZp),(Nar,Cc,YZq),(Nar,Cc,YZr),(Nar, Cc,YZs),(Nar,Cc,YZt),(Nar,Cc,YZu),(Nar,Cc,YZy),(Nar,Cc,YZw),(Nar,Cc,YZx),(Nar,Cc,Y Zy),(Nar,Cc,YZz),(Nar,Cc,YZaa),(Nar,Cc,YZab),(Nar,Cc,YZac),(Nar,Cc,YZad),(Nar,Cc,YZ ae),(Nar,Cc,YZaf),(Nar,Cc,YZag),(Nar,Cd,YZa),(Nar,Cd,YZb),(Nar,Cd,YZc),(Nar,Cd,YZd), (Nar,Cd,YZe),(Nar,Cd,YZf),(Nar,Cd,YZg),(Nar,Cd,YZh),(Nar,Cd,YZi),(Nar,Cd,YZj),(Nar,C d,YZk),(Nar,Cd,YZl),(Nar,Cd,YZm),(Nar,Cd,YZn),(Nar,Cd,YZo),(Nar,Cd,YZp),(Nar,Cd,Y Zq),(Nar,Cd,YZr),(Nar,Cd,YZs),(Nar,Cd,YZt),(Nar,Cd,YZu),(Nar,Cd,YZy),(Nar,Cd,YZw),( Nar,Cd,YZx),(Nar,Cd,YZy),(Nar,Cd,YZz),(Nar,Cd,YZaa),(Nar,Cd,YZab),(Nar,Cd,YZac),(N ar,Cd,YZad),(Nar,Cd,YZae),(Nar,Cd,YZaf),(Nar,Cd,YZag),(Nar,Ce,YZa),(Nar,Ce,YZb),(Na r,Ce,YZc),(Nar,Ce,YZd),(Nar,Ce,YZe),(Nar,Ce,YZf),(Nar,Ce,YZg),(Nar,Ce,YZh),(Nar,Ce,Y Zi),(Nar,Ce,YZj),(Nar,Ce,YZk),(Nar,Ce,YZl),(Nar,Ce,YZm),(Nar,Ce,YZn),(Nar,Ce,YZo),(N ar,Ce,YZp),(Nar,Ce,YZq),(Nar,Ce,YZr),(Nar,Ce,YZs),(Nar,Ce,YZt),(Nar,Ce,YZu),(Nar,Ce, YZy),(Nar,Ce,YZw),(Nar,Ce,YZx),(Nar,Ce,YZy),(Nar,Ce,YZz),(Nar,Ce,YZaa),(Nar,Ce,YZa b),(Nar,Ce,YZac),(Nar,Ce,YZad),(Nar,Ce,YZae),(Nar,Ce,YZaf),(Nar,Ce,YZag),(Nar,Cf,YZa) ,(Nar,Cf,YZb),(Nar,Cf,YZc),(Nar,Cf,YZd),(Nar,Cf,YZe),(Nar,Cf,YZf),(Nar,Cf,YZg),(Nar,C f,YZh),(Nar,Cf,YZi),(Nar,Cf,YZj),(Nar,Cf,YZk),(Nar,Cf,YZl),(Nar,Cf,YZm),(Nar,Cf,YZn),( Nar,Cf,YZo),(Nar,Cf,YZp),(Nar,Cf,YZq),(Nar,Cf,YZr),(Nar,Cf,YZs),(Nar,Cf,YZf),(Nar,Cf, YZu),(Nar,Cf,YZv),(Nar,Cf,YZw),(Nar,Cf,YZx),(Nar,Cf,YZy),(Nar,Cf,YZz),(Nar,Cf,YZaa), (Nar,Cf,YZab),(Nar,Cf,YZac),(Nar,Cf,YZad),(Nar,Cf,YZae),(Nar,Cf,YZaf),(Nar,Cf,YZag),( Nar,Cg,YZa),(Nar,Cg,YZb),(Nar,Cg,YZc),(Nar,Cg,YZd),(Nar,Cg,YZe),(Nar,Cg,YZf),(Nar,C g,YZg),(Nar,Cg,YZh),(Nar,Cg,YZi),(Nar,Cg,YZj),(Nar,Cg,YZk),(Nar,Cg,YZl),(Nar,Cg,YZ m),(Nar,Cg,YZn),(Nar,Cg,YZo),(Nar,Cg,YZp),(Nar,Cg,YZq),(Nar,Cg,YZr),(Nar,Cg,YZs),( Nar,Cg,YZf),(Nar,Cg,YZu),(Nar,Cg,YZy),(Nar,Cg,YZw),(Nar,Cg,YZx),(Nar,Cg,YZy),(Nar, Cg,YZz),(Nar,Cg,YZaa),(Nar,Cg,YZab),(Nar,Cg,YZac),(Nar,Cg,YZad),(Nar,Cg,YZae),(Nar, Cg,YZaf),(Nar,Cg,YZag),(Nar,Ch,YZa),(Nar,Ch,YZb),(Nar,Ch,YZc),(Nar,Ch,YZd),(Nar,Ch, YZe),(Nar,Ch,YZf),(Nar,Ch,YZg),(Nar,Ch,YZh),(Nar,Ch,YZi),(Nar,Ch,YZj),(Nar,Ch,YZk),( Nar,Ch,YZl),(Nar,Ch,YZm),(Nar,Ch,YZn),(Nar,Ch,YZo),(Nar,Ch,YZp),(Nar,Ch,YZq),(Nar, Ch,YZr),(Nar,Ch,YZs),(Nar,Ch,Ya),(Nar,Ch,YZu),(Nar,Ch,YZy),(Nar,Ch,YZw),(Nar,Ch,Y Zx),(Nar,Ch,YZy),(Nar,Ch,YZz),(Nar,Ch,YZaa),(Nar,Ch,YZab),(Nar,Ch,YZac),(Nar,Ch,YZ ad),(Nar,Ch,YZae),(Nar,Ch,YZaf),(Nar,Ch,YZag),(Nar,Ci,YZa),(Nar,Ci,YZb),(Nar,Ci,YZc), (Nar,Ci,YZd),(Nar,Ci,YZe),(Nar,Ci,YZf),(Nar,Ci,YZg),(Nar,Ci,YZh),(Nar,Ci,YZi),(Nar,Ci, YZj),(Nar,Ci,YZk),(Nar,Ci,YZl),(Nar,Ci,YZm),(Nar,Ci,YZn),(Nar,Ci,YZo),(Nar,Ci,YZp),(N ar,Ci,YZq),(Nar,Ci,YZr),(Nar,Ci,YZs),(Nar,Ci,YZt),(Nar,Ci,YZu),(Nar,Ci,YZv),(Nar,Ci,YZ w),(Nar,Ci,YZx),(Nar,Ci,YZy),(Nar,Ci,YZz),(Nar,Ci,YZaa),(Nar,Ci,YZab),(Nar,Ci,YZac),( Nar,Ci,YZad),(Nar,Ci,YZae),(Nar,Ci,YZaf),(Nar,Ci,YZag),(Nar,Cj,YZa),(Nar,Cj,YZb),(Nar, Cj,YZc),(Nar,Cj,YZd),(Nar,Cj,YZe),(Nar,Cj,YZf),(Nar,Cj,YZg),(Nar,Cj,YZh),(Nar,Cj,YZi),( Nar,Cj,YZj),(Nar,Cj,YZk),(Nar,Cj,YZl),(Nar,Cj,YZm),(Nar,Cj,YZn),(Nar,Cj,YZo),(Nar,Cj, YZp),(Nar,Cj,YZq),(Nar,Cj,YZr),(Nar,Cj,YZs),(Nar,Cj,YZt),(Nar,Cj,YZu),(Nar,Cj,YZv),(Na r,Cj,YZw),(Nar,Cj,YZx),(Nar,Cj,YZy),(Nar,Cj,YZz),(Nar,Cj,YZaa),(Nar,Cj,YZab),(Nar,Cj, YZac),(Nar,Cj,YZad),(Nar,Cj,YZae),(Nar,Cj,YZaf),(Nar,Cj,YZag),(Nas,Ca,YZa),(Nas,Ca,Y Zb),(Nas,Ca,YZc),(Nas,Ca,YZd),(Nas,Ca,YZe),(Nas,Ca,YZf),(Nas,Ca,YZg),(Nas,Ca,YZh),( Nas,Ca,YZi),(Nas,Ca,YZj),(Nas,Ca,YZk),(Nas,Ca,YZl),(Nas,Ca,YZm),(Nas,Ca,YZn),(Nas,C a,YZo),(Nas,Ca,YZp),(Nas,Ca,YZq),(Nas,Ca,YZr),(Nas,Ca,YZs),(Nas,Ca,YZt),(Nas,Ca,YZu) ,(Nas,Ca,YZv),(Nas,Ca,YZw),(Nas,Ca,YZx),(Nas,Ca,YZy),(Nas,Ca,YZz),(Nas,Ca,YZaa),( Nas,Ca,YZab),(Nas,Ca,YZac),(Nas,Ca,YZad),(Nas,Ca,YZae),(Nas,Ca,YZaf),(Nas,Ca,YZag), (Nas,Cb,YZa),(Nas,Cb,YZb),(Nas,Cb,YZc),(Nas,Cb,YZd),(Nas,Cb,YZe),(Nas,Cb,YZf),(Nas ,Cb,YZg),(Nas,Cb,YZh),(Nas,Cb,YZi),(Nas,Cb,YZj),(Nas,Cb,YZk),(Nas,Cb,YZl),(Nas,Cb,Y Zm),(Nas,Cb,YZn),(Nas,Cb,YZo),(Nas,Cb,YZp),(Nas,Cb,YZq),(Nas,Cb,YZr),(Nas,Cb,YZs), (Nas,Cb,YZt),(Nas,Cb,YZu),(Nas,Cb,YZv),(Nas,Cb,YZw),(Nas,Cb,YZx),(Nas,Cb,YZy),(Na s,Cb,YZz),(Nas,Cb,YZaa),(Nas,Cb,YZab),(Nas,Cb,YZac),(Nas,Cb,YZad),(Nas,Cb,YZae),(N as,Cb,YZaf),(Nas,Cb,YZag),(Nas,Cc,YZa),(Nas,Cc,YZb),(Nas,Cc,YZc),(Nas,Cc,YZd),(Nas, Cc,YZe),(Nas,Cc,YZf),(Nas,Cc,YZg),(Nas,Cc,YZh),(Nas,Cc,YZi),(Nas,Cc,YZj),(Nas,Cc,YZ k),(Nas,Cc,YZl),(Nas,Cc,YZm),(Nas,Cc,YZn),(Nas,Cc,YZo),(Nas,Cc,YZp),(Nas,Cc,YZq),( Nas,Cc,YZr),(Nas,Cc,YZs),(Nas,Cc,YZt),(Nas,Cc,YZu),(Nas,Cc,YZv),(Nas,Cc,YZw),(Nas, Cc,YZx),(Nas,Cc,YZy),(Nas,Cc,YZz),(Nas,Cc,YZaa),(Nas,Cc,YZab),(Nas,Cc,YZac),(Nas,C c,YZad),(Nas,Cc,YZae),(Nas,Cc,YZaf),(Nas,Cc,YZag),(Nas,Cd,YZa),(Nas,Cd,YZb),(Nas,Cd ,YZc),(Nas,Cd,YZd),(Nas,Cd,YZe),(Nas,Cd,YZf),(Nas,Cd,YZg),(Nas,Cd,YZh),(Nas,Cd,YZi) ,(Nas,Cd,YZj),(Nas,Cd,YZk),(Nas,Cd,YZl),(Nas,Cd,YZm),(Nas,Cd,YZn),(Nas,Cd,YZo),(N as,Cd,YZp),(Nas,Cd,YZq),(Nas,Cd,YZr),(Nas,Cd,YZs),(Nas,Cd,YZt),(Nas,Cd,YZu),(Nas,Cd ,YZv),(Nas,Cd,YZw),(Nas,Cd,YZx),(Nas,Cd,YZy),(Nas,Cd,YZz),(Nas,Cd,YZaa),(Nas,Cd,Y Zab),(Nas,Cd,YZac),(Nas,Cd,YZad),(Nas,Cd,YZae),(Nas,Cd,YZaf),(Nas,Cd,YZag),(Nas,Ce, YZa),(Nas,Ce,YZb),(Nas,Ce,YZc),(Nas,Ce,YZd),(Nas,Ce,YZe),(Nas,Ce,YZf),(Nas,Ce,YZg), (Nas,Ce,YZh),(Nas,Ce,YZi),(Nas,Ce,YZj),(Nas,Ce,YZk),(Nas,Ce,YZl),(Nas,Ce,YZm),(Nas, Ce,YZn),(Nas,Ce,YZo),(Nas,Ce,YZp),(Nas,Ce,YZq),(Nas,Ce,YZr),(Nas,Ce,YZs),(Nas,Ce,Y Zt),(Nas,Ce,YZu),(Nas,Ce,YZv),(Nas,Ce,YZw),(Nas,Ce,YZx),(Nas,Ce,YZy),(Nas,Ce,YZz),( Nas,Ce,YZaa),(Nas,Ce,YZab),(Nas,Ce,YZac),(Nas,Ce,YZad),(Nas,Ce,YZae),(Nas,Ce,YZaf), (Nas,Ce,YZag),(Nas,Cf,YZa),(Nas,Cf,YZb),(Nas,Cf,YZc),(Nas,Cf,YZd),(Nas,Cf,YZe),(Nas, Cf,YZf),(Nas,Cf,YZg),(Nas,Cf,YZh),(Nas,Cf,YZi),(Nas,Cf,YZj),(Nas,Cf,YZk),(Nas,Cf,YZl) ,(Nas,Cf,YZm),(Nas,Cf,YZn),(Nas,Cf,YZo),(Nas,Cf,YZp),(Nas,Cf,YZq),(Nas,Cf,YZr),(Nas, Cf,YZs),(Nas,Cf,YZt),(Nas,Cf,YZu),(Nas,Cf,YZv),(Nas,Cf,YZw),(Nas,Cf,YZx),(Nas,Cf,YZ y),(Nas,Cf,YZz),(Nas,Cf,YZaa),(Nas,Cf,YZab),(Nas,Cf,YZac),(Nas,Cf,YZad),(Nas,Cf,YZae) ,(Nas,Cf,YZaf),(Nas,Cf,YZag),(Nas,Cg,YZa),(Nas,Cg,YZb),(Nas,Cg,YZc),(Nas,Cg,YZd),( Nas,Cg,YZe),(Nas,Cg,YZf),(Nas,Cg,YZg),(Nas,Cg,YZh),(Nas,Cg,YZi),(Nas,Cg,YZj),(Nas, Cg,YZk),(Nas,Cg,YZl),(Nas,Cg,YZm),(Nas,Cg,YZn),(Nas,Cg,YZo),(Nas,Cg,YZp),(Nas,Cg, YZq),(Nas,Cg,YZr),(Nas,Cg,YZs),(Nas,Cg,YZt),(Nas,Cg,YZu),(Nas,Cg,YZv),(Nas,Cg,YZw) ,(Nas,Cg,YZx),(Nas,Cg,YZy),(Nas,Cg,YZz),(Nas,Cg,YZaa),(Nas,Cg,YZab),(Nas,Cg,YZac), (Nas,Cg,YZad),(Nas,Cg,YZae),(Nas,Cg,YZaf),(Nas,Cg,YZag),(Nas,Ch,YZa),(Nas,Ch,YZb), (Nas,Ch,YZc),(Nas,Ch,YZd),(Nas,Ch,YZe),(Nas,Ch,YZf),(Nas,Ch,YZg),(Nas,Ch,YZh),(Nas ,Ch,YZi),(Nas,Ch,YZj),(Nas,Ch,YZk),(Nas,Ch,YZl),(Nas,Ch,YZm),(Nas,Ch,YZn),(Nas,Ch, YZo),(Nas,Ch,YZp),(Nas,Ch,YZq),(Nas,Ch,YZr),(Nas,Ch,YZs),(Nas,Ch,YZt),(Nas,Ch,YZu) ,(Nas,Ch,YZv),(Nas,Ch,YZw),(Nas,Ch,YZx),(Nas,Ch,YZy),(Nas,Ch,YZz),(Nas,Ch,YZaa),( Nas,Ch,YZab),(Nas,Ch,YZac),(Nas,Ch,YZad),(Nas,Ch,YZae),(Nas,Ch,YZaf),(Nas,Ch,YZag) ,(Nas,Ci,YZa),(Nas,Ci,YZb),(Nas,Ci,YZc),(Nas,Ci,YZd),(Nas,Ci,YZe),(Nas,Ci,YZf),(Nas,C i,YZg),(Nas,Ci,YZh),(Nas,Ci,YZi),(Nas,Ci,YZj),(Nas,Ci,Ya),(Nas,Ci,YZl),(Nas,Ci,YZm),( Nas,Ci,YZn),(Nas,Ci,YZo),(Nas,Ci,YZp),(Nas,Ci,YZq),(Nas,Ci,YZr),(Nas,Ci,YZs),(Nas,Ci, YZt),(Nas,Ci,YZu),(Nas,Ci,YZv),(Nas,Ci,YZw),(Nas,Ci,YZx),(Nas,Ci,YZy),(Nas,Ci,YZz),( Nas,Ci,YZaa),(Nas,Ci,YZab),(Nas,Ci,YZac),(Nas,Ci,YZad),(Nas,Ci,YZae),(Nas,Ci,YZaf),(N as,Ci,YZag),(Nas,Cj,YZa),(Nas,Cj,YZb),(Nas,Cj,YZc),(Nas,Cj,YZd),(Nas,Cj,YZe),(Nas,Cj, YZf),(Nas,Cj,YZg),(Nas,Cj,YZh),(Nas,Cj,YZi),(Nas,Cj,YZj),(Nas,Cj,YZk),(Nas,Cj,YZl),(Na s,Cj,YZm),(Nas,Cj,YZn),(Nas,Cj,YZo),(Nas,Cj,YZp),(Nas,Cj,YZq),(Nas,Cj,YZr),(Nas,Cj,Y Zs),(Nas,Cj,YZt),(Nas,Cj,YZu),(Nas,Cj,YZv),(Nas,Cj,YZw),(Nas,Cj,YZx),(Nas,Cj,YZy),(Na s,Cj,YZz),(Nas,Cj,YZaa),(Nas,Cj,YZab),(Nas,Cj,YZac),(Nas,Cj,YZad),(Nas,Cj,YZae),(Nas, Cj,YZaf),(Nas,Cj,YZag),(Nat,Ca,YZa),(Nat,Ca,YZb),(Nat,Ca,YZc),(Nat,Ca,YZd),(Nat,Ca,Y Ze),(Nat,Ca,YZf),(Nat,Ca,YZg),(Nat,Ca,YZh),(Nat,Ca,YZi),(Nat,Ca,YZj),(Nat,Ca,YZk),(Na t,Ca,YZl),(Nat,Ca,YZm),(Nat,Ca,YZn),(Nat,Ca,YZo),(Nat,Ca,YZp),(Nat,Ca,YZq),(Nat,Ca,Y Zr),(Nat,Ca,YZs),(Nat,Ca,YZt),(Nat,Ca,YZu),(Nat,Ca,YZv),(Nat,Ca,YZw),(Nat,Ca,YZx),(N at,Ca,YZy),(Nat,Ca,YZz),(Nat,Ca,YZaa),(Nat,Ca,YZab),(Nat,Ca,YZac),(Nat,Ca,YZad),(Nat, Ca,YZae),(Nat,Ca,YZaf),(Nat,Ca,YZag),(Nat,Cb,YZa),(Nat,Cb,YZb),(Nat,Cb,YZc),(Nat,Cb, YZd),(Nat,Cb,YZe),(Nat,Cb,YZf),(Nat,Cb,YZg),(Nat,Cb,YZh),(Nat,Cb,YZi),(Nat,Cb,YZj),( Nat,Cb,YZk),(Nat,Cb,YZl),(Nat,Cb,YZm),(Nat,Cb,YZn),(Nat,Cb,YZo),(Nat,Cb,YZp),(Nat,C b,YZq),(Nat,Cb,YZr),(Nat,Cb,YZs),(Nat,Cb,YZt),(Nat,Cb,YZu),(Nat,Cb,YZv),(Nat,Cb,YZw) ,(Nat,Cb,YZx),(Nat,Cb,YZy),(Nat,Cb,YZz),(Nat,Cb,YZaa),(Nat,Cb,YZab),(Nat,Cb,YZac),( Nat,Cb,YZad),(Nat,Cb,YZae),(Nat,Cb,YZaf),(Nat,Cb,YZag),(Nat,Cc,YZa),(Nat,Cc,YZb),(N at,Cc,YZc),(Nat,Cc,YZd),(Nat,Cc,YZe),(Nat,Cc,YZf),(Nat,Cc,YZg),(Nat,Cc,YZh),(Nat,Cc,Y Zi),(Nat,Cc,YZj),(Nat,Cc,YZk),(Nat,Cc,YZl),(Nat,Cc,YZm),(Nat,Cc,YZn),(Nat,Cc,YZo),(N at,Cc,YZp),(Nat,Cc,YZq),(Nat,Cc,YZr),(Nat,Cc,YZs),(Nat,Cc,YZt),(Nat,Cc,YZu),(Nat,Cc,Y Zv),(Nat,Cc,YZw),(Nat,Cc,YZx),(Nat,Cc,YZy),(Nat,Cc,YZz),(Nat,Cc,YZaa),(Nat,Cc,YZab), (Nat,Cc,YZac),(Nat,Cc,YZad),(Nat,Cc,YZae),(Nat,Cc,YZaf),(Nat,Cc,YZag),(Nat,Cd,YZa),( Nat,Cd,YZb),(Nat,Cd,YZc),(Nat,Cd,YZd),(Nat,Cd,YZe),(Nat,Cd,YZf),(Nat,Cd,YZg),(Nat,C d,YZh),(Nat,Cd,YZi),(Nat,Cd,YZj),(Nat,Cd,YZk),(Nat,Cd,YZl),(Nat,Cd,YZm),(Nat,Cd,YZn) ,(Nat,Cd,YZo),(Nat,Cd,YZp),(Nat,Cd,YZq),(Nat,Cd,YZr),(Nat,Cd,YZs),(Nat,Cd,YZt),(Nat, Cd,YZu),(Nat,Cd,YZv),(Nat,Cd,YZw),(Nat,Cd,YZx),(Nat,Cd,YZy),(Nat,Cd,YZz),(Nat,Cd,Y Zaa),(Nat,Cd,YZab),(Nat,Cd,YZac),(Nat,Cd,YZad),(Nat,Cd,YZae),(Nat,Cd,YZaf),(Nat,Cd,Y Zag),(Nat,Ce,YZa),(Nat,Ce,YZb),(Nat,Ce,YZc),(Nat,Ce,YZd),(Nat,Ce,YZe),(Nat,Ce,YZf),( Nat,Ce,YZg),(Nat,Ce,YZh),(Nat,Ce,YZi),(Nat,Ce,YZj),(Nat,Ce,YZk),(Nat,Ce,YZl),(Nat,Ce, YZm),(Nat,Ce,YZn),(Nat,Ce,YZo),(Nat,Ce,YZp),(Nat,Ce,YZq),(Nat,Ce,YZr),(Nat,Ce,YZs),( Nat,Ce,YZt),(Nat,Ce,YZu),(Nat,Ce,YZv),(Nat,Ce,YZw),(Nat,Ce,YZx),(Nat,Ce,YZy),(Nat,C e,YZz),(Nat,Ce,YZaa),(Nat,Ce,YZab),(Nat,Ce,YZac),(Nat,Ce,YZad),(Nat,Ce,YZae),(Nat,Ce, YZaf),(Nat,Ce,YZag),(Nat,Cf,YZa),(Nat,Cf,YZb),(Nat,Cf,YZc),(Nat,Cf,YZd),(Nat,Cf,YZe),( Nat,Cf,Ya),(Nat,Cf,YZg),(Nat,Cf,YZh),(Nat,Cf,YZi),(Nat,Cf,YZj),(Nat,Cf,YZk),(Nat,Cf,Y Zl),(Nat,Cf,YZm),(Nat,Cf,YZn),(Nat,Cf,YZo),(Nat,Cf,YZp),(Nat,Cf,YZq),(Nat,Cf,YZr),(Nat ,Cf,YZs),(Nat,Cf,YZt),(Nat,Cf,YZu),(Nat,Cf,YZv),(Nat,Cf,YZw),(Nat,Cf,YZx),(Nat,Cf,YZy) ,(Nat,Cf,YZz),(Nat,Cf,YZaa),(Nat,Cf,YZab),(Nat,Cf,YZac),(Nat,Cf,YZad),(Nat,Cf,YZae),( Nat,Cf,YZaf),(Nat,Cf,YZag),(Nat,Cg,YZa),(Nat,Cg,YZb),(Nat,Cg,YZc),(Nat,Cg,YZd),(Nat, Cg,YZe),(Nat,Cg,YZf),(Nat,Cg,YZg),(Nat,Cg,YZh),(Nat,Cg,YZi),(Nat,Cg,YZj),(Nat,Cg,YZ k),(Nat,Cg,YZl),(Nat,Cg,YZm),(Nat,Cg,YZn),(Nat,Cg,YZo),(Nat,Cg,YZp),(Nat,Cg,YZq),(N at,Cg,YZr),(Nat,Cg,YZs),(Nat,Cg,YZt),(Nat,Cg,YZu),(Nat,Cg,YZv),(Nat,Cg,YZw),(Nat,Cg, YZx),(Nat,Cg,YZy),(Nat,Cg,YZz),(Nat,Cg,YZaa),(Nat,Cg,YZab),(Nat,Cg,YZac),(Nat,Cg,Y Zad),(Nat,Cg,YZae),(Nat,Cg,YZaf),(Nat,Cg,YZag),(Nat,Ch,YZa),(Nat,Ch,YZb),(Nat,Ch,YZ c),(Nat,Ch,YZd),(Nat,Ch,YZe),(Nat,Ch,YZf),(Nat,Ch,YZg),(Nat,Ch,YZh),(Nat,Ch,YZi),(Nat ,Ch,YZj),(Nat,Ch,YZk),(Nat,Ch,YZl),(Nat,Ch,YZm),(Nat,Ch,YZn),(Nat,Ch,YZo),(Nat,Ch,Y Zp),(Nat,Ch,YZq),(Nat,Ch,YZr),(Nat,Ch,YZs),(Nat,Ch,YZt),(Nat,Ch,YZu),(Nat,Ch,YZv),(N at,Ch,YZw),(Nat,Ch,YZx),(Nat,Ch,YZy),(Nat,Ch,YZz),(Nat,Ch,YZaa),(Nat,Ch,YZab),(Nat, Ch,YZac),(Nat,Ch,YZad),(Nat,Ch,YZae),(Nat,Ch,YZaf),(Nat,Ch,YZag),(Nat,Ci,YZa),(Nat,C i,YZb),(Nat,Ci,YZc),(Nat,Ci,YZd),(Nat,Ci,YZe),(Nat,Ci,YZf),(Nat,Ci,YZg),(Nat,Ci,YZh),(N at,Ci,YZi),(Nat,Ci,YZj),(Nat,Ci,YZk),(Nat,Ci,YZl),(Nat,Ci,YZm),(Nat,Ci,YZn),(Nat,Ci,YZo) ,(Nat,Ci,YZp),(Nat,Ci,YZq),(Nat,Ci,YZr),(Nat,Ci,YZs),(Nat,Ci,YZt),(Nat,Ci,YZu),(Nat,Ci, YZv),(Nat,Ci,YZw),(Nat,Ci,YZx),(Nat,Ci,YZy),(Nat,Ci,YZz),(Nat,Ci,YZaa),(Nat,Ci,YZab),( Nat,Ci,YZac),(Nat,Ci,YZad),(Nat,Ci,YZae),(Nat,Ci,YZaf),(Nat,Ci,YZag),(Nat,Cj,YZa),(Nat, Cj,YZb),(Nat,Cj,YZc),(Nat,Cj,YZd),(Nat,Cj,YZe),(Nat,Cj,YZf),(Nat,Cj,YZg),(Nat,Cj,YZh),( Nat,Cj,YZi),(Nat,Cj,YZj),(Nat,Cj,YZk),(Nat,Cj,YZl),(Nat,Cj,YZm),(Nat,Cj,YZn),(Nat,Cj,Y Zo),(Nat,Cj,YZp),(Nat,Cj,YZq),(Nat,Cj,YZr),(Nat,Cj,YZs),(Nat,Cj,YZt),(Nat,Cj,YZu),(Nat, Cj,YZv),(Nat,Cj,YZw),(Nat,Cj,YZx),(Nat,Cj,YZy),(Nat,Cj,YZz),(Nat,Cj,YZaa),(Nat,Cj,YZa b),(Nat,Cj,YZac),(Nat,Cj,YZad),(Nat,Cj,YZae),(Nat,Cj,YZaf),(Nat,Cj,YZag),(Nau,Ca,YZa),( Nau,Ca,YZb),(Nau,Ca,YZc),(Nau,Ca,YZd),(Nau,Ca,YZe),(Nau,Ca,YZf),(Nau,Ca,YZg),(Nau ,Ca,YZh),(Nau,Ca,YZi),(Nau,Ca,YZj),(Nau,Ca,YZk),(Nau,Ca,YZl),(Nau,Ca,YZm),(Nau,Ca, YZn),(Nau,Ca,YZo),(Nau,Ca,YZp),(Nau,Ca,YZq),(Nau,Ca,YZr),(Nau,Ca,YZs),(Nau,Ca,YZt) ,(Nau,Ca,YZu),(Nau,Ca,YZv),(Nau,Ca,YZw),(Nau,Ca,YZx),(Nau,Ca,YZy),(Nau,Ca,YZz),( Nau,Ca,YZaa),(Nau,Ca,YZab),(Nau,Ca,YZac),(Nau,Ca,YZad),(Nau,Ca,YZae),(Nau,Ca,YZaf) ,(Nau,Ca,YZag),(Nau,Cb,YZa),(Nau,Cb,YZb),(Nau,Cb,YZc),(Nau,Cb,YZd),(Nau,Cb,YZe), (Nau,Cb,YZf),(Nau,Cb,YZg),(Nau,Cb,YZh),(Nau,Cb,YZi),(Nau,Cb,YZj),(Nau,Cb,YZk),(Na u,Cb,YZl),(Nau,Cb,YZm),(Nau,Cb,YZn),(Nau,Cb,YZo),(Nau,Cb,YZp),(Nau,Cb,YZq),(Nau, Cb,YZr),(Nau,Cb,YZs),(Nau,Cb,YZt),(Nau,Cb,YZu),(Nau,Cb,YZv),(Nau,Cb,YZw),(Nau,Cb ,YZx),(Nau,Cb,YZy),(Nau,Cb,YZz),(Nau,Cb,YZaa),(Nau,Cb,YZab),(Nau,Cb,YZac),(Nau,C b,YZad),(Nau,Cb,YZae),(Nau,Cb,YZaf),(Nau,Cb,YZag),(Nau,Cc,YZa),(Nau,Cc,YZb),(Nau, Cc,YZc),(Nau,Cc,YZd),(Nau,Cc,YZe),(Nau,Cc,YZf),(Nau,Cc,YZg),(Nau,Cc,YZh),(Nau,Cc, YZi),(Nau,Cc,YZj),(Nau,Cc,YZk),(Nau,Cc,YZl),(Nau,Cc,YZm),(Nau,Cc,YZn),(Nau,Cc,YZo) ,(Nau,Cc,YZp),(Nau,Cc,YZq),(Nau,Cc,YZr),(Nau,Cc,YZs),(Nau,Cc,YZt),(Nau,Cc,YZu),(N au,Cc,YZv),(Nau,Cc,YZw),(Nau,Cc,YZx),(Nau,Cc,YZy),(Nau,Cc,YZz),(Nau,Cc,YZaa),(Na u,Cc,YZab),(Nau,Cc,YZac),(Nau,Cc,YZad),(Nau,Cc,YZae),(Nau,Cc,YZaf),(Nau,Cc,YZag),( Nau,Cd,YZa),(Nau,Cd,YZb),(Nau,Cd,YZc),(Nau,Cd,YZd),(Nau,Cd,YZe),(Nau,Cd,YZf),(Na u,Cd,YZg),(Nau,Cd,YZh),(Nau,Cd,YZi),(Nau,Cd,YZj),(Nau,Cd,YZk),(Nau,Cd,YZl),(Nau,C d,YZm),(Nau,Cd,YZn),(Nau,Cd,YZo),(Nau,Cd,YZp),(Nau,Cd,YZq),(Nau,Cd,YZr),(Nau,Cd, YZs),(Nau,Cd,YZt),(Nau,Cd,YZu),(Nau,Cd,YZv),(Nau,Cd,YZw),(Nau,Cd,YZx),(Nau,Cd,Y Zy),(Nau,Cd,YZz),(Nau,Cd,YZaa),(Nau,Cd,YZab),(Nau,Cd,YZac),(Nau,Cd,YZad),(Nau,Cd, YZae),(Nau,Cd,YZaf),(Nau,Cd,YZag),(Nau,Ce,YZa),(Nau,Ce,YZb),(Nau,Ce,YZc),(Nau,Ce, YZd),(Nau,Ce,YZe),(Nau,Ce,YZf),(Nau,Ce,YZg),(Nau,Ce,YZh),(Nau,Ce,YZi),(Nau,Ce,YZj) ,(Nau,Ce,YZk),(Nau,Ce,YZl),(Nau,Ce,YZm),(Nau,Ce,YZn),(Nau,Ce,YZo),(Nau,Ce,YZp),( Nau,Ce,YZq),(Nau,Ce,YZr),(Nau,Ce,YZs),(Nau,Ce,YZt),(Nau,Ce,YZu),(Nau,Ce,YZv),(Nau, Ce,YZw),(Nau,Ce,YZx),(Nau,Ce,YZy),(Nau,Ce,YZz),(Nau,Ce,YZaa),(Nau,Ce,YZab),(Nau, Ce,YZac),(Nau,Ce,YZad),(Nau,Ce,YZae),(Nau,Ce,YZaf),(Nau,Ce,YZag),(Nau,Cf,YZa),(Na u,Cf,YZb),(Nau,Cf,YZc),(Nau,Cf,YZd),(Nau,Cf,YZe),(Nau,Cf,YZf),(Nau,Cf,YZg),(Nau,Cf, YZh),(Nau,Cf,YZi),(Nau,Cf,YZj),(Nau,Cf,YZk),(Nau,Cf,YZl),(Nau,Cf,YZm),(Nau,Cf,YZn), (Nau,Cf,YZo),(Nau,Cf,YZp),(Nau,Cf,YZq),(Nau,Cf,YZr),(Nau,Cf,YZs),(Nau,Cf,YZt),(Nau, Cf,YZu),(Nau,Cf,YZv),(Nau,Cf,YZw),(Nau,Cf,YZx),(Nau,Cf,YZy),(Nau,Cf,YZz),(Nau,Cf, YZaa),(Nau,Cf,YZab),(Nau,Cf,YZac),(Nau,Cf,YZad),(Nau,Cf,YZae),(Nau,Cf,YZaf),(Nau,Cf ,YZag),(Nau,Cg,YZa),(Nau,Cg,YZb),(Nau,Cg,YZc),(Nau,Cg,YZd),(Nau,Cg,YZe),(Nau,Cg, YZf),(Nau,Cg,YZg),(Nau,Cg,YZh),(Nau,Cg,YZi),(Nau,Cg,YZj),(Nau,Cg,YZk),(Nau,Cg,YZl ,(Nau,Cg,YZm),(Nau,Cg,YZn),(Nau,Cg,YZo),(Nau,Cg,YZp),(Nau,Cg,YZq),(Nau,Cg,YZr),( Nau,Cg,YZs),(Nau,Cg,YZt),(Nau,Cg,YZu),(Nau,Cg,YZv),(Nau,Cg,YZw),(Nau,Cg,YZx),(Na u,Cg,YZy),(Nau,Cg,YZz),(Nau,Cg,YZaa),(Nau,Cg,YZab),(Nau,Cg,YZac),(Nau,Cg,YZad),( Nau,Cg,YZae),(Nau,Cg,YZaf),(Nau,Cg,YZag),(Nau,Ch,YZa),(Nau,Ch,YZb),(Nau,Ch,YZc),( Nau,Ch,YZd),(Nau,Ch,YZe),(Nau,Ch,Ya),(Nau,Ch,YZg),(Nau,Ch,YZh),(Nau,Ch,YZi),(Na u,Ch,YZj),(Nau,Ch,YZl),(Nau,Ch,YZl),(Nau,Ch,YZm),(Nau,Ch,YZn),(Nau,Ch,YZo),(Nau, Ch,YZp),(Nau,Ch,YZq),(Nau,Ch,YZr),(Nau,Ch,YZs),(Nau,Ch,YZt),(Nau,Ch,YZu),(Nau,Ch, YZv),(Nau,Ch,YZw),(Nau,Ch,YZx),(Nau,Ch,YZy),(Nau,Ch,YZz),(Nau,Ch,YZaa),(Nau,Ch, YZab),(Nau,Ch,YZac),(Nau,Ch,YZad),(Nau,Ch,YZae),(Nau,Ch,YZaf),(Nau,Ch,YZag),(Nau, Ci,YZa),(Nau,Ci,YZb),(Nau,Ci,YZc),(Nau,Ci,YZd),(Nau,Ci,YZe),(Nau,Ci,YZf),(Nau,Ci,YZ g),(Nau,Ci,YZh),(Nau,Ci,YZi),(Nau,Ci,YZj),(Nau,Ci,YZk),(Nau,Ci,YZl),(Nau,Ci,YZm),(Na u,Ci,YZn),(Nau,Ci,YZo),(Nau,Ci,YZp),(Nau,Ci,YZq),(Nau,Ci,YZr),(Nau,Ci,YZs),(Nau,Ci,Y Zt),(Nau,Ci,YZu),(Nau,Ci,YZv),(Nau,Ci,YZw),(Nau,Ci,YZx),(Nau,Ci,YZy),(Nau,Ci,YZz),( Nau,Ci,YZaa),(Nau,Ci,YZab),(Nau,Ci,YZac),(Nau,Ci,YZad),(Nau,Ci,YZae),(Nau,Ci,YZaf),( Nau,Ci,YZag),(Nau,Cj,YZa),(Nau,Cj,YZb),(Nau,Cj,YZc),(Nau,Cj,YZd),(Nau,Cj,YZe),(Nau, Cj,Ya),(Nau,Cj,YZg),(Nau,Cj,YZh),(Nau,Cj,YZi),(Nau,Cj,YZj),(Nau,Cj,YZk),(Nau,Cj,YZl ,(Nau,Cj,YZm),(Nau,Cj,YZn),(Nau,Cj,YZo),(Nau,Cj,YZp),(Nau,Cj,YZq),(Nau,Cj,YZr),(Na u,Cj,YZs),(Nau,Cj,YZt),(Nau,Cj,YZu),(Nau,Cj,YZv),(Nau,Cj,YZw),(Nau,Cj,YZx),(Nau,Cj, YZy),(Nau,Cj,YZz),(Nau,Cj,YZaa),(Nau,Cj,YZab),(Nau,Cj,YZac),(Nau,Cj,YZad),(Nau,Cj,Y Zae),(Nau,Cj,YZaf),(Nau,Cj,YZag),(Nav,Ca,YZa),(Nav,Ca,YZb),(Nav,Ca,YZc),(Nav,Ca,YZ d),(Nav,Ca,YZe),(Nav,Ca,YZf),(Nav,Ca,YZg),(Nav,Ca,YZh),(Nav,Ca,YZi),(Nav,Ca,YZj),( Nav,Ca,YZk),(Nav,Ca,YZl),(Nav,Ca,YZm),(Nav,Ca,YZn),(Nav,Ca,YZo),(Nav,Ca,YZp),(Na v,Ca,YZq),(Nav,Ca,YZr),(Nav,Ca,YZs),(Nav,Ca,YZt),(Nav,Ca,YZu),(Nav,Ca,YZv),(Nav,Ca ,YZw),(Nav,Ca,YZx),(Nav,Ca,YZy),(Nav,Ca,YZz),(Nav,Ca,YZaa),(Nav,Ca,YZab),(Nav,Ca, YZac),(Nav,Ca,YZad),(Nav,Ca,YZae),(Nav,Ca,YZaf),(Nav,Ca,YZag),(Nav,Cb,YZa),(Nav,C b,YZb),(Nav,Cb,YZc),(Nav,Cb,YZd),(Nav,Cb,YZe),(Nav,Cb,YZf),(Nav,Cb,YZg),(Nav,Cb, YZh),(Nav,Cb,YZi),(Nav,Cb,YZj),(Nav,Cb,YZk),(Nav,Cb,YZl),(Nav,Cb,YZm),(Nav,Cb,YZ n),(Nav,Cb,YZo),(Nav,Cb,YZp),(Nav,Cb,YZq),(Nav,Cb,YZr),(Nav,Cb,YZs),(Nav,Cb,YZt),( Nav,Cb,YZu),(Nav,Cb,YZv),(Nav,Cb,YZw),(Nav,Cb,YZx),(Nav,Cb,YZy),(Nav,Cb,YZz),(N av,Cb,YZaa),(Nav,Cb,YZab),(Nav,Cb,YZac),(Nav,Cb,YZad),(Nav,Cb,YZae),(Nav,Cb,YZaf) ,(Nav,Cb,YZag),(Nav,Cc,YZa),(Nav,Cc,YZb),(Nav,Cc,YZc),(Nav,Cc,YZd),(Nav,Cc,YZe),( Nav,Cc,YZf),(Nav,Cc,YZg),(Nav,Cc,YZh),(Nav,Cc,YZi),(Nav,Cc,YZj),(Nav,Cc,YZk),(Nav, Cc,YZl),(Nav,Cc,YZm),(Nav,Cc,YZn),(Nav,Cc,YZo),(Nav,Cc,YZp),(Nav,Cc,YZq),(Nav,Cc, YZr),(Nav,Cc,YZs),(Nav,Cc,YZt),(Nav,Cc,YZu),(Nav,Cc,YZv),(Nav,Cc,YZw),(Nav,Cc,YZ x),(Nav,Cc,YZy),(Nav,Cc,YZz),(Nav,Cc,YZaa),(Nav,Cc,YZab),(Nav,Cc,YZac),(Nav,Cc,YZ ad),(Nav,Cc,YZae),(Nav,Cc,YZaf),(Nav,Cc,YZag),(Nav,Cd,YZa),(Nav,Cd,YZb),(Nav,Cd,Y Zc),(Nav,Cd,YZd),(Nav,Cd,YZe),(Nav,Cd,YZf),(Nav,Cd,YZg),(Nav,Cd,YZh),(Nav,Cd,YZi) ,(Nav,Cd,YZj),(Nav,Cd,YZk),(Nav,Cd,YZl),(Nav,Cd,YZm),(Nav,Cd,YZn),(Nav,Cd,YZo),( Nav,Cd,YZp),(Nav,Cd,YZq),(Nav,Cd,YZr),(Nav,Cd,YZs),(Nav,Cd,YZt),(Nav,Cd,YZu),(Na v,Cd,YZv),(Nav,Cd,YZw),(Nav,Cd,YZx),(Nav,Cd,YZy),(Nav,Cd,YZz),(Nav,Cd,YZaa),(Nav ,Cd,YZab),(Nav,Cd,YZac),(Nav,Cd,YZad),(Nav,Cd,YZae),(Nav,Cd,YZaf),(Nav,Cd,YZag),( Nav,Ce,YZa),(Nav,Ce,YZb),(Nav,Ce,YZc),(Nav,Ce,YZd),(Nav,Ce,YZe),(Nav,Ce,YZf),(Nav ,Ce,YZg),(Nav,Ce,YZh),(Nav,Ce,YZi),(Nav,Ce,YZj),(Nav,Ce,YZk),(Nav,Ce,YZl),(Nav,Ce, YZm),(Nav,Ce,YZn),(Nav,Ce,YZo),(Nav,Ce,YZp),(Nav,Ce,YZq),(Nav,Ce,YZr),(Nav,Ce,YZ s),(Nav,Ce,YZt),(Nav,Ce,YZu),(Nav,Ce,YZv),(Nav,Ce,YZw),(Nav,Ce,YZx),(Nav,Ce,YZy),( Nav,Ce,YZz),(Nav,Ce,YZaa),(Nav,Ce,YZab),(Nav,Ce,YZac),(Nav,Ce,YZad),(Nav,Ce,YZae) ,(Nav,Ce,YZaf),(Nav,Ce,YZag),(Nav,Cf,YZa),(Nav,Cf,YZb),(Nav,Cf,YZc),(Nav,Cf,YZd),( Nav,Cf,YZe),(Nav,Cf,YZf),(Nav,Cf,YZg),(Nav,Cf,YZh),(Nav,Cf,YZi),(Nav,Cf,YZj),(Nav,C f,YZk),(Nav,Cf,YZl),(Nav,Cf,YZm),(Nav,Cf,YZn),(Nav,Cf,YZo),(Nav,Cf,YZp),(Nav,Cf,YZ q),(Nav,Cf,YZr),(Nav,Cf,YZs),(Nav,Cf,YZt),(Nav,Cf,YZu),(Nav,Cf,YZv),(Nav,Cf,YZw),(N av,Cf,YZx),(Nav,Cf,YZy),(Nav,Cf,YZz),(Nav,Cf,YZaa),(Nav,Cf,YZab),(Nav,Cf,YZac),(Nav ,Cf,YZad),(Nav,Cf,YZae),(Nav,Cf,YZaf),(Nav,Cf,YZag),(Nav,Cg,YZa),(Nav,Cg,YZb),(Nav, Cg,YZc),(Nav,Cg,YZd),(Nav,Cg,YZe),(Nav,Cg,YZf),(Nav,Cg,YZg),(Nav,Cg,YZh),(Nav,Cg ,YZi),(Nav,Cg,YZj),(Nav,Cg,YZk),(Nav,Cg,YZl),(Nav,Cg,YZm),(Nav,Cg,YZn),(Nav,Cg,Y Zo),(Nav,Cg,YZp),(Nav,Cg,YZq),(Nav,Cg,YZr),(Nav,Cg,YZs),(Nav,Cg,YZt),(Nav,Cg,YZu) ,(Nav,Cg,YZv),(Nav,Cg,YZw),(Nav,Cg,YZx),(Nav,Cg,YZy),(Nav,Cg,YZz),(Nav,Cg,YZaa), (Nav,Cg,YZab),(Nav,Cg,YZac),(Nav,Cg,YZad),(Nav,Cg,YZae),(Nav,Cg,YZaf),(Nav,Cg,YZ ag),(Nav,Ch,YZa),(Nav,Ch,YZb),(Nav,Ch,YZc),(Nav,Ch,YZd),(Nav,Ch,YZe),(Nav,Ch,YZf) ,(Nav,Ch,YZg),(Nav,Ch,YZh),(Nav,Ch,YZi),(Nav,Ch,YZj),(Nav,Ch,YZk),(Nav,Ch,YZl),(Na v,Ch,YZm),(Nav,Ch,YZn),(Nav,Ch,YZo),(Nav,Ch,YZp),(Nav,Ch,YZq),(Nav,Ch,YZr),(Nav, Ch,YZs),(Nav,Ch,YZt),(Nav,Ch,YZu),(Nav,Ch,YZv),(Nav,Ch,YZw),(Nav,Ch,YZx),(Nav,Ch ,YZy),(Nav,Ch,YZz),(Nav,Ch,YZaa),(Nav,Ch,YZab),(Nav,Ch,YZac),(Nav,Ch,YZad),(Nav,C h,YZae),(Nav,Ch,YZaf),(Nav,Ch,YZag),(Nav,Ci,YZa),(Nav,Ci,YZb),(Nav,Ci,YZc),(Nav,Ci, YZd),(Nav,Ci,YZe),(Nav,Ci,YZf),(Nav,Ci,YZg),(Nav,Ci,YZh),(Nav,Ci,YZi),(Nav,Ci,YZj),( Nav,Ci,YZk),(Nav,Ci,YZl),(Nav,Ci,YZm),(Nav,Ci,YZn),(Nav,Ci,YZo),(Nav,Ci,YZp),(Nav, Ci,YZq),(Nav,Ci,YZr),(Nav,Ci,YZs),(Nav,Ci,YZt),(Nav,Ci,YZu),(Nav,Ci,YZv),(Nav,Ci,YZ w),(Nav,Ci,YZx),(Nav,Ci,YZy),(Nav,Ci,YZz),(Nav,Ci,YZaa),(Nav,Ci,YZab),(Nav,Ci,YZac) ,(Nav,Ci,YZad),(Nav,Ci,YZae),(Nav,Ci,YZaf),(Nav,Ci,YZag),(Nav,Cj,YZa),(Nav,Cj,YZb),( Nav,Cj,YZc),(Nav,Cj,YZd),(Nav,Cj,YZe),(Nav,Cj,YZf),(Nav,Cj,YZg),(Nav,Cj,YZh),(Nav,C j,YZi),(Nav,Cj,YZj),(Nav,Cj,YZk),(Nav,Cj,YZl),(Nav,Cj,YZm),(Nav,Cj,YZn),(Nav,Cj,YZo) ,(Nav,Cj,YZp),(Nav,Cj,YZq),(Nav,Cj,YZr),(Nav,Cj,YZs),(Nav,Cj,YZt),(Nav,Cj,YZu),(Nav, Cj,YZv),(Nav,Cj,YZw),(Nav,Cj,YZx),(Nav,Cj,YZy),(Nav,Cj,YZz),(Nav,Cj,YZaa),(Nav,Cj, YZab),(Nav,Cj,YZac),(Nav,Cj,YZad),(Nav,Cj,YZae),(Nav,Cj,YZaf),(Nav,Cj,YZag),(Naw,C a,YZa),(Naw,Ca,YZb),(Naw,Ca,YZc),(Naw,Ca,YZd),(Naw,Ca,YZe),(Naw,Ca,YZf),(Naw,Ca ,YZg),(Naw,Ca,YZh),(Naw,Ca,YZi),(Naw,Ca,YZj),(Naw,Ca,YZk),(Naw,Ca,YZl),(Naw,Ca, YZm),(Naw,Ca,YZn),(Naw,Ca,YZo),(Naw,Ca,YZp),(Naw,Ca,YZq),(Naw,Ca,YZr),(Naw,Ca, YZs),(Naw,Ca,YZt),(Naw,Ca,YZu),(Naw,Ca,YZv),(Naw,Ca,YZw),(Naw,Ca,YZx),(Naw,Ca, YZy),(Naw,Ca,YZz),(Naw,Ca,YZaa),(Naw,Ca,YZab),(Naw,Ca,YZac),(Naw,Ca,YZad),(Naw ,Ca,YZae),(Naw,Ca,YZaf),(Naw,Ca,YZag),(Naw,Cb,YZa),(Naw,Cb,YZb),(Naw,Cb,YZc),(N aw,Cb,YZd),(Naw,Cb,YZe),(Naw,Cb,YZf),(Naw,Cb,YZg),(Naw,Cb,YZh),(Naw,Cb,YZi),(N aw,Cb,YZj),(Naw,Cb,YZk),(Naw,Cb,YZl),(Naw,Cb,YZm),(Naw,Cb,YZn),(Naw,Cb,YZo),( Naw,Cb,YZp),(Naw,Cb,YZq),(Naw,Cb,YZr),(Naw,Cb,YZs),(Naw,Cb,YZt),(Naw,Cb,YZu),( Naw,Cb,YZv),(Naw,Cb,YZw),(Naw,Cb,YZx),(Naw,Cb,YZy),(Naw,Cb,YZz),(Naw,Cb,YZaa) ,(Naw,Cb,YZab),(Naw,Cb,YZac),(Naw,Cb,YZad),(Naw,Cb,YZae),(Naw,Cb,YZaf),(Naw,C b,YZag),(Naw,Cc,YZa),(Naw,Cc,YZb),(Naw,Cc,YZc),(Naw,Cc,YZd),(Naw,Cc,YZe),(Naw, Cc,YZf),(Naw,Cc,YZg),(Naw,Cc,YZh),(Naw,Cc,YZi),(Naw,Cc,YZj),(Naw,Cc,YZk),(Naw,C c,YZl),(Naw,Cc,YZm),(Naw,Cc,YZn),(Naw,Cc,YZo),(Naw,Cc,YZp),(Naw,Cc,YZq),(Naw,C c,YZr),(Naw,Cc,YZs),(Naw,Cc,YZt),(Naw,Cc,YZu),(Naw,Cc,YZv),(Naw,Cc,YZw),(Naw,C c,YZx),(Naw,Cc,YZy),(Naw,Cc,YZz),(Naw,Cc,YZaa),(Naw,Cc,YZab),(Naw,Cc,YZac),(Na w,Cc,YZad),(Naw,Cc,YZae),(Naw,Cc,YZaf),(Naw,Cc,YZag),(Naw,Cd,YZa),(Naw,Cd,YZb), (Naw,Cd,YZc),(Naw,Cd,YZd),(Naw,Cd,YZe),(Naw,Cd,YZf),(Naw,Cd,YZg),(Naw,Cd,YZh), (Naw,Cd,YZi),(Naw,Cd,YZj),(Naw,Cd,YZk),(Naw,Cd,YZl),(Naw,Cd,YZm),(Naw,Cd,YZn), (Naw,Cd,YZo),(Naw,Cd,YZp),(Naw,Cd,YZq),(Naw,Cd,YZr),(Naw,Cd,YZs),(Naw,Cd,YZt), (Naw,Cd,YZu),(Naw,Cd,YZv),(Naw,Cd,YZw),(Naw,Cd,YZx),(Naw,Cd,YZy),(Naw,Cd,YZz) ,(Naw,Cd,YZaa),(Naw,Cd,YZab),(Naw,Cd,YZac),(Naw,Cd,YZad),(Naw,Cd,YZae),(Naw,C d,YZaf),(Naw,Cd,YZag),(Naw,Ce,YZa),(Naw,Ce,YZb),(Naw,Ce,YZc),(Naw,Ce,YZd),(Naw, Ce,YZe),(Naw,Ce,YZf),(Naw,Ce,YZg),(Naw,Ce,YZh),(Naw,Ce,YZi),(Naw,Ce,YZj),(Naw,C e,YZk),(Naw,Ce,YZl),(Naw,Ce,YZm),(Naw,Ce,YZn),(Naw,Ce,YZo),(Naw,Ce,YZp),(Naw,C e,YZq),(Naw,Ce,YZr),(Naw,Ce,YZs),(Naw,Ce,YZt),(Naw,Ce,YZu),(Naw,Ce,YZv),(Naw,Ce ,YZw),(Naw,Ce,YZx),(Naw,Ce,YZy),(Naw,Ce,YZz),(Naw,Ce,YZaa),(Naw,Ce,YZab),(Naw, Ce,YZac),(Naw,Ce,YZad),(Naw,Ce,YZae),(Naw,Ce,YZaf),(Naw,Ce,YZag),(Naw,Cf,YZa),( Naw,Cf,YZb),(Naw,Cf,YZc),(Naw,Cf,YZd),(Naw,Cf,YZe),(Naw,Cf,YZf),(Naw,Cf,YZg),(N aw,Cf,YZh),(Naw,Cf,YZi),(Naw,Cf,YZj),(Naw,Cf,YZk),(Naw,Cf,YZl),(Naw,Cf,YZm),(Naw ,Cf,YZn),(Naw,Cf,YZo),(Naw,Cf,YZp),(Naw,Cf,YZq),(Naw,Cf,YZr),(Naw,Cf,YZs),(Naw,C f,YZt),(Naw,Cf,YZu),(Naw,Cf,YZv),(Naw,Cf,YZw),(Naw,Cf,YZx),(Naw,Cf,YZy),(Naw,Cf, YZz),(Naw,Cf,YZaa),(Naw,Cf,YZab),(Naw,Cf,YZac),(Naw,Cf,YZad),(Naw,Cf,YZae),(Naw, Cf,YZaf),(Naw,Cf,YZag),(Naw,Cg,YZa),(Naw,Cg,YZb),(Naw,Cg,YZc),(Naw,Cg,YZd),(Na w,Cg,YZe),(Naw,Cg,YZf),(Naw,Cg,YZg),(Naw,Cg,YZh),(Naw,Cg,YZi),(Naw,Cg,YZj),(Na w,Cg,YZk),(Naw,Cg,YZl),(Naw,Cg,YZm),(Naw,Cg,YZn),(Naw,Cg,YZo),(Naw,Cg,YZp),(N aw,Cg,YZq),(Naw,Cg,YZr),(Naw,Cg,YZs),(Naw,Cg,YZt),(Naw,Cg,YZu),(Naw,Cg,YZv),(N aw,Cg,YZw),(Naw,Cg,YZx),(Naw,Cg,YZy),(Naw,Cg,YZz),(Naw,Cg,YZaa),(Naw,Cg,YZab) ,(Naw,Cg,YZac),(Naw,Cg,YZad),(Naw,Cg,YZae),(Naw,Cg,YZaf),(Naw,Cg,YZag),(Naw,Ch, YZa),(Naw,Ch,YZb),(Naw,Ch,YZc),(Naw,Ch,YZd),(Naw,Ch,YZe),(Naw,Ch,YZf),(Naw,Ch, YZg),(Naw,Ch,YZh),(Naw,Ch,YZi),(Naw,Ch,YZj),(Naw,Ch,YZk),(Naw,Ch,YZl),(Naw,Ch, YZm),(Naw,Ch,YZn),(Naw,Ch,YZo),(Naw,Ch,YZp),(Naw,Ch,YZq),(Naw,Ch,YZr),(Naw,C h,YZs),(Naw,Ch,YZt),(Naw,Ch,YZu),(Naw,Ch,YZv),(Naw,Ch,YZw),(Naw,Ch,YZx),(Naw, Ch,YZy),(Naw,Ch,YZz),(Naw,Ch,YZaa),(Naw,Ch,YZab),(Naw,Ch,YZac),(Naw,Ch,YZad),( Naw,Ch,YZae),(Naw,Ch,YZaf),(Naw,Ch,YZag),(Naw,Ci,YZa),(Naw,Ci,YZb),(Naw,Ci,YZc) ,(Naw,Ci,YZd),(Naw,Ci,YZe),(Naw,Ci,YZf),(Naw,Ci,YZg),(Naw,Ci,YZh),(Naw,Ci,YZi),(N aw,Ci,YZj),(Naw,Ci,YZk),(Naw,Ci,YZl),(Naw,Ci,YZm),(Naw,Ci,YZn),(Naw,Ci,YZo),(Naw ,Ci,YZp),(Naw,Ci,YZq),(Naw,Ci,YZr),(Naw,Ci,YZs),(Naw,Ci,YZt),(Naw,Ci,YZu),(Naw,Ci, YZv),(Naw,Ci,YZw),(Naw,Ci,YZx),(Naw,Ci,YZy),(Naw,Ci,YZz),(Naw,Ci,YZaa),(Naw,Ci, YZab),(Naw,Ci,YZac),(Naw,Ci,YZad),(Naw,Ci,YZae),(Naw,Ci,YZaf),(Naw,Ci,YZag),(Naw ,Cj,YZa),(Naw,Cj,YZb),(Naw,Cj,YZc),(Naw,Cj,YZd),(Naw,Cj,YZe),(Naw,Cj,YZf),(Naw,Cj, YZg),(Naw,Cj,YZh),(Naw,Cj,YZi),(Naw,Cj,YZj),(Naw,Cj,YZk),(Naw,Cj,YZl),(Naw,Cj,YZ m),(Naw,Cj,YZn),(Naw,Cj,YZo),(Naw,Cj,YZp),(Naw,Cj,YZq),(Naw,Cj,YZr),(Naw,Cj,YZs), (Naw,Cj,YZt),(Naw,Cj,YZu),(Naw,Cj,YZv),(Naw,Cj,YZw),(Naw,Cj,YZx),(Naw,Cj,YZy),(N aw,Cj,YZz),(Naw,Cj,YZaa),(Naw,Cj,YZab),(Naw,Cj,YZac),(Naw,Cj,YZad),(Naw,q,YZae), (Naw,Cj,YZaf),(Naw,Cj,YZag),(Nax,Ca,YZa),(Nax,Ca,YZb),(Nax,Ca,YZc),(Nax,Ca,YZd),( Nax,Ca,YZe),(Nax,Ca,YZf),(Nax,Ca,YZg),(Nax,Ca,YZh),(Nax,Ca,YZi),(Nax,Ca,YZj),(Nax, Ca,YZk),(Nax,Ca,YZl),(Nax,Ca,YZm),(Nax,Ca,YZn),(Nax,Ca,YZo),(Nax,Ca,YZp),(Nax,Ca, YZq),(Nax,Ca,YZr),(Nax,Ca,YZs),(Nax,Ca,YZt),(Nax,Ca,YZu),(Nax,Ca,YZv),(Nax,Ca,YZ w),(Nax,Ca,YZx),(Nax,Ca,YZy),(Nax,Ca,YZz),(Nax,Ca,YZaa),(Nax,Ca,YZab),(Nax,Ca,YZa c),(Nax,Ca,YZad),(Nax,Ca,YZae),(Nax,Ca,YZaf),(Nax,Ca,YZag),(Nax,Cb,YZa),(Nax,Cb,Y Zb),(Nax,Cb,YZc),(Nax,Cb,YZd),(Nax,Cb,YZe),(Nax,Cb,YZf),(Nax,Cb,YZg),(Nax,Cb,YZh) ,(Nax,Cb,YZi),(Nax,Cb,YZj),(Nax,Cb,YZk),(Nax,Cb,YZl),(Nax,Cb,YZm),(Nax,Cb,YZn),( Nax,Cb,YZo),(Nax,Cb,YZp),(Nax,Cb,YZq),(Nax,Cb,YZr),(Nax,Cb,YZs),(Nax,Cb,YZt),(Na x,Cb,YZu),(Nax,Cb,YZv),(Nax,Cb,YZw),(Nax,Cb,YZx),(Nax,Cb,YZy),(Nax,Cb,YZz),(Nax, Cb,YZaa),(Nax,Cb,YZab),(Nax,Cb,YZac),(Nax,Cb,YZad),(Nax,Cb,YZae),(Nax,Cb,YZaf),( Nax,Cb,YZag),(Nax,Cc,YZa),(Nax,Cc,YZb),(Nax,Cc,YZc),(Nax,Cc,YZd),(Nax,Cc,YZe),(N ax,Cc,YZf),(Nax,Cc,YZg),(Nax,Cc,YZh),(Nax,Cc,YZi),(Nax,Cc,YZj),(Nax,Cc,YZk),(Nax,C c,YZl),(Nax,Cc,YZm),(Nax,Cc,YZn),(Nax,Cc,YZo),(Nax,Cc,YZp),(Nax,Cc,YZq),(Nax,Cc, YZr),(Nax,Cc,YZs),(Nax,Cc,YZt),(Nax,Cc,YZu),(Nax,Cc,YZv),(Nax,Cc,YZw),(Nax,Cc,YZ x),(Nax,Cc,YZy),(Nax,Cc,YZz),(Nax,Cc,YZaa),(Nax,Cc,YZab),(Nax,Cc,YZac),(Nax,Cc,YZ ad),(Nax,Cc,YZae),(Nax,Cc,YZaf),(Nax,Cc,YZag),(Nax,Cd,YZa),(Nax,Cd,YZb),(Nax,Cd,Y Zc),(Nax,Cd,YZd),(Nax,Cd,YZe),(Nax,Cd,YZf),(Nax,Cd,YZg),(Nax,Cd,YZh),(Nax,Cd,YZi) ,(Nax,Cd,YZj),(Nax,Cd,YZk),(Nax,Cd,YZl),(Nax,Cd,YZm),(Nax,Cd,YZn),(Nax,Cd,YZo),( Nax,Cd,YZp),(Nax,Cd,YZq),(Nax,Cd,YZr),(Nax,Cd,YZs),(Nax,Cd,YZt),(Nax,Cd,YZu),(Na x,Cd,YZv),(Nax,Cd,YZw),(Nax,Cd,YZx),(Nax,Cd,YZy),(Nax,Cd,YZz),(Nax,Cd,YZaa),(Nax ,Cd,YZab),(Nax,Cd,YZac),(Nax,Cd,YZad),(Nax,Cd,YZae),(Nax,Cd,YZaf),(Nax,Cd,YZag),( Nax,Ce,YZa),(Nax,Ce,YZb),(Nax,Ce,YZc),(Nax,Ce,YZd),(Nax,Ce,YZe),(Nax,Ce,YZf),(Nax ,Ce,YZg),(Nax,Ce,YZh),(Nax,Ce,YZi),(Nax,Ce,YZj),(Nax,Ce,YZk),(Nax,Ce,YZl),(Nax,Ce, YZm),(Nax,Ce,YZn),(Nax,Ce,YZo),(Nax,Ce,YZp),(Nax,Ce,YZq),(Nax,Ce,YZr),(Nax,Ce,YZ s),(Nax,Ce,YZt),(Nax,Ce,YZu),(Nax,Ce,YZv),(Nax,Ce,YZw),(Nax,Ce,YZx),(Nax,Ce,YZy),( Nax,Ce,YZz),(Nax,Ce,YZaa),(Nax,Ce,YZab),(Nax,Ce,YZac),(Nax,Ce,YZad),(Nax,Ce,YZae) ,(Nax,Ce,YZaf),(Nax,Ce,YZag),(Nax,Cf,YZa),(Nax,Cf,YZb),(Nax,Cf,YZc),(Nax,Cf,YZd),( Nax,Cf,YZe),(Nax,Cf,Ya),(Nax,Cf,YZg),(Nax,Cf,YZh),(Nax,Cf,YZi),(Nax,Cf,YZj),(Nax,C f,YZk),(Nax,Cf,YZl),(Nax,Cf,YZm),(Nax,Cf,YZn),(Nax,Cf,YZo),(Nax,Cf,YZp),(Nax,Cf,YZ q),(Nax,Cf,YZr),(Nax,Cf,YZs),(Nax,Cf,YZt),(Nax,Cf,YZu),(Nax,Cf,YZv),(Nax,Cf,YZw),(N ax,Cf,YZx),(Nax,Cf,YZy),(Nax,Cf,YZz),(Nax,Cf,YZaa),(Nax,Cf,YZab),(Nax,Cf,YZac),(Nax ,Cf,YZad),(Nax,Cf,YZae),(Nax,Cf,YZaf),(Nax,Cf,YZag),(Nax,Cg,YZa),(Nax,Cg,YZb),(Nax, Cg,YZc),(Nax,Cg,YZd),(Nax,Cg,YZe),(Nax,Cg,YZf),(Nax,Cg,YZg),(Nax,Cg,YZh),(Nax,Cg ,YZi),(Nax,Cg,YZj),(Nax,Cg,YZk),(Nax,Cg,YZl),(Nax,Cg,YZm),(Nax,Cg,YZn),(Nax,Cg,Y Zo),(Nax,Cg,YZp),(Nax,Cg,YZq),(Nax,Cg,YZr),(Nax,Cg,YZs),(Nax,Cg,YZt),(Nax,Cg,YZu) ,(Nax,Cg,YZv),(Nax,Cg,YZw),(Nax,Cg,YZx),(Nax,Cg,YZy),(Nax,Cg,YZz),(Nax,Cg,YZaa), (Nax,Cg,YZab),(Nax,Cg,YZac),(Nax,Cg,YZad),(Nax,Cg,YZae),(Nax,Cg,YZaf),(Nax,Cg,YZ ag),(Nax,Ch,YZa),(Nax,Ch,YZb),(Nax,Ch,YZc),(Nax,Ch,YZd),(Nax,Ch,YZe),(Nax,Ch,YZf) ,(Nax,Ch,YZg),(Nax,Ch,YZh),(Nax,Ch,YZi),(Nax,Ch,YZj),(Nax,Ch,YZk),(Nax,Ch,YZl),(Na x,Ch,YZm),(Nax,Ch,YZn),(Nax,Ch,YZo),(Nax,Ch,YZp),(Nax,Ch,YZq),(Nax,Ch,YZr),(Nax, Ch,YZs),(Nax,Ch,YZt),(Nax,Ch,YZu),(Nax,Ch,YZv),(Nax,Ch,YZw),(Nax,Ch,YZx),(Nax,Ch ,YZy),(Nax,Ch,YZz),(Nax,Ch,YZaa),(Nax,Ch,YZab),(Nax,Ch,YZac),(Nax,Ch,YZad),(Nax,C h,YZae),(Nax,Ch,YZaf),(Nax,Ch,YZag),(Nax,Ci,YZa),(Nax,Ci,YZb),(Nax,Ci,YZc),(Nax,Ci, YZd),(Nax,Ci,YZe),(Nax,Ci,YZf),(Nax,Ci,YZg),(Nax,Ci,YZh),(Nax,Ci,YZi),(Nax,Ci,YZj),( Nax,Ci,YZk),(Nax,Ci,YZl),(Nax,Ci,YZm),(Nax,Ci,YZn),(Nax,Ci,YZo),(Nax,Ci,YZp),(Nax, Ci,YZq),(Nax,Ci,YZr),(Nax,Ci,YZs),(Nax,Ci,YZt),(Nax,Ci,YZu),(Nax,Ci,YZv),(Nax,Ci,YZ w),(Nax,Ci,YZx),(Nax,Ci,YZy),(Nax,Ci,YZz),(Nax,Ci,YZaa),(Nax,Ci,YZab),(Nax,Ci,YZac) ,(Nax,Ci,YZad),(Nax,Ci,YZae),(Nax,Ci,YZaf),(Nax,Ci,YZag),(Nax,Cj,YZa),(Nax,Cj,YZb),( Nax,Cj,YZc),(Nax,Cj,YZd),(Nax,Cj,YZe),(Nax,Cj,YZf),(Nax,Cj,YZg),(Nax,Cj,YZh),(Nax,C j,YZi),(Nax,Cj,YZj),(Nax,Cj,YZk),(Nax,Cj,YZl),(Nax,Cj,YZm),(Nax,Cj,YZn),(Nax,Cj,YZo) ,(Nax,Cj,YZp),(Nax,Cj,YZq),(Nax,Cj,YZr),(Nax,Cj,YZs),(Nax,Cj,YZt),(Nax,Cj,YZu),(Nax, Cj,YZv),(Nax,Cj,YZw),(Nax,Cj,YZx),(Nax,Cj,YZy),(Nax,Cj,YZz),(Nax,Cj,YZaa),(Nax,Cj, YZab),(Nax,Cj,YZac),(Nax,Cj,YZad),(Nax,Cj,YZae),(Nax,Cj,YZaf),(Nax,Cj,YZag),(Nay,Ca ,YZa),(Nay,Ca,YZb),(Nay,Ca,YZc),(Nay,Ca,YZd),(Nay,Ca,YZe),(Nay,Ca,YZf),(Nay,Ca,YZ g),(Nay,Ca,YZh),(Nay,Ca,YZi),(Nay,Ca,YZj),(Nay,Ca,YZk),(Nay,Ca,YZl),(Nay,Ca,YZm),( Nay,Ca,YZn),(Nay,Ca,YZo),(Nay,Ca,YZp),(Nay,Ca,YZq),(Nay,Ca,YZr),(Nay,Ca,YZs),(Nay ,Ca,YZt),(Nay,Ca,YZu),(Nay,Ca,YZv),(Nay,Ca,YZw),(Nay,Ca,YZx),(Nay,Ca,YZy),(Nay,Ca ,YZz),(Nay,Ca,YZaa),(Nay,Ca,YZab),(Nay,Ca,YZac),(Nay,Ca,YZad),(Nay,Ca,YZae),(Nay, Ca,YZaf),(Nay,Ca,YZag),(Nay,Cb,YZa),(Nay,Cb,YZb),(Nay,Cb,YZc),(Nay,Cb,YZd),(Nay, Cb,YZe),(Nay,Cb,YZf),(Nay,Cb,YZg),(Nay,Cb,YZh),(Nay,Cb,YZi),(Nay,Cb,YZj),(Nay,Cb, YZk),(Nay,Cb,YZl),(Nay,Cb,YZm),(Nay,Cb,YZn),(Nay,Cb,YZo),(Nay,Cb,YZp),(Nay,Cb,Y Zq),(Nay,Cb,YZr),(Nay,Cb,YZs),(Nay,Cb,YZt),(Nay,Cb,YZu),(Nay,Cb,YZv),(Nay,Cb,YZw) ,(Nay,Cb,YZx),(Nay,Cb,YZy),(Nay,Cb,YZz),(Nay,Cb,YZaa),(Nay,Cb,YZab),(Nay,Cb,YZa c),(Nay,Cb,YZad),(Nay,Cb,YZae),(Nay,Cb,YZaf),(Nay,Cb,YZag),(Nay,Cc,YZa),(Nay,Cc,Y Zb),(Nay,Cc,YZc),(Nay,Cc,YZd),(Nay,Cc,YZe),(Nay,Cc,YZf),(Nay,Cc,YZg),(Nay,Cc,YZh), (Nay,Cc,YZi),(Nay,Cc,YZj),(Nay,Cc,YZk),(Nay,Cc,YZl),(Nay,Cc,YZm),(Nay,Cc,YZn),(Na y,Cc,YZo),(Nay,Cc,YZp),(Nay,Cc,YZq),(Nay,Cc,YZr),(Nay,Cc,YZs),(Nay,Cc,YZt),(Nay,Cc ,YZu),(Nay,Cc,YZv),(Nay,Cc,YZw),(Nay,Cc,YZx),(Nay,Cc,YZy),(Nay,Cc,YZz),(Nay,Cc,Y Zaa),(Nay,Cc,YZab),(Nay,Cc,YZac),(Nay,Cc,YZad),(Nay,Cc,YZae),(Nay,Cc,YZaf),(Nay,Cc ,YZag),(Nay,Cd,YZa),(Nay,Cd,YZb),(Nay,Cd,YZc),(Nay,Cd,YZd),(Nay,Cd,YZe),(Nay,Cd, YZf),(Nay,Cd,YZg),(Nay,Cd,YZh),(Nay,Cd,YZi),(Nay,Cd,YZj),(Nay,Cd,YZk),(Nay,Cd,YZl) ,(Nay,Cd,YZm),(Nay,Cd,YZn),(Nay,Cd,YZo),(Nay,Cd,YZp),(Nay,Cd,YZq),(Nay,Cd,YZr),( Nay,Cd,YZs),(Nay,Cd,YZt),(Nay,Cd,YZu),(Nay,Cd,YZv),(Nay,Cd,YZw),(Nay,Cd,YZx),(Na y,Cd,YZy),(Nay,Cd,YZz),(Nay,Cd,YZaa),(Nay,Cd,YZab),(Nay,Cd,YZac),(Nay,Cd,YZad),( Nay,Cd,YZae),(Nay,Cd,YZaf),(Nay,Cd,YZag),(Nay,Ce,YZa),(Nay,Ce,YZb),(Nay,Ce,YZc),( Nay,Ce,YZd),(Nay,Ce,YZe),(Nay,Ce,YZf),(Nay,Ce,YZg),(Nay,Ce,YZh),(Nay,Ce,YZi),(Nay ,Ce,YZj),(Nay,Ce,YZk),(Nay,Ce,YZl),(Nay,Ce,YZm),(Nay,Ce,YZn),(Nay,Ce,YZo),(Nay,Ce, YZp),(Nay,Ce,YZq),(Nay,Ce,YZr),(Nay,Ce,YZs),(Nay,Ce,YZt),(Nay,Ce,YZu),(Nay,Ce,YZv ),(Nay,Ce,YZw),(Nay,Ce,YZx),(Nay,Ce,YZy),(Nay,Ce,YZz),(Nay,Ce,YZaa),(Nay,Ce,YZab) ,(Nay,Ce,YZac),(Nay,Ce,YZad),(Nay,Ce,YZae),(Nay,Ce,YZaf),(Nay,Ce,YZag),(Nay,Cf,YZa) ,(Nay,Cf,YZb),(Nay,Cf,YZc),(Nay,Cf,YZd),(Nay,Cf,YZe),(Nay,Cf,YZf),(Nay,Cf,YZg),(Na y,Cf,YZh),(Nay,Cf,YZi),(Nay,Cf,YZj),(Nay,Cf,YZk),(Nay,Cf,YZl),(Nay,Cf,YZm),(Nay,Cf, YZn),(Nay,Cf,YZo),(Nay,Cf,YZp),(Nay,Cf,YZq),(Nay,Cf,YZr),(Nay,Cf,YZs),(Nay,Cf,YZt), (Nay,Cf,YZu),(Nay,Cf,YZv),(Nay,Cf,YZw),(Nay,Cf,YZx),(Nay,Cf,YZy),(Nay,Cf,YZz),(Na y,Cf,YZaa),(Nay,Cf,YZab),(Nay,Cf,YZac),(Nay,Cf,YZad),(Nay,Cf,YZae),(Nay,Cf,YZaf),(N ay,Cf,YZag),(Nay,Cg,YZa),(Nay,Cg,YZb),(Nay,Cg,YZc),(Nay,Cg,YZd),(Nay,Cg,YZe),(Nay ,Cg,YZf),(Nay,Cg,YZg),(Nay,Cg,YZh),(Nay,Cg,YZi),(Nay,Cg,YZj),(Nay,Cg,YZk),(Nay,Cg, YZl),(Nay,Cg,YZm),(Nay,Cg,YZn),(Nay,Cg,YZo),(Nay,Cg,YZp),(Nay,Cg,YZq),(Nay,Cg,Y Zr),(Nay,Cg,YZs),(Nay,Cg,YZt),(Nay,Cg,YZu),(Nay,Cg,YZv),(Nay,Cg,YZw),(Nay,Cg,YZx) ,(Nay,Cg,YZy),(Nay,Cg,YZz),(Nay,Cg,YZaa),(Nay,Cg,YZab),(Nay,Cg,YZac),(Nay,Cg,YZa d),(Nay,Cg,YZae),(Nay,Cg,YZaf),(Nay,Cg,YZag),(Nay,Ch,YZa),(Nay,Ch,YZb),(Nay,Ch,YZ c),(Nay,Ch,YZd),(Nay,Ch,YZe),(Nay,Ch,YZf),(Nay,Ch,YZg),(Nay,Ch,YZh),(Nay,Ch,YZi),( Nay,Ch,YZj),(Nay,Ch,YZk),(Nay,Ch,YZl),(Nay,Ch,YZm),(Nay,Ch,YZn),(Nay,Ch,YZo),(Na y,Ch,YZp),(Nay,Ch,YZq),(Nay,Ch,YZr),(Nay,Ch,YZs),(Nay,Ch,YZt),(Nay,Ch,YZu),(Nay,C h,YZv),(Nay,Ch,YZw),(Nay,Ch,YZx),(Nay,Ch,YZy),(Nay,Ch,YZz),(Nay,Ch,YZaa),(Nay,Ch YZab),(Nay,Ch,YZac),(Nay,Ch,YZad),(Nay,Ch,YZae),(Nay,Ch,YZaf),(Nay,Ch,YZag),(Nay ,Ci,YZa),(Nay,Ci,YZb),(Nay,Ci,YZc),(Nay,Ci,YZd),(Nay,Ci,YZe),(Nay,Ci,YZf),(Nay,Ci,YZ g),(Nay,Ci,YZh),(Nay,Ci,YZi),(Nay,Ci,YZj),(Nay,Ci,YZk),(Nay,Ci,YZl),(Nay,Ci,YZm),(Na y,Ci,YZn),(Nay,Ci,YZo),(Nay,Ci,YZp),(Nay,Ci,YZq),(Nay,Ci,YZr),(Nay,Ci,YZs),(Nay,Ci,Y Zt),(Nay,Ci,YZu),(Nay,Ci,YZv),(Nay,Ci,YZw),(Nay,Ci,YZx),(Nay,Ci,YZy),(Nay,Ci,YZz),( Nay,Ci,YZaa),(Nay,Ci,YZab),(Nay,Ci,YZac),(Nay,Ci,YZad),(Nay,Ci,YZae),(Nay,Ci,YZaf),( Nay,Ci,YZag),(Nay,Cj,YZa),(Nay,Cj,YZb),(Nay,Cj,YZc),(Nay,Cj,YZd),(Nay,Cj,YZe),(Nay, Cj,YZf),(Nay,Cj,YZg),(Nay,Cj,YZh),(Nay,Cj,YZi),(Nay,Cj,YZj),(Nay,Cj,YZk),(Nay,Cj,YZl) ,(Nay,Cj,YZm),(Nay,Cj,YZn),(Nay,Cj,YZo),(Nay,Cj,YZp),(Nay,Cj,YZq),(Nay,Cj,YZr),(Na y,Cj,YZs),(Nay,Cj,YZt),(Nay,Cj,YZu),(Nay,Cj,YZv),(Nay,Cj,YZw),(Nay,Cj,YZx),(Nay,Cj, YZy),(Nay,Cj,YZz),(Nay,Cj,YZaa),(Nay,Cj,YZab),(Nay,Cj,YZac),(Nay,Cj,YZad),(Nay,Cj,Y Zae),(Nay,Cj,YZaf),(Nay,Cj,YZag),(Naz,Ca,YZa),(Naz,Ca,YZb),(Naz,Ca,YZc),(Naz,Ca,YZ d),(Naz,Ca,YZe),(Naz,Ca,YZf),(Naz,Ca,YZg),(Naz,Ca,YZh),(Naz,Ca,YZi),(Naz,Ca,YZj),(N az,Ca,YZk),(Naz,Ca,YZl),(Naz,Ca,YZm),(Naz,Ca,YZn),(Naz,Ca,YZo),(Naz,Ca,YZp),(Naz, Ca,YZq),(Naz,Ca,YZr),(Naz,Ca,YZs),(Naz,Ca,YZt),(Naz,Ca,YZu),(Naz,Ca,YZv),(Naz,Ca,Y Zw),(Naz,Ca,YZx),(Naz,Ca,YZy),(Naz,Ca,YZz),(Naz,Ca,YZaa),(Naz,Ca,YZab),(Naz,Ca,YZ ac),(Naz,Ca,YZad),(Naz,Ca,YZae),(Naz,Ca,YZaf),(Naz,Ca,YZag),(Naz,Cb,YZa),(Naz,Cb,Y Zb),(Naz,Cb,YZc),(Naz,Cb,YZd),(Naz,Cb,YZe),(Naz,Cb,YZf),(Naz,Cb,YZg),(Naz,Cb,YZh), (Naz,Cb,YZi),(Naz,Cb,YZj),(Naz,Cb,YZk),(Naz,Cb,YZl),(Naz,Cb,YZm),(Naz,Cb,YZn),(Na z,Cb,YZo),(Naz,Cb,YZp),(Naz,Cb,YZq),(Naz,Cb,YZr),(Naz,Cb,YZs),(Naz,Cb,YZt),(Naz,Cb ,YZu),(Naz,Cb,YZv),(Naz,Cb,YZw),(Naz,Cb,YZx),(Naz,Cb,YZy),(Naz,Cb,YZz),(Naz,Cb,Y Zaa),(Naz,Cb,YZab),(Naz,Cb,YZac),(Naz,Cb,YZad),(Naz,Cb,YZae),(Naz,Cb,YZaf),(Naz,Cb ,YZag),(Naz,Cc,YZa),(Naz,Cc,YZb),(Naz,Cc,YZc),(Naz,Cc,YZd),(Naz,Cc,YZe),(Naz,Cc,Y Zf),(Naz,Cc,YZg),(Naz,Cc,YZh),(Naz,Cc,YZi),(Naz,Cc,YZj),(Naz,Cc,YZk),(Naz,Cc,YZl),( Naz,Cc,YZm),(Naz,Cc,YZn),(Naz,Cc,YZo),(Naz,Cc,YZp),(Naz,Cc,YZq),(Naz,Cc,YZr),(Naz ,Cc,YZs),(Naz,Cc,YZt),(Naz,Cc,YZu),(Naz,Cc,YZv),(Naz,Cc,YZw),(Naz,Cc,YZx),(Naz,Cc, YZy),(Naz,Cc,YZz),(Naz,Cc,YZaa),(Naz,Cc,YZab),(Naz,Cc,YZac),(Naz,Cc,YZad),(Naz,Cc, YZae),(Naz,Cc,YZaf),(Naz,Cc,YZag),(Naz,Cd,YZa),(Naz,Cd,YZb),(Naz,Cd,YZc),(Naz,Cd, YZd),(Naz,Cd,YZe),(Naz,Cd,YZf),(Naz,Cd,YZg),(Naz,Cd,YZh),(Naz,Cd,YZi),(Naz,Cd,YZj) ,(Naz,Cd,YZk),(Naz,Cd,YZl),(Naz,Cd,YZm),(Naz,Cd,YZn),(Naz,Cd,YZo),(Naz,Cd,YZp),( Naz,Cd,YZq),(Naz,Cd,YZr),(Naz,Cd,YZs),(Naz,Cd,YZt),(Naz,Cd,YZu),(Naz,Cd,YZv),(Naz, Cd,YZw),(Naz,Cd,YZx),(Naz,Cd,YZy),(Naz,Cd,YZz),(Naz,Cd,YZaa),(Naz,Cd,YZab),(Naz, Cd,YZac),(Naz,Cd,YZad),(Naz,Cd,YZae),(Naz,Cd,YZaf),(Naz,Cd,YZag),(Naz,Ce,YZa),(Na z,Ce,YZb),(Naz,Ce,YZc),(Naz,Ce,YZd),(Naz,Ce,YZe),(Naz,Ce,YZf),(Naz,Ce,YZg),(Naz,Ce, Ya),(Naz,Ce,YZi),(Naz,Ce,YZj),(Naz,Ce,YZk),(Naz,Ce,YZl),(Naz,Ce,YZm),(Naz,Ce,YZn) ,(Naz,Ce,YZo),(Naz,Ce,YZp),(Naz,Ce,YZq),(Naz,Ce,YZr),(Naz,Ce,YZs),(Naz,Ce,YZt),(Naz ,Ce,YZu),(Naz,Ce,YZv),(Naz,Ce,YZw),(Naz,Ce,YZx),(Naz,Ce,YZy),(Naz,Ce,YZz),(Naz,Ce, YZaa),(Naz,Ce,YZab),(Naz,Ce,YZac),(Naz,Ce,YZad),(Naz,Ce,YZae),(Naz,Ce,YZaf),(Naz,C e,YZag),(Naz,Cf,YZa),(Naz,Cf,YZb),(Naz,Cf,YZc),(Naz,Cf,YZd),(Naz,Cf,YZe),(Naz,Cf,YZ f),(Naz,Cf,YZg),(Naz,Cf,YZh),(Naz,Cf,YZi),(Naz,Cf,YZj),(Naz,Cf,YZk),(Naz,Cf,YZl),(Naz, Cf,YZm),(Naz,Cf,YZn),(Naz,Cf,YZo),(Naz,Cf,YZp),(Naz,Cf,YZq),(Naz,Cf,YZr),(Naz,Cf,Y Zs),(Naz,Cf,YZt),(Naz,Cf,YZu),(Naz,Cf,YZv),(Naz,Cf,YZw),(Naz,Cf,YZx),(Naz,Cf,YZy),( Naz,Cf,YZz),(Naz,Cf,YZaa),(Naz,Cf,YZab),(Naz,Cf,YZac),(Naz,Cf,YZad),(Naz,Cf,YZae),( Naz,Cf,YZaf),(Naz,Cf,YZag),(Naz,Cg,YZa),(Naz,Cg,YZb),(Naz,Cg,YZc),(Naz,Cg,YZd),(Na z,Cg,YZe),(Naz,Cg,YZf),(Naz,Cg,YZg),(Naz,Cg,YZh),(Naz,Cg,YZi),(Naz,Cg,YZj),(Naz,Cg, YZk),(Naz,Cg,YZl),(Naz,Cg,YZm),(Naz,Cg,YZn),(Naz,Cg,YZo),(Naz,Cg,YZp),(Naz,Cg,YZ q),(Naz,Cg,YZr),(Naz,Cg,YZs),(Naz,Cg,YZt),(Naz,Cg,YZu),(Naz,Cg,YZv),(Naz,Cg,YZw),( Naz,Cg,YZx),(Naz,Cg,YZy),(Naz,Cg,YZz),(Naz,Cg,YZaa),(Naz,Cg,YZab),(Naz,Cg,YZac),( Naz,Cg,YZad),(Naz,Cg,YZae),(Naz,Cg,YZaf),(Naz,Cg,YZag),(Naz,Ch,YZa),(Naz,Ch,YZb), (Naz,Ch,YZc),(Naz,Ch,YZd),(Naz,Ch,YZe),(Naz,Ch,YZf),(Naz,Ch,YZg),(Naz,Ch,YZh),(Na z,Ch,YZi),(Naz,Ch,YZj),(Naz,Ch,YZk),(Naz,Ch,YZl),(Naz,Ch,YZm),(Naz,Ch,YZn),(Naz,C h,YZo),(Naz,Ch,YZp),(Naz,Ch,YZq),(Naz,Ch,YZr),(Naz,Ch,YZs),(Naz,Ch,YZt),(Naz,Ch,Y Zu),(Naz,Ch,YZv),(Naz,Ch,YZw),(Naz,Ch,YZx),(Naz,Ch,YZy),(Naz,Ch,YZz),(Naz,Ch,YZa a),(Naz,Ch,YZab),(Naz,Ch,YZac),(Naz,Ch,YZad),(Naz,Ch,YZae),(Naz,Ch,YZaf),(Naz,Ch,Y Zag),(Naz,Ci,YZa),(Naz,Ci,YZb),(Naz,Ci,YZc),(Naz,Ci,YZd),(Naz,Ci,YZe),(Naz,Ci,YZf),( Naz,Ci,YZg),(Naz,Ci,YZh),(Naz,Ci,YZi),(Naz,Ci,YZj),(Naz,Ci,YZk),(Naz,Ci,YZl),(Naz,Ci, YZm),(Naz,Ci,YZn),(Naz,Ci,YZo),(Naz,Ci,YZp),(Naz,Ci,YZq),(Naz,Ci,YZr),(Naz,Ci,YZs),( Naz,Ci,YZt),(Naz,Ci,YZu),(Naz,Ci,YZv),(Naz,Ci,YZw),(Naz,Ci,YZx),(Naz,Ci,YZy),(Naz,C i,YZz),(Naz,Ci,YZaa),(Naz,Ci,YZab),(Naz,Ci,YZac),(Naz,Ci,YZad),(Naz,Ci,YZae),(Naz,Ci, YZaf),(Naz,Ci,YZag),(Naz,Cj,YZa),(Naz,Cj,YZb),(Naz,Cj,YZc),(Naz,Cj,YZd),(Naz,Cj,YZe) ,(Naz,Cj,YZf),(Naz,Cj,YZg),(Naz,Cj,YZh),(Naz,Cj,YZi),(Naz,Cj,YZj),(Naz,Cj,YZk),(Naz,Cj ,YZl),(Naz,Cj,YZm),(Naz,Cj,YZn),(Naz,Cj,YZo),(Naz,Cj,YZp),(Naz,Cj,YZq),(Naz,Cj,YZr), (Naz,Cj,YZs),(Naz,Cj,YZt),(Naz,Cj,YZu),(Naz,Cj,YZv),(Naz,Cj,YZw),(Naz,Cj,YZx),(Naz,C j,YZy),(Naz,Cj,YZz),(Naz,Cj,YZaa),(Naz,Cj,YZab),(Naz,Cj,YZac),(Naz,Cj,YZad),(Naz,Cj, YZae),(Naz,Cj,YZaf),(Naz,Cj,YZag),(Nba,Ca,YZa),(Nba,Ca,YZb),(Nba,Ca,YZc),(Nba,Ca,Y Zd),(Nba,Ca,YZe),(Nba,Ca,YZf),(Nba,Ca,YZg),(Nba,Ca,YZh),(Nba,Ca,YZi),(Nba,Ca,YZj),( Nba,Ca,YZk),(Nba,Ca,YZl),(Nba,Ca,YZm),(Nba,Ca,YZn),(Nba,Ca,YZo),(Nba,Ca,YZp),(Nb a,Ca,YZq),(Nba,Ca,YZr),(Nba,Ca,YZs),(Nba,Ca,YZt),(Nba,Ca,YZu),(Nba,Ca,YZv),(Nba,Ca ,YZw),(Nba,Ca,YZx),(Nba,Ca,YZy),(Nba,Ca,YZz),(Nba,Ca,YZaa),(Nba,Ca,YZab),(Nba,Ca, YZac),(Nba,Ca,YZad),(Nba,Ca,YZae),(Nba,Ca,YZaf),(Nba,Ca,YZag),(Nba,Cb,YZa),(Nba,C b,YZb),(Nba,Cb,YZc),(Nba,Cb,YZd),(Nba,Cb,YZe),(Nba,Cb,YZf),(Nba,Cb,YZg),(Nba,Cb, YZh),(Nba,Cb,YZi),(Nba,Cb,YZj),(Nba,Cb,YZk),(Nba,Cb,YZl),(Nba,Cb,YZm),(Nba,Cb,YZ n),(Nba,Cb,YZo),(Nba,Cb,YZp),(Nba,Cb,YZq),(Nba,Cb,YZr),(Nba,Cb,YZs),(Nba,Cb,YZt),( Nba,Cb,YZu),(Nba,Cb,YZv),(Nba,Cb,YZw),(Nba,Cb,YZx),(Nba,Cb,YZy),(Nba,Cb,YZz),(N ba,Cb,YZaa),(Nba,Cb,YZab),(Nba,Cb,YZac),(Nba,Cb,YZad),(Nba,Cb,YZae),(Nba,Cb,YZaf) ,(Nba,Cb,YZag),(Nba,Cc,YZa),(Nba,Cc,YZb),(Nba,Cc,YZc),(Nba,Cc,YZd),(Nba,Cc,YZe),( Nba,Cc,YZf),(Nba,Cc,YZg),(Nba,Cc,YZh),(Nba,Cc,YZi),(Nba,Cc,YZj),(Nba,Cc,YZk),(Nba, Cc,YZl),(Nba,Cc,YZm),(Nba,Cc,YZn),(Nba,Cc,YZo),(Nba,Cc,YZp),(Nba,Cc,YZq),(Nba,Cc, YZr),(Nba,Cc,YZs),(Nba,Cc,YZt),(Nba,Cc,YZu),(Nba,Cc,YZv),(Nba,Cc,YZw),(Nba,Cc,YZ x),(Nba,Cc,YZy),(Nba,Cc,YZz),(Nba,Cc,YZaa),(Nba,Cc,YZab),(Nba,Cc,YZac),(Nba,Cc,YZ ad),(Nba,Cc,YZae),(Nba,Cc,YZaf),(Nba,Cc,YZag),(Nba,Cd,YZa),(Nba,Cd,YZb),(Nba,Cd,Y Zc),(Nba,Cd,YZd),(Nba,Cd,YZe),(Nba,Cd,YZf),(Nba,Cd,YZg),(Nba,Cd,YZh),(Nba,Cd,YZi) ,(Nba,Cd,YZj),(Nba,Cd,YZk),(Nba,Cd,YZl),(Nba,Cd,YZm),(Nba,Cd,YZn),(Nba,Cd,YZo),( Nba,Cd,YZp),(Nba,Cd,YZq),(Nba,Cd,YZr),(Nba,Cd,YZs),(Nba,Cd,YZt),(Nba,Cd,YZu),(Nb a,Cd,YZv),(Nba,Cd,YZw),(Nba,Cd,YZx),(Nba,Cd,YZy),(Nba,Cd,YZz),(Nba,Cd,YZaa),(Nba ,Cd,YZab),(Nba,Cd,YZac),(Nba,Cd,YZad),(Nba,Cd,YZae),(Nba,Cd,YZaf),(Nba,Cd,YZag),( Nba,Ce,YZa),(Nba,Ce,YZb),(Nba,Ce,YZc),(Nba,Ce,YZd),(Nba,Ce,YZe),(Nba,Ce,YZf),(Nba ,Ce,YZg),(Nba,Ce,YZh),(Nba,Ce,YZi),(Nba,Ce,YZj),(Nba,Ce,YZk),(Nba,Ce,YZl),(Nba,Ce, YZm),(Nba,Ce,YZn),(Nba,Ce,YZo),(Nba,Ce,YZp),(Nba,Ce,YZq),(Nba,Ce,YZr),(Nba,Ce,YZ s),(Nba,Ce,YZt),(Nba,Ce,YZu),(Nba,Ce,YZv),(Nba,Ce,YZw),(Nba,Ce,YZx),(Nba,Ce,YZy),( Nba,Ce,YZz),(Nba,Ce,YZaa),(Nba,Ce,YZab),(Nba,Ce,YZac),(Nba,Ce,YZad),(Nba,Ce,YZae) ,(Nba,Ce,YZaf),(Nba,Ce,YZag),(Nba,Cf,YZa),(Nba,Cf,YZb),(Nba,Cf,YZc),(Nba,Cf,YZd),( Nba,Cf,YZe),(Nba,Cf,YZf),(Nba,Cf,YZg),(Nba,Cf,YZh),(Nba,Cf,YZi),(Nba,Cf,YZj),(Nba,C f,YZk),(Nba,Cf,YZl),(Nba,Cf,YZm),(Nba,Cf,YZn),(Nba,Cf,YZo),(Nba,Cf,YZp),(Nba,Cf,YZ q),(Nba,Cf,YZr),(Nba,Cf,YZs),(Nba,Cf,YZt),(Nba,Cf,YZu),(Nba,Cf,YZv),(Nba,Cf,YZw),(N ba,Cf,YZx),(Nba,Cf,YZy),(Nba,Cf,YZz),(Nba,Cf,YZaa),(Nba,Cf,YZab),(Nba,Cf,YZac),(Nba ,Cf,YZad),(Nba,Cf,YZae),(Nba,Cf,YZaf),(Nba,Cf,YZag),(Nba,Cg,YZa),(Nba,Cg,YZb),(Nba, Cg,YZc),(Nba,Cg,YZd),(Nba,Cg,YZe),(Nba,Cg,YZf),(Nba,Cg,YZg),(Nba,Cg,YZh),(Nba,Cg ,YZi),(Nba,Cg,YZj),(Nba,Cg,YZk),(Nba,Cg,YZl),(Nba,Cg,YZm),(Nba,Cg,YZn),(Nba,Cg,Y Zo),(Nba,Cg,YZp),(Nba,Cg,YZq),(Nba,Cg,YZr),(Nba,Cg,YZs),(Nba,Cg,YZt),(Nba,Cg,YZu) ,(Nba,Cg,YZv),(Nba,Cg,YZw),(Nba,Cg,YZx),(Nba,Cg,YZy),(Nba,Cg,YZz),(Nba,Cg,YZaa), (Nba,Cg,YZab),(Nba,Cg,YZac),(Nba,Cg,YZad),(Nba,Cg,YZae),(Nba,Cg,YZaf),(Nba,Cg,YZ ag),(Nba,Ch,YZa),(Nba,Ch,YZb),(Nba,Ch,YZc),(Nba,Ch,YZd),(Nba,Ch,YZe),(Nba,Ch,YZf) ,(Nba,Ch,YZg),(Nba,Ch,YZh),(Nba,Ch,YZi),(Nba,Ch,YZj),(Nba,Ch,YZk),(Nba,Ch,YZl),(N ba,Ch,YZm),(Nba,Ch,YZn),(Nba,Ch,YZo),(Nba,Ch,YZp),(Nba,Ch,YZq),(Nba,Ch,YZr),(Nba ,Ch,YZs),(Nba,Ch,YZt),(Nba,Ch,YZu),(Nba,Ch,YZv),(Nba,Ch,YZw),(Nba,Ch,YZx),(Nba,C h,YZy),(Nba,Ch,YZz),(Nba,Ch,YZaa),(Nba,Ch,YZab),(Nba,Ch,YZac),(Nba,Ch,YZad),(Nba, Ch,YZae),(Nba,Ch,YZaf),(Nba,Ch,YZag),(Nba,Ci,YZa),(Nba,Ci,YZb),(Nba,Ci,YZc),(Nba,C i,YZd),(Nba,Ci,YZe),(Nba,Ci,YZf),(Nba,Ci,YZg),(Nba,Ci,YZh),(Nba,Ci,YZi),(Nba,Ci,YZj), (Nba,Ci,YZk),(Nba,Ci,YZl),(Nba,Ci,YZm),(Nba,Ci,YZn),(Nba,Ci,YZo),(Nba,Ci,YZp),(Nba, Ci,YZq),(Nba,Ci,YZr),(Nba,Ci,YZs),(Nba,Ci,YZt),(Nba,Ci,YZu),(Nba,Ci,YZv),(Nba,Ci,YZ w),(Nba,Ci,YZx),(Nba,Ci,YZy),(Nba,Ci,YZz),(Nba,Ci,YZaa),(Nba,Ci,YZab),(Nba,Ci,YZac) ,(Nba,Ci,YZad),(Nba,Ci,YZae),(Nba,Ci,YZaf),(Nba,Ci,YZag),(Nba,Cj,YZa),(Nba,Cj,YZb),( Nba,Cj,YZc),(Nba,Cj,YZd),(Nba,Cj,YZe),(Nba,Cj,YZf),(Nba,Cj,YZg),(Nba,Cj,YZh),(Nba,C j,YZi),(Nba,Cj,YZj),(Nba,Cj,YZk),(Nba,Cj,YZl),(Nba,Cj,YZm),(Nba,Cj,YZn),(Nba,Cj,YZo) ,(Nba,Cj,YZp),(Nba,Cj,YZq),(Nba,Cj,YZr),(Nba,Cj,YZs),(Nba,Cj,YZt),(Nba,Cj,YZu),(Nba, Cj,YZv),(Nba,Cj,YZw),(Nba,Cj,YZx),(Nba,Cj,YZy),(Nba,Cj,YZz),(Nba,Cj,YZaa),(Nba,Cj, YZab),(Nba,Cj,YZac),(Nba,Cj,YZad),(Nba,Cj,YZae),(Nba,Cj,YZaf),(Nba,Cj,YZag),(Nbb,Ca ,YZa),(Nbb,Ca,YZb),(Nbb,Ca,YZc),(Nbb,Ca,YZd),(Nbb,Ca,YZe),(Nbb,Ca,YZf),(Nbb,Ca,Y Zg),(Nbb,Ca,YZh),(Nbb,Ca,YZi),(Nbb,Ca,YZj),(Nbb,Ca,YZk),(Nbb,Ca,YZl),(Nbb,Ca,YZm) ,(Nbb,Ca,YZn),(Nbb,Ca,YZo),(Nbb,Ca,YZp),(Nbb,Ca,YZq),(Nbb,Ca,YZr),(Nbb,Ca,YZs),(N bb,Ca,YZt),(Nbb,Ca,YZu),(Nbb,Ca,YZv),(Nbb,Ca,YZw),(Nbb,Ca,YZx),(Nbb,Ca,YZy),(Nbb ,Ca,YZz),(Nbb,Ca,YZaa),(Nbb,Ca,YZab),(Nbb,Ca,YZac),(Nbb,Ca,YZad),(Nbb,Ca,YZae),(N bb,Ca,YZaf),(Nbb,Ca,YZag),(Nbb,Cb,YZa),(Nbb,Cb,YZb),(Nbb,Cb,YZc),(Nbb,Cb,YZd),(N bb,Cb,YZe),(Nbb,Cb,YZf),(Nbb,Cb,YZg),(Nbb,Cb,YZh),(Nbb,Cb,YZi),(Nbb,Cb,YZj),(Nbb, Cb,YZk),(Nbb,Cb,YZl),(Nbb,Cb,YZm),(Nbb,Cb,YZn),(Nbb,Cb,YZo),(Nbb,Cb,YZp),(Nbb,C b,YZq),(Nbb,Cb,YZr),(Nbb,Cb,YZs),(Nbb,Cb,YZt),(Nbb,Cb,YZu),(Nbb,Cb,YZv),(Nbb,Cb, YZw),(Nbb,Cb,YZx),(Nbb,Cb,YZy),(Nbb,Cb,YZz),(Nbb,Cb,YZaa),(Nbb,Cb,YZab),(Nbb,Cb ,YZac),(Nbb,Cb,YZad),(Nbb,Cb,YZae),(Nbb,Cb,YZaf),(Nbb,Cb,YZag),(Nbb,Cc,YZa),(Nbb, Cc,YZb),(Nbb,Cc,YZc),(Nbb,Cc,YZd),(Nbb,Cc,YZe),(Nbb,Cc,YZf),(Nbb,Cc,YZg),(Nbb,Cc, YZh),(Nbb,Cc,YZi),(Nbb,Cc,YZj),(Nbb,Cc,YZk),(Nbb,Cc,YZl),(Nbb,Cc,YZm),(Nbb,Cc,YZ n),(Nbb,Cc,YZo),(Nbb,Cc,YZp),(Nbb,Cc,YZq),(Nbb,Cc,YZr),(Nbb,Cc,YZs),(Nbb,Cc,YZt),( Nbb,Cc,YZu),(Nbb,Cc,YZv),(Nbb,Cc,YZw),(Nbb,Cc,YZx),(Nbb,Cc,YZy),(Nbb,Cc,YZz),(N bb,Cc,YZaa),(Nbb,Cc,YZab),(Nbb,Cc,YZac),(Nbb,Cc,YZad),(Nbb,Cc,YZae),(Nbb,Cc,YZaf) ,(Nbb,Cc,YZag),(Nbb,Cd,YZa),(Nbb,Cd,YZb),(Nbb,Cd,YZc),(Nbb,Cd,YZd),(Nbb,Cd,YZe), (Nbb,Cd,YZf),(Nbb,Cd,YZg),(Nbb,Cd,YZh),(Nbb,Cd,YZi),(Nbb,Cd,YZj),(Nbb,Cd,YZk),(N bb,Cd,YZl),(Nbb,Cd,YZm),(Nbb,Cd,YZn),(Nbb,Cd,YZo),(Nbb,Cd,YZp),(Nbb,Cd,YZq),(Nb b,Cd,YZr),(Nbb,Cd,YZs),(Nbb,Cd,YZt),(Nbb,Cd,YZu),(Nbb,Cd,YZv),(Nbb,Cd,YZw),(Nbb, Cd,YZx),(Nbb,Cd,YZy),(Nbb,Cd,YZz),(Nbb,Cd,YZaa),(Nbb,Cd,YZab),(Nbb,Cd,YZac),(Nb b,Cd,YZad),(Nbb,Cd,YZae),(Nbb,Cd,YZaf),(Nbb,Cd,YZag),(Nbb,Ce,YZa),(Nbb,Ce,YZb),( Nbb,Ce,YZc),(Nbb,Ce,YZd),(Nbb,Ce,YZe),(Nbb,Ce,YZf),(Nbb,Ce,YZg),(Nbb,Ce,YZh),(Nb b,Ce,YZi),(Nbb,Ce,YZj),(Nbb,Ce,YZk),(Nbb,Ce,YZl),(Nbb,Ce,YZm),(Nbb,Ce,YZn),(Nbb,C e,YZo),(Nbb,Ce,YZp),(Nbb,Ce,YZq),(Nbb,Ce,YZr),(Nbb,Ce,YZs),(Nbb,Ce,YZt),(Nbb,Ce,Y Zu),(Nbb,Ce,YZv),(Nbb,Ce,YZw),(Nbb,Ce,YZx),(Nbb,Ce,YZy),(Nbb,Ce,YZz),(Nbb,Ce,YZ aa),(Nbb,Ce,YZab),(Nbb,Ce,YZac),(Nbb,Ce,YZad),(Nbb,Ce,YZae),(Nbb,Ce,YZaf),(Nbb,Ce, YZag),(Nbb,Cf,YZa),(Nbb,Cf,YZb),(Nbb,Cf,YZc),(Nbb,Cf,YZd),(Nbb,Cf,YZe),(Nbb,Cf,YZ f),(Nbb,Cf,YZg),(Nbb,Cf,YZh),(Nbb,Cf,YZi),(Nbb,Cf,YZj),(Nbb,Cf,YZk),(Nbb,Cf,YZl),(N bb,Cf,YZm),(Nbb,Cf,YZn),(Nbb,Cf,YZo),(Nbb,Cf,YZp),(Nbb,Cf,YZq),(Nbb,Cf,YZr),(Nbb, Cf,YZs),(Nbb,Cf,YZt),(Nbb,Cf,YZu),(Nbb,Cf,YZv),(Nbb,Cf,YZw),(Nbb,Cf,YZx),(Nbb,Cf, YZy),(Nbb,Cf,YZz),(Nbb,Cf,YZaa),(Nbb,Cf,YZab),(Nbb,Cf,YZac),(Nbb,Cf,YZad),(Nbb,Cf, YZae),(Nbb,Cf,YZaf),(Nbb,Cf,YZag),(Nbb,Cg,YZa),(Nbb,Cg,YZb),(Nbb,Cg,YZc),(Nbb,Cg, YZd),(Nbb,Cg,YZe),(Nbb,Cg,YZf),(Nbb,Cg,YZg),(Nbb,Cg,YZh),(Nbb,Cg,YZi),(Nbb,Cg,Y Zj),(Nbb,Cg,YZk),(Nbb,Cg,YZl),(Nbb,Cg,YZm),(Nbb,Cg,YZn),(Nbb,Cg,YZo),(Nbb,Cg,YZ p),(Nbb,Cg,YZq),(Nbb,Cg,YZr),(Nbb,Cg,YZs),(Nbb,Cg,YZt),(Nbb,Cg,YZu),(Nbb,Cg,YZv), (Nbb,Cg,YZw),(Nbb,Cg,YZx),(Nbb,Cg,YZy),(Nbb,Cg,YZz),(Nbb,Cg,YZaa),(Nbb,Cg,YZab) ,(Nbb,Cg,YZac),(Nbb,Cg,YZad),(Nbb,Cg,YZae),(Nbb,Cg,YZaf),(Nbb,Cg,YZag),(Nbb,Ch,Y Za),(Nbb,Ch,YZb),(Nbb,Ch,YZc),(Nbb,Ch,YZd),(Nbb,Ch,YZe),(Nbb,Ch,YZf),(Nbb,Ch,YZg) ,(Nbb,Ch,YZh),(Nbb,Ch,YZi),(Nbb,Ch,YZj),(Nbb,Ch,YZk),(Nbb,Ch,YZl),(Nbb,Ch,YZm),( Nbb,Ch,YZn),(Nbb,Ch,YZo),(Nbb,Ch,YZp),(Nbb,Ch,YZq),(Nbb,Ch,YZr),(Nbb,Ch,YZs),(N bb,Ch,YZt),(Nbb,Ch,YZu),(Nbb,Ch,YZv),(Nbb,Ch,YZw),(Nbb,Ch,YZx),(Nbb,Ch,YZy),(Nb b,Ch,YZz),(Nbb,Ch,YZaa),(Nbb,Ch,YZab),(Nbb,Ch,YZac),(Nbb,Ch,YZad),(Nbb,Ch,YZae),( Nbb,Ch,YZaf),(Nbb,Ch,YZag),(Nbb,Ci,YZa),(Nbb,Ci,YZb),(Nbb,Ci,YZc),(Nbb,Ci,YZd),(N bb,Ci,YZe),(Nbb,Ci,YZf),(Nbb,Ci,YZg),(Nbb,Ci,YZh),(Nbb,Ci,YZi),(Nbb,Ci,YZj),(Nbb,Ci, YZk),(Nbb,Ci,YZl),(Nbb,Ci,YZm),(Nbb,Ci,YZn),(Nbb,Ci,YZo),(Nbb,Ci,YZp),(Nbb,Ci,YZq) ,(Nbb,Ci,YZr),(Nbb,Ci,YZs),(Nbb,Ci,YZt),(Nbb,Ci,YZu),(Nbb,Ci,YZv),(Nbb,Ci,YZw),(Nb b,Ci,YZx),(Nbb,Ci,YZy),(Nbb,Ci,YZz),(Nbb,Ci,YZaa),(Nbb,Ci,YZab),(Nbb,Ci,YZac),(Nbb, Ci,YZad),(Nbb,Ci,YZae),(Nbb,Ci,YZaf),(Nbb,Ci,YZag),(Nbb,Cj,YZa),(Nbb,Cj,YZb),(Nbb, Cj,YZc),(Nbb,Cj,YZd),(Nbb,Cj,YZe),(Nbb,Cj,YZf),(Nbb,Cj,YZg),(Nbb,Cj,YZh),(Nbb,Cj,Y Zi),(Nbb,Cj,YZj),(Nbb,Cj,YZk),(Nbb,Cj,YZl),(Nbb,Cj,YZm),(Nbb,Cj,YZn),(Nbb,Cj,YZo),( Nbb,Cj,YZp),(Nbb,Cj,YZq),(Nbb,Cj,YZr),(Nbb,Cj,YZs),(Nbb,Cj,YZt),(Nbb,Cj,YZu),(Nbb, Cj,YZv),(Nbb,Cj,YZw),(Nbb,Cj,YZx),(Nbb,Cj,YZy),(Nbb,Cj,YZz),(Nbb,Cj,YZaa),(Nbb,Cj, YZab),(Nbb,Cj,YZac),(Nbb,Cj,YZad),(Nbb,Cj,YZae),(Nbb,Cj,YZaf),(Nbb,Cj,YZag),(Nbc,C a,YZa),(Nbc,Ca,YZb),(Nbc,Ca,YZc),(Nbc,Ca,YZd),(Nbc,Ca,YZe),(Nbc,Ca,YZf),(Nbc,Ca,Y Zg),(Nbc,Ca,YZh),(Nbc,Ca,YZi),(Nbc,Ca,YZj),(Nbc,Ca,YZk),(Nbc,Ca,YZl),(Nbc,Ca,YZm), (Nbc,Ca,YZn),(Nbc,Ca,YZo),(Nbc,Ca,YZp),(Nbc,Ca,YZq),(Nbc,Ca,YZr),(Nbc,Ca,YZs),(Nb c,Ca,YZt),(Nbc,Ca,YZu),(Nbc,Ca,YZv),(Nbc,Ca,YZw),(Nbc,Ca,YZx),(Nbc,Ca,YZy),(Nbc,C a,YZz),(Nbc,Ca,YZaa),(Nbc,Ca,YZab),(Nbc,Ca,YZac),(Nbc,Ca,YZad),(Nbc,Ca,YZae),(Nbc, Ca,YZaf),(Nbc,Ca,YZag),(Nbc,Cb,YZa),(Nbc,Cb,YZb),(Nbc,Cb,YZc),(Nbc,Cb,YZd),(Nbc, Cb,YZe),(Nbc,Cb,YZf),(Nbc,Cb,YZg),(Nbc,Cb,YZh),(Nbc,Cb,YZi),(Nbc,Cb,YZj),(Nbc,Cb, YZk),(Nbc,Cb,YZl),(Nbc,Cb,YZm),(Nbc,Cb,YZn),(Nbc,Cb,YZo),(Nbc,Cb,YZp),(Nbc,Cb,Y Zq),(Nbc,Cb,YZr),(Nbc,Cb,YZs),(Nbc,Cb,YZt),(Nbc,Cb,YZu),(Nbc,Cb,YZv),(Nbc,Cb,YZw) ,(Nbc,Cb,YZx),(Nbc,Cb,YZy),(Nbc,Cb,YZz),(Nbc,Cb,YZaa),(Nbc,Cb,YZab),(Nbc,Cb,YZa c),(Nbc,Cb,YZad),(Nbc,Cb,YZae),(Nbc,Cb,YZaf),(Nbc,Cb,YZag),(Nbc,Cc,YZa),(Nbc,Cc,Y Zb),(Nbc,Cc,YZc),(Nbc,Cc,YZd),(Nbc,Cc,YZe),(Nbc,Cc,YZf),(Nbc,Cc,YZg),(Nbc,Cc,YZh), (Nbc,Cc,YZi),(Nbc,Cc,YZj),(Nbc,Cc,YZk),(Nbc,Cc,YZl),(Nbc,Cc,YZm),(Nbc,Cc,YZn),(Nb c,Cc,YZo),(Nbc,Cc,YZp),(Nbc,Cc,YZq),(Nbc,Cc,YZr),(Nbc,Cc,YZs),(Nbc,Cc,YZt),(Nbc,Cc ,YZu),(Nbc,Cc,YZv),(Nbc,Cc,YZw),(Nbc,Cc,YZx),(Nbc,Cc,YZy),(Nbc,Cc,YZz),(Nbc,Cc,Y Zaa),(Nbc,Cc,YZab),(Nbc,Cc,YZac),(Nbc,Cc,YZad),(Nbc,Cc,YZae),(Nbc,Cc,YZaf),(Nbc,Cc ,YZag),(Nbc,Cd,YZa),(Nbc,Cd,YZb),(Nbc,Cd,YZc),(Nbc,Cd,YZd),(Nbc,Cd,YZe),(Nbc,Cd, YZl),(Nbc,Cd,YZg),(Nbc,Cd,YZh),(Nbc,Cd,YZi),(Nbc,Cd,YZj),(Nbc,Cd,YZk),(Nbc,Cd,YZl) ,(Nbc,Cd,YZm),(Nbc,Cd,YZn),(Nbc,Cd,YZo),(Nbc,Cd,YZp),(Nbc,Cd,YZq),(Nbc,Cd,YZr),( Nbc,Cd,YZs),(Nbc,Cd,YZt),(Nbc,Cd,YZu),(Nbc,Cd,YZv),(Nbc,Cd,YZw),(Nbc,Cd,YZx),(Nb c,Cd,YZy),(Nbc,Cd,YZz),(Nbc,Cd,YZaa),(Nbc,Cd,YZab),(Nbc,Cd,YZac),(Nbc,Cd,YZad),(N bc,Cd,YZae),(Nbc,Cd,YZaf),(Nbc,Cd,YZag),(Nbc,Ce,YZa),(Nbc,Ce,YZb),(Nbc,Ce,YZc),(N bc,Ce,YZd),(Nbc,Ce,YZe),(Nbc,Ce,YZf),(Nbc,Ce,YZg),(Nbc,Ce,YZh),(Nbc,Ce,YZi),(Nbc,C e,YZj),(Nbc,Ce,YZk),(Nbc,Ce,YZl),(Nbc,Ce,YZm),(Nbc,Ce,YZn),(Nbc,Ce,YZo),(Nbc,Ce,Y Zp),(Nbc,Ce,YZq),(Nbc,Ce,YZr),(Nbc,Ce,YZs),(Nbc,Ce,YZt),(Nbc,Ce,YZu),(Nbc,Ce,YZv), (Nbc,Ce,YZw),(Nbc,Ce,YZx),(Nbc,Ce,YZy),(Nbc,Ce,YZz),(Nbc,Ce,YZaa),(Nbc,Ce,YZab),( Nbc,Ce,YZac),(Nbc,Ce,YZad),(Nbc,Ce,YZae),(Nbc,Ce,YZaf),(Nbc,Ce,YZag),(Nbc,Cf,YZa), (Nbc,Cf,YZb),(Nbc,Cf,YZc),(Nbc,Cf,YZd),(Nbc,Cf,YZe),(Nbc,Cf,YZf),(Nbc,Cf,YZg),(Nbc, Cf,YZh),(Nbc,Cf,YZi),(Nbc,Cf,YZj),(Nbc,Cf,YZk),(Nbc,Cf,YZl),(Nbc,Cf,YZm),(Nbc,Cf,Y Zn),(Nbc,Cf,YZo),(Nbc,Cf,YZp),(Nbc,Cf,YZq),(Nbc,Cf,YZr),(Nbc,Cf,YZs),(Nbc,Cf,YZt),( Nbc,Cf,YZu),(Nbc,Cf,YZv),(Nbc,Cf,YZw),(Nbc,Cf,YZx),(Nbc,Cf,YZy),(Nbc,Cf,YZz),(Nbc, Cf,YZaa),(Nbc,Cf,YZab),(Nbc,Cf,YZac),(Nbc,Cf,YZad),(Nbc,Cf,YZae),(Nbc,Cf,YZaf),(Nbc ,Cf,YZag),(Nbc,Cg,YZa),(Nbc,Cg,YZb),(Nbc,Cg,YZc),(Nbc,Cg,YZd),(Nbc,Cg,YZe),(Nbc,C g,YZf),(Nbc,Cg,YZg),(Nbc,Cg,YZh),(Nbc,Cg,YZi),(Nbc,Cg,YZj),(Nbc,Cg,YZk),(Nbc,Cg,Y Zl),(Nbc,Cg,YZm),(Nbc,Cg,YZn),(Nbc,Cg,YZo),(Nbc,Cg,YZp),(Nbc,Cg,YZq),(Nbc,Cg,YZr) ,(Nbc,Cg,YZs),(Nbc,Cg,YZt),(Nbc,Cg,YZu),(Nbc,Cg,YZv),(Nbc,Cg,YZw),(Nbc,Cg,YZx),( Nbc,Cg,YZy),(Nbc,Cg,YZz),(Nbc,Cg,YZaa),(Nbc,Cg,YZab),(Nbc,Cg,YZac),(Nbc,Cg,YZad) ,(Nbc,Cg,YZae),(Nbc,Cg,YZaf),(Nbc,Cg,YZag),(Nbc,Ch,YZa),(Nbc,Ch,YZb),(Nbc,Ch,YZc) ,(Nbc,Ch,YZd),(Nbc,Ch,YZe),(Nbc,Ch,YZf),(Nbc,Ch,YZg),(Nbc,Ch,YZh),(Nbc,Ch,YZi),(N bc,Ch,YZj),(Nbc,Ch,YZk),(Nbc,Ch,YZl),(Nbc,Ch,YZm),(Nbc,Ch,YZn),(Nbc,Ch,YZo),(Nbc, Ch,YZp),(Nbc,Ch,YZq),(Nbc,Ch,YZr),(Nbc,Ch,YZs),(Nbc,Ch,YZt),(Nbc,Ch,YZu),(Nbc,Ch, YZv),(Nbc,Ch,YZw),(Nbc,Ch,YZx),(Nbc,Ch,YZy),(Nbc,Ch,YZz),(Nbc,Ch,YZaa),(Nbc,Ch, YZab),(Nbc,Ch,YZac),(Nbc,Ch,YZad),(Nbc,Ch,YZae),(Nbc,Ch,YZaf),(Nbc,Ch,YZag),(Nbc, Ci,YZa),(Nbc,Ci,YZb),(Nbc,Ci,YZc),(Nbc,Ci,YZd),(Nbc,Ci,YZe),(Nbc,Ci,YZf),(Nbc,Ci,YZ g),(Nbc,Ci,YZh),(Nbc,Ci,YZi),(Nbc,Ci,YZj),(Nbc,Ci,YZk),(Nbc,Ci,YZl),(Nbc,Ci,YZm),(Nb c,Ci,YZn),(Nbc,Ci,YZo),(Nbc,Ci,YZp),(Nbc,Ci,YZq),(Nbc,Ci,YZr),(Nbc,Ci,YZs),(Nbc,Ci,Y Zt),(Nbc,Ci,YZu),(Nbc,Ci,YZv),(Nbc,Ci,YZw),(Nbc,Ci,YZx),(Nbc,Ci,YZy),(Nbc,Ci,YZz),( Nbc,Ci,YZaa),(Nbc,Ci,YZab),(Nbc,Ci,YZac),(Nbc,Ci,YZad),(Nbc,Ci,YZae),(Nbc,Ci,YZaf),( Nbc,Ci,YZag),(Nbc,Cj,YZa),(Nbc,Cj,YZb),(Nbc,Cj,YZc),(Nbc,Cj,YZd),(Nbc,Cj,YZe),(Nbc, Cj,YZf),(Nbc,Cj,YZg),(Nbc,Cj,YZh),(Nbc,Cj,YZi),(Nbc,Cj,YZj),(Nbc,Cj,Ya),(Nbc,Cj,YZl) ,(Nbc,Cj,YZm),(Nbc,Cj,YZn),(Nbc,Cj,YZo),(Nbc,Cj,YZp),(Nbc,Cj,YZq),(Nbc,Cj,YZr),(Nb c,Cj,YZs),(Nbc,Cj,YZt),(Nbc,Cj,YZu),(Nbc,Cj,YZv),(Nbc,Cj,YZw),(Nbc,Cj,YZx),(Nbc,Cj, YZy),(Nbc,Cj,YZz),(Nbc,Cj,YZaa),(Nbc,Cj,YZab),(Nbc,Cj,YZac),(Nbc,Cj,YZad),(Nbc,Cj,Y Zae),(Nbc,Cj,YZaf),(Nbc,Cj,YZag),(Nbd,Ca,YZa),(Nbd,Ca,YZb),(Nbd,Ca,YZc),(Nbd,Ca,Y Zd),(Nbd,Ca,YZe),(Nbd,Ca,YZf),(Nbd,Ca,YZg),(Nbd,Ca,YZh),(Nbd,Ca,YZi),(Nbd,Ca,YZj), (Nbd,Ca,YZk),(Nbd,Ca,YZl),(Nbd,Ca,YZm),(Nbd,Ca,YZn),(Nbd,Ca,YZo),(Nbd,Ca,YZp),(N bd,Ca,YZq),(Nbd,Ca,YZr),(Nbd,Ca,YZs),(Nbd,Ca,YZt),(Nbd,Ca,YZu),(Nbd,Ca,YZv),(Nbd, Ca,YZw),(Nbd,Ca,YZx),(Nbd,Ca,YZy),(Nbd,Ca,YZz),(Nbd,Ca,YZaa),(Nbd,Ca,YZab),(Nbd, Ca,YZac),(Nbd,Ca,YZad),(Nbd,Ca,YZae),(Nbd,Ca,YZaf),(Nbd,Ca,YZag),(Nbd,Cb,YZa),(N bd,Cb,YZb),(Nbd,Cb,YZc),(Nbd,Cb,YZd),(Nbd,Cb,YZe),(Nbd,Cb,YZf),(Nbd,Cb,YZg),(Nbd ,Cb,YZh),(Nbd,Cb,YZi),(Nbd,Cb,YZj),(Nbd,Cb,YZk),(Nbd,Cb,YZl),(Nbd,Cb,YZm),(Nbd,C b,YZn),(Nbd,Cb,YZo),(Nbd,Cb,YZp),(Nbd,Cb,YZq),(Nbd,Cb,YZr),(Nbd,Cb,YZs),(Nbd,Cb, YZt),(Nbd,Cb,YZu),(Nbd,Cb,YZv),(Nbd,Cb,YZw),(Nbd,Cb,YZx),(Nbd,Cb,YZy),(Nbd,Cb,Y Zz),(Nbd,Cb,YZaa),(Nbd,Cb,YZab),(Nbd,Cb,YZac),(Nbd,Cb,YZad),(Nbd,Cb,YZae),(Nbd,C b,YZaf),(Nbd,Cb,YZag),(Nbd,Cc,YZa),(Nbd,Cc,YZb),(Nbd,Cc,YZc),(Nbd,Cc,YZd),(Nbd,C c,YZe),(Nbd,Cc,YZf),(Nbd,Cc,YZg),(Nbd,Cc,YZh),(Nbd,Cc,YZi),(Nbd,Cc,YZj),(Nbd,Cc,Y Zk),(Nbd,Cc,YZl),(Nbd,Cc,YZm),(Nbd,Cc,YZn),(Nbd,Cc,YZo),(Nbd,Cc,YZp),(Nbd,Cc,YZ q),(Nbd,Cc,YZr),(Nbd,Cc,YZs),(Nbd,Cc,YZt),(Nbd,Cc,YZu),(Nbd,Cc,YZv),(Nbd,Cc,YZw), (Nbd,Cc,YZx),(Nbd,Cc,YZy),(Nbd,Cc,YZz),(Nbd,Cc,YZaa),(Nbd,Cc,YZab),(Nbd,Cc,YZac) ,(Nbd,Cc,YZad),(Nbd,Cc,YZae),(Nbd,Cc,YZaf),(Nbd,Cc,YZag),(Nbd,Cd,YZa),(Nbd,Cd,YZ b),(Nbd,Cd,YZc),(Nbd,Cd,YZd),(Nbd,Cd,YZe),(Nbd,Cd,YZf),(Nbd,Cd,YZg),(Nbd,Cd,YZh) ,(Nbd,Cd,YZi),(Nbd,Cd,YZj),(Nbd,Cd,YZk),(Nbd,Cd,YZl),(Nbd,Cd,YZm),(Nbd,Cd,YZn),( Nbd,Cd,YZo),(Nbd,Cd,YZp),(Nbd,Cd,YZq),(Nbd,Cd,YZr),(Nbd,Cd,YZs),(Nbd,Cd,YZt),(Nb d,Cd,YZu),(Nbd,Cd,YZv),(Nbd,Cd,YZw),(Nbd,Cd,YZx),(Nbd,Cd,YZy),(Nbd,Cd,YZz),(Nbd ,Cd,YZaa),(Nbd,Cd,YZab),(Nbd,Cd,YZac),(Nbd,Cd,YZad),(Nbd,Cd,YZae),(Nbd,Cd,YZaf),( Nbd,Cd,YZag),(Nbd,Ce,YZa),(Nbd,Ce,YZb),(Nbd,Ce,YZc),(Nbd,Ce,YZd),(Nbd,Ce,YZe),(N bd,Ce,YZf),(Nbd,Ce,YZg),(Nbd,Ce,YZh),(Nbd,Ce,YZi),(Nbd,Ce,YZj),(Nbd,Ce,YZk),(Nbd, Ce,YZl),(Nbd,Ce,YZm),(Nbd,Ce,YZn),(Nbd,Ce,YZo),(Nbd,Ce,YZp),(Nbd,Ce,YZq),(Nbd,C e,YZr),(Nbd,Ce,YZs),(Nbd,Ce,YZt),(Nbd,Ce,YZu),(Nbd,Ce,YZv),(Nbd,Ce,YZw),(Nbd,Ce, YZx),(Nbd,Ce,YZy),(Nbd,Ce,YZz),(Nbd,Ce,YZaa),(Nbd,Ce,YZab),(Nbd,Ce,YZac),(Nbd,Ce ,YZad),(Nbd,Ce,YZae),(Nbd,Ce,YZaf),(Nbd,Ce,YZag),(Nbd,Cf,YZa),(Nbd,Cf,YZb),(Nbd,C f,YZc),(Nbd,Cf,YZd),(Nbd,Cf,YZe),(Nbd,Cf,YZf),(Nbd,Cf,YZg),(Nbd,Cf,YZh),(Nbd,Cf,YZ i),(Nbd,Cf,YZj),(Nbd,Cf,YZk),(Nbd,Cf,YZl),(Nbd,Cf,YZm),(Nbd,Cf,YZn),(Nbd,Cf,YZo),(N bd,Cf,YZp),(Nbd,Cf,YZq),(Nbd,Cf,YZr),(Nbd,Cf,YZs),(Nbd,Cf,YZt),(Nbd,Cf,YZu),(Nbd,Cf ,YZv),(Nbd,Cf,YZw),(Nbd,Cf,YZx),(Nbd,Cf,YZy),(Nbd,Cf,YZz),(Nbd,Cf,YZaa),(Nbd,Cf,Y Zab),(Nbd,Cf,YZac),(Nbd,Cf,YZad),(Nbd,Cf,YZae),(Nbd,Cf,YZaf),(Nbd,Cf,YZag),(Nbd,Cg ,YZa),(Nbd,Cg,YZb),(Nbd,Cg,YZc),(Nbd,Cg,YZd),(Nbd,Cg,YZe),(Nbd,Cg,YZf),(Nbd,Cg,Y Zg),(Nbd,Cg,YZh),(Nbd,Cg,YZi),(Nbd,Cg,YZj),(Nbd,Cg,YZk),(Nbd,Cg,YZl),(Nbd,Cg,YZm) ,(Nbd,Cg,YZn),(Nbd,Cg,YZo),(Nbd,Cg,YZp),(Nbd,Cg,YZq),(Nbd,Cg,YZr),(Nbd,Cg,YZs),( Nbd,Cg,YZt),(Nbd,Cg,YZu),(Nbd,Cg,YZv),(Nbd,Cg,YZw),(Nbd,Cg,YZx),(Nbd,Cg,YZy),(N bd,Cg,YZz),(Nbd,Cg,YZaa),(Nbd,Cg,YZab),(Nbd,Cg,YZac),(Nbd,Cg,YZad),(Nbd,Cg,YZae) ,(Nbd,Cg,YZaf),(Nbd,Cg,YZag),(Nbd,Ch,YZa),(Nbd,Ch,YZb),(Nbd,Ch,YZc),(Nbd,Ch,YZd) ,(Nbd,Ch,YZe),(Nbd,Ch,YZf),(Nbd,Ch,YZg),(Nbd,Ch,YZh),(Nbd,Ch,YZi),(Nbd,Ch,YZj),(N bd,Ch,YZk),(Nbd,Ch,YZl),(Nbd,Ch,YZm),(Nbd,Ch,YZn),(Nbd,Ch,YZo),(Nbd,Ch,YZp),(Nb d,Ch,YZq),(Nbd,Ch,YZr),(Nbd,Ch,YZs),(Nbd,Ch,YZt),(Nbd,Ch,YZu),(Nbd,Ch,YZv),(Nbd, Ch,YZw),(Nbd,Ch,YZx),(Nbd,Ch,YZy),(Nbd,Ch,YZz),(Nbd,Ch,YZaa),(Nbd,Ch,YZab),(Nbd ,Ch,YZac),(Nbd,Ch,YZad),(Nbd,Ch,YZae),(Nbd,Ch,YZaf),(Nbd,Ch,YZag),(Nbd,Ci,YZa),(N bd,Ci,YZb),(Nbd,Ci,YZc),(Nbd,Ci,YZd),(Nbd,Ci,YZe),(Nbd,Ci,YZf),(Nbd,Ci,YZg),(Nbd,Ci ,YZh),(Nbd,Ci,YZi),(Nbd,Ci,YZj),(Nbd,Ci,YZk),(Nbd,Ci,YZl),(Nbd,Ci,YZm),(Nbd,Ci,YZn) ,(Nbd,Ci,YZo),(Nbd,Ci,YZp),(Nbd,Ci,YZq),(Nbd,Ci,YZr),(Nbd,Ci,YZs),(Nbd,Ci,YZt),(Nbd, Ci,YZu),(Nbd,Ci,YZv),(Nbd,Ci,YZw),(Nbd,Ci,YZx),(Nbd,Ci,YZy),(Nbd,Ci,YZz),(Nbd,Ci,Y Zaa),(Nbd,Ci,YZab),(Nbd,Ci,YZac),(Nbd,Ci,YZad),(Nbd,Ci,YZae),(Nbd,Ci,YZaf),(Nbd,Ci, YZag),(Nbd,Cj,YZa),(Nbd,Cj,YZb),(Nbd,Cj,YZc),(Nbd,Cj,YZd),(Nbd,Cj,YZe),(Nbd,Cj,YZf) ,(Nbd,Cj,YZg),(Nbd,Cj,YZh),(Nbd,Cj,YZi),(Nbd,Cj,YZj),(Nbd,Cj,YZk),(Nbd,Cj,YZl),(Nbd ,Cj,YZm),(Nbd,Cj,YZn),(Nbd,Cj,YZo),(Nbd,Cj,YZp),(Nbd,Cj,YZq),(Nbd,Cj,YZr),(Nbd,Cj, YZs),(Nbd,Cj,YZt),(Nbd,Cj,YZu),(Nbd,Cj,YZv),(Nbd,Cj,YZw),(Nbd,Cj,YZx),(Nbd,Cj,YZy) ,(Nbd,Cj,YZz),(Nbd,Cj,YZaa),(Nbd,Cj,YZab),(Nbd,Cj,YZac),(Nbd,Cj,YZad),(Nbd,Cj,YZae) ,(Nbd,Cj,YZaf),(Nbd,Cj,YZag),(Nbe,Ca,YZa),(Nbe,Ca,YZb),(Nbe,Ca,YZc),(Nbe,Ca,YZd),( Nbe,Ca,YZe),(Nbe,Ca,YZf),(Nbe,Ca,YZg),(Nbe,Ca,YZh),(Nbe,Ca,YZi),(Nbe,Ca,YZj),(Nbe, Ca,YZk),(Nbe,Ca,YZl),(Nbe,Ca,YZm),(Nbe,Ca,YZn),(Nbe,Ca,YZo),(Nbe,Ca,YZp),(Nbe,Ca, YZq),(Nbe,Ca,YZr),(Nbe,Ca,YZs),(Nbe,Ca,YZt),(Nbe,Ca,YZu),(Nbe,Ca,YZv),(Nbe,Ca,YZ w),(Nbe,Ca,YZx),(Nbe,Ca,YZy),(Nbe,Ca,YZz),(Nbe,Ca,YZaa),(Nbe,Ca,YZab),(Nbe,Ca,YZa c),(Nbe,Ca,YZad),(Nbe,Ca,YZae),(Nbe,Ca,YZaf),(Nbe,Ca,YZag),(Nbe,Cb,YZa),(Nbe,Cb,Y Zb),(Nbe,Cb,YZc),(Nbe,Cb,YZd),(Nbe,Cb,YZe),(Nbe,Cb,YZf),(Nbe,Cb,YZg),(Nbe,Cb,YZh) ,(Nbe,Cb,YZi),(Nbe,Cb,YZj),(Nbe,Cb,Ya),(Nbe,Cb,YZl),(Nbe,Cb,YZm),(Nbe,Cb,YZn),( Nbe,Cb,YZo),(Nbe,Cb,YZp),(Nbe,Cb,YZq),(Nbe,Cb,YZr),(Nbe,Cb,YZs),(Nbe,Cb,YZt),(Nb e,Cb,YZu),(Nbe,Cb,YZv),(Nbe,Cb,YZw),(Nbe,Cb,YZx),(Nbe,Cb,YZy),(Nbe,Cb,YZz),(Nbe, Cb,YZaa),(Nbe,Cb,YZab),(Nbe,Cb,YZac),(Nbe,Cb,YZad),(Nbe,Cb,YZae),(Nbe,Cb,YZaf),( Nbe,Cb,YZag),(Nbe,Cc,YZa),(Nbe,Cc,YZb),(Nbe,Cc,YZc),(Nbe,Cc,YZd),(Nbe,Cc,YZe),(N be,Cc,YZf),(Nbe,Cc,YZg),(Nbe,Cc,YZh),(Nbe,Cc,YZi),(Nbe,Cc,YZj),(Nbe,Cc,YZk),(Nbe,C c,YZl),(Nbe,Cc,YZm),(Nbe,Cc,YZn),(Nbe,Cc,YZo),(Nbe,Cc,YZp),(Nbe,Cc,YZq),(Nbe,Cc, YZr),(Nbe,Cc,YZs),(Nbe,Cc,YZt),(Nbe,Cc,YZu),(Nbe,Cc,YZv),(Nbe,Cc,YZw),(Nbe,Cc,YZ x),(Nbe,Cc,YZy),(Nbe,Cc,YZz),(Nbe,Cc,YZaa),(Nbe,Cc,YZab),(Nbe,Cc,YZac),(Nbe,Cc,YZ ad),(Nbe,Cc,YZae),(Nbe,Cc,YZaf),(Nbe,Cc,YZag),(Nbe,Cd,YZa),(Nbe,Cd,YZb),(Nbe,Cd,Y Zc),(Nbe,Cd,YZd),(Nbe,Cd,YZe),(Nbe,Cd,YZf),(Nbe,Cd,YZg),(Nbe,Cd,YZh),(Nbe,Cd,YZi) ,(Nbe,Cd,YZj),(Nbe,Cd,YZk),(Nbe,Cd,YZl),(Nbe,Cd,YZm),(Nbe,Cd,YZn),(Nbe,Cd,YZo),( Nbe,Cd,YZp),(Nbe,Cd,YZq),(Nbe,Cd,YZr),(Nbe,Cd,YZs),(Nbe,Cd,YZt),(Nbe,Cd,YZu),(Nb e,Cd,YZv),(Nbe,Cd,YZw),(Nbe,Cd,YZx),(Nbe,Cd,YZy),(Nbe,Cd,YZz),(Nbe,Cd,YZaa),(Nbe ,Cd,YZab),(Nbe,Cd,YZac),(Nbe,Cd,YZad),(Nbe,Cd,YZae),(Nbe,Cd,YZaf),(Nbe,Cd,YZag),( Nbe,Ce,YZa),(Nbe,Ce,YZb),(Nbe,Ce,YZc),(Nbe,Ce,YZd),(Nbe,Ce,YZe),(Nbe,Ce,YZf),(Nbe ,Ce,YZg),(Nbe,Ce,YZh),(Nbe,Ce,YZi),(Nbe,Ce,YZj),(Nbe,Ce,YZk),(Nbe,Ce,YZl),(Nbe,Ce, YZm),(Nbe,Ce,YZn),(Nbe,Ce,YZo),(Nbe,Ce,YZp),(Nbe,Ce,YZq),(Nbe,Ce,YZr),(Nbe,Ce,YZ s),(Nbe,Ce,YZt),(Nbe,Ce,YZu),(Nbe,Ce,YZv),(Nbe,Ce,YZw),(Nbe,Ce,YZx),(Nbe,Ce,YZy),( Nbe,Ce,YZz),(Nbe,Ce,YZaa),(Nbe,Ce,YZab),(Nbe,Ce,YZac),(Nbe,Ce,YZad),(Nbe,Ce,YZae) ,(Nbe,Ce,YZaf),(Nbe,Ce,YZag),(Nbe,Cf,YZa),(Nbe,Cf,YZb),(Nbe,Cf,YZc),(Nbe,Cf,YZd),( Nbe,Cf,YZe),(Nbe,Cf,YZf),(Nbe,Cf,YZg),(Nbe,Cf,YZh),(Nbe,Cf,YZi),(Nbe,Cf,YZj),(Nbe,C f,YZk),(Nbe,Cf,YZl),(Nbe,Cf,YZm),(Nbe,Cf,YZn),(Nbe,Cf,YZo),(Nbe,Cf,YZp),(Nbe,Cf,YZ q),(Nbe,Cf,YZr),(Nbe,Cf,YZs),(Nbe,Cf,YZt),(Nbe,Cf,YZu),(Nbe,Cf,YZv),(Nbe,Cf,YZw),(N be,Cf,YZx),(Nbe,Cf,YZy),(Nbe,Cf,YZz),(Nbe,Cf,YZaa),(Nbe,Cf,YZab),(Nbe,Cf,YZac),(Nbe ,Cf,YZad),(Nbe,Cf,YZae),(Nbe,Cf,YZaf),(Nbe,Cf,YZag),(Nbe,Cg,YZa),(Nbe,Cg,YZb),(Nbe, Cg,YZc),(Nbe,Cg,YZd),(Nbe,Cg,YZe),(Nbe,Cg,YZf),(Nbe,Cg,YZg),(Nbe,Cg,YZh),(Nbe,Cg ,YZi),(Nbe,Cg,YZj),(Nbe,Cg,YZk),(Nbe,Cg,YZl),(Nbe,Cg,YZm),(Nbe,Cg,YZn),(Nbe,Cg,Y Zo),(Nbe,Cg,YZp),(Nbe,Cg,YZq),(Nbe,Cg,YZr),(Nbe,Cg,YZs),(Nbe,Cg,YZt),(Nbe,Cg,YZu) ,(Nbe,Cg,YZv),(Nbe,Cg,YZw),(Nbe,Cg,YZx),(Nbe,Cg,YZy),(Nbe,Cg,YZz),(Nbe,Cg,YZaa), (Nbe,Cg,YZab),(Nbe,Cg,YZac),(Nbe,Cg,YZad),(Nbe,Cg,YZae),(Nbe,Cg,YZaf),(Nbe,Cg,YZ ag),(Nbe,Ch,YZa),(Nbe,Ch,YZb),(Nbe,Ch,YZc),(Nbe,Ch,YZd),(Nbe,Ch,YZe),(Nbe,Ch,YZf) ,(Nbe,Ch,YZg),(Nbe,Ch,YZh),(Nbe,Ch,YZi),(Nbe,Ch,YZj),(Nbe,Ch,YZk),(Nbe,Ch,YZl),(N be,Ch,YZm),(Nbe,Ch,YZn),(Nbe,Ch,YZo),(Nbe,Ch,YZp),(Nbe,Ch,YZq),(Nbe,Ch,YZr),(Nbe ,Ch,YZs),(Nbe,Ch,YZt),(Nbe,Ch,YZu),(Nbe,Ch,YZv),(Nbe,Ch,YZw),(Nbe,Ch,YZx),(Nbe,C h,YZy),(Nbe,Ch,YZz),(Nbe,Ch,YZaa),(Nbe,Ch,YZab),(Nbe,Ch,YZac),(Nbe,Ch,YZad),(Nbe, Ch,YZae),(Nbe,Ch,YZaf),(Nbe,Ch,YZag),(Nbe,Ci,YZa),(Nbe,Ci,YZb),(Nbe,Ci,YZc),(Nbe,C i,YZd),(Nbe,Ci,YZe),(Nbe,Ci,YZf),(Nbe,Ci,YZg),(Nbe,Ci,YZh),(Nbe,Ci,YZi),(Nbe,Ci,YZj), (Nbe,Ci,YZk),(Nbe,Ci,YZl),(Nbe,Ci,YZm),(Nbe,Ci,YZn),(Nbe,Ci,YZo),(Nbe,Ci,YZp),(Nbe, Ci,YZq),(Nbe,Ci,YZr),(Nbe,Ci,YZs),(Nbe,Ci,YZt),(Nbe,Ci,YZu),(Nbe,Ci,YZv),(Nbe,Ci,YZ w),(Nbe,Ci,YZx),(Nbe,Ci,YZy),(Nbe,Ci,YZz),(Nbe,Ci,YZaa),(Nbe,Ci,YZab),(Nbe,Ci,YZac) ,(Nbe,Ci,YZad),(Nbe,Ci,YZae),(Nbe,Ci,YZaf),(Nbe,Ci,YZag),(Nbe,Cj,YZa),(Nbe,Cj,YZb),( Nbe,Cj,YZc),(Nbe,Cj,YZd),(Nbe,Cj,YZe),(Nbe,Cj,YZf),(Nbe,Cj,YZg),(Nbe,Cj,YZh),(Nbe,C j,YZi),(Nbe,Cj,YZj),(Nbe,Cj,YZk),(Nbe,Cj,YZl),(Nbe,Cj,YZm),(Nbe,Cj,YZn),(Nbe,Cj,YZo) ,(Nbe,Cj,YZp),(Nbe,Cj,YZq),(Nbe,Cj,YZr),(Nbe,Cj,YZs),(Nbe,Cj,YZt),(Nbe,Cj,YZu),(Nbe, Cj,YZv),(Nbe,Cj,YZw),(Nbe,Cj,YZx),(Nbe,Cj,YZy),(Nbe,Cj,YZz),(Nbe,Cj,YZaa),(Nbe,Cj, YZab),(Nbe,Cj,YZac),(Nbe,Cj,YZad),(Nbe,Cj,YZae),(Nbe,Cj,YZaf),(Nbe,Cj,YZag),(Nbf,Ca, YZa),(Nbf,Ca,YZb),(Nbf,Ca,YZc),(Nbf,Ca,YZd),(Nbf,Ca,YZe),(Nbf,Ca,YZf),(Nbf,Ca,YZg), (Nbf,Ca,YZh),(Nbf,Ca,YZi),(Nbf,Ca,YZj),(Nbf,Ca,YZk),(Nbf,Ca,YZl),(Nbf,Ca,YZm),(Nbf, Ca,YZn),(Nbf,Ca,YZo),(Nbf,Ca,YZp),(Nbf,Ca,YZq),(Nbf,Ca,YZr),(Nbf,Ca,YZs),(Nbf,Ca,Y Zt),(Nbf,Ca,YZu),(Nbf,Ca,YZv),(Nbf,Ca,YZw),(Nbf,Ca,YZx),(Nbf,Ca,YZy),(Nbf,Ca,YZz),( Nbf,Ca,YZaa),(Nbf,Ca,YZab),(Nbf,Ca,YZac),(Nbf,Ca,YZad),(Nbf,Ca,YZae),(Nbf,Ca,YZaf), (Nbf,Ca,YZag),(Nbf,Cb,YZa),(Nbf,Cb,YZb),(Nbf,Cb,YZc),(Nbf,Cb,YZd),(Nbf,Cb,YZe),(Nb f,Cb,YZf),(Nbf,Cb,YZg),(Nbf,Cb,YZh),(Nbf,Cb,YZi),(Nbf,Cb,YZj),(Nbf,Cb,YZk),(Nbf,Cb, YZl),(Nbf,Cb,YZm),(Nbf,Cb,YZn),(Nbf,Cb,YZo),(Nbf,Cb,YZp),(Nbf,Cb,YZq),(Nbf,Cb,YZr) ,(Nbf,Cb,YZs),(Nbf,Cb,YZt),(Nbf,Cb,YZu),(Nbf,Cb,YZv),(Nbf,Cb,YZw),(Nbf,Cb,YZx),(N bf,Cb,YZy),(Nbf,Cb,YZz),(Nbf,Cb,YZaa),(Nbf,Cb,YZab),(Nbf,Cb,YZac),(Nbf,Cb,YZad),(N bf,Cb,YZae),(Nbf,Cb,YZaf),(Nbf,Cb,YZag),(Nbf,Cc,YZa),(Nbf,Cc,YZb),(Nbf,Cc,YZc),(Nbf ,Cc,YZd),(Nbf,Cc,YZe),(Nbf,Cc,YZf),(Nbf,Cc,YZg),(Nbf,Cc,YZh),(Nbf,Cc,YZi),(Nbf,Cc,Y Zj),(Nbf,Cc,YZk),(Nbf,Cc,YZl),(Nbf,Cc,YZm),(Nbf,Cc,YZn),(Nbf,Cc,YZo),(Nbf,Cc,YZp),( Nbf,Cc,YZq),(Nbf,Cc,YZr),(Nbf,Cc,YZs),(Nbf,Cc,YZt),(Nbf,Cc,YZu),(Nbf,Cc,YZv),(Nbf,C c,YZw),(Nbf,Cc,YZx),(Nbf,Cc,YZy),(Nbf,Cc,YZz),(Nbf,Cc,YZaa),(Nbf,Cc,YZab),(Nbf,Cc, YZac),(Nbf,Cc,YZad),(Nbf,Cc,YZae),(Nbf,Cc,YZaf),(Nbf,Cc,YZag),(Nbf,Cd,YZa),(Nbf,Cd, YZb),(Nbf,Cd,YZc),(Nbf,Cd,YZd),(Nbf,Cd,YZe),(Nbf,Cd,YZf),(Nbf,Cd,YZg),(Nbf,Cd,YZh) ,(Nbf,Cd,YZi),(Nbf,Cd,YZj),(Nbf,Cd,YZk),(Nbf,Cd,YZl),(Nbf,Cd,YZm),(Nbf,Cd,YZn),(Nb f,Cd,YZo),(Nbf,Cd,YZp),(Nbf,Cd,YZq),(Nbf,Cd,YZr),(Nbf,Cd,YZs),(Nbf,Cd,YZt),(Nbf,Cd, YZu),(Nbf,Cd,YZv),(Nbf,Cd,YZw),(Nbf,Cd,YZx),(Nbf,Cd,YZy),(Nbf,Cd,YZz),(Nbf,Cd,YZ aa),(Nbf,Cd,YZab),(Nbf,Cd,YZac),(Nbf,Cd,YZad),(Nbf,Cd,YZae),(Nbf,Cd,YZaf),(Nbf,Cd,Y Zag),(Nbf,Ce,YZa),(Nbf,Ce,YZb),(Nbf,Ce,YZc),(Nbf,Ce,YZd),(Nbf,Ce,YZe),(Nbf,Ce,YZf), (Nbf,Ce,YZg),(Nbf,Ce,YZh),(Nbf,Ce,YZi),(Nbf,Ce,YZj),(Nbf,Ce,YZk),(Nbf,Ce,YZl),(Nbf, Ce,YZm),(Nbf,Ce,YZn),(Nbf,Ce,YZo),(Nbf,Ce,YZp),(Nbf,Ce,YZq),(Nbf,Ce,YZr),(Nbf,Ce, YZs),(Nbf,Ce,YZt),(Nbf,Ce,YZu),(Nbf,Ce,YZv),(Nbf,Ce,YZw),(Nbf,Ce,YZx),(Nbf,Ce,YZy) ,(Nbf,Ce,YZz),(Nbf,Ce,YZaa),(Nbf,Ce,YZab),(Nbf,Ce,YZac),(Nbf,Ce,YZad),(Nbf,Ce,YZae) ,(Nbf,Ce,YZaf),(Nbf,Ce,YZag),(Nbf,Cf,YZa),(Nbf,Cf,YZb),(Nbf,Cf,YZc),(Nbf,Cf,YZd),(Nb f,Cf,YZe),(Nbf,Cf,YZf),(Nbf,Cf,YZg),(Nbf,Cf,YZh),(Nbf,Cf,YZi),(Nbf,Cf,YZj),(Nbf,Cf,YZ k),(Nbf,Cf,YZl),(Nbf,Cf,YZm),(Nbf,Cf,YZn),(Nbf,Cf,YZo),(Nbf,Cf,YZp),(Nbf,Cf,YZq),(Nb f,Cf,YZr),(Nbf,Cf,YZs),(Nbf,Cf,Ya),(Nbf,Cf,YZu),(Nbf,Cf,YZv),(Nbf,Cf,YZw),(Nbf,Cf,Y Zx),(Nbf,Cf,YZy),(Nbf,Cf,YZz),(Nbf,Cf,YZaa),(Nbf,Cf,YZab),(Nbf,Cf,YZac),(Nbf,Cf,YZad) ,(Nbf,Cf,YZae),(Nbf,Cf,YZaf),(Nbf,Cf,YZag),(Nbf,Cg,YZa),(Nbf,Cg,YZb),(Nbf,Cg,YZc),( Nbf,Cg,YZd),(Nbf,Cg,YZe),(Nbf,Cg,YZf),(Nbf,Cg,YZg),(Nbf,Cg,YZh),(Nbf,Cg,YZi),(Nbf, Cg,YZj),(Nbf,Cg,YZk),(Nbf,Cg,YZl),(Nbf,Cg,YZm),(Nbf,Cg,YZn),(Nbf,Cg,YZo),(Nbf,Cg, YZp),(Nbf,Cg,YZq),(Nbf,Cg,YZr),(Nbf,Cg,YZs),(Nbf,Cg,YZt),(Nbf,Cg,YZu),(Nbf,Cg,YZv) ,(Nbf,Cg,YZw),(Nbf,Cg,YZx),(Nbf,Cg,YZy),(Nbf,Cg,YZz),(Nbf,Cg,YZaa),(Nbf,Cg,YZab),( Nbf,Cg,YZac),(Nbf,Cg,YZad),(Nbf,Cg,YZae),(Nbf,Cg,YZaf),(Nbf,Cg,YZag),(Nbf,Ch,YZa), (Nbf,Ch,YZb),(Nbf,Ch,YZc),(Nbf,Ch,YZd),(Nbf,Ch,YZe),(Nbf,Ch,YZf),(Nbf,Ch,YZg),(Nbf ,Ch,YZh),(Nbf,Ch,YZi),(Nbf,Ch,YZj),(Nbf,Ch,YZk),(Nbf,Ch,YZl),(Nbf,Ch,YZm),(Nbf,Ch, YZn),(Nbf,Ch,YZo),(Nbf,Ch,YZp),(Nbf,Ch,YZq),(Nbf,Ch,YZr),(Nbf,Ch,YZs),(Nbf,Ch,YZt) ,(Nbf,Ch,YZu),(Nbf,Ch,YZv),(Nbf,Ch,YZw),(Nbf,Ch,YZx),(Nbf,Ch,YZy),(Nbf,Ch,YZz),(N bf,Ch,YZaa),(Nbf,Ch,YZab),(Nbf,Ch,YZac),(Nbf,Ch,YZad),(Nbf,Ch,YZae),(Nbf,Ch,YZaf),( Nbf,Ch,YZag),(Nbf,Ci,YZa),(Nbf,Ci,YZb),(Nbf,Ci,YZc),(Nbf,Ci,YZd),(Nbf,Ci,YZe),(Nbf,C i,YZf),(Nbf,Ci,YZg),(Nbf,Ci,YZh),(Nbf,Ci,YZi),(Nbf,Ci,YZj),(Nbf,Ci,YZk),(Nbf,Ci,YZl),( Nbf,Ci,YZm),(Nbf,Ci,YZn),(Nbf,Ci,YZo),(Nbf,Ci,YZp),(Nbf,Ci,YZq),(Nbf,Ci,YZr),(Nbf,Ci, YZs),(Nbf,Ci,YZt),(Nbf,Ci,YZu),(Nbf,Ci,YZv),(Nbf,Ci,YZw),(Nbf,Ci,YZx),(Nbf,Ci,YZy),( Nbf,Ci,YZz),(Nbf,Ci,YZaa),(Nbf,Ci,YZab),(Nbf,Ci,YZac),(Nbf,Ci,YZad),(Nbf,Ci,YZae),(N bf,Ci,YZaf),(Nbf,Ci,YZag),(Nbf,Cj,YZa),(Nbf,Cj,YZb),(Nbf,Cj,YZc),(Nbf,Cj,YZd),(Nbf,Cj, YZe),(Nbf,Cj,YZf),(Nbf,Cj,YZg),(Nbf,Cj,YZh),(Nbf,Cj,YZi),(Nbf,Cj,YZj),(Nbf,Cj,YZk),(N bf,Cj,YZl),(Nbf,Cj,YZm),(Nbf,Cj,YZn),(Nbf,Cj,YZo),(Nbf,Cj,YZp),(Nbf,Cj,YZq),(Nbf,Cj,Y Zr),(Nbf,Cj,YZs),(Nbf,Cj,YZt),(Nbf,Cj,YZu),(Nbf,Cj,YZv),(Nbf,Cj,YZw),(Nbf,Cj,YZx),(Nb f,Cj,YZy),(Nbf,Cj,YZz),(Nbf,Cj,YZaa),(Nbf,Cj,YZab),(Nbf,Cj,YZac),(Nbf,Cj,YZad),(Nbf,C j,YZae),(Nbf,Cj,YZaf),(Nbf,Cj,YZag),(Nbg,Ca,YZa),(Nbg,Ca,YZb),(Nbg,Ca,YZc),(Nbg,Ca, YZd),(Nbg,Ca,YZe),(Nbg,Ca,YZf),(Nbg,Ca,YZg),(Nbg,Ca,YZh),(Nbg,Ca,YZi),(Nbg,Ca,YZ j),(Nbg,Ca,YZk),(Nbg,Ca,YZl),(Nbg,Ca,YZm),(Nbg,Ca,YZn),(Nbg,Ca,YZo),(Nbg,Ca,YZp), (Nbg,Ca,YZq),(Nbg,Ca,YZr),(Nbg,Ca,YZs),(Nbg,Ca,YZt),(Nbg,Ca,YZu),(Nbg,Ca,YZv),(Nb g,Ca,YZw),(Nbg,Ca,YZx),(Nbg,Ca,YZy),(Nbg,Ca,YZz),(Nbg,Ca,YZaa),(Nbg,Ca,YZab),(Nb g,Ca,YZac),(Nbg,Ca,YZad),(Nbg,Ca,YZae),(Nbg,Ca,YZaf),(Nbg,Ca,YZag),(Nbg,Cb,YZa),( Nbg,Cb,YZb),(Nbg,Cb,YZc),(Nbg,Cb,YZd),(Nbg,Cb,YZe),(Nbg,Cb,YZf),(Nbg,Cb,YZg),(N bg,Cb,YZh),(Nbg,Cb,YZi),(Nbg,Cb,YZj),(Nbg,Cb,Ya),(Nbg,Cb,YZl),(Nbg,Cb,YZm),(Nbg ,Cb,YZn),(Nbg,Cb,YZo),(Nbg,Cb,YZp),(Nbg,Cb,YZq),(Nbg,Cb,YZr),(Nbg,Cb,YZs),(Nbg,C b,YZt),(Nbg,Cb,YZu),(Nbg,Cb,YZv),(Nbg,Cb,YZw),(Nbg,Cb,YZx),(Nbg,Cb,YZy),(Nbg,Cb, YZz),(Nbg,Cb,YZaa),(Nbg,Cb,YZab),(Nbg,Cb,YZac),(Nbg,Cb,YZad),(Nbg,Cb,YZae),(Nbg, Cb,YZaf),(Nbg,Cb,YZag),(Nbg,Cc,YZa),(Nbg,Cc,YZb),(Nbg,Cc,YZc),(Nbg,Cc,YZd),(Nbg, Cc,YZe),(Nbg,Cc,YZf),(Nbg,Cc,YZg),(Nbg,Cc,YZh),(Nbg,Cc,YZi),(Nbg,Cc,YZj),(Nbg,Cc, YZk),(Nbg,Cc,YZl),(Nbg,Cc,YZm),(Nbg,Cc,YZn),(Nbg,Cc,YZo),(Nbg,Cc,YZp),(Nbg,Cc,Y Zq),(Nbg,Cc,YZr),(Nbg,Cc,YZs),(Nbg,Cc,YZt),(Nbg,Cc,YZu),(Nbg,Cc,YZv),(Nbg,Cc,YZw) ,(Nbg,Cc,YZx),(Nbg,Cc,YZy),(Nbg,Cc,YZz),(Nbg,Cc,YZaa),(Nbg,Cc,YZab),(Nbg,Cc,YZa c),(Nbg,Cc,YZad),(Nbg,Cc,YZae),(Nbg,Cc,YZaf),(Nbg,Cc,YZag),(Nbg,Cd,YZa),(Nbg,Cd,Y Zb),(Nbg,Cd,YZc),(Nbg,Cd,YZd),(Nbg,Cd,YZe),(Nbg,Cd,YZf),(Nbg,Cd,YZg),(Nbg,Cd,YZ h),(Nbg,Cd,YZi),(Nbg,Cd,YZj),(Nbg,Cd,YZk),(Nbg,Cd,YZl),(Nbg,Cd,YZm),(Nbg,Cd,YZn), (Nbg,Cd,YZo),(Nbg,Cd,YZp),(Nbg,Cd,YZq),(Nbg,Cd,YZr),(Nbg,Cd,YZs),(Nbg,Cd,YZt),(N bg,Cd,YZu),(Nbg,Cd,YZv),(Nbg,Cd,YZw),(Nbg,Cd,YZx),(Nbg,Cd,YZy),(Nbg,Cd,YZz),(Nb g,Cd,YZaa),(Nbg,Cd,YZab),(Nbg,Cd,YZac),(Nbg,Cd,YZad),(Nbg,Cd,YZae),(Nbg,Cd,YZaf), (Nbg,Cd,YZag),(Nbg,Ce,YZa),(Nbg,Ce,YZb),(Nbg,Ce,YZc),(Nbg,Ce,YZd),(Nbg,Ce,YZe),( Nbg,Ce,YZf),(Nbg,Ce,YZg),(Nbg,Ce,YZh),(Nbg,Ce,YZi),(Nbg,Ce,YZj),(Nbg,Ce,YZk),(Nbg ,Ce,YZl),(Nbg,Ce,YZm),(Nbg,Ce,YZn),(Nbg,Ce,YZo),(Nbg,Ce,YZp),(Nbg,Ce,YZq),(Nbg,C e,YZr),(Nbg,Ce,YZs),(Nbg,Ce,YZt),(Nbg,Ce,YZu),(Nbg,Ce,YZv),(Nbg,Ce,YZw),(Nbg,Ce, YZx),(Nbg,Ce,YZy),(Nbg,Ce,YZz),(Nbg,Ce,YZaa),(Nbg,Ce,YZab),(Nbg,Ce,YZac),(Nbg,Ce ,YZad),(Nbg,Ce,YZae),(Nbg,Ce,YZaf),(Nbg,Ce,YZag),(Nbg,Cf,YZa),(Nbg,Cf,YZb),(Nbg,C f,YZc),(Nbg,Cf,YZd),(Nbg,Cf,YZe),(Nbg,Cf,YZf),(Nbg,Cf,YZg),(Nbg,Cf,YZh),(Nbg,Cf,YZ i),(Nbg,Cf,YZj),(Nbg,Cf,YZk),(Nbg,Cf,YZl),(Nbg,Cf,YZm),(Nbg,Cf,YZn),(Nbg,Cf,YZo),(N bg,Cf,YZp),(Nbg,Cf,YZq),(Nbg,Cf,YZr),(Nbg,Cf,YZs),(Nbg,Cf,YZt),(Nbg,Cf,YZu),(Nbg,Cf ,YZv),(Nbg,Cf,YZw),(Nbg,Cf,YZx),(Nbg,Cf,YZy),(Nbg,Cf,YZz),(Nbg,Cf,YZaa),(Nbg,Cf,Y Zab),(Nbg,Cf,YZac),(Nbg,Cf,YZad),(Nbg,Cf,YZae),(Nbg,Cf,YZaf),(Nbg,Cf,YZag),(Nbg,Cg ,YZa),(Nbg,Cg,YZb),(Nbg,Cg,YZc),(Nbg,Cg,YZd),(Nbg,Cg,YZe),(Nbg,Cg,YZf),(Nbg,Cg,Y Zg),(Nbg,Cg,YZh),(Nbg,Cg,YZi),(Nbg,Cg,YZj),(Nbg,Cg,YZk),(Nbg,Cg,YZl),(Nbg,Cg,YZm) ,(Nbg,Cg,YZn),(Nbg,Cg,YZo),(Nbg,Cg,YZp),(Nbg,Cg,YZq),(Nbg,Cg,YZr),(Nbg,Cg,YZs),( Nbg,Cg,YZt),(Nbg,Cg,YZu),(Nbg,Cg,YZv),(Nbg,Cg,YZw),(Nbg,Cg,YZx),(Nbg,Cg,YZy),(N bg,Cg,YZz),(Nbg,Cg,YZaa),(Nbg,Cg,YZab),(Nbg,Cg,YZac),(Nbg,Cg,YZad),(Nbg,Cg,YZae) ,(Nbg,Cg,YZaf),(Nbg,Cg,YZag),(Nbg,Ch,YZa),(Nbg,Ch,YZb),(Nbg,Ch,YZc),(Nbg,Ch,YZd) ,(Nbg,Ch,YZe),(Nbg,Ch,YZf),(Nbg,Ch,YZg),(Nbg,Ch,YZh),(Nbg,Ch,YZi),(Nbg,Ch,YZj),(N bg,Ch,YZk),(Nbg,Ch,YZl),(Nbg,Ch,YZm),(Nbg,Ch,YZn),(Nbg,Ch,YZo),(Nbg,Ch,YZp),(Nb g,Ch,YZq),(Nbg,Ch,YZr),(Nbg,Ch,YZs),(Nbg,Ch,YZt),(Nbg,Ch,YZu),(Nbg,Ch,YZv),(Nbg, Ch,YZw),(Nbg,Ch,YZx),(Nbg,Ch,YZy),(Nbg,Ch,YZz),(Nbg,Ch,YZaa),(Nbg,Ch,YZab),(Nbg ,Ch,YZac),(Nbg,Ch,YZad),(Nbg,Ch,YZae),(Nbg,Ch,YZaf),(Nbg,Ch,YZag),(Nbg,Ci,YZa),(N bg,Ci,YZb),(Nbg,Ci,YZc),(Nbg,Ci,YZd),(Nbg,Ci,YZe),(Nbg,Ci,YZf),(Nbg,Ci,YZg),(Nbg,Ci ,YZh),(Nbg,Ci,YZi),(Nbg,Ci,YZj),(Nbg,Ci,YZk),(Nbg,Ci,YZl),(Nbg,Ci,YZm),(Nbg,Ci,YZn) ,(Nbg,Ci,YZo),(Nbg,Ci,YZp),(Nbg,Ci,YZq),(Nbg,Ci,YZr),(Nbg,Ci,YZs),(Nbg,Ci,YZt),(Nbg, Ci,YZu),(Nbg,Ci,YZv),(Nbg,Ci,YZw),(Nbg,Ci,YZx),(Nbg,Ci,YZy),(Nbg,Ci,YZz),(Nbg,Ci,Y Zaa),(Nbg,Ci,YZab),(Nbg,Ci,YZac),(Nbg,Ci,YZad),(Nbg,Ci,YZae),(Nbg,Ci,YZaf),(Nbg,Ci, YZag),(Nbg,Cj,YZa),(Nbg,Cj,YZb),(Nbg,Cj,YZc),(Nbg,Cj,YZd),(Nbg,Cj,YZe),(Nbg,Cj,YZf) ,(Nbg,Cj,YZg),(Nbg,Cj,YZh),(Nbg,Cj,YZi),(Nbg,Cj,YZj),(Nbg,Cj,YZk),(Nbg,Cj,YZl),(Nbg ,Cj,YZm),(Nbg,Cj,YZn),(Nbg,Cj,YZo),(Nbg,Cj,YZp),(Nbg,Cj,YZq),(Nbg,Cj,YZr),(Nbg,Cj, YZs),(Nbg,Cj,YZt),(Nbg,Cj,YZu),(Nbg,Cj,YZv),(Nbg,Cj,YZw),(Nbg,Cj,YZx),(Nbg,Cj,YZy) ,(Nbg,Cj,YZz),(Nbg,Cj,YZaa),(Nbg,Cj,YZab),(Nbg,Cj,YZac),(Nbg,Cj,YZad),(Nbg,Cj,YZae) ,(Nbg,Cj,YZaf),(Nbg,Cj,YZag),(Nbh,Ca,YZa),(Nbh,Ca,YZb),(Nbh,Ca,YZc),(Nbh,Ca,YZd),( Nbh,Ca,YZe),(Nbh,Ca,YZf),(Nbh,Ca,YZg),(Nbh,Ca,YZh),(Nbh,Ca,YZi),(Nbh,Ca,YZj),(Nbh ,Ca,YZk),(Nbh,Ca,YZl),(Nbh,Ca,YZm),(Nbh,Ca,YZn),(Nbh,Ca,YZo),(Nbh,Ca,YZp),(Nbh,C a,YZq),(Nbh,Ca,YZr),(Nbh,Ca,YZs),(Nbh,Ca,YZt),(Nbh,Ca,YZu),(Nbh,Ca,YZv),(Nbh,Ca,Y Zw),(Nbh,Ca,YZx),(Nbh,Ca,YZy),(Nbh,Ca,YZz),(Nbh,Ca,YZaa),(Nbh,Ca,YZab),(Nbh,Ca,Y Zac),(Nbh,Ca,YZad),(Nbh,Ca,YZae),(Nbh,Ca,YZaf),(Nbh,Ca,YZag),(Nbh,Cb,YZa),(Nbh,Cb ,YZb),(Nbh,Cb,YZc),(Nbh,Cb,YZd),(Nbh,Cb,YZe),(Nbh,Cb,YZf),(Nbh,Cb,YZg),(Nbh,Cb,Y Zh),(Nbh,Cb,YZi),(Nbh,Cb,YZj),(Nbh,Cb,YZk),(Nbh,Cb,YZl),(Nbh,Cb,YZm),(Nbh,Cb,YZn) ,(Nbh,Cb,YZo),(Nbh,Cb,YZp),(Nbh,Cb,YZq),(Nbh,Cb,YZr),(Nbh,Cb,YZs),(Nbh,Cb,YZt),( Nbh,Cb,YZu),(Nbh,Cb,YZv),(Nbh,Cb,YZw),(Nbh,Cb,YZx),(Nbh,Cb,YZy),(Nbh,Cb,YZz),( Nbh,Cb,YZaa),(Nbh,Cb,YZab),(Nbh,Cb,YZac),(Nbh,Cb,YZad),(Nbh,Cb,YZae),(Nbh,Cb,YZ af),(Nbh,Cb,YZag),(Nbh,Cc,YZa),(Nbh,Cc,YZb),(Nbh,Cc,YZc),(Nbh,Cc,YZd),(Nbh,Cc,YZe) ,(Nbh,Cc,YZf),(Nbh,Cc,YZg),(Nbh,Cc,YZh),(Nbh,Cc,YZi),(Nbh,Cc,YZj),(Nbh,Cc,YZk),(N bh,Cc,YZl),(Nbh,Cc,YZm),(Nbh,Cc,YZn),(Nbh,Cc,YZo),(Nbh,Cc,YZp),(Nbh,Cc,YZq),(Nbh ,Cc,YZr),(Nbh,Cc,YZs),(Nbh,Cc,YZt),(Nbh,Cc,YZu),(Nbh,Cc,YZv),(Nbh,Cc,YZw),(Nbh,Cc ,YZx),(Nbh,Cc,YZy),(Nbh,Cc,YZz),(Nbh,Cc,YZaa),(Nbh,Cc,YZab),(Nbh,Cc,YZac),(Nbh,C c,YZad),(Nbh,Cc,YZae),(Nbh,Cc,YZaf),(Nbh,Cc,YZag),(Nbh,Cd,YZa),(Nbh,Cd,YZb),(Nbh, Cd,YZc),(Nbh,Cd,YZd),(Nbh,Cd,YZe),(Nbh,Cd,YZf),(Nbh,Cd,YZg),(Nbh,Cd,YZh),(Nbh,C d,YZi),(Nbh,Cd,YZj),(Nbh,Cd,YZk),(Nbh,Cd,YZl),(Nbh,Cd,YZm),(Nbh,Cd,YZn),(Nbh,Cd, YZo),(Nbh,Cd,YZp),(Nbh,Cd,YZq),(Nbh,Cd,YZr),(Nbh,Cd,YZs),(Nbh,Cd,YZt),(Nbh,Cd,Y Zu),(Nbh,Cd,YZv),(Nbh,Cd,YZw),(Nbh,Cd,YZx),(Nbh,Cd,YZy),(Nbh,Cd,YZz),(Nbh,Cd,YZ aa),(Nbh,Cd,YZab),(Nbh,Cd,YZac),(Nbh,Cd,YZad),(Nbh,Cd,YZae),(Nbh,Cd,YZaf),(Nbh,Cd ,YZag),(Nbh,Ce,YZa),(Nbh,Ce,YZb),(Nbh,Ce,YZc),(Nbh,Ce,YZd),(Nbh,Ce,YZe),(Nbh,Ce, YZf),(Nbh,Ce,YZg),(Nbh,Ce,YZh),(Nbh,Ce,YZi),(Nbh,Ce,YZj),(Nbh,Ce,YZk),(Nbh,Ce,YZl) ,(Nbh,Ce,YZm),(Nbh,Ce,YZn),(Nbh,Ce,YZo),(Nbh,Ce,YZp),(Nbh,Ce,YZq),(Nbh,Ce,YZr),( Nbh,Ce,YZs),(Nbh,Ce,YZt),(Nbh,Ce,YZu),(Nbh,Ce,YZv),(Nbh,Ce,YZw),(Nbh,Ce,YZx),(Nb h,Ce,YZy),(Nbh,Ce,YZz),(Nbh,Ce,YZaa),(Nbh,Ce,YZab),(Nbh,Ce,YZac),(Nbh,Ce,YZad),(N bh,Ce,YZae),(Nbh,Ce,YZaf),(Nbh,Ce,YZag),(Nbh,Cf,YZa),(Nbh,Cf,YZb),(Nbh,Cf,YZc),(N bh,Cf,YZd),(Nbh,Cf,YZe),(Nbh,Cf,YZf),(Nbh,Cf,YZg),(Nbh,Cf,YZh),(Nbh,Cf,YZi),(Nbh,C f,YZj),(Nbh,Cf,YZk),(Nbh,Cf,YZl),(Nbh,Cf,YZm),(Nbh,Cf,YZn),(Nbh,Cf,YZo),(Nbh,Cf,YZ p),(Nbh,Cf,YZq),(Nbh,Cf,YZr),(Nbh,Cf,YZs),(Nbh,Cf,YZt),(Nbh,Cf,YZu),(Nbh,Cf,YZv),(N bh,Cf,YZw),(Nbh,Cf,YZx),(Nbh,Cf,YZy),(Nbh,Cf,YZz),(Nbh,Cf,YZaa),(Nbh,Cf,YZab),(Nb h,Cf,YZac),(Nbh,Cf,YZad),(Nbh,Cf,YZae),(Nbh,Cf,YZaf),(Nbh,Cf,YZag),(Nbh,Cg,YZa),(N bh,Cg,YZb),(Nbh,Cg,YZc),(Nbh,Cg,YZd),(Nbh,Cg,YZe),(Nbh,Cg,YZf),(Nbh,Cg,YZg),(Nbh ,Cg,YZh),(Nbh,Cg,YZi),(Nbh,Cg,YZj),(Nbh,Cg,YZk),(Nbh,Cg,YZl),(Nbh,Cg,YZm),(Nbh,C g,YZn),(Nbh,Cg,YZo),(Nbh,Cg,YZp),(Nbh,Cg,YZq),(Nbh,Cg,YZr),(Nbh,Cg,YZs),(Nbh,Cg, YZt),(Nbh,Cg,YZu),(Nbh,Cg,YZv),(Nbh,Cg,YZw),(Nbh,Cg,YZx),(Nbh,Cg,YZy),(Nbh,Cg,Y Zz),(Nbh,Cg,YZaa),(Nbh,Cg,YZab),(Nbh,Cg,YZac),(Nbh,Cg,YZad),(Nbh,Cg,YZae),(Nbh,C g,YZaf),(Nbh,Cg,YZag),(Nbh,Ch,YZa),(Nbh,Ch,YZb),(Nbh,Ch,YZc),(Nbh,Ch,YZd),(Nbh,C h,YZe),(Nbh,Ch,YZf),(Nbh,Ch,YZg),(Nbh,Ch,YZh),(Nbh,Ch,YZi),(Nbh,Ch,YZj),(Nbh,Ch, YZk),(Nbh,Ch,YZl),(Nbh,Ch,YZm),(Nbh,Ch,YZn),(Nbh,Ch,YZo),(Nbh,Ch,YZp),(Nbh,Ch,Y Zq),(Nbh,Ch,YZr),(Nbh,Ch,YZs),(Nbh,Ch,YZt),(Nbh,Ch,YZu),(Nbh,Ch,YZv),(Nbh,Ch,YZ w),(Nbh,Ch,YZx),(Nbh,Ch,YZy),(Nbh,Ch,YZz),(Nbh,Ch,YZaa),(Nbh,Ch,YZab),(Nbh,Ch,Y Zac),(Nbh,Ch,YZad),(Nbh,Ch,YZae),(Nbh,Ch,YZaf),(Nbh,Ch,YZag),(Nbh,Ci,YZa),(Nbh,Ci, YZb),(Nbh,Ci,YZc),(Nbh,Ci,YZd),(Nbh,Ci,YZe),(Nbh,Ci,YZf),(Nbh,Ci,YZg),(Nbh,Ci,YZh) ,(Nbh,Ci,YZi),(Nbh,Ci,YZj),(Nbh,Ci,YZk),(Nbh,Ci,YZl),(Nbh,Ci,YZm),(Nbh,Ci,YZn),(Nbh ,Ci,YZo),(Nbh,Ci,YZp),(Nbh,Ci,YZq),(Nbh,Ci,YZr),(Nbh,Ci,YZs),(Nbh,Ci,YZt),(Nbh,Ci,Y Zu),(Nbh,Ci,YZv),(Nbh,Ci,YZw),(Nbh,Ci,YZx),(Nbh,Ci,YZy),(Nbh,Ci,YZz),(Nbh,Ci,YZaa) ,(Nbh,Ci,YZab),(Nbh,Ci,YZac),(Nbh,Ci,YZad),(Nbh,Ci,YZae),(Nbh,Ci,YZaf),(Nbh,Ci,YZag) ,(Nbh,Cj,YZa),(Nbh,Cj,YZb),(Nbh,Cj,YZc),(Nbh,Cj,YZd),(Nbh,Cj,YZe),(Nbh,Cj,YZf),(Nb h,Cj,YZg),(Nbh,Cj,YZh),(Nbh,Cj,YZi),(Nbh,Cj,YZj),(Nbh,Cj,Ya),(Nbh,Cj,YZl),(Nbh,Cj,Y Zm),(Nbh,Cj,YZn),(Nbh,Cj,YZo),(Nbh,Cj,YZp),(Nbh,Cj,YZq),(Nbh,Cj,YZr),(Nbh,Cj,YZs),( Nbh,Cj,YZt),(Nbh,Cj,YZu),(Nbh,Cj,YZv),(Nbh,Cj,YZw),(Nbh,Cj,YZx),(Nbh,Cj,YZy),(Nbh, Cj,YZz),(Nbh,Cj,YZaa),(Nbh,Cj,YZab),(Nbh,Cj,YZac),(Nbh,Cj,YZad),(Nbh,Cj,YZae),(Nbh, Cj,YZaf),(Nbh,Cj,YZag),(Nbi,Ca,YZa),(Nbi,Ca,YZb),(Nbi,Ca,YZc),(Nbi,Ca,YZd),(Nbi,Ca, YZe),(Nbi,Ca,YZf),(Nbi,Ca,YZg),(Nbi,Ca,YZh),(Nbi,Ca,YZi),(Nbi,Ca,YZj),(Nbi,Ca,YZk),( Nbi,Ca,YZl),(Nbi,Ca,YZm),(Nbi,Ca,YZn),(Nbi,Ca,YZo),(Nbi,Ca,YZp),(Nbi,Ca,YZq),(Nbi, Ca,YZr),(Nbi,Ca,YZs),(Nbi,Ca,YZt),(Nbi,Ca,YZu),(Nbi,Ca,YZu),(Nbi,Ca,YZw),(Nbi,Ca,YZ x),(Nbi,Ca,YZy),(Nbi,Ca,YZz),(Nbi,Ca,YZaa),(Nbi,Ca,YZab),(Nbi,Ca,YZac),(Nbi,Ca,YZad) ,(Nbi,Ca,YZae),(Nbi,Ca,YZaf),(Nbi,Ca,YZag),(Nbi,Cb,YZa),(Nbi,Cb,YZb),(Nbi,Cb,YZc),( Nbi,Cb,YZd),(Nbi,Cb,YZe),(Nbi,Cb,YZf),(Nbi,Cb,YZg),(Nbi,Cb,YZh),(Nbi,Cb,YZi),(Nbi,C b,YZj),(Nbi,Cb,YZk),(Nbi,Cb,YZl),(Nbi,Cb,YZm),(Nbi,Cb,YZn),(Nbi,Cb,YZo),(Nbi,Cb,YZ p),(Nbi,Cb,YZq),(Nbi,Cb,YZr),(Nbi,Cb,YZs),(Nbi,Cb,YZt),(Nbi,Cb,YZu),(Nbi,Cb,YZv),(N bi,Cb,YZw),(Nbi,Cb,YZx),(Nbi,Cb,YZy),(Nbi,Cb,YZz),(Nbi,Cb,YZaa),(Nbi,Cb,YZab),(Nbi, Cb,YZac),(Nbi,Cb,YZad),(Nbi,Cb,YZae),(Nbi,Cb,YZaf),(Nbi,Cb,YZag),(Nbi,Cc,YZa),(Nbi, Cc,YZb),(Nbi,Cc,YZc),(Nbi,Cc,YZd),(Nbi,Cc,YZe),(Nbi,Cc,YZf),(Nbi,Cc,YZg),(Nbi,Cc,YZ h),(Nbi,Cc,YZi),(Nbi,Cc,YZj),(Nbi,Cc,YZk),(Nbi,Cc,YZl),(Nbi,Cc,YZm),(Nbi,Cc,YZn),(Nb i,Cc,YZo),(Nbi,Cc,YZp),(Nbi,Cc,YZq),(Nbi,Cc,YZr),(Nbi,Cc,YZs),(Nbi,Cc,YZt),(Nbi,Cc,Y Zu),(Nbi,Cc,YZv),(Nbi,Cc,YZw),(Nbi,Cc,YZx),(Nbi,Cc,YZy),(Nbi,Cc,YZz),(Nbi,Cc,YZaa), (Nbi,Cc,YZab),(Nbi,Cc,YZac),(Nbi,Cc,YZad),(Nbi,Cc,YZae),(Nbi,Cc,YZaf),(Nbi,Cc,YZag), (Nbi,Cd,YZa),(Nbi,Cd,YZb),(Nbi,Cd,YZc),(Nbi,Cd,YZd),(Nbi,Cd,YZe),(Nbi,Cd,YZf),(Nbi, Cd,YZg),(Nbi,Cd,YZh),(Nbi,Cd,YZi),(Nbi,Cd,YZj),(Nbi,Cd,YZk),(Nbi,Cd,YZl),(Nbi,Cd,YZ m),(Nbi,Cd,YZn),(Nbi,Cd,YZo),(Nbi,Cd,YZp),(Nbi,Cd,YZq),(Nbi,Cd,YZr),(Nbi,Cd,YZs),( Nbi,Cd,YZt),(Nbi,Cd,YZu),(Nbi,Cd,YZv),(Nbi,Cd,YZw),(Nbi,Cd,YZx),(Nbi,Cd,YZy),(Nbi, Cd,YZz),(Nbi,Cd,YZaa),(Nbi,Cd,YZab),(Nbi,Cd,YZac),(Nbi,Cd,YZad),(Nbi,Cd,YZae),(Nbi, Cd,YZaf),(Nbi,Cd,YZag),(Nbi,Ce,YZa),(Nbi,Ce,YZb),(Nbi,Ce,YZc),(Nbi,Ce,YZd),(Nbi,Ce, YZe),(Nbi,Ce,YZf),(Nbi,Ce,YZg),(Nbi,Ce,YZh),(Nbi,Ce,YZi),(Nbi,Ce,YZj),(Nbi,Ce,YZk),( Nbi,Ce,YZl),(Nbi,Ce,YZm),(Nbi,Ce,YZn),(Nbi,Ce,YZo),(Nbi,Ce,YZp),(Nbi,Ce,YZq),(Nbi, Ce,YZr),(Nbi,Ce,YZs),(Nbi,Ce,YZt),(Nbi,Ce,YZu),(Nbi,Ce,YZv),(Nbi,Ce,YZw),(Nbi,Ce,YZ x),(Nbi,Ce,YZy),(Nbi,Ce,YZz),(Nbi,Ce,YZaa),(Nbi,Ce,YZab),(Nbi,Ce,YZac),(Nbi,Ce,YZad) ,(Nbi,Ce,YZae),(Nbi,Ce,YZaf),(Nbi,Ce,YZag),(Nbi,Cf,YZa),(Nbi,Cf,YZb),(Nbi,Cf,YZc),(N bi,Cf,YZd),(Nbi,Cf,YZe),(Nbi,Cf,YZf),(Nbi,Cf,YZg),(Nbi,Cf,YZh),(Nbi,Cf,YZi),(Nbi,Cf,Y Zj),(Nbi,Cf,YZk),(Nbi,Cf,YZl),(Nbi,Cf,YZm),(Nbi,Cf,YZn),(Nbi,Cf,YZo),(Nbi,Cf,YZp),(Nb i,Cf,YZq),(Nbi,Cf,YZr),(Nbi,Cf,YZs),(Nbi,Cf,YZt),(Nbi,Cf,YZu),(Nbi,Cf,YZv),(Nbi,Cf,YZ w),(Nbi,Cf,YZx),(Nbi,Cf,YZy),(Nbi,Cf,YZz),(Nbi,Cf,YZaa),(Nbi,Cf,YZab),(Nbi,Cf,YZac),( Nbi,Cf,YZad),(Nbi,Cf,YZae),(Nbi,Cf,YZaf),(Nbi,Cf,YZag),(Nbi,Cg,YZa),(Nbi,Cg,YZb),(Nb i,Cg,YZc),(Nbi,Cg,YZd),(Nbi,Cg,YZe),(Nbi,Cg,YZf),(Nbi,Cg,YZg),(Nbi,Cg,YZh),(Nbi,Cg, YZi),(Nbi,Cg,YZj),(Nbi,Cg,YZk),(Nbi,Cg,YZl),(Nbi,Cg,YZm),(Nbi,Cg,YZn),(Nbi,Cg,YZo), (Nbi,Cg,YZp),(Nbi,Cg,YZq),(Nbi,Cg,YZr),(Nbi,Cg,YZs),(Nbi,Cg,YZt),(Nbi,Cg,YZu),(Nbi, Cg,YZv),(Nbi,Cg,YZw),(Nbi,Cg,YZx),(Nbi,Cg,YZy),(Nbi,Cg,YZz),(Nbi,Cg,YZaa),(Nbi,Cg, YZab),(Nbi,Cg,YZac),(Nbi,Cg,YZad),(Nbi,Cg,YZae),(Nbi,Cg,YZaf),(Nbi,Cg,YZag),(Nbi,C h,YZa),(Nbi,Ch,YZb),(Nbi,Ch,YZc),(Nbi,Ch,YZd),(Nbi,Ch,YZe),(Nbi,Ch,YZf),(Nbi,Ch,YZ g),(Nbi,Ch,YZh),(Nbi,Ch,YZi),(Nbi,Ch,YZj),(Nbi,Ch,YZk),(Nbi,Ch,YZl),(Nbi,Ch,YZm),(N bi,Ch,YZn),(Nbi,Ch,YZo),(Nbi,Ch,YZp),(Nbi,Ch,YZq),(Nbi,Ch,YZr),(Nbi,Ch,YZs),(Nbi,Ch ,YZt),(Nbi,Ch,YZu),(Nbi,Ch,YZv),(Nbi,Ch,YZw),(Nbi,Ch,YZx),(Nbi,Ch,YZy),(Nbi,Ch,YZz) ,(Nbi,Ch,YZaa),(Nbi,Ch,YZab),(Nbi,Ch,YZac),(Nbi,Ch,YZad),(Nbi,Ch,YZae),(Nbi,Ch,YZa f),(Nbi,Ch,YZag),(Nbi,Ci,YZa),(Nbi,Ci,YZb),(Nbi,Ci,YZc),(Nbi,Ci,YZd),(Nbi,Ci,YZe),(Nbi Nbi,Ci,YZm),(Nbi,Ci,YZn),(Nbi,Ci,YZo),(Nbi,Ci,YZp),(Nbi,Ci,YZq),(Nbi,Ci,YZr),(Nbi,Ci, YZs),(Nbi,Ci,YZt),(Nbi,Ci,YZu),(Nbi,Ci,YZv),(Nbi,Ci,YZw),(Nbi,Ci,YZx),(Nbi,Ci,YZy),(N bi,Ci,YZz),(Nbi,Ci,YZaa),(Nbi,Ci,YZab),(Nbi,Ci,YZac),(Nbi,Ci,YZad),(Nbi,Ci,YZae),(Nbi, Ci,YZaf),(Nbi,Ci,YZag),(Nbi,Cj,YZa),(Nbi,Cj,YZb),(Nbi,Cj,YZc),(Nbi,Cj,YZd),(Nbi,Cj,YZ e),(Nbi,Cj,YZf),(Nbi,Cj,YZg),(Nbi,Cj,YZh),(Nbi,Cj,YZi),(Nbi,Cj,YZj),(Nbi,Cj,YZk),(Nbi,Cj ,YZl),(Nbi,Cj,YZm),(Nbi,Cj,YZn),(Nbi,Cj,YZo),(Nbi,Cj,YZp),(Nbi,Cj,YZq),(Nbi,Cj,YZr),( Nbi,Cj,YZs),(Nbi,Cj,YZt),(Nbi,q,YZu),(Nbi,Cj,YZv),(Nbi,Cj,YZw),(Nbi,Cj,YZx),(Nbi,Cj, YZy),(Nbi,Cj,YZz),(Nbi,Cj,YZaa),(Nbi,Cj,YZab),(Nbi,Cj,YZac),(Nbi,Cj,YZad),(Nbi,Cj,YZa e),(Nbi,Cj,YZaf),(Nbi,Cj,YZag),(Nbj,Ca,YZa),(Nbj,Ca,YZb),(Nbj,Ca,YZc),(Nbj,Ca,YZd),(N bj,Ca,YZe),(Nbj,Ca,YZf),(Nbj,Ca,YZg),(Nbj,Ca,YZh),(Nbj,Ca,YZi),(Nbj,Ca,YZj),(Nbj,Ca,Y Zk),(Nbj,Ca,YZl),(Nbj,Ca,YZm),(Nbj,Ca,YZn),(Nbj,Ca,YZo),(Nbj,Ca,YZp),(Nbj,Ca,YZq),( Nbj,Ca,YZr),(Nbj,Ca,YZs),(Nbj,Ca,YZt),(Nbj,Ca,YZu),(Nbj,Ca,YZv),(Nbj,Ca,YZw),(Nbj,C a,YZx),(Nbj,Ca,YZy),(Nbj,Ca,YZz),(Nbj,Ca,YZaa),(Nbj,Ca,YZab),(Nbj,Ca,YZac),(Nbj,Ca, YZad),(Nbj,Ca,YZae),(Nbj,Ca,YZaf),(Nbj,Ca,YZag),(Nbj,Cb,YZa),(Nbj,Cb,YZb),(Nbj,Cb,Y Zc),(Nbj,Cb,YZd),(Nbj,Cb,YZe),(Nbj,Cb,YZf),(Nbj,Cb,YZg),(Nbj,Cb,YZh),(Nbj,Cb,YZi),( Nbj,Cb,YZj),(Nbj,Cb,YZk),(Nbj,Cb,YZl),(Nbj,Cb,YZm),(Nbj,Cb,YZn),(Nbj,Cb,YZo),(Nbj, Cb,YZp),(Nbj,Cb,YZq),(Nbj,Cb,YZr),(Nbj,Cb,YZs),(Nbj,Cb,YZt),(Nbj,Cb,YZu),(Nbj,Cb,Y Zv),(Nbj,Cb,YZw),(Nbj,Cb,YZx),(Nbj,Cb,YZy),(Nbj,Cb,YZz),(Nbj,Cb,YZaa),(Nbj,Cb,YZab) ,(Nbj,Cb,YZac),(Nbj,Cb,YZad),(Nbj,Cb,YZae),(Nbj,Cb,YZaf),(Nbj,Cb,YZag),(Nbj,Cc,YZa) ,(Nbj,Cc,YZb),(Nbj,Cc,YZc),(Nbj,Cc,YZd),(Nbj,Cc,YZe),(Nbj,Cc,YZf),(Nbj,Cc,YZg),(Nbj, Cc,YZh),(Nbj,Cc,YZi),(Nbj,Cc,YZj),(Nbj,Cc,YZk),(Nbj,Cc,YZl),(Nbj,Cc,YZm),(Nbj,Cc,YZ n),(Nbj,Cc,YZo),(Nbj,Cc,YZp),(Nbj,Cc,YZq),(Nbj,Cc,YZr),(Nbj,Cc,YZs),(Nbj,Cc,YZt),(Nbj ,Cc,YZu),(Nbj,Cc,YZv),(Nbj,Cc,YZw),(Nbj,Cc,YZx),(Nbj,Cc,YZy),(Nbj,Cc,YZz),(Nbj,Cc, YZaa),(Nbj,Cc,YZab),(Nbj,Cc,YZac),(Nbj,Cc,YZad),(Nbj,Cc,YZae),(Nbj,Cc,YZaf),(Nbj,Cc, YZag),(Nbj,Cd,YZa),(Nbj,Cd,YZb),(Nbj,Cd,YZc),(Nbj,Cd,YZd),(Nbj,Cd,YZe),(Nbj,Cd,YZf) ,(Nbj,Cd,YZg),(Nbj,Cd,YZh),(Nbj,Cd,YZi),(Nbj,Cd,YZj),(Nbj,Cd,YZk),(Nbj,Cd,YZl),(Nbj, Cd,YZm),(Nbj,Cd,YZn),(Nbj,Cd,YZo),(Nbj,Cd,YZp),(Nbj,Cd,YZq),(Nbj,Cd,YZr),(Nbj,Cd, YZs),(Nbj,Cd,YZt),(Nbj,Cd,YZu),(Nbj,Cd,YZv),(Nbj,Cd,YZw),(Nbj,Cd,YZx),(Nbj,Cd,YZy) ,(Nbj,Cd,YZz),(Nbj,Cd,YZaa),(Nbj,Cd,YZab),(Nbj,Cd,YZac),(Nbj,Cd,YZad),(Nbj,Cd,YZae) ,(Nbj,Cd,YZaf),(Nbj,Cd,YZag),(Nbj,Ce,YZa),(Nbj,Ce,YZb),(Nbj,Ce,YZc),(Nbj,Ce,YZd),(N bj,Ce,YZe),(Nbj,Ce,YZf),(Nbj,Ce,YZg),(Nbj,Ce,YZh),(Nbj,Ce,YZi),(Nbj,Ce,YZj),(Nbj,Ce,Y Zk),(Nbj,Ce,YZl),(Nbj,Ce,YZm),(Nbj,Ce,YZn),(Nbj,Ce,YZo),(Nbj,Ce,YZp),(Nbj,Ce,YZq),( Nbj,Ce,YZr),(Nbj,Ce,YZs),(Nbj,Ce,YZt),(Nbj,Ce,YZu),(Nbj,Ce,YZv),(Nbj,Ce,YZw),(Nbj,C e,YZx),(Nbj,Ce,YZy),(Nbj,Ce,YZz),(Nbj,Ce,YZaa),(Nbj,Ce,YZab),(Nbj,Ce,YZac),(Nbj,Ce, YZad),(Nbj,Ce,YZae),(Nbj,Ce,YZaf),(Nbj,Ce,YZag),(Nbj,Cf,YZa),(Nbj,Cf,YZb),(Nbj,Cf,Y Zc),(Nbj,Cf,YZd),(Nbj,Cf,YZe),(Nbj,Cf,YZf),(Nbj,Cf,YZg),(Nbj,Cf,YZh),(Nbj,Cf,YZi),(Nbj ,Cf,YZj),(Nbj,Cf,YZk),(Nbj,Cf,YZl),(Nbj,Cf,YZm),(Nbj,Cf,YZn),(Nbj,Cf,YZo),(Nbj,Cf,YZ p),(Nbj,Cf,YZq),(Nbj,Cf,YZr),(Nbj,Cf,YZs),(Nbj,Cf,YZt),(Nbj,Cf,YZu),(Nbj,Cf,YZv),(Nbj, Cf,YZw),(Nbj,Cf,YZx),(Nbj,Cf,YZy),(Nbj,Cf,YZz),(Nbj,Cf,YZaa),(Nbj,Cf,YZab),(Nbj,Cf,Y Zac),(Nbj,Cf,YZad),(Nbj,Cf,YZae),(Nbj,Cf,YZaf),(Nbj,Cf,YZag),(Nbj,Cg,YZa),(Nbj,Cg,YZ b),(Nbj,Cg,YZc),(Nbj,Cg,YZd),(Nbj,Cg,YZe),(Nbj,Cg,YZf),(Nbj,Cg,YZg),(Nbj,Cg,YZh),(N bj,Cg,YZi),(Nbj,Cg,YZj),(Nbj,Cg,YZk),(Nbj,Cg,YZl),(Nbj,Cg,YZm),(Nbj,Cg,YZn),(Nbj,Cg, YZo),(Nbj,Cg,YZp),(Nbj,Cg,YZq),(Nbj,Cg,YZr),(Nbj,Cg,YZs),(Nbj,Cg,YZt),(Nbj,Cg,YZu), (Nbj,Cg,YZv),(Nbj,Cg,YZw),(Nbj,Cg,YZx),(Nbj,Cg,YZy),(Nbj,Cg,YZz),(Nbj,Cg,YZaa),(Nb j,Cg,YZab),(Nbj,Cg,YZac),(Nbj,Cg,YZad),(Nbj,Cg,YZae),(Nbj,Cg,YZaf),(Nbj,Cg,YZag),(N bj,Ch,YZa),(Nbj,Ch,YZb),(Nbj,Ch,YZc),(Nbj,Ch,YZd),(Nbj,Ch,YZe),(Nbj,Ch,YZf),(Nbj,Ch, YZg),(Nbj,Ch,YZh),(Nbj,Ch,YZi),(Nbj,Ch,YZj),(Nbj,Ch,YZk),(Nbj,Ch,YZl),(Nbj,Ch,YZm), (Nbj,Ch,YZn),(Nbj,Ch,YZo),(Nbj,Ch,YZp),(Nbj,Ch,YZq),(Nbj,Ch,YZr),(Nbj,Ch,YZs),(Nbj, Ch,YZt),(Nbj,Ch,YZu),(Nbj,Ch,YZv),(Nbj,Ch,YZw),(Nbj,Ch,YZx),(Nbj,Ch,YZy),(Nbj,Ch, YZz),(Nbj,Ch,YZaa),(Nbj,Ch,YZab),(Nbj,Ch,YZac),(Nbj,Ch,YZad),(Nbj,Ch,YZae),(Nbj,Ch, YZaf),(Nbj,Ch,YZag),(Nbj,Ci,YZa),(Nbj,Ci,YZb),(Nbj,Ci,YZc),(Nbj,Ci,YZd),(Nbj,Ci,YZe), (Nbj,Ci,YZf),(Nbj,Ci,YZg),(Nbj,Ci,YZh),(Nbj,Ci,YZi),(Nbj,Ci,YZj),(Nbj,Ci,YZk),(Nbj,Ci,Y Zl),(Nbj,Ci,YZm),(Nbj,Ci,YZn),(Nbj,C i,YZo),(Nbj,Ci,YZp),(Nbj,Ci,YZq),(Nbj,C i,YZr),(Nbj ,Ci,YZs),(Nbj,Ci,YZt),(Nbj,Ci,YZu),(Nbj,Ci,YZv),(Nbj,Ci,YZw),(Nbj,Ci,YZx),(Nbj,Ci,YZy) ,(Nbj,Ci,YZz),(Nbj,Ci,YZaa),(Nbj,Ci,YZab),(Nbj,Ci,YZac),(Nbj,Ci,YZad),(Nbj,Ci,YZae),( Nbj,Ci,YZaf),(Nbj,Ci,YZag),(Nbj,Cj,YZa),(Nbj,Cj,YZb),(Nbj,Cj,YZc),(Nbj,Cj,YZd),(Nbj,Cj ,YZe),(Nbj,Cj,YZf),(Nbj,Cj,YZg),(Nbj,Cj,YZh),(Nbj,Cj,YZi),(Nbj,Cj,YZj),(Nbj,Cj,YZk),(N bj,Cj,YZl),(Nbj,Cj,YZm),(Nbj,Cj,YZn),(Nbj,Cj,YZo),(Nbj,Cj,YZp),(Nbj,Cj,YZq),(Nbj,Cj,Y Zr),(Nbj,Cj,YZs),(Nbj,Cj,YZt),(Nbj,Cj,YZu),(Nbj,Cj,YZv),(Nbj,Cj,YZw),(Nbj,Cj,YZx),(Nbj ,Cj,YZy),(Nbj,Cj,YZz),(Nbj,Cj,YZaa),(Nbj,Cj,YZab),(Nbj,Cj,YZac),(Nbj,Cj,YZad),(Nbj,Cj, YZae),(Nbj,Cj,YZaf),(Nbj,Cj,YZag),(Nbk,Ca,YZa),(Nbk,Ca,YZb),(Nbk,Ca,YZc),(Nbk,Ca,Y Zd),(Nbk,Ca,YZe),(Nbk,Ca,YZf),(Nbk,Ca,YZg),(Nbk,Ca,YZh),(Nbk,Ca,YZi),(Nbk,Ca,YZj), (Nbk,Ca,YZk),(Nbk,Ca,YZl),(Nbk,Ca,YZm),(Nbk,Ca,YZn),(Nbk,Ca,YZo),(Nbk,Ca,YZp),(N bk,Ca,YZq),(Nbk,Ca,YZr),(Nbk,Ca,YZs),(Nbk,Ca,YZt),(Nbk,Ca,YZu),(Nbk,Ca,YZv),(Nbk, Ca,YZw),(Nbk,Ca,YZx),(Nbk,Ca,YZy),(Nbk,Ca,YZz),(Nbk,Ca,YZaa),(Nbk,Ca,YZab),(Nbk, Ca,YZac),(Nbk,Ca,YZad),(Nbk,Ca,YZae),(Nbk,Ca,YZaf),(Nbk,Ca,YZag),(Nbk,Cb,YZa),(N bk,Cb,YZb),(Nbk,Cb,YZc),(Nbk,Cb,YZd),(Nbk,Cb,YZe),(Nbk,Cb,YZf),(Nbk,Cb,YZg),(Nbk ,Cb,YZh),(Nbk,Cb,YZi),(Nbk,Cb,YZj),(Nbk,Cb,YZk),(Nbk,Cb,YZl),(Nbk,Cb,YZm),(Nbk,C b,YZn),(Nbk,Cb,YZo),(Nbk,Cb,YZp),(Nbk,Cb,YZq),(Nbk,Cb,YZr),(Nbk,Cb,YZs),(Nbk,Cb, YZt),(Nbk,Cb,YZu),(Nbk,Cb,YZv),(Nbk,Cb,YZw),(Nbk,Cb,YZx),(Nbk,Cb,YZy),(Nbk,Cb,Y Zz),(Nbk,Cb,YZaa),(Nbk,Cb,YZab),(Nbk,Cb,YZac),(Nbk,Cb,YZad),(Nbk,Cb,YZae),(Nbk,C b,YZaf),(Nbk,Cb,YZag),(Nbk,Cc,YZa),(Nbk,Cc,YZb),(Nbk,Cc,YZc),(Nbk,Cc,YZd),(Nbk,C c,YZe),(Nbk,Cc,YZf),(Nbk,Cc,YZg),(Nbk,Cc,YZh),(Nbk,Cc,YZi),(Nbk,Cc,YZj),(Nbk,Cc,Y Zk),(Nbk,Cc,YZl),(Nbk,Cc,YZm),(Nbk,Cc,YZn),(Nbk,Cc,YZo),(Nbk,Cc,YZp),(Nbk,Cc,YZ q),(Nbk,Cc,YZr),(Nbk,Cc,YZs),(Nbk,Cc,YZt),(Nbk,Cc,YZu),(Nbk,Cc,YZv),(Nbk,Cc,YZw), (Nbk,Cc,YZx),(Nbk,Cc,YZy),(Nbk,Cc,YZz),(Nbk,Cc,YZaa),(Nbk,Cc,YZab),(Nbk,Cc,YZac) ,(Nbk,Cc,YZad),(Nbk,Cc,YZae),(Nbk,Cc,YZaf),(Nbk,Cc,YZag),(Nbk,Cd,YZa),(Nbk,Cd,YZ b),(Nbk,Cd,YZc),(Nbk,Cd,YZd),(Nbk,Cd,YZe),(Nbk,Cd,YZf),(Nbk,Cd,YZg),(Nbk,Cd,YZh) ,(Nbk,Cd,YZi),(Nbk,Cd,YZj),(Nbk,Cd,YZk),(Nbk,Cd,YZl),(Nbk,Cd,YZm),(Nbk,Cd,YZn),( Nbk,Cd,YZo),(Nbk,Cd,YZp),(Nbk,Cd,YZq),(Nbk,Cd,YZr),(Nbk,Cd,YZs),(Nbk,Cd,YZt),(Nb k,Cd,YZu),(Nbk,Cd,YZv),(Nbk,Cd,YZw),(Nbk,Cd,YZx),(Nbk,Cd,YZy),(Nbk,Cd,YZz),(Nbk ,Cd,YZaa),(Nbk,Cd,YZab),(Nbk,Cd,YZac),(Nbk,Cd,YZad),(Nbk,Cd,YZae),(Nbk,Cd,YZaf),( Nbk,Cd,YZag),(Nbk,Ce,YZa),(Nbk,Ce,YZb),(Nbk,Ce,YZc),(Nbk,Ce,YZd),(Nbk,Ce,YZe),(N bk,Ce,YZf),(Nbk,Ce,YZg),(Nbk,Ce,YZh),(Nbk,Ce,YZi),(Nbk,Ce,YZj),(Nbk,Ce,YZk),(Nbk, Ce,YZl),(Nbk,Ce,YZm),(Nbk,Ce,YZn),(Nbk,Ce,YZo),(Nbk,Ce,YZp),(Nbk,Ce,YZq),(Nbk,C e,YZr),(Nbk,Ce,YZs),(Nbk,Ce,YZt),(Nbk,Ce,YZu),(Nbk,Ce,YZv),(Nbk,Ce,YZw),(Nbk,Ce, YZx),(Nbk,Ce,YZy),(Nbk,Ce,YZz),(Nbk,Ce,YZaa),(Nbk,Ce,YZab),(Nbk,Ce,YZac),(Nbk,Ce ,YZad),(Nbk,Ce,YZae),(Nbk,Ce,YZaf),(Nbk,Ce,YZag),(Nbk,Cf,YZa),(Nbk,Cf,YZb),(Nbk,C f,YZc),(Nbk,Cf,YZd),(Nbk,Cf,YZe),(Nbk,Cf,YZf),(Nbk,Cf,YZg),(Nbk,Cf,YZh),(Nbk,Cf,YZ i),(Nbk,Cf,YZj),(Nbk,Cf,YZk),(Nbk,Cf,YZl),(Nbk,Cf,YZm),(Nbk,Cf,YZn),(Nbk,Cf,YZo),(N bk,Cf,YZp),(Nbk,Cf,YZq),(Nbk,Cf,YZr),(Nbk,Cf,YZs),(Nbk,Cf,YZt),(Nbk,Cf,YZu),(Nbk,Cf ,YZv),(Nbk,Cf,YZw),(Nbk,Cf,YZx),(Nbk,Cf,YZy),(Nbk,Cf,YZz),(Nbk,Cf,YZaa),(Nbk,Cf,Y Zab),(Nbk,Cf,YZac),(Nbk,Cf,YZad),(Nbk,Cf,YZae),(Nbk,Cf,YZaf),(Nbk,Cf,YZag),(Nbk,Cg ,YZa),(Nbk,Cg,YZb),(Nbk,Cg,YZc),(Nbk,Cg,YZd),(Nbk,Cg,YZe),(Nbk,Cg,YZf),(Nbk,Cg,Y Zg),(Nbk,Cg,YZh),(Nbk,Cg,YZi),(Nbk,Cg,YZj),(Nbk,Cg,YZk),(Nbk,Cg,YZl),(Nbk,Cg,YZm) ,(Nbk,Cg,YZn),(Nbk,Cg,YZo),(Nbk,Cg,YZp),(Nbk,Cg,YZq),(Nbk,Cg,YZr),(Nbk,Cg,YZs),( Nbk,Cg,YZt),(Nbk,Cg,YZu),(Nbk,Cg,YZv),(Nbk,Cg,YZw),(Nbk,Cg,YZx),(Nbk,Cg,YZy),(N bk,Cg,YZz),(Nbk,Cg,YZaa),(Nbk,Cg,YZab),(Nbk,Cg,YZac),(Nbk,Cg,YZad),(Nbk,Cg,YZae) ,(Nbk,Cg,YZaf),(Nbk,Cg,YZag),(Nbk,Ch,YZa),(Nbk,Ch,YZb),(Nbk,Ch,YZc),(Nbk,Ch,YZd) ,(Nbk,Ch,YZe),(Nbk,Ch,YZf),(Nbk,Ch,YZg),(Nbk,Ch,YZh),(Nbk,Ch,YZi),(Nbk,Ch,YZj),(N bk,Ch,YZk),(Nbk,Ch,YZl),(Nbk,Ch,YZm),(Nbk,Ch,YZn),(Nbk,Ch,YZo),(Nbk,Ch,YZp),(Nb k,Ch,YZq),(Nbk,Ch,YZr),(Nbk,Ch,YZs),(Nbk,Ch,YZt),(Nbk,Ch,YZu),(Nbk,Ch,YZv),(Nbk, Ch,YZw),(Nbk,Ch,YZx),(Nbk,Ch,YZy),(Nbk,Ch,YZz),(Nbk,Ch,YZaa),(Nbk,Ch,YZab),(Nbk ,Ch,YZac),(Nbk,Ch,YZad),(Nbk,Ch,YZae),(Nbk,Ch,YZaf),(Nbk,Ch,YZag),(Nbk,Ci,YZa),(N bk,Ci,YZb),(Nbk,Ci,YZc),(Nbk,Ci,YZd),(Nbk,Ci,YZe),(Nbk,Ci,YZf),(Nbk,Ci,YZg),(Nbk,Ci ,YZh),(Nbk,Ci,YZi),(Nbk,Ci,YZj),(Nbk,Ci,YZk),(Nbk,Ci,YZl),(Nbk,Ci,YZm),(Nbk,Ci,YZn) ,(Nbk,Ci,YZo),(Nbk,Ci,YZp),(Nbk,Ci,YZq),(Nbk,Ci,YZr),(Nbk,Ci,YZs),(Nbk,Ci,YZt),(Nbk, Ci,YZu),(Nbk,Ci,YZv),(Nbk,Ci,YZw),(Nbk,Ci,YZx),(Nbk,Ci,YZy),(Nbk,Ci,YZz),(Nbk,Ci,Y Zaa),(Nbk,Ci,YZab),(Nbk,Ci,YZac),(Nbk,Ci,YZad),(Nbk,Ci,YZae),(Nbk,Ci,YZaf),(Nbk,Ci, YZag),(Nbk,Cj,YZa),(Nbk,Cj,YZb),(Nbk,Cj,YZc),(Nbk,Cj,YZd),(Nbk,Cj,YZe),(Nbk,Cj,YZf) ,(Nbk,Cj,YZg),(Nbk,Cj,YZh),(Nbk,Cj,YZi),(Nbk,Cj,YZj),(Nbk,Cj,YZk),(Nbk,Cj,YZl),(Nbk ,Cj,YZm),(Nbk,Cj,YZn),(Nbk,Cj,YZo),(Nbk,Cj,YZp),(Nbk,Cj,YZq),(Nbk,Cj,YZr),(Nbk,Cj, YZs),(Nbk,Cj,YZt),(Nbk,Cj,YZu),(Nbk,Cj,YZv),(Nbk,Cj,YZw),(Nbk,Cj,YZx),(Nbk,Cj,YZy) ,(Nbk,Cj,YZz),(Nbk,Cj,YZaa),(Nbk,Cj,YZab),(Nbk,Cj,YZac),(Nbk,Cj,YZad),(Nbk,Cj,YZae) ,(Nbk,Cj,YZaf),(Nbk,Cj,YZag),(Nbl,Ca,YZa),(Nbl,Ca,YZb),(Nbl,Ca,YZc),(Nbl,Ca,YZd),(N bl,Ca,YZe),(Nbl,Ca,YZf),(Nbl,Ca,YZg),(Nbl,Ca,YZh),(Nbl,Ca,YZi),(Nbl,Ca,YZj),(Nbl,Ca,Y Zk),(Nbl,Ca,YZl),(Nbl,Ca,YZm),(Nbl,Ca,YZn),(Nbl,Ca,YZo),(Nbl,Ca,YZp),(Nbl,Ca,YZq),( Nbl,Ca,YZr),(Nbl,Ca,YZs),(Nbl,Ca,YZt),(Nbl,Ca,YZu),(Nbl,Ca,YZv),(Nbl,Ca,YZw),(Nbl,C a,YZx),(Nbl,Ca,YZy),(Nbl,Ca,YZz),(Nbl,Ca,YZaa),(Nbl,Ca,YZab),(Nbl,Ca,YZac),(Nbl,Ca, YZad),(Nbl,Ca,YZae),(Nbl,Ca,YZaf),(Nbl,Ca,YZag),(Nbl,Cb,YZa),(Nbl,Cb,YZb),(Nbl,Cb,Y Zc),(Nbl,Cb,YZd),(Nbl,Cb,YZe),(Nbl,Cb,YZf),(Nbl,Cb,YZg),(Nbl,Cb,YZh),(Nbl,Cb,YZi),( Nbl,Cb,YZj),(Nbl,Cb,YZk),(Nbl,Cb,YZl),(Nbl,Cb,YZm),(Nbl,Cb,YZn),(Nbl,Cb,YZo),(Nbl, Cb,YZp),(Nbl,Cb,YZq),(Nbl,Cb,YZr),(Nbl,Cb,YZs),(Nbl,Cb,YZt),(Nbl,Cb,YZu),(Nbl,Cb,Y Zv),(Nbl,Cb,YZw),(Nbl,Cb,YZx),(Nbl,Cb,YZy),(Nbl,Cb,YZz),(Nbl,Cb,YZaa),(Nbl,Cb,YZab) ,(Nbl,Cb,YZac),(Nbl,Cb,YZad),(Nbl,Cb,YZae),(Nbl,Cb,YZaf),(Nbl,Cb,YZag),(Nbl,Cc,YZa) ,(Nbl,Cc,YZb),(Nbl,Cc,YZc),(Nbl,Cc,YZd),(Nbl,Cc,YZe),(Nbl,Cc,YZf),(Nbl,Cc,YZg),(Nbl, Cc,YZh),(Nbl,Cc,YZi),(Nbl,Cc,YZj),(Nbl,Cc,YZk),(Nbl,Cc,YZl),(Nbl,Cc,YZm),(Nbl,Cc,YZ n),(Nbl,Cc,YZo),(Nbl,Cc,YZp),(Nbl,Cc,YZq),(Nbl,Cc,YZr),(Nbl,Cc,YZs),(Nbl,Cc,YZt),(Nbl ,Cc,YZu),(Nbl,Cc,YZv),(Nbl,Cc,YZw),(Nbl,Cc,YZx),(Nbl,Cc,YZy),(Nbl,Cc,YZz),(Nbl,Cc, YZaa),(Nbl,Cc,YZab),(Nbl,Cc,YZac),(Nbl,Cc,YZad),(Nbl,Cc,YZae),(Nbl,Cc,YZaf),(Nbl,Cc, YZag),(Nbl,Cd,YZa),(Nbl,Cd,YZb),(Nbl,Cd,YZc),(Nbl,Cd,YZd),(Nbl,Cd,YZe),(Nbl,Cd,YZf) ,(Nbl,Cd,YZg),(Nbl,Cd,YZh),(Nbl,Cd,YZi),(Nbl,Cd,YZj),(Nbl,Cd,YZk),(Nbl,Cd,YZl),(Nbl, Cd,YZm),(Nbl,Cd,YZn),(Nbl,Cd,YZo),(Nbl,Cd,YZp),(Nbl,Cd,YZq),(Nbl,Cd,YZr),(Nbl,Cd, YZs),(Nbl,Cd,YZt),(Nbl,Cd,YZu),(Nbl,Cd,YZv),(Nbl,Cd,YZw),(Nbl,Cd,YZx),(Nbl,Cd,YZy) ,(Nbl,Cd,YZz),(Nbl,Cd,YZaa),(Nbl,Cd,YZab),(Nbl,Cd,YZac),(Nbl,Cd,YZad),(Nbl,Cd,YZae) ,(Nbl,Cd,YZaf),(Nbl,Cd,YZag),(Nbl,Ce,YZa),(Nbl,Ce,YZb),(Nbl,Ce,YZc),(Nbl,Ce,YZd),(N bl,Ce,YZe),(Nbl,Ce,YZf),(Nbl,Ce,YZg),(Nbl,Ce,YZh),(Nbl,Ce,YZi),(Nbl,Ce,YZj),(Nbl,Ce,Y Zk),(Nbl,Ce,YZl),(Nbl,Ce,YZm),(Nbl,Ce,YZn),(Nbl,Ce,YZo),(Nbl,Ce,YZp),(Nbl,Ce,YZq),( Nbl,Ce,YZr),(Nbl,Ce,YZs),(Nbl,Ce,YZt),(Nbl,Ce,YZu),(Nbl,Ce,YZv),(Nbl,Ce,YZw),(Nbl,C e,YZx),(Nbl,Ce,YZy),(Nbl,Ce,YZz),(Nbl,Ce,YZaa),(Nbl,Ce,YZab),(Nbl,Ce,YZac),(Nbl,Ce, YZad),(Nbl,Ce,YZae),(Nbl,Ce,YZaf),(Nbl,Ce,YZag),(Nbl,Cf,YZa),(Nbl,Cf,YZb),(Nbl,Cf,Y Zc),(Nbl,Cf,YZd),(Nbl,Cf,YZe),(Nbl,Cf,YZf),(Nbl,Cf,YZg),(Nbl,Cf,YZh),(Nbl,Cf,YZi),(Nbl ,Cf,YZj),(Nbl,Cf,YZk),(Nbl,Cf,YZl),(Nbl,Cf,YZm),(Nbl,Cf,YZn),(Nbl,Cf,YZo),(Nbl,Cf,YZ p),(Nbl,Cf,YZq),(Nbl,Cf,YZr),(Nbl,Cf,YZs),(Nbl,Cf,YZt),(Nbl,Cf,YZu),(Nbl,Cf,YZv),(Nbl, Cf,YZw),(Nbl,Cf,YZx),(Nbl,Cf,YZy),(Nbl,Cf,YZz),(Nbl,Cf,YZaa),(Nbl,Cf,YZab),(Nbl,Cf,Y Zac),(Nbl,Cf,YZad),(Nbl,Cf,YZae),(Nbl,Cf,YZaf),(Nbl,Cf,YZag),(Nbl,Cg,YZa),(Nbl,Cg,YZ b),(Nbl,Cg,YZc),(Nbl,Cg,YZd),(Nbl,Cg,YZe),(Nbl,Cg,YZf),(Nbl,Cg,YZg),(Nbl,Cg,YZh),(N bl,Cg,YZi),(Nbl,Cg,YZj),(Nbl,Cg,YZk),(Nbl,Cg,YZl),(Nbl,Cg,YZm),(Nbl,Cg,YZn),(Nbl,Cg, YZo),(Nbl,Cg,YZp),(Nbl,Cg,YZq),(Nbl,Cg,YZr),(Nbl,Cg,YZs),(Nbl,Cg,YZt),(Nbl,Cg,YZu), (Nbl,Cg,YZv),(Nbl,Cg,YZw),(Nbl,Cg,YZx),(Nbl,Cg,YZy),(Nbl,Cg,YZz),(Nbl,Cg,YZaa),(Nb l,Cg,YZab),(Nbl,Cg,YZac),(Nbl,Cg,YZad),(Nbl,Cg,YZae),(Nbl,Cg,YZaf),(Nbl,Cg,YZag),(N bl,Ch,YZa),(Nbl,Ch,YZb),(Nbl,Ch,YZc),(Nbl,Ch,YZd),(Nbl,Ch,YZe),(Nbl,Ch,YZf),(Nbl,Ch, YZg),(Nbl,Ch,YZh),(Nbl,Ch,YZi),(Nbl,Ch,YZj),(Nbl,Ch,YZk),(Nbl,Ch,YZl),(Nbl,Ch,YZm), (Nbl,Ch,YZn),(Nbl,Ch,YZo),(Nbl,Ch,YZp),(Nbl,Ch,YZq),(Nbl,Ch,YZr),(Nbl,Ch,YZs),(Nbl, Ch,YZt),(Nbl,Ch,YZu),(Nbl,Ch,YZv),(Nbl,Ch,YZw),(Nbl,Ch,YZx),(Nbl,Ch,YZy),(Nbl,Ch, YZz),(Nbl,Ch,YZaa),(Nbl,Ch,YZab),(Nbl,Ch,YZac),(Nbl,Ch,YZad),(Nbl,Ch,YZae),(Nbl,Ch, YZaf),(Nbl,Ch,YZag),(Nbl,Ci,YZa),(Nbl,Ci,YZb),(Nbl,Ci,YZc),(Nbl,Ci,YZd),(Nbl,Ci,YZe), (Nbl,Ci,YZf),(Nbl,Ci,YZg),(Nbl,Ci,YZh),(Nbl,Ci,YZi),(Nbl,Ci,YZj),(Nbl,Ci,YZk),(Nbl,Ci,Y Zl),(Nbl,Ci,YZm),(Nbl,Ci,YZn),(Nbl,Ci,YZo),(Nbl,Ci,YZp),(Nbl,Ci,YZq),(Nbl,Ci,YZr),(Nbl ,Ci,YZs),(Nbl,Ci,YZt),(Nbl,Ci,YZu),(Nbl,Ci,YZv),(Nbl,Ci,YZw),(Nbl,Ci,YZx),(Nbl,Ci,YZy) ,(Nbl,Ci,YZz),(Nbl,Ci,YZaa),(Nbl,Ci,YZab),(Nbl,Ci,YZac),(Nbl,Ci,YZad),(Nbl,Ci,YZae),( Nbl,Ci,YZaf),(Nbl,Ci,YZag),(Nbl,Cj,YZa),(Nbl,Cj,YZb),(Nbl,Cj,YZc),(Nbl,Cj,YZd),(Nbl,Cj ,YZe),(Nbl,Cj,YZf),(Nbl,Cj,YZg),(Nbl,Cj,YZh),(Nbl,Cj,YZi),(Nbl,Cj,YZj),(Nbl,Cj,YZk),(N bl,Cj,YZl),(Nbl,Cj,YZm),(Nbl,Cj,YZn),(Nbl,Cj,YZo),(Nbl,Cj,YZp),(Nbl,Cj,YZq),(Nbl,Cj,Y Zr),(Nbl,Cj,YZs),(Nbl,Cj,YZt),(Nbl,Cj,YZu),(Nbl,Cj,YZv),(Nbl,Cj,YZw),(Nbl,Cj,YZx),(Nbl ,Cj,YZy),(Nbl,Cj,YZz),(Nbl,Cj,YZaa),(Nbl,Cj,YZab),(Nbl,Cj,YZac),(Nbl,Cj,YZad),(Nbl,Cj, YZae),(Nbl,Cj,YZaf),(Nbl,Cj,YZag),(Nbm,Ca,YZa),(Nbm,Ca,YZb),(Nbm,Ca,YZc),(Nbm,Ca ,YZd),(Nbm,Ca,YZe),(Nbm,Ca,YZf),(Nbm,Ca,YZg),(Nbm,Ca,YZh),(Nbm,Ca,YZi),(Nbm,C a,YZj),(Nbm,Ca,YZk),(Nbm,Ca,YZl),(Nbm,Ca,YZm),(Nbm,Ca,YZn),(Nbm,Ca,YZo),(Nbm, Ca,YZp),(Nbm,Ca,YZq),(Nbm,Ca,YZr),(Nbm,Ca,YZs),(Nbm,Ca,YZt),(Nbm,Ca,YZu),(Nbm ,Ca,YZv),(Nbm,Ca,YZw),(Nbm,Ca,YZx),(Nbm,Ca,YZy),(Nbm,Ca,YZz),(Nbm,Ca,YZaa),(N bm,Ca,YZab),(Nbm,Ca,YZac),(Nbm,Ca,YZad),(Nbm,Ca,YZae),(Nbm,Ca,YZaf),(Nbm,Ca,Y Zag),(Nbm,Cb,YZa),(Nbm,Cb,YZb),(Nbm,Cb,YZc),(Nbm,Cb,YZd),(Nbm,Cb,YZe),(Nbm,C b,YZf),(Nbm,Cb,YZg),(Nbm,Cb,YZh),(Nbm,Cb,YZi),(Nbm,Cb,YZj),(Nbm,Cb,YZk),(Nbm, Cb,YZl),(Nbm,Cb,YZm),(Nbm,Cb,YZn),(Nbm,Cb,YZo),(Nbm,Cb,YZp),(Nbm,Cb,YZq),(Nb m,Cb,YZr),(Nbm,Cb,YZs),(Nbm,Cb,YZt),(Nbm,Cb,YZu),(Nbm,Cb,YZv),(Nbm,Cb,YZw),( Nbm,Cb,YZx),(Nbm,Cb,YZy),(Nbm,Cb,YZz),(Nbm,Cb,YZaa),(Nbm,Cb,YZab),(Nbm,Cb,Y Zac),(Nbm,Cb,YZad),(Nbm,Cb,YZae),(Nbm,Cb,YZaf),(Nbm,Cb,YZag),(Nbm,Cc,YZa),(Nb m,Cc,YZb),(Nbm,Cc,YZc),(Nbm,Cc,YZd),(Nbm,Cc,YZe),(Nbm,Cc,YZf),(Nbm,Cc,YZg),(N bm,Cc,YZh),(Nbm,Cc,YZi),(Nbm,Cc,YZj),(Nbm,Cc,YZk),(Nbm,Cc,YZl),(Nbm,Cc,YZm),( Nbm,Cc,YZn),(Nbm,Cc,YZo),(Nbm,Cc,YZp),(Nbm,Cc,YZq),(Nbm,Cc,YZr),(Nbm,Cc,YZs), (Nbm,Cc,YZt),(Nbm,Cc,YZu),(Nbm,Cc,YZv),(Nbm,Cc,YZw),(Nbm,Cc,YZx),(Nbm,Cc,YZy) ,(Nbm,Cc,YZz),(Nbm,Cc,YZaa),(Nbm,Cc,YZab),(Nbm,Cc,YZac),(Nbm,Cc,YZad),(Nbm,C c,YZae),(Nbm,Cc,YZaf),(Nbm,Cc,YZag),(Nbm,Cd,YZa),(Nbm,Cd,YZb),(Nbm,Cd,YZc),(N bm,Cd,YZd),(Nbm,Cd,YZe),(Nbm,Cd,YZf),(Nbm,Cd,YZg),(Nbm,Cd,YZh),(Nbm,Cd,YZi),( Nbm,Cd,YZj),(Nbm,Cd,YZk),(Nbm,Cd,YZl),(Nbm,Cd,YZm),(Nbm,Cd,YZn),(Nbm,Cd,YZo) ,(Nbm,Cd,YZp),(Nbm,Cd,YZq),(Nbm,Cd,YZr),(Nbm,Cd,YZs),(Nbm,Cd,YZt),(Nbm,Cd,YZ u),(Nbm,Cd,YZv),(Nbm,Cd,YZw),(Nbm,Cd,YZx),(Nbm,Cd,YZy),(Nbm,Cd,YZz),(Nbm,Cd, YZaa),(Nbm,Cd,YZab),(Nbm,Cd,YZac),(Nbm,Cd,YZad),(Nbm,Cd,YZae),(Nbm,Cd,YZaf),( Nbm,Cd,YZag),(Nbm,Ce,YZa),(Nbm,Ce,YZb),(Nbm,Ce,YZc),(Nbm,Ce,YZd),(Nbm,Ce,YZe) ,(Nbm,Ce,YZf),(Nbm,Ce,YZg),(Nbm,Ce,YZh),(Nbm,Ce,YZi),(Nbm,Ce,YZj),(Nbm,Ce,YZk) ,(Nbm,Ce,YZl),(Nbm,Ce,YZm),(Nbm,Ce,YZn),(Nbm,Ce,YZo),(Nbm,Ce,YZp),(Nbm,Ce,Y Zq),(Nbm,Ce,YZr),(Nbm,Ce,YZs),(Nbm,Ce,YZt),(Nbm,Ce,YZu),(Nbm,Ce,YZv),(Nbm,Ce, YZw),(Nbm,Ce,YZx),(Nbm,Ce,YZy),(Nbm,Ce,YZz),(Nbm,Ce,YZaa),(Nbm,Ce,YZab),(Nbm ,Ce,YZac),(Nbm,Ce,YZad),(Nbm,Ce,YZae),(Nbm,Ce,YZaf),(Nbm,Ce,YZag),(Nbm,Cf,YZa), (Nbm,Cf,YZb),(Nbm,Cf,YZc),(Nbm,Cf,YZd),(Nbm,Cf,YZe),(Nbm,Cf,YZf),(Nbm,Cf,YZg),( Nbm,Cf,YZh),(Nbm,Cf,YZi),(Nbm,Cf,YZj),(Nbm,Cf,YZk),(Nbm,Cf,YZl),(Nbm,Cf,YZm),( Nbm,Cf,YZn),(Nbm,Cf,YZo),(Nbm,Cf,YZp),(Nbm,Cf,YZq),(Nbm,Cf,YZr),(Nbm,Cf,YZs),( Nbm,Cf,YZt),(Nbm,Cf,YZu),(Nbm,Cf,YZv),(Nbm,Cf,YZw),(Nbm,Cf,YZx),(Nbm,Cf,YZy),( Nbm,Cf,YZz),(Nbm,Cf,YZaa),(Nbm,Cf,YZab),(Nbm,Cf,YZac),(Nbm,Cf,YZad),(Nbm,Cf,Y Zae),(Nbm,Cf,YZaf),(Nbm,Cf,YZag),(Nbm,Cg,YZa),(Nbm,Cg,YZb),(Nbm,Cg,YZc),(Nbm, Cg,YZd),(Nbm,Cg,YZe),(Nbm,Cg,YZf),(Nbm,Cg,YZg),(Nbm,Cg,YZh),(Nbm,Cg,YZi),(Nb m,Cg,YZj),(Nbm,Cg,YZk),(Nbm,Cg,YZl),(Nbm,Cg,YZm),(Nbm,Cg,YZn),(Nbm,Cg,YZo),( Nbm,Cg,YZp),(Nbm,Cg,YZq),(Nbm,Cg,YZr),(Nbm,Cg,YZs),(Nbm,Cg,YZt),(Nbm,Cg,YZu) ,(Nbm,Cg,YZv),(Nbm,Cg,YZw),(Nbm,Cg,YZx),(Nbm,Cg,YZy),(Nbm,Cg,YZz),(Nbm,Cg,Y Zaa),(Nbm,Cg,YZab),(Nbm,Cg,YZac),(Nbm,Cg,YZad),(Nbm,Cg,YZae),(Nbm,Cg,YZaf),(N bm,Cg,YZag),(Nbm,Ch,YZa),(Nbm,Ch,YZb),(Nbm,Ch,YZc),(Nbm,Ch,YZd),(Nbm,Ch,YZe) ,(Nbm,Ch,YZf),(Nbm,Ch,YZg),(Nbm,Ch,YZh),(Nbm,Ch,YZi),(Nbm,Ch,YZj),(Nbm,Ch,YZk) ,(Nbm,Ch,YZl),(Nbm,Ch,YZm),(Nbm,Ch,YZn),(Nbm,Ch,YZo),(Nbm,Ch,YZp),(Nbm,Ch,Y Zq),(Nbm,Ch,YZr),(Nbm,Ch,YZs),(Nbm,Ch,YZt),(Nbm,Ch,YZu),(Nbm,Ch,YZv),(Nbm,Ch, YZw),(Nbm,Ch,YZx),(Nbm,Ch,YZy),(Nbm,Ch,YZz),(Nbm,Ch,YZaa),(Nbm,Ch,YZab),(Nb m,Ch,YZac),(Nbm,Ch,YZad),(Nbm,Ch,YZae),(Nbm,Ch,YZaf),(Nbm,Ch,YZag),(Nbm,Ci,YZ a),(Nbm,Ci,YZb),(Nbm,Ci,YZc),(Nbm,Ci,YZd),(Nbm,Ci,YZe),(Nbm,Ci,YZf),(Nbm,Ci,YZg) ,(Nbm,Ci,YZh),(Nbm,Ci,YZi),(Nbm,Ci,YZj),(Nbm,Ci,YZk),(Nbm,Ci,YZl),(Nbm,Ci,YZm), (Nbm,Ci,YZn),(Nbm,Ci,YZo),(Nbm,Ci,YZp),(Nbm,Ci,YZq),(Nbm,Ci,YZr),(Nbm,Ci,YZs),( Nbm,CLYZt),(Nbm,Ci,YZu),(Nbm,Ci,YZv),(Nbm,Ci,YZw),(Nbm,Ci,YZx),(Nbm,Ci,YZy),( Nbm,Ci,YZz),(Nbm,Ci,YZaa),(Nbm,Ci,YZab),(Nbm,Ci,YZac),(Nbm,Ci,YZad),(Nbm,Ci,YZ ae),(Nbm,Ci,YZaf),(Nbm,Ci,YZag),(Nbm,Cj,YZa),(Nbm,Cj,YZb),(Nbm,Cj,YZc),(Nbm,Cj,Y Zd),(Nbm,Cj,YZe),(Nbm,Cj,YZf),(Nbm,Cj,YZg),(Nbm,Cj,YZh),(Nbm,Cj,YZi),(Nbm,Cj,YZj) ,(Nbm,Cj,YZk),(Nbm,Cj,YZl),(Nbm,Cj,YZm),(Nbm,Cj,YZn),(Nbm,Cj,YZo),(Nbm,Cj,YZp) ,(Nbm,Cj,YZq),(Nbm,Cj,YZr),(Nbm,Cj,YZs),(Nbm,Cj,YZt),(Nbm,Cj,YZu),(Nbm,Cj,YZv),( Nbm,Cj,YZw),(Nbm,Cj,YZx),(Nbm,Cj,YZy),(Nbm,Cj,YZz),(Nbm,Cj,YZaa),(Nbm,Cj,YZab) ,(Nbm,Cj,YZac),(Nbm,Cj,YZad),(Nbm,Cj,YZae),(Nbm,Cj,YZaf),(Nbm,Cj,YZag),(Nbn,Ca,Y Za),(Nbn,Ca,YZb),(Nbn,Ca,YZc),(Nbn,Ca,YZd),(Nbn,Ca,YZe),(Nbn,Ca,YZf),(Nbn,Ca,YZg) ,(Nbn,Ca,YZh),(Nbn,Ca,YZi),(Nbn,Ca,YZj),(Nbn,Ca,YZk),(Nbn,Ca,YZl),(Nbn,Ca,YZm),(N bn,Ca,YZn),(Nbn,Ca,YZo),(Nbn,Ca,YZp),(Nbn,Ca,YZq),(Nbn,Ca,YZr),(Nbn,Ca,YZs),(Nbn, Ca,YZt),(Nbn,Ca,YZu),(Nbn,Ca,YZv),(Nbn,Ca,YZw),(Nbn,Ca,YZx),(Nbn,Ca,YZy),(Nbn,Ca ,YZz),(Nbn,Ca,YZaa),(Nbn,Ca,YZab),(Nbn,Ca,YZac),(Nbn,Ca,YZad),(Nbn,Ca,YZae),(Nbn, Ca,YZaf),(Nbn,Ca,YZag),(Nbn,Cb,YZa),(Nbn,Cb,YZb),(Nbn,Cb,YZc),(Nbn,Cb,YZd),(Nbn, Cb,YZe),(Nbn,Cb,YZf),(Nbn,Cb,YZg),(Nbn,Cb,YZh),(Nbn,Cb,YZi),(Nbn,Cb,YZj),(Nbn,Cb, YZk),(Nbn,Cb,YZl),(Nbn,Cb,YZm),(Nbn,Cb,YZn),(Nbn,Cb,YZo),(Nbn,Cb,YZp),(Nbn,Cb,Y Zq),(Nbn,Cb,YZr),(Nbn,Cb,YZs),(Nbn,Cb,YZt),(Nbn,Cb,YZu),(Nbn,Cb,YZv),(Nbn,Cb,YZ w),(Nbn,Cb,YZx),(Nbn,Cb,YZy),(Nbn,Cb,YZz),(Nbn,Cb,YZaa),(Nbn,Cb,YZab),(Nbn,Cb,Y Zac),(Nbn,Cb,YZad),(Nbn,Cb,YZae),(Nbn,Cb,YZaf),(Nbn,Cb,YZag),(Nbn,Cc,YZa),(Nbn,C c,YZb),(Nbn,Cc,YZc),(Nbn,Cc,YZd),(Nbn,Cc,YZe),(Nbn,Cc,YZf),(Nbn,Cc,YZg),(Nbn,Cc, YZh),(Nbn,Cc,YZi),(Nbn,Cc,YZj),(Nbn,Cc,YZk),(Nbn,Cc,YZl),(Nbn,Cc,YZm),(Nbn,Cc,YZ n),(Nbn,Cc,YZo),(Nbn,Cc,YZp),(Nbn,Cc,YZq),(Nbn,Cc,YZr),(Nbn,Cc,YZs),(Nbn,Cc,YZt),( Nbn,Cc,YZu),(Nbn,Cc,YZv),(Nbn,Cc,YZw),(Nbn,Cc,YZx),(Nbn,Cc,YZy),(Nbn,Cc,YZz),(N bn,Cc,YZaa),(Nbn,Cc,YZab),(Nbn,Cc,YZac),(Nbn,Cc,YZad),(Nbn,Cc,YZae),(Nbn,Cc,YZaf) ,(Nbn,Cc,YZag),(Nbn,Cd,YZa),(Nbn,Cd,YZb),(Nbn,Cd,YZc),(Nbn,Cd,YZd),(Nbn,Cd,YZe), (Nbn,Cd,YZf),(Nbn,Cd,YZg),(Nbn,Cd,YZh),(Nbn,Cd,YZi),(Nbn,Cd,YZj),(Nbn,Cd,YZk),(N bn,Cd,YZl),(Nbn,Cd,YZm),(Nbn,Cd,YZn),(Nbn,Cd,YZo),(Nbn,Cd,YZp),(Nbn,Cd,YZq),(Nb n,Cd,YZr),(Nbn,Cd,YZs),(Nbn,Cd,YZt),(Nbn,Cd,YZu),(Nbn,Cd,YZv),(Nbn,Cd,YZw),(Nbn, Cd,YZx),(Nbn,Cd,YZy),(Nbn,Cd,YZz),(Nbn,Cd,YZaa),(Nbn,Cd,YZab),(Nbn,Cd,YZac),(Nb n,Cd,YZad),(Nbn,Cd,YZae),(Nbn,Cd,YZaf),(Nbn,Cd,YZag),(Nbn,Ce,YZa),(Nbn,Ce,YZb),( Nbn,Ce,YZc),(Nbn,Ce,YZd),(Nbn,Ce,YZe),(Nbn,Ce,YZf),(Nbn,Ce,YZg),(Nbn,Ce,YZh),(Nb n,Ce,YZi),(Nbn,Ce,YZj),(Nbn,Ce,YZk),(Nbn,Ce,YZl),(Nbn,Ce,YZm),(Nbn,Ce,YZn),(Nbn,C e,YZo),(Nbn,Ce,YZp),(Nbn,Ce,YZq),(Nbn,Ce,YZr),(Nbn,Ce,YZs),(Nbn,Ce,YZt),(Nbn,Ce,Y Zu),(Nbn,Ce,YZv),(Nbn,Ce,YZw),(Nbn,Ce,YZx),(Nbn,Ce,YZy),(Nbn,Ce,YZz),(Nbn,Ce,YZ aa),(Nbn,Ce,YZab),(Nbn,Ce,YZac),(Nbn,Ce,YZad),(Nbn,Ce,YZae),(Nbn,Ce,YZaf),(Nbn,Ce, YZag),(Nbn,Cf,YZa),(Nbn,Cf,YZb),(Nbn,Cf,YZc),(Nbn,Cf,YZd),(Nbn,Cf,YZe),(Nbn,Cf,YZ f),(Nbn,Cf,YZg),(Nbn,Cf,YZh),(Nbn,Cf,YZi),(Nbn,Cf,YZj),(Nbn,Cf,YZk),(Nbn,Cf,YZl),(N bn,Cf,YZm),(Nbn,Cf,YZn),(Nbn,Cf,YZo),(Nbn,Cf,YZp),(Nbn,Cf,YZq),(Nbn,Cf,YZr),(Nbn, Cf,YZs),(Nbn,Cf,YZt),(Nbn,Cf,YZu),(Nbn,Cf,YZv),(Nbn,Cf,YZw),(Nbn,Cf,YZx),(Nbn,Cf, YZy),(Nbn,Cf,YZz),(Nbn,Cf,YZaa),(Nbn,Cf,YZab),(Nbn,Cf,YZac),(Nbn,Cf,YZad),(Nbn,Cf, YZae),(Nbn,Cf,YZaf),(Nbn,Cf,YZag),(Nbn,Cg,YZa),(Nbn,Cg,YZb),(Nbn,Cg,YZc),(Nbn,Cg, YZd),(Nbn,Cg,YZe),(Nbn,Cg,YZf),(Nbn,Cg,YZg),(Nbn,Cg,YZh),(Nbn,Cg,YZi),(Nbn,Cg,Y Zj),(Nbn,Cg,YZk),(Nbn,Cg,YZl),(Nbn,Cg,YZm),(Nbn,Cg,YZn),(Nbn,Cg,YZo),(Nbn,Cg,YZ p),(Nbn,Cg,YZq),(Nbn,Cg,YZr),(Nbn,Cg,YZs),(Nbn,Cg,YZt),(Nbn,Cg,YZu),(Nbn,Cg,YZv), (Nbn,Cg,YZw),(Nbn,Cg,YZx),(Nbn,Cg,YZy),(Nbn,Cg,YZz),(Nbn,Cg,YZaa),(Nbn,Cg,YZab) ,(Nbn,Cg,YZac),(Nbn,Cg,YZad),(Nbn,Cg,YZae),(Nbn,Cg,YZaf),(Nbn,Cg,YZag),(Nbn,Ch,Y Za),(Nbn,Ch,YZb),(Nbn,Ch,YZc),(Nbn,Ch,YZd),(Nbn,Ch,YZe),(Nbn,Ch,YZf),(Nbn,Ch,YZg) ,(Nbn,Ch,YZh),(Nbn,Ch,YZi),(Nbn,Ch,YZj),(Nbn,Ch,YZk),(Nbn,Ch,YZl),(Nbn,Ch,YZm),( Nbn,Ch,YZn),(Nbn,Ch,YZo),(Nbn,Ch,YZp),(Nbn,Ch,YZq),(Nbn,Ch,YZr),(Nbn,Ch,YZs),(N bn,Ch,YZt),(Nbn,Ch,YZu),(Nbn,Ch,YZv),(Nbn,Ch,YZw),(Nbn,Ch,YZx),(Nbn,Ch,YZy),(Nb n,Ch,YZz),(Nbn,Ch,YZaa),(Nbn,Ch,YZab),(Nbn,Ch,YZac),(Nbn,Ch,YZad),(Nbn,Ch,YZae),( Nbn,Ch,YZaf),(Nbn,Ch,YZag),(Nbn,Ci,YZa),(Nbn,Ci,YZb),(Nbn,Ci,YZc),(Nbn,Ci,YZd),(N bn,Ci,YZe),(Nbn,Ci,YZf),(Nbn,Ci,YZg),(Nbn,Ci,YZh),(Nbn,Ci,YZi),(Nbn,Ci,YZj),(Nbn,Ci, YZk),(Nbn,Ci,YZl),(Nbn,Ci,YZm),(Nbn,Ci,YZn),(Nbn,Ci,YZo),(Nbn,Ci,YZp),(Nbn,Ci,YZq) ,(Nbn,Ci,YZr),(Nbn,Ci,YZs),(Nbn,Ci,YZt),(Nbn,Ci,YZu),(Nbn,Ci,YZv),(Nbn,Ci,YZw),(Nb n,Ci,YZx),(Nbn,Ci,YZy),(Nbn,Ci,YZz),(Nbn,Ci,YZaa),(Nbn,Ci,YZab),(Nbn,Ci,YZac),(Nbn, Ci,YZad),(Nbn,Ci,YZae),(Nbn,Ci,YZaf),(Nbn,Ci,YZag),(Nbn,Cj,YZa),(Nbn,Cj,YZb),(Nbn, Cj,YZc),(Nbn,Cj,YZd),(Nbn,Cj,YZe),(Nbn,Cj,YZf),(Nbn,Cj,YZg),(Nbn,Cj,YZh),(Nbn,Cj,Y Zi),(Nbn,Cj,YZj),(Nbn,Cj,YZk),(Nbn,Cj,YZl),(Nbn,Cj,YZm),(Nbn,Cj,YZn),(Nbn,Cj,YZo),( Nbn,Cj,YZp),(Nbn,Cj,YZq),(Nbn,Cj,YZr),(Nbn,Cj,YZs),(Nbn,Cj,YZt),(Nbn,Cj,YZu),(Nbn, Cj,YZv),(Nbn,Cj,YZw),(Nbn,Cj,YZx),(Nbn,Cj,YZy),(Nbn,Cj,YZz),(Nbn,Cj,YZaa),(Nbn,Cj, YZab),(Nbn,Cj,YZac),(Nbn,Cj,YZad),(Nbn,Cj,YZae),(Nbn,Cj,YZaf),(Nbn,Cj,YZag),(Nbo,C a,YZa),(Nbo,Ca,YZb),(Nbo,Ca,YZc),(Nbo,Ca,YZd),(Nbo,Ca,YZe),(Nbo,Ca,YZf),(Nbo,Ca,Y Zg),(Nbo,Ca,YZh),(Nbo,Ca,YZi),(Nbo,Ca,YZj),(Nbo,Ca,YZk),(Nbo,Ca,YZl),(Nbo,Ca,YZm) ,(Nbo,Ca,YZn),(Nbo,Ca,YZo),(Nbo,Ca,YZp),(Nbo,Ca,YZq),(Nbo,Ca,YZr),(Nbo,Ca,YZs),(N bo,Ca,YZt),(Nbo,Ca,YZu),(Nbo,Ca,YZv),(Nbo,Ca,YZw),(Nbo,Ca,YZx),(Nbo,Ca,YZy),(Nbo ,Ca,YZz),(Nbo,Ca,YZaa),(Nbo,Ca,YZab),(Nbo,Ca,YZac),(Nbo,Ca,YZad),(Nbo,Ca,YZae),(N bo,Ca,YZaf),(Nbo,Ca,YZag),(Nbo,Cb,YZa),(Nbo,Cb,YZb),(Nbo,Cb,YZc),(Nbo,Cb,YZd),(N bo,Cb,YZe),(Nbo,Cb,YZf),(Nbo,Cb,YZg),(Nbo,Cb,YZh),(Nbo,Cb,YZi),(Nbo,Cb,YZj),(Nbo, Cb,YZk),(Nbo,Cb,YZl),(Nbo,Cb,YZm),(Nbo,Cb,YZn),(Nbo,Cb,YZo),(Nbo,Cb,YZp),(Nbo,C b,YZq),(Nbo,Cb,YZr),(Nbo,Cb,YZs),(Nbo,Cb,YZt),(Nbo,Cb,YZu),(Nbo,Cb,YZv),(Nbo,Cb, YZw),(Nbo,Cb,YZx),(Nbo,Cb,YZy),(Nbo,Cb,YZz),(Nbo,Cb,YZaa),(Nbo,Cb,YZab),(Nbo,Cb ,YZac),(Nbo,Cb,YZad),(Nbo,Cb,YZae),(Nbo,Cb,YZaf),(Nbo,Cb,YZag),(Nbo,Cc,YZa),(Nbo, Cc,YZb),(Nbo,Cc,YZc),(Nbo,Cc,YZd),(Nbo,Cc,YZe),(Nbo,Cc,YZf),(Nbo,Cc,YZg),(Nbo,Cc, YZh),(Nbo,Cc,YZi),(Nbo,Cc,YZj),(Nbo,Cc,YZk),(Nbo,Cc,YZl),(Nbo,Cc,YZm),(Nbo,Cc,YZ n),(Nbo,Cc,YZo),(Nbo,Cc,YZp),(Nbo,Cc,YZq),(Nbo,Cc,YZr),(Nbo,Cc,YZs),(Nbo,Cc,YZt),( Nbo,Cc,YZu),(Nbo,Cc,YZv),(Nbo,Cc,YZw),(Nbo,Cc,YZx),(Nbo,Cc,YZy),(Nbo,Cc,YZz),(N bo,Cc,YZaa),(Nbo,Cc,YZab),(Nbo,Cc,YZac),(Nbo,Cc,YZad),(Nbo,Cc,YZae),(Nbo,Cc,YZaf) ,(Nbo,Cc,YZag),(Nbo,Cd,YZa),(Nbo,Cd,YZb),(Nbo,Cd,YZc),(Nbo,Cd,YZd),(Nbo,Cd,YZe), (Nbo,Cd,YZf),(Nbo,Cd,YZg),(Nbo,Cd,YZh),(Nbo,Cd,YZi),(Nbo,Cd,YZj),(Nbo,Cd,YZk),(N bo,Cd,YZl),(Nbo,Cd,YZm),(Nbo,Cd,YZn),(Nbo,Cd,YZo),(Nbo,Cd,YZp),(Nbo,Cd,YZq),(Nb o,Cd,YZr),(Nbo,Cd,YZs),(Nbo,Cd,YZt),(Nbo,Cd,YZu),(Nbo,Cd,YZv),(Nbo,Cd,YZw),(Nbo, Cd,YZx),(Nbo,Cd,YZy),(Nbo,Cd,YZz),(Nbo,Cd,YZaa),(Nbo,Cd,YZab),(Nbo,Cd,YZac),(Nb o,Cd,YZad),(Nbo,Cd,YZae),(Nbo,Cd,YZaf),(Nbo,Cd,YZag),(Nbo,Ce,YZa),(Nbo,Ce,YZb),( Nbo,Ce,YZc),(Nbo,Ce,YZd),(Nbo,Ce,YZe),(Nbo,Ce,YZf),(Nbo,Ce,YZg),(Nbo,Ce,YZh),(Nb o,Ce,YZi),(Nbo,Ce,YZj),(Nbo,Ce,YZk),(Nbo,Ce,YZl),(Nbo,Ce,YZm),(Nbo,Ce,YZn),(Nbo,C e,YZo),(Nbo,Ce,YZp),(Nbo,Ce,YZq),(Nbo,Ce,YZr),(Nbo,Ce,YZs),(Nbo,Ce,YZt),(Nbo,Ce,Y Zu),(Nbo,Ce,YZv),(Nbo,Ce,YZw),(Nbo,Ce,YZx),(Nbo,Ce,YZy),(Nbo,Ce,YZz),(Nbo,Ce,YZ aa),(Nbo,Ce,YZab),(Nbo,Ce,YZac),(Nbo,Ce,YZad),(Nbo,Ce,YZae),(Nbo,Ce,YZaf),(Nbo,Ce, YZag),(Nbo,Cf,YZa),(Nbo,Cf,YZb),(Nbo,Cf,YZc),(Nbo,Cf,YZd),(Nbo,Cf,YZe),(Nbo,Cf,YZ f),(Nbo,Cf,YZg),(Nbo,Cf,YZh),(Nbo,Cf,YZi),(Nbo,Cf,YZj),(Nbo,Cf,YZk),(Nbo,Cf,YZl),(N bo,Cf,YZm),(Nbo,Cf,YZn),(Nbo,Cf,YZo),(Nbo,Cf,YZp),(Nbo,Cf,YZq),(Nbo,Cf,YZr),(Nbo, Cf,YZs),(Nbo,Cf,YZt),(Nbo,Cf,YZu),(Nbo,Cf,YZv),(Nbo,Cf,YZw),(Nbo,Cf,YZx),(Nbo,Cf, YZy),(Nbo,Cf,YZz),(Nbo,Cf,YZaa),(Nbo,Cf,YZab),(Nbo,Cf,YZac),(Nbo,Cf,YZad),(Nbo,Cf, YZae),(Nbo,Cf,YZaf),(Nbo,Cf,YZag),(Nbo,Cg,YZa),(Nbo,Cg,YZb),(Nbo,Cg,YZc),(Nbo,Cg, YZd),(Nbo,Cg,YZe),(Nbo,Cg,YZf),(Nbo,Cg,YZg),(Nbo,Cg,YZh),(Nbo,Cg,YZi),(Nbo,Cg,Y Zj),(Nbo,Cg,YZk),(Nbo,Cg,YZl),(Nbo,Cg,YZm),(Nbo,Cg,YZn),(Nbo,Cg,YZo),(Nbo,Cg,YZ p),(Nbo,Cg,YZq),(Nbo,Cg,YZr),(Nbo,Cg,YZs),(Nbo,Cg,YZt),(Nbo,Cg,YZu),(Nbo,Cg,YZv), (Nbo,Cg,YZw),(Nbo,Cg,YZx),(Nbo,Cg,YZy),(Nbo,Cg,YZz),(Nbo,Cg,YZaa),(Nbo,Cg,YZab) ,(Nbo,Cg,YZac),(Nbo,Cg,YZad),(Nbo,Cg,YZae),(Nbo,Cg,YZaf),(Nbo,Cg,YZag),(Nbo,Ch,Y Za),(Nbo,Ch,YZb),(Nbo,Ch,YZc),(Nbo,Ch,YZd),(Nbo,Ch,YZe),(Nbo,Ch,YZf),(Nbo,Ch,YZg) ,(Nbo,Ch,YZh),(Nbo,Ch,YZi),(Nbo,Ch,YZj),(Nbo,Ch,YZk),(Nbo,Ch,YZl),(Nbo,Ch,YZm),( Nbo,Ch,YZn),(Nbo,Ch,YZo),(Nbo,Ch,YZp),(Nbo,Ch,YZq),(Nbo,Ch,YZr),(Nbo,Ch,YZs),(N bo,Ch,YZt),(Nbo,Ch,YZu),(Nbo,Ch,YZv),(Nbo,Ch,YZw),(Nbo,Ch,YZx),(Nbo,Ch,YZy),(Nb o,Ch,YZz),(Nbo,Ch,YZaa),(Nbo,Ch,YZab),(Nbo,Ch,YZac),(Nbo,Ch,YZad),(Nbo,Ch,YZae),( Nbo,Ch,YZaf),(Nbo,Ch,YZag),(Nbo,Ci,YZa),(Nbo,Ci,YZb),(Nbo,Ci,YZc),(Nbo,Ci,YZd),(N bo,Ci,YZe),(Nbo,Ci,YZf),(Nbo,Ci,YZg),(Nbo,Ci,YZh),(Nbo,Ci,YZi),(Nbo,Ci,YZj),(Nbo,Ci, YZk),(Nbo,Ci,YZl),(Nbo,Ci,YZm),(Nbo,Ci,YZn),(Nbo,Ci,YZo),(Nbo,Ci,YZp),(Nbo,Ci,YZq) ,(Nbo,Ci,YZr),(Nbo,Ci,YZs),(Nbo,Ci,YZt),(Nbo,Ci,YZu),(Nbo,Ci,YZv),(Nbo,Ci,YZw),(Nb o,Ci,YZx),(Nbo,Ci,YZy),(Nbo,Ci,YZz),(Nbo,Ci,YZaa),(Nbo,Ci,YZab),(Nbo,Ci,YZac),(Nbo, Ci,YZad),(Nbo,Ci,YZae),(Nbo,Ci,YZaf),(Nbo,Ci,YZag),(Nbo,Cj,YZa),(Nbo,Cj,YZb),(Nbo, Cj,YZc),(Nbo,Cj,YZd),(Nbo,Cj,YZe),(Nbo,Cj,YZf),(Nbo,Cj,YZg),(Nbo,Cj,YZh),(Nbo,Cj,Y Zi),(Nbo,Cj,YZj),(Nbo,Cj,YZk),(Nbo,Cj,YZl),(Nbo,Cj,YZm),(Nbo,Cj,YZn),(Nbo,Cj,YZo),( Nbo,Cj,YZp),(Nbo,Cj,YZq),(Nbo,Cj,YZr),(Nbo,Cj,YZs),(Nbo,Cj,YZt),(Nbo,Cj,YZu),(Nbo, Cj,YZv),(Nbo,Cj,YZw),(Nbo,Cj,YZx),(Nbo,Cj,YZy),(Nbo,Cj,YZz),(Nbo,Cj,YZaa),(Nbo,Cj, YZab),(Nbo,Cj,YZac),(Nbo,Cj,YZad),(Nbo,Cj,YZae),(Nbo,Cj,YZaf) and (Nbo,Cj,YZag).

Examplary preferred compounds useful in the present invention include:
N,N-Diethyl-2-[1-(4-trifluoromethoxybenzenesulfonyl)piperidin-4-ylideneaminooxy]acetamide
N-Phenyl-2-[1-(4-trifluoromethoxybenzenesulfonyl)piperidin-4-ylideneaminooxy]acetamide
N-Methyl-N-phenyl-2-[1-(4-trifluoromethoxybenzenesulfonyl)piperidin-4-ylideneaminooxy]acetamide
N-(4-Fluorophenyl)-2-[1-(4-trifluoromethoxybenzenesulfonyppiperidin-4-ylideneaminooxy]acetamide
N-(Thiazol-2-yl)-2-(1-(3-(trifluoromethyl)phenylsulfonyl)piperidin-4-ylideneaminooxy)acetamide
N-Isopropyl-2-(1-(3-(trifluoromethyl)phenylsulfonyl)piperidin-4-ylideneaminooxy)acetamide
N-(6-Methoxypyridin-3-yl)-2-(1-(3-(trifluoromethyl)phenylsulfonyl)piperidin-4-ylideneaminooxy)acetamide
N-(4-Fluorophenyl)-2-(1-(3-(trifluoromethyl)phenylsulfonyl)piperidin-4-ylideneaminooxy)acetamide
N-Cyclopropyl-2-(1-(3-(trifluoromethyl)phenylsulfonyl)piperidin-4-ylideneaminooxy)acetamide
N-(2-Cyanoethyl)-2-(1-(3-(trifluoromethyl)phenylsulfonyl)piperidin-4-ylideneaminooxy)acetamide
N-(3-Chlorophenyl)-2-(1-(3-(trifluoromethyl)phenylsulfonyl)piperidin-4-ylideneaminooxy)acetamide
N-(Pyridin-4-yl)-2-(1-(3-(trifluoromethyl)phenylsulfonyl)piperidin-4-ylideneaminooxy)acetamide
N-(Pyridin-2-yl)-2-(1-(3-(trifluoromethyl)phenylsulfonyl)piperidin-4-ylideneaminooxy)acetamide
N-Benzyl-N-methyl-2-(1-(3-(trifluoromethyl)phenylsulfonyl)piperidin-4-ylideneaminooxy)acetamide
N-(2-Methoxyphenyl)-2-(1-(3-(trifluoromethyl)phenylsulfonyl)piperidin-4-ylideneaminooxy)acetamide
N-(2,2,2-Trifluoroethyl)-2-(1-(3-(trifluoromethyl)phenylsulfonyl)piperidin-4-ylideneaminooxy)acetamide
N-(2-Hydroxyethyl)-N-phenyl-2-(1-(3-(trifluoromethyl)phenylsulfonyl)piperidin-4-ylideneaminooxy)acetamide
N-(4-Acetylphenyl)-2-(1-(3-(trifluoromethyl)phenylsulfonyl)piperidin-4-ylideneaminooxy)acetamide
N-Cyano-2-(1-(3-(trifluoromethyl)phenylsulfonyl)piperidin-4-ylideneaminooxy)acetamide
N-(Pyridin-3-yl)-2-(1-(3-(trifluoromethyl)phenylsulfonyl)piperidin-4-ylideneaminooxy)acetamide
N-tert-Butyl-2-(1-(3-(trifluoromethyl)phenylsulfonyl)piperidin-4-ylideneaminooxy)acetamide
N-(1-Phenylethyl)-2-(1-(3-(trifluoromethyl)phenylsulfonyl)piperidin-4-ylideneaminooxy)acetamide
N-(4-Fluorobenzyl)-2-(1-(3-(trifluoromethyl)phenylsulfonyl)piperidin-4-ylideneaminooxy)acetamide
1-(3,4-Dihydroquinolin-1(2H)-yl)-2-(1-(3-(trifluoromethyl)phenylsulfonyl)piperidin-4-ylideneaminooxy)ethanone
N-Cyclohexyl-2-(1-(3-(trifluoromethyl)phenylsulfonyl)piperidin-4-ylideneaminooxy)acetamide
N-Phenyl-2-(1-(3-(trifluoromethyl)phenylsulfonyl)piperidin-4-ylideneaminooxy)acetamide
N-(2-Phenoxyethyl)-2-(1-(3-(trifluoromethyl)phenylsulfonyl)piperidin-4-ylideneaminooxy)acetamide
N-(2-Methoxy-6-methylphenyl)-2-(1-(3-(trifluoromethyl)phenylsulfonyl)piperidin-4-ylideneaminooxy)acetamide
N-(Pyridin-2-ylmethyl)-2-(1-(3-(trifluoromethyl)phenylsulfonyl)piperidin-4-ylideneaminooxy)acetamide
1-(5,6-Dihydropyridin-1(2H)-yl)-2-(1-(3-(trifluoromethyl)phenylsulfonyl)piperidin-4-ylideneaminooxy)ethanone
N-(4-Methoxyphenyl)-2-(1-(3-(trifluoromethyl)phenylsulfonyl)piperidin-4-ylideneaminooxy)acetamide
2-(1-(3-(Trifluoromethyl)phenylsulfonyl)piperidin-4-ylideneaminooxy)-N-(2,4,4-trimethylpentan-2-yl)acetamide
N-(1,3,4-Thiadiazol-2-yl)-2-(1-(3-(trifluoromethyl)phenylsulfonyl)piperidin-4-ylideneaminooxy)acetamide
N-(2-Cyanoethyl)-N-methyl-2-(1-(3-(trifluoromethyl)phenylsulfonyl)piperidin-4-ylideneaminooxy)acetamide
N-(2,3-Dihydro-1H-inden-1-yl)-2-(1-(3-(trifluoromethyl)phenylsulfonyl)piperidin-4-ylideneaminooxy)acetamide
N-(3-Methoxybenzyl)-2-(1-(3-(trifluoromethyl)phenylsulfonyl)piperidin-4-ylideneaminooxy)acetamide
N-(3-Methylbenzyl)-2-(1-(3-(trifluoromethyl)phenylsulfonyl)piperidin-4-ylideneaminooxy)acetamide
1-(Indolin-1-yl)-2-(1-(3-(trifluoromethyl)phenylsulfonyl)piperidin-4-ylideneaminooxy)ethanone
N-(2,3-Dihydro-1H-inden-2-yl)-2-(1-(3-(trifluoromethyl)phenylsulfonyl)piperidin-4-ylideneaminooxy)acetamide
N-(Pyridin-4-ylmethyl)-2-(1-(3-(trifluoromethyl)phenylsulfonyl)piperidin-4-ylideneaminooxy)acetamide
N-(Pyridin-3-ylmethyl)-2-(1-(3-(trifluoromethyl)phenylsulfonyl)piperidin-4-ylideneaminooxy)acetamide
N-(2-Morpholinoethyl)-2-(1-(3-(trifluoromethyl)phenylsulfonyl)piperidin-4-ylideneaminooxy)acetamide
N-Butyl-2-(1-(3-(trifluoromethyl)phenylsulfonyl)piperidin-4-ylideneaminooxy)acetamide
N-Cyclohexyl-2-(2-(1-(3-(trifluoromethyl)phenylsulfonyl)piperidin-4-ylideneaminooxy)acetamido)benzamide
1-(2-Methylpiperidin-1-yl)-2-(1-(3-(trifluoromethyl)phenylsulfonyl)piperidin-4-ylideneaminooxy)ethanone
1-(3-Hydroxypiperidin-1-yl)-2-(1-(3-(trifluoromethyl)phenylsulfonyl)piperidin-4-ylideneaminooxy)ethanone
1-(4-Hydroxypiperidin-1-yl)-2-(1-(3-(trifluoromethyl)phenylsulfonyl)piperidin-4-ylideneaminooxy)ethanone
1-(3,5-Dimethylpiperidin-1-yl)-2-(1-(3-(trifluoromethyl)phenylsulfonyl)piperidin-4-ylideneaminooxy)ethanone
1-(4-(Hydroxymethyl)piperidin-1-yl)-2-(1-(3-(trifluoromethyl)phenylsulfonyl)piperidin-4-ylideneaminooxy)ethanone
1-(3-Hydroxypyrrolidin-1-yl)-2-(1-(3-(trifluoromethyl)phenylsulfonyl)piperidin-4-ylideneaminooxy)ethanone
N-(1H-Indol-5-yl)-2-(1-(3-(trifluoromethyl)phenylsulfonyl)piperidin-4-ylideneaminooxy)acetamide
N-Benzyl-2-(1-(3-(trifluoromethyl)phenylsulfonyl)piperidin-4-ylideneaminooxy)acetamide
N-(4-Cyanophenyl)-2-(1-(3-(trifluoromethyl)phenylsulfonyl)piperidin-4-ylideneaminooxy)acetamide
N-(3-Fluorobenzyl)-2-(1-(3-(trifluoromethyl)phenylsulfonyl)piperidin-4-ylideneaminooxy)acetamide
1-Morpholino-2-(1-(3-(trifluoromethyl)phenylsulfonyl)piperidin-4-ylideneaminooxy)ethanone
N-(4-Chlorophenyl)-2-(1-(3-(trifluoromethyl)phenylsulfonyl)piperidin-4-ylideneaminooxy)acetamide
N-(1-Hydroxypropan-2-yl)-2-(1-(3-(trifluoromethyl)phenylsulfonyl)piperidin-4-ylideneaminooxy)acetamide
N-(2-Fluoroethyl)-2-(1-(3-(trifluoromethyl)phenylsulfonyl)piperidin-4-ylideneaminooxy)acetamide
N-(3,3-Dimethylbutyl)-2-(1-(3-(trifluoromethyl)phenylsulfonyl)piperidin-4-ylideneaminooxy)acetamide
N-Methyl-N-propyl-2-(1-(3-(trifluoromethyl)phenylsulfonyl)piperidin-4-ylideneaminooxy)acetamide
N-(2-Hydroxyethyl)-N-methyl-2-(1-(3-(trifluoromethyl)phenylsulfonyl)piperidin-4-ylideneaminooxy)acetamide
N-(1-Hydroxy-2-methylpropan-2-yl)-2-(1-(3-(trifluoromethyl)phenylsulfonyl)piperidin-4-ylideneaminooxy)acetamide
N-Propyl-2-(1-(3-(trifluoromethyl)phenylsulfonyl)piperidin-4-ylideneaminooxy)acetamide
N,N-Diisopropyl-2-(1-(3-(trifluoromethyl)phenylsulfonyl)piperidin-4-ylideneaminooxy)acetamide
N-sec-Butyl-2-(1-(3-(trifluoromethyl)phenylsulfonyl)piperidin-4-ylideneaminooxy)acetamide
N-(Pentan-3-yl)-2-(1-(3-(trifluoromethyl)phenylsulfonyl)piperidin-4-ylideneaminooxy)acetamide
N-Neopentyl-2-(1-(3-(trifluoromethyl)phenylsulfonyl)piperidin-4-ylideneaminooxy)acetamide
N-Isopentyl-2-(1-(3-(trifluoromethyl)phenylsulfonyl)piperidin-4-ylideneaminooxy)acetamide
N-Isobutyl-N-methyl-2-(1-(3-(trifluoromethyl)phenylsulfonyl)piperidin-4-ylideneaminooxy)acetamide
N-(2-Hydroxybutyl)-2-(1-(3-(trifluoromethyl)phenylsulfonyl)piperidin-4-ylideneaminooxy)acetamide
N-(3-Hydroxy-2,2-dimethylpropyl)-2-(1-(3-(trifluoromethyl)phenylsulfonyl)piperidin-4-ylideneaminooxy)acetamide
N-(2-Methoxyethyl)-2-(1-(3-(trifluoromethyl)phenylsulfonyl)piperidin-4-ylideneaminooxy)acetamide
N-(3,3-Dimethylbutan-2-yl)-2-(1-(3-(trifluoromethyl)phenylsulfonyl)piperidin-4-ylideneaminooxy)acetamide
N-(2-Hydroxypropyl)-2-(1-(3-(trifluoromethyl)phenylsulfonyl)piperidin-4-ylideneaminooxy)acetamide
N-Isobutyl-2-(1-(3-(trifluoromethyl)phenylsulfonyl)piperidin-4-ylideneaminooxy)acetamide
N-(3-Hydroxypropyl)-2-(1-(3-(trifluoromethyl)phenylsulfonyl)piperidin-4-ylideneaminooxy)acetamide
N-(Cyanomethyl)-2-(1-(3-(trifluoromethyl)phenylsulfonyl)piperidin-4-ylideneaminooxy)acetamide
N-(2-Fluorophenyl)-2-(1-(3-(trifluoromethyl)phenylsulfonyl)piperidin-4-ylideneaminooxy)acetamide
N-(4-Hydroxycyclohexyl)-2-(1-(3-(trifluoromethyl)phenylsulfonyl)piperidin-4-ylideneaminooxy)acetamide
N′,N′-Dimethyl-2-(1-(3-(trifluoromethyl)phenylsulfonyl)piperidin-4-ylideneaminooxy)acetohydrazide
N-(Pyrimidin-4-yl)-2-(1-(3-(trifluoromethyl)phenylsulfonyl)piperidin-4-ylideneaminooxy)acetamide
N-Cyclobutyl-2-(1-(3-(trifluoromethyl)phenylsulfonyl)piperidin-4-ylideneaminooxy)acetamide
N-(3-Cyanophenyl)-2-(1-(3-(trifluoromethyl)phenylsulfonyl)piperidin-4-ylideneaminooxy)acetamideN-(3-Fluorophenyl)-2-(1-(3-(trifluoromethyl)phenylsulfonyl)piperidin-4-ylideneaminooxy)acetamide
N-(3-Methoxyphenyl)-2-(1-(3-(trifluoromethyl)phenylsulfonyl)piperidin-4-ylideneaminooxy)acetamide
N-((Tetrahydro-2H-pyran-4-yl)methyl)-2-(1-(3-(trifluoromethyl)phenylsulfonyl)piperidin-4-ylideneaminooxy)acetamide
N-(5-Methylhexan-2-yl)-2-(1-(3-(trifluoromethyl)phenylsulfonyl)piperidin-4-ylideneaminooxy)acetamide
N-(2,3-Dihydrobenzofuran-5-yl)-2-(1-(3-(trifluoromethyl)phenylsulfonyl)piperidin-4-ylideneaminooxy)acetamide
N-(Thiazol-2-ylmethyl)-2-(1-(3-(trifluoromethyl)phenylsulfonyl)piperidin-4-ylideneaminooxy)acetamide
1-(Isoindolin-2-yl)-2-(1-(3-(trifluoromethyl)phenylsulfonyl)piperidin-4-ylideneaminooxy)ethanone
N-(1-Methoxypropan-2-yl)-2-(1-(3-(trifluoromethyl)phenylsulfonyl)piperidin-4-ylideneaminooxy)acetamide
N-Ethyl-2-(1-(3-(trifluoromethyl)phenylsulfonyl)piperidin-4-ylideneaminooxy)acetamide
N-(6-Fluoropyridin-3-yl)-2-(1-(3-(trifluoromethyl)phenylsulfonyl)piperidin-4-ylideneaminooxy)acetamide
1-(4-Trifluoromethoxybenzenesulfonyl)piperidin-4-one O-(2-oxo-2-pyrrolidin-1-ylethyl)oxime
N-Methyl-2-[1-(4-trifluoromethoxybenzenesulfonyl)piperidin-4-ylideneaminooxy]acetamide
N,N-Dimethyl-2-[1-(4-trifluoromethoxybenzenesulfonyl)piperidin-4-ylideneaminooxy]acetamide
1-(4-Trifluoromethoxybenzenesulfonyl)piperidin-4-one O-(2-oxo-2-piperidin-1-ylethyl)oxime
N-Benzyl-2-[1-(4-trifluoromethoxybenzenesulfonyl)piperidin-4-ylideneaminooxy]acetamide
N-Isobutyl-2-[1-(4-trifluoromethoxybenzenesulfonyl)piperidin-4-ylideneaminooxy]acetamide
N-Cyclohexyl-2-[1-(4-trifluoromethoxybenzenesulfonyl)piperidin-4-ylideneaminooxy]acetamide
N-(2-Hydroxyethyl)-2-[1-(4-trifluoromethoxybenzenesulfonyl)piperidin-4-ylideneaminooxy]acetamide
N-Propyl-2-[1-(4-trifluoromethoxybenzenesulfonyl)piperidin-4-ylideneaminooxy]acetamide
3-(2-morpholino-2-oxo-1-(1-(4-(trifluoromethoxy)phenylsulfonyl)piperidin-4-ylideneaminooxy)ethyl)benzonitrile

The invention disclosed herein is also meant to encompass prodrugs of the disclosed compounds. Prodrugs are considered to be any covalently bonded carriers that release the active parent drug in vivo. Non-limiting examples of prodrugs include esters or amides of compounds of Formula I, I′, or I″ having hydroxy or amino as a substituent, and these can be prepared by reacting such compounds with anhydrides such as succinic anhydride.

The invention disclosed herein is also meant to encompass the disclosed compounds being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H, ³H, ¹¹C, ¹³C, ¹⁴C, ¹⁵N, ¹⁸O, ¹⁷O, ³¹P, ³²P, ³⁵S, ¹⁸F, and ³⁶Cl, respectively, and preferably ³H, ¹¹C, and ¹⁴C. Isotopically-labeled compounds of the present invention can be prepared by methods known in the art.

The present invention is also directed specifically to ³H, ¹¹C, and ¹⁴C radiolabeled compounds of Formula I, I′, or I″, as well as their pharmaceutically acceptable salts, prodrugs and solvates, and the use of any such compounds as radioligands for their binding site on the calcium channel. For example, one use of the labeled compounds of the present invention is the characterization of specific receptor binding. Another use of the labeled compounds of the present invention is an alternative to animal testing for the evaluation of structure-activity relationships. For example, the receptor assay may be performed at a fixed concentration of a labeled compound of Formula I, I′, or I″ and at increasing concentrations of a test compound in a competition assay. For example, tritiated compounds of any of Formula I, I′, or I″ can be prepared by introducing tritium into the particular compound of Formula I, I′, or I″, for example, by catalytic dehalogenation with tritium. This method may include reacting a suitably halogen-substituted precursor of a compound of Formula I, I′, or I″ with tritium gas in the presence of a suitable catalyst, for example, Pd/C, in the presence or absence of a base. Other suitable methods for preparing tritiated compounds can be found in Filer, Isotopes in the Physical and Biomedical Sciences, Vol. 1, Labeled Compounds (Part A), Chapter 6 (1987). ¹⁴C-labeled compounds can be prepared by employing starting materials having a ¹⁴C carbon.

Some of the compounds disclosed herein may contain one or more asymmetric centers and may thus give rise to enantiomers, diastereomers, and other stereoisomeric forms. The present invention is meant to encompass the uses of all such possible forms, as well as their racemic and resolved forms and mixtures thereof. The individual enantiomers may be separated according to methods known to those of ordinary skill in the art in view of the present disclosure. When the compounds described herein contain olefinic double bonds or other centers of geometric asymmetry, and unless specified otherwise, it is intended that they include both E and Z geometric isomers. All tautomers are intended to be encompassed by the present invention as well.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive.

The term “resolution” refers to the separation or concentration or depletion of one of the two enantiomeric forms of a molecule.

The terms “a” and “an” refer to one or more.

The invention disclosed herein also encompasses the use of all salts of the disclosed compounds, including all non-toxic pharmaceutically acceptable salts thereof of the disclosed compounds. Examples of pharmaceutically acceptable addition salts include inorganic and organic acid addition salts and basic salts. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, hydrofluoride, phosphate, sulfate, nitrate and the like; organic acid salts such as citrate, lactate, tartrate, maleate, fumarate, mandelate, acetate, dichloroacetate, trifluoroacetate, oxalate, formate, succinate, and the like; sulfonates such as methanesulfonate, benzenesulfonate, p-toluenesulfonate and the like; and amino acid salts such as arginate, asparginate, glutamate and he like.

Acid addition salts can be formed by mixing a solution of the particular compound of the present invention with a solution of a pharmaceutically acceptable non-toxic acid such as hydrochloric acid, fumaric acid, maleic acid, succinic acid, acetic acid, citric acid, tartaric acid, carbonic acid, phosphoric acid, oxalic acid, dichloroacetic acid, and the like. Basic salts can be formed by mixing a solution of the particular compound of the present invention with a solution of a pharmaceutically acceptable non-toxic base such as sodium hydroxide, potassium hydroxide, choline hydroxide, sodium carbonate and the like.

The invention disclosed herein is also meant to encompass solvates of the disclosed compounds. One type of solvate is a hydrate. Solvates typically do not contribute significantly to the physiological activity or toxicity of the compounds and as such can function as pharmacological equivalents.

Some compounds of the present invention may have one or more of the following characteristics:
high affinity for calcium (Ca²⁺) channels, especially N-type calcium channels,
high selectivity to calcium (Ca²⁺) channels, especially N-type calcium channels versus other channels,
reduced side effect,
high stability
high oral absorbability,
high bioavailability,
low clearance,
easily transfers to brain
long half-life,
long efficacy of a medicine and/or
high protein-unbound fraction.

These compounds are considered useful as blockers of calcium(Ca2+) channels, especially N-type calcium channels.

Since compounds of Formula I, I′, or I″ are blockers of calcium (Ca²⁺) channels, a number of diseases and conditions mediated by calcium ion influx can be treated by employing these compounds. Therefore, the present invention provides a method of treating, preventing or ameliorating stroke, neuronal damage resulting from head trauma, epilepsy, pain (e.g., chronic pain, chronic pain, which includes but is not limited to, neuropathic pain and inflammatory pain or surgical pain), migraine, a mood disorder, schizophrenia, a neurodegenerative disorder (e.g., Alzheimer's disease, amyotrophic lateral sclerosis (ALS), or Parkinson's disease), depression, anxiety, a psychosis, hypertension, or cardiac arrhythmia. In one embodiment, the invention provides a method of treating pain. In another embodiment, the type of pain treated is chronic pain. In another embodiment, the type of pain treated is neuropathic pain. In another embodiment, the type of pain treated is inflammatory pain. In another embodiment, the type of pain treated is acute pain. In each instance, such method of treatment, prevention, or amelioration require administering to an animal in need of such treatment, prevention or amelioration an amount of a compound of the present invention that is therapeutically effective in achieving said treatment, prevention or amelioration. In one embodiment, the amount of such compound is the amount that is effective as to block calcium channels in vivo.

Chronic pain includes, but is not limited to, neuropathic pain, inflammatory pain, postoperative pain, cancer pain, osteoarthritis pain associated with metastatic cancer, trigeminal neuralgia, acute herpetic and postherpetic neuralgia, diabetic neuropathy, causalgia, brachial plexus avulsion, occipital neuralgia, reflex sympathetic dystrophy, fibromyalgia, gout, phantom limb pain, burn pain, and other forms of neuralgia, neuropathic, and idiopathic pain syndromes.

Chronic somatic pain generally results from inflammatory responses to tissue injury such as nerve entrapment, surgical procedures, cancer or arthritis (Brower, Nature Biotechnology 2000; 18: 387-391).

The inflammatory process is a complex series of biochemical and cellular events activated in response to tissue injury or the presence of foreign substances (Levine, Inflammatory Pain, In: Textbook of Pain, Wall and Melzack eds., 3^{rd} ed., 1994). Inflammation often occurs at the site of injured tissue, or foreign material, and contributes to the process of tissue repair and healing. The cardinal signs of inflammation include erythema (redness), heat, edema (swelling), pain and loss of function (ibid.). The majority of patients with inflammatory pain do not experience pain continually, but rather experience enhanced pain when the inflamed site is moved or touched. Inflammatory pain includes, but is not limited to, osteoarthritis and rheumatoid arthritis.

Chronic neuropathic pain is a heterogenous disease state with an unclear etiology. In chronic neuropathic pain, the pain can be mediated by multiple mechanisms. This type of pain generally arises from injury to the peripheral or central nervous tissue. The syndromes include pain associated with spinal cord injury, multiple sclerosis, post-herpetic neuralgia, trigeminal neuralgia, phantom pain, causalgia, and reflex sympathetic dystrophy and lower back pain. The chronic pain is different from acute pain in that patients suffer the abnormal pain sensations that can be described as spontaneous pain, continuous superficial burning and/or deep aching pain. The pain can be evoked by heat-, cold-, and mechano-hyperalgesia or by heat-, cold-, or mechano-allodynia.

Neuropathic pain can be caused by injury or infection of peripheral sensory nerves. It includes, but is not limited to, pain from peripheral nerve trauma, herpes virus infection, diabetes mellitus, causalgia, plexus avulsion, neuroma, limb amputation, and vasculitis. Neuropathic pain is also caused by nerve damage from chronic alcoholism, human immunodeficiency virus infection, hypothyroidism, uremia, or vitamin deficiences. Stroke (spinal or brain) and spinal cord injury can also induce neuropathic pain. Cancer-related neuropathic pain results from tumor growth compression of adjacent nerves, brain, or spinal cord. In addition, cancer treatments, including chemotherapy and radiation therapy, can also cause nerve injury. Neuropathic pain includes but is not limited to pain caused by nerve injury such as, for example, the pain from which diabetics suffer.

The present invention is also directed more generally to a method for treating a disorder responsive to the blockade of calcium channels, and particularly the selective blockade of N-type calcium channels, in an animal suffering from said disorder, said method comprising administering to the animal an effective amount of a compound represented by any of defined Formula I, I′, or I″, or a pharmaceutically acceptable salt, prodrug or solvate thereof.

The present invention is also directed to the use of a compound represented by any of defined Formula I, I′, or I″, or a pharmaceutically acceptable salt, prodrug or solvate thereof, in the manufacture of a medicament for treating a disorder responsive to the blockade of calcium channels in an animal suffering from said disorder. In one embodiment, the disorder is responsive to the selective blockade of N-type calcium channels.

Furthermore, the present invention is directed to a method of modulating calcium channels, especially N-type calcium channels, in an animal in need thereof, said method comprising administering to the animal at least one compound represented by any of defined Formula I, I′, or I″, or a pharmaceutically acceptable salt, prodrug or solvate thereof.

The present invention is also directed to the use of a compound represented by any of defined Formula I, I′, or I″, or a pharmaceutically acceptable salt, prodrug or solvate thereof, in the manufacture of a medicament for modulating calcium channels, especially N-type calcium channels, in an animal in need thereof.

### Synthesis of Compounds

The compounds of the present invention can be prepared in a number of ways well known to those skilled in the art of organic synthesis. The compounds of the present invention can be synthesized using the methods outlined below, together with methods known in the art of synthetic organic chemistry, or variations thereof as appreciated by those skilled in the art. Preferred methods include, but are not limited to, those described below. The novel compounds of formula (I) may be prepared using the reactions and techniques described in this section. The reactions are performed in solvents appropriate to the reagents and materials employed and suitable for the transformations being effected. Also, in the synthetic methods described below, it is to be understood that all proposed reaction conditions, including choice of solvent, reaction atmosphere, reaction temperature, duration of experiment and work-up procedures, are chosen to be conditions of standard for that reaction, which should be readily recognized by one skilled in the art. It is understood by one skilled in the art of organic synthesis that the functionality present on various portions of the starting molecule in a reaction must be compatible with the reagents and reactions proposed. Not all compounds of formula (I) falling into a given class may be compatible with some of the reaction conditions required in some of the methods described. Such restrictions to the substituents which are compatible with the reaction conditions will be readily apparent to one skilled in the art and alternative methods can be used. The compounds of formula (I) can be prepared by techniques and procedures readily available to one skilled in the art, for example by following the procedures as set forth in the following Schemes. These Schemes are not intended to limit the scope of the invention in any way. All substituents, unless otherwise indicated, are previously defined. The reagents and starting materials are readily available to one skilled in the art.

Purity of compounds was verified by LCMS measurement. LCMS methods are as follows;

(Method A) Column: Phenomemex Luna C18 (4.6×50 mm, 5 micron particle size), Temperature: 50° C., Pressure limit: 400 bar, Monitored at OD 254 nm, reference 360 nm, Flow rate: 2 ml/min.

HPLC Gradient (Buffer A=0.1% HCO₂H/H₂O, Buffer B=0.1% HCO₂H/CH₃CN)

| Time (min.) | % B |
|---|---|
| 0 | 15 |
| 1.9 | 45 |
| 4.3 | 45 |
| 8.3 | 95 |
| 11.3 | 95 |
| 11.4 | 15 |
| 15.4 | 15 |

(Method B) Column: Discovery HS C18 (4.6×150 mm, 3 micron particle size), Temperature: 25° C., Pressure limit: 400 bar, Monitored at OD 260 nm, reference 360 nm, Flow rate: 1 ml/min.

HPLC Gradient (Buffer A=0.1% TFA/H₂O, Buffer B=0.1% TFA/CH₃CN)

| Time (min.) | % B |
|---|---|
| 0 | 15 |
| 1.9 | 45 |
| 4.3 | 45 |
| 8.3 | 95 |
| 11.3 | 95 |
| 11.4 | 15 |
| 15.4 | 15 |

(Method C) Column: Phenomemex Luna C18 (4.6×50 mm, 5 micron particle size), Temperature: 50° C., Pressure limit: 344.75 bar, Monitored at OD 254 nm, Flow rate: 3 ml/min.

HPLC Gradient (Buffer A=0.1% HCO₂H/H₂O, Buffer B=0.1% HCO₂H/CH₃CN)

| Time (min.) | % B |
|---|---|
| 0 | 10 |
| 3.0 | 100 |
| 4.0 | 100 |

In order to generate compounds of general formula (I) a multi-step reaction sequence as described in Scheme 1 may be employed. Herein, a piperidone or the corresponding equivalent (1a), wherein X and p are as defined above, is reacted with an acid (Z—Y—OH, Y: C═O), wherein Z is as defined above. Typically the reaction is effected using standard amide coupling conditions, familiar to one skilled in the art, such as N-ethyldimethylaminopropylcarbodiimide hydrochloride (WSCD.HCl)/HOBt. The acid chloride (Z—Y—Cl, Y: C═O), acid anhydride (Z—Y—Z, Y: C═O) or sulfonyl chloride (Z—Y—Cl, Y: S═O) may also be coupled to amine (1a) by using standard conditions, familiar to one skilled in the art. The resultant ketone (1b) may then be coupled with hydroxylamine or its salt in a suitable solvent such as ethanol (EtOH), in the presence of a base such as sodium acetate to yield the hydroxime (1c). Alkylation of the hydroxime (1c) with a halide or the corresponding equivalent (R³R⁴N—C(═O)—(CR¹R²)ₙ-hal; hal=Cl, Br, I, OTs etc.), wherein R¹, R², R³, R⁴ and n are as defined above, in a suitable solvent such as CH3CN, in the presence of a base such as potassium carbonate may generate the desired oxime (I).

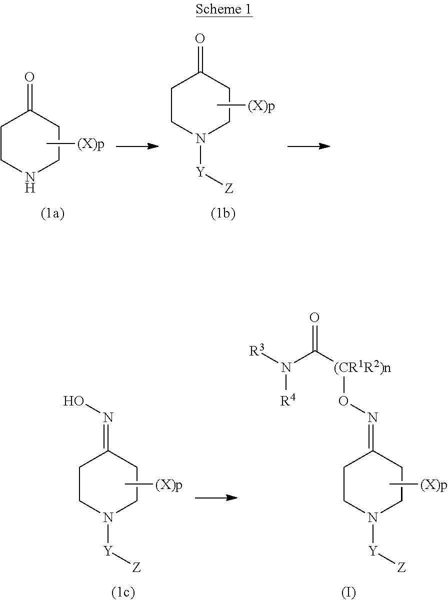

wherein all of the symbols are the same as defined above.

Alternative way of preparing some of the compounds of the present invention is detailed in Scheme 2.

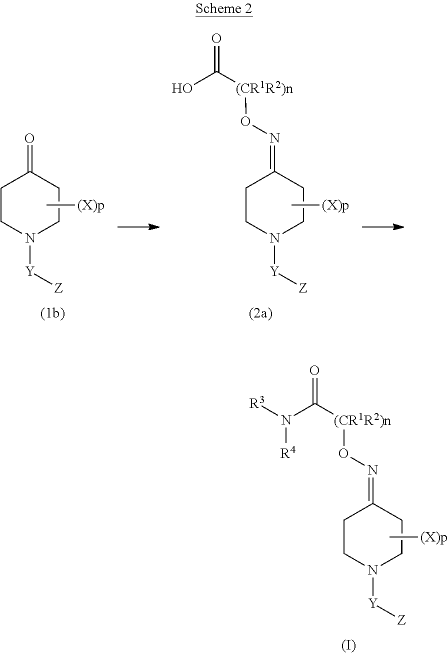

wherein all of the symbols are the same as defined above.

As an alternative to Scheme 1, Scheme 2 employs a carboxylic acid (2a) or the corresponding equivalent which can be coupled with the ketone (1b) and a carboxylated hydroxylamine wherein R¹, R² and n are as defined above, in a suitable solvent such as pyridine. The resulting carboxylic acid (2a) can be coupled with an amine wherein R³ and R⁴ are as defined above, using standard amide coupling conditions, familiar to one skilled in the art, such as O-(benzotriazol-1-yl)-N,N,N′,N′-tetramethyluronium hexafluorophosphate in the presence of triethylamine or N-ethyldimethylaminopropylcarbodiimide hydrochloride (WSCD.HCl)/HOBt to yield the desired oxime (I).

### Testing of Compounds

Representative compounds of the present invention were assessed by calcium mobilization and/or electrophysiological assays for calcium channel blocker activity. One aspect of the present invention is based on the use of the compounds herein described as selective N-type calcium channel blockers. In one aspect of the present invention, it has been found that certain compounds herein described show selectivity as N-type calcium channel blockers. Based upon this property, these compounds are considered useful in treating, preventing, or ameliorating migraine, epilepsy, a mood disorder, schizophrenia, a neurodegenerative disorder (such as, e.g., Alzheimer's disease, ALS, or Parkinson's disease), a psychosis, depression, anxiety, hypertension, or cardiac arrhythmia. The compounds of the present invention are also expected to be effective in treating, preventing or ameliorating pain, such as acute pain, chronic pain, which includes but is not limited to, neuropathic pain and inflammatory pain or surgical pain.

More specifically, the present invention is directed to compounds of Formula I, I′, or I″ that are blockers of calcium channels. According to the present invention, those compounds having preferred N-type calcium channel blocking properties exhibit an IC₅₀ of about 100 μM or less in the calcium mobilization and/or electrophysiological assays described herein. Preferably, the compounds of the present invention exhibit an IC₅₀ of 10 μM or less. Most preferably, the compounds of the present invention exhibit an IC₅₀ of about 1.0 μM or less. Compounds of the present invention can be tested for their N-type and L-type Ca²⁺ channel blocking activity by the following calcium mobilization and/or electrophysiological assays.

In one embodiment, compounds useful in the present invention are those represented by any one of Formula I, I′, or I″ that exhibit selectivity for N-type calcium channels over L-type calcium channels in the calcium mobilization and/or electrophysiological assays described herein. The phrase “selectivity for N-type calcium channels over L-type calcium channels” is used herein to mean that the ratio of an IC₅₀ for L-type channel blocking activity for a compound of the present invention over an IC₅₀ for N-type channel blocking activity for the same compound is more than 1, i.e., LTCC IC₅₀/NTCC IC₅₀>1. Preferably, compounds of the present invention exhibit an LTCC IC₅₀/NTCC IC₅₀ ratio of about 2 or more, about 10 or more, about 20 or more, about 30 or more, about 50 or more, or about 100 or more.

### Calcium Mobilization and Electrophysiological Assay Protocols:

Cell maintenance and differentiation. Unless noted otherwise, cell culture reagents were purchased from Mediatech of Herndon, Md. IMR32 cells (American Type Culture Collection, ATCC, Manassas, Va.) were routinely cultured in growth medium consisting of minimum essential medium containing 10% fetal bovine serum (FBS, Hyclone, Logan, Utah), 100 U/mL penicillin, 100 μg/mL streptomycin, 2 mM L-glutamine, 1 mM sodium pyruvate, and 1× MEM non-essential amino acids. 80-90% confluent flasks of cells were differentiated using the following differentiation medium: Growth medium plus 1 mM dibutyryl cyclic AMP (Sigma, St. Louis, Mo.), and 2.5 μM bromodeoxyuridine (Sigma). Cells were differentiated for 8 days by replacing differentiation medium every 2-3 days.

A7r5 (ATCC) cells were maintained and routinely cultured in A7r5 growth medium consisting of Dulbecco's Modified Eagles Medium containing 10% FBS, 100 U/mL penicillin, 100 μg/mL streptomycin, 4 mM L-glutamine, and 0.15% sodium bicarbonate. 80-90% confluent flasks of cells were differentiated using the following differentiation medium: A7r5 Growth Medium plus 1 mM dibutyryl cyclic AMP (Sigma). Cells were differentiated for 8 days by replacing differentiation medium every 2-3 days.

Recombinant human embryonal kidney cells (HEK293, ATCC) stably transfected with either N-type calcium channel (NTCC) subunits (α1b, α2δ, and β3) or L-type calcium channel (LTCC) subunits (α1c, α2δ, and β1) were routinely cultured in growth medium consisting of Dulbecco's Modified Eagles Medium containing 10% FBS, 100 U/mL penicillin, 100 μg/mL streptomycin, 4 mM L-glutamine, 500 μg/mL geneticin (G418), 20 μg/mL Blasticidin S (InVivogen, San Diego, Calif.) and 500 μg/mL zeocin (InVivogen).

FLIPR Calcium Mobilization Assay for N-type Calcium Channel. One day prior to performing this assay, differentiated IMR32 cells were treated with 1× CellStripper, and seeded on poly-D-lysine-coated 96-well clear-bottom black plates (Becton Dickinson, Franklin Lakes, N.J.) at 200,000 cells/well. On the day of the assay, the cell plates were washed with IMR32 buffer (127 mM NaCl, 1 mM KCl, 2 mM MgCl₂, 700 μM NaH₂PO₄, 5 mM CaCl₂, 5 mM NaHCO₃, 8 mM HEPES, 10 mM glucose, pH 7.4), then pre-stimulated with KCl and loaded as follows: 0.05 mL of IMR32 buffer, 0.05 mL of each compound tested diluted in IMR32 buffer containing 20 μM nitrendipine (Sigma), and 0.1 mL KCl dissolved in IMR32 buffer, plus Fluo-4 were added (3 μM final concentration, Molecular Probes, Eugene, Oreg.). Final test compound concentrations ranged from about 846 pM to about 17 μM, final nitrendipine concentration was 5 μM, and final KCl concentration was 90 mM. After 1 hour, the cells were washed twice with 0.05 mL of each compound tested in nitrendipine-containing IMR32 buffer (no KCl or Fluo-4), and then replaced with 0.1 mL of each compound tested in nitrendipine-containing IMR32 buffer. Plates were then transferred to a Fluorimetric Imaging Plate Reader (FLIPR⁹⁶, Molecular Devices, Inc., Sunnyvale, Calif.) for assay. The FLIPR measured basal Fluo-4 fluorescence for 315 seconds (i.e., 5 minutes and 15 seconds), then added 0.1 mL KCl agonist dissolved in IMR32 buffer and measured fluorescence for another 45 seconds. Final test compound concentrations on the cells after FLIPR read ranged from about 846 pM to about 17 μM, final nitrendipine concentration was 5 μM, and final KCl concentration was 90 mM. Data were collected over the entire time course and analyzed using Excel, Graph Pad Prism (version 3.02, Graph Pad, San Diego, Calif.), or an in-house non-linear regression analysis software.

FLIPR Calcium Mobilization Assay for L-type Calcium Channel. One day prior to performing this assay, HEK293 cells stably expressing recombinant rat L-type calcium channel (LTCC) subunits (α1c, α2δ, and β1) were trypsinized, then seeded on poly-D-lysine-coated 96-well clear-bottom black plates (Becton Dickinson, Franklin Lakes, N.J.) at 75,000 cells/well. On the day of the assay, the plates were washed with LTCC wash buffer (127 mM NaCl, 2 mM MgCl₂, 700 μM NaH₂PO₄, 5 mM CaCl₂, 5 mM NaHCO₃, 8 mM HEPES, 10 mM glucose, pH 7.4), then loaded with 0.1 mL of LTCC wash buffer containing Fluo-4 (3 μM final concentration, Molecular Probes, Eugene, Oreg.). After 1 hour, the cells were washed with 0.1 mL LTCC wash buffer and resuspended in 0.05 mL LTCC assay buffer (same composition as LTCC wash buffer). Plates were then transferred to a FLIPR⁹⁶ for assay. The FLIPR measured basal Fluo-4 fluorescence for 15 seconds, then added 0.05 mL of each compound tested diluted in LTCC assay buffer at final concentrations ranging from about 846 pM to about 17 μM. Fluo-4 fluorescence was then measured for 5 minutes. 0.1 mL KCl agonist dissolved in LTCC assay buffer was then added to the cells to produce a final concentration of 90 mM KCl, and fluorescence was measured for another 45 seconds. Data were collected over the entire time course and analyzed using Excel, Graph Pad Prism, or an in-house regression analysis software.

Alternative FLIPR Calcium Mobilization Assay for L-type Calcium Channel. Alternatively, the following cell line and procedure may be used for the FLIPR calcium mobilization assay for L-type calcium channel. One day prior to performing this assay, differentiated A7r5 cells are trypsinized, then seeded on tissue culture treated 96-well clear-bottom black plates (Becton Dickinson, Franklin Lakes, N.J.) at a dilution of 1:1 from a confluent T150 cm² flask. On the day of the assay, the plates are washed with A7r5 wash buffer (127 mM NaCl, 2 mM MgCl₂, 700 μM NaH₂PO₄, 5 mM CaCl₂, 5 mM NaHCO₃, 8 mM HEPES, 10 mM glucose, pH 7.4), then loaded with 0.1 mL of A7r5 wash buffer containing Fluo-4 (3 μM final concentration, Molecular Probes, Eugene, Oreg.). After 1 hour, the cells are washed with 0.1 mL A7r5 wash buffer and resuspended in 0.05 mL A7r5 assay buffer that is composed of A7r5 wash buffer plus 50 μM valinomycin (Sigma). Plates are then transferred to a FLIPR⁹⁶ for assay. The FLIPR measures basal Fluo-4 fluorescence for 15 seconds, then adds 0.05 mL of each compound tested diluted in A7r5 assay buffer at final concentrations ranging from about 846 pM to about 17 μM. Fluo-4 fluorescence is then measured for 5 minutes. 0.1 mL KCl agonist dissolved in A7r5 assay buffer is then added to the cells to produce a final concentration of 90 mM KCl, and fluorescence was measured for another 45 seconds. Data were collected over the entire time course and analyzed using Excel, Graph Pad Prism, or an in-house regression analysis software. Cloning of N- and L-type calcium channel subunit open reading frame cDNAs. Five cDNAs encoding subunits of the rat N- or L-type calcium channels were cloned by PCR amplification in order to reconstitute functional channels in a heterologous system. These were the alpha1b (α1b), beta1 (β1), beta3 (β3), alpha2delta (α2δ), and alpha1c (α1c) subunit cDNAs. The alpha1b subunit cDNA has been described by Dubel et al. in Proc. Natl. Acad. Sci. U.S.A 89: 5058-5062 (1992). The beta1 subunit cDNA has been described by Pragnell et al. in FEBS Lett. 291: 253-258 (1991). The beta3 subunit cDNA has been described by Castellano et al. in J. Biol. Chem. 268: 12359-12366 (1993). The alpha2delta subunit cDNA has been described by Kim et al. in Proc. Natl. Acad. Sci. U.S.A. 89: 3251-3255 (1992). The alphalc subunit cDNA has been described by Koch et al. in J. Biol. Chem. 265: 17786-17791 (1990).

The 7.0 kb cDNA containing the entire α1b open reading frame (ORF) was PCR amplified as two overlapping cDNA fragments, i.e., a 2.7 kb 5′ fragment and a 4.4 kb 3′ fragment. The 5′ fragment was amplified from rat brain cDNA using primers 1 (SEQ ID NO:1, TABLE 1) and 2 (SEQ ID NO:2, TABLE 1), and the 3′ fragment was amplified from rat spinal cord cDNA using primers 3 (SEQ ID NO:3, TABLE 1) and 4 (SEQ ID NO:4, TABLE 1). The two fragments were joined by ligation at a common restriction site to create the entire 7.0 kb cDNA. This ORF encodes the protein isoform generated by alternative splicing termed “+A ΔSFMG ΔET” according to the nomenclature of Lin et al. (Neuron 18: 153-166 (1997)). The entire cDNA was sequenced with redundant coverage on both strands. The cDNA was then inserted into the mammalian expression vector pcDNA6.2DEST (Invitrogen, Carlsbad Calif.) by homologous recombination using the Gateway system (Invitrogen).

The 1.8 kb cDNA encoding the β1 subunit, the 1.45 cDNA encoding the beta3 subunit, and the 3.3 kb cDNA encoding the alpha2delta subunit were cloned by PCR amplification from rat spinal cord cDNA (β1) or brain cDNA (β3, α2δ). Primers 5 (SEQ ID NO:5, TABLE 1) and 6 (SEQ ID NO:6, TABLE 1) were used for the β1 cDNA amplification; primers 7 (SEQ ID NO:7, TABLE 1) and 8 (SEQ ID NO:8, TABLE 1) were used for the β3 cDNA amplification; and primers 9 (SEQ ID NO:9, TABLE 1) and 10 (SEQ ID NO:10, TABLE 1) were used for the α2δ cDNA amplification. PCR products were subcloned and fully sequenced on both strands. Clones matching the reference sequence (β1: NM_{—}017346; β3: NM_{—}012828; α2δ: M86621) and the gene's GenBank rat genomic DNA sequences were recombined into the mammalian expression vector pcDNA3.2DEST (β1, β3) or pcDNA3.1-Zeo (α2δ), which had been modified to a vector compatible with the Gateway recombination system using the Gateway vector adaptor kit (Invitrogen). Proper recombination was confirmed by sequencing of recombinogenic regions. For β3 expression vector, proper protein expression was confirmed by Western blot analysis of lysates of transfected HEK293 cells using a rabbit polyclonal antiserum directed against the rat β3 subunit (USA Biological).

The 6.5 kb cDNA encoding the L-type calcium channel α1c subunit was cloned by PCR amplification from rat heart cDNA using primers 11 (SEQ ID NO:11, TABLE 1) and 12 (SEQ ID NO:12, TABLE 1). The PCR fragment was subcloned and fully sequenced on both strands to confirm its identity. A clone matching consensus reference sequence M59786 and rat genomic DNA sequences was recombined into the mammalian expression vector pcDNA6.2DEST. Sequences around the recombinogenic region were sequenced to confirm accurate recombination into the expression vector.

TABLE 1

| PRIMER SEQUENCE | SEQ ID NO. |
|---|---|
| CACC ATG GTC CGC TTC GGG GAC | 1 |
| CCG TTC AGT GGC CTC CTC C | 2 |
| C TAG CAC CAG TGA TCC TGG TCTG | 3 |
| AGT GCG TTG TGA GCG CAG TA | 4 |
| CAC CAT GGT CCA GAA GAG CGG | 5 |
| TCTCAGCGGATGTAGACGCCT | 6 |
| CAC CAT GTA TGA CGA CTC CTA C | 7 |
| GGT GGT CAG TAG CTG TCC TTA GG | 8 |
| CAC CAT GGC TGC TGG CTG CCT | 9 |
| AGA GGG TCA CCA TAG ATA GTG TCT G | 10 |
| CACCATGATTCGGGCCTTCGCT | 11 |
| AGCCTGCGGACTACAGGTTGCTGAC | 12 |

N-type Recombinant Cell Line Development. N-type calcium channel expressing HEK-293 cells were created in two stages. Stage 1 was created as follows. The rat α1b, and β3 cDNA expression constructs (2.5 μg each) were co-transfected into human embryonic kidney (HEK-293) cells by Lipofectamine Plus reagent (Invitrogen), as per manufacturer's instructions. 24 hours later, cells were split in limiting dilution into multiple 96-well plates in selection media containing 20 μg/mL blasticidin and 500 μg/mL geneticin, and incubated for 3 weeks at 37° C., 5% CO₂, 95% humidity. Plates containing ≦1 clone per well were cultured until wells positive for single clones were confluent. Individual clones were then arrayed into columns of a destination 96-well plate, and partly split into 6-well plates for culture maintenance. Array plates were washed once with IMR32 buffer and cells loaded for 1 hour with 0.1 mL of IMR32 buffer containing Fluo-4 (3 μM final concentration, Molecular Probes). Then they were washed twice with 0.1 mL of IMR32 buffer, and replaced with 0.1 mL IMR32 buffer. Plates were then transferred to a FLIPR⁹⁶ for assay. The FLIPR measured basal Fluo-4 fluorescence for 315 seconds, then added 0.1 mL KCl agonist dissolved in IMR32 buffer and measured fluorescence for another 45 seconds. Final KCl concentration was 90 mM. Data were collected over the entire time course and analyzed using Excel, Graph Pad Prism, or Activity Base (version 5.1, IDBS, Parsippany, N.J.) software. The clone with the greatest signal-to-noise ratio, best stability of response with passage number, and best adhesion to PDL precoated plates (Becton Dickinson) was expanded, characterized and used for stage 2 cell line development.

Stage 2 of N-type cell line development was carried out as follows. The rat α2δ cDNA expression construct (5 μg each) was transfected into the stage 1 N-type clonal cell line by Lipofectamine Plus reagent (Invitrogen), as per manufacturer's instructions. 24 hours later, cells were split in limiting dilution into multiple 96-well plates in selection media containing 20 μg/mL blasticidin, 500 μg/mL geneticin, and 250 μg/mL zeocin and incubated for 3 weeks at 37° C., 5% CO₂, 95% humidity. Plates containing ≦1 clone per well were cultured and handled according to the same steps and procedures described above for the stage 1 cell line. The three clones with the greatest signal-to-noise, best stability of response with passage number, and best adhesion to PDL precoated plates (Becton Dickinson) were expanded, characterized and tested in electrophysiology for the best current size, N-type pharmacology, N-type characteristic current-voltage relationship and kinetics as described below.

L-type Recombinant Cell Line Development. L-type calcium channel expressing HEK-293 cells were created in two stages. Stage 1 was created as follows. The rat α1c, and β1 cDNA expression constructs (2.5 μg each) were co-transfected into human embryonic kidney (HEK-293) cells by Lipofectamine Plus reagent (Invitrogen), as per manufacturer's instructions. 24 hours later, cells were split in limiting dilution into multiple 96-well plates in selection media containing 20 μg/mL blasticidin and 500 μg/mL geneticin, and incubated for 3 weeks at 37° C., 5% CO₂, 95% humidity. Plates containing ≦1 clone per well were cultured until wells positive for single clones were confluent. Individual clones were then arrayed into columns of a destination 96-well plate, and partly split into 6-well plates for culture maintenance. Array plates were washed once with LTCC wash (or assay) buffer and cells loaded for 1 hour with 0.1 mL of LTCC buffer containing Fluo-4 (3 μM final concentration, Molecular Probes). Then they were washed twice with 0.1 mL of LTCC buffer, and replaced with 0.1 mL LTCC buffer. Plates were then transferred to a FLIPR⁹⁶ for assay. The FLIPR measured basal Fluo-4 fluorescence for 315 seconds, then added 0.1 mL KCl agonist dissolved in LTCC buffer and measured fluorescence for another 45 seconds. Final KCl concentration was 90 mM. Data were collected over the entire time course and analyzed using Excel, Graph Pad Prism, or Activity Base software. The clone with the greatest signal-to-noise ratio, best stability of response with passage number, and best adhesion to PDL precoated plates (Becton Dickinson) was expanded, characterized and used for stage 2 cell line development.

Stage 2 of L-type cell line development was carried out as follows. The rat α2δ cDNA expression construct (5 μg each) was transfected into the stage 1 L-type clonal cell line by Lipofectamine Plus reagent (Invitrogen), as per manufacturer's instructions. 24 hours later, cells were split in limiting dilution into multiple 96-well plates in selection media containing 20 μg/mL blasticidin, 500 μg/mL geneticin, and 250 μg/mL zeocin and incubated for 3 weeks at 37° C., 5% CO₂, 95% humidity. Plates containing ≦1 clone per well were cultured and handled according to the same steps and procedures described above for the stage 1 cell line. The three clones with the greatest signal-to-noise, best stability of response with passage number, and best adhesion to PDL precoated plates (Becton Dickinson) were expanded and characterized.

N-type Electrophysiology in Recombinant Cells. For electrophysiological recording, the cells expressing α1b, β3 and α2δ subunits were seeded on 35-mm culture Petri dishes at a density of approximately 10⁴ cells/dish and kept in an incubator for up to three days for subsequent recordings. For recordings, the dishes were positioned on the stage of an inverted microscope (Nikon, Eclipse E600, Japan) and superfused with a bath solution comprised of BaCl₂ (11 mM), MgCl₂ (1.5 mM), HEPES (10 mM), TEA chloride (120 mM), glucose (10 mM) adjusted to pH 7.4 with KOH. Whole-cell voltage-clamp recordings were made using conventional patch-clamp techniques (Hamill et al., Pfluegers Arch. 391: 85-100 (1981)) at room temperature (22-24° C.). The patch-clamp pipettes were pulled from WPI, thick-walled borosilicate glass (WPI, Sarasota, Fla.). Currents were recorded using an Axopatch 200A amplifier (Axon Instruments, Union City, Calif.) and were leak-subtracted (P/4), low-pass filtered (1 kHz, 4-pole Bessel), digitized (20-50-μs intervals), and stored using Digidata 1200 B interface and Pclamp8.0/Clampex software (Axon Instruments, Union City, Calif.). The pipettes were back-filled with internal solution containing CsCl (110 mM), MgCl₂ (3 mM), EGTA (3 mM), HEPES (40 mM), Mg-ATP (4 mM), Na₂GTP (0.5 mM), and adjusted to pH 7.2 with CsOH. The pipette resistance ranged from 2 to 3 MOhm and was compensated by 75-80% by the built-in electronic circuitry.

Currents were elicited by stepping from a holding potential of −90 mV to 0 mV for 20 ms every 20 sec. At the −90 mV membrane voltage about 50% of channels were in the inactivated state, and thus contact with a blocker would involve interaction with both resting and inactivated channels. Every drug was applied at 3 to 4 concentrations increasing in a cumulative manner. Fractional inhibition levels in steady-state were used to draw the partial inhibition concentration curves to get the IC₅₀ (i.e. concentration causing 50% reduction in the size of the response) values at −90 mV.

Stock solutions of each test compound were prepared using DMSO. Serial dilutions to desired concentrations were done with bath solution; concentration of DMSO in final solutions was 0.1%. Drugs were applied by gravity flow using a plane multi-barrel array shooter positioned 0.5 mm apart from the cell.

All curve fittings were carried out using Origin software (version 5.0, Microcal). A Hill equation was fit to the concentration-inhibition curves to determine IC₅₀ values.

N-type Electrophysiology in Neuronal Cells. To determine dissociation constants in resting versus inactivated state for N-type calcium channels, neuronal cells that endogenously express N-type calcium channels can be used. For electrophysiological recording, the neuronal cells expressing N-type calcium channels are seeded on 35-mm culture Petri dishes at a density of approximately 10⁴ cells/dish and kept in an incubator for up to three days for subsequent recordings. For recordings, the dishes are positioned on the stage of an inverted microscope (Nikon, Eclipse E600, Japan) and superfused with a bath solution comprised of BaCl₂ (11 mM), MgCl₂ (1.5 mM), HEPES (10 mM), TEA chloride (120 mM), glucose (10 mM) adjusted to pH 7.4 with KOH. Whole-cell voltage-clamp recordings are made using conventional patch-clamp techniques (Hamill et al., Pfluegers Arch. 391: 85-100 (1981)) at room temperature (22-24° C.). The patch-clamp pipettes are pulled from WPI, thick-walled borosilicate glass (WPI, Sarasota, Fla.). Currents are recorded using an Axopatch 200A amplifier (Axon Instruments, Union City, Calif.) and leak-subtracted (P/4), low-pass filtered (1 kHz, 4-pole Bessel), digitized (20-50-μs intervals), and stored using Digidata 1200 B interface and Pclamp8.0/Clampex software (Axon Instruments, Union City, Calif.). The pipettes are back-filled with internal solution containing CsCl (110 mM), MgCl₂ (3 mM), EGTA (3 mM), HEPES (40 mM), Mg-ATP (4 mM), Na₂GTP (0.5 mM), and adjusted to pH 7.2 with CsOH. The pipette resistance ranges from 2 to 3 MOhm and is compensated by 75-80% by the built-in electronic circuitry.

Currents are elicited by stepping from a holding potential of −90 mV to 0 mV for 20 ms every 10 sec. At the −90 mV membrane voltage a proportion of channels is in the inactivated state, and thus contact with a blocker would involve interaction with both resting and inactivated channels. This protocol is used as a first tier screen. For dissection of two components of inhibition (resting block with the apparent dissociation constant Kᵣ and inactivated state block with Kᵢ), steady-state inactivation curves are collected using a double-pulse protocol. Three-second long depolarizing pre-pulse incrementing in 10 mV steps is followed by a 10 ms test pulse to 0 mV.

Stock solutions of each test compound are prepared using DMSO. Serial dilutions to desired concentrations are done with bath solution; concentration of DMSO in final solutions is 0.1%. Drugs are applied by gravity flow using a plane multi-barrel array shooter positioned ˜1 mm apart from the cell.

All curve fittings can be carried out using Origin software (version 5.0, Microcal). A Hill equation is used to fit the concentration-response curves and to determine IC₅₀ values. A Boltzman equation is used to fit inactivation curves, returning half-inactivation voltage, V_{0.5}, slope p and the amplitude of current at the most negative voltage where eventually all channels are in the resting state. These parameters are used to calculate the apparent dissociation constants: Kᵣ=((Ab/Ac)/(1−(Ab/Ac))*[b]) where [b] is the drug concentration, Ac is the maximum test current amplitude in control conditions and Ab is the maximum test current amplitude in the presence of a blocker; Kᵢ=[b]/((exp(−(dx/p))*(1+([b]/Kᵣ))−1) where dx is the difference between half-inactivation voltage V_{0.5} in the presence and absence of drug and p is the slope.

In vivo Pharmacology

The compounds of the present invention can be tested for in vivo anticonvulsant activity after i.v., p.o., or i.p. injection using any of a number of anticonvulsant tests in mice, including the maximum electroshock seizure test (MES). Maximum electroshock seizures are induced in male NSA mice weighing between 15-20 g and in male Sprague-Dawley rats weighing between 200-225 g by application of current (for mice: 50 mA, 60 pulses/sec, 0.8 msec pulse width, 1 sec duration, D.C.; for rats: 99 mA, 125 pulses/sec, 0.8 msec pulse width, 2 sec duration, D.C.) using a Ugo Basile ECT device (Model 7801). Mice are restrained by gripping the loose skin on their dorsal surface and saline-coated corneal electrodes are held lightly against the two corneae. Rats are allowed free movement on the bench top and ear-clip electrodes are used. Current is applied and animals are observed for a period of up to 30 seconds for the occurrence of a tonic hindlimb extensor response. A tonic seizure is defined as a hindlimb extension in excess of 90 degrees from the plane of the body. Results can be treated in a quantal manner.

The compounds can be tested for their antinociceptive activity in the formalin model as described in Hunskaar, S., O. B. Fasmer, and K. Hole, J. Neurosci. Methods 14: 69-76 (1985). Male Swiss Webster NIH mice (20-30 g; Harlan, San Diego, Calif.) can be used in all experiments. Food is withdrawn on the day of experiment. Mice are placed in Plexiglass jars for at least 1 hour to acclimate to the environment. Following the acclimation period mice are weighed and given either the compound of interest administered i.p. or p.o., or the appropriate volume of vehicle (10% Tween-80) as control. Fifteen minutes after the i.p. dosing, and 30 minutes after the p.o. dosing mice are injected with formalin (20 μL of 5% formaldehyde solution in saline) into the dorsal surface of the right hind paw. Mice are transferred to the Plexiglass jars and monitored for the amount of time spent licking or biting the injected paw. Periods of licking and biting are recorded in 5-minute intervals for 1 hour after the formalin injection. All experiments are done in a blinded manner during the light cycle. The early phase of the formalin response is measured as licking/biting between 0-5 minutes, and the late phase is measured from 15-50 minutes. Differences between vehicle and drug treated groups can be analyzed by one-way analysis of variance (ANOVA). A P value <0.05 is considered significant. Compounds are considered to be efficacious for treating acute and chronic pain if they have activity in blocking both the early and second phase of formalin-induced paw-licking activity.

Compounds can be tested for their potential to treat chronic pain (i.e., antiallodynic and antihyperalgesic activities) using the Chung model of peripheral neuropathy (Kim and Chung, Pain 50: 355-363 (1992)). Male Sprague-Dawley rats weighing between 200-225 g are anesthetized with halothane (1-3% in a mixture of 70% air and 30% oxygen), and their body temperature controlled during anesthesia through use of a homeothermic blanket. A 2-cm dorsal midline incision is then made at the L5 and L6 level, and the para-vertebral muscle groups retracted bilaterally. L5 and L6 spinal nerves are then exposed, isolated, and tightly ligated with 6-0 or 7-0 silk suture. A sham operation is performed exposing the contralateral L5 and L6 spinal nerves, without ligating, as a negative control.

Tactile Allodynia: Sensitivity to non-noxious mechanical stimuli can be measured in animals to assess tactile allodynia. Rats are transferred to an elevated testing cage with a wire mesh floor and allowed to acclimate for five to ten minutes. A series of von Frey monofilaments are applied to the plantar surface of the hindpaw to determine the animal's withdrawal threshold. The first filament used possesses a buckling weight of 9.1 gms (.96 log value) and is applied up to five times to see if it elicits a withdrawal response. If the animal has a withdrawal response, then the next lightest filament in the series would be applied up to five times to determine if it also could elicit a response. This procedure is repeated with subsequent lesser filaments until there is no response and the identity of the lightest filament that elicits a response is recorded. If the animal does not have a withdrawal response from the initial 9.1 gms filament, then subsequent filaments of increased weight are applied until a filament elicits a response and the identity of this filament is recorded. For each animal, three measurements are made at every time point to produce an average withdrawal threshold determination. Tests can be performed prior to, and at 1, 2, 4 and 24 hours post drug administration.

Mechanical Hyperalgesia: Sensitivity to noxious mechanical stimuli can be measured in animals using the paw pressure test to assess mechanical hyperalgesia. In rats, hind paw withdrawal thresholds (“PWT”), measured in grams, in response to a noxious mechanical stimulus are determined using an analgesymeter (Model 7200, commercially available from Ugo Basile of Italy), as described in Stein (Biochemistry & Behavior 31: 451-455 (1988)). The rat's paw is placed on a small platform, and weight is applied in a graded manner up to a maximum of 250 grams. The endpoint is taken as the weight at which the paw is completely withdrawn. PWT is determined once for each rat at each time point. PWT can be measured only in the injured paw, or in both the injured and non-injured paw. In one non-limiting embodiment, mechanical hyperalgesia associated with nerve injuty induced pain (neuropathic pain) can be assessed in rats. Rats are tested prior to surgery to determine a baseline, or normal, PWT. Rats are tested again 2 to 3 weeks post-surgery, prior to, and at different times after (e.g. 1, 3, 5 and 24 hr) drug administration. An increase in PWT following drug administration indicates that the test compound reduces mechanical hyperalgesia.

### Pharmaceutical Compositions

Although a compound of the present invention may be administered to a mammal in the form of a raw chemical without any other components present, the compound is preferably administered as part of a pharmaceutical composition containing the compound combined with a suitable pharmaceutically acceptable carrier. Such a carrier can be selected from pharmaceutically acceptable excipients and auxiliaries.

Compositions within the scope of the present invention include all compositions where a compound of the present invention is combined with a pharmaceutically acceptable carrier. In a preferred embodiment, the compound is present in the composition in an amount that is effective to achieve its intended therapeutic purpose. While individual needs may vary, a determination of optimal ranges of effective amounts of each compound is within the skill of the art. Typically, the compounds may be administered to mammal, e.g. human, orally at a dose of from about 0.0025 to about 1500 mg per kg body weight of the mammal, or an equivalent amount of a pharmaceutically acceptable salt thereof, per day to treat the particular disorder. A useful oral dose of a compound of the present invention administered to a mammal is from about 0.0025 to about 50 mg per kg body weight of the mammal, or an equivalent amount of the pharmaceutically acceptable salt thereof. For intramuscular injection, the dose is typically about one-half of the oral dose.

A unit oral dose may comprise from about 0.01 to about 50 mg, and preferably about 0.1 to about 10 mg, of the compound. The unit dose can be administered one or more times daily as one or more tablets, each containing from about 0.01 to about 50 mg of the compound, or an equivalent amount of a pharmaceutically acceptable salt or solvate thereof.

In one embodiment, a pharmaceutical composition of the present invention can be administered orally and is formulated into tablets, dragees, capsules or an oral liquid preparation.

Alternatively, a pharmaceutical composition of the present invention can be administered rectally, and is formulated in suppositories.

Alternatively, a pharmaceutical composition of the present invention can be administered by injection.

Alternatively, a pharmaceutical composition of the present invention can be administered transdermally.

Alternatively, a pharmaceutical composition of the present invention can be administered by inhalation or by intranasal administration.

Alternatively, a pharmaceutical composition of the present invention can be administered by the intravaginal route.

A pharmaceutical composition of the present invention can contain from about 0.01 to 99 percent by weight, and preferably from about 0.25 to 75 percent by weight, of active compound(s).

The present methods of the invention, such as the method for treating, preventing, or ameliorating a disorder responsive to the blockade of calcium channels in an animal in need thereof, can further comprise administering a second therapeutic agent to the animal being administered a compound of Formula I, I′, or I″. In one embodiment, the second therapeutic agent is administered in an effective amount.

Effective amounts of the other therapeutic agents are known to those skilled in the art. However, it is well within the skilled artisan's purview to determine the second therapeutic agent's optimal effective-amount range. In one embodiment of the invention, where a second therapeutic agent is administered to an animal, the effective amount of the compound of the present invention (i.e., the first therapeutic agent) is less than its effective amount would be where the second therapeutic agent is not administered. In this case, without being bound by theory, it is believed that compounds of the present invention and the second therapeutic agent act synergistically to treat, prevent, or ameliorate a disorder or condition.

The second therapeutic agent can be, but is not limited to, an opioid agonist, a non-opioid analgesic, a non-steroidal anti-inflammatory agent, an antimigraine agent, a Cox-II inhibitor, a β-adrenergic blocker, an anticonvulsant, an antidepressant, an anticancer agent, an agent for treating addictive disorder, an agent for treating Parkinson's disease and parkinsonism, an agent for treating anxiety, an agent for treating epilepsy, an agent for treating a seizure, an agent for treating a stroke, an agent for treating a pruritic condition, an agent for treating psychosis, an agent for treating ALS, an agent for treating a cognitive disorder, an agent for treating a migraine, an agent for treating vomiting, an agent for treating dyskinesia, or an agent for treating depression, and mixtures thereof.

Examples of useful opioid agonists include, but are not limited to, alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, sufentanil, tilidine, tramadol, pharmaceutically acceptable salts thereof, and mixtures thereof.

In certain embodiments, the opioid agonist is selected from codeine, hydromorphone, hydrocodone, oxycodone, dihydrocodeine, dihydromorphine, morphine, tramadol, oxymorphone, pharmaceutically acceptable salts thereof, and mixtures thereof.

Examples of useful non-opioid analgesics include non-steroidal anti-inflammatory agents, such as aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, isoxicam, and pharmaceutically acceptable salts thereof, and mixtures thereof. Examples of other suitable non-opioid analgesics include the following, non limiting, chemical classes of analgesic, antipyretic, nonsteroidal antiinflammatory drugs: salicylic acid derivatives, including aspirin, sodium salicylate, choline magnesium trisalicylate, salsalate, diflunisal, salicylsalicylic acid, sulfasalazine, and olsalazin; para aminophennol derivatives including acetaminophen and phenacetin; indole and indene acetic acids, including indomethacin, sulindac, and etodolac; heteroaryl acetic acids, including tolmetin, diclofenac, and ketorolac; anthranilic acids (fenamates), including mefenamic acid, and meclofenamic acid; enolic acids, including oxicams (piroxicam, tenoxicam), and pyrazolidinediones (phenylbutazone, oxyphenthartazone); and alkanones, including nabumetone. For a more detailed description of the NSAIDs, see Paul A. Insel, Analgesic Antipyretic and Antiinflammatory Agents and Drugs Employed in the Treatment of Gout, in Goodman & Gilman's The Pharmacological Basis of Therapeutics 617-57 (Perry B. Molinhoff and Raymond W. Ruddon eds., 9th ed 1996) and Glen R. Hanson, Analgesic, Antipyretic and Anti Inflammatory Drugs in Remington: The Science and Practice of Pharmacy Vol II 1196-1221 (A. R. Gennaro ed. 19th ed. 1995) which are hereby incorporated by reference in their entireties. Suitable Cox-II inhibitors and 5-lipoxygenase inhibitors, as well as combinations thereof, are described in U.S. Pat. No. 6,136,839, which is hereby incorporated by reference in its entirety. Examples of useful Cox II inhibitors include, but are not limited to, rofecoxib and celecoxib.

Examples of useful antimigraine agents include, but are not limited to, alpiropride, bromocriptine, dihydroergotamine, dolasetron, ergocornine, ergocorninine, ergocryptine, ergonovine, ergot, ergotamine, flumedroxone acetate, fonazine, ketanserin, lisuride, lomerizine, methylergonovine, methysergide, metoprolol, naratriptan, oxetorone, pizotyline, propranolol, risperidone, rizatriptan, sumatriptan, timolol, trazodone, zolmitriptan, and mixtures thereof.

Examples of useful β-adrenergic blockers include, but are not limited to, acebutolol, alprenolol, amosulabol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol, indenolol, labetalol, levobunolol, mepindolol, metipranolol, metoprolol, moprolol, nadolol, nadoxolol, nebivalol, nifenalol, nipradilol, oxprenolol, penbutolol, pindolol, practolol, pronethalol, propranolol, sotalol, sulfinalol, talinolol, tertatolol, tilisolol, timolol, toliprolol, and xibenolol.

Examples of useful anticonvulsants include, but are not limited to, acetylpheneturide, albutoin, aloxidone, aminoglutethimide, 4-amino-3-hydroxybutyric acid, atrolactamide, beclamide, buramate, calcium bromide, carbamazepine, cinromide, clomethiazole, clonazepam, decimemide, diethadione, dimethadione, doxenitroin, eterobarb, ethadione, ethosuximide, ethotoin, felbamate, fluoresone, gabapentin, 5-hydroxytryptophan, lamotrigine, magnesium bromide, magnesium sulfate, mephenytoin, mephobarbital, metharbital, methetoin, methsuximide, 5-methyl-5-(3-phenanthryl)-hydantoin, 3-methyl-5-phenylhydantoin, narcobarbital, nimetazepam, nitrazepam, oxcarbazepine, paramethadione, phenacemide, phenetharbital, pheneturide, phenobarbital, phensuximide, phenylmethylbarbituric acid, phenytoin, phethenylate sodium, potassium bromide, pregabaline, primidone, progabide, sodium bromide, solanum, strontium bromide, suclofenide, sulthiame, tetrantoin, tiagabine, topiramate, trimethadione, valproic acid, valpromide, vigabatrin, and zonisamide.

Examples of useful antidepressants include, but are not limited to, binedaline, caroxazone, citalopram, (S)-citalopram, dimethazan, fencamine, indalpine, indeloxazine hydrocholoride, nefopam, nomifensine, oxitriptan, oxypertine, paroxetine, sertraline, thiazesim, trazodone, benmoxine, iproclozide, iproniazid, isocarboxazid, nialamide, octamoxin, phenelzine, cotinine, rolicyprine, rolipram, maprotiline, metralindole, mianserin, mirtazepine, adinazolam, amitriptyline, amitriptylinoxide, amoxapine, butriptyline, clomipramine, demexiptiline, desipramine, dibenzepin, dimetacrine, dothiepin, doxepin, fluacizine, imipramine, imipramine N-oxide, iprindole, lofepramine, melitracen, metapramine, nortriptyline, noxiptilin, opipramol, pizotyline, propizepine, protriptyline, quinupramine, tianeptine, trimipramine, adrafinil, benactyzine, bupropion, butacetin, dioxadrol, duloxetine, etoperidone, febarbamate, femoxetine, fenpentadiol, fluoxetine, fluvoxamine, hematoporphyrin, hypericin, levophacetoperane, medifoxamine, milnacipran, minaprine, moclobemide, nefazodone, oxaflozane, piberaline, prolintane, pyrisuccideanol, ritanserin, roxindole, rubidium chloride, sulpiride, tandospirone, thozalinone, tofenacin, toloxatone, tranylcypromine, L-tryptophan, venlafaxine, viloxazine, and zimeldine.

Examples of useful anticancer agents include, but are not limited to, acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, and cisplatin.

Therapeutic agents useful for treating or preventing an addictive disorder include, but are not limited to, methadone, desipramine, amantadine, fluoxetine, buprenorphine, an opiate agonist, 3-phenoxypyridine, or a serotonin antagonist.

Examples of useful therapeutic agents for treating or preventing Parkinson's disease and parkinsonism include, but are not limited to, carbidopa/levodopa, pergolide, bromocriptine, ropinirole, pramipexole, entacapone, tolcapone, selegiline, amantadine, and trihexyphenidyl hydrochloride.

Examples of useful therapeutic agents for treating or preventing anxiety include, but are not limited to, benzodiazepines, such as alprazolam, brotizolam, chlordiazepoxide, clobazam, clonazepam, clorazepate, demoxepam, diazepam, estazolam, flumazenil, flurazepam, halazepam, lorazepam, midazolam, nitrazepam, nordazepam, oxazepam, prazepam, quazepam, temazepam, and triazolam; non-benzodiazepine agents, such as buspirone, gepirone, ipsapirone, tiospirone, zolpicone, zolpidem, and zaleplon; tranquilizers, such as barbituates, e.g., amobarbital, aprobarbital, butabarbital, butalbital, mephobarbital, methohexital, pentobarbital, phenobarbital, secobarbital, and thiopental; and propanediol carbamates, such as meprobamate and tybamate.

Examples of useful therapeutic agents for treating or preventing epilepsy or seizure include, but are not limited to, carbamazepine, ethosuximide, gabapentin, lamotrigine, phenobarbital, phenytoin, primidone, valproic acid, trimethadione, benzodiazepines, gamma-vinyl GABA, acetazolamide, and felbamate.

Examples of useful therapeutic agents for treating or preventing stroke include, but are not limited to, anticoagulants such as heparin, agents that break up clots such as streptokinase or tissue plasminogen activator, agents that reduce swelling such as mannitol or corticosteroids, and acetylsalicylic acid.

Examples of useful therapeutic agents for treating or preventing a pruritic condition include, but are not limited to, naltrexone; nalmefene; danazol; tricyclics such as amitriptyline, imipramine, and doxepin; antidepressants such as those given below; menthol; camphor; phenol; pramoxine; capsaicin; tar; steroids; and antihistamines.

Examples of useful therapeutic agents for treating or preventing psychosis include, but are not limited to, phenothiazines such as chlorpromazine hydrochloride, mesoridazine besylate, and thoridazine hydrochloride; thioxanthenes such as chloroprothixene and thiothixene hydrochloride; clozapine; risperidone; olanzapine; quetiapine; quetiapine fumarate; haloperidol; haloperidol decanoate; loxapine succinate; molindone hydrochloride; pimozide; and ziprasidone.

Examples of useful therapeutic agents for treating or preventing ALS include, but are not limited to, baclofen, neurotrophic factors, riluzole, tizanidine, benzodiazepines such as clonazepan and dantrolene.

Examples of useful therapeutic agents for treating or preventing cognitive disorders include, but are not limited to, agents for treating or preventing dementia such as tacrine; donepezil; ibuprofen; antipsychotic drugs such as thioridazine and haloperidol; and antidepressant drugs such as those given below.

Examples of useful therapeutic agents for treating or preventing a migraine include, but are not limited to, sumatriptan; methysergide; ergotamine; caffeine; and beta-blockers such as propranolol, verapamil, and divalproex.

Examples of useful therapeutic agents for treating or preventing vomiting include, but are not limited to, 5-HT3 receptor antagonists such as odansetron, dolasetron, granisetron, and tropisetron; dopamine receptor antagonists such as prochlorperazine, thiethylperazine, chlorpromazine, metoclopramide, and domperidone; glucocorticoids such as dexamethasone; and benzodiazepines such as lorazepam and alprazolam.

Examples of useful therapeutic agents for treating or preventing dyskinesia include, but are not limited to, reserpine and tetrabenazine.

Examples of useful therapeutic agents for treating or preventing depression include, but are not limited to, tricyclic antidepressants such as amitryptyline, amoxapine, bupropion, clomipramine, desipramine, doxepin, imipramine, maprotiline, nefazadone, nortriptyline, protriptyline, trazodone, trimipramine, and venlafaxine; selective serotonin reuptake inhibitors such as citalopram, (S)-citalopram, fluoxetine, fluvoxamine, paroxetine, and setraline; monoamine oxidase inhibitors such as isocarboxazid, pargyline, phenelzine, and tranylcypromine; and psychostimulants such as dextroamphetamine and methylphenidate.

A compound of the present invention and the second therapeutic agent can act additively or, in one embodiment, synergistically. In one embodiment, a compound of the present invention is administered concurrently with the second therapeutic agent; for example, a composition comprising an effective amount of a compound of Formula I, I′, or I″, and an effective amount of a second therapeutic agent can be administered. Alternatively, a composition comprising an effective amount of a compound of Formula I, I′, or I″ and a different composition comprising an effective amount of a second therapeutic agent can be concurrently administered. In another embodiment, an effective amount of a compound of the present invention is administered prior or subsequent to administration of an effective amount of a second therapeutic agent. In this embodiment, the compound of the present invention is administered while the second therapeutic agent exerts its therapeutic effect, or the second therapeutic agent is administered while the compound of the present invention exerts its preventive or therapeutic effect for treating, ameliorating or preventing a disorder or condition.

A pharmaceutical composition of the present invention can be administered to any animal that may experience the beneficial effects of a compound of the present invention. Foremost among such animals are mammals, e.g., humans and companion animals, although the invention is not intended to be so limited.

A pharmaceutical composition of the present invention can be administered by any means that achieves its intended purpose. For example, administration can be by the parenteral, subcutaneous, intravenous, intramuscular, intraperitoneal, transdermal, intranasal, rectal, intravaginal or buccal route, or by inhalation. Alternatively, or concurrently, administration can be by the oral route. The dosage administered and route of administration will vary, depending upon the circumstances of the particular subject, and taking into account such factors as age, health, and weight of the recipient, condition or disorder to be treated, kind of concurrent treatment, if any, frequency of treatment, and the nature of the effect desired.

A pharmaceutical composition of the present invention is preferably manufactured in a manner which is itself known, for example, by means of conventional mixing, granulating, dragee-making, dissolving, extrusion, or lyophilizing processes. Thus, pharmaceutical compositions for oral use can be obtained by combining the active compound with solid excipients, optionally grinding the resulting mixture and processing the mixture of granules, after adding suitable auxiliaries, if desired or necessary, to obtain tablets or dragee cores.

Suitable excipients include fillers such as saccharides (for example, lactose, sucrose, mannitol or sorbitol), cellulose preparations, calcium phosphates (for example, tricalcium phosphate or calcium hydrogen phosphate), as well as binders such as starch paste (using, for example, maize starch, wheat starch, rice starch, or potato starch), gelatin, tragacanth, methyl cellulose, hydroxypropylmethylcellulose, sodium carboxymethylcellulose, and/or polyvinyl pyrrolidone. If desired, one or more disintegrating agents can be added, such as the above-mentioned starches and also carboxymethyl-starch, cross-linked polyvinyl pyrrolidone, agar, or alginic acid or a salt thereof, such as sodium alginate.

Auxiliaries are typically flow-regulating agents and lubricants such as, for example, silica, talc, stearic acid or salts thereof (e.g., magnesium stearate or calcium stearate), and polyethylene glycol. Dragee cores are provided with suitable coatings that are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropymethyl-cellulose phthalate can be used. Dye stuffs or pigments may be added to the tablets or dragee coatings, for example, for identification or in order to characterize combinations of active compound doses.

Examples of other pharmaceutical preparations that can be used orally include push-fit capsules made of gelatin, or soft, sealed capsules made of gelatin and a plasticizer such as glycerol or sorbitol. The push-fit capsules can contain a compound in the form of granules, which may be mixed with fillers such as lactose, binders such as starches, and/or lubricants such as talc or magnesium stearate and, optionally, stabilizers, or in the form of extruded multiparticulates. In soft capsules, the active compounds are preferably dissolved or suspended in suitable liquids, such as fatty oils or liquid paraffin. In addition, stabilizers may be added.

Possible pharmaceutical preparations for rectal administration include, for example, suppositories, which consist of a combination of one or more active compounds with a suppository base. Suitable suppository bases include natural and synthetic triglycerides, and paraffin hydrocarbons, among others. It is also possible to use gelatin rectal capsules consisting of a combination of active compound with a base material such as, for example, a liquid triglyceride, polyethylene glycol, or paraffin hydrocarbon.

Suitable formulations for parenteral administration include aqueous solutions of the active compound in a water-soluble form such as, for example, a water-soluble salt, alkaline solution, or acidic solution. Alternatively, a suspension of the active compound may be prepared as an oily suspension. Suitable lipophilic solvents or vehicles for such as suspension may include fatty oils (for example, sesame oil), synthetic fatty acid esters (for example, ethyl oleate), triglycerides, or a polyethylene glycol such as polyethylene glycol-400 (PEG-400). An aqueous suspension may contain one or more substances to increase the viscosity of the suspension, including, for example, sodium carboxymethyl cellulose, sorbitol, and/or dextran. The suspension may optionally contain stabilizers.

The following examples are illustrative, but not limiting, of the compounds, compositions and methods of the present invention. Suitable modifications and adaptations of the variety of conditions and parameters normally encountered in clinical therapy and which are obvious to those skilled in the art in view of this disclosure are within the spirit and scope of the invention.

### EXAMPLES

### Example 1

### N,N-Diethyl-2-[1-(4-trifluoromethoxybenzenesulfonyl)piperidin-4-ylideneaminooxy]acetamide

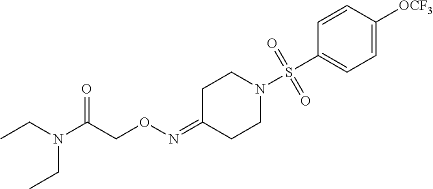

a) 4-(Trifluoromethoxy)benzenesulfonyl chloride (5.0 g, 19 mmol) was added to a solution of 4-piperidone monohydrate hydrochloride (3.0 g, 17 mmol) and triethylamine (6 ml, 82 mmol) in CH₂Cl₂ (40 ml) at 0° C. The reaction mixture was warmed to room temperature and stirred for 4 hours, washed with water (20 ml) and brine (15 ml), and concentrated under reduced pressure. The residue was purified by column chromatography on silica (EtOAc/Hexanes 30/70) to give 1-(4-(trifluoromethoxy)phenylsulfonyl)piperidin-4-one as a white solid (4.5 g, 76%, colorless solid).

b) A mixture of 1-(4-(trifluoromethoxy)phenylsulfonyl)piperidin-4-one (3.0 g, 9.2 mmol), hydroxylamine hydrochloride (1.2 g, 17 mmol) and sodium acetate (3.5 g, 42 mmol) in EtOH (40 ml) was stirred at room temperature for 16 hours. The solvent was removed under reduced pressure. The residue was suspended in CH₂Cl₂ (100 ml), washed with water and brine, and concentrated to give a white solid which was recrystallized from EtOH to give 1-(4-(trifluoromethoxy)phenylsulfonyl)piperidin-4-one oxime (2.0 g, 62%, colorless solid).

c) A mixture of 1-(4-(trifluoromethoxy)phenylsulfonyl)piperidin-4-one oxime (0.20 g, 0.59 mmol), 2-chloro-N,N-diethylacetamide (105 mg, 0.7 mmol), potassium carbonate (0.50 g, 3.6 mmol) and potassium iodide (20 mg, 0.12 mmol) in CH₃CN (2 ml) was stirred at 80° C. for 14 hours. After cooling to room temperature, the reaction mixture was diluted with EtOAc (15 ml), washed with water (4 ml) and brine (4 ml) and evaporated under reduced pressure. The residue was purified by column chromatography on silica (EtOAc/Hexanes 30/70-50/50) to give N,N-diethyl-2-[1-(4-trifluoromethoxybenzenesulfonyl)piperidin-4-ylideneaminooxy]acetamide (110 mg, 50%, colorless solid, LC: 100%, retention time: 8.73 min (Method B), M/Z: 452 [M+H]⁺).

¹H-NMR (CDCl₃) δ: 7.82 (2H, d, 8.9 Hz), 7.36 (2H, dd, 0.9, 8.1 Hz), 4.65 (2H, s), 336 (2H, dd, 7.0, 7.2 Hz), 3.31 (2H, t, 5.5 Hz), 3.18-3.24 (6H, m), 2.8 (2H, dd, 5.9, 6.3 Hz), 2.45 (2H, dd, 6.1, 5.4 Hz), 1.16 (3H, t, 7.2 Hz), 1.10 (3H, t, 7.2 Hz).

Similarly, the following compounds were prepared:

### Example 2

### N-Phenyl-2-[1-(4-trifluoromethoxybenzenesulfonyl)piperidin-4-ylideneaminooxy]acetamide

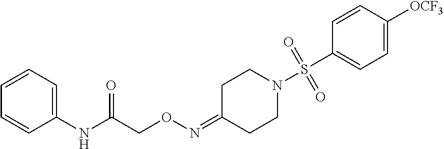

Colorless solid, LC: 100%, retention time: 9.17 min (Method B), M/Z: 472 [M+H]⁺.

¹H-NMR (CDCl₃) δ: 7.84 (2H, d, 8.8 Hz), 7.75 (1H, br, NH), 7.47-7.51 (2H, m), 7.38 (2H, d, 8.1 Hz), 7.36 (2H, dd, 7.4, 8.1 Hz), 7.14 (1H, dd, 7.2, 7.4 Hz), 4.56 (2H, s), 3.23-3.29 (4H, m), 2.83 (2H, t, 6.1 Hz), 2.51 (2H, t, 5.9 Hz).

### Example 3

### N-Methyl-N-phenyl-2-[1-(4-trifluoromethoxybenzenesulfonyl)piperidin-4-ylideneaminooxy]acetamide

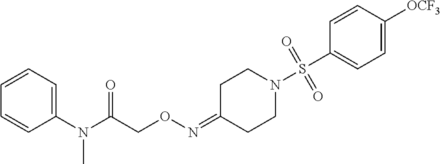

Colorless solid, LC: 100%, retention time: 9.21 min (Method B), M/Z: 486 [M+H]⁺.

¹H-NMR (CDCl₃) δ: 7.81 (2H, d, 8.8 Hz), 7.47-7.51 (2H, m), 7.34-7.32 (5H, m), 7.17-7.19 (2H, m), 4.35 (2H, s), 3.26 (3H, s), 3.14-3.22 (4H, m), 2.71 (2H, dd, 5.9, 6.1 Hz), 2.41 (2H, dd, 5.7, 6.1 Hz).

### Example 4

### N-(4-Fluorophenyl)-2-[1-(4-trifluoromethoxybenzenesulfonyl)piperidin-4-ylideneaminooxy]acetamide

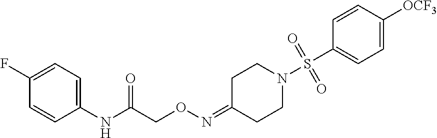

Colorless solid, LC: 98%, retention time: 9.23 min (Method B), M/Z: 490 [M+H]⁺.

¹H-NMR (CDCl₃) δ: 7.82 (2H, d, 8.8 Hz), 7.72 (1H, br, NH), 7.44-7.48 (2H, m), 7.36-7.39 (2H, m), 7.00-7.04 (2H, m), 4.56 (2H, s), 3.22-3.29 (4H, m), 2.83 (2H, t, 6.1 Hz), 2.51 (2H, t, 5.9 Hz).

### Example 5

### N-(Thiazol-2-yl)-2-(1-(3-(trifluoromethyl)phenylsulfonyl)piperidin-4-ylideneaminooxy)acetamide

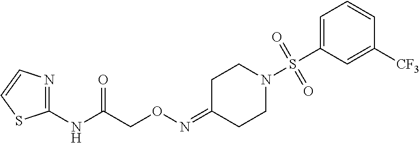

a) (Aminooxy)acetic acid hemihydrochloride (6.05 g, 55.4 mmol) was added to the solution of 1-(3-(trifluoromethyl)phenylsulfonyl)piperidin-4-one (6.79 g, 22.1 mmol) in pyridine (40 ml) and stirred at room temperature for 67 hours. Hydrochloric acid (4 M, 120 ml) was added dropwisely to the mixture, the precipitated material was collected, washed with hydrochloric acid (2 M, 20 ml×3), dissolved in CHCl₃, washed with brine (50 ml), dried (Na₂SO₄) and evaporated under reduced pressure. The resulted crude material was triturated with Et₂O/diisopropyl ether (1/1) to give 2-(1-(3-(trifluoromethyl)phenylsulfonyl)piperidin-4-ylideneaminooxy)acetic acid (6.31 g, 75%, colorless solid).

b) A solution of 2-(1-(3-(trifluoromethyl)phenylsulfonyl)piperidin-4-ylideneaminooxy)acetic acid (19.0 mg, 0.048 mmol) and O-(benzotriazol-1-yl)-N,N,N′,N′-tetramethyluronium hexafluorophosphate (27.3 mg, 0.072 mmol) in DMF (1 ml) and triethylamine (0.030 ml) was added to thiazol-2-amine (2.0 mg, 0.02 mmol) at room temperature. The reaction mixture was shaken at room temperature for 16 hours, quenched with water (1.0 ml), extracted with EtOAc (1.0 ml×2), and evaporated under reduced pressure. The residue was dissolved in DMSO (0.8 ml) and purified by HPLC to give N-(thiazol-2-yl)-2-(1-(3-(trifluoromethyl)phenylsulfonyl)piperidin-4-ylideneaminooxy)acetamide (LC: 100%, retention time: 1.95 min (Method C), M/Z: 462.9 [M+H]⁺).

Similarly, the following compounds were prepared:

### Example 6

### N-Isopropyl-2-(1-(3-(trifluoromethyl)phenylsulfonyl)piperidin-4-ylideneaminooxy)acetamide

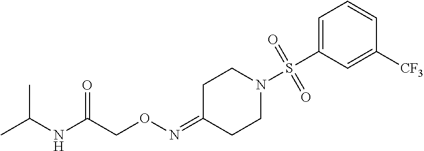

LC: 100%, retention time: 1.90 min (Method C), M/Z: 421.9 [M+H]⁺.

### Example 7

### N-(6-Methoxypyridin-3-yl)-2-(1-(3-(trifluoromethyl)phenylsulfonyl)piperidin-4-ylideneaminooxy)acetamide

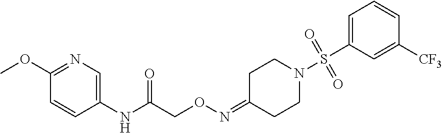

LC: 100%, retention time: 1.94 min (Method C), M/Z: 486.9 [M+H]⁺.

### Example 8

### N-(4-Fluorophenyl)-2-(1-(3-(trifluoromethyl)phenylsulfonyl)piperidin-4-ylideneaminooxy)acetamide

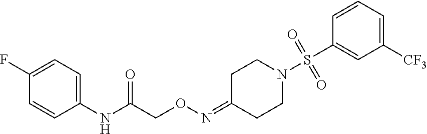

LC: 100%, retention time: 2.16 min (Method C), M/Z: 473.9 [M+H]⁺.

### Example 9

### N-Cyclopropyl-2-(1-(3-(trifluoromethyl)phenylsulfonyl)piperidin-4-ylideneaminooxy)acetamide

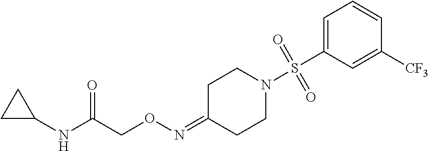

LC: 100%, retention time: 1.78 min (Method C), M/Z: 420.0 [M+H]⁺.

### Example 10

### N-(2-Cyanoethyl)-2-(1-(3-(trifluoromethyl)phenylsulfonyl)piperidin-4-ylideneaminooxy)acetamide

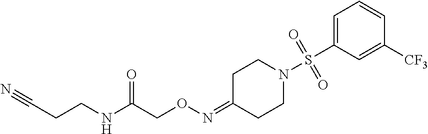

LC: 100%, retention time: 1.73 min (Method C), M/Z: 433.0 [M+H]⁺.

### Example 11

### N-(3-Chlorophenyl)-2-(1-(3-(trifluoromethyl)phenylsulfonyl)piperidin-4-ylideneaminooxy)acetamide

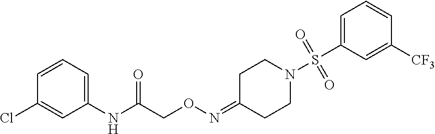

LC: 100%, retention time: 2.30 min (Method C), M/Z: 489.9 [M+H]⁺.

### Example 12

### N-(Pyridin-4-yl)-2-(1-(3-(trifluoromethyl)phenylsulfonyl)piperidin-4-ylideneaminooxy)acetamide

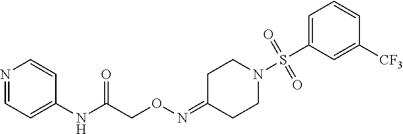

LC: 100%, retention time: 1.26 min (Method C), M/Z: 458.1 [M+H]⁺.

### Example 13

### N-(Pyridin-2-yl)-2-(1-(3-(trifluoromethyl)phenylsulfonyl)piperidin-4-ylideneaminooxy)acetamide

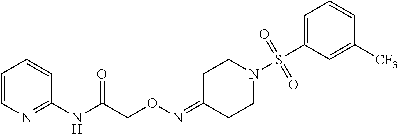

LC: 100%, retention time: 1.87 min (Method C), M/Z: 457.0 [M+H]⁺.

### Example 14

### N-Benzyl-N-methyl-2-(1-(3-(trifluoromethyl)phenylsulfonyl)piperidin-4-ylideneaminooxy)acetamide

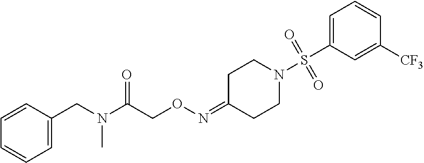

LC: 100%, retention time: 2.16 min (Method C), M/Z: 484.4 [M+H]⁺.

### Example 15

### N-(2-Methoxyphenyl)-2-(1-(3-(trifluoromethyl)phenylsulfonyl)piperidin-4-ylideneaminooxy)acetamide

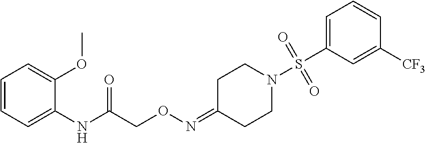

LC: 100%, retention time: 2.25 min (Method C), M/Z: 486.0 [M+H]⁺.

### Example 16

### N-(2,2,2-Trifluoroethyl)-2-(1-(3-(trifluoromethyl)phenylsulfonyl)piperidin-4-ylideneaminooxy)acetamide

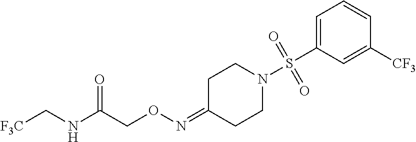

LC: 100%, retention time: 1.99 min (Method C), M/Z: 436.0 [M+H]⁺.

### Example 17

### N-(2-Hydroxyethyl)-N-phenyl-2-(1-(3-(trifluoromethyl)phenylsulfonyl)piperidin-4-ylideneaminooxy)acetamide

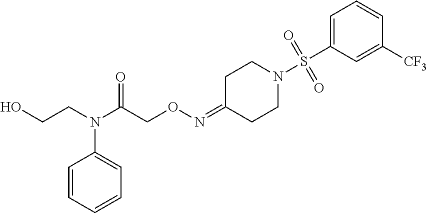

LC: 96%, retention time: 2.28 min (Method C), M/Z: 500.1 [M+H]⁺.

### Example 18

### N-(4-Acetylphenyl)-2-(1-(3-(trifluoromethyl)phenylsulfonyl)piperidin-4-ylideneaminooxy)acetamide

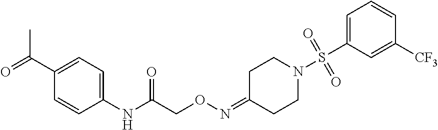

LC: 100%, retention time: 2.05 min (Method C), M/Z: 497.9 [M+H]⁺.

### Example 19

### N-Cyano-2-(1-(3-(trifluoromethyl)phenylsulfonyl)piperidin-4-ylideneaminooxy)acetamide

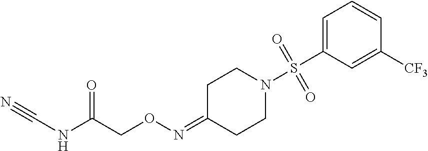

LC: 94%, retention time: 1.81 min (Method C), M/Z: 405.0 [M+H]⁺.

### Example 20

### N-(Pyridin-3-yl)-2-(1-(3-(trifluoromethyl)phenylsulfonyl)piperidin-4-ylideneaminooxy)acetamide

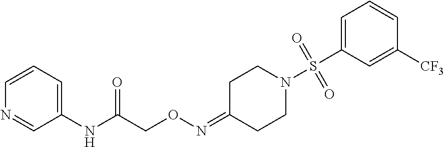

LC: 100%, retention time: 1.35 min (Method C), M/Z: 458.2 [M+H]⁺.

### Example 21

### N-tert-Butyl-2-(1-(3-(trifluoromethyl)phenylsulfonyl)piperidin-4-ylideneaminooxy)acetamide

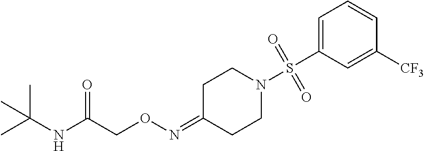

LC: 100%, retention time: 2.06 min (Method C), M/Z: 436.0 [M+H]⁺.

### Example 22

### N-(1-Phenylethyl)-2-(1-(3-(trifluoromethyl)phenylsulfonyl)piperidin-4-ylideneaminooxy)acetamide

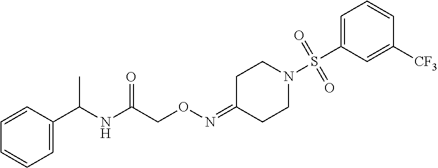

LC: 100%, retention time: 2.16 min (Method C), M/Z: 484.0 [M+H]⁺.

### Example 23

### N-(4-Fluorobenzyl)-2-(1-(3-(trifluoromethyl)phenylsulfonyl)piperidin-4-ylideneaminooxy)acetamide

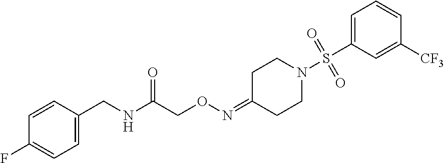

LC: 100%, retention time: 2.08 min (Method C), M/Z: 488.0 [M+H]⁺.

### Example 24

### 1-(3,4-Dihydroquinolin-1(2H)-yl)-2-(1-(3-(trifluoromethyl)phenylsulfonyl)piperidin-4-ylideneaminooxy)ethanone

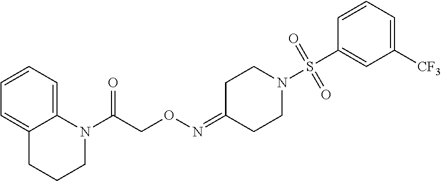

LC: 100%, retention time: 2.28 min (Method C), M/Z: 496.1 [M+H]⁺.

### Example 25

### N-Cyclohexyl-2-(1-(3-(trifluoromethyl)phenylsulfonyl)piperidin-4-ylideneaminooxy)acetamide

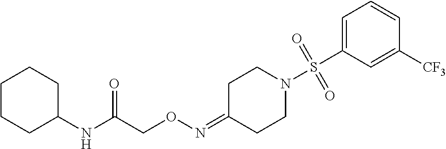

LC: 100%, retention time: 2.13 min (Method C), M/Z: 462.1 [M+H]⁺.

### Example 26

### N-Phenyl-2-(1-(3-(trifluoromethyl)phenylsulfonyl)piperidin-4-ylideneaminooxy)acetamide

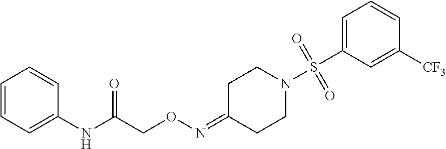

LC: 100%, retention time: 2.14 min (Method C), M/Z: 456.1 [M+H]⁺.

### Example 27

### N-(2-Phenoxyethyl)-2-(1-(3-(trifluoromethyl)phenylsulfonyl)piperidin-4-ylideneaminooxy)acetamide

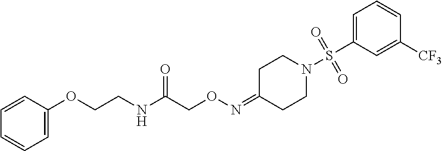

LC: 100%, retention time: 2.15 min (Method C), M/Z: 500.1 [M+H]⁺.

### Example 28

### N-(2-Methoxy-6-methylphenyl)-2-(1-(3-(trifluoromethyl)phenylsulfonyl)piperidin-4-ylideneaminooxy)acetamide

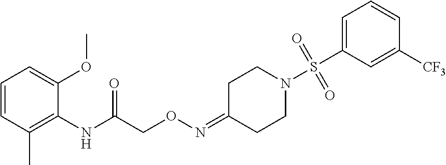

LC: 100%, retention time: 2.09 min (Method C), M/Z: 500.1 [M+H]⁺.

### Example 29

### N-(Pyridin-2-ylmethyl)-2-(1-(3-(trifluoromethyl)phenylsulfonyl)piperidin-4-ylideneaminooxy)acetamide

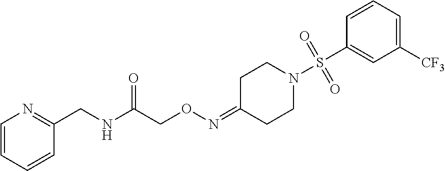

LC: 100%, retention time: 1.36 min (Method C), M/Z: 471.1 [M+H]⁺.

### Example 30

### 1-(5,6-Dihydropyridin-1(2H)-yl)-2-(1-(3-(trifluoromethyl)phenylsulfonyl)piperidin-4-ylideneaminooxy)ethanone

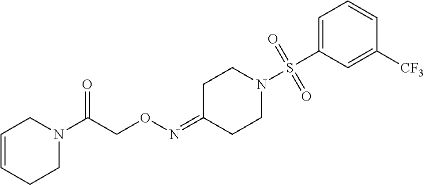

LC: 100%, retention time: 1.95 min (Method C), M/Z: 446.0 [M+H]⁺.

### Example 31

### N-(4-Methoxyphenyl)-2-(1-(3-(trifluoromethyl)phenylsulfonyl)piperidin-4-ylideneaminooxy)acetamide

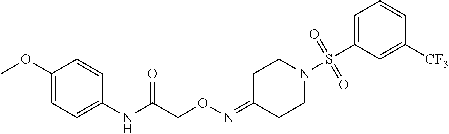

LC: 100%, retention time: 2.10 min (Method C), M/Z: 486.0 [M+H]⁺.

### Example 32

### 2-(1-(3-(Trifluoromethyl)phenylsulfonyl)piperidin-4-ylideneaminooxy)-N-(2,4,4-trimethylpentan-2-yl)acetamide

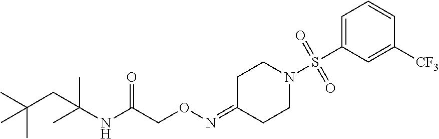

LC: 94%, retention time: 2.46 min (Method C), M/Z: 492.2 [M+H]⁺.

### Example 33

### N-(1,3,4-Thiadiazol-2-yl)-2-(1-(3-(trifluoromethyl)phenylsulfonyl)piperidin-4-ylideneaminooxy)acetamide

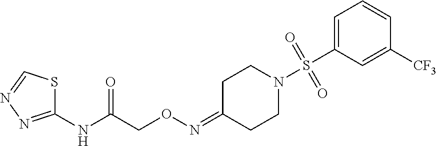

LC: 100%, retention time: 1.76 min (Method C), M/Z: 464.0 [M+H]⁺.

### Example 34

### N-(2-Cyanoethyl)-N-methyl-2-(1-(3-(trifluoromethyl)phenylsulfonyl)piperidin-4-ylideneaminooxy)acetamide

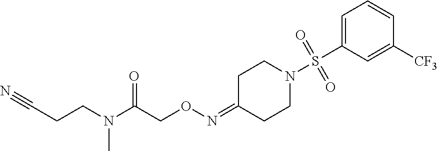

LC: 100%, retention time: 1.75 min (Method C), M/Z: 447.1 [M+H]⁺.

### Example 35

### N-(2,3-Dihydro-1H-inden-1-yl)-2-(1-(3-(trifluoromethyl)phenylsulfonyl)piperidin-4-ylideneaminooxy)acetamide

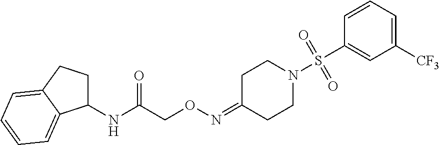

LC: 100%, retention time: 2.18 min (Method C), M/Z: 496.1 [M+H]⁺.

### Example 36

### N-(3-Methoxybenzyl)-2-(1-(3-(trifluoromethyl)phenylsulfonyl)piperidin-4-ylideneaminooxy)acetamide

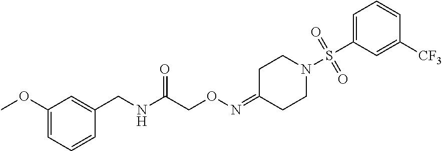

LC: 100%, retention time: 2.08 min (Method C), M/Z: 500.1 [M+H]⁺.

### Example 37

### N-(3-Methylbenzyl)-2-(1-(3-(trifluoromethyl)phenylsulfonyl)piperidin-4-ylideneaminooxy)acetamide

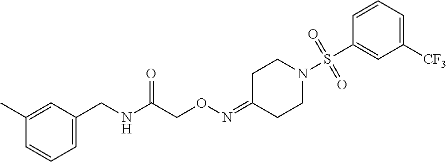

LC: 100%, retention time: 2.16 min (Method C), M/Z: 484.1 [M+H]⁺.

### Example 38

### 1-(Indolin-1-yl)-2-(1-(3-(trifluoromethyl)phenylsulfonyl)piperidin-4-ylideneaminooxy)ethanone

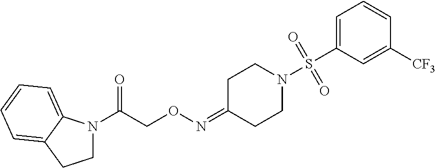

LC: 100%, retention time: 2.25 min (Method C), M/Z: 482.1 [M+H]⁺.

### Example 39

### N-(2,3-Dihydro-1H-inden-2-yl)-2-(1-(3-(trifluoromethyl)phenylsulfonyl)piperidin-4-ylideneaminooxy)acetamide

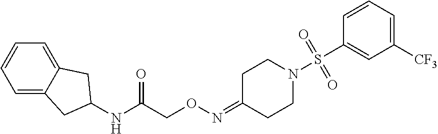

LC: 100%, retention time: 2.19 min (Method C), M/Z: 496.0 [M+H]⁺.

### Example 40

### N-(Pyridin-4-ylmethyl)-2-(1-(3-(trifluoromethyl)phenylsulfonyl)piperidin-4-ylideneaminooxy)acetamide

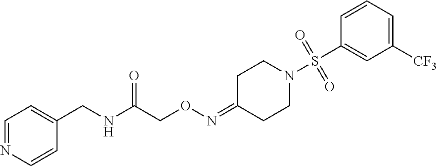

LC: 100%, retention time: 1.17 min (Method C), M/Z: 471.5 [M+H]⁺.

### Example 41

### N-(Pyridin-3-ylmethyl)-2-(1-(3-(trifluoromethyl)phenylsulfonyl)piperidin-4-ylideneaminooxy)acetamide

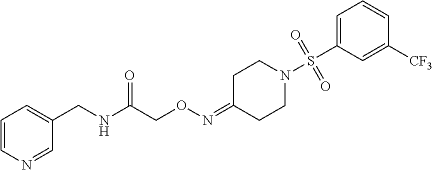

LC: 100%, retention time: 1.23 min (Method C), M/Z: 472.2 [M+H]⁺.

### Example 42

### N-(2-Morpholinoethyl)-2-(1-(3-(trifluoromethyl)phenylsulfonyl)piperidin-4-ylideneaminooxy)acetamide

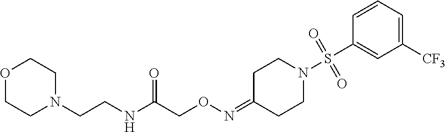

LC: 100%, retention time: 1.16 min (Method C), M/Z: 493.5 [M+H]⁺.

### Example 43

### N-Butyl-2-(1-(3-(trifluoromethyl)phenylsulfonyl)piperidin-4-ylideneaminooxy)acetamide

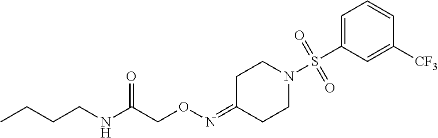

LC: 100%, retention time: 1.97 min (Method C), M/Z: 436.0 [M+H]⁺.

### Example 44

### N-Cyclohexyl-2-(2-(1-(3-(trifluoromethyl)phenylsulfonyl)piperidin-4-ylideneaminooxy)acetamido)benzamide

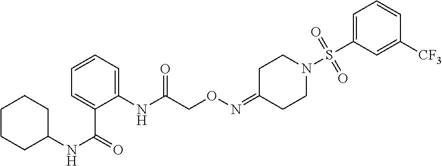

LC: 100%, retention time: 2.51 min (Method C), M/Z: 581.1 [M+H]⁺.

### Example 45

### 1-(2-Methylpiperidin-1-yl)-2-(1-(3-(trifluoromethyl)phenylsulfonyl)piperidin-4-ylideneaminooxy)ethanone

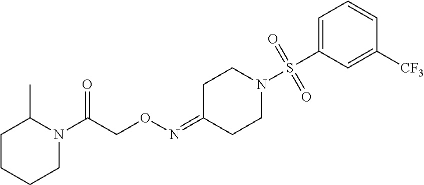

LC: 100%, retention time: 1.98 min (Method C), M/Z: 462.0 [M+H]⁺.

### Example 46

### 1-(3-Hydroxypiperidin-1-yl)-2-(1-(3-(trifluoromethyl)phenylsulfonyl)piperidin-4-ylideneaminooxy)ethanone

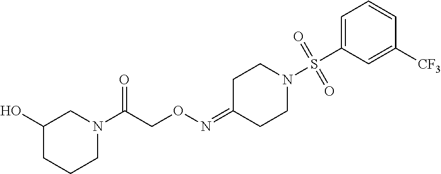

LC: 100%, retention time: 1.61 min (Method C), M/Z: 464.0 [M+H]⁺.

### Example 47

### 1-(4-Hydroxypiperidin-1-yl)-2-(1-(3-(trifluoromethyl)phenylsulfonyl)piperidin-4-ylideneaminooxy)ethanone

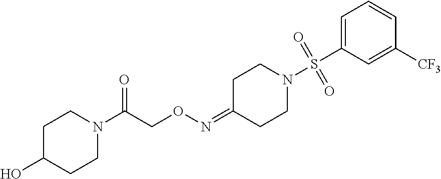

LC: 100%, retention time: 1.57 min (Method C), M/Z: 464.0 [M+H]⁺.

### Example 48

### 1-(3,5-Dimethylpiperidin-1-yl)-2-(1-(3-(trifluoromethyl)phenylsulfonyl)piperidin-4-ylideneaminooxy)ethanone

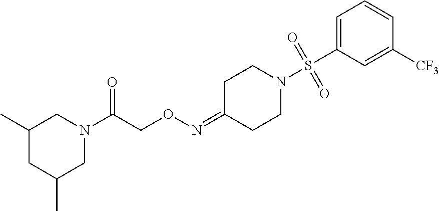

LC: 100%, retention time: 2.27 min (Method C), M/Z: 476.4 [M+H]⁺.

### Example 49

### 1-(4-(Hydroxymethyl)piperidin-1-yl)-2-(1-(3-(trifluoromethyl)phenylsulfonyl)piperidin-4-ylideneaminooxy)ethanone

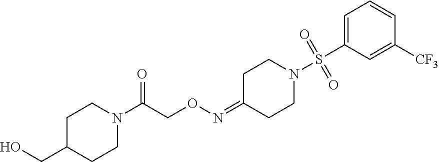

LC: 100%, retention time: 1.63 min (Method C), M/Z: 478.1 [M+H]⁺.

### Example 50

### 1-(3-Hydroxypyrrolidin-1-yl)-2-(1-(3-(trifluoromethyl)phenylsulfonyl)piperidin-4-ylideneaminooxy)ethanone

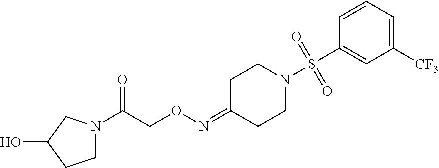

LC: 100%, retention time: 1.50 min (Method C), M/Z: 450.0 [M+H]⁺.

### Example 51

### N-(1H-Indol-5-yl)-2-(1-(3-(trifluoromethyl)phenylsulfonyl)piperidin-4-ylideneaminooxy)acetamide

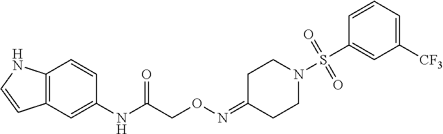

LC: 100%, retention time: 1.99 min (Method C), M/Z: 495.1 [M+H]⁺.

### Example 52

### N-Benzyl-2-(1-(3-(trifluoromethyl)phenylsulfonyl)piperidin-4-ylideneaminooxy)acetamide

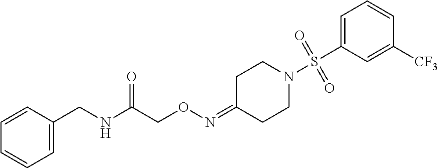

LC: 100%, retention time: 2.05 min (Method C), M/Z: 470.1 [M+H]⁺.

### Example 53

### N-(4-Cyanophenyl)-2-(1-(3-(trifluoromethyl)phenylsulfonyl)piperidin-4-ylideneaminooxy)acetamide

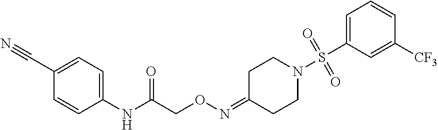

LC: 100%, retention time: 2.11 min (Method C), M/Z: 481.1 [M+H]⁺.

### Example 54

### N-(3-Fluorobenzyl)-2-(1-(3-(trifluoromethyl)phenylsulfonyl)piperidin-4-ylideneaminooxy)acetamide

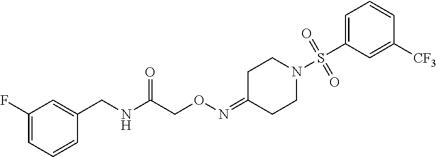

LC: 100%, retention time: 2.09 min (Method C), M/Z: 488.1 [M+H]⁺.

### Example 55

### 1-Morpholino-2-(1-(3-(trifluoromethyl)phenylsulfonyl)piperidin-4-ylideneaminooxy)ethanone

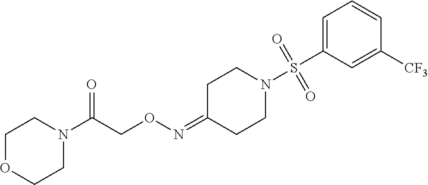

LC: 100%, retention time: 1.71 min (Method C), M/Z: 450.2 [M+H]⁺.

### Example 56

### N-(4-Chlorophenyl)-2-(1-(3-(trifluoromethyl)phenylsulfonyl)piperidin-4-ylideneaminooxy)acetamide

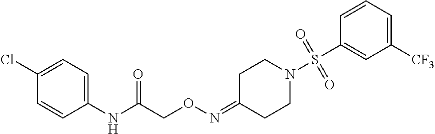

LC: 100%, retention time: 2.29 min (Method C), M/Z: 490.0 [M+H]⁺.

### Example 57

### N-(1-Hydroxypropan-2-yl)-2-(1-(3-(trifluoromethyl)phenylsulfonyl)piperidin-4-ylideneaminooxy)acetamide

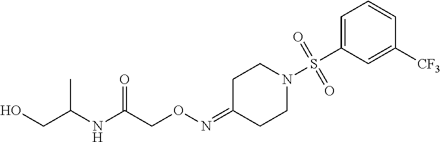

LC: 100%, retention time: 1.59 min (Method C), M/Z: 438.1 [M+H]⁺.

### Example 58

### N-(2-Fluoroethyl)-2-(1-(3-(trifluoromethyl)phenylsulfonyl)piperidin-4-ylideneaminooxy)acetamide

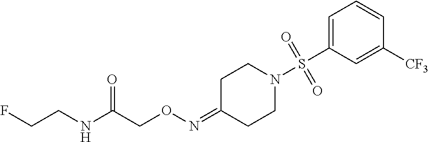

LC: 100%, retention time: 1.78 min (Method C), M/Z: 426.1 [M+H]⁺.

### Example 59

### N-(3,3-Dimethylbutyl)-2-(1-(3-(trifluoromethyl)phenylsulfonyl)piperidin-4-ylideneaminooxy)acetamide

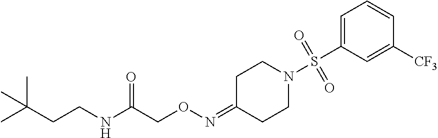

LC: 100%, retention time: 2.23 min (Method C), M/Z: 464.2 [M+H]⁺.

### Example 60

### N-Methyl-N-propyl-2-(1-(3-(trifluoromethyl)phenylsulfonyl)piperidin-4-ylideneaminooxy)acetamide

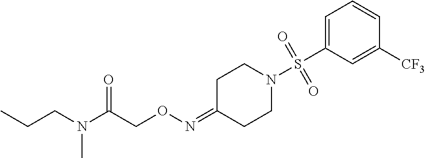

LC: 100%, retention time: 1.99 min (Method C), M/Z: 436.2 [M+H]⁺.

### Example 61

### N-(2-Hydroxyethyl)-N-methyl-2-(1-(3-(trifluoromethyl)phenylsulfonyl)piperidin-4-ylideneaminooxy)acetamide

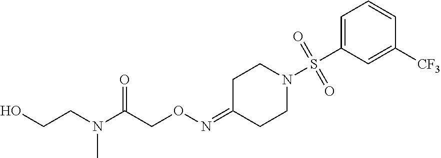

LC: 100%, retention time: 1.53 min (Method C), M/Z: 438.1 [M+H]⁺.

### Example 62

### N-(1-Hydroxy-2-methylpropan-2-yl)-2-(1-(3-(trifluoromethyl)phenylsulfonyl)piperidin-4-ylideneaminooxy)acetamide

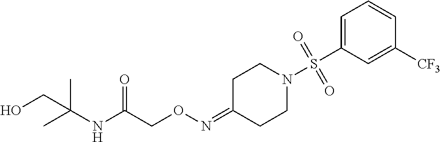

LC: 100%, retention time: 1.76 min (Method C), M/Z: 452.1 [M+H]⁺.

### Example 63

### N-Propyl-2-(1-(3-(trifluoromethyl)phenylsulfonyl)piperidin-4-ylideneaminooxy)acetamide

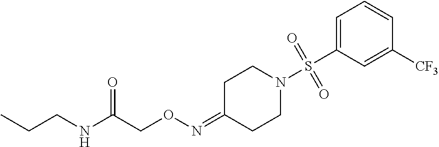

LC: 100%, retention time: 1.89 min (Method C), M/Z: 422.1 [M+H]⁺.

### Example 64

### N,N-Diisopropyl-2-(1-(3-(trifluoromethyl)phenylsulfonyl)piperidin-4-ylideneaminooxy)acetamide

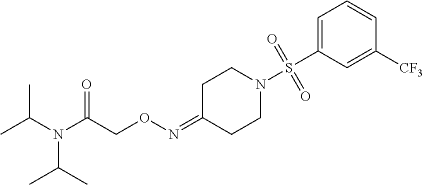

LC: 100%, retention time: 2.26 min (Method C), M/Z: 464.1 [M+H]⁺.

### Example 65

### N-sec-Butyl-2-(1-(3-trifluoromethyl)phenylsulfonyl)piperidin-4-ylideneaminooxy)acetamide

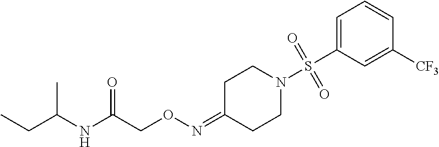

LC: 100%, retention time: 1.98 min (Method C), M/Z: 436.1 [M+H]⁺.

### Example 66

### N-(Pentan-3-yl)-2-(1-(3-(trifluoromethyl)phenylsulfonyl)piperidin-4-ylideneaminooxy)acetamide

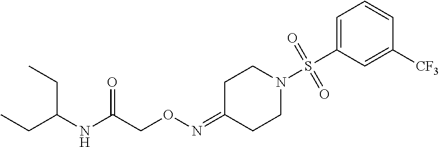

LC: 100%, retention time: 2.12 min (Method C), M/Z: 450.1 [M+H]⁺.

### Example 67

### N-Neopentyl-2-(1-(3-(trifluoromethyl)phenylsulfonyl)piperidin-4-ylideneaminooxy)acetamide

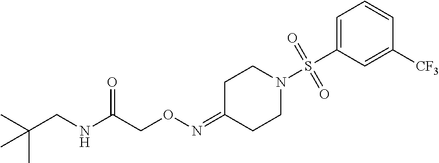

LC: 100%, retention time: 2.10 min (Method C), M/Z: 450.2 [M+H]⁺.

### Example 68

### N-Isopentyl-2-(1-(3-(trifluoromethyl)phenylsulfonyl)piperidin-4-ylideneaminooxy)acetamide

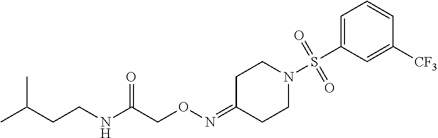

LC: 100%, retention time: 2.16 min (Method C), M/Z: 450.1 [M+H]⁺.

### Example 69

### N-Isobutyl-N-methyl-2-(1-(3-(trifluoromethyl)phenylsulfonyl)piperidin-4-ylideneaminooxy)acetamide

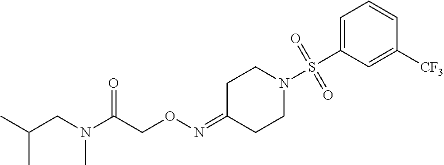

LC: 100%, retention time: 2.09 min (Method C), M/Z: 450.1 [M+H]⁺.

### Example 70

### N-(2-Hydroxybutyl)-2-(1-(3-(trifluoromethyl)phenylsulfonyl)piperidin-4-ylideneaminooxy)acetamide

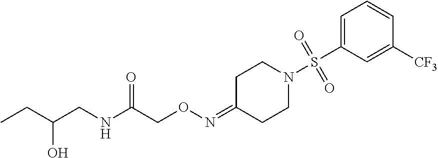

LC: 100%, retention time: 1.72 min (Method C), M/Z: 452.1 [M+H]⁺.

### Example 71

### N-(3-Hydroxy-2,2-dimethylpropyl)-2-(1-(3-(trifluoromethyl)phenylsulfonyl)piperidin-4-ylideneaminooxy)acetamide

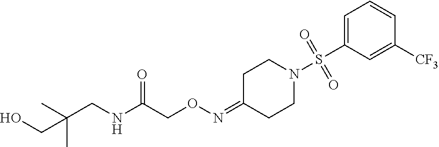

LC: 100%, retention time: 1.79 min (Method C), M/Z: 466.1 [M+H]⁺.

### Example 72

### N-(2-Methoxyethyl)-2-(1-(3-(trifluoromethyl)phenylsulfonyl)piperidin-4-ylideneaminooxy)acetamide

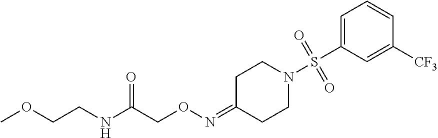

LC: 100%, retention time: 1.75 min (Method C), M/Z: 438.2 [M+H]⁺.

### Example 73

### N-(3,3-Dimethylbutan-2-yl)-2-(1-(3-(trifluoromethyl)phenylsulfonyl)piperidin-4-ylideneaminooxy)acetamide

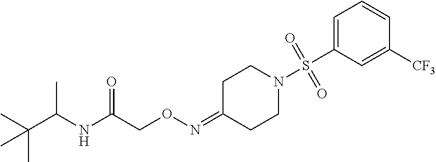

LC: 100%, retention time: 2.18 min (Method C), M/Z: 464.2 [M+H]⁺.

### Example 74

### N-(2-Hydroxypropyl)-2-(1-(3-(trifluoromethyl)phenylsulfonyl)piperidin-4-ylideneaminooxy)acetamide

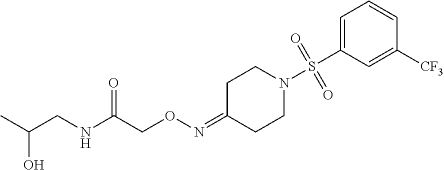

LC: 100%, retention time: 1.61 min (Method C), M/Z: 438.1 [M+H]⁺.

### Example 75

### N-Isobutyl-2-(1-(3-(trifluoromethyl)phenylsulfonyl)piperidin-4-ylideneaminooxy)acetamide

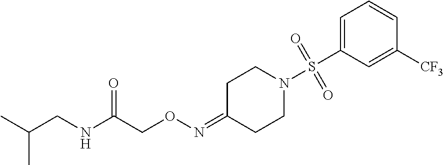

LC: 100%, retention time: 2.00 min (Method C), M/Z: 436.2 [M+H]⁺.

### Example 76

### N-(3-Hydroxypropyl)-2-(1-(3-(trifluoromethyl)phenylsulfonyl)piperidin-4-ylideneaminooxy)acetamide

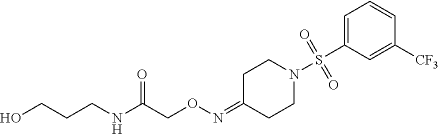

LC: 100%, retention time: 1.58 min (Method C), M/Z: 438.1 [M+H]⁺.

### Example 77

### N-(Cyanomethyl)-2-(1-(3-(trifluoromethyl)phenylsulfonyl)piperidin-4-ylideneaminooxy)acetamide

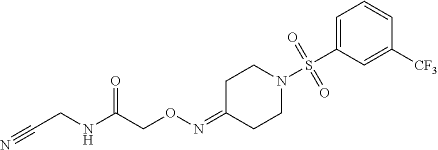

LC: 100%, retention time: 1.74 min (Method C), M/Z: 419.1 [M+H]⁺.

### Example 78

### N-(2-Fluorophenyl)-2-(1-(3-(trifluoromethyl)phenylsulfonyl)piperidin-4-ylideneaminooxy)acetamide

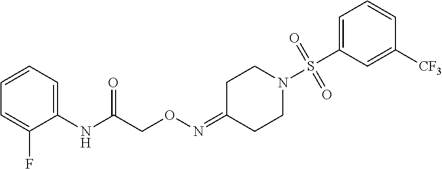

LC: 100%, retention time: 2.21 min (Method C), M/Z: 474.1 [M+H]⁺.

### Example 79

### N-(4-Hydroxycyclohexyl)-2-(1-(3-(trifluoromethyl)phenylsulfonyl)piperidin-4-ylideneaminooxy)acetamide

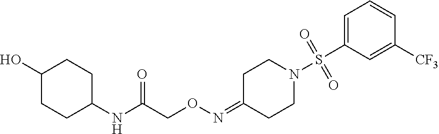

LC: 100%, retention time: 1.61 min (Method C), M/Z: 478.2 [M+H]⁺.

### Example 80

### N′,N′-Dimethyl-2-(1-(3-(trifluoromethyl)phenylsulfonyl)piperidin-4-ylideneaminooxy)acetohydrazide

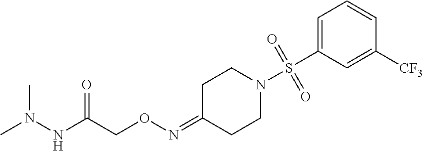

LC: 90%, retention time: 1.56 min (Method C), M/Z: 423.2 [M+H]⁺.

### Example 81

### N-(Pyrimidin-4-yl)-2-(1-(3-(trifluoromethyl)phenylsulfonyl)piperidin-4-ylideneaminooxy)acetamide

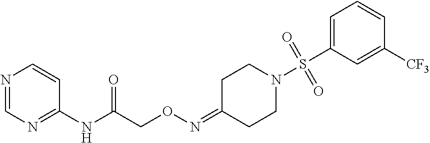

LC: 100%, retention time: 1.75 min (Method C), M/Z: 458.2 [M+H]⁺.

### Example 82

### N-Cyclobutyl-2-(1-(3-(trifluoromethyl)phenylsulfonyl)piperidin-4-ylideneaminooxy)acetamide

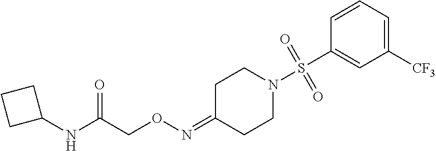

LC: 100%, retention time: 1.97 min (Method C), M/Z: 436.2 [M+H]⁺.

### Example 83

### N-(3-Cyanophenyl)-2-(1-(3-(trifluoromethyl)phenylsulfonyl)piperidin-4-ylideneaminooxy)acetamide

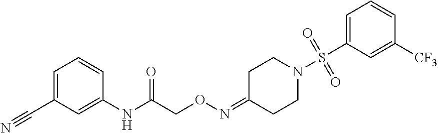

LC: 100%, retention time: 2.10 min (Method C), M/Z: 481.1 [M+H]⁺.

### Example 84

### N-(3-Fluorophenyl)-2-(1-(3-(trifluoromethyl)phenylsulfonyl)piperidin-4-ylideneaminooxy)acetamide

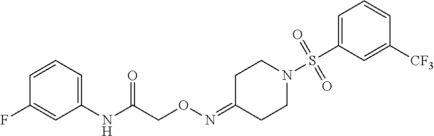

LC: 100%, retention time: 2.20 min (Method C), M/Z: 474.1 [M+H]⁺.

### Example 85

### N-(3-Methoxyphenyl)-2-(1-(3-(trifluoromethyl)phenylsulfonyl)piperidin-4-ylideneaminooxy)acetamide

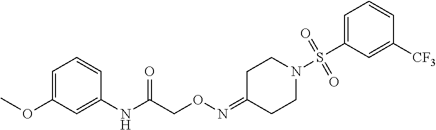

LC: 100%, retention time: 2.15 min (Method C), M/Z: 486.0 [M+H]⁺.

### Example 86

### N-((Tetrahydro-2H-pyran-4-yl)methyl)-2-(1-(3-(trifluoromethyl)phenylsulfonyl)piperidin-4-ylideneaminooxy)acetamide

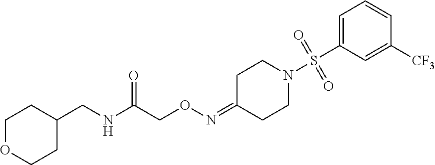

LC: 100%, retention time: 2.08 min (Method C), M/Z: 477.2 [M+H]⁺.

### Example 87

### N-(5-Methylhexan-2-yl)-2-(1-(3-(trifluoromethyl)phenylsulfonyl)piperidin-4-ylideneaminooxy)acetamide

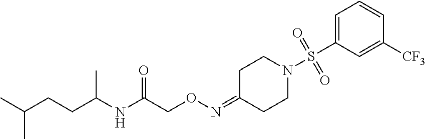

LC: 100%, retention time: 1.78 min (Method C), M/Z: 478.3 [M+H]⁺.

### Example 88

### N-(2,3-Dihydrobenzofuran-5-yl)-2-(1-(3-(trifluoromethyl)phenylsulfonyl)piperidin-4-ylideneaminooxy)acetamide

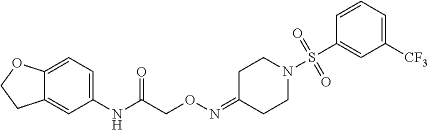

LC: 100%, retention time: 2.08 min (Method C), M/Z: 498.2 [M+H]⁺.

### Example 89

### N-(Thiazol-2-ylmethyl)-2-(1-(3-(trifluoromethyl)phenylsulfonyl)piperidin-4-ylideneaminooxy)acetamide

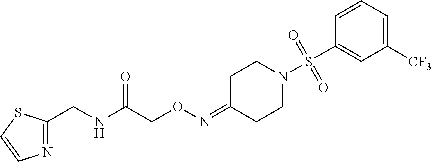

LC: 100%, retention time: 1.99 min (Method C), M/Z: 476.2 [M+H]⁺.

### Example 90

### 1-(Isoindolin-2-yl)-2-(1-(3-(trifluoromethyl)phenylsulfonyl)piperidin-4-ylideneaminooxy)ethanone

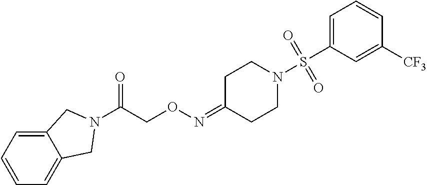

LC: 82%, retention time: 2.08 min (Method C), M/Z: 482.2 [M+H]⁺.

### Example 91

### N-(1-Methoxypropan-2-yl)-2-(1-(3-(trifluoromethyl)phenylsulfonyl)piperidin-4-ylideneaminooxy)acetamide

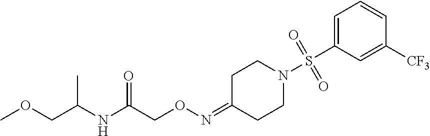

LC: 100%, retention time: 1.83 min (Method C), M/Z: 452.3 [M+H]⁺.

### Example 92

### N-Ethyl-2-(1-(3-(trifluoromethyl)phenylsulfonyl)piperidin-4-ylideneaminooxy)acetamide

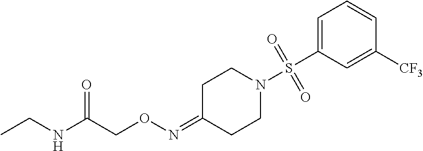

LC: 82%, retention time: 1.79 min (Method C), M/Z: 408.2 [M+H]⁺.

### Example 93

### N-(6-Fluoropyridin-3-yl)-2-(1-(3-(trifluoromethyl)phenylsulfonyl)piperidin-4-ylideneaminooxy)acetamide

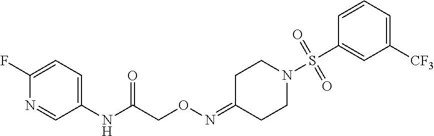

LC: 100%, retention time: 1.95 min (Method C), M/Z: 475.2 [M+H]⁺.

### Example 94

### 1-(4-Trifluoromethoxybenzenesulfonyl)piperidin-4-one O-(2-oxo-2-pyrrolidin-1-ylethyl)oxime

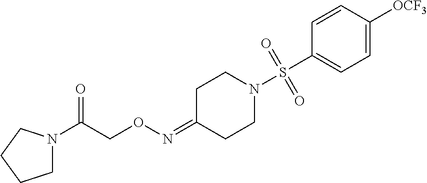

N-(3-Dimethylamino-1-propyl)-N′-ethylcarbodiimide hydrochloride (105 mg, 0.55 mmol) was added to a solution of 2-(1-(4-(trifluoromethoxy)phenylsulfonyl)piperidin-4-ylideneaminooxy)acetic acid (200 mg, 0.50 mmol), pyrrolidine (0.042 ml 0.50 mmol), 1-hydroxybenzotriazole (20 mg, 0.15 mmol) and 4-dimethylaminopyridine (6.1 mg, 0.050 mol) in CH₂Cl₂ (3 ml) and stirred at room temperature for 24 hours. The precipitated materials were filtered off and the filtrate was evaporated under reduced pressure. The residue was purified by column chromatography on silica (THF/CH₂Cl₂ 10/90-25/75) to give 1-(4-Trifluoromethoxybenzenesulfonyl)piperidin-4-one O-(2-oxo-2-pyrrolidin-1-ylethyl)oxime (150 mg, 65%, colorless solid, LC: 100%, retention time: 8.29 min (Method B), M/Z: 450 [M+H]⁺).

¹H-NMR (CDCl₃) δ: 7.82 (2H, d, 9.0 Hz), 7.36 (2H, dd, 0.9, 8.1 Hz), 4.57 (2H, s), 3.47 (2H, t, 6.8 Hz), 3.36 (2H, t, 6.7 Hz), 3.30 (2H, t, 5.8 Hz), 3.19 (2H, t, 6.1 Hz), 2.81 (2H, t, 5.9 Hz), 2.45 (2H, t, 5.7 Hz), 1.92-1.98 (2H, m), 1.80-1.86 (2H, m).

Similarly, the following compounds were prepared:

### Example 95

### N-Methyl-2-[1-(4-trifluoromethoxybenzenesulfonyl)piperidin-4-ylideneaminooxy]acetamide

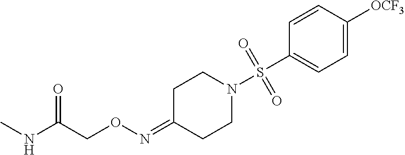

Colorless solid, LC: 100%, retention time: 7.70 min (Method B), M/Z: 410 [M+H]⁺.

¹H-NMR (CDCl₃) δ: 7.82-7.86 (2H, m), 7.36-7.39 (2H, m), 6.06 (1H, br, NH), 4.45 (2H, s), 3.25 (2H, t, 5.9 Hz), 3.2 (2H, t, 6.1 Hz), 2.86 (3H, d, 5.0 Hz), 2.76 (2H, t, 5.9 Hz), 2.48 (2H, t, 6.2 Hz).

### Example 96

### N,N-Dimethyl-2-[1-(4-trifluoromethoxybenzenesulfonyl)piperidin-4-ylideneaminooxy]acetamide

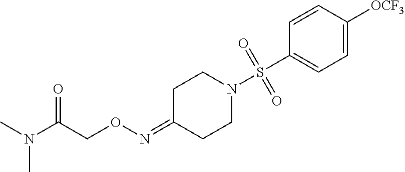

Colorless solid, LC: 100%, retention time: 7.85 min (Method B), M/Z: 424 [M+H]⁺.

¹H-NMR (CDCl₃) δ: 7.82 (2H, d, 9.0 Hz), 7.36 (2H, dd, 0.9, 8.1 Hz), 4.65 (2H, s), 3.23 (2H, t, 5.9 Hz), 3.19 (2H, t, 6.4 Hz), 2.94 (3H, s), 2.92 (3H, s), 2.80 (2H, t, 5.9 Hz), 2.45 (2H, t, 5.9 Hz).

### Example 97

### 1-(4-Trifluoromethoxybenzenesulfonyl)piperidin-4-one O-(2-oxo-2-piperidin-1-ylethyl)oxime

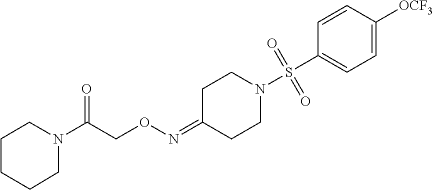

Colorless solid, LC: 100%, retention time: 8.94 min (Method B), M/Z: 464 [M+H]⁺.

¹H-NMR (CDCl₃) δ: 7.82 (2H, d, 8.8 Hz), 7.37 (2H, dd, 0.9, 8.1 Hz), 4.65 (2H, s), 3.51 (2H, t, 5.8 Hz), 3.31 (2H, t, 5.5 Hz), 3.23 (2H, t, 5.8 Hz), 3.19 (2H, t, 6.1 Hz), 2.79 (2H, t, 6.1 Hz), 2.45 (2H, t, 5.7 Hz), 1.62-1.68 (2H, m), 1.52-1.56 (4H, m).

### Example 98

### N-Benzyl-2-[1-(4-trifluoromethoxybenzenesulfonyl)piperidin-4-ylideneaminooxy]acetamide

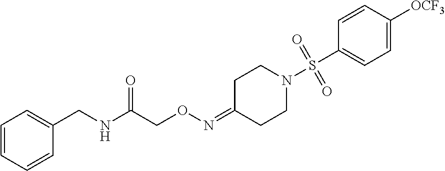

Colorless solid, LC: 100%, retention time: 9.03 min (Method B), M/Z: 486 [M+H]⁺.

¹H-NMR (CDCl₃) δ: 7.81 (2H, d, 8.8 Hz), 7.35-7.38 (2H, m), 7.24-7.32 (5H, m), 6.34 (1H, br, NH), 4.51 (2H, s), 4.49 (2H, d, 5.9 Hz), 3.21 (2H, t, 5.9 Hz), 3.14 (2H, t, 5.9 Hz), 2.70 (2H, t, 5.9 Hz), 2.44 (2H, t, 5.9 Hz), 2.48 (2H, t, 6.2 Hz).

### Example 99

### N-Isobutyl-2-[1-(4-trifluoromethoxybenzenesulfonyl)piperidin-4-ylideneaminooxy]acetamide

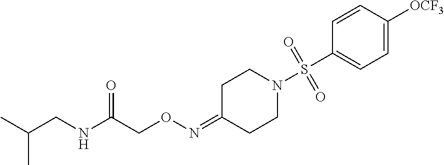

Colorless solid, LC: 100%, retention time: 8.86 min (Method B), M/Z: 452 [M+H]⁺.

¹H-NMR (CDCl₃) δ: 7.82-7.85 (2H, m), 7.36-7.39 (2H, m), 6.08 (1H, br, NH), 4.45 (2H, s), 3.24 (2H, t, 5.9 Hz), 3.19 (2H, t, 6.1 Hz), 3.11 (2H, d, 6.3 Hz), 2.76 (2H, t, 6.1 Hz), 2.48 (2H, t, 6.2 Hz), 1.72-1.79 (1H, m), 0.87 (6H, d, 6.8 Hz).

### Example 100

### N-Cyclohexyl-2-[1-(4-trifluoromethoxybenzenesulfonyl)piperidin-4-ylideneaminooxy]acetamide

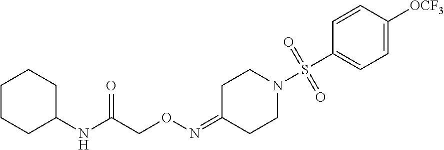

Colorless solid, LC: 100%, retention time: 9.25 min (Method B), M/Z: 478 [M+H]⁺.

¹H-NMR (CDCl₃) δ: 7.83-7.86 (2H, m), 7.36-7.39 (2H, m), 5.89 (1H, br, NH), 4.42 (2H, s), 3.78-3.85 (1H, m), 3.24 (2H, t, 5.9 Hz), 3.19 (2H, t, 6.1 Hz), 2.76 (2H, d, 6.3 Hz), 2.47 (2H, t, 6.1 Hz), 1.85-1.89 (2H, m), 1.56-1.68 (3H, m), 1.32-1.42 (2H, m), 1.09-1.19 (3H, m).

### Example 101

### N-(2-Hydroxyethyl)-2-[1-(4-trifluoromethoxybenzenesulfonyl)piperidin-4-ylideneaminooxy]acetamide

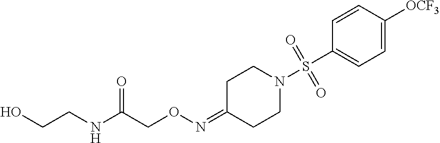

Colorless solid, LC: 100%, retention time: 6.63 min (Method B), M/Z: 440 [M+H]⁺.

¹H-NMR (CDCl₃) δ: 7.82-7.85 (2H, m), 7.36-7.39 (2H, m), 6.53 (1H, br, NH), 4.47 (2H, s), 3.69-3.72 (2H, m), 3.43-3.47 (2H, m), 3.25 (2H, d, 5.9 Hz), 3.21 (2H, t, 6.1 Hz), 2.76 (2H, t, 6.1 Hz), 2.52 (1H, t, 5.0 Hz), 2.47 (2H, t, 6.1 Hz).

### Example 102

### N-Propyl-2-[1-(4-trifluoromethoxybenzenesulfonyl)piperidin-4-ylideneaminooxy]acetamide

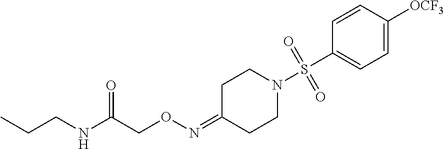

Colorless solid, LC: 100%, retention time: 8.55 min (Method B), M/Z: 438 [M+H]⁺.

¹H-NMR (CDCl₃) δ: 7.82-7.85 (2H, m), 7.36-7.39 (2H, m), 6.08 (1H, br, NH), 4.44 (2H, s), 3.18-3.27 (6H, m), 2.76 (2H, t, 6.1 Hz), 2.48 (2H, t, 5.8 Hz), 1.51 (2H, q, 7.0 Hz), 0.89 (3H, t, 7.4 Hz).

### Example 103

### 3-(2-morpholino-2-oxo-1-(1-(4-(trifluoromethoxy)phenylsulfonyl)piperidin-4-ylideneaminooxy)ethyl)benzonitrile

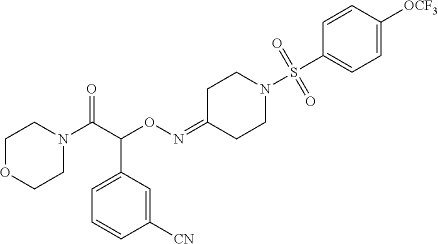

a) Isopropylmagnesium chloride (2M in THF, 6.0 ml, 12 mmol) was added dropwise to a solution of bis(2-dimethylaminoethyl)ether (1.92 g, 12.0 mmol) in THF (50 ml) at 0° C. and stirred for 15 minutes. 3-Iodobenzonitrile (2.29 g, 10.0 mmol) was added to the reaction mixture and stirred at room temperature for 20 minutes. The resulting reaction solution was added dropwise to tert-butyl ethyl oxalate (J. Org. Chem, 1996, 61, 4530) (1.92 g, 11.0 mmol) in THF (50 ml) at at −10° C. and warmed to room temperature for 2 hours. The reaction was quenched with saturated NH₄Cl solution (50 ml), extracted with Et₂O (100 ml×2), washed with brine, dried (MgSO₄) and evaporated under reduced pressure. The residue was purified by column chromatography on silica (EtOAc/Hexanes 10/90-30/70) to give tert-butyl 2-(3-cyanophenyl)-2-oxoacetate (892 mg, 39%).

b) A solution of NaBH₄ (145 mg, 3.82 mmol) in MeOH (10 ml) was added to a solution of tert-butyl 2-(3-cyanophenyl)-2-oxoacetate (885 mg, 3.82 mmol) in MeOH (30 ml) at 0° C. and stirred for 30 minutes. The reaction was quenched with saturated NH₄Cl solution (10 ml) and water (40 ml), extracted with Et₂O (80 ml and 40 ml), washed with brine, dried (MgSO₄) and evaporated under reduced pressure. The residue was purified by column chromatography on silica (EtOAc/Hexanes 15/85-35/65) to give tert-butyl 2-(3-cyanophenyl)-2-hydroxyacetate (217 mg, 24%).

c) Diethyl azodicarboxylate (40% in toluene, 0.500 ml, 1.12 mmol) was added to the mixture of tert-butyl 2-(3-cyanophenyl)-2-hydroxyacetate (217 mg, 0.930 mmol), N-hydroxyphthalimide (167 mg, 1.02 mmol) and triphenylphosphine (274 mg, 1.02 mmol) in THF (10 ml) at 0° C. and stirred at room temperature for 1 hour. The solvent was evaporated under reduced pressure and the residue was purified by column chromatography on silica (EtOAc/Hexanes 35/65) to give tert-butyl 2-(3-cyanophenyl)-2-(1,3-dioxoisoindolin-2-yloxy)acetate (283 mg, 80%).

d) Trifluoroacetic acid (1.5 ml) was added to a solution of tert-butyl 2-(3-cyanophenyl)-2-(1,3-dioxoisoindolin-2-yloxy)acetate (140 mg, 0.370 mmol) in CH₂Cl₂ (1 ml) at 0° C. and stirred at room temperature for 1 hour. The solvent was evaporated under reduced pressure to give 2-(3-cyanophenyl)-2-(1,3-dioxoisoindolin-2-yloxy)acetic acid (119 mg, 100%).

e) N-(3-Dimethylamino-1-propyl)-N′-ethylcarbodiimide hydrochloride (85 mg, 0.44 mmol) was added to a solution of 2-(3-cyanophenyl)-2-(1,3-dioxoisoindolin-2-yloxy)acetic acid (119 mg, 0.370 mmol) and morpholine (0.040 ml 0.44 mmol) in CH₂Cl₂ (6 ml) and stirred at room temperature for 18 hours. The reaction was quenched with water (20 ml), extracted with CHCl₃ (30 ml×2), dried (MgSO₄) and evaporated under reduced pressure. The residue was purified by column chromatography on silica (EtOAc/Hexanes 60/40-80/20) to give 3-(1-(1,3-dioxoisoindolin-2-yloxy)-2-morpholino-2-oxoethyl)benzonitrile (62 mg, 43%).

f) Hydrazine monohydrate (0.014 ml, 0.30 mmol) was added to a solution of 3-(1-(1,3-dioxoisoindolin-2-yloxy)-2-morpholino-2-oxoethyl)benzonitrile (62 mg, 0.16 mmol) in CH₂Cl₂ (2 ml) was stirred at 0° C. for 1.5 hours. The precipitated materials were filtered off and the filtrate was evaporated under reduced pressure. The residue was purified by column chromatography on silica (EtOAc/MeOH 100/0-80/20) to give 3-(1-(aminooxy)-2-morpholino-2-oxoethyl)benzonitrile (26 mg).

g) A solution of 3-(1-(aminooxy)-2-morpholino-2-oxoethyl)benzonitrile (26 mg), 1-(4-(trifluoromethoxy)phenylsulfonyl)piperidin-4-one (23 mg, 0.067 mmol) in EtOH (2 ml) was stirred for 30 minutes and the solvent was evaporated under reduced pressure. The residue was purified by column chromatography on silica (EtOAc/Hexanes 65/35-85/15) to give 3-(2-morpholino-2-oxo-1-(1-(4-(trifluoromethoxy)phenylsulfonyl)piperidin-4-ylideneaminooxy)ethyl)benzonitrile (23 mg, 62%, colorless amorphous, LC: 100%, retention time: 7.02 min (Method A), M/Z: 566).

¹H-NMR (DMSO-d₆) δ: 7.91 (2H, m), 7.81 (2H, m), 7.70 (1H, d, 8.1 Hz), 7.64 (1H, s), 7.63 (1H, d, 8.1 Hz), 7.58 (1H, t, 8.1 Hz), 6.04 (1H, s), 3.36-3.54 (8H, m), 3.09-3.14 (4H, m), 2.68 (2H, m), 2.35 (2H, m).

### Example 104

### 1-(3-(trifluoromethyl)phenylsulfonyl)piperidin-4-one O-4-fluorophenylcarbamoyl oxime

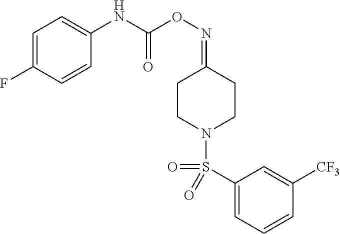

a) Hydroxylamine hydrochloride (0.56 g, 8.14 mmol) was added to a solution of 1-(3-(trifluoromethyl)phenylsulfonyl)piperidin-4-one (2.5 g, 8.14 mmol) in pyridine (10 ml and the whole was stirred at room temperature for 3 hours. The reaction mixture was diluted with ethyl acetate (30 ml) and washed with H₂O and brine, dried over Na₂SO₄ and concentrated in vacuo. The residue was purified by column chromatography (ethyl acetate/petroleum ether: 33/67) to give 1-(3-(trifluoromethyl)phenylsulfonyl)piperidin-4-one oxime (2.4 g, 92%) as a white solid: LCMS: 323 [M+1]⁺.

b) 1-Fluoro-4-isocyanatobenzene (112 mg, 0.74 mmol) and pyridine (98 mg, 1.24 mmol) were added to a solution of 1-(3-(trifluoromethyl)phenylsulfonyl)piperidin-4-one oxime (200 mg, 0.62 mmol) in ethanol (5 ml) and the whole was stirred at room temperature for 17 hours and concentrated in vacuo. The residue was diluted with ethyl acetate (20 ml), washed with brine, dried over Na₂SO₄ and concentrated in vacuo. The residue was purified by column chromatography (ethyl acetate/petroleum ether: 33/67) to give 1-(3-(trifluoromethyl)phenylsulfonyl)piperidin-4-one 0-4-fluorophenylcarbamoyl oxime (100 mg, 45%) as a white solid: LCMS: 482 [M+23]⁺. ¹H NMR (DMSO-d₆) δ: 2.49 (m, 2H), 2.73 (t, 2H), 3.26 (m, 4H), 7.12 (t, 2H), 7.43 (m, 2H), 7.90 (t, 1H), 7.99 (s, 1H), 8.10 (t, 2H), 9.69 (s, 1H).

### Example 105

### 1-(3-(trifluoromethyl)phenylsulfonyl)piperidin-4-one O-ethylcarbamoyl oxime was Prepared as Described in EXAMPLE 104

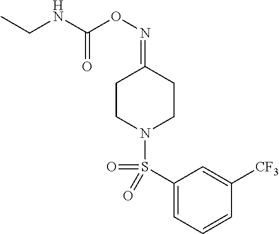

white solid: LCMS: 394 [M+1]⁺. ¹H NMR (DMSO-d₆) δ: 0.83 (t, 3H), 2.46 (m, 2H), 2.65 (t, 2H), 3.03 (t, 2H), 3.21 (m, 4H), 7.30 (t, 1H), 7.88 (t, 1H), 7.98 (s, 1H), 8.09 (t, 2H).

### Example 106

### 1-(3-(trifluoromethyl)phenylsulfonyl)piperidin-4-one O-phenylcarbamoyl oxime was Prepared as Described in EXAMPLE 104

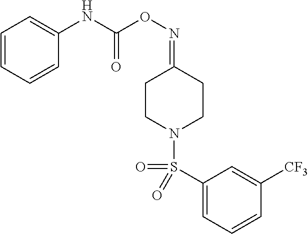

white solid: LCMS: 464 [M+23]⁺. ¹H NMR (DMSO-d₆) δ: 2.49 (m, 2H), 2.74 (t, 2H), 3.23 (m, 4H), 7.00 (t, 1H), 7.27 (t, 2H), 7.43 (d, 2H), 7.90 (t, 1H), 8.00 (s, 1H), 8.10 (t, 2H), 9.67 (s, 1H).

### Example 107

### 1-(3-(trifluoromethyl)phenylsulfonyl)piperidin-4-one O-cyclohexylcarbamoyl oxime was Prepared as Described in EXAMPLE 104

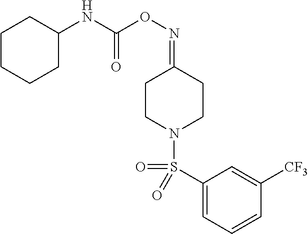

white solid: LCMS: 448 [M+1]⁺. ¹H NMR (DMSO-d₆) δ: 1.17 (m, 6H), 1.51 (d, 1H), 1.67 (m, 4H), 2.47 (m, 2H), 2.65 (t, 2H), 3.22 (m, 4H), 7.07 (d, 1H), 7.88 (t, 1H), 7.98 (s, 1H), 8.09 (t, 2H).

### Example 108

### 1-(3-(trifluoromethyl)phenylsulfonyl)piperidin-4-one O-4-fluorobenzylcarbamoyl oxime was Prepared as Described in EXAMPLE 104

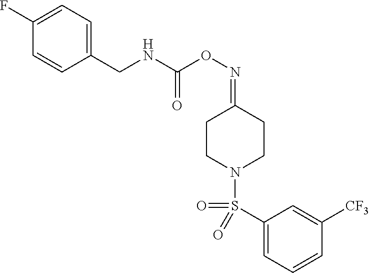

white solid: LCMS: 474 [M+1]⁺. ¹H NMR (DMSO-d₆) δ: 2.49 (m, 2H), 2.66 (t, 2H), 3.20 (m, 4H), 4.18 (d, 2H), 7.09 (t, 2H), 7.25 (m, 2H), 7.88 (q, 2H), 7.98 (s, 1H), 8.09 (t, 2H).

### Example 109

### 1-(3-(trifluoromethyl)phenylsulfonyl)piperidin-4-one O-propylcarbamoyl oxime

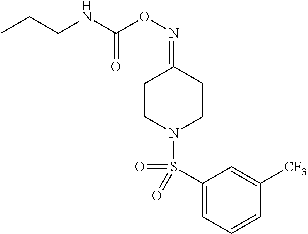

1,1′-Carbonyldiimidazole (201 mg, 1.24 mmol) was added to a solution of 1-(3-(trifluoromethyl)phenylsulfonyl)piperidin-4-one oxime (200 mg, 0.62 mmol) and triethylamine (63 mg, 1.24 mmol) in CH₂Cl₂ (5 ml) and stirred at room temperature for 24 hours. Propan-1-amine (73.2 mg, 1.24 mmol) was added to the reaction mixture and stirred at room temperature for another 7 hours and concentrated in vacuo. The residue was diluted with ethyl acetate (20 ml), washed with brine, dried over Na₂SO₄ and concentrated in vacuo. The residue was purified by column chromatography (ethyl acetate/petroleum ether: 33/67) to give 1-(3-(trifluoromethyl)phenylsulfonyl)piperidin-4-one O-propylcarbamoyl oxime (110 mg, 42%) as a white solid: LCMS: 408 [M+1]⁺. ¹H NMR (DMSO-d₆) δ: 0.78 (t, 3H), 1.38 (q, 2H), 2.46 (m, 2H), 2.65 (t, 2H), 2.94 (q, 2H), 3.20 (m, 4H), 7.29 (t, 1H), 7.88 (t, 1H), 7.98 (s, 1H), 8.09 (t, 2H).

### Example 110

### 1-(3-(trifluoromethyl)phenylsulfonyl)piperidin-4-one O-butylcarbamoyl oxime was Prepared as Described in EXAMPLE 109

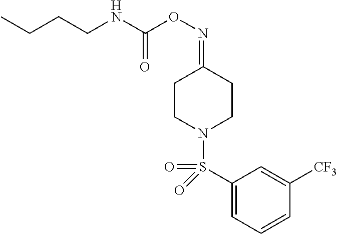

white solid: LCMS: 422 [M+1]⁺. ¹H NMR (DMSO-d₆) δ: 0.82 (t, 3H), 1.21 (q, 2H), 1.34 (q, 2H), 2.46 (m, 2H), 2.65 (t, 2H), 3.00 (q, 2H), 3.21 (m, 4H), 7.27 (t, 1H), 7.88 (t, 1H), 7.98 (s, 1H), 8.09 (t, 2H).

### Example 111

### 1-(3-(trifluoromethyl)phenylsulfonyl)piperidin-4-one O-(4-fluorophenyl)(methyl)carbamoyl oxime

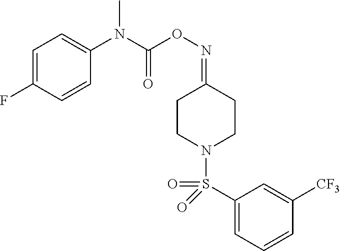

A solution of 1-(3-(trifluoromethyl)phenylsulfonyl)piperidin-4-one oxime (200 mg, 0.62 mmol) in tetrahydrofuran (10 ml) was added over 1 hour to a suspension of trichloromethyl chloroformate (120 mg, 0.62 mmol) activated charcoal (20 mg) in tetrahydrofuran (30 ml) at 0° C. After stirring at room temperature for 15 hours, the reaction mixture was filtered over silicagel and the filtrate was concentrated in vacuo. The residue was dissolved in tetrahydrofuran (15 ml), 4-Fluoro-N-methylaniline (93.0 mg, 0.74 mmol) and N,N-diisopropylethylamine (240 mg, 1.86 mmol) were added and the whole was stirred at room temperature for 16 hours. The reaction was quenched with H₂O (10 ml) and aqueous 1 N HCl solution (pH=3). The aqueous phase was extracted with CH₂Cl₂ (70 ml×3) and the combined organic phase was washed with aqueous NaHCO₃ solution and brine, dried over Na₂SO₄, filtered and concentrated in vacuo. The residue was purified by column chromatography (ethyl acetate/petroleum ether: 33/67) to give 1-(3-(trifluoromethyl)phenylsulfonyl)piperidin-4-one O-(4-fluorophenyl)(methyl)carbamoyl oxime (200 mg, 71%) as a white solid: LCMS: 474 [M+1]⁺. ¹H NMR (DMSO-d₆) δ: 2.47 (m, 4H), 3.10 (t, 2H), 3.21 (m, 5H), 7.18 (m, 2H), 7.32 (m, 2H),7.88 (t, 1H), 7.97 (s, 1H), 8.10 (m, 2H).

### Example 112

### 1-(3-(trifluoromethyl)phenylsulfonyl)piperidin-4-one O-isoxazol-3-ylcarbamoyl oxime was prepared as described in EXAMPLE 111

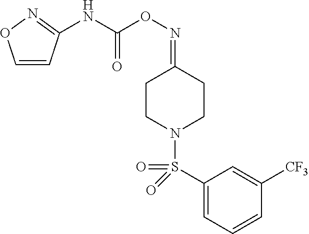

white solid: LCMS: 433 [M+1]⁺. ¹H NMR (DMSO-d₆) δ: 2.48 (m, 2H), 2.73 (t, 2H), 3.25 (m, 4H), 6.71 (d, 1H), 7.88 (t, 1H), 8.00 (s, 1H), 8.09 (m, 2H), 8.75 (d, 1H), 10.7 (s, 1H).

### Example 113

### 1-(3-(trifluoromethyl)phenylsulfonyl)piperidin-4-one O-isoquinolin-3-ylcarbamoyl oxime was Prepared as Described in EXAMPLE 111

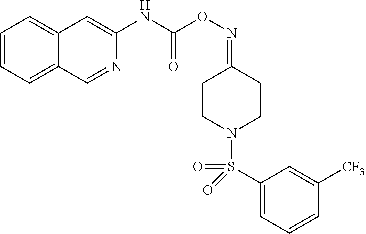

white solid: LCMS: 493 [M+1]⁺. ¹H NMR (DMSO-d₆) δ: 2.48 (m, 2H), 2.79 (t, 2H), 3.27 (m, 4H), 7.49 (m, 1H), 7.68 (m, 1H), 7.89 (t, 2H), 8.02 (m, 2H), 8.10 (m, 3H), 9.10 (s, 1H) , 10.1 (s, 1H).

### Example 114

### 1-(3-(trifluoromethyl)phenylsulfonyl)piperidin-4-one O-4-chlorophenylcarbamoyl oxime was Prepared as Described in EXAMPLE 109

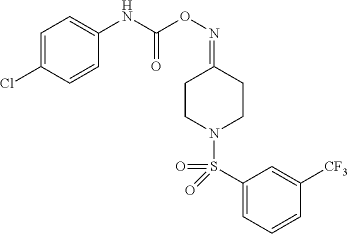

white solid: LCMS: 476 [M+1]⁺. ¹H NMR (DMSO-d₆) δ: 2.50 (m, 2H), 2.74 (m, 2H), 3.25 (m, 4H), 7.33 (m, 2H), 7.47 (m, 21-1), 7.89 (t, 1H), 8.00 (s, 1H), 8.10 (m, 2H), 9.83 (s, 1H).

### Example 115

### 1-(3-(trifluoromethyl)phenylsulfonyl)piperidin-4-one O-4-methoxyphenylcarbamoyl oxime was Prepared as Described in EXAMPLE 109

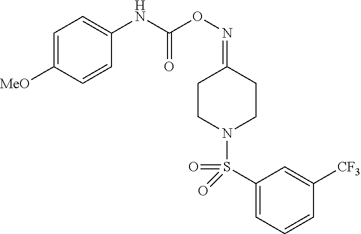

white solid: LCMS: 472 [M+1]⁺. ¹H NMR (DMSO-d₆) δ: 2.48 (m, 2H), 2.73 (t, 2H), 3.26 (m, 4H), 3.68 (s, 3H), 6.85 (m, 2H), 7.32 (d, 2H), 7.89 (t, 1H), 8.00(s, 1H), 8.10(m, 2H), 9.45 (s, 1H).

### Example 116

### 1-(3-(trifluoromethyl)phenylsulfonyl)piperidin-4-one O-3-fluorophenylcarbamoyl oxime was Prepared as Described in EXAMPLE 111

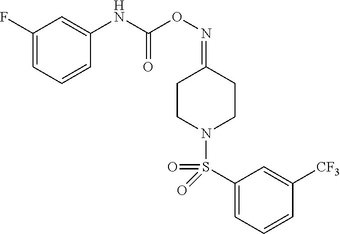

white solid: LCMS: 482 [M+23]⁺. ¹H NMR (DMSO-d₆) δ: 2.48 (m, 2H), 2.74 (t, 2H), 3.25 (m, 4H), 6.83 (m, 1H), 7.32 (m, 3H), 7.89 (t, 1H), 8.08 (d, 1H), 8.10 (t, 2H), 9.91 (s, 1H).

### Example 117

### 1-(3-(trifluoromethyl)phenylsulfonyl)piperidin-4-one O-pyridin-2-ylcarbamoyl oxime was Prepared as Described in EXAMPLE 111

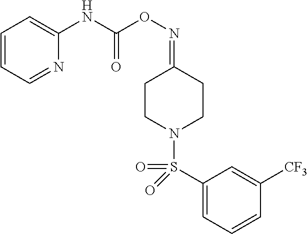

white solid: LCMS: 443 [M+1]⁺. ¹H NMR (DMSO-d₆) δ: 2.48 (m, 2H), 2.77 (t, 2H), 3.25 (m, 4H), 7.05 (m, 1H), 7.77 (m, 2H), 7.89 (t, 1H), 8.00 (s, 1H), 8.10 (d, 2H), 8.26 (m, 1H), 10.08 (s, 1H).

### Example 118

### 1-(3-(trifluoromethyl)phenylsulfonyl)piperidin-4-one O-quinolin-3-ylcarbamoyl oxime was Prepared as Described in EXAMPLE 111

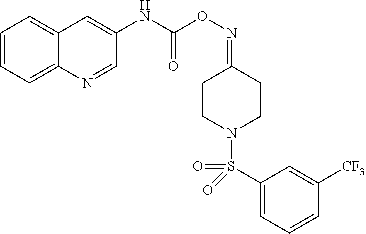

white solid: LCMS: 493 [M+1]⁺. ¹H NMR (DMSO-d₆) δ: 2.54 (t, 2H), 2.78 (t, 2H), 3.26 (m, 4H), 7.59 (m, 2H), 7.90 (m, 3H), 8.01 (s, 1H), 8.11 (d, 2H), 8.42 (d, 1H), 8.88 (d, 1H), 10.16 (s, 1H).

### Example 119

### 1-(3-(trifluoromethyl)phenylsulfonyl)piperidin-4-one O-2-(4-fluorophenoxy)ethylcarbamoyl oxime was Prepared as Described in EXAMPLE 111

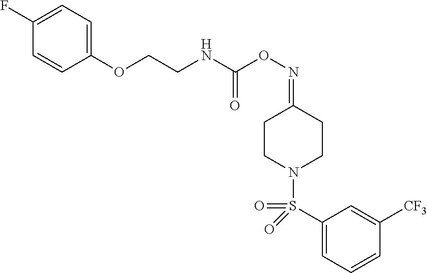

white solid: LCMS: 503 [M+1]⁺. ¹H NMR (DMSO-d₆) δ: 2.50 (m, 2H), 2.68 (m, 2H), 3.25 (m, 4H), 3.38 (m, 2H), 3.97 (t, 2H),6.93 (m, 2H), 7.10 (m, 2H), 7.53 (t, 1H), 7.92 (m, 1H), 8.00 (s, 1H), 8.11 (m, 2H).

### Example 120

### 4-((1-(3-(trifluoromethyl)phenylsulfonyl)piperidin-4-ylideneaminooxy)carbonylamino)benzonitrile was Prepared as Described in EXAMPLE 111

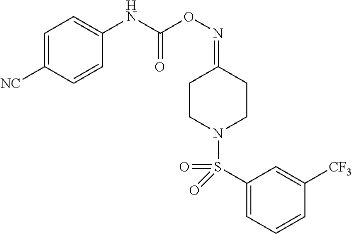

white solid: LCMS: 426 [M+1]⁺. ¹H NMR (DMSO-d₆) δ: 2.52 (m, 2H), 2.75 (m, 2H), 3.26 (m, 4H), 7.61 (m, 2H), 7.74 (m, 2H), 7.89 (m, 1H), 8.08 (m, 1H), 8.10 (m, 2H), 10.2 (s, 1H).

### Example 121

### 1-(3-(trifluoromethyl)phenylsulfonyl)piperidin-4-one O-pyridin-3-ylcarbamoyl oxime was Prepared as Described in EXAMPLE 111

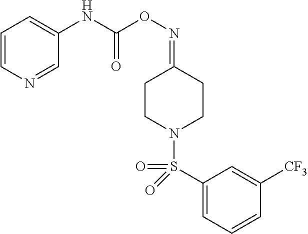

white solid: LCMS: 443 [M+1]⁺. ¹H NMR (DMSO-d₆) δ: 2.52 (m, 2H), 2.75 (m, 2H), 3.24 (m, 4H), 7.32 (m, 1H), 7.92 (m, 3H), 8.22 (m, 3H), 8.61 (d, 1H), 9.90 (s, 1H).

### Example 122

### 1-(3-(trifluoromethyl)phenylsulfonyl)piperidin-4-one O-quinolin-2-ylcarbamoyl oxime was Prepared as Described in EXAMPLE 111

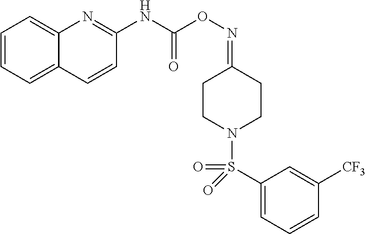

white solid: LCMS: 493 [M+1]⁺. ¹H NMR (DMSO-d₆) δ: 2.50 (m, 2H), 2.81 (m, 2H), 3.24 (m, 4H), 7.46 (m, 1H), 7.76 (m, 2H), 7.90 (m, 2H), 8.10 (m, 4H), 8.32 (m, 1H), 10.5 (s, 1H).

### Example 123

### 1-(3-(trifluoromethyl)phenylsulfonyl)piperidin-4-one O-2-fluorophenylcarbamoyl oxime was Prepared as Described in EXAMPLE 111

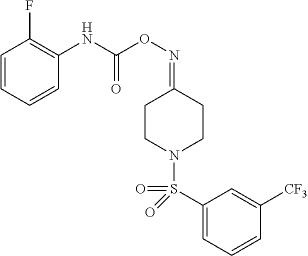

white solid: LCMS: 460 [M+1]⁺. ¹H NMR (DMSO-d₆) δ: 2.53 (m, 2H), 2.795 (m, 2H), 3.28 (m, 4H), 7.20 (m, 3H), 7.65 (m, 1H), 7.96 (m, 2H), 8.14 (m, 2H), 9.41 (s, 1H).

### Example 124

### 1-(3-(trifluoromethyl)phenylsulfonyl)piperidin-4-one O-benzo[d][1,3]dioxol-5-ylcarbamoyl oxime was Prepared as Described in EXAMPLE 109

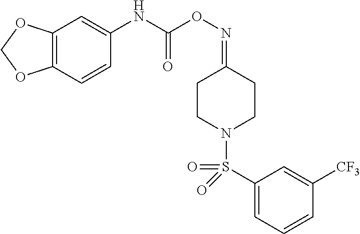

white solid: LCMS: 486 [M+1]⁺. ¹H NMR (DMSO-d₆) δ: 2.51 (m, 2H), 2.75 (t, 2H), 3.28 (m, 4H), 5.97 (s, 2H), 6.85 (m, 2H), 7.09 (d, 1H), 7.91 (t, 1H), 8.02 (s, 1H), 8.12 (t, 2H), 9.56 (s, 1H).

### Example 125

### 1-(3-(trifluoromethyl)phenylsulfonyl)piperidin-4-one O-pyridin-4-ylcarbamoyl oxime was Prepared as Described in EXAMPLE 109

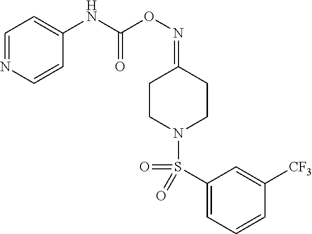

white solid: LCMS: 443 [M+1]⁺. ¹H NMR (DMSO-d₆) δ: 2.51 (m, 2H), 2.78 (t, 2H), 3.27 (m, 4H), 7.44 (m, 2H), 7.92 (t, 1H), 8.02 (s, 1H), 8.13 (t, 2H), 8.39 (m, 2H), 10.2 (s, 1H).

### Example 126

### 1-(3-(trifluoromethyl)phenylsulfonyl)piperidin-4-one O-thiazol-2-ylcarbamoyl oxime was Prepared as Described in EXAMPLE 109

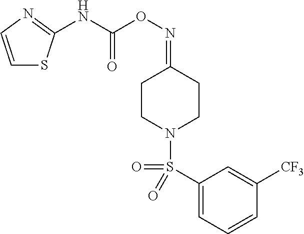

white solid: LCMS: 449 [M+1]⁺. ¹H NMR (DMSO-d₆) δ: 2.49 (m, 2H), 2.74 (t, 2H), 3.24 (m, 4H), 7.18 (d, 1H), 7.38 (d, 1H), 7.89 (t, 1H), 8.00 (s, 1H), 8.11 (m, 2H), 11.8 (s, 1H).

### Example 127

### 1-(3-(trifluoromethyl)phenylsulfonyl)piperidin-4-one O-cyclopropylcarbamoyl oxime was Prepared as Described in EXAMPLE 109

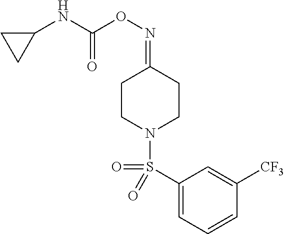

white solid: LCMS: 406 [M+1]⁺. ¹H NMR (DMSO-d₆) δ: 0.43 (m, 2H), 0.59 (m, 2H), 2.50 (m, 3H), 2.67 (m, 2H), 3.22 (m, 4H), 7.48 (s, 1H), 7.91 (t, 1H), 8.00(s, 1H), 8.12(m, 2H).

### Example 128

### (4-fluorophenylamino)(1-(3-(trifluoromethyl)benzoyl)piperidin-4-ylideneaminooxy)methanone

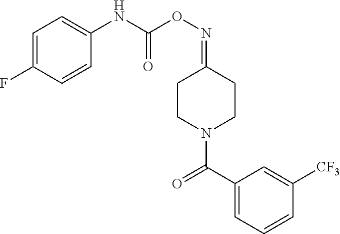

a) A solution of 4-fluorophenylisocyanate (1.38 g, 10.0 mmol) in tetrahydrofuran (40 ml) was added to a solution of ethyl acetohydroxamate (1.03 g, 10.0 mmol) and triethylamine (1.40 ml, 10.0 mmol) in tetrahydrofuran (60 ml) at 0° C. and the whole was stirred at 0° C. for 1 hour and concentrated in vacuo. The residue was diluted with 1,4-dioxane (11.25 ml) and aqueous 70% perchloric acid solution (5.25 ml) was added dropwise at 0° C. over 5 minutes. After stirring at room temperature for 10 minutes, the reaction was quenched with aqueous NaOH solution, extracted with ethyl acetate (100 ml×2), washed with brine, dried over MgSO₄ and concentrated in vacuo. The residue was purified by column chromatography (ethyl acetate/n-hexane: 40/60 to 65/35) and thus obtained solid was triturated with n-hexane to give N-(aminooxycarbonyl)-4-fluoroaniline (880 mg, 69%) as a pale-yellow solid.

b) 1-(3-Dimethylaminopropyl)-3-ethylcarboiimide hydrochloride (1.15 g, 6.00 mmol) was added to a solution of 4-piperidone monohydrate hydrochloride (768 mg, 5.00 mmol), 1-hydroxybenzotriazole monohydrate (743 mg, 5.50 mmol), 3-(trifluoromethyl)benzoic acid (951 mg, 5.00 mmol) and triethylamine (0.840 ml, 6.00 mmol) in N,N-dimethylformamide (25 ml). The reaction mixture was stirred at room temperature for 2 hours and at 60° C. for 2.5 hours and quenched with saturated aqueous NaHCO₃ (50 ml). The aqueous phase was extracted with ethyl acetate (50 ml×3) and the combined organic phase was washed with brine, dried over MgSO₄, filtered and concentrated in vacuo. The residue was purified by column chromatography (ethyl acetate/n-hexane: 50/50 to 75/25) to give 1-(3-(trifluoromethyl)benzoyl)piperidin-4-one (1.30 g, 96%) as pale-yellow oil.

c) N-(Aminooxycarbonyl)-4-fluoroaniline (51 mg, 0.30 mmol) was added to a solution of 1-(3-(trifluoromethyl)benzoyl)piperidin-4-one (81 mg, 0.30 mmol) in ethanol (6 ml) and stirred at room temperature for 1 hour. The reaction was quenched with H₂O (20 ml) and extracted by chloroform (20 ml×2). The combined organic phase was dried over MgSO₄, filtered and concentrated in vacuo. The residue was purified by column chromatography (ethyl acetate/n-hexane: 50/50 to 70/30) and thus obtained solid was recrystallized from ethyl acetate/n-hexane to give (4-fluorophenylamino)(1-(3-(trifluoromethyl)benzoyl)piperidin-4-ylideneaminooxy)methanone (95 mg, 75%) as a white solid: LCMS: 424 [M+1]⁺. ¹H NMR (DMSO-d₆) δ: 2.55-2.81 (m, 4H), 3.49 (m, 2H), 3.80 (m, 2H), 7.17 (m, 2H), 7.51 (m, 2H),7.72 (m, 1H), 7.79 (m, 1H), 7.85 (m, 2H), 9.76 (s, 1H).

### Example 129

### 4-(4-fluorophenylcarbamoyloxvimino)-N-(3-(trifluoromethyl)phenyl)piperidine-1-carboxamide

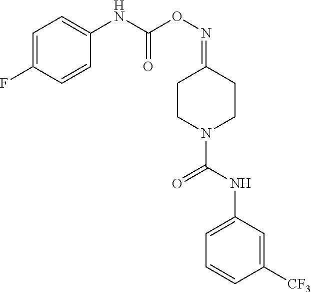

a) A solution of 3-(trifluoromethyl)phenyl isocyanate (1.16 g, 6.00 mmol) in N,N-dimethylformamide (5 ml) was added to a solution of 4-piperidone monohydrate hydrochloride (768 mg, 5.00 mmol) and N,N-diisopropylethylamine (1.89 ml, 11.0 mmol) in N,N-dimethylformamide (25 ml) at 0° C. and stirred at room temperature for 12 hours. The reaction was quenched with H₂O (50 ml), extracted by ethyl acetate (100 ml×3), washed with brine, dried over MgSO₄, filtered and concentrated in vacuo. The residual solid was triturated with ethyl acetate/n-hexane to give 4-oxo-N-(3-(trifluoromethyl)phenyl)piperidine-1-carboxamide (1.14 g, 79%) as a pale-yellow solid.

b) 4-(4-fluorophenylcarbamoyloxyimino)-N-(3-(trifluoromethyl)phenyl)piperidine-1-carboxamide was prepared as described in the procedure c in EXAMPLE 128.

pale-pink solid: LCMS: 439 [M+1]⁺. ¹H NMR (DMSO-d₆) δ: 2.52 (m, 2H), 2.73 (m, 2H), 3.68 (m, 4H), 7.16 (m, 2H), 7.28 (m, 1H), 7.51 (m, 3H), 7.77 (m, 1H), 7.95 (s, 1H), 8.97 (s, 1H), 9.75 (s, 1H).

### Example 130

### 1-(4-(4-fluorophenylcarbamoyloxyimino)piperidin-1-yl)-2-(3-(trifluoromethyl)phenyl)ethanone was Prepared as Described in EXAMPLE 128

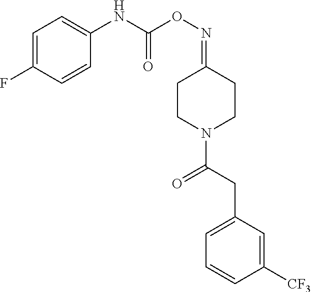

white solid: LCMS: 438 [M+1]⁺. ¹H NMR (DMSO-d₆) δ: 2.47 (m, 2H), 2.67 (m, 2H), 3.63-3.77 (m, 4H), 3.93 (s, 211), 7.16 (m, 2H), 7.49-7.62 (m, 6H), 9.75 (d, 1H).

### Example 131

### 3-(trifluoromethyl)phenyl 4-(4-fluorophenylcarbamoyloxyimino)piperidine-1-carboxylate

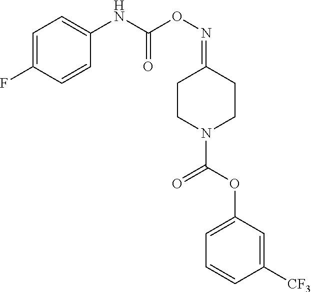

a) A solution of 3-(trifluoromethyl)phenyl chloroformate (1.42 g, 6.00 mmol) in CH₂Cl₂ (10 ml) was added to a solution of 4-piperidone monohydrate hydrochloride (768 mg, 5.00 mmol) and N,N-diisopropylethylamine (1.89 ml, 11.0 mmol) in CH₂Cl₂ (40 ml) at 0° C. over 30 minutes and stirred at room temperature for 6.5 hours. The reaction was quenched with H₂O (50 ml), extracted by CHCl₃ (50 ml). The combined organic phase was dried over MgSO₄, filtered and concentrated in vacuo. The residue was purified by column chromatography (ethyl acetate/n-hexane: 15/85 to 50/50) to give 3-(trifluoromethyl)phenyl 4-oxopiperidine-1-carboxylate (583 mg, 40%) as colorless oil.

b) 3-(trifluoromethyl)phenyl 4-(4-fluorophenylcarbamoyloxyimino)piperidine-1-carboxylate was prepared as described in the procedure c in EXAMPLE 128.

white solid: LCMS: 440 [M+1]⁺. ¹H NMR (DMSO-d₆) δ: 2.61 (m, 2H), 2.82 (m, 2H), 3.65 (m, 2H), 3.81 (m, 2H), 7.17 (m, 2H), 7.52 (m, 3H), 7.66 (m, 3H), 9.78 (s, 1H).

### Example 132

### 2-(4-(4-fluorophenylcarbamoyloxyimino)piperidin-1-yl)-2-oxo-N-(3-(trifluoromethyl)phenyl)acetamide

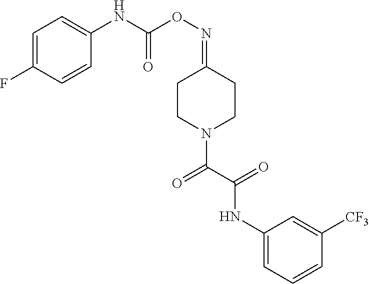

a) A solution of ethyl chloroglyoxylate (2.04 g, 1.50 mmol) in toluene (5 ml) was added dropwise to a mixture of 3-trifluoromethylaniline (1.61 g, 10.0 mmol) in aqueous 2 N NaOH solution (10 ml), H₂O (20 ml) and toluene (25 ml) at 0° C. and stirred at 0° C. for 6 hours. n-Hexane (60 ml) was added to the reaction mixture and the resulting precipitation was collected, washed with H₂O and n-hexane to give ethyl 2-oxo-2-(3-(trifluoromethyl)phenylamino)acetate (2.28 g, 87%) as a white solid.

b) Aqueous 2 N NaOH solution (13 ml, 26 mmol) was added to a solution of ethyl 2-oxo-2-(3-(trifluoromethyl)phenylamino)acetate (2.28 g, 8.74 mmol) in ethanol (23 ml) at 45° C. and stirred at 45° C. for 45 minutes. The reaction was quenched with aqueous 2 N HCl solution (14 ml, pH=3) and concentrated in vacuo. The residue was diluted with H₂O (50 ml) and the resulting precipitaion was collected and washed with H₂O and n-hexane to give 2-oxo-2-(3-(trifluoromethyl)phenylamino)acetic acid (1.78 g, 87%) as a white solid.

c) 2-Oxo-2-(4-oxopiperidin-1-yl)-N-(3-(trifluoromethyl)phenyl)acetamide was prepared from 2-oxo-2-(3-(trifluoromethyl)phenylamino)acetic acid as described in the procedure b in EXAMPLE 128.

d) 2-(4-(4-fluorophenylcarbamoyloxyimino)piperidin-1-yl)-2-oxo-N-(3-(trifluoromethyl)phenyl)acetamide was prepared as described in the procedure c in EXAMPLE 128.

pale-yellow amorphous: LCMS: 467 [M+1]⁺. ¹H NMR (DMSO-d₆) δ: 2.59 (m, 2H), 2.79 (m, 2H), 3.75 (m, 4H), 7.16 (m, 2H), 7.51 (m, 3H), 7.62 (m, 1H), 7.88 (m, 1H), 8.16 (s, 1H), 9.79 (s, 1H).

### Example 133

### 4-(4-fluorophenylcarbamoyloxyimino)-N-(3-(trifluoromethyl)phenyl)piperidine-1-sulfonamide

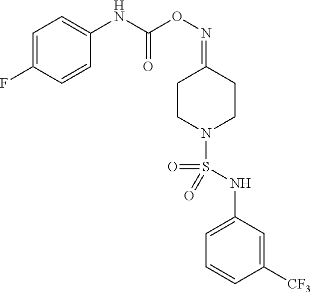

a) A solution of 1,4-dioxa-8-azaspiro[4.5]decane (1.46 g, 10.0 mmol) and 4-dimethylaminopyridine (1.22 g, 10.0 mmol) in CH₂Cl₂ (10 ml) was added to a solution of SO₂Cl₂ (0.890 ml, 11.0 mmol) in CH₂Cl₂ (10 ml) at −78° C. over 1 hour and the whole was stirred at room temperature for 3 hours and concentrated in vacuo. The residue was purified by column chromatography (ethyl acetate/n-hexane: 15/85 to 50/50) to give 1,4-dioxa-8-azaspiro[4.5]decane-8-sulfonyl chloride (1.80 g, 75%) as a colorless solid.

b) A solution of 1,4-dioxa-8-azaspiro[4.5]decane-8-sulfonyl chloride (967 mg, 4.00 mmol) in CH₂Cl₂ (10 ml) was added to a solution of 3-trifluoromethylaniline (645 mg, 4.00 mmol) and N,N-diisopropylethylamine (1.65 ml, 9.60 mmol) in CH₂Cl₂ (10 ml) and pyridine (12 ml) at 0° C. over 1 hour and stirred at 65° C. for 12 hours. The reaction mixture was diluted with ethyl acetate (500 ml), washed with aqueous 2 N HCl solution (200 ml) and brine, dried over MgSO₄, filtered and concentrated in vacuo. The residue was purified by column chromatography (ethyl acetate/n-hexane: 35/65 to 55:45) to give N-(3-(trifluoromethyl)phenyl)-1,4-dioxa-8-azaspiro[4.5]decane-8-sulfonamide (626 mg, 41%) as pale-yellow oil.

c) The mixture of N-(3-(trifluoromethyl)phenyl)-1,4-dioxa-8-azaspiro[4.5]decane-8-sulfonamide (620 mg, 1.63 mmol) and aqueous 2 N HCl solution (5 ml) in acetone (2.5 ml) was refluxed for 12 hours. The reaction mixture was poured into aqueous 2 N NaOH solution (6 ml) and H₂O (15 ml) and extracted with ethyl acetate (30 ml×3), washed with brine, dried over MgSO₄, filtered and concentrated in vacuo. The residue was purified by column chromatography (ethyl acetate/n-hexane: 40/60 to 60:40) to give 4-oxo-N-(3-(trifluoromethyl)phenyl)piperidine-1-sulfonamide (595 mg, 90%) as colorless oil.

d) 4-(4-fluorophenylcarbamoyloxyimino)-N-(3-(trifluoromethyl)phenyl)piperidine-1-sulfonamide was prepared as described in the procedure c in EXAMPLE 128.

pale-yellow solid: LCMS: 475 [M+1]⁺. ¹H NMR (DMSO-d₆) δ: 2.43 (m, 2H), 2.65 (m, 2H), 3.40 (m, 4H), 7.15 (m, 2H), 7.42 (m, 1H), 7.49 (m, 4H), 7.57 (m, 1H), 9.73 (s, 1H), 10.49 (s, 1H).

### Example 134

### 1-(m-tolylsulfonyl)piperidin-4-one O-4-fluorophenylcarbamoyl oxime

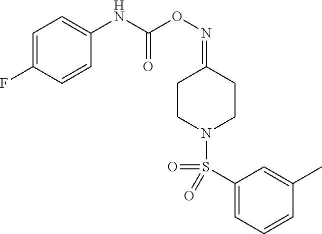

a) Hydroxylamine hydrochloride (2.50 g, 36.0 mmol) was added to a solution of 1-Boc-4-piperidone (6.10 g, 30.0 mmol) and sodium acetate (2.95 g, 36.0 mmol) in ethanol (60 ml) and the whole was stirred at room temperature for 1 hour. The reaction mixture was concentrated in vacuo and the residue was diluted with ethyl acetate (120 ml) and washed with saturated aqueous NaHCO₃ (30 ml) and brine, dried over MgSO₄ and concentrated in vacuo. The residual solid was recrystallized from ethyl acetate/n-hexane to give tert-butyl 4-(hydroxyimino)piperidine-1-carboxylate (6.18 g, 96%) as a white solid.

b) A solution of 4-fluorophenylisocyanate (775 mg, 5.65 mmol) in tetrahydrofuran (20 ml) was added to a solution of tert-butyl 4-(hydroxyimino)piperidine-1-carboxylate (1.00 g, 4.67 mmol) and triethylamine (0.783 ml, 5.65 mmol) in tetrahydrofuran (30 ml) at 0° C. over 10 minutes and stirred at 0° C. for 4 hours. The mixture was concentrated in vacuo and the residual solid was recrystallized from ethyl acetate/n-hexane to give tert-butyl 4-(4-fluorophenylcarbamoyloxyimino)piperidine-l-carboxylate (1.57 g, 96%) as a white solid.

c) 4 N HCl solution in 1,4-dioxane (4 ml) was added to a solution of tert-butyl 4-(4-fluorophenylcarbamoyloxyimino)piperidine-1-carboxylate (703 mg, 2.00 mmol) in 1,4-dioxane (4 ml) at 0° C. and stirred at room temperature for 4 hours. The reaction was quenched with saturated aqueous NaHCO₃ (5 ml), extracted with CHCl₃/methanol (10:1, 50 ml×5), dried over MgSO₄ and concentrated in vacuo. The residue was purified by column chromatography (CHCl₃/methanol: 95/5 to 80:20) to give piperidin-4-one O-4-fluorophenylcarbamoyl oxime (428 mg, 69%) as yellow syrup.

d) A solution of m-toluenesulfonyl chloride (70 mg, 0.36 mmol) in CH₂Cl₂ (2 ml) was added to a solution of piperidin-4-one O-4-fluorophenylcarbamoyl oxime (93 mg, 0.30 mmol) and N,N-diisopropylethylamine (0.062 ml, 0.36 mmol) in CH₂Cl₂ (4 ml) at 0° C. and stirred at 0° C. for 1 hour. The reaction was quenched with saturated aqueous NaHCO₃ (15 ml), extracted with CHCl₃ (20 ml×2), dried over MgSO₄ and concentrated in vacuo. The residue was purified by column chromatography (ethyl acetate/n-hexane: 35/65 to 55:45) to give 1-(m-tolylsulfonyl)piperidin-4-one O-4-fluorophenylcarbamoyl oxime (103 mg, 83%) as colorless foam: LCMS: 406 [M+1]⁺. ¹H NMR (DMSO-d₆) δ: 2.41 (s, 3H), 2.52 (m, 2H), 2.74 (m, 2H), 3.18 (m, 4H), 7.14 (m, 2H), 7.45-7.60 (m, 6H), 9.71 (s, 1H).

### Example 135

### 3-(4-(4-fluorophenylcarbamoyloxyimino)piperidin-1-ylsulfonyl)benzonitrile was Prepared as Described in EXAMPLE 134

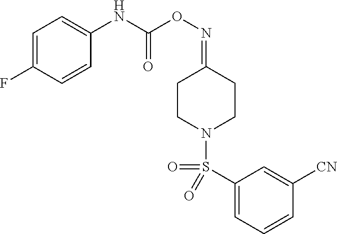

white solid: LCMS: 417 [M+1]⁺. ¹H NMR (DMSO-d₆) δ: 2.52 (m, 2H), 2.76 (m, 2H), 3.27 (m, 4H), 7.14 (m, 2H), 7.48 (m, 2H), 7.86 (m, 1H), 8.11 (m, 1H), 8.20 (m, 1H), 8.27 (s, 1H), 9.73 (s, 1H).

Compounds of the invention have been tested in the calcium mobilization and/or electrophysiological assay for N-type calcium channel blocking activity, which are described in detail above. Some compounds described have also been tested in the calcium mobilization assay for L-type calcium channel blocking activity, which is described in detail above. Representative values are presented in TABLE 2.

TABLE 2
Evaluation of the tested compounds as N-type calcium channel
(NTCC) blockers and L-type calcium channel (LTCC) blockers
after a calcium mobilization in vitro assay

| Example | | NTCC | LTCC |
|---|---|---|---|
| Number | Compound Name | IC₅₀ (nM) | IC₅₀ (nM) |
| 2 | N-Phenyl-2-[1-(4- | 420 | >20000 |
| | trifluoromethoxybenzenesulfonyl)piperidin-4- | | |
| | ylideneaminooxy]acetamide | | |
| 3 | N-Methyl-N-phenyl-2-[1-(4- | 1200 | 8500 |
| | trifluoromethoxybenzenesulfonyl)piperidin-4- | | |
| | ylideneaminooxy]acetamide | | |
| 98 | N-Benzyl-2-[1-(4- | 1100 | >20000 |
| | trifluoromethoxybenzenesulfonyl)piperidin-4- | | |
| | ylideneaminooxy]acetamide | | |
| 99 | N-Isobutyl-2-[1-(4- | 300 | >20000 |
| | trifluoromethoxybenzenesulfonyl)piperidin-4- | | |
| | ylideneaminooxy]acetamide | | |
| 100 | N-Cyclohexyl-2-[1-(4- | 410 | >20000 |
| | trifluoromethoxybenzenesulfonyl)piperidin-4- | | |
| | ylideneaminooxy]acetamide | | |
| 102 | N-Propyl-2-[1-(4- | 210 | >20000 |
| | trifluoromethoxybenzenesulfonyl)piperidin-4- | | |
| | ylideneaminooxy]acetamide | | |
| 104 | 1-(3-(trifluoromethyl)phenylsulfonyl)piperidin-4- | 240 | >20000 |
| | one O-4-fluorophenylcarbamoyl oxime | | |
| 131 | 3-(trifluoromethyl)phenyl 4-(4- | 1800 | Not |
| | fluorophenylcarbamoyloxyimino)piperidine-1- | | evaluated |
| | carboxylate | | |

Having now fully described this invention, it will be understood by those of ordinary skill in the art that the same can be performed within a wide and equivalent range of conditions, formulations and other parameters without affecting the scope of the invention or any embodiment thereof.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All patents and publications cited herein are fully incorporated by reference herein in their entirety.

1. A compound having Formula I: 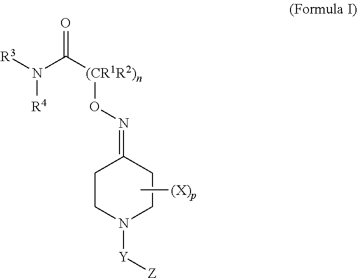
or a pharmaceutically acceptable salt, a prodrug or a solvate thereof, wherein:
Y is CO or SOₘ;
Z is hydrogen, optionally substituted lower alkyl, optionally substituted lower alkenyl, optionally substituted lower alkynyl, optionally substituted cycloalkyl, optionally substituted bicycloalkyl, optionally substituted cycloalkenyl, optionally substituted aryl, optionally substituted heterocyclyl, NR⁵R⁶, OR⁵, SR⁵, COR⁵ or CONR⁵R⁶,
R¹ and R² are each independently hydrogen, halogen, cyano, optionally substituted lower alkyl, optionally substituted lower alkenyl, optionally substituted lower alkynyl, optionally substituted cycloalkyl, optionally substituted cycloalkenyl, optionally substituted aryl or optionally substituted heterocyclyl, or
R¹ and R² taken together, with the carbon atom to which they are attached, form optionally substituted cycloalkane, optionally substituted cycloalkene, optionally substituted bicycloalkane, or optionally substituted heterocycle;
R³ and R⁴ are each independently hydrogen, cyano, optionally substituted lower alkyl, optionally substituted lower alkenyl, optionally substituted lower alkynyl, optionally substituted cycloalkyl, optionally substituted bicycloalkyl, optionally substituted cycloalkenyl, optionally substituted aryl, optionally substituted heterocyclyl, NR⁵R⁶ or OR⁵; or
R³ and R⁴ taken together, with the nitrogen atom to which they are attached, form optionally substituted heterocycle;
R⁵ and R⁶ are each independently hydrogen, optionally substituted lower alkyl, optionally substituted lower alkenyl, optionally substituted lower alkynyl, optionally substituted cycloalkyl, optionally substituted bicycloalkyl, optionally substituted cycloalkenyl, optionally substituted aryl or optionally substituted heterocyclyl;
each X is independently ═O, optionally substituted lower alkyl, optionally substituted lower alkenyl, optionally substituted lower alkynyl, halogen, cyano, nitro, NR⁵R⁶, OR⁵, SR⁵, COR⁵, COOR⁵, CONR⁵R⁶, NR⁵COR⁵, OCOR⁵, SOR⁵, SO₂R⁵, SO₃R⁵, SONR⁵R⁶, SO₂NR⁵R⁶,
NRSOR⁵, or NRSO₂R⁵,
n is 0, 1, 2, 3, 4 or 5,
m is 1 or 2 and
p is 0, 1 or 2,
provided that
when n is 1, R³ is NH₂ and R¹, R² and R⁴ are each H, then Y—Z is not COOEt,
when Y is CO, Z is not optionally substituted (3,4-dihydro-1H-benzo[b]azepin-5(2H)ylidene)methyl, or
when n is 0, Y is SOₘ.

2. The compound of claim 1, wherein n is 1.

3. The compound of claim 1 or 2, wherein Y is SO₂.

4. The compound of claim 1, wherein Z is optionally substituted aryl or optionally substituted heterocyclyl.

5. The compound of claim 1, wherein R³ is cyano, optionally substituted lower alkyl, optionally substituted cycloalkyl, optionally substituted bicycloalkyl, optionally substituted cycloalkenyl, optionally substituted aryl, optionally substituted heterocyclyl, NR⁵R⁶ or OR⁵, R⁴ is hydrogen, or R³and R⁴ taken together, with the nitrogen atom to which they are attached, form heterocyclyl, and R⁵ and R⁶ are each independently hydrogen or optionally substituted lower alkyl

6. A pharmaceutical composition, comprising the compound of claim 1 and a pharmaceutically acceptable carrier.

7. A method of treating, preventing or ameliorating a disorder responsive to the blockade of calcium channels in a mammal suffering from said disorder, comprising administering to a mammal in need of such treatment, prevention or amelioration an effective amount of a compound of claim 1.

8. The method of claim 7, wherein a disorder responsive to the blockade of N-type calcium channels is treated, prevented or ameliorated.

9. A method for treating, preventing or ameliorating stroke, neuronal damage resulting from head trauma, epilepsy, pain, migraine, a mood disorder, schizophrenia, a neurodegenerative disorder, depression, anxiety, a psychosis, hypertension or cardiac arrhythmia in a mammal, comprising administering an effective amount of a compound of any claim 1.

10. The method of claim 9, wherein the method is for treating, preventing or ameliorating pain selected from chronic pain, acute pain, and surgical pain.

11. A method of modulating calcium channels in a mammal, comprising administering to the mammal at least one compound of claim 1.

12. The method of claim 11, wherein the N-type calcium channel is modulated.

13. A compound having Formula I of claim 1, wherein the compound is ³H, ¹¹C, or ¹⁴C radiolabeled.

14. A method of screening a candidate compound for the ability to bind to a receptor using a radiolabeled compound of claim 13, comprising a) introducing a fixed concentration of the radiolabeled compound to the receptor to form a mixture; b) titrating the mixture with a candidate compound; and c) determining the binding of the candidate compound to said receptor.

15. Use of a compound of Formula I as claimed in claim 1 in the manufacture of a medicament for treating, preventing or ameliorating stroke, neuronal damage resulting from head trauma, epilepsy, pain, migraine, a mood disorder, schizophrenia, a neurodegenerative disorder, depression, anxiety, a psychosis, hypertension or cardiac arrhythmia in a mammal.

16. Use of a compound of Formula I as claimed in claim 1 in the manufacture of a medicament for treating, preventing or ameliorating pain selected from chronic pain, acute pain, and surgical pain.

17. A pharmaceutical composition for modulating calcium channels in a mammal, comprising the compound having Formula I′: 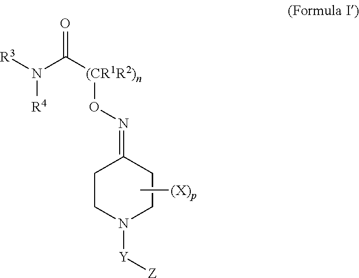
or a pharmaceutically acceptable salt, a prodrug and a solvate thereof, wherein:
Y is CO or SOₘ;
Z is hydrogen, optionally substituted lower alkyl, optionally substituted lower alkenyl, optionally substituted lower alkynyl, optionally substituted cycloalkyl, optionally substituted bicycloalkyl, optionally substituted cycloalkenyl, optionally substituted aryl, optionally substituted heterocyclyl, NR⁵R⁶, OR⁵, SR⁵, COR⁵ or CONR⁵R⁶,
R¹ and R² are each independently hydrogen, halogen, cyano, optionally substituted lower alkyl, optionally substituted lower alkenyl, optionally substituted lower alkynyl, optionally substituted cycloalkyl, optionally substituted cycloalkenyl, optionally substituted aryl or optionally substituted heterocyclyl, or
R¹ and R² taken together, with the carbon atom to which they are attached, form optionally substituted cycloalkane, optionally substituted cycloalkene, optionally substituted bicycloalkane, or optionally substituted heterocycle;
R³ and R⁴ are each independently hydrogen, cyano, optionally substituted lower alkyl, optionally substituted lower alkenyl, optionally substituted lower alkynyl, optionally substituted cycloalkyl, optionally substituted bicycloalkyl, optionally substituted cycloalkenyl, optionally substituted aryl, optionally substituted heterocyclyl, NR⁵R⁶ or OR⁵; or
R³ and R⁴ taken together, with the nitrogen atom to which they are attached, form optionally substituted heterocycle;
R⁵ and R⁶ are each independently hydrogen, optionally substituted lower alkyl, optionally substituted lower alkenyl, optionally substituted lower alkynyl, optionally substituted cycloalkyl, optionally substituted bicycloalkyl, optionally substituted cycloalkenyl, optionally substituted aryl or optionally substituted heterocyclyl,
each X is independently ═O, optionally substituted lower alkyl, optionally substituted lower alkenyl, optionally substituted lower alkynyl, halogen, cyano, nitro, NR⁵R⁶, OR⁵, SR⁵, COR⁵, COOR⁵, CONR⁵R⁶, NR⁵COR⁵, OCOR⁵, SOR⁵, SO₂R⁵, SO₃R⁵, SONR⁵R⁶, SO₂NR⁵R⁶, NRSOR⁵, or NRSO₂R⁵,
n is 0, 1, 2, 3, 4 or 5,
m is 1 or 2 and
p is 0, 1 or 2,
and a pharmaceutically acceptable carrier.
